(19) United States
(12) Patent Application Publication
(10) Pub. No.: US 2009/0203722 A1
(43) Pub. Date: Aug. 13, 2009

(54) Novel compositions and methods for enhancing potency or reducing adverse side effects of opiold agonists

(75) Inventors: Barry M. Sherman, Hillsborough, CA (US); Mary M. Remien, San Francisco, CA (US); Remi Barbier, San Francisco, CA (US); Kathleen Dumas, Cupertino, CA (US); Grant L. Schoenhard, San Carlos, CA (US)

Correspondence Address: BELL, BOYD & LLOYD, LLP, P.O. Box 1135, CHICAGO, IL, 60690, US

(73) Assignee: PAIN THERAPEUTICS, INC., South San Francisco, CA (US)

(21) Appl. No.: 11/456,079

(22) Filed: Jul. 6, 2006

Related U.S. Application Data
(63) Continuation of application No. 10/228,835, filed on Aug. 26, 2002, now abandoned.
Application No. 10/228,835 is a continuation of application No. 10/047,367, filed on Jan. 14, 2002, now abandoned.
Application No. 10/047,367 is a continuation of application No. 09/849,721, filed on May 4, 2001, now abandoned.
Application No. 09/849,721 is a continuation of application No. 09/756,331, filed on Jan. 8, 2001, now abandoned.
Application No. 09/756,331 is a continuation of application No. 09/566,071, filed on May 5, 2000, now abandoned.
Application No. 09/566,071 is a continuation of application No. PCT/US00/12493, filed on May 5, 2000.

(51) Int. Cl. A61K 31/485; A61K 31/135; A61P 25/00

(52) U.S. Cl. 514/282; 514/646

(57) ABSTRACT

The invention generally relates to novel compositions and methods with an opioid agonist and an opioid antagonist to differentially dose a human subject so as to either enhance analgesic potency without attenuating an adverse side effect of the agonist, or alternatively maintain the analgesic potency of the agonist while attenuating an adverse side effect of the agonist. The invention additionally relates to novel opioid compositions and methods for the gender-based dosing of men and women.

### CROSS-REFERENCE TO RELATED APPLICATIONS

This application claims the priority of the following U.S. Patent Application Nos. 60/202,227 filed May 5, 2000 (provisional); 60/202,268 filed May 5, 2000 (provisional); 09/756,331 filed Jan. 8, 2001, which is a continuation of Ser. No. 09/566,071 filed May 5, 2000; 60/244,482 filed Oct. 30, 2000 (provisional); 60/245,110 filed Nov. 1, 2000 (provisional); and 60/246,235 filed Nov. 2, 2000 (provisional); and PCT/US00/12493 [WO 00/67739] filed May 5, 2000. The applications cited above are hereby incorporated herein by reference in their entirety to provide continuity of disclosure.

### FIELD OF THE INVENTION

The present invention relates to novel compositions and methods, including gender-based compositions and methods, for enhancing potency or reducing adverse side effects of opioid agonists in humans. The present invention also relates to novel compositions and methods with an opioid agonist and an opioid antagonist to differentially dose a human subject, including men and/or women, so as to either enhance analgesic potency without attenuating an adverse side effect of the agonist, or alternatively maintain the analgesic potency of the agonist while attenuating an adverse side effect of the agonist.

### BACKGROUND OF THE INVENTION

Opioid agonists, including morphine sulfate (hereafter called morphine or MS), have been marketed for many years and are widely used for the relief of moderate to severe acute and chronic pain. The potency of oral morphine is less than that of parenteral morphine, however, the use of the oral product for chronic pain control has increased dramatically in the past decade. An opioid agonist, such as morphine, exerts its primary effects on the central nervous system and organs containing smooth muscle, and acts as an agonist interacting with stereospecific and saturable binding sites or receptors in the brain, spinal cord, and other tissues. The principal therapeutic actions are analgesia and sedation.

Opioid antagonists are generally accepted for use in the treatment of human conditions or ailments for reversing opioid toxicity and overdoses, and in preventing abuse of opioid agonists, such as heroin or morphine. For these uses, the antagonist such as naloxone or naltrexone is used in relatively high concentrations in order to effectively block the activity and/or effects of the opioid agonist by antagonizing the opioid agonist at opioid receptors on nociceptive neurons.

Naloxone (4,5-epoxy-3,14-dihydroxy-17-(2-prophenyl)morphinan-6-one) was the first of these compounds to be synthesized in 1960 and is considered a “pure” antagonist, i.e., exhibiting virtually no agonist activity. Naloxone became the preferred regime for the treatment of acute opioid toxicity. Since naloxone exhibits a relatively short duration in the body, it became clear that a longer acting agent having similarly pure antagonist character would be even more advantageous. Naltrexone (17-(cyclopropylmethyl)-4,5-epoxy-3,14-dihydroxy-morphinan-6-one) was developed in 1965 and has greater potency and longer action than its N-allyl cogener, naloxone, and is active when given orally. For example, 50 mg dosage forms of naltrexone, are marketed as ReVia® in the United States or Trexan in other countries. Nalmefene (6-methylene-6-desoxy-N-cyclopropyl-methyl-14-hydroxydihydroxydihydronor-morphine) was also developed as a long acting, orally available, potent opioid antagonist, and has also been characterized as a pure antagonist. These drugs are presently commercially available in certain dosage forms, and are so far as is known, the only opioid antagonists characterized as pure antagonists which have received governmental approval for administration to humans.

Opioid agonists, such as morphine, are commonly used by clinicians in the treatment of moderate to severe acute and chronic pain. The analgesic activity of these agents contributes to their pharmacological effects on a large number of inhibitory opioid receptors on sensory nerve cells that receive and transmit pain signals in the nervous system; the role of these receptors is to inhibit the transmission of pain signals into the brain. The precise mechanisms of opioid agonists such as morphine are not known, although morphine, for example, is believed to act preferentially at mu-opiate receptors on neurons in the central and peripheral nervous system. In addition to pain relief, other actions of opioid agonists such as morphine, in human subjects, include adverse side effects such as inhibition of gastrointestinal motility (e.g., leading to constipation), respiratory depression (especially at high-doses), peripheral vasodilation (e.g., leading to orthostatic hypotension), dizziness, sedation/drowsiness, nausea, vomiting, headache, pruritus, dry mouth, difficulty in urination, dependence, mood swings, and clouded sensorium.

Opioid antagonists have been widely used in high-doses for the treatment of overdoses of opioid agonists and to prevent abuse of opioid agonists such as heroin or morphine (e.g., 50 mg naltrexone). For these uses, doses must be relatively high in order to be therapeutically effective (i.e., block) the analgesic potency and the side effects of the opioid agonist, by antagonizing the agonist at opioid receptors on nociceptive neurons.

Crain and Shen (Brain Research 757: 176-190 (1997)) reported that opioid agonists not only activate inhibitory opioid receptors leading to analgesia but also simultaneously activate a smaller group of excitatory opioid receptors on sensory nerve cells. These effects on the excitatory oploid receptors were proposed to weaken opioid induced analgesia and under certain conditions actually enhance pain. Surprisingly, Crain and Shen (e.g., U.S. Pat. No. 5,512,578 reissued as RE 36,457) showed that co-administration of remarkably low-doses of an opioid antagonist, such as naloxone or naltrexone on the order of ng/kg, when administered to mice with morphine or similar opioid agonists selectively blocked their effects on excitatory, but not inhibitory, opioid receptors, thus markedly enhancing the analgesic potency of opioid agonists. These surprising results of Crain and Shen have been described in U.S. Pat. Nos. 5,472,943; 5,512,578 reissued as RE 36,457; 5,580,876 and 5,767,125, which are directed to methods for selectively enhancing the analgesic potency of a bimodally-acting opioid agonist and simultaneously attenuating anti-analgesia, hyperalgesia, hyperexcitability, physical dependence and/or tolerance effects associated with the administration of the bimodally-acting opioid agonist. These methods comprise administering to a subject an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist. Also included in these patents are methods for treating pain in a subject comprising administering to the subject an analgesic or sub-analgesic amount of a bimodally-acting opioid agonist and an amount of an excitatory opioid receptor antagonist effective to enhance the analgesic potency of the bimodally-acting opioid agonist and simultaneously attenuate anti-analgesia, hyperalgesia, hyperexcitability, physical dependence and/or tolerance effects of the bimodally-acting opioid agonist. Also included are methods for treating an opiate addict comprising administering to the opiate addict an amount of an excitatory opioid receptor antagonist either alone or in combination with a bimodally-acting opioid agonist effective to attenuate physical dependence caused by a bimodally-acting opioid agonist and enhance the analgesic potency of a bimodally-acting opioid agonist. Also included are compositions comprising an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist in a subject administered the composition. In all of these studies, the antagonist simultaneously enhanced potency while attenuating such adverse effects. Two clinical studies on postsurgical hysterectomy patients [Joshi, et al., Anesthesiol. 90: 1007-1011 (1999); Gan et al., Anesthesiol. 87: 1075-1081 (1997)] demonstrated that cotreatment of women with PCA/IV morphine together with a low-dose of the opioid antagonist naloxone (IV) or nalmefene (IV) enhanced potency of morphine in varying cumulative doses of morphine over a 24 hour period. Adverse side effects were attenuated in these studies. Nothing in these studies with women suggested or related to any gender-based effect on either opioid-induced analgesia and/or the adverse effects associated with opioids.

In a recent review of gender differences in pharmacokinetics and pharmacodynamics [Beierle et al., Intl. J. Clin. Pharmacol. Ther. 37 (11): 529-547 (1999)], it was pointed out that until 1993, women were excluded from clinical phase I and early phase II trials. Therefore, for most drugs, including analgesics, there is a real paucity of information on sex differences in the pharmacokinetics as well as in the dose-response relationship or adverse effects of these drugs. The U.S. Food and Drug Administration (FDA) recognized this situation and developed new guidelines for drug research in 1993. Sex-related analgesic responses, including a summary and critique of animal and human studies and discrepancies between such studies were recently reviewed by Levine and his colleagues [Miaskowski et al., Chapter 11, pages 209-230, Editor: Fillingim, IASP Press, Seattle, Sex Gender and Pain (2000)]. In another recent review, Miaskowski and Levine [Pain Forum 8(1): 34-44 (1999)], summarize data from human studies on sex-related differences in responses to opioid analgesics, particularly kappa opioids.

Certain gender-based pain responses have been reported in both animal and human clinical studies [for reviews, see Fillingham and Maixner, Pain Forum 4: 209-221 (1995); Unruh, Pain 65: 123-167 (1996); Miaskowski et al. (2000), supra.] Gender-based differences in analgesia and anti-analgesia have recently been shown by Levine and his colleagues in patients with postoperative pain with several kappa opioid agonists, e.g., butorphanol [Gear et al., Nature, 2: 1248-1250 (1996)]; pentazocine [Gear et al., Neuroscience Let., 205: 207-209 (1996)]; nalbuphine [Gear et al., Pain 83: 339-345 (1999)]; and nalbuphine in combination with naloxone, an opioid antagonist [Gear et al., J. Pain 1: 122-127 (2000)], but not with the mu opioid agonist morphine [Gordon et al., Neuroscience 69(2): 345-349 (1995)]. According to Levine and his colleagues, kappa opioid receptor agonists are unique in their gender-related effects. Studies in rats and mice evaluating the role of mu opioid agonists and antagonists show gender-based effects, although the results of these studies are contradictory and appear to be dependent upon both species and gender (for reviews, see Kest et al., J. Pharmacol. Exper. Therapeutics, 289: 1370-1375 (1999); and Kest et al., Anesthesiology, 93: 539-547 (2000)).

### SUMMARY OF THE INVENTION

The present invention relates to novel compositions and methods for enhancing potency or reducing adverse side effects of opioid agonists in humans. The present invention is directed to compositions and methods for the differential dosing of human subjects with opioid agonists and low doses of opioid antagonists to yield either (1) enhancement of analgesic potency of the agonist without attenuation (e.g., reduction) or increase of one or more of the adverse side effects associated with that dose of agonist in humans, or (2) maintenance of analgesic potency of the agonist with attenuation (e.g., reduction) of one or more of the adverse side effects associated with that dose of agonist in humans. The present invention is based on surprising results from human clinical trials that demonstrate that the analgesic potency of opioid agonists can be dissociated from the opioid-related adverse side effects in humans. One novel composition and dosing method of the invention utilizes a dose of agonist with a low dose of antagonist that gives more pain relief in men and/or women but with essentially the same adverse side effect(s) of agonist alone. A second novel composition and dosing method of the invention utilizes a dose of agonist with a low dose of antagonist that gives essentially the same pain relief in men and/or women as agonist alone, but with attenuated (e.g., reduced) adverse side effect(s). The maintained potency with attenuated side effect(s) is accomplished without increasing or decreasing the cumulative daily dose of agonist. Thus, at appropriate differential dosing of humans according to the invention, a low dose of antagonist surprisingly can enhance analgesia with no increase in side effects or suppress side effects with no loss in analgesia.

The present invention is also directed to novel compositions and methods for gender-based dosing of non-kappa opioid receptor agonists, preferably mu opioid receptor agonists such as morphine sulfate, and/or opioid antagonists such as naltrexone. Such compositions and methods are designed to achieve appropriate and even optimal analgesia, and are useful for treating moderate or severe pain, wherein the pain is either acute or chronic. Appropriate and even optimal analgesia is only possible when pain relief is enhanced, without enhancing and preferably attenuating, adverse side effects of such agonists or antagonists.

The present invention is based in part on additional surprising results from human clinical trials that demonstrate that the analgesic potency and/or the adverse side effects of morphine sulfate, a mu opioid receptor agonist, is gender-specific. Additionally surprising are gender-specific responses to such agonists, including the discovery of the problem that current methods of treatment with such agonists result in hypo-analgesia in men, including anti-analgesia, while similar treatment of women results in analgesia but with significant adverse side effects. Compositions and methods described herein provide for the first time a solution to problems related to previously undiscovered differences in drug effects, including pain intensity differences, pain relief or adverse side effects, using such agonists in women and men, including those effects associated with the management of pain.

The present invention is also directed to novel compositions and methods for gender-based dosing of opioid antagonists, such as naltrexone, to avoid hypo-analgesia. This is based in part on surprising results from human clinical trials that the responses to naltrexone, an opioid antagonist, are also gender-specific. Additionally surprising are results that indicate that such an antagonist can act as a partial opioid agonist on opioid receptors differentially in women and men.

The present invention is also directed to novel compositions and methods for gender-based dosing of combinations of non-kappa opioid receptor agonists, preferably mu opioid receptor agonists, with opioid antagonists to achieve optimal analgesia. This is based in part on surprising results from human clinical trials that there are gender-based differences in the interactions between such agonists and antagonists.

The present invention provides compositions and methods for administering to a woman, for example, a dose of a non-kappa opioid receptor agonist, preferably a mu opioid receptor agonist, that alone is analgesic in women but hypo-analgesic in men, while attenuating one or more adverse side effects of such agonists in women. The present invention also provides compositions and methods for administering to a man, for example, a dose of a non-kappa opioid receptor agonist, preferably a mu opioid receptor agonist, that alone is hypo-analgesic in men but analgesic in women, without substantially enhancing one or more adverse side effects of such agonists in men.

The present invention is also directed to novel compositions and methods for ethnic-based dosing of combinations of opioid receptor agonists, including non-kappa opioid receptor agonists, and preferably mu opioid receptor agonists, with opioid antagonists to achieve optimal analgesia. This is based in part on surprising results from human clinical trials that there are ethnic-based differences in the interactions between such agonists and antagonists.

The present invention provides compositions and methods for administering to a Hispanic man, for example, a dose of opioid receptor agonist, preferably a non-kappa opioid receptor agonist, most preferably a mu opioid receptor agonist, that alone is analgesic in Hispanic men but hypo-analgesic in non-Hispanic men, while attenuating one or more adverse side effects of such agonists in Hispanic men. The present invention also provides compositions and methods for administering to a Black man, for example, a dose of a opioid receptor agonist, preferably a non-kappa opioid receptor agonist, most preferably a mu opioid receptor agonist, that alone is hypo-analgesic in Black men but analgesic in women and/or Hispanic men, without substantially enhancing one or more adverse side effects of such agonists in Black men.

The present invention thus provides compositions and methods for the differential dosing in women and men, for example, with non-kappa opioid receptor agonists, preferably mu opioid receptor agonists, based on co-treatment of such agonists with low doses of opioid receptor antagonists. Specifically provided are compositions and methods of enhancing pain relief or attenuating pain intensity in men comprising administering, for example, to a man a hypo-analgesic dose (including a non-analgesic or anti-analgesic dose) of a mu opioid receptor agonist and a dose of an opioid antagonist that in combination enhances pain relief or attenuates pain intensity. Such compositions and methods convert non-responder human subjects, (e.g., men) into responders. Also specifically provided are compositions and methods of enhancing pain relief or attenuating pain intensity, for example, in women comprising administering to a woman an analgesic dose of a mu opioid receptor agonist and a dose of opioid antagonist that in combination enhances pain relief or attenuates pain intensity comparable to that of the analgesic dose of agonist alone but with attenuation of one or more adverse side effects of the agonist. Thus, compositions and methods for providing, enhancing or maintaining pain relief, as well as for attenuating pain intensity, are specifically provided as gender-specific compositions and methods for women or men.

The present invention provides compositions and methods for the differential dosing in women and men of non-kappa opioid receptor agonists, preferably mu opioid receptor agonists, based on gender-based differences in their pharmacodynamic effects, including pain relief or adverse side effects, from gender-specific interactions of such agonists in women and men. Compositions and methods are provided for administering a non-kappa opioid receptor agonist, preferably a mu opioid receptor agonist, at a gender-specific compensatory dose based on different pharmacodynamic effects in women and men, wherein such a gender-specific compensatory dose provides enhancement of analgesia and/or attenuation of an adverse side effect of the agonist.

The present invention provides compositions and methods that include a non-kappa opioid receptor agonist, preferably a mu opioid receptor agonist, and an opioid antagonist in amounts that are useful for men only, or for women only, or for both men and women, based on the differences described herein.

### BRIEF DESCRIPTION OF THE FIGURES

FIG. 1 shows the total pain relief (TOTPAR) results at 4 hours (see also Table 4) in the five study groups in Example 1: placebo; morphine; morphine and low dose (0.01 mg) naltrexone (NTX); morphine and mid dose (0.1 mg) NTX; and morphine and high dose (1.0 mg) NTX.

FIG. 2 shows the sum of pain intensity differences (SPID) results at 4 hours (see also Table 5) in the five study groups in Example 1: placebo; morphine; morphine and low dose (0.01 mg) naltrexone (NTX); morphine and mid dose (0.1 mg) NTX; and morphine and high dose (1.0 mg) NTX.

FIG. 3 shows the time to onset of meaningful pain relief results (see also Table 6) in the five study groups in Example 1: placebo; morphine; morphine and low dose (0.01 mg) naltrexone (NTX); morphine and mid dose (0.1 mg) NTX; and morphine and high dose (1.0 mg) NTX.

FIGS. 4 and 5 show the time to remedication (rescue medication) up to 8 and 24 hours, respectively (see also Table 7) in the five study groups in Example 1: placebo; morphine; morphine and low dose (0.01 mg) naltrexone (NTX); morphine and mid dose (0.1 mg) NTX; and morphine and high dose (1.0 mg) NTX.

FIG. 6 shows the pain relief results (see also Table 9) for 4 hours in the five study groups in Example 1: placebo represented as small diamonds (⋄); morphine represented as squares (□); morphine and low dose (0.01 mg) NTX represented as large circles (O); morphine and mid dose (0.1 mg) NTX represented as triangles (Δ); and morphine and high dose (1.0 mg) NTX represented as larger diamonds (⋄).

FIG. 7 shows the pain intensity difference (PID) results (see also Table 10) for 4 hours in the five study groups in Example 1: placebo; morphine; morphine and low dose (0.01 mg) naltrexone (NTX); morphine and mid dose (0.1 mg) NTX; and morphine and high dose (1.0 mg) NTX.

FIG. 8 shows a summary of adverse side effects of nausea, vomiting, dizziness, headache, somnolence (sedation) or pruritus in the five study groups in Example 1: placebo; morphine; morphine and low dose (0.01 mg) naltrexone (NTX); morphine and mid dose (0.1 mg) NTX; and morphine and high dose (1.0 mg) NTX.

FIGS. 9B and 9C show the summary of pain intensity difference (SPID) results at 4 hours (SPID-4) (see also Tables 18A and 18B) for women and men, respectively, in the five study groups as described in Example 2: placebo; morphine (60 mg); morphine and low-dose (0.01 mg) naltrexone (NTX); morphine and mid-dose (0.1 mg) NTX; morphine and high-dose (1.0 mg) NTX.

FIGS. 10A and 10B show the time to onset of meaningful pain relief results (see also Tables 19A and 19B) in the five study groups as described in Example 2: placebo; morphine; morphine and low-dose (0.01 mg) naltrexone (NTX); morphine and mid-dose (0.1 mg) NTX; and morphine and high-dose (1.0 mg) NTX, for women and men, respectively.

FIGS. 11A and 12A for women, and 11B and 12B for men, show the time to remedication (rescue medication) up to 8 and 24 hours, respectively (see also Tables 20A and 20B) in the five study groups as described in Example 2: placebo; morphine; morphine and low-dose (0.01 mg) naltrexone (NTX); morphine and mid-dose (0.1 mg) NTX; and morphine and high-dose (1.0 mg) NTX, for women and men, respectively.

FIGS. 13A for women, and 13B for men, show the pain relief results (see also Tables 22A and 22B) in the five study groups as described in Example 2: placebo; morphine; morphine and low-dose (0.01 mg) naltrexone (NTX); morphine and mid-dose (0.1 mg) NTX; and morphine and high-dose (1.0 mg) NTX, for women and men, respectively.

FIGS. 14A for women and 14B for men show the pain intensity difference (PID) results (see also Tables 23A and 23B) in the five study groups as described in Example 2: placebo; morphine; morphine and low-dose (0.01 mg) naltrexone (NTX); morphine and mid-dose (0.1 mg) NTX; and morphine and high-dose (1.0 mg) NTX, for women and men, respectively.

FIGS. 15A for women (see also Tables 26A and 26B) and 15B for men (see also Tables 26C and 26D) show a summary of adverse side effects of nausea, vomiting, dizziness, headache, somnolence (sedation) or pruritus in the five study groups as described in Example 2: placebo; morphine (60 mg); morphine and low-dose (0.01 mg) naltrexone (NTX); morphine and mid-dose (0.1 mg) NTX; morphine and high-dose (1.0 mg) NTX.

FIG. 16 shows the time to onset of meaningful pain relief results (see also Table 32A) for subjects in the six study groups as described in Example 3: placebo; morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIG. 17 shows the time to onset of analgesia results (see also Table 32B) for subjects in the six study groups as described in Example 3: placebo; morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIG. 18 shows the time to remedication (rescue medication) up to 8 hours (see also Table 33) for subjects in the six study groups as described in Example 3: placebo; morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIG. 19 shows the time to remedication (rescue medication) up to 8 and 24 hours, (see also Table 33) for subjects in the six study groups as described in Example 3: placebo; morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIG. 20 shows the pain relief (PR) results (see also Table 35) for subjects in the six study groups as described in Example 3: placebo; morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIG. 21 shows the pain intensity differences (PID) results (see also Table 36) for subjects in the six study groups as described in Example 3: placebo, morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIG. 22 shows the summary of adverse side effects (see also Tables 39A and 39B) of nausea, vomiting, dizziness, headache, somnolence (sedation) or pruritus for subjects in the six study groups as described in Example 3: placebo; morphine (60 mg); naltrexone (0.01 mg); morphine and low-dose (0.001 mg) naltrexone (NTX); morphine and mid-dose (0.01 mg) naltrexone; morphine and high-dose (0.1 mg) NTX.

FIGS. 23A, 23B and 23C show the summary of pain intensity difference (SPID) results at 4 hours (SPID-4) (see also Tables 44A and 44B) for the total study population, followed by women and men, respectively, in the six study groups as described in Example 4: placebo; morphine (60 mg); naltrexone (0.01 mg); morphine and low-dose (0.001 mg) naltrexone (NTX); morphine and mid-dose (0.01 mg) NTX; morphine and high-dose (0.1 mg) NTX.

FIGS. 24A and 24B show the time to onset of meaningful pain relief results (see also Tables 45A and 45B) in the six study groups as described in Example 4: placebo; morphine (60 mg); naltrexone (0.01 mg); morphine and low-dose (0.001 mg) naltrexone (NTX); morphine and mid-dose (0.01 mg) NTX; morphine and high-dose (0.1 mg) NTX for men and women respectively.

FIGS. 25A and 26A for women, and 25B and 26B for men, show the time to remedication (rescue medication) up to 8 and 24 hours, respectively (see also Tables 46A and 46B) in the six study groups as described in Example 4: placebo; morphine; naltrexone (0.01 mg); morphine and low-dose (0.001 mg) naltrexone (NTX); morphine and mid-dose (0.01 mg) NTX; and morphine and high-dose (0.1 mg) NTX, for women and men, respectively.

FIGS. 27A for women, and 27B for men, show the pain relief results (see also Tables 48A and 48B) in the six study groups as described in Example 4: placebo; morphine; naltrexone (0.01 mg); morphine and low-dose (0.001 mg) naltrexone (NTX); morphine and mid-dose (0.01 mg) NTX; and morphine and high-dose (0.1 mg) NTX, for women and men, respectively.

FIGS. 28A for women and 28B for men show the pain intensity difference (PID) results (see also Tables 49A and 49B) in the six study groups as described in Example 4: placebo; morphine (60 mg); naltrexone (0.01 mg); morphine and low-dose (0.001 mg) naltrexone (NTX); morphine and mid-dose (0.01 mg) NTX; and morphine and high-dose (0.1 mg) NTX, for women and men, respectively.

FIGS. 29A for women (see also Tables 52A and 52B) and 29B for men (see also Tables 52C and 52D) show a summary of adverse side effects of nausea, vomiting, dizziness, headache, somnolence (sedation) or pruritus in the six study groups described in Example 4: placebo; morphine (60 mg); naltrexone (0.01 mg); morphine and low-dose (0.001 mg) naltrexone (NTX); morphine and mid-dose (0.01 mg) NTX; morphine and high-dose (0.1 mg) NTX.

FIG. 30 shows the total pain relief (TOTPAR) results (see also Table 56) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 31 shows the summary of pain intensity difference (SPID) results at 4 hours (SPID-4), at 6 hours (SPID-6), and at 8 hours (SPID-8) (see also Table 57) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 32 shows the time to onset of meaningful pain relief results (see also Table 58A) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 33 shows the time to onset to analgesia results (see also Table 58B) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 34 shows the time to remedication (rescue medication) up to 8 hours (see also Table 59) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 35 shows the pain relief (PR) results (see also Table 61) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 36 shows the pain intensity differences (PID) results (see also Table 62) for subjects in the six study groups as described in Example 5: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F).

FIG. 37 shows the summary of adverse side effects (see also Table 65) of nausea, vomiting, dizziness, headache, somnolence (sedation) or pruritus for subjects in the six study groups as described in Example 5: placebo; HC/APAP; HC/APAP and 1.0 mg naltrexone (NTX); HC/APAP and 0.1 mg NTX; HC/APAP and 0.01 mg NTX; HC/APAP and 0.001 mg NTX.

FIGS. 38B and 38C show the summary of pain intensity difference (SPID) results at 4 hours (SPID-4) (see also Tables 69A and 69B) for women and men, respectively, in the six study groups as described in Example 6: placebo; HC (5 mg)/APAP (500 mg); HC/APAP and 0.001 mg naltrexone (NTX); HC/APAP and 0.01 mg NTX; HC/APAP and 0.1 mg NTX; HC/APAP and 1.0 mg NTX.

FIGS. 39A and 39B show the time to remedication (rescue medication) up to 8 hours, for women and men, respectively (see also Tables 72A and 72B) in the six study groups as described in Example 6: placebo (A); HC/APAP (B); HC/APAP and 1.0 mg naltrexone (NTX) (C); HC/APAP and 0.1 mg NTX (D); HC/APAP and 0.01 mg NTX (E); HC/APAP and 0.001 mg NTX (F)

FIGS. 40A for women and 40B for men show a summary of adverse side effects (see also Tables 77A and 77B) of nausea, vomiting, dizziness, headache, somnolence (sedation) or pruritus in the six study groups described in Example 6: placebo; HC (5 mg)/APAP (500 mg); HC/APAP and 0.001 mg naltrexone (NTX); HC/APAP and 0.01 mg NTX; HC/APAP and 0.1 mg NTX; HC/APAP and 1.0 mg NTX.

FIG. 41 shows the total pain relief (TOTPAR) results (see also Table 81) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 42 shows the summary of pain intensity difference (SPID) results at 4 hours (SPID-4) (see also Table 82) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 43 shows the probability to onset of analgesia (see also Table 43) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 44 shows the probability to remedication (rescue medication) over time up to 24 hours (see also Table 84) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 45 shows the pain relief (PR) results (see also Table 86) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 46 shows the pain intensity differences (PD) results (see also Table 87) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 47 shows the global evaluations of pain relief (see also Table 89) for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 48 shows the summary of adverse side effects (see also Table 90) of nausea, vomiting, dizziness, headache, sommolence (sedation) or pruritus for subjects in the seven study groups as described in Example 7: placebo; morphine (30 mg); morphine (30 mg) and NTX (0.1 mg); morphine (60 mg); morphine (60 mg) and NTX (0.1 mg); morphine (90 mg); morphine (90 mg) and NTX (0.1 mg).

FIG. 49 shows the day-one mean pain intensity difference (PD) results (see also Table 91) for the three intrathecal morphine study groups as described in Example 8: placebo, NTX (0.001 mg), and NTX (0.01 mg).

FIG. 50 shows the mean pain intensity difference (PID) results (see also Table 92) for days two through seven results for the three intrathecal morphine study groups as described in Example 8: placebo, NTX (0.001 mg), and NTX (0.01 mg).

FIG. 51 shows the day-one pain intensity difference (PID) results morphine study groups as described in Example 8: Tables 93A and 93B for days two through eight results for the three intrathecal placebo, NTX (0.001 mg), and NTX (0.01 mg).

FIGS. 52A and 52B show the mean hourly pain intensity difference (PID) results for women and men, respectively, in the five study groups as described in Example 9: placebo (A); tramadol and placebo (B); tramadol and 1.0 mg naltrexone (NTX) (C); tramadol and 0.1 mg NTX (D); tramadol and 0.01 mg NTX (E).

### DETAILED DESCRIPTION

The present invention is directed to novel compositions and methods with opioid agonists and opioid antagonists. Novel combinations of such agonists and antagonists were unexpectedly efficacious in enhancing the analgesic potency of the agonist without attenuating (e.g., reducing, blocking, inhibiting or preventing) the side effects of the agonist in humans, or maintaining the analgesic potency of the agonist while attenuating (e.g., reducing, blocking, inhibiting or preventing) side effects of the agonist in humans.

The present invention is based on surprising results from clinical trials that the analgesic potency effects of opioid agonists can be dissociated from their adverse effects in humans. Thus, for the first time, the present invention provides compositions and methods to differentially dose or treat humans with opioid agonists and opioid antagonists to specifically either (1) enhance (e.g., increase) analgesic potency of the opioid agonists without substantially reducing or increasing (e.g., maintain) the adverse side effects in humans associated with that dose of agonist; or (2) maintain the analgesic potency (e.g., neither substantially increase or decrease potency) of the opioid agonists while attenuating (e.g., reducing, blocking, inhibiting or preventing) the adverse side effects in humans associated with that dose of agonist. For compositions and methods of the invention that enhance analgesic potency of the opioid agonist, it is advantageous that adverse side effects are maintained or not increased with that enhanced (e.g., increased) potency. For compositions and methods of the invention that attenuate (e.g., reduce, block or prevent) the adverse side effects of the opioid agonist, it is advantageous that the analgesic potency is maintained without increasing or decreasing the cumulative daily dose of agonist.

The present invention is also directed to novel compositions of and methods using non-kappa opioid receptor agonists, preferably mu opioid receptor agonists, and opioid antagonists for gender-based dosing of the agonist and/or the antagonist in men and women. Such novel combinations of such agonists and antagonists are unexpectedly efficacious in enhancing (e.g., increasing) the analgesic potency of the agonists without enhancing the side effects of the agonists in men, and in maintaining the analgesic potency of the agonist while attenuating (e.g., reducing, blocking, inhibiting or preventing) the adverse side effects of the agonist in women.

The present invention is based on several surprising results from human clinical trials, including that (i) the analgesic potency and/or the adverse side effects of morphine sulfate, a non-kappa (mu) opioid receptor agonist is gender-specific; (ii) the effects of naltrexone, an opioid antagonist, are gender-specific, and it appears to act as a partial opioid agonist on opioid receptors in women and men, but its partial agonist effects are gender-specific; and (iii) interactions between such a non-kappa (mu) opioid receptor agonist and an opioid antagonist are gender-specific. Additionally surprising from these clinical trials is that the analgesic activity, including analgesic potency, of such non-kappa (mu) opioid receptor agonists can be dissociated from their adverse effects in humans based upon gender. Thus, for the first time, the present invention provides compositions and methods for the differential dosing of non-kappa opioid receptor agonists, preferably mu opioid receptor agonists, and/or opioid antagonists in men and women. Compositions and methods according to the invention include those that yield, for example, either (1) analgesia in men using a hypo-analgesic dose (including a non-analgesic or anti-analgesic dose) of a non-kappa opioid receptor agonist, preferably a mu opioid receptor agonist, and a dose of opioid receptor antagonist that in combination provides or enhances analgesia, thus converting non-responder human subjects (e.g. men) into responder, or (2) analgesia in women using an analgesic dose of a non-kappa opioid receptor agonist, preferably a mu opioid receptor agonist, and a dose of opioid receptor antagonist that in combination maintains the analgesia comparable to that of the against alone, but with attenuation (e.g., in number and/or severity) of one or more of the adverse side effects associated with such an agonist.

For compositions and methods of the invention that provide or enhance (e.g., increase) pain relief or attenuate (e.g., decrease) pain intensity with a non-kappa opioid receptor agonist, preferably a mu opioid receptor agonist, for example, in men, it is advantageous that the adverse side effects associated with the agonist are not enhanced with the provided or enhanced pain relief or attenuated pain intensity. For compositions and methods of the invention that enhance pain relief or attenuate pain intensity of a non-kappa opioid receptor agonist, preferably a mu opioid receptor agonist, for example, in women, it is advantageous that the adverse side effects are attenuated. For compositions and methods of the invention that attenuate the adverse side effects (e.g., in number and/or severity) of such agonists, it is advantageous that the analgesic potency be maintained while decreasing the cumulative 24 hour dose of such agonists, thus maintaining responder human subjects (e.g., women) as responders but with attenuation of one or more adverse side effects.

Compositions and methods according to the invention include those with a non-kappa opioid receptor agonist, preferably a mu opioid receptor agonist, and opioid antagonist in amounts that are useful for men only, useful for women only, or useful for both men and women, taking into account the gender-based differences described and claimed herein. Such compositions and methods are useful to provide or enhance pain relief, attenuate pain intensity, or attenuate one or more of the adverse side effects of the agonist.

It will be appreciated that compositions and methods of the invention useful for human subjects (e.g., patients) will be primarily of use in the alleviation or attenuation of established symptoms but prophylaxis is not excluded.

The term “opioid” refers to compounds or compositions including metabolites of such compounds or compositions which bind to specific opioid receptors and have agonist (activation) or antagonist (inactivation) effects at these receptors, such as opioid alkaloids, including the agonist morphine and its metabolite morphine-6-glucuronide and the antagonist naltrexone and its metabolite and opioid peptides, including enkephalins, dynorphins and endorphins. The opioid can be present as a member selected from an opioid base and an opioid pharmaceutically acceptable salt. The pharmaceutically acceptable salt embraces an inorganic or an organic salt. Representative salts include hydrobromide, hydrochloride, mucate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate, bi(pyridine-3-carboxylate), bi(trifluoroacetate), bitartrate, chlorhydrate, fumarate and sulfate pentahydrate. The term “opiate” refers to drugs derived from opium or related analogs.

An “opioid receptor agonist” or “opioid agonist” is an opioid compound or composition including any active metabolite of such compound or composition that binds to and activates opioid receptors, for example, on nociceptive neurons which mediate pain. Such agonists have analgesic activity (with measurable onset, peak, duration and/or total effect) and can produce analgesia. Opioid agonists include: alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanil, sufentanil, tramadol, tilidine, salts thereof, mixtures of any of the foregoing, mixed mu-agonists/antagonists, mu-antagonist combinations, or the like. Preferred opioid agonists for human use are morphine, hydrocodone, oxycodone, codeine, fentanyl (and its relatives), hydromorphone, meperidine, methadone, oxymorphone, propoxyphene or tramadol, or mixtures thereof. Particularly preferred opioid agonists include morphine, hydrocodone, oxycodone or tramadol. Opioid agonists include exogenous or endogenous opioids.

“Bimodally-acting opioid agonists” are opioid agonists that bind to and activate both inhibitory and excitatory opioid receptors on nociceptive neurons which mediate pain. Activation of inhibitory receptors by said agonists causes analgesia. Activation of excitatory receptors by said agonists results in anti-analgesia, hyperexcitability, hyperalgesia, as well as development of physical dependence, tolerance and other undesirable side effects. Bimodally-acting opioid agonists may be identified by measuring the opioid's effect on the action potential duration (APD) of dorsal root ganglion (DRG) neurons in tissue cultures. In this regard, bimodally-acting opioid agonists are compounds which elicit prolongation of the APD of DRG neurons at pM-nM concentrations (i.e., excitatory effects), and shortening of the APD of DRG neurons at μM concentrations (i.e., inhibitory effects).

A “non-kappa opioid receptor agonist” or “morphine-like opioid receptor agonist” is an opioid agonist that primarily binds to and/or interacts with opioid receptors that are not kappa receptors and does not produce its therapeutic effects primarily via kappa opioid receptors. Such agonists include mu, delta and sigma opioid receptor agonists and specifically exclude kappa opioid receptor agonists. Such agonists exclude, for example, agonists that primarily bind to and interact with kappa opioid receptors, and from such interactions produce their therapeutic effects (e.g., analgesic activity), such as pentazocine, nalbuphine and butorphanol. Such agonists include, for example, morphine, hydrocodone, oxycodone, codeine, hydromorphone, levorphanol, meperidine, fentanyl, (and its relatives), oxymorphone, propoxyphene, methadone or tramadol. A preferred non-kappa opioid agonist is a mu opioid receptor agonist. According to the invention, such agonists include an agonist that exhibits non-kappa gender-based effects in men and women as described and claimed herein.

A “mu opioid receptor agonist” is an opioid agonist that primarily binds to and/or interacts with mu opioid receptors and from such interactions produces its therapeutic effects (e.g., analgesic activity), such as morphine, hydrocodone, and oxycodone, but excluding agonists that primarily bind to and interact with kappa opioid receptors, and from such interactions produce their therapeutic effects (e.g. analgesic activity), such as pentazocine, nalbuphine and butorphanol.

A “delta opioid receptor agonist” is an opioid agonist that primarily binds to and/or interacts with delta opioid receptors and from such interactions produces its therapeutic effects (e.g., analgesic activity), but excluding agonists that primarily bind to and interact with kappa opioid receptors, and from such interactions produce their therapeutic effects (e.g., analgesic activity), such as pentazocine, nalbuphine and butorphanol. Selective delta opioid receptor agonists include those described by U.S. Pat. Nos. 5,389,645 and 5,985,880 hereby incorporated by reference in its entirety [e.g., a cyclic enkephalin analog [D-Pen², D-Pen⁵]-(enkephalin) and, heptapeptides of frog skin origin [deltorphin I and II] (see also U.S. Pat. No. 4,518,711 hereby incorporated by reference in its entirety)].

A “mu-delta opioid receptor agonist” is an opioid agonist that primarily binds to and/or interacts with mu and delta opioid receptors and from such interactions produces its therapeutic effects (e.g., analgesic activity), but excluding agonists that primarily bind to and interact with kappa opioid receptors, and from such interactions produce their therapeutic effects (e.g., analgesic activity), such as pentazocine, nalbuphine and butorphenal. Selective mu-delta opioid receptor agonists include those described by U.S. Pat. No. 5,389,645 hereby incorporated by reference in its entirety [e.g., tyrosyldiamine amide opioid agonists such as U.S. Pat. No. 6,054,557 hereby incorporated by reference in its entirety; U.S. Pat. No. 5,872,097 hereby incorporated by reference in its entirety; U.S. Pat. Nos. 6,568,908, 5,681,830, 5,658,908 and 5,854,249, each and all incorporated by reference in their entirety [e.g., diarylmethylpiperazines and piperidines such as 3-((αR)-α-((2S,5R)-4-allyl-2,5,-dimethyl-1-piperazinyl)-3-hydroxybenzyl)-N,N-diethylbenzamine]; and the synthetic pentapeptide known as DADLE (see, e.g., U.S. Pat. No. 5,985,600 hereby incorporated by reference in its entirety).

A “kappa opioid receptor agonist” is an opioid agonist that primarily binds to and/or interacts with kappa opioid receptors and from such interactions produces its therapeutic effects (e.g., analgesic activity), including, for example, pentazocine, nalbuphine and butorphenol. Selective kappa opioid agonists include those described by: U.S. Pat. No. 4,923,863 hereby incorporated by reference in its entirety [e.g., morpholine derivatives]; U.S. Pat. No. 6,110,947 hereby incorporated by reference in its entirety [e.g., pyrrolidinyl hydroxamic acid compounds]; U.S. Pat. No. 5,965,701 hereby incorporated by reference in its entirety [e.g., kappa receptor opioid peptides with affinity for the kappa opioid receptor at least 1,000 times greater than its affinity for the mu opioid receptor].

A “sigma opioid receptor agonist” is an opioid agonist that primarily binds to and/or interacts with sigma opioid receptors and from such interactions produces its therapeutic effects (e.g., analgesic activity), but excluding agonists that primarily bind to and interact with kappa opioid receptors, and from such interactions produce their therapeutic effects (e.g., analgesic activity), such as pentazocine, nalbuphine and butorphanol. Selective sigma opioid agonists include those described by: U.S. Pat. Nos. 5,656,633 and 5,556,857, both incorporated by reference (e.g., carbostyril derivatives).

An “opioid antagonist” is an opioid compound or composition including any active metabolite of such compound or composition that in a sufficient amount attenuates (e.g., blocks, inhibits, or competes with) the action of an opioid agonist. An “effective antagonistic” amount is one which effectively attenuates the analgesic activity of an opioid agonist. An opioid antagonist binds to and blocks (e.g., inhibits) opioid receptors, for example, on nociceptive neurons which mediate pain. Opioid antagonists according to the present invention include: naltrexone, naloxone nalmefene, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, nalorphine, nalbuphine, thebaine, levallorphan, oxymorphone, butorphanol, buprenorphine, levorphanol, meptazinol, pentazocine, dezocine, or their pharmacologically effective esters or salts. An opioid antagonist with partial agonist activity is cholera toxin B. Preferred opioid antagonists include naltrexone, nalmefene, naloxone, or mixtures thereof. Particularly preferred antagonists include naltrexone and nalmefene. Naltrexone as a most preferred opioid antagonist.

“Excitatory opioid receptor antagonists” are opioids which bind to and act as antagonists to excitatory but not inhibitory opioid receptors on nociceptive neurons which mediate pain. That is, excitatory opioid receptor antagonists are compounds which bind to excitatory opioid receptors and selectively block excitatory opioid receptor functions of nociceptive types of DRG neurons at 1,000 to 10.000-fold lower concentrations than are required to block inhibitory opioid receptor functions in these neurons. Excitatory opioid receptor antagonists may also be identified by measuring their effect on the action potential duration (APD) of dorsal root ganglion (DRG) neurons in tissue cultures. In this regard, excitatory opioid receptor antagonists are compounds which selectively block prolongation of the APD of DRG neurons (i.e., excitatory effects) but not the shortening of the APD of DRG neurons (i.e., inhibitory effects) elicited by a bimodally-acting opioid receptor agonist. Preferred excitatory opioid receptor antagonists are naltrexone and nalmefene because of their longer duration of action as compared to naloxone and their greater bioavailability after oral administration.

Other compounds and compositions of opioid agonists, including non-kappa opioid receptor agonists, preferably mu opioid receptor agonists, and opioid antagonists are known and will be readily apparent to those skilled in the art, once armed with the present disclosure.

The opioid agonists or opioid antagonists may be provided in the form of free bases or pharmaceutically acceptable acid addition salts. As used herein, “pharmaceutically acceptable salts” refer to derivatives of the disclosed compounds wherein the therapeutic compound is modified by making acid or base salts thereof. The pharmaceutically acceptable salt embraces an inorganic or an organic salt.

Examples of pharmaceutically acceptable salts include, but are not limited to, mineral or organic acid salts of the opioid antagonist or opioid agonist. The pharmaceutically acceptable salts include the conventional non-toxic salts made, for example, from non-toxic inorganic or organic acids. For example, such conventional non-toxic salts include those derived from inorganic acids such as hydrochloric, hydrobromic, sulfuric, sulfonic, sulfamic, phosphoric, nitric and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucoronic, and other acids. Other pharmaceutically acceptable salts and variants include mucates, phosphate (dibasic), phosphate (monobasic), acetate trihydrate, bi(heptafluorobutyrate), bi(methylcarbamate), bi(pentafluoropropionate), mesylate, bi(pyridine-3-carboxylate), bi(trifluoroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ration.

An “adverse side effect” of an opioid agonist is a side effect in humans, typically associated with opioid analgesics such as morphine, including nausea, vomiting, dizziness, somnolence/sedation, pruritus, reduced gastrointestinal mortality including constipation, difficulty in urination, peripheral vasodilation including leading to orthostatic hypotension, headache, dry mouth, sweating, asthenia, dependence, mood changes (e.g., dysphoria, euphoria), or lightheadedness. An “adverse side effect” also includes a serious adverse side effect such as respiratory depression or also apnea, respiratory arrest, circulatory depression, hypotension or shock.

As demonstrated herein, opioid agonists may produce certain adverse side effects. Among the side effects that have been recognized for products containing morphine or other opioid agonists are: respiratory depression; depression of the cough reflex; miosis; reduced gastrointestinal motility including constipation; peripheral vasodilation which may result in orthostatic hypotension; and release of histamine. Adverse side effects that are of particular interest in human subjects include nausea, vomiting, dizziness, headache, somnolence (sedation), and pruritus. Some additional adverse side effects are listed in the Physician Desk Reference (PDR) for selected opioid agonists as follows: morphine: respiratory depression; apnea; circulatory depression; shock respiratory arrest, and cardiac arrest; oxycodone: light-headedness, euphoria, dysphoria, constipation, skin rash; hydrocodone: mental clouding, lethargy, impairment of mental and physical performance, anxiety, fear, dysphoria, dependence, mood changes; constipation; ureteral spasm; spasm of vesical sphincter and urinary retention; and tramadol: seizures; anaphylactoid reactions (lessened resistance to toxins); asthenia; sweating; dyspepsia; dry mouth; diarrhea; CNS stimulation (“CNS stimulation” is a composite that can include nervousness, anxiety, agitation, tremor, spasticity, euphoria, emotional liability and hallucinations); malaise; vasodilation; anxiety, confusion, coordination disturbance, euphoria, nervousness, sleep disorder; abdominal pain, anorexia, flatulence, hypertonia, rash, visual disturbance, menopausal symptoms, urinary frequency, urinary retention.

“Co-administer,” “co-administration,” “concurrent administration” or “co-treatment” refers to administration of an opioid agonist and an opioid antagonist, in conjunction or combination, together, or before or after each other. The opioid agonist and the opioid antagonist may be administered by different routes. For example, the agonist may be administered orally and the antagonist intravenously, or vice versa. The opioid agonist and opioid antagonist are preferably both administered orally, as immediate or sustained release formulations. The opioid agonist and opioid antagonist may be administered simultaneously or sequentially, as long as they are given in a manner to allow both agents to achieve effective concentrations to yield their desirable therapeutic effects (e.g., analgesia). Optionally, an additional active pharmaceutical ingredient may be co-administered with the opioid agonist and opioid antagonist. For example, other active pharmaceutical ingredients include acetaminophen as shown herein, steroidal drugs or non-steroidal anti-inflammatory drugs (NSAIDS) such as ibuprofen, COX-1 and/or COX-2 inhibitors such as aspirin, rofecoxib (marketed as VIOXX®), and celcoxib (marketed as CELEBREX™).

“Combination” refers to more than one active compound or active pharmaceutical ingredient (API), including for example, a combination of opioid agonist and opioid antagonist.

“Therapeutic effect” or “therapeutically effective” refers to an effect or effectiveness that is desirable and that is an intended effect associated with the administration of an opioid agonist including the opioid agonist in combination with an opioid antagonist according to the invention, including, for example, analgesia, pain relief, decrease in pain intensity, euphoria or feeling good or calming so as to reduce heart rate, blood pressure or breathing rate.

The opioid agonists preferably and the opioid antagonists for use in the present invention may be in the form of free bases or pharmaceutically acceptable acid addition salts thereof.

The opioid antagonist alone, or in combination with the opioid agonist, may be administered to the human subject by known procedures including but not limited to oral, sublingual, transmucosal (including buccal), intramuscular, subcutaneous, intravenous, intratracheal, or transdermal modes of administration. When a combination of these compounds are administered, they may be administered together in the same composition, or may be administered in separate compositions. If the opioid agonist and the opioid antagonist are administered in separate compositions, they may be administered by similar or different modes of administration, or may be administered simultaneously with one another, or shortly before or after the other.

The opioid agonists and the opioid antagonists may be formulated in compositions with a pharmaceutically acceptable carrier. The carrier must be “acceptable” in the sense of being compatible with the other ingredients of the formulation and not deleterious to the recipient thereof. Examples of suitable pharmaceutical carriers include lactose, sucrose, starch, talc, magnesium stearate, crystalline cellulose, methyl cellulose, carboxymethyl cellulose, glycerin, sodium alginate, gum arabic, powders, saline, water, among others. The formulations may conveniently be presented in unit dosage and may be prepared by methods well-known in the pharmaceutical art, by bringing the active compound into association with a carrier or diluent, as a suspension or solution, or optionally with one or more accessory ingredients, e.g., buffers, flavoring agents, surface active agents, or the like. The choice of carrier will depend upon the route of administration. “Unit dose form” or “unit dosage form” refers to physically discreet units suitable as unitary doses for human subjects, each unit containing a predetermined quantity of active material (e.g., non-kappa opioid receptor agonist and/or opiold antagonist and/or other active pharmaceutical ingredient) calculated to produce the desired therapeutic effect (e.g. analgesia), in association with a suitable pharmaceutical carrier. Thus, the active ingredients according to the invention (e.g., agonist, antagonist, or other active pharmaceutical ingredient) either each alone or in combination may conveniently be presented to the subject for administration in unit dose form.

For oral or sublingual administration, including transmucosal, the formulation may be presented as capsules, tablets, caplets, pills, powders, granules or a suspension, prepared by conventional means with pharmaceutically acceptable excipients, e.g., with conventional additives or fillers such as lactose, mannitol, corn starch or potato starch; with binders or binding agents such as crystalline cellulose, cellulose derivatives, acacia, corn starch (including pregelatinized) or gelatins; with disintegrators or disintegrants such as corn starch, potato starch or sodium carboxymethyl-cellulose; or with lubricants or wetting agents such as talc or magnesium stearate. Tablets may be coated, including by methods well known in the art. The formulation may be presented as an immediate-release or as a slow-release, sustained-release or controlled-release form. The formulation may also be presented as a solid drug matrix, for example, on a handle. Oral dose forms for human administration include: codeine, dihydrocodeine (e.g., SYNALGOS-DC® from Wyeth-Ayerst Pharmaceuticals), fentanyl (e.g., ACTIQ® from Abbott Laboratories)., hydrocodone (e.g., VICODIN® and VICOPROFEN® from Knoll Laboratories; NORCO® from Watson Laboratories; HYCODAN® from Endo Pharmaceuticals; NORCET® from Abara; ANEXSIA®, HYDROCET®, and LORCET-HD® from Mallinckrodt; LORTAB® from UCB Pharma; HY-PHEN® from Ascher; CO-GESIC® from Schwarz Pharma; ALLAY® from Zenith Goldline), hydromorphone (e.g. DILAUDID® from Knoll), levorphanol (e.g., LEVO-DROMORAN® from ICN Pharmaceuticals), meperidine (e.g., DEMEROL® from Sanofi Pharmaceuticals), methadone (e.g., METHADOSE® from Mallinckrodt; and DOLOPHINE® HCl from Roxane Laboratories), morphine (e.g., KADIAN® from Faulding Laboratories; MS CONTIN® from Purdue Frederick; ORAMORPH® SR from Roxane), oxycodone (e.g., PERCOCET® and PERCODAN® from Endo; OXYCET® from Mallinckrodt; OXYCONTIN® from Purdue Frederick; TYLOX® from Ortho-McNeil Pharmaceutical; ROXICODONE®, ROXILOX® and ROXICET® from Roxane), pentazocine (e.g., TALACEN® and TALWIN® from Sanofi Pharmaceuticals), propoxyphene (e.g., DARVOCET-N® and DARVON®from Eli Lilly & Co.; DOLENE® from Lederle; WYGESIC® from Wyeth-Ayerst), and tramadol (e.g., ULTRAM® from Ortho-McNeil Pharmaceutical).

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., methyl or propyl-p-hydroxybenzoates or sorbic acid). Liquid dose forms for human administration include: hydrocodone (e.g., HYDROPHANE® from Halsey), hydromorphone (e.g., DILAUDID® from Knoll), meperidine (e.g., DEMEROL® from Sanofi), methadone (e.g., DOLOPHINE® from Roxane), oxycodone (e.g., HYCOMINE® from Knoll; ROXILOX® from Roxane), and propoxyphene (e.g., DARVON-N® from Eli Lilly).

For parenteral administration, including intravenous, intramuscular, or subcutaneous administration, the compounds may be combined with a sterile aqueous solution which is preferably isotonic with the blood of the recipient. Such formulations may be prepared by dissolving solid active ingredient in water containing physiologically compatible substances such as sodium chloride, glycine, or the like, and/or having a buffered pH compatible with physiological conditions to produce an aqueous solution, and/or rendering said solution sterile. The formulations may be present in unit dose forms or multi-dose forms, including in containers such as sealed ampoules or vials. Parenteral dose forms for human administration include: alfentanil (e.g., ALFENTA® from Akorn), buprenorphine (e.g., BUPRENEX® from Reckitt & Colman Pharmaceuticals), butorphanol (e.g., STADOL® from Apothecon), dezocine (e.g., DALGAN® from Astrazeneca), fentanyl, hydromorphone (e.g., DILAUDID-HP® from Knoll), levallorphan (e.g., LORFAN® from Roche), levorphanol (e.g., LEVO-DROMORAN®from ICN), meperidine (e.g., DEMEROL® from Sanofi), methadone (e.g., DOLOPHINE® HCl from Roxane), morphine (e.g., ASTRAMORPH® from Astrazeneca; DURAMORPH® and INFUMORPH® from Elkins-Sinn), oxymorphone (e.g., NUMORPHAN® from Endo), nalburphine (e.g., NUBAIN® from Endo Pharmaceutical), and pentazocine (TALWIN® from Abbott).

For transdermal administration, the compounds may be combined with skin penetration enhancers such as propylene glycol, polyethylene glycol, isopropanol, ethanol, oleic acid, N-methylpyrrolidone, or the like, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, polyvinyl pyrrolidone, or the like, to provide the composition in gel form, which can be dissolved in solvent such as methylene chloride, evaporated to the desired viscosity, and then applied to backing material to provide a patch. Transdermal dose forms for human administration include fentanyl (e.g., DURAGESIC® from Janssen).

Additional dose forms available as suppositories for human administration include oxymorphone (e.g. NUMORPHAN® from Endo).

“Analgesia” refers to the attenuation, reduction or absence of sensibility to pain, including the provision of pain relief, the enhancement of pain relief, or the attenuation of pain intensity. An “analgesic” amount refers to an amount of the opioid agonist which causes analgesia in a subject administered the opioid agonist alone, and includes standard doses of the agonist which are typically administered to cause analgesia (e.g., mg doses). An “analgesic” amount also refers to an amount that results in analgesic efficacy, for example, as measured by a female or male subject with a pain relief score or a pain intensity difference score, at a given time point, or over time, or as compared to a baseline, and includes calculations based on area under the curve such as TOTPAR or SPID from such pain relief scores or pain intensity difference scores. A “hypo-analgesic” amount is a less-than-analgesic amount, including an amount which is not analgesic or is weakly analgesic in a subject administered the opioid agonist alone, and further includes an “anti-analgesic” or “algesic” amount which is an amount which increases pain. For example, men or women in the opioid antagonist may be administered in an amount effective to provide or enhance the analgesic potency (e.g., as measured by pain relief or pain intensity difference) of the opioid agonist, without substantially increasing (e.g., maintaining) the adverse side effects as compared to the agonist alone. For example, in women or men, the opioid antagonist may be administered in an amount effective to maintain the analgesic potency (e.g., maintain analgesia as measured by pain relief or pain intensity differences) of the opioid against, while attenuating one or more adverse side effects of the agonist. The opioid antagonist may be administered in an amount effective to produce or enhance analgesic potency in combination with, for example, a mu opioid receptor agonist. The optimum amounts, for example, of the opioid agonist and the opioid antagonist administered, will of course depend upon the particular agonist and antagonist used, the carrier chosen, the route of administration, and/or the pharmacokinetic properties of the subject being treated, as well as the desired gender-related effects according to the teachings of the present invention. When the opioid antagonist is administered alone, the amount of the opioid antagonist administered is an amount effective to enhance or maintain the analgesic potency of the opioid agonist and/or attenuate or maintain the adverse side effects of the opioid agonist, according to the teachings of the present invention.

Examples 1-9 that follow, describe in detail, results from human clinical trials, including those with a retrospective or prospective gender analysis, that unexpectedly demonstrate that the responses to opioid agonists such as morphine, hydrocodone, or tramadol and the responses to naltrexone, an opioid antagonist, as well as the responses to the interactions between such an agonist and antagonist, show surprising effects in humans, including surprising clinical benefits from the combination of such agonists and antagonists. Such clinical benefits include enhancing the potency (e.g., increasing pain relief or decreasing pain intensity in humans) of a dose of the opioid agonist, while maintaining the adverse side effects of the agonist at that dose or maintaining the potency of a dose of the opioid agonist while attenuating (e.g., reducing, blocking, inhibiting or preventing) one or more adverse side effects in humans associated with that dose of agonist. The responses to non-kappa opioid receptor agonists, such as morphine, hydrocodone or tramadol are strikingly different in women and men. By way of example, Examples 1-4 and 7 describe data that have been collected from observations in populations of human patients, wherein males and/or females were subjected to painful stimulation during the course of dental extractions and then treated with naltrexone and/or morphine. In Examples 1 and 2, subjects had two or more impacted third molars requiring extraction, wherein at least one extracted tooth was a partial or full bony mandibular impaction. In Examples 3-4 and 7, subjects had three or four full or partial bony impacted third molars requiring extraction. The levels of pain experienced by the subjects, for example, those in Examples 3-4, are not explicable by the known activity of naltrexone as a pure antagonist of morphine on nociceptive pathways. Data presented herein relate to novel gender-based differences and the data are consistent with a mechanism whereby an opioid antagonist such as naltrexone can act as a partial agonist on opioid receptors that are responsive to an opioid agonist such as morphine.

The studies demonstrate a number of gender-related differences, first with respect to the responses of the female and male subjects to the antagonist alone. For example, in females, naltrexone, by itself, acts as a hypo-analgesic agent in that it can cause increased pain in subjects experiencing pain associated with the dental extractions studied. Data from a study are described in Examples 3 and 4 in which female subjects were given an oral dose of 0.01 mg naltrexone. Pain scores were determined as pain intensity differences (PID). A PID score of 0 means no change in the level of pain, whereas a negative PID score means that pain increased, and a positive PD score indicates analgesia. Within 15 minutes, the PID score in the female subjects decreased below 0, indicating that the subjects experienced increased pain. The response to naltrexone was characterized by three features. First, there was a rapid increase in pain (anti-analgesia), with a peak in pain score of less than −0.3 observed at about 45 minutes after administration of the naloxone. Thereafter, there was a slight attenuation of the pain score (rebound), which lasted about 2 hours, and thereafter, the pain score increased (late phase anti-analgesia) and remained approximately steady (PID score of about −0.3) for the duration of the study (3 hours). In contrast to the results observed for females, naltrexone given to males in the same study had no anti-analgesic or analgesic effects. Data from this study are also shown in Examples 3 and 4 in which males undergoing dental extractions were given an oral dose of 0.01 mg naltrexone. Naltrexone did not change the PID score, which remained at about 0 for the duration of the 8 hours of the study. Thus, there was no rapid anti-analgesia, rebound, or late phase anti-analgesia as observed for the female patients.

Gender-related differences were also observed in the female and male subjects with respect to the agonist alone. As with the responses to the opioid antagonist naltrexone, the responses to the opioid agonist morphine differed unexpectedly between female and male patients. For example, the results from this study as described in Examples 3 and 4 of the responses of females given an oral dose of 60 mg morphine, show that the time course of the response to morphine was slower than the time course of the response to naltrexone, with little or no effect observed at 30 minutes after administration. However, by 60 minutes, substantial analgesia was observed, as indicated by a PD score of greater than about 0.4. A broad peak in analgesia was observed between about 1.5 and about 5 hours, with the PID score remaining at or above about 0.6 for this time period. Thereafter, the PD score slowly fell, and by about 6 hours, the PID score was at about 0.5. The PD remained at about 0.5 for the duration of the study. In another study of female patients as described in Examples 1 and 2, a 60 mg oral dose of morphine was associated with progressive analgesia. In striking contrast to the results observed for females, in the males the same dose of morphine did not cause any analgesia. In fact, quite unexpectedly, morphine increased the pain that the men experienced (anti-analgesia). Within the first 15 minutes, the PID score began to fall below 0, indicating that pain was increased compared to the baseline. PID decreased to a minimum at about 45 minutes, with the PID score being about −0.2. Thereafter, the PID score slowly rose, so that by about 4 hours, the PID score had returned to about 0, where it remained for the duration of the study. In this study of male patients as described in Examples 1 and 2, morphine did cause some analgesia, but the analgesia observed was preceded by a period of anti-analgesia.

Gender-related differences were observed in the female and male subjects with respect to combinations of agonist and antagonist, in addition to the differences described above between males and females in the response to naltrexone and morphine individually. For example, in female patients (Examples 3 and 4), the combination of naltrexone and morphine at certain times and at certain concentrations caused a decrease in analgesia as compared with morphine alone. At two hours, the lowest dose of naltrexone (0.001 mg) administered in combination with morphine decreased the PID score produced in the presence of morphine from a peak of about 0.7, to about 0.4. However, by 5 hours and thereafter, naltrexone did not decrease the PD score compared to those for morphine over the same time period. Increasing the dose of naltrexone to 0.01 mg with the morphine produced somewhat more reduction in PID than did the lowest combination dose (0.001 mg). However, further increasing the dose of naltrexone to 0.1 mg produced no further decrease in PD score. Thus, the dose of naltrexone having maximal effect in females when administered with 60 mg morphine is about 0.01 mg. In another study in female patients (Examples 1 and 2), naltrexone at doses of 0.01 mg and 0.1 mg each potentiated the analgesia associated with morphine (60 mg). Further increasing the dose of naltrexone to 1.0 mg however, decreased the analgesia associated with morphine. In male patients, in the study as described in Examples 3 and 4, the lowest dose of naltrexone (0.001 mg) increased analgesia in the presence of 60 mg morphine. The increase in analgesia was moderate, with an initial analgesic effect observed by about 2 hours after administration. Increasing the dose of naltrexone to 0.01 mg increased the analgesic effect compared to the lowest dose, and further increasing the dose of naltrexone (0.1 mg) increased the analgesia further, with a substantial effect occurring at about 1 hour, and reaching a broad plateau at about 2 hours, and lasting for the duration of the study. The PID score during this time was greater than about 0.8, with several points above about 0.9. In another study in male patients as described in Examples 1 and 2, naltrexone in combination with morphine produced more analgesia than did morphine alone. The effect of naltrexone was dose-dependent with the highest doses (1.0 mg) having the greatest effect.

As shown herein, gender-related differences were observed in the female and male subjects with respect to combinations of agonist and antagonist, for example, as shown by pain relief (PR) scores, pain intensity difference scores, or adverse side effects for female and male patients, respectively, as described herein in Examples.

Gender-based opioid compositions according to the invention may have therapeutic advantages. For example, females can exhibit significant analgesic responses to an opioid agonist such as morphine, and at certain doses, an opioid antagonist such as naltrexone can potentiate the analgesia induced by morphine. However, effective doses of an opioid agonist such as morphine may have undesirable adverse side effects, including nausea, vomiting, other gastrointestinal symptoms, and other serious side effects such as respiratory depression. Additionally, an opioid antagonist such as naltrexone by itself may increase pain in females experiencing pain.

In certain embodiments of the invention, compositions are provided for use in females comprising low concentrations of opioid agonists including, by way of example only, morphine or oxycodone, that by themselves may not produce a desired degree of analgesia, along with doses of naltrexone that are sufficiently low to avoid producing undesirable adverse side effects themselves. By selecting doses of opioid agonist and antagonist, it is now possible to maintain a desirable therapeutic effect such as pain relief, while attenuating undesirable adverse side effects, for example, in females and/or males.

In certain other embodiments of this invention, compositions are provided for use in males comprising concentrations of morphine or other opioid agonists that alone are ineffective, along with naltrexone or other opioid antagonists in doses sufficient to potentiate or enhance the analgesic effects of the opioid agonist such as morphine. Additionally, because an opioid antagonist such as naltrexone can substantially potentiate or enhance the effects of an opioid agonist such as morphine, it is now possible to reduce the dose of an opioid agonist such as morphine to well below those doses that cause undesirable side effects, while at the same time, providing substantial pain relief, for example, in females and/or males.

Novel pharmaceutical compositions and dosage forms of opioid antagonists are described in U.S. Provisional Application No. 60/202,227, incorporated by reference herein. Novel compositions and gender-based methods for enhancing potency or reducing adverse side effects of opioid agonists are described in U.S. Provisional Application Nos. 60/244,482, 60/245,110, and 60/246,235, incorporated by reference herein. Additional human clinical study results with tramadol are described in U.S. application Ser. Nos. 09/566,071 and 09/756,331 as well as PCT/US00/12493 [WO00/67739], that are all incorporated by reference herein.

The present invention is described in the following examples which are set forth to aid in the understanding of the invention, and should not be construed to limit in any way the invention as defined in the claims which follow thereafter. Pharmaceutical active and inactive ingredients used in the preparation of the example formulations were compendial in the USP/NF, when there was an existing monograph.

In the following examples, encapsulated dose forms of naltrexone HCl (NTX) and various opioid agonists were prepared for clinical studies as follows. Encapsulated dose forms of naltrexone HCl were produced in the following doses and weight concentrations.

| Naltrexone HCl | | Naltrexone HCl Active Capsule |
|---|---|---|
| Capsule Dose | | Blend Concentration (% w/w) |
| 1.0 | mg | 0.3% |
| 0.1 | mg | 0.03% |
| 0.01 | mg | 0.003% |
| 0.001 | mg | 0.0003% |

A batch of NTX, 0.3% w/w blend was made by first adding naltrexone HCl and other inactive components (e.g., magnesium stearate and microcrystalline cellulose) into a planetary mixer. The inactive components were added in portion-wise steps with mixing between each addition to achieve uniformity of the NTX. The intermediate active blend was transferred from the planetary mixer to a double-cone blender.

An amount of preblended inactive components was used to rinse the planetary mixer. The rinsings were added to the double-cone blender to achieve quantitative recovery of naltrexone HCl. The remaining balance of preblended inactive components were added in portion-wise steps to the double cone blender containing the in-process material. The resulting intermediate and final mixtures were blended for an appropriate time to achieve uniformity.

Less potent formulated blends of naltrexone HCl (e.g., 0.03% w/w/, 0.003% w/w, and 0.0003% w/w) were prepared from the 0.3% w/w blend by serial dilution with the inactive components. A premeasured portion of the more concentrated active blend were added to the double cone blender. A measured amount of the preblended inactive components was added to achieve the desired dilution. The inactive blend was added in portion-wise steps to the double cone blender, with interim mixing to achieve uniformity. The NTX blends were filled into hard gelatin capsules at a controlled weight to achieve the desired unit dose of NTX.

Encapsulated dose forms of opioid agonists were prepared for clinical studies employing the same inactive components and hard gelatin capsule. Encapsulated dose forms of morphine were prepared from commercially obtained tablets (Roxane), which contained 15 mg morphine sulfate pentahydrate and various inactive components. A 60 mg morphine sulfate strength capsule was made by mixing (e.g., microcrystalline cellulose and magnesium stearate) to form a blend, and this blend and four morphine sulfate tablets were loaded into a hard gelatin capsule shell to obtain a capsule for clinical studies. Encapsulated dose forms of tramadol were prepared from commercially obtained ULTRAM® tablets (Ortho-McNeil), which contained 50 mg tramadol hydrochloride and various inactive components. A 50 mg tramadol hydrochloride strength capsule was made by mixing inactive components (e.g., microcrystalline cellulose and magnesium stearate) to form a blend, and this blend and one ULTRAM®, immediate release tablet were loaded into a hard gelatin capsule shell to obtain a capsule for clinical studies. Encapsulated dose forms of hydrocodone were prepared from commercially obtained tablets immediate release HYDROCET® capsules (Carnrick Laboratories), which contained hydrocodone bitartrate (5 mg) with acetaminophen (500 mg) and various inactive components. A 5 mg hydrocodone bitartrate/500 mg acetaminophen strength clinical capsule was made from the commercially obtained HYDROCET® capsules in the following manner. The average weight of 20 HYDROCET® capsules was determined, and the hydrocodone/acetaminophen blend contained in a predetermined number of HYDROCET® capsules was emptied into a clean bowl. The total weight of hydrocodone/acetaminophen blend needed to fill the clinical capsules with the same average weight (including 1% overage) was transferred to a capsule machine. The capsule machine filled clinical capsule shells with the hydrocodone/acetaminophen blend.

### EXAMPLE 1

A clinical study was designed as follows: (1) to compare the analgesic activity (onset, peak, duration, and total effect) of three different doses of NTX in combination with MS 60 mg versus MS 60 mg alone in subjects with moderate to severe pain in a postsurgical dental pain model to determine whether NTX enhances the analgesic effect of MS 60 mg; and (2) to evaluate the safety of three different doses of NTX in combination with MS 60 mg versus MS 60 mg alone in subjects with moderate to severe pain in a postsurgical dental pain model to determine whether the addition of NTX reduces the frequency or severity of morphine-related side effects.

Additional objectives of the study included: (1) to compare the analgesic efficacy of MS 60 mg to placebo to establish the assay sensitivity of the study; (2) to compare the analgesic activity (onset, peak, duration, and total effect) of three different doses of NTX in combination with MS 60 mg versus placebo in subjects with moderate to severe pain in a postsurgical dental pain model; and (3) to evaluate the safety of three different doses of NTX in combination with MS 60 mg versus placebo in subjects with moderate to severe pain in a postsurgical dental pain model.

A randomized, double-blind, placebo- and active-controlled, single-dose study was thus designed. There were five treatment groups: three test products, a positive control (MS 60 mg), and a negative control (placebo). Separation of placebo and MS 60 mg were used to determine the assay sensitivity of the study. The active control (MS 60 mg) was used to determine the sensitivity of the clinical endpoints. Placebo was used to control for factors not related to drug treatment. The test products were MS 60 mg with naltrexone (NTX) 1 mg, MS 60 mg with NTX 0.1 mg, and MS 60 mg with NTX 0.01 mg. A single oral dose of one of the treatments was administered when the subject was suffering moderate to severe postoperative pain. The observation period for efficacy was eight hours post treatment. The observation period for safety was 24 hours post treatment.

The Study Population was two hundred male and female outpatients with moderate to severe pain and a pain intensity score of at least 50 mm on the 100 mm Visual Analog Scale (VAS) following extraction of two or more impacted third molars. All subjects remained in the study facility for the eight-hour duration of the single-dose evaluation and then were permitted to leave the study site.

Inclusion criteria were as follows:

(1) subjects with two or more impacted third molars requiring extraction and considered to have had surgery significant enough to warrant an opioid analgesic, where at least one extracted tooth was a partial or full bony mandibular impaction;

(2) subjects willing and able to complete the pain evaluations;

(3) subjects at least 16 years of age, and if the subject was less than age 18, the subject was emancipated, or the parent or guardian gave written consent.

(4) female subjects were postmenopausal, or physically incapable of child bearing, or practicing an acceptable method of birth control (IUD, hormones, diaphragm with spermicide, condoms with spermicide, or abstinence), and if practicing an acceptable method of birth control, must also have maintained her normal menstrual pattern for the three months prior to study entry and have had a negative urine pregnancy test performed at screening and immediately prior to surgery;

(5) subjects in generally good health;

(6) subjects able to speak and understand English and provide meaningful written informed consent;

(7) subjects able to remain at die study site for the entire eight-hour study period;

(8) subjects had an initial pain intensity score of at least 50 mm on a 100 mm visual analog scale and must also describe the initial pain as moderate or severe on a four-point categorical scale; and

(9) subjects willing and able to return to the study site for the post treatment visit five to nine days after surgery.

Exclusion criteria for subjects were as follows:

(1) pregnant or breast feeding;

(2) have known allergy or significant reaction to opioids or opioid antagonists;

(3) history of chronic opioid use or opioid abuse within six months prior to study.

(4) have participated in a study of an investigational drug or device within 30 days prior to this study;

(5) have taken any of the following drugs within four hours prior to dosing: analgesics, including aspirin, acetaminophen, nonsteroidal anti-inflammatory drugs (NSAIDS), opioids, and opioid combinations, minor tranquilizers, muscle relaxants and antihistamines, where exempted from this prohibition were midazolam (Versed), lidocaine (with or without epinephrine), mepivacaine, nitrous oxide, and propofol (Diprivan) given during surgery;

(6) have taken a long-acting analgesic (e.g., long-acting NSAIDS) within 12 hours prior to this study;

(7) have taken monoamine oxidase inhibitors or tricyclic antidepressant drugs within four weeks prior to study medication;

(8) have taken serotonin reuptake inhibitors (SSRI) or St. John's wort within four weeks prior to the study unless the subject has been on a stable dose for at least six weeks and the stable dose for St. John's wort must have been no more than 1 gm/day;

(9) have a medical or psychiatric condition that compromises the subject's ability to give informed consent or appropriately complete the pain assessments; and

(10) have a history of seizure, however, subjects with a history of juvenile febrile seizures could be included if there was no seizure history within the past 10 years.

Subjects were assigned to treatment groups based on a randomization schedule prepared prior to the study. The randomization was balanced by using equally balanced blocks. Based on the randomization code, the assigned study drug was packaged and labelled for each subject. Subject numbers were preprinted onto the study drug labels and assigned as subjects qualified for the study and were randomized to treatment. In order to achieve balance among treatment groups with respect to starting pain, the study stratified randomization according to initial pain intensity. Subjects with moderate starting pain were assigned medication with the lowest available number. Subjects with severe starting pain were assigned medication with the highest available number.

Each subject was assigned one bottle containing two capsules. The label on the bottle consisted of two parts. One part was attached firmly to the bottle and did not contain drug identification. The other part was a tear-off label containing the concealed drug identification. The tear-off label was taped unopened onto the case report form.

NUMBER OF CAPSULES PER BOTTLE FOR EACH
TREATMENT GROUP

| | | Capsules | | | | |
|---|---|---|---|---|---|---|
| Treatment | Contents | MS | NTX | NTX | NTX | |
| Group | Treatment | 60 mg | 1 mg | 0.1 mg | 0.01 mg | Placebo |
| Group A | Placebo | 0 | 0 | 0 | 0 | 2 |
| Group B | MS 60 mg | 1 | 0 | 0 | 0 | 1 |
| Group C | MS 60 mg with | 1 | 0 | 0 | 1 | 0 |
| | NTX 0.01 mg | | | | | |
| Group D | MS 60 mg with | 1 | 0 | 1 | 0 | 0 |
| | NTX 0.1 mg | | | | | |
| Group E | MS 60 mg with | 1 | 1 | 0 | 0 | 0 |
| | NTX 1 mg | | | | | |

Included on the open portion of the label was the protocol identification, subject number, number of capsules, directions for use, storage instructions, and cautionary statement about investigational status.

The randomization code was not revealed to study subjects, investigators, clinical staff or study monitors until all subjects completed therapy and the data base has been finalized and closed.

Following washout from previous analgesia as stated in the exclusion criteria, and following a suitable recovery from anesthesia after surgery, all subjects who had moderate to severe pain and a score of at least 50 mm on the 100 mm VAS received one dose of study medication, consisting of two capsules. There was one bottle per subject, labeled by subject number, as described above.

The following screening procedures were accomplished within 14 days prior to surgery: (a) review of inclusion and exclusion criteria; (b) informed consent; (c) urine pregnancy test for women of child-bearing potential (at screening and immediately prior to surgery); (d) medical history and demographics; (e) brief physical examination; and (t) vital signs.

Baseline measurements and procedures included: (a) vital signs (prior to dosing); (b) review of medications received within 12 hours prior to dosing; and (c) after a suitable washout period from the anesthesia, the subject's pain level was assessed by a trained observer, and when the pain level was moderate or severe, and the score on the 100 mm VAS was at least 50 mm, the subject was randomized to a treatment group.

Provided the subject met the above-referenced criteria, the subject was assigned the next sequential treatment number in ascending or descending order depending upon the starting pain. The subject then took one dose of study medication consisting of two capsules.

Treatment period procedures and measurements included:

(a) Following dosing, the subject remained at the study facility for eight hours;

(b) Two stopwatches were started at the time the study medication was taken at baseline and each subject was first instructed, “Stop the first stopwatch when you first feel any paid relief whatsoever. This does not mean you feel completely better, although you might, but when you first feel any difference in the pain you have now.” and then the subject was instructed, “Stop the second stopwatch when the pain relief is meaningful to you.”;

(c) For treated subjects, vital signs were taken one hour after dosing and at the end of the eight-hour observation period;

(d) For treated subjects, pain intensity and pain relief were measured by a trained observer at the following times: 30 minutes, 60 minutes and hourly thereafter through Hour 8 after dosing, and all efficacy assessments were recorded by the subject in a diary in response to questioning by a trained observer, wherein the trained observer questioned the subject for all observations and provided instruction as needed; pain intensity was measured in response to the question, “What is your pain level at this time?” with subject response choices of none=0, mile=1, moderate=2 and severe=3 on a categorical scale and the pain relief relative to baseline was assessed in response to the question, “How much relief have you had from your starting pain?” with subject response choices of none=0, a little=1, some=2, a lot=3, and complete=4;

(e) Subjects not completing at least 90 minutes after dosing were considered not evaluable and were replaced;

(f) Adverse events were assessed by non-directed questioning and recorded for the eight hours following dosing;

(g) All concomitant medications (including rescue medications) were recorded for the eight-hour observation period;

(h) At the end of eight hours, or at the termination of hourly observations if sooner than eight hours, a global evaluation was made by observer and subject in response to the question, “How do you rate the pain relief?” with response choices of poor=0, fair-=1, good=2, very good=3 and excellent=4; and

(i) Upon discharge from the study facility, the subject was given a diary to take home for recording medications taken and adverse events experienced from the time of discharge until 24 hours after the time of dosing with study medication; a member of the study staff telephoned the patient 24 hours after the time of dosing to query the subject about medications taken, adverse events experienced, and to remind the subject to complete the diary.

The study was considered completed after eight hours of evaluation or upon receipt of rescue medication. Subjects could discontinue the study at any time. Subjects who did not get adequate pain relief provided a final set of pain assessments and a global evaluation before taking rescue medication. Subjects were then given a rescue medication and pain assessments were discontinued. Subjects were encouraged to wait at least 90 minutes after administration of the study medication before using rescue medication. Subjects remedicating earlier than 90 minutes were not included in the analysis for efficacy.

For subjects who completed eight hours of evaluation without using rescue medication, the time of the first dose of analgesic within 24 hours after dosing with study medication was recorded on the take-home diary.

All subjects who received a dose of study medication returned to the study facility 5 to 9 days after surgery for a post treatment visit. The following was accomplished: (a) brief physical examination; (b) collection and review of subject's diary for 24-hour post-dosing adverse events, and medications (including rescue medications).

Efficacy evaluations were performed using primary and secondary efficacy (outcome) parameters. The primary efficacy parameters included:

(1) 8-hour Total Pain Relief Scores (TOTPAR-8) described below;

(2) 8-Hour Sum of Pain Intensity Difference Scores (SPID-8) described below;

(3) Time to Rescue;

(4) Percent of Subjects Remedicating with Rescue Medication; and

(5) Time to Onset of Meaningful Pain Relief.

The secondary efficacy parameters included:

(1) Hourly Pain Relief Scores;

(2) Hourly Pain Intensity Difference Scores;

(3) Maximum Pain Relief Scores;

(4) Peak Pain Intensity Difference Scores;

(5) Global Evaluations; and

(6) Time to Onset of First Perceptible Pain Relief.

Safety evaluations included (1) vital signs; and (2) adverse events. All adverse events were recorded on the case report forms (CRF) provided. Serious adverse events were reported promptly to the Institutional Review Board (IRB) and to the sponsor. The investigator transmitted a written report of the circumstances and outcome. All serious adverse events were reported to the FDA in compliance with Federal Regulations. An adverse event (AE) was defined as any untoward, noxious, or unintended event experienced by a subject in a clinical trial of an investigational agent, whether considered related to that investigational agent or not. A treatment-emergent adverse event was defined as an AE that was new in onset or aggravated in severity or frequency following administration of the investigational agent. A serious adverse event was defined as any AE occurring at any dose that resulted in any of the following outcomes: death, a life-threatening adverse drug experience, inpatient hospitalization or prolongation of existing hospitalization, a persistent or significant disability/incapacity, or congenital anomaly or birth defect.

A subject who completed Hour 8 or who completed at least 90 minutes and remedicated before Hour 8 was evaluable for efficacy. In any case, the reason for discontinuation was documented.

For the data analysis, parameters were computed as follows. The extent to which pain changes at each time point was measured by pain relief scores (PR, with 0=none, 1=a little, 2=some, 3=a lot, 4=complete), and pain intensity difference scores (PID, the difference between baseline and the current time, with the pain intensity scale consisting of 0=none, 1=mild, 2=moderate, 3=severe).

The extent to which pain changes over the entire test period was measured by the total pain relief score (TOTPAR-8), sum of pain intensity differences (SPID-8), maximum pain relief score (MAXPAR), peak pain intensity difference (PEAKPID), and global evaluation (0=poor, 1=fair, 2=good, 3=very good, 4=excellent). TOTPAR-8 and SPID-8 are defined as the sum of PR and PD, respectively, for the entire 8-hour observation period, weighted by the time difference between adjacent points (i.e., area under the curve using the trapezoidal rule). MAXPAR and PEAKPID are defined as the maximum of PR and PID, respectively.

Where required, the following imputation schemes were employed. Intermediate missing values were replaced by linear interpolation, whereas missing values after administration of rescue medication or other premature discontinuation were replaced by the last observation carried forward procedure (LOCF).

Further efficacy variables were time to rescue, percent of patients remedicating with rescue medication, time to onset of meaningful pain relief, and time to onset of first perceptible pain relief.

Safety was assessed through vital signs and adverse events (including body systems and preferred terms from the COSTART dictionary).

All testing of statistical significance were two-sided, and a difference resulting in a p-value of less than or equal to 0.05 was considered statistically significant.

Efficacy analyses was conducted on the intent-to-treat (ITT) analysis set, consisting of all randomized patients who received study medication. A second analysis could be done on the evaluable analysis set.

Demographic and baseline characteristics were summarized with descriptive statistics (for continuous variables) or frequencies (for categorical variables).

One-way analysis of variance (ANOVA) by treatment group was performed on PR, PD, TOTPAR-8, SPID-8, MAXPAR, PEAKPID, and the global evaluation (with PR and PID analyzed separately for each time point). Baseline pain intensity was investigated as a possible blocking factor, and baseline pain intensity VAS was investigated as a possible covariate. If the ANOVA treatment effect is significant at the p<0.05 level, one-sided Fisher's protected least significant difference test (LSD) was performed to investigate pairwise differences. For all pairwise comparisons, the error mean square from the overall analysis of variance with all treatments was used as the estimate of error variance.

Time to rescue (remedication) was analyzed using the Kaplan-Meier estimate to compute the survival distribution function. The distributions were compared among treatment groups using the log rank and Wilcoxon tests. A patient was considered censored at 24 hours if remedication had not occurred. Patients who dropped out because of reasons other than rescue medication were censored at the dropout time. The proportion of patients remedicating were compared among treatment groups using Fisher's exact test or a chi-squared test. Time to onset of meaningful pain relief and time to onset of first perceptible pain relief was analyzed in a similar fashion to time to rescue. Patients who did not achieve meaningful pain relief or perceptible pain relief were considered treatment failures and were assigned a time of 8 hours.

All patients who received study medication were assessed for clinical safety. Vital signs, including changes from baseline, were summarized with descriptive statistics. Adverse event frequencies were tabulated by body system and preferred term, and Fisher's exact test or a chi-squared test was used to test for differences in adverse event frequencies among the treatment groups by body system.

The sample size was estimated from historical data and from practical considerations rather than from calculation of expected measured differences.

A total of 204 subjects were randomized; among them 201 subjects were deemed evaluable. One subject in each of the placebo, MS and MS/0.1 NTX groups was not evaluable because the subject took rescue medication less than 90 minutes after dosing.

TABLE 1
Subject Disposition

| | | Treatments | | | | |
|---|---|---|---|---|---|---|
| | Placebo | MS (60 mg) | MS (60 mg) | MS (60 mg) | | |
| | with | with | with NTX | with NTX | MS (60 mg) with | |
| | Placebo | Placebo | (0.01 mg) | (0.1 mg) | NTX (1.0 mg) | Total |
| Number of Subjects Screened | 40 | 41 | 41 | 41 | 41 | 204 |
| Analyzed for Efficacy: | 40 | 41 | 41 | 41 | 41 | 204 |
| Intent-To-Treat | 39 | 40 | 41 | 40 | 41 | 201 |
| Evaluable Subjects | | | | | | |
| Analyzed for Safety: | 40 | 41 | 41 | 41 | 41 | 204 |
| Intent-To-Treat | | | | | | |

The demographic and baseline characteristics were summarized by treatment groups for the ITT population (all randomized patients) and the evaluable population (all randomized patients with at least one efficacy evaluation at 90 minutes or more after dosing) (Table 2). Demographic characteristics included age, race/ethnicity, sex, weight, height, medical history, teeth extracted (impacted and non-impacted), baseline pain intensity, and baseline visual analog scale.

The demographics for the ITT population were comparable across all 5 treatment groups. Subjects ranged in age from IS to 39 years; 67% were Caucasian and 51% were female. There was comparability among treatment groups regarding the degree of surgical trauma rating. For the evaluable population, but not for the ITT population, there was a difference among treatment groups in the maximum degree of impaction of third molar extracted. Patients in the placebo group had a lesser degree of bony impaction compared to patients in the low-dose group, and patients in both the low-dose and mid-dose groups had a greater degree of impaction compared to patients in the high-dose group. No adjustments in the analyses were made to take into account these differences among treatment groups. These differences had no influence on pain assessments at baseline. Generally, no differences among treatment groups were noted in the number of patients with either a significant medical history or disease of any body system. The baseline pain intensity scores and visual analog scale scores also were comparable across treatment groups (Table 3).

TABLE 2
Baseline Demographic Characteristics Intent-To-Treat Subjects
Treatments

| | | Placebo | | MS | | MS | | MS | | MS | | |
|---|---|---|---|---|---|---|---|---|---|---|---|---|
| Number of | | with | | (60 mg) with | | (60 mg) with | | (60 mg) with | | (60 mg) with | | |
| Subjects - 204 | | Placebo | | Placebo | | NTX (0.01 mg) | | NTX (0.1 mg) | | NTX 1.0 mg) | | P-Value |
| Sex (N, %) | Male | 18 | (45.0%) | 18 | (43.9%) | 21 | (51.2%) | 21 | (51.2%) | 21 | (51.2%) | 0.918 [2] |
| | Female | 22 | (55.0%) | 23 | (56.1%) | 20 | (48.8%) | 20 | (48.8%) | 20 | (48.8%) | |
| | Total | 40 | | 41 | | 41 | | 41 | | 41 | | |
| Age (yrs) | N | 40 | | 41 | | 41 | | 41 | | 41 | | 0.715 [1] |
| | Mean | 22.1 | | 22.8 | | 22.0 | | 23.1 | | 22.5 | | |
| | SD | 2.92 | | 3.87 | | 3.55 | | 5.10 | | 4.28 | | |
| | Median | 21.5 | | 22.0 | | 21.0 | | 22.0 | | 22.0 | | |
| | Range | 18-28 | | 19-32 | | 18-35 | | 16-39 | | 18-39 | | |
| Height (cm) | N | 40 | | 41 | | 41 | | 41 | | 41 | | 0.596 [1] |
| | Mean | 170.3 | | 170.7 | | 173.8 | | 171.4 | | 171.4 | | |
| | SD | 9.70 | | 12.22 | | 9.38 | | 10.87 | | 10.05 | | |
| | Median | 170.2 | | 167.6 | | 172.7 | | 172.7 | | 171.5 | | |
| | Range | 152.4-188.0 | | 149.9-198.1 | | 157.5-193.0 | | 139.7-194.3 | | 154.9-188.0 | | |
| Weight (kg) | N | 40 | | 41 | | 41 | | 41 | | 41 | | 0.384 [1] |
| | Mean | 68.8 | | 75.5 | | 72.1 | | 70.8 | | 72.6 | | |
| | SD | 13.94 | | 17.39 | | 12.99 | | 14.49 | | 17.34 | | |
| | Median | 67.3 | | 75.0 | | 73.2 | | 70.9 | | 69.8 | | |
| | Range | 47.3-106.4 | | 42.7-117.3 | | 50.9-105.5 | | 46.4-104.5 | | 47.3-122.3 | | |
| Ethnic Origin | Caucasian | 26 | (65.0%) | 25 | (61.0%) | 31 | (75.6%) | 28 | (68.3%) | 26 | (63.4%) | 0.666 [2] |
| | Black | 4 | (10.0%) | 4 | (9.8%) | 1 | (2.4%) | 1 | (2.4%) | 3 | (7.3%) | |
| | Hispanic | 7 | (17.5%) | 11 | (26.8%) | 7 | (17.1%) | 9 | (22.0%) | 6 | (14.6%) | |
| | Asian | 3 | (7.5%) | 1 | (2.4%) | 1 | (2.4%) | 2 | (4.9%) | 5 | (12.2%) | |
| | Other | 0 | (0.0%) | 0 | (0.0%) | 1 | (2.4%) | 1 | (2.4%) | 1 | (2.4%) | |
| | Total | 40 | | 41 | | 41 | | 41 | | 41 | | |

[1] ONE-WAY ANALYSIS OF VARIANCE WITH TREATMENT AS THE FACTOR
[2] FISHER'S EXACT TEST.
[3] BLACK, ASIAN, HISPANIC, AND OTHER ARE COMBINED INTO ONE CATEGORY TO DERIVE P-VALUE.

TABLE 3
Summary of Baseline Pain Intensity Scores
Intent-To-Treat Population

| | | | | P-VALUE FOR PAIRWISE COMPARISONS | | | |
|---|---|---|---|---|---|---|---|
| | | | | | | | P-VALUE FOR |
| PAIN INTENSITY | | | | MS 60 mg | MS 60 mg | MS 60 mg | OVERALL |
| TREATMENT | MODERATE | SEVERE | MS 60 mg | NTX 0.01 mg | NTX 0.1 mg | NTX 1 mg | TREATMENT |
| Placebo | 16 (40.0%) | 24 (60.0%) | 0.822 | 1.000 | 0.822 | 1.000 | 0.997 |
| MS 60 mg | 18 (43.9%) | 23 (56.1%) | | 1.000 | 1.000 | 1.000 | |
| MS 60 mg/NTX 0.01 mg | 17 (41.5%) | 24 (58.5%) | | | 1.000 | 1.000 | |
| MS 60 mg/NTX 0.1 mg | 18 (43.9%) | 23 (56.1%) | | | | 1.000 | |
| MS 60 mg/NTX 1 mg | 17 (41.5%) | 24 (58.5%) | | | | | |

NOTE: P-VALUES ARE FROM FISHER'S EXACT TEST.

Summary of Baseline Visual Analog Scale (VAS) Scores
Intent-To-Treat Population

| | | | | | | | | | | P-VALUE FOR PAIRWISE COMPARISONS | | | | |
|---|---|---|---|---|---|---|---|---|---|---|---|---|---|---|
| | | | | | | | | | | | | | | P-Value |
| BASELINE VAS SCORE | | | | | | | | | | | | | | for |
| | Moderate [1] | | | Severe [1] | | | Total | | | | MS 60 mg | MS 60 mg | MS 60 mg | Overall |
| TREATMENT | N | Mean | (SD) | N | Mean | (SD) | N | Mean | (SD) | MS 60 mg | NTX 0.01 mg | NTX 0.1 mg | NTX 1 mg | Treatment |
| Placebo | 16 | 65.5 | (7.91) | 24 | 79.4 | (9.91) | 40 | 73.9 | (11.39) | 0.250 | 0.890 | 0.296 | 0.966 | 0.512 |
| MS 60 mg | 18 | 68.1 | (6.58) | 23 | 84.1 | (8.23) | 41 | 77.1 | (11.00) | | 0.195 | 0.922 | 0.231 | |
| MS 60 mg/NTX | 17 | 60.7 | (9.29) | 24 | 82.5 | (10.77) | 41 | 73.5 | (14.81) | | | 0.234 | 0.923 | |
| 0.01 mg | | | | | | | | | | | | | | |
| MS 60 mg/NTX | 17 | 65.5 | (10.62) | 23 | 85.2 | (9.18) | 40 | 76.8 | (13.83) | | | | 0.274 | |
| 0.1 mg | | | | | | | | | | | | | | |
| MS 60 mg/NTX | 17 | 67.6 | (10.53) | 24 | 78.1 | (10.23) | 41 | 73.7 | (11.48) | | | | | |
| 1 mg | | | | | | | | | | | | | | |

NOTE:
P-VALUES ARE FROM ONE-WAY ANALYSIS OF VARIANCE AND ITS CONTRASTS.
[1] BASELINE PAIN INTENSITY ON THE CATEGORICAL SCALE.

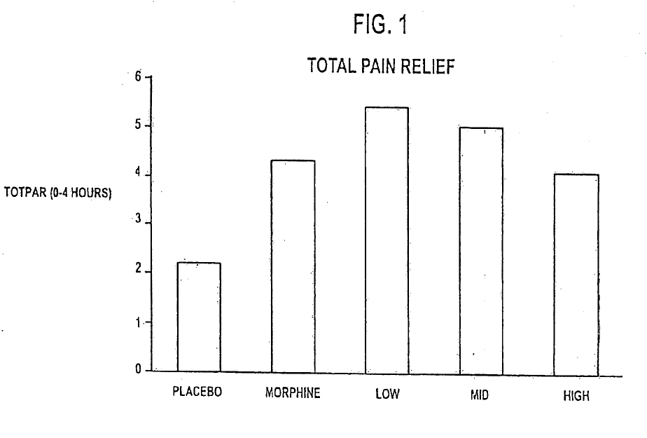

The TOTPAR results (4-hour, 6-hour, 8-hour) are summarized in Table 4 and the 4-hour TOTPAR scores are shown in FIG. 1. The placebo treatment group had the lowest mean TOTPAR scores. All 4 of the active treatment groups exhibited mean TOTPAR scores that were numerically higher than placebo. The combination treatments had a reverse dose-response relation in the mean TOTPAR scores, i.e., the highest dose of NTX had the lowest mean TOTPAR scores and the lowest dose of NTX had the highest mean TOTPAR scores. This pattern (low-dose (0.01 mg NTX)>mid-dose (1.0 mg NTX) was observed for all pain relief variables throughout the study. The mean TOTPAR scores for the 0.01-mg NTX and 0.1-mg NTX combination treatments were higher than that for the MS alone treatment, whereas the 1.0-mg NTX combination treatment mean was comparable to or lower than that for the MS alone treatment (FIG. 1).

Analyses of TOTPAR for the evaluable subgroup yielded results similar to those for the ITT population.

TABLE 4
Total Pain Relief Scores
Intent-To-Treat Population

| TOTAL PAIN RELIEF SCORE | | | | | | | | P-VALUE | P-VALUE |
|---|---|---|---|---|---|---|---|---|---|
| TREATMENT | N | MEAN | SD | MIN | MEDIAN | MAX | SOURCE | [1] | [2] |

TOTAL PAIN RELIEF SCORE (0-4 HOURS)

| | | | | | | | | | |
|---|---|---|---|---|---|---|---|---|---|
| A) Placebo | 40 | 2.20 | 2.836 | 0.0 | 0.25 | 9.5 | TRT | 0.003** | 0.004** |
| B) MS 60 mg | 41 | 4.38 | 4.035 | 0.0 | 3.75 | 13.2 | BASEPI | N/A | 0.312 |
| C) MS 60 mg/NTX 0.01 mg | 41 | 5.50 | 4.106 | 0.0 | 5.73 | 14.0 | BASEPI * TRT | N/A | 0.081 |
| D) MS 60 mg/NTX 0.1 mg | 41 | 5.09 | 4.278 | 0.0 | 3.25 | 12.3 | B-A | 0.014* | 0.013* |
| E) MS 60 mg/NTX 1 mg | 41 | 4.18 | 4.439 | 0.0 | 2.75 | 14.0 | C-A | <0.001*** | <0.001*** |
| | | | | | | | D-A | 0.001** | 0.001** |
| | | | | | | | E-A | 0.026* | 0.024* |
| | | | | | | | C-B | 0.203 | 0.198 |
| | | | | | | | D-B | 0.416 | 0.411 |
| | | | | | | | E-B | 0.828 | 0.826 |

TOTAL PAIN RELIEF SCORE (0-6 HOURS)

| | | | | | | | | | |
|---|---|---|---|---|---|---|---|---|---|
| A) Placebo | 40 | 3.62 | 4.851 | 0.0 | 0.25 | 14.5 | TRT | 0.004** | 0.006** |
| B) MS 60 mg | 41 | 7.52 | 6.962 | 0.0 | 8.25 | 21.2 | BASEPI | N/A | 0.419 |
| C) MS 60 mg/NTX 0.01 mg | 41 | 8.85 | 6.470 | 0.0 | 9.23 | 20.5 | BASEPI * TRT | N/A | 0.044* |
| D) MS 60 mg/NTX 0.1 mg | 41 | 8.25 | 7.089 | 0.0 | 6.75 | 20.3 | B-A | 0.008** | 0.007** |
| E) MS 60 mg/NTX 1 mg | 41 | 6.60 | 7.277 | 0.0 | 2.75 | 22.0 | C-A | <0.001*** | <0.001*** |
| | | | | | | | D-A | 0.001** | 0.001** |
| | | | | | | | E-A | 0.043* | 0.041* |
| | | | | | | | C-B | 0.359 | 0.353 |
| | | | | | | | D-B | 0.613 | 0.608 |
| | | | | | | | E-B | 0.530 | 0.524 |

TOTAL PAIN RELIEF SCORE (0-8 HOURS)

| | | | | | | | | | |
|---|---|---|---|---|---|---|---|---|---|
| A) Placebo | 40 | 5.12 | 7.026 | 0.0 | 0.25 | 20.5 | TRT | 0.007** | 0.009** |
| B) MS 60 mg | 41 | 10.73 | 9.988 | 0.0 | 13.50 | 29.2 | BASEPI | N/A | 0.470 |
| C) MS 60 mg/NTX 0.01 mg | 41 | 12.15 | 9.139 | 0.0 | 11.75 | 27.5 | BASEPI * TRT | N/A | 0.037* |
| D) MS 60 mg/NTX 0.1 mg | 41 | 11.52 | 10.130 | 0.0 | 10.75 | 28.3 | B-A | 0.007** | 0.007** |
| E) MS 60 mg/NTX 1 mg | 41 | 9.14 | 10.337 | 0.0 | 2.75 | 30.0 | C-A | <0.001*** | <0.001*** |
| | | | | | | | D-A | 0.002** | 0.002** |
| | | | | | | | E-A | 0.056 | 0.053 |
| | | | | | | | C-B | 0.496 | 0.489 |
| | | | | | | | D-B | 0.705 | 0.701 |
| | | | | | | | E-B | 0.442 | 0.436 |

[1] FROM ONE-WAY ANALYSIS OF VARIANCE AND FISHER'S PROTECTED LEAST SIGNIFICANT DIFFERENCE TEST.
[2] FROM TWO-WAY ANALYSIS OF VARIANCE WITH BASELINE PAIN INTENSITY AS A BLOCKING FACTOR AND FISHER'S PROTECTED LEAST SIGNIFICANT DIFFERENCE TEST.
*, **, ***P-VALUE <=0.05, <=0.01, or <=0.001 RESPECTIVELY.
N/A: NOT APPLICABLE

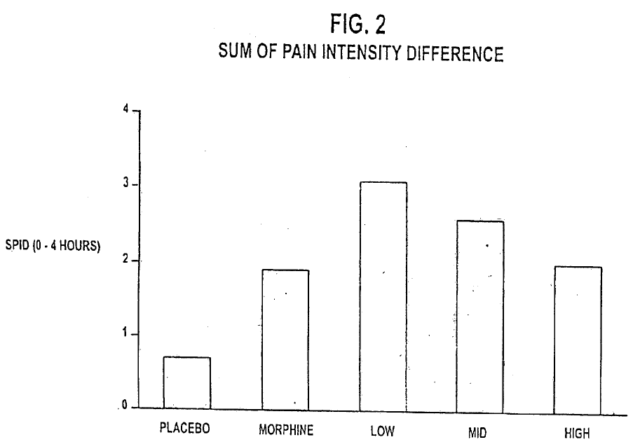

Table 5 summarizes the results of the 4, 6, and 8-hour SPID results. The 4-hour results are also represented in FIG. 2. The placebo treatment had the lowest mean 4-hour SPID scores (0.68±2.165). All 4 of the active treatment groups exhibited improved profiles in mean SPID relative to placebo. The mean SPID scores for the 0.01-mg NTX and 0.1-mg NTX combination treatments were higher than that for the MS alone treatment, whereas the 1.0-mg NTX combination treatment was comparable to that for the MS alone treatment (FIG. 2).

The patterns of the 6-hour and 8-hour SPID scores were similar to those at 4 hours. Analyses of SPID for the evaluable subgroup also yielded profiles that were similar to those found in the ITT population.

TABLE 5
Summary of Pain Intensity Differences
Intent-To-Treat Population

| SUM OF PAIN INTENSITY DIFFERENCES [1] | | | | | | | | P-VALUE | P-VALUE |
|---|---|---|---|---|---|---|---|---|---|
| TREATMENT | N | MEAN | SD | MIN | MEDIAN | MAX | SOUCRE | [2] | [3] |

SUMMARY OF PAIN INTENSITY DIFFERENCES (0-4 HOURS)

| | | | | | | | | | |
|---|---|---|---|---|---|---|---|---|---|
| A) Placebo | 40 | 0.68 | 2.165 | −3.8 | 0.00 | 5.0 | TRT | 0.009** | 0.003** |
| B) MS 60 mg | 41 | 1.91 | 3.296 | −3.8 | 2.50 | 8.0 | BASEPI | N/A | <0.001*** |
| C) MS 60 mg/NTX 0.01 mg | 41 | 3.08 | 3.309 | −3.8 | 3.24 | 10.3 | BASEPI * TRT | N/A | 0.040* |
| D) MS 60 mg/NTX 0.1 mg | 41 | 2.62 | 2.790 | −3.8 | 2.48 | 8.5 | B-A | 0.077 | 0.048* |
| E) MS 60 mg/NTX 1 mg | 41 | 2.01 | 3.763 | −3.8 | 1.25 | 8.5 | C-A | <0.001*** | <0.001*** |
| | | | | | | | D-A | 0.005** | 0.001** |
| | | | | | | | E-A | 0.054* | 0.031* |
| | | | | | | | C-B | 0.090 | 0.058 |
| | | | | | | | D-B | 0.302 | 0.248 |
| | | | | | | | E-B | 0.875 | 0.860 |

SUMMARY OF PAIN INTENSITY DIFFERENCES (0-6 HOURS)

| | | | | | | | | | |
|---|---|---|---|---|---|---|---|---|---|
| A) Placebo | 40 | 1.15 | 3.435 | −5.8 | 0.00 | 8.3 | TRT | 0.013* | 0.004** |
| B) MS 60 mg | 41 | 3.33 | 5.510 | −5.8 | 4.50 | 12.0 | BASEPI | N/A | <0.001*** |
| C) MS 60 mg/NTX 0.01 mg | 41 | 4.86 | 5.069 | −5.8 | 5.25 | 15.3 | BASEPI * TRT | N/A | 0.021* |
| D) MS 60 mg/NTX 0.1 mg | 41 | 4.36 | 4.606 | −5.8 | 4.48 | 14.5 | B-A | 0.053 | 0.031* |
| E) MS 60 mg/NTX 1 mg | 41 | 3.20 | 6.136 | −5.8 | 1.25 | 14.5 | C-A | 0.001** | <0.001*** |
| | | | | | | | D-A | 0.004** | 0.001** |
| | | | | | | | E-A | 0.068 | 0.042* |
| | | | | | | | C-B | 0.170 | 0.127 |
| | | | | | | | D-B | 0.355 | 0.303 |
| | | | | | | | E-B | 0.911 | 0.901 |

SUMMARY OF PAIN INTENSITY DIFFERENCES (0-8 HOURS)

| | | | | | | | | | |
|---|---|---|---|---|---|---|---|---|---|
| A) Placebo | 40 | 1.65 | 4.781 | −7.8 | 0.00 | 12.8 | TRT | 0.019* | 0.007** |
| B) MS 60 mg | 41 | 4.80 | 7.821 | −7.8 | 6.50 | 17.3 | BASEPI | N/A | <0.001*** |
| C) MS 60 mg/NTX 0.01 mg | 41 | 6.62 | 7.090 | −7.8 | 7.25 | 19.8 | BASEPI * TRT | N/A | 0.016* |
| D) MS 60 mg/NTX 0.1 mg | 41 | 6.18 | 6.581 | −7.8 | 6.49 | 20.5 | B-A | 0.048* | 0.028* |
| E) MS 60 mg/NTX 1 mg | 41 | 4.54 | 8.716 | −7.8 | 1.25 | 20.0 | C-A | 0.001** | <0.001*** |
| | | | | | | | D-A | 0.004** | 0.001** |
| | | | | | | | E-A | 0.069 | 0.043* |
| | | | | | | | C-B | 0.248 | 0.199 |
| | | | | | | | D-B | 0.380 | 0.329 |
| | | | | | | | E-B | 0.870 | 0.855 |

[1] PAIN INTENSITY DIFFERENCE = PAIN INTENSITY AT BASELINE − PAIN INTENSITY AT CURRENT TIME.
[2] FROM ONE-WAY ANALYSIS OF VARIANCE AND FISHER'S PROTECTED LEAST SIGNIFICANT DIFFERENCE TEST.
[3] FROM TWO-WAY ANALYSIS OF VARIANCE WITH BASELINE PAIN INTENSITY AS A BLOCKING FACTOR AND FISHER'S PROTECTED LEAST SIGNIFICANT DIFFERENCE TEST.
*, **, ***P-VALUE <=0.05, <=0.01, or <=0.001 RESPECTIVELY.
N/A: NOT APPLICABLE

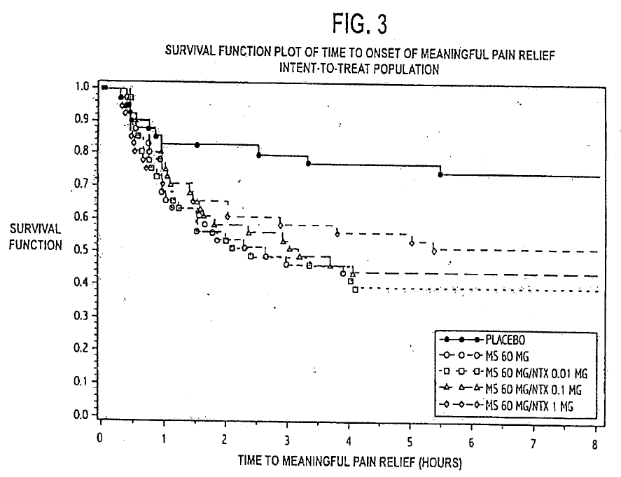

FIG. 3 is a visual presentation of the summary and analysis of time to onset of meaningful pain relief scores presented in Table 6. The median time to onset of meaningful pain relief was shortest in the 0.01-mg NTX (low-dose) combination treatment group. The placebo treatment had the lower number of subjects who reached meaningful pain relief.

Analyses of times to onset of meaningful pain relief for the evaluable subgroup yielded similar result.

TABLE 6
Time To Onset of Meaningful Pain Relief
Intent-To-Treat Population

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 40 | >8:00 | (>8:00, >8:00) | TREATMENT | 0.029* | 0.062 |
| B) MS 60 mg | 41 | 2:37 | (1:07, >8:00) | B-A | 0.006** | N/D |
| C) MS 60 mg/NTX 0.01 mg | 41 | 2:23 | (1:12, >8:00) | C-A | 0.001** | N/D |
| D) MS 60 mg/NTX 0.1 mg | 41 | 3:10 | (1:33, >8:00) | D-A | 0.007** | N/D |
| E) MS 60 mg/NTX 1 mg | 41 | >8:00 | (2:00, >8:00) | E-A | 0.030* | N/D |
| | | | | C-B | 0.725 | N/D |
| | | | | D-B | 0.830 | N/D |
| | | | | E-B | 0.592 | N/D |

*, **, ***P-VALUE <=0.05, <=0.01, or <=0.001 RESPECTIVELY.
N/D: NOT DONE (BECAUSE OVERALL P-VALUE NOT SIGNIFICANT).

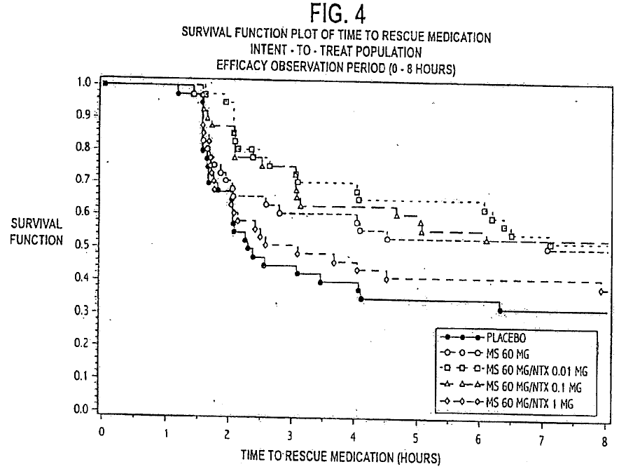

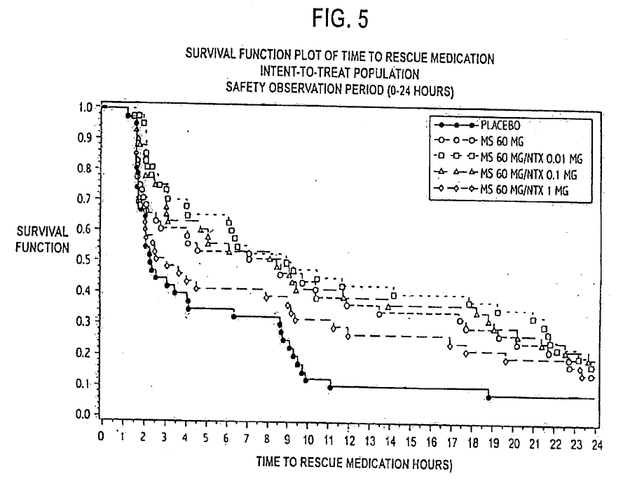

FIGS. 4 and 5 are a visual presentation of the summary and analysis of time to remedication (rescue medication) up to 8 and 24 hours presented in Table 7. The survival distributions (0-8 hours) were different across treatment groups. The survival distributions were different for the low-dose and mid-dose groups compared to placebo (FIG. 4). The median times to administration of rescue medication were longer for the morphine (>8 hours), low-dose (>8 hours), and mid-dose (>8 hours) groups compared to the high-dose (3 hours, 4 minutes) and placebo (2 hours, 18 minutes) groups.

The survival distributions (0-24 hours) were also different across treatment groups, and were also different for the morphine, low-dose, and mid-dose groups compared to the placebo group (FIG. 5). Again, the median times to administration of rescue medication were longer for the morphine, low-dose, and mid-dose groups.

Analyses of time to remedication up to 24 hours yielded similar results, however, the data should be viewed with caution because subjects were not under close supervision after 8 hours. Analyses for the evaluable subjects yielded results similar to those for the ITT population.

TABLE 7
Time To Rescue Medication
Intent-To-Treat Population

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |

EFFICACY OBSERVATION PERIOD (0-8 HOURS)

| | | | | | | |
|---|---|---|---|---|---|---|
| A) Placebo | 40 | 2:18 | (2:02, 4:05) | TREATMENT | 0.047* | 0.014* |
| B) MS 60 mg | 41 | >8:00 | (2:33, >8:00) | B-A | 0.092 | 0.114 |
| C) MS 60 mg/NTX 0.01 mg | 41 | >8:00 | (6:03, >8:00) | C-A | 0.011* | 0.002** |
| D) MS 60 mg/NTX 0.1 mg | 41 | >8:00 | (3:06, >8:00) | D-A | 0.020* | 0.010* |
| E) MS 60 mg/NTX 1 mg | 41 | 3:04 | (2:00, >8:00) | E-A | 0.506 | 0.471 |
| | | | | C-B | 0.506 | 0.234 |
| | | | | D-B | 0.605 | 0.422 |
| | | | | E-B | 0.285 | 0.347 |

SAFETY OBSERVATION PERIOD (0-24 HOURS)

| | | | | | | |
|---|---|---|---|---|---|---|
| A) Placebo | 40 | 2:18 | (2:02, 4:05) | TREATMENT | 0.015* | 0.003* |
| B) MS 60 mg | 41 | 8:37 | (2:33, 13:28) | B-A | 0.029* | 0.043* |
| C) MS 60 mg/NTX 0.01 mg | 41 | 9:14 | (6:03, 20:59) | C-A | 0.001** | <0.001*** |
| D) MS 60 mg/NTX 0.1 mg | 41 | 8:26 | (3:06, 18:17) | D-A | 0.005** | 0.003** |
| E) MS 60 mg/NTX 1 mg | 41 | 3:04 | (2:00, 9:09) | E-A | 0.169 | 0.266 |
| | | | | C-B | 0.388 | 0.167 |
| | | | | D-B | 0.539 | 0.424 |
| | | | | E-B | 0.562 | 0.427 |

*, **, ***P-VALUE <=0.05, <=0.01, or <=0.001 RESPECTIVELY.

Table 8 presents the summary and analysis of percent of subjects who took remedication up to 5 and 24 hours. Analyses of the percentage of subjects who remedicated within 24 hours indicated that all 5 treatment groups were comparable, however, the data should be interpreted with caution because subjects were not under close supervision after 8 hours. Analyses for the evaluable subjects led to conclusions similar to those for the ITT population.

TABLE 8
Percent of Subjects Rescued
Intent-To-Treat Population
RESCUED

| | | | | |
|---|---|---|---|---|
| TREATMENT | YES | NO | SOURCE | P-VALUE [1] |

EFFICACY OBSERVATION PERIOD (0-8 HOURS)

| | | | | |
|---|---|---|---|---|
| A) Placebo | 27 (67.5%) | 13 (32.5%) | TREATMENT | 0.193 |
| B) MS 60 mg | 20 (48.8%) | 21 (51.2%) | B-A | N/D |
| C) MS 60 mg/NTX 0.01 mg | 19 (46.3%) | 22 (53.7%) | C-A | N/D |
| D) MS 60 mg/NTX 0.1 mg | 19 (46.3%) | 22 (53.7%) | D-A | N/D |
| E) MS 60 mg/NTX 1 mg | 25 (61.0%) | 16 (39.0%) | E-A | N/D |
| | | | C-B | N/D |
| | | | D-B | N/D |
| | | | E-B | N/D |

SAFETY OBSERVATION PERIOD (0-24 HOURS)

| | | | | |
|---|---|---|---|---|
| A) Placebo | 37 (92.5%) | 3 (7.5%) | TREATMENT | 0.536 |
| B) MS 60 mg | 35 (85.4%) | 6 (14.6%) | B-A | N/D |
| C) MS 60 mg/NTX 0.01 mg | 33 (80.5%) | 8 (19.5%) | C-A | N/D |
| D) MS 60 mg/NTX 0.1 mg | 33 (80.5%) | 8 (19.5%) | D-A | N/D |
| E) MS 60 mg/NTX 1 mg | 35 (85.4%) | 6 (14.6%) | E-A | N/D |
| | | | C-B | N/D |
| | | | D-B | N/D |
| | | | E-B | N/D |

N/D: NOT DONE (BECAUSE OVERALL P-VALUE NOT SIGNIFICANT).

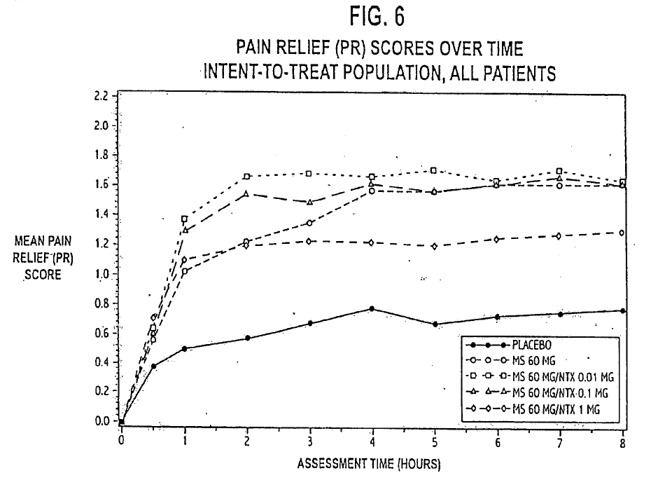

FIG. 6 is a visual presentation of the hourly pain relief scores presented in Table 9. The hourly pain relief scores were summarized and analyzed in 2 ways: first as a categorical variable and second as a numerical variable. Because results of these two methods were similar, only the results from the numerical version are presented here. Whereas the hourly pain relief scores for the placebo treatment were less than those for the active treatment groups which improved over time. There was separation between the placebo and the active treatment groups that continued throughout the S-hour study period. Comparable pain relief was observed (see, e.g., 1-3 hours) in the MS alone group and the high-dose (1.0 mg NTX) combination group (FIG. 6). Highest pain relief scores were observed for the low-dose (0.01 mg NTX) combination group (FIG. 6).

TABLE 9
Pain Relief (PR) Scores [1]
Intent-To-Treat Population

| PAIN RELIEF SCORE (PR) | | | | | | | | | |
|---|---|---|---|---|---|---|---|---|---|
| TREATMENT | N | MEAN | SD | MIN | MEDIAN | MAX | SOURCE | P-VALUE [2] | P-VALUE [3] |

30 MINUTES

| | | | | | | | | | |
|---|---|---|---|---|---|---|---|---|---|
| A) Placebo | 40 | 0.38 | 0.628 | 0 | 0.00 | 2 | TRT | 0.522 | 0.552 |
| B) MS 60 mg | 41 | 0.56 | 0.923 | 0 | 0.00 | 4 | BASEPI | N/A | 0.535 |
| C) MS 60 mg/NTX 0.01 mg | 41 | 0.63 | 0.888 | 0 | 0.00 | 3 | BASEPI * TRT | N/A | 0.959 |
| D) MS 60 mg/NTX 0.1 mg | 41 | 0.61 | 0.997 | 0 | 0.00 | 3 | B-A | N/D | N/D |
| E) MS 60 mg/NTX 1 mg | 41 | 0.71 | 0.929 | 0 | 0.00 | 3 | C-A | N/D | N/D |
| | | | | | | | D-A | N/D | N/D |
| | | | | | | | E-A | N/D | N/D |
| | | | | | | | C-B | N/D | N/D |
| | | | | | | | D-B | N/D | N/D |
| | | | | | | | E-B | N/D | N/D |

1 HOUR

| | | | | | | | | | |
|---|---|---|---|---|---|---|---|---|---|
| A) Placebo | 40 | 0.50 | 0.934 | 0 | 0.00 | 4 | TRT | 0.004** | 0.009** |
| B) MS 60 mg | 41 | 1.02 | 0.908 | 0 | 1.00 | 3 | BASEPI | N/A | 0.337 |
| C) MS 60 mg/NTX 0.01 mg | 41 | 1.37 | 1.280 | 0 | 1.00 | 4 | BASEPI * TRT | N/A | 0.627 |
| D) MS 60 mg/NTX 0.1 mg | 41 | 1.29 | 1.167 | 0 | 1.00 | 4 | B-A | 0.032* | 0.033* |
| E) MS 60 mg/NTX 1 mg | 41 | 1.10 | 1.114 | 0 | 1.00 | 4 | C-A | <0.001*** | <0.001*** |
| | | | | | | | D-A | 0.001** | 0.001** |
| | | | | | | | E-A | 0.014* | 0.014* |
| | | | | | | | C-B | 0.153 | 0.154 |
| | | | | | | | D-B | 0.260 | 0.261 |
| | | | | | | | E-B | 0.749 | 0.750 |

2 HOURS

| | | | | | | | | | |
|---|---|---|---|---|---|---|---|---|---|
| A) Placebo | 40 | 0.58 | 0.813 | 0 | 0.00 | 3 | TRT | <0.001*** | <0.001*** |
| B) MS 60 mg | 41 | 1.22 | 1.235 | 0 | 1.00 | 4 | BASEPI | N/A | 0.169 |
| C) MS 60 mg/NTX 0.01 mg | 41 | 1.66 | 1.237 | 0 | 2.00 | 4 | BASEPI * TRT | N/A | 0.054 |
| D) MS 60 mg/NTX 0.1 mg | 41 | 1.54 | 1.267 | 0 | 1.00 | 4 | B-A | 0.015* | 0.013* |
| E) MS 60 mg/NTX 1 mg | 41 | 1.20 | 1.289 | 0 | 1.00 | 4 | C-A | <0.001*** | <0.001*** |
| | | | | | | | D-A | <0.001*** | <0.001*** |
| | | | | | | | E-A | 0.019* | 0.017* |
| | | | | | | | C-B | 0.094 | 0.089 |
| | | | | | | | D-B | 0.226 | 0.219 |
| | | | | | | | E-B | 0.925 | 0.924 |

3 HOURS

| | | | | | | | | | |
|---|---|---|---|---|---|---|---|---|---|
| A) Placebo | 40 | 0.68 | 0.997 | 0 | 0.00 | 3 | TRT | 0.010* | 0.013* |
| B) MS 60 mg | 41 | 1.34 | 1.334 | 0 | 1.00 | 4 | BASEPI | N/A | 0.515 |
| C) MS 60 mg/NTX 0.01 mg | 41 | 1.68 | 1.404 | 0 | 1.00 | 4 | BASEPI * TRT | N/A | 0.032* |
| D) MS 60 mg/NTX 0.1 mg | 41 | 1.49 | 1.362 | 0 | 1.00 | 4 | B-A | 0.023* | 0.021* |
| E) MS 60 mg/NTX 1 mg | 41 | 1.22 | 1.423 | 0 | 0.00 | 4 | C-A | <0.001*** | <0.001*** |
| | | | | | | | D-A | 0.005** | 0.005** |
| | | | | | | | E-A | 0.063 | 0.060 |
| | | | | | | | C-B | 0.241 | 0.234 |
| | | | | | | | D-B | 0.614 | 0.609 |
| | | | | | | | E-B | 0.675 | 0.670 |

4 HOURS

| | | | | | | | | | |
|---|---|---|---|---|---|---|---|---|---|
| A) Placebo | 40 | 0.78 | 1.187 | 0 | 0.00 | 4 | TRT | 0.027* | 0.030* |
| B) MS 60 mg | 41 | 1.56 | 1.501 | 0 | 2.00 | 4 | BASEPI | N/A | 0.460 |
| C) MS 60 mg/NTX 0.01 mg | 41 | 1.66 | 1.353 | 0 | 2.00 | 4 | BASEPI * TRT | N/A | 0.018* |
| D) MS 60 mg/NTX 0.1 mg | 41 | 1.61 | 1.498 | 0 | 1.00 | 4 | B-A | 0.013* | 0.011* |
| E) MS 60 mg/NTX 1 mg | 41 | 1.22 | 1.492 | 0 | 0.00 | 4 | C-A | 0.005** | 0.004** |
| | | | | | | | D-A | 0.008** | 0.007** |
| | | | | | | | E-A | 0.158 | 0.150 |
| | | | | | | | C-B | 0.754 | 0.750 |
| | | | | | | | D-B | 0.875 | 0.873 |
| | | | | | | | E-B | 0.275 | 0.266 |

5 HOURS

| | | | | | | | | | |
|---|---|---|---|---|---|---|---|---|---|
| A) Placebo | 40 | 0.68 | 0.997 | 0 | 0.00 | 3 | TRT | 0.008** | 0.009** |
| B) MS 60 mg | 41 | 1.56 | 1.534 | 0 | 2.00 | 4 | BASEPI | N/A | 0.818 |
| C) MS 60 mg/NTX 0.01 mg | 41 | 1.71 | 1.453 | 0 | 2.00 | 4 | BASEPI * TRT | N/A | 0.045* |
| D) MS 60 mg/NTX 0.1 mg | 41 | 1.56 | 1.534 | 0 | 1.00 | 4 | B-A | 0.005** | 0.004** |
| E) MS 60 mg/NTX 1 mg | 41 | 1.20 | 1.487 | 0 | 0.00 | 4 | C-A | 0.001** | 0.001** |
| | | | | | | | D-A | 0.005** | 0.004** |
| | | | | | | | E-A | 0.100 | 0.096 |
| | | | | | | | C-B | 0.640 | 0.636 |
| | | | | | | | D-B | 1.000 | 1.000 |
| | | | | | | | E-B | 0.243 | 0.238 |

6 HOURS

| | | | | | | | | | |
|---|---|---|---|---|---|---|---|---|---|
| A) Placebo | 40 | 0.73 | 1.086 | 0 | 0.00 | 3 | TRT | 0.024* | 0.029* |
| B) MS 60 mg | 41 | 1.61 | 1.547 | 0 | 2.00 | 4 | BASEPI | N/A | 0.534 |
| C) MS 60 mg/NTX 0.01 mg | 41 | 1.63 | 1.479 | 0 | 1.00 | 4 | BASEPI * TRT | N/A | 0.026* |
| D) MS 60 mg/NTX 0.1 mg | 41 | 1.61 | 1.611 | 0 | 1.00 | 4 | B-A | 0.007** | 0.006** |
| E) MS 60 mg/NTX 1 mg | 41 | 1.24 | 1.562 | 0 | 0.00 | 4 | C-A | 0.005** | 0.005** |
| | | | | | | | D-A | 0.007** | 0.006** |
| | | | | | | | E-A | 0.114 | 0.108 |
| | | | | | | | C-B | 0.940 | 0.939 |
| | | | | | | | D-B | 1.000 | 1.000 |
| | | | | | | | E-B | 0.261 | 0.253 |

7 HOURS

| | | | | | | | | | |
|---|---|---|---|---|---|---|---|---|---|
| A) Placebo | 40 | 0.75 | 1.127 | 0 | 0.00 | 3 | TRT | 0.026* | 0.029* |
| B) MS 60 mg | 41 | 1.61 | 1.595 | 0 | 1.00 | 4 | BASEPI | N/A | 0.616 |
| C) MS 60 mg/NTX 0.01 mg | 41 | 1.71 | 1.569 | 0 | 1.00 | 4 | BASEPI * TRT | N/A | 0.036* |
| D) MS 60 mg/NTX 0.1 mg | 41 | 1.66 | 1.622 | 0 | 1.00 | 4 | B-A | 0.011* | 0.010* |
| E) MS 60 mg/NTX 1 mg | 41 | 1.27 | 1.613 | 0 | 0.00 | 4 | C-A | 0.005** | 0.004** |
| | | | | | | | D-A | 0.007** | 0.006** |
| | | | | | | | E-A | 0.126 | 0.120 |
| | | | | | | | C-B | 0.771 | 0.768 |
| | | | | | | | D-B | 0.884 | 0.882 |
| | | | | | | | E-B | 0.309 | 0.303 |

8 HOURS

| | | | | | | | | | |
|---|---|---|---|---|---|---|---|---|---|
| A) Placebo | 40 | 0.78 | 1.187 | 0 | 0.00 | 4 | TRT | 0.056 | 0.067 |
| B) MS 60 mg | 41 | 1.61 | 1.595 | 0 | 1.00 | 4 | BASEPI | N/A | 0.709 |
| C) MS 60 mg/NTX 0.01 mg | 41 | 1.63 | 1.577 | 0 | 1.00 | 4 | BASEPI * TRT | N/A | 0.088 |
| D) MS 60 mg/NTX 0.1 mg | 41 | 1.61 | 1.611 | 0 | 1.00 | 4 | B-A | N/D | N/D |
| E) MS 60 mg/NTX 1 mg | 41 | 1.29 | 1.632 | 0 | 0.00 | 4 | C-A | N/D | N/D |
| | | | | | | | D-A | N/D | N/D |
| | | | | | | | E-A | N/D | N/D |
| | | | | | | | C-B | N/D | N/D |
| | | | | | | | D-B | N/D | N/D |
| | | | | | | | E-B | N/D | N/D |

[1] PAIN RELIEF (PR) SCORES: 0 = NONE, 1 = A LITTLE, 2 = SOME, 3 = A LOT, 4 = COMPLETE.
[2] FROM ONE-WAY ANALYSIS OF VARIANCE AND FISHER'S PROTECTED LEAST SIGNIFICANT DIFFERENCE TEST.
[3] FROM TWO-WAY ANALYSIS OF VARIANCE WITH BASELINE PAIN INTENSITY AS A BLOCKING FACTOR AND FISHER'S PROTECTED LEAST SIGNIFICANT DIFFERENCE TEST.
*, **, ***P-VALUE <=0.05, <=0.01, or <=0.001 RESPECTIVELY.
N/A: NOT APPLICABLE, N/D: NOT DONE (BECAUSE OVERALL P-VALUE NOT SIGNIFICANT).

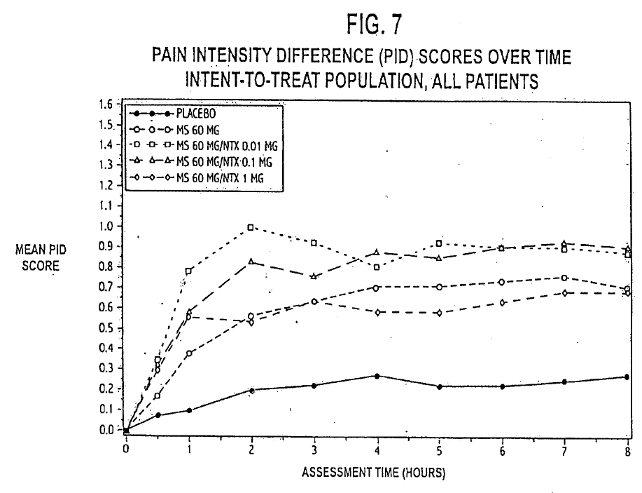

The hourly pain intensity difference (PID) data presented in Table 10 and FIG. 7. The hourly PID scores for the placebo treatment were generally flat while the hourly PD scores generally improved over time for the active treatment groups. The mean scores for the morphine and morphine/naltrexone groups were higher than the mean PID scores for the placebo group at each assessment time. The means for the low-dose and mid-dose groups were greater than the means for high-dose and placebo groups. Comparable pain relief as measured by PID scores was observed (see, e.g., 2-3 hours) in the MS alone group and the high-dose (1.0 mg NTX) combination group (FIG. 7). Highest pain relief as measured by PID scores was observed for the low-dose (0.01 mg NTX) combination group.

TABLE 10
Pain Intensity Difference (PID) Scores [1]
Intent-To-Treat Population

| PAIN RELIEF SCORE (PR) | | | | | | | | | |
|---|---|---|---|---|---|---|---|---|---|
| TREATMENT | N | MEAN | SD | MIN | MEDIAN | MAX | SOURCE | P-VALUE [2] | P-VALUE [3] |

30 MINUTES

| | | | | | | | | | |
|---|---|---|---|---|---|---|---|---|---|
| A) Placebo | 40 | 0.08 | 0.572 | −1 | 0.00 | 1 | TRT | 0.367 | 0.317 |
| B) MS 60 mg | 41 | 0.17 | 0.667 | −1 | 0.00 | 2 | BASEPI | N/A | <0.001*** |
| C) MS 60 mg/NTX 0.01 mg | 41 | 0.34 | 0.762 | −1 | 0.00 | 2 | BASEPI * TRT | N/A | 0.854 |
| D) MS 60 mg/NTX 0.1 mg | 41 | 0.32 | 0.650 | −1 | 0.00 | 2 | B-A | N/D | N/D |
| E) MS 60 mg/NTX 1 mg | 41 | 0.29 | 0.782 | −1 | 0.00 | 2 | C-A | N/D | N/D |
| | | | | | | | D-A | N/D | N/D |
| | | | | | | | E-A | N/D | N/D |
| | | | | | | | C-B | N/D | N/D |
| | | | | | | | D-B | N/D | N/D |
| | | | | | | | E-B | N/D | N/D |

1 HOUR

| | | | | | | | | | |
|---|---|---|---|---|---|---|---|---|---|
| A) Placebo | 40 | 0.10 | 0.744 | −1 | 0.00 | 2 | TRT | 0.11* | 0.007** |
| B) MS 60 mg | 41 | 0.38 | 0.886 | −1 | 0.00 | 2 | BASEPI | N/A | <0.001*** |
| C) MS 60 mg/NTX 0.01 mg | 41 | 0.78 | 1.013 | −1 | 1.00 | 3 | BASEPI * TRT | N/A | 0.361 |
| D) MS 60 mg/NTX 0.1 mg | 41 | 0.59 | 0.836 | −1 | 0.00 | 2 | B-A | 0.164 | 0.131 |
| E) MS 60 mg/NTX 1 mg | 41 | 0.56 | 0.950 | −1 | 0.00 | 2 | C-A | <0.001*** | <0.001*** |
| | | | | | | | D-A | 0.015* | 0.008** |
| | | | | | | | E-A | 0.020* | 0.012* |
| | | | | | | | C-B | 0.041* | 0.026* |
| | | | | | | | D-B | 0.289 | 0.250 |
| | | | | | | | E-B | 0.348 | 0.309 |

2 HOURS

| | | | | | | | | | |
|---|---|---|---|---|---|---|---|---|---|
| A) Placebo | 40 | 0.20 | 0.648 | −1 | 0.00 | 2 | TRT | 0.001** | <0.001*** |
| B) MS 60 mg | 41 | 0.56 | 1.001 | −1 | 1.00 | 3 | BASEPI | N/A | <0.001*** |
| C) MS 60 mg/NTX 0.01 mg | 41 | 1.00 | 1.000 | −1 | 1.00 | 3 | BASEPI * TRT | N/A | 0.042* |
| D) MS 60 mg/NTX 0.1 mg | 41 | 0.83 | 0.834 | −1 | 1.00 | 2 | B-A | 0.080 | 0.052 |
| E) MS 60 mg/NTX 1 mg | 41 | 0.54 | 1.075 | −1 | 0.00 | 2 | C-A | <0.001*** | <0.001*** |
| | | | | | | | D-A | 0.002** | <0.001*** |
| | | | | | | | E-A | 0.103 | 0.069 |
| | | | | | | | C-B | 0.032* | 0.017* |
| | | | | | | | D-B | 0.190 | 0.145 |
| | | | | | | | E-B | 0.905 | 0.894 |

3 HOURS

| | | | | | | | | | |
|---|---|---|---|---|---|---|---|---|---|
| A) Placebo | 40 | 0.23 | 0.660 | −1 | 0.00 | 2 | TRT | 0.031* | 0.021* |
| B) MS 60 mg | 41 | 0.63 | 1.067 | −1 | 1.00 | 3 | BASEPI | N/A | <0.001*** |
| C) MS 60 mg/NTX 0.01 mg | 41 | 0.93 | 1.081 | −1 | 1.00 | 3 | BASEPI * TRT | N/A | 0.025* |
| D) MS 60 mg/NTX 0.1 mg | 41 | 0.76 | 0.888 | −1 | 1.00 | 3 | B-A | 0.066 | 0.043* |
| E) MS 60 mg/NTX 1 mg | 41 | 0.63 | 1.199 | −1 | 0.00 | 3 | C-A | 0.001** | <0.001*** |
| | | | | | | | D-A | 0.017* | 0.009** |
| | | | | | | | E-A | 0.066 | 0.043* |
| | | | | | | | C-B | 0.185 | 0.145 |
| | | | | | | | D-B | 0.580 | 0.543 |
| | | | | | | | E-B | 1.000 | 1.000 |

4 HOURS

| | | | | | | | | | |
|---|---|---|---|---|---|---|---|---|---|
| A) Placebo | 40 | 0.28 | 0.751 | −1 | 0.00 | 2 | TRT | 0.078 | 0.035* |
| B) MS 60 mg | 41 | 0.71 | 1.146 | −1 | 1.00 | 3 | BASEPI | N/A | <0.001*** |
| C) MS 60 mg/NTX 0.01 mg | 41 | 0.80 | 0.954 | −1 | 1.00 | 3 | BASEPI * TRT | N/A | 0.010* |
| D) MS 60 mg/NTX 0.1 mg | 41 | 0.88 | 0.980 | −1 | 1.00 | 3 | B-A | N/D | 0.039* |
| E) MS 60 mg/NTX 1 mg | 41 | 0.59 | 1.245 | −1 | 0.00 | 3 | C-A | N/D | 0.011* |
| | | | | | | | D-A | N/D | 0.004** |
| | | | | | | | E-A | N/D | 0.138 |
| | | | | | | | C-B | N/D | 0.638 |
| | | | | | | | D-B | N/D | 0.411 |
| | | | | | | | E-B | N/D | 0.556 |

5 HOURS

| | | | | | | | | | |
|---|---|---|---|---|---|---|---|---|---|
| A) Placebo | 40 | 0.23 | 0.660 | −1 | 0.00 | 2 | TRT | 0.24* | 0.011* |
| B) MS 60 mg | 41 | 0.71 | 1.167 | −1 | 1.00 | 3 | BASEPI | N/A | <0.001*** |
| C) MS 60 mg/NTX 0.01 mg | 41 | 0.93 | 1.058 | −1 | 1.00 | 3 | BASEPI * TRT | N/A | 0.024* |
| D) MS 60 mg/NTX 0.1 mg | 41 | 0.85 | 0.989 | −1 | 1.00 | 3 | B-A | 0.038* | 0.025* |
| E) MS 60 mg/NTX 1 mg | 41 | 0.59 | 1.224 | −1 | 0.00 | 3 | C-A | 0.002** | 0.001** |
| | | | | | | | D-A | 0.007** | 0.003** |
| | | | | | | | E-A | 0.120 | 0.093 |
| | | | | | | | C-B | 0.340 | 0.302 |
| | | | | | | | D-B | 0.524 | 0.491 |
| | | | | | | | E-B | 0.596 | 0.566 |

6 HOURS

| | | | | | | | | | |
|---|---|---|---|---|---|---|---|---|---|
| A) Placebo | 40 | 0.23 | 0.660 | −1 | 0.00 | 2 | TRT | 0.032* | 0.016* |
| B) MS 60 mg | 41 | 0.73 | 1.162 | −1 | 1.00 | 2 | BASEPI | N/A | <0.001*** |
| C) MS 60 mg/NTX 0.01 mg | 41 | 0.90 | 1.114 | −1 | 1.00 | 3 | BASEPI * TRT | N/A | 0.013* |
| D) MS 60 mg/NTX 0.1 mg | 41 | 0.99 | 1.044 | −1 | 1.00 | 3 | B-A | 0.035* | 0.021* |
| E) MS 60 mg/NTX 1 mg | 41 | 0.63 | 1.299 | −1 | 0.00 | 3 | C-A | 0.005** | 0.002** |
| | | | | | | | D-A | 0.005** | 0.002** |
| | | | | | | | E-A | 0.089 | 0.063 |
| | | | | | | | C-B | 0.474 | 0.433 |
| | | | | | | | D-B | 0.474 | 0.433 |
| | | | | | | | E-B | 0.682 | 0.654 |

7 HOURS

| | | | | | | | | | |
|---|---|---|---|---|---|---|---|---|---|
| A) Placebo | 40 | 0.25 | 0.707 | −1 | 0.00 | 2 | TRT | 0.052 | 0.027* |
| B) MS 60 mg | 41 | 0.76 | 1.220 | −1 | 1.00 | 3 | BASEPI | N/A | <0.001*** |
| C) MS 60 mg/NTX 0.01 mg | 41 | 0.90 | 1.136 | −1 | 1.00 | 3 | BASEPI * TRT | N/A | 0.017* |
| D) MS 60 mg/NTX 0.1 mg | 41 | 0.93 | 1.058 | −1 | 1.00 | 3 | B-A | N/D | 0.027* |
| E) MS 60 mg/NTX 1 mg | 41 | 0.68 | 1.368 | −1 | 0.00 | 3 | C-A | N/D | 0.004** |
| | | | | | | | D-A | N/D | 0.003** |
| | | | | | | | E-A | N/D | 0.059 |
| | | | | | | | C-B | N/D | 0.519 |
| | | | | | | | D-B | N/D | 0.452 |
| | | | | | | | E-B | N/D | 0.747 |

8 HOURS

| | | | | | | | | | |
|---|---|---|---|---|---|---|---|---|---|
| A) Placebo | 40 | 0.28 | 0.784 | −1 | 0.00 | 3 | TRT | 0.095 | 0.056 |
| B) MS 60 mg | 41 | 0.71 | 1.230 | −1 | 1.00 | 3 | BASEPI | N/A | <0.001*** |
| C) MS 60 mg/NTX 0.01 mg | 41 | 0.88 | 1.144 | −1 | 1.00 | 3 | BASEPI * TRT | N/A | 0.029* |
| D) MS 60 mg/NTX 0.1 mg | 41 | 0.90 | 1.044 | −1 | 1.00 | 3 | B-A | N/D | N/D |
| E) MS 60 mg/NTX 1 mg | 41 | 0.68 | 1.350 | −1 | 0.00 | 3 | C-A | N/D | N/D |
| | | | | | | | D-A | N/D | N/D |
| | | | | | | | E-A | N/D | N/D |
| | | | | | | | C-B | N/D | N/D |
| | | | | | | | D-B | N/D | N/D |
| | | | | | | | E-B | N/D | N/D |

[1] PAIN INTENSITY SCORES: 0 = NONE, 1 = MILD, 2 = MODERATE, 3 = SEVERE.
[2] FROM ONE-WAY ANALYSIS OF VARIANCE AND FISHER'S PROTECTED LEAST SIGNIFICANT DIFFERENCE TEST.
[3] FROM TWO-WAY ANALYSIS OF VARIANCE WITH BASELINE PAIN INTENSITY AS A BLOCKING FACTOR AND FISHER'S PROTECTED LEAST SIGNIFICANT DIFFERENCE TEST.
*, **, ***P-VALUE <=0.05, <=0.01, or <=0.001 RESPECTIVELY.
N/A: NOT APPLICABLE, N/D: NOT DONE (BECAUSE OVERALL P-VALUE NOT SIGNIFICANT).

The mean MAXPAR scores presented in Table 11A were different among treatment groups. The mean MAXPAR scores were highest for the low-dose and mid-dose groups compared to all other groups. The mean scores for the low-dose and mid-dose groups were greater than the mean score for the morphine group, which in turn, was greater than the mean score for the placebo group. The mean PEAKPID scores presented in Table 11B were different among treatment groups, and were greater for the morphine/naltrexone groups compared to the placebo group. Compared to all other groups, the mean PEAKPID scores were higher for the low-dose and mid-dose groups.

TABLE 11A
Maximum Pain Relief Scores (MAXPAR)
Intent-To-Treat Population

| MAXIMUM PAIN RELIEF SCORE (PR) | | | | | | | | | |
|---|---|---|---|---|---|---|---|---|---|
| TREATMENT | N | MEAN | SD | MIN | MEDIAN | MAX | SOUCRE | P-VALUE [1] | P-VALUE [2] |
| A) Placebo | 40 | 1.10 | 1.355 | 0.0 | 0.5 | 4.0 | TRT | 0.002** | 0.004** |
| B) MS 60 mg | 41 | 1.95 | 1.532 | 0.0 | 3.0 | 4.0 | BASEPI | N/A | 0.569 |
| C) MS 60 mg/NTX 0.01 mg | 41 | 2.39 | 1.531 | 0.0 | 3.0 | 4.0 | BASEPI * TRT | N/A | 0.100 |
| D) MS 60 mg/NTX 0.1 mg | 41 | 2.10 | 1.463 | 0.0 | 2.0 | 4.0 | B-A | 0.011* | 0.011* |
| E) MS 60 mg/NTX 1 mg | 41 | 1.71 | 1.632 | 0.0 | 1.0 | 4.0 | C-A | <0.001*** | <0.001*** |
| | | | | | | | D-A | 0.003** | 0.003** |
| | | | | | | | E-A | 0.071 | 0.068 |
| | | | | | | | C-B | 0.188 | 0.184 |
| | | | | | | | D-B | 0.660 | 0.657 |
| | | | | | | | E-B | 0.464 | 0.460 |

[1] FROM ONE-WAY ANALYSIS OF VARIANCE AND FISHER'S PROTECTED LEAST SIGNIFICANT DIFFERENCE TEST.
[2] FROM TWO-WAY ANALYSIS OF VARIANCE WITH BASELINE PAIN INTENSITY AS A BLOCKING FACTOR AND FISHER'S PROTECTED LEAST SIGNIFICANT DIFFERENCE TEST.
*, **, ***P-VALUE <=0.05, <=0.01, or <=0.001 RESPECTIVELY.
N/A: NOT APPLICABLE

TABLE 11B
Peak Pain Intensity Difference (PEAKPID)
Intent-To-Treat Population

| PEAK PAIN INTENSITY DIFFERENCE | | | | | | | | | |
|---|---|---|---|---|---|---|---|---|---|
| TREATMENT | N | MEAN | SD | MIN | MEDIAN | MAX | SOURCE | P-VALUE [1] | P-VALUE [2] |
| A) Placebo | 40 | 0.53 | 0.877 | −1 | 0.0 | 3 | TRT | 0.007** | 0.004** |
| B) MS 60 mg | 41 | 1.10 | 1.068 | −1 | 1.0 | 3 | BASEPI | N/A | <0.001*** |
| C) MS 60 mg/NTX 0.01 mg | 41 | 1.41 | 1.140 | −1 | 2.0 | 3 | BASEPI * TRT | N/A | 0.073 |
| D) MS 60 mg/NTX 0.1 mg | 41 | 1.17 | 1.022 | −1 | 1.0 | 3 | B-A | 0.019* | 0.011* |
| E) MS 60 mg/NTX 1 mg | 41 | 1.00 | 1.304 | −1 | 1.0 | 3 | C-A | <0.001*** | <0.001*** |
| | | | | | | | D-A | 0.008** | 0.004** |
| | | | | | | | E-A | 0.051 | 0.034* |
| | | | | | | | C-B | 0.190 | 0.154 |
| | | | | | | | D-B | 0.761 | 0.742 |
| | | | | | | | E-B | 0.686 | 0.660 |

[1] FROM ONE-WAY ANALYSIS OF VARIANCE AND FISHER'S PROTECTED LEAST SIGNIFICANT DIFFERENCE TEST.
[2] FROM TWO-WAY ANALYSIS OF VARIANCE WITH BASELINE PAIN INTENSITY AS A BLOCKING FACTOR AND FISHER'S PROTECTED LEAST SIGNIFICANT DIFFERENCE TEST.
*, **, ***P-VALUE <=0.05, <=0.01, or <=0.001 RESPECTIVELY.
N/A: NOT APPLICABLE

Table 12 presents the summary and analysis of global evaluations. The placebo treatment had the highest number of subjects who had poor global evaluation scores based on subject evaluation. The profiles of the global evaluations scores are based on subjects' evaluations. Analyses of global evaluations for the evaluable subgroup also yielded similar results.

TABLE 12
Global Evaluation of Study Medication
Intent-To-Treat Population

| | | | VERY | | | | | | | |
|---|---|---|---|---|---|---|---|---|---|---|
| | | EXCELLENT | GOOD | GOOD | FAIR | POOR | MEAN | | P-VALUE | P-VALUE |
| TREATMENT | N | (1) | (2) | (3) | (4) | (5) | (SE) | SOURCE | [1] | [2] |
| A) Placebo | 40 | 0 (0.0%) | 6 (15.0%) | 4 (10.0%) | 2 (5.0%) | 28 (70.0%) | 0.7 (1.16) | TRT | 0.004** | 0.010* |
| B) MS 60 mg | 41 | 3 (7.3%) | 10 (24.4%) | 8 (19.5%) | 3 (7.3%) | 17 (41.5%) | 1.5 (1.43) | BASEPI | N/A | 0.958 |
| C) MS 60 | 41 | 3 (7.3%) | 14 (34.1%) | 9 (22.0%) | 3 (7.3%) | 11 (26.8%) | 1.9 (1.36) | BASEPI * TRT | N/A | 0.029* |
| mg/NTX 0.01 mg | | | | | | | | | | |
| D) MS 60 | 41 | 3 (7.3%) | 9 (22.0%) | 7 (17.1%) | 8 (19.5%) | 14 (34.1%) | 1.5 (1.36) | B-A | 0.008** | 0.008** |
| mg/NTX 0.1 mg | | | | | | | | | | |
| E) MS 60 | 41 | 4 (9.8%) | 5 (12.2%) | 10 (24.4%) | 2 (4.9%) | 20 (48.8%) | 1.3 (1.44) | C-A | <0.001*** | <0.001*** |
| mg/NTX 1 mg | | | | | | | | D-A | 0.007** | 0.008 |
| | | | | | | | | E-A | 0.045 | 0.047 |
| | | | | | | | | C-B | 0.214 | 0.190 |
| | | | | | | | | D-B | 1.000 | 1.000 |
| | | | | | | | | E-B | 0.536 | 0.509 |

[1] FROM ONE-WAY ANALYSIS OF VARIANCE AND ITS FISHER'S PROTECTED LEAST SIGNIFICANT DIFFERENCE TEST.
[2] FROM TWO-WAY ANALYSIS OF VARIANCE WITH BASELINE PAIN INTENSITY AS A BLOCKING FACTOR AND ITS FISHER'S PROTECTED LEAST SIGNIFICANT DIFFERENCE TEST.
*, **, ***P-VALUE <=0.05, +0.01, OR <=0.001 RESPECTIVELY
N/A: NOT APPLICABLE

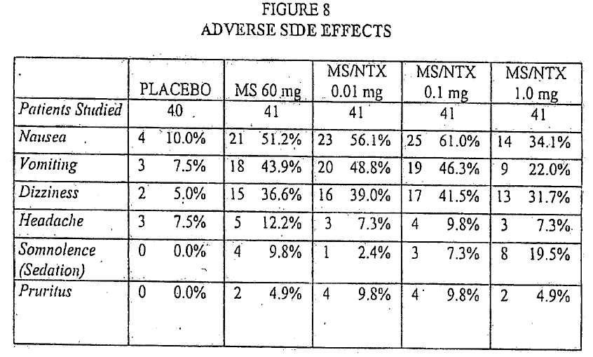

The majority of adverse events reported were categorized as digestive (nausea or vomiting) or nervous system (dizziness or somnolence) as further shown in Tables 13A or 13B. FIG. 8 represents a summary of exemplary adverse side effects attenuated according to methods and compositions of the invention.

TABLE 13A
Adverse Events By Body System And Severity
Safety Population

| Body System | | Total | No. Of | | | Total | | | |
|---|---|---|---|---|---|---|---|---|---|
| Adverse Events | | No. Of | Subjects | | | No. Of | Severity [2] | | |
| (Costart English) | Treatment | Subjects | W/Event | Source | P-Value [1] | Events | Mild | Moderate | Severe |

Total Number Of Events

| | | | | | | | | | |
|---|---|---|---|---|---|---|---|---|---|
| Adverse Events | A) PLACEBO | 40 | 11 (27.5%) | TRT | <0.001*** | 17 | 7 (41.2%) | 5 (29.4%) | 5 (29.4%) |
| (All Body | B) MS 60 MG | 41 | 35 (85.4%) | A-B | <0.001*** | 82 | 28 (34.1%) | 32 (39.0%) | 22 (26.8%) |
| Systems) | C) MS 60 MG/NTX 0.01 MG | 41 | 36 (87.8%) | A-C | <0.001*** | 93 | 22 (23.7%) | 40 (43.0%) | 31 (33.3%) |
| | D) MS 60 MG/NTX 0.1 MG | 41 | 37 (90.2%) | A-D | <0.001*** | 102 | 28 (27.5%) | 40 (39.2%) | 34 (33.3%) |
| | E) MS 60 MG/NTX 1 MG | 41 | 31 (75.6%) | A-E | <0.001*** | 64 | 31 (48.4%) | 22 (34.4%) | 11 (17.2%) |

Body As A Whole

| | | | | | | | | | |
|---|---|---|---|---|---|---|---|---|---|
| All Events | A) PLACEBO | 40 | 4 (10.0%) | TRT | 0.675 | 4 | 1 (25.0%) | 3 (75.0%) | 0 |
| | B) MS 60 MG | 41 | 6 (14.6%) | | | 7 | 4 (57.1%) | 3 (42.9%) | 0 |
| | C) MS 60 MG/NTX 0.01 MG | 41 | 8 (19.5%) | | | 8 | 2 (25.0%) | 4 (50.0%) | 2 (25.0%) |
| | D) MS 60 MG/NTX 0.1 MG | 41 | 7 (17.1%) | | | 10 | 3 (30.0%) | 5 (50.0%) | 2 (20.0%) |
| | E) MS 60 MG/NTX 1 MG | 41 | 4 (9.8%) | | | 4 | 2 (50.0%) | 2 (50.0%) | 0 |
| Abdominal | A) PLACEBO | 40 | 0 | TRT | 0.512 | 0 | 0 | 0 | 0 |
| Pain | B) MS 60 MG | 41 | 0 | | | 0 | 0 | 0 | 0 |
| | C) MS 60 MG/NTX 0.01 MG | 41 | 2 (4.9%) | | | 2 | 0 | 0 | 2 (100.0%) |
| | D) MS 60 MG/NTX 0.1 MG | 41 | 1 (2.4%) | | | 1 | 0 | 0 | 1 (100.0%) |
| | E) MS 60 MG/NTX 1 MG | 41 | 0 | | | 0 | 0 | 0 | 0 |
| Asthenia | A) PLACEBO | 40 | 0 | TRT | 1.000 | 0 | 0 | 0 | 0 |
| | B) MS 60 MG | 41 | 1 (2.4%) | | | 1 | 1 (100.0%) | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 1 (2.4%) | | | 1 | 0 | 1 (100.0%) | 0 |
| | E) MS 60 MG/NTX 1 MG | 41 | 1 (2.4%) | | | 1 | 1 (100.0%) | 0 | 0 |
| Fever | A) PLACEBO | 40 | 1 (2.5%) | TRT | 0.196 | 1 | 0 | 1 (100.0%) | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Headache | A) PLACEBO | 40 | 3 (7.5%) | TRT | 0.960 | 3 | 1 (33.3%) | 2 (66.7%) | 0 |
| | B) MS 60 MG | 41 | 5 (12.2%) | | | 5 | 2 (40.0%) | 3 (60.0%) | 0 |
| | C) MS 60 MG/NTX 0.01 MG | 41 | 3 (7.3%) | | | 3 | 2 (66.7%) | 1 (33.3%) | 0 |
| | D) MS 60 MG/NTX 0.1 MG | 41 | 4 (9.8%) | | | 6 | 2 (33.3%) | 3 (50.0%) | 1 (16.7%) |
| | E) MS 60 MG/NTX 1 MG | 41 | 3 (7.3%) | | | 3 | 1 (33.3%) | 2 (66.7%) | 0 |
| Injection Site | A) PLACEBO | 40 | 0 | TRT | 1.000 | 0 | 0 | 0 | 0 |
| Hemorrhage | B) MS 60 MG | 41 | 1 (2.4%) | | | 1 | 1 (100.0%) | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Overdose | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Pain | A) PLACEBO | 40 | 0 | TRT | 0.512 | 0 | 0 | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 2 (4.9%) | | | 2 | 1 (50.0%) | 1 (50.0%) | 0 |
| | E) MS 60 MG/NTX 1 MG | 41 | 0 | | | 0 | 0 | 0 | 0 |

Cardiovascular

| | | | | | | | | | |
|---|---|---|---|---|---|---|---|---|---|
| All Events | A) PLACEBO | 40 | 0 | TRT | 0.124 | 0 | 0 | 0 | 0 |
| | B) MS 60 MG | 41 | 3 (7.3%) | | | 3 | 2 (66.7%) | 1 (33.3%) | 0 |
| | C) MS 60 MG/NTX 0.01 MG | 41 | 4 (9.8%) | | | 4 | 2 (50.0%) | 1 (25.0%) | 1 (25.0%) |
| | D) MS 60 MG/NTX 0.1 MG | 41 | 5 (12.2%) | | | 5 | 2 (40.0%) | 3 (60.0%) | 0 |
| | E) MG 60 MG/NTX 1 MG | 41 | 1 (2.4%) | | | 1 | 1 (100.0%) | 0 | 0 |
| Hemorrhage | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 1 (2.4%) | | | 1 | 0 | 1 (100.0%) | 0 |
| | E) MG 60 MG/NTX 1 MG | 41 | 0 | | | 0 | 0 | 0 | 0 |
| Hypertension | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 1 (2.4%) | | | 1 | 1 (100.0%) | 0 | 0 |
| | E) MG 60 MG/NTX 1 MG | 41 | 0 | | | 0 | 0 | 0 | 0 |
| Vasodilatation | A) PLACEBO | 40 | 0 | TRT | 0.257 | 0 | 0 | 0 | 0 |
| | B) MS 60 MG | 41 | 3 (7.3%) | | | 3 | 2 (66.7%) | 1 (33.3%) | 0 |
| | C) MS 60 MG/NTX 0.01 MG | 41 | 4 (9.8%) | | | 4 | 2 (50.0%) | 1 (25.0%) | 1 (25.0%) |
| | D) MS 60 MG/NTX 0.1 MG | 41 | 3 (7.3%) | | | 3 | 1 (33.3%) | 2 (66.7%) | 0 |
| | E) MS 60 MG/NTX 1 MG | 41 | 1 (2.4%) | | | 1 | 1 (100.0%) | 0 | 0 |

Digestive

| | | | | | | | | | |
|---|---|---|---|---|---|---|---|---|---|
| All Events | A) PLACEBO | 40 | 5 (12.5%) | TRT | <0.001*** | 8 | 1 (12.5%) | 2 (25.0%) | 5 (62.5%) |
| | B) MS 60 MG | 41 | 23 (56.1%) | A-B | <0.001*** | 40 | 6 (15.0%) | 14 (35.0%) | 20 (50.0%) |
| | C) MS 60 MG/NTX 0.01 MG | 41 | 25 (61.0%) | A-C | <0.001*** | 46 | 7 (15.2%) | 15 (32.6%) | 24 (52.2%) |
| | D) MS 60 MG/NTX 0.1 MG | 41 | 29 (70.7%) | A-D | <0.001*** | 47 | 8 (17.0%) | 12 (25.5%) | 27 (57.4%) |
| | E) MS 60 MG/NTX 1 MG | 41 | 16 (39.0%) | A-E | <0.010* | 25 | 6 (24.0%) | 8 (32.0%) | 11 (44.0%) |
| | | | | D-E | <0.007** | | | | |
| Diarrhea | A) PLACEBO | 40 | 0 | TRT | 0.196 | 0 | 0 | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 2 (4.9%) | | | 2 | 1 (50.0%) | 1 (50.0%) | 0 |
| | E) MG 60 MG/NTX 1 MG | 41 | 0 | | | 0 | 0 | 0 | 0 |
| Dyspepsia | A) PLACEBO | 40 | 1 (2.5%) | TRT | 0.512 | 1 | 1 (100.0%) | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 1 (2.4%) | | | 1 | 1 (100.0%) | 0 | 0 |
| Nausea | A) PLACEBO | 40 | 4 (10.0%) | TRT | <0.001*** | 4 | 0 | 2 (50.0%) | 2 (50.0%) |
| | B) MS 60 MG | 41 | 21 (51.2%) | A-B | <0.001*** | 22 | 6 (27.3%) | 14 (63.6%) | 2 (9.1%) |
| | C) MS 60 MG/NTX 0.01 MG | 41 | 23 (56.1%) | A-C | <0.001*** | 26 | 7 (26.9%) | 15 (57.7%) | 4 (15.4%) |
| | D) MS 60 MG/NTX 0.1 MG | 41 | 25 (61.0%) | A-D | <0.001*** | 26 | 7 (26.9%) | 11 (42.3%) | 8 (30.8%) |
| | E) MS 60 MG/NTX 1 MG | 41 | 14 (34.1%) | A-E | <0.014* | 15 | 5 (33.3%) | 8 (53.3%) | 2 (13.3%) |
| | | | | D-E | <0.026* | | | | |
| Vomiting | A) PLACEBO | 40 | 3 (7.5%) | TRT | <0.001*** | 3 | 0 | 0 | 3 (100.0%) |
| | B) MS 60 MG | 41 | 18 (43.9%) | A-B | <0.001*** | 18 | 0 | 0 | 18 (100.0%) |
| | C) MS 60 MG/NTX 0.01 MG | 41 | 20 (48.8%) | A-C | <0.001*** | 20 | 0 | 0 | 20 (100.0%) |
| | D) MS 60 MG/NTX 0.1 MG | 41 | 19 (46.3%) | A-D | <0.001*** | 19 | 0 | 0 | 19 (100.0%) |
| | E) MS 60 MG/NTX 1 MG | 41 | 9 (22.0%) | A-E | <0.020* | 9 | 0 | 0 | 9 (100.0%) |
| | | | | D-E | <0.035* | | | | |

Musculoskeletal

| | | | | | | | | | |
|---|---|---|---|---|---|---|---|---|---|
| All Events | A) PLACEBO | 40 | 0 | TRT | 1.000 | 0 | 0 | 0 | 0 |
| | B) MS 60 MG | 41 | 1 (2.4%) | | | 1 | 0 | 1 (100.0%) | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Myalgia | A) PLACEBO | 40 | 0 | TRT | 1.000 | 0 | 0 | 0 | 0 |
| | B) MS 60 MG | 41 | 1 (2.4%) | | | 1 | 0 | 1 (100.0%) | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |

Nervous System

| | | | | | | | | | |
|---|---|---|---|---|---|---|---|---|---|
| All Events | A) PLACEBO | 40 | 2 (5.0%) | TRT | <0.001*** | 2 | 2 (100.0%) | 0 | 0 |
| | B) MS 60 MG | 41 | 18 (43.9%) | A-B | <0.001*** | 24 | 11 (45.8%) | 11 (45.8%) | 2 (8.3%) |
| | C) MS 60 MG/NTX 0.01 MG | 41 | 22 (53.7%) | A-C | <0.001*** | 25 | 6 (24.0%) | 15 (60.0%) | 4 (16.0%) |
| | D) MS 60 MG/NTX 0.1 MG | 41 | 22 (53.7%) | A-D | <0.001*** | 29 | 9 (31.0%) | 15 (51.7%) | 5 (17.2%) |
| | E) MS 60 MG/NTX 1 MG | 41 | 20 (48.8%) | A-E | <0.001*** | 26 | 16 (61.5%) | 10 (38.5%) | 0 |
| Anxiety | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 1 (2.4%) | | | 1 | 1 (100.0%) | 0 | 0 |
| | E) MS 60 MG/NTX 1 MG | 41 | 0 | | | 0 | 0 | 0 | 0 |
| Dizziness | A) PLACEBO | 40 | 2 (5.0%) | TRT | <0.001*** | 2 | 2 (100.0%) | 0 | 0 |
| | B) MS 60 MG | 41 | 15 (36.6%) | A-B | <0.001*** | 17 | 9 (52.9%) | 6 (35.3%) | 2 (11.8%) |
| | C) MS 60 MG/NTX 0.01 MG | 41 | 16 (39.0%) | A-C | <0.001*** | 16 | 5 (31.3%) | 9 (56.3%) | 2 (12.5%) |
| | D) MS 60 MG/NTX 0.1 MG | 41 | 17 (41.5%) | A-D | <0.001*** | 20 | 6 (30.0%) | 10 (50.0%) | 4 (20.0%) |
| | E) MS 60 MG/NTX 1 MG | 41 | 13 (31.7%) | A-E | <0.003** | 13 | 8 (61.5%) | 5 (38.5%) | 0 |
| Dry Mouth | A) PLACEBO | 40 | 0 | TRT | 0.196 | 0 | 0 | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 2 (4.9%) | | | 2 | 1 (50.0%) | 1 (50.0%) | 0 |
| Euphoria | A) PLACEBO | 40 | 0 | TRT | 0.005** | 0 | 0 | 0 | 0 |
| | B) MS 60 MG | 41 | 0 | | | 0 | 0 | 0 | 0 |
| | C) MS 60 MG/NTX 0.01 MG | 41 | 5 (12.2%) | | | 5 | 0 | 4 (80.0%) | 1 (20.0%) |
| | D) MS 60 MG/NTX 0.1 MG | 41 | 2 (4.9%) | | | 2 | 1 (50.0%) | 1 (50.0%) | 0 |
| | E) MS 60 MG/NTX 1 MG | 41 | 0 | | | 0 | 0 | 0 | 0 |
| Hallucinations | A) PLACEBO | 40 | 0 | TRT | 1.000 | 0 | 0 | 0 | 0 |
| | B) MS 60 MG | 41 | 1 (2.4%) | | | 1 | 0 | 1 (100.0%) | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Hypertonia | A) PLACEBO | 40 | 0 | TRT | 1.000 | 0 | 0 | 0 | 0 |
| | B) MS 60 MG | 41 | 1 (2.4%) | | | 1 | 0 | 1 (100.0%) | 0 |
| | C) MS 60 MG/NTX 0.01 MG | 41 | 1 (2.4%) | | | 1 | 0 | 0 | 1 (100.0%) |
| | D) MS 60 MG/NTX 0.1 MG | 41 | 1 (2.4%) | | | 1 | 0 | 1 (100.0%) | 0 |
| | E) MS 60 MG/NTX 1 MG | 41 | 0 | | | 0 | 0 | 0 | 0 |
| Paresthesia | A) PLACEBO | 40 | 0 | TRT | 0.802 | 0 | 0 | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 1 (100.0%) | 0 | 0 |
| | D) MS 60 MG/NTX 0.1 MG | 41 | 1 (2.4%) | | | 1 | 0 | 1 (100.0%) | 0 |
| | E) MS 60 MG/NTX 1 MG | 41 | 2 (4.9%) | | | 2 | 1 (50.0%) | 1 (50.0%) | 0 |
| Somnolence | A) PLACEBO | 40 | 0 | TRT | 0.009** | 0 | 0 | 0 | 0 |
| | B) MS 60 MG | 41 | 4 (9.8%) | A-E | 0.005** | 4 | 2 (50.0%) | 2 (50.0%) | 0 |
| | C) MS 60 MG/NTX 0.01 MG | 41 | 1 (2.4%) | C-E | 0.029* | 1 | 0 | 1 (100.0%) | 0 |
| | D) MS 60 MG/NTX 0.1 MG | 41 | 3 (7.3%) | | | 3 | 0 | 2 (66.7%) | 1 (33.3%) |
| | E) MS 60 MG/NTX 1 MG | 41 | 8 (19.5%) | | | 8 | 5 (62.5%) | 3 (37.5%) | 0 |
| Tremor | A) PLACEBO | 40 | 0 | TRT | 1.000 | 0 | 0 | 0 | 0 |
| | B) MS 60 MG | 41 | 1 (2.4%) | | | 1 | 0 | 1 (100.0%) | 0 |
| | C) MS 60 MG/NTX 0.01 MG | 41 | 0 | | | 0 | 0 | 0 | 0 |
| | D) MS 60 MG/NTX 0.1 MG | 41 | 1 (2.4%) | | | 1 | 1 (100.0%) | 0 | 0 |
| | E) MS 60 MG/NTX 1 MG | 41 | 1 (2.4%) | | | 1 | 1 (100.0%) | 0 | 0 |

Respiratory

| | | | | | | | | | |
|---|---|---|---|---|---|---|---|---|---|
| All Events | A) PLACEBO | 40 | 2 (5.0%) | TRT | 0.335 | 2 | 2 (100.0%) | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 0 | | | 0 | 0 | 0 | 0 |
| | E) MS 60 MG/NTX 1 MG | 41 | 1 (2.4%) | | | 1 | 1 (100.0%) | 0 | 0 |
| Dyspnea | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 1 (2.4%) | | | 1 | 1 (100.0%) | 0 | 0 |
| Epistaxis | A) PLACEBO | 40 | 1 (2.5%) | TRT | 0.512 | 1 | 1 (100.0%) | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Rhinitis | A) PLACEBO | 40 | 1 (2.5%) | TRT | 0.196 | 1 | 1 (100.0%) | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |

Skin/Appendages

| | | | | | | | | | |
|---|---|---|---|---|---|---|---|---|---|
| All Events | A) PLACEBO | 40 | 0 | TRT | 0.244 | 0 | 0 | 0 | 0 |
| | B) MS 60 MG | 41 | 4 (9.8%) | | | 4 | 2 (50.0%) | 2 (50.0%) | 0 |
| | C) MS 60 MG/NTX 0.01 MG | 41 | 4 (9.8%) | | | 5 | 2 (40.0%) | 3 (60.0%) | 0 |
| | D) MS 60 MG/NTX 0.1 MG | 41 | 4 (9.8%) | | | 4 | 0 | 4 (100.0%) | 0 |
| | E) MS 60 MG/NTX 1 MG | 41 | 4 (9.8%) | | | 5 | 3 (60.0%) | 2 (40.0%) | 0 |
| Puritus | A) PLACEBO | 40 | 0 | TRT | 0.264 | 0 | 0 | 0 | 0 |
| | B) MS 60 MG | 41 | 2 (4.9%) | | | 2 | 1 (50.0%) | 1 (50.0%) | 0 |
| | C) MS 60 MG/NTX 0.01 MG | 41 | 4 (9.8%) | | | 4 | 2 (50.0%) | 2 (50.0%) | 0 |
| | D) MS 60 MG/NTX 0.1 MG | 41 | 4 (9.8%) | | | 4 | 0 | 4 (100.0%) | 0 |
| | E) MS 60 MG/NTX 1 MG | 41 | 2 (4.9%) | | | 2 | 2 (100.0%) | 0 | 0 |
| Rash | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 0 | | | 0 | 0 | 0 | 0 |
| | E) MS 60 MG/NTX 1 MG | 41 | 1 (2.4%) | | | 1 | 0 | 1 (100.0%) | 0 |
| Sweating | A) PLACEBO | 40 | 0 | TRT | 0.223 | 0 | 0 | 0 | 0 |
| | B) MS 60 MG | 41 | 2 (4.9%) | | | 2 | 1 (50.0%) | 1 (50.0%) | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 2 (4.9%) | | | 2 | 1 (50.0%) | 1 (50.0%) | 0 |

Special Senses

| | | | | | | | | | |
|---|---|---|---|---|---|---|---|---|---|
| All Events | A) PLACEBO | 40 | 1 (2.5%) | TRT | 0.798 | 1 | 1 (100.0%) | 0 | 0 |
| | B) MS 60 MG | 41 | 2 (4.9%) | | | 2 | 2 (100.0%) | 0 | 0 |
| | C) MS 60 MG/NTX 0.01 MG | 41 | 3 (7.3%) | | | 3 | 3 (100.0%) | 0 | 0 |
| | D) MS 60 MG/NTX 0.1 MG | 41 | 4 (9.8%) | | | 4 | 3 (75.0%) | 1 (25.0%) | 0 |
| | E) MS 60 MG/NTX 1 MG | 41 | 2 (4.9%) | | | 2 | 2 (100.0%) | 0 | 0 |
| Conjunctivitis | A) PLACEBO | 40 | 1 (2.5%) | TRT | 0.798 | 1 | 1 (100.0%) | 0 | 0 |
| | B) MS 60 MG | 41 | 2 (4.9%) | | | 2 | 2 (100.0%) | 0 | 0 |
| | C) MS 60 MG/NTX 0.01 MG | 41 | 3 (7.3%) | | | 3 | 3 (100.0%) | 0 | 0 |
| | D) MS 60 MG/NTX 0.1 MG | 41 | 4 (9.8%) | | | 4 | 3 (75.0%) | 1 (25.0%) | 0 |
| | E) MS 60 MG/NTX 1 MG | 41 | 2 (4.9%) | | | 2 | 2 (100.0%) | 0 | 0 |

Urogenital

| | | | | | | | | | |
|---|---|---|---|---|---|---|---|---|---|
| All Events | A) PLACEBO | 40 | 0 | TRT | 0.278 | 0 | 0 | 0 | 0 |
| | B) MS 60 MG | 41 | 1 (2.4%) | | | 1 | 1 (100.0%) | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 3 (7.3%) | | | 3 | 3 (100.0%) | 0 | 0 |
| | E) MS 60 MG/NTX 1 MG | 41 | 0 | | | 0 | 0 | 0 | 0 |
| Dysuria | A) PLACEBO | 40 | 0 | TRT | 1.000 | 0 | 0 | 0 | 0 |
| | B) MS 60 MG | 41 | 0 | | | 0 | 0 | 0 | 0 |
| | C) MS 60 MG/NTX 0.01 MG | 41 | 0 | | | 0 | 0 | 0 | 0 |
| | D) MS 60 MG/NTX 0.1 MG | 41 | 1 (2.4%) | | | 1 | 1 (100.0%) | 0 | 0 |
| | E) MS 60 MG/NTX 1 MG | 41 | 0 | | | 0 | 0 | 0 | 0 |
| Metrorrhagia | A) PLACEBO | 40 | 0 | TRT | 1.000 | 0 | 0 | 0 | 0 |
| | B) MS 60 MG | 41 | 1 (2.4%) | | | 1 | 1 (100.0%) | 0 | 0 |
| | C) MS 60 MG/NTX 0.01 MG | 41 | 0 | | | 0 | 0 | 0 | 0 |
| | D) MS 60 MG/NTX 0.1 MG | 41 | 0 | | | 0 | 0 | 0 | 0 |
| | E) MS 60 MG/NTX 1 MG | 41 | 0 | | | 0 | 0 | 0 | 0 |
| Urinary | A) PLACEBO | 40 | 0 | TRT | 0.512 | 0 | 0 | 0 | 0 |
| Retention | B) MS 60 MG | 41 | 0 | | | 0 | 0 | 0 | 0 |
| | C) MS 60 MG/NTX 0.01 MG | 41 | 1 (2.4%) | | | 1 | 0 | 1 (100.0%) | 0 |
| | D) MS 60 MG/NTX 0.1 MG | 41 | 2 (4.9%) | | | 2 | 2 (100.0%) | 0 | 0 |
| | E) MS 60 MG/NTX 1 MG | 41 | 0 | | | 0 | 0 | 0 | 0 |

[1] P-VALUES ARE FROM FISHER'S EXACT TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
*, **, ***P-VALUE <=0.05, <=0.01, OR <=0.001 RESPECTIVELY.

TABLE 13B
SELECTED ADVERSE EVENTS
SAFETY POPULATION

| | | | NO. OF SUBJECTS WITH AEs | | | | FISHER'S EXACT P-VALUE [1] | | |
|---|---|---|---|---|---|---|---|---|---|
| | | | RELATED | | | | FOR AEs RELATED | | |
| ADVERSE | | TOTAL | | TO | WITH | | | TO | FOR |
| EVENT | | NO. OF | WITH | STUDY | SERIOUS | | | STUDY | SERIOUS |
| (ENGLISH) | TREATMENT | SUBJECTS | AEs | DRUG [2] | AEs | SOURCE | FOR AEs | DRUG [2] | AEs |
| DIZZINESS | A) PLACEBO | 40 | 2 (5.0%) | 2 (5.0%) | 0 (0.0%) | TREATMENT | <0.001*** | <0.001*** | N/A |
| | B) MS 60 MG | 41 | 15 (36.6%) | 15 (36.6%) | 0 (0.0%) | A-B | <0.001*** | <0.001*** | N/A |
| | C) MS 60 MG/NTX | 41 | 16 (39.0%) | 16 (39.0%) | 0 (0.0%) | A-C | <0.001*** | <0.001*** | N/A |
| | 0.01 MG | | | | | | | | |
| | D) MS 60 MG/NTX | 41 | 17 (41.5%) | 17 (41.5%) | 0 (0.0%) | A-D | <0.001*** | <0.001*** | N/A |
| | 0.1 MG | | | | | | | | |
| | E) MS 60 MG/NTX | 41 | 13 (31.7%) | 13 (31.7%) | 0 (0.0%) | A-E | 0.003** | 0.003** | N/A |
| | 1 MG | | | | | B-C | 1.000 | 1.000 | N/A |
| | | | | | | B-D | 0.821 | 0.821 | N/A |
| | | | | | | B-E | 0.816 | 0.816 | N/A |
| | | | | | | C-D | 1.000 | 1.000 | N/A |
| | | | | | | C-E | 0.644 | 0.644 | N/A |
| | | | | | | D-E | 0.491 | 0.491 | N/A |
| NAUSEA | A) PLACEBO | 40 | 4 (10.0%) | 3 (7.5%) | 0 (0.0%) | TREATMENT | <0.001*** | <0.001*** | N/A |
| | B) MS 60 MG | 41 | 21 (51.2%) | 21 (51.2%) | 0 (0.0%) | A-B | <0.001*** | <0.001*** | N/A |
| | C) MS 60 MG/NTX | 41 | 23 (56.1%) | 23 (56.1%) | 0 (0.0%) | A-C | <0.001*** | <0.001*** | N/A |
| | 0.01 MG | | | | | | | | |
| | D) MS 60 MG/NTX | 41 | 25 (61.0%) | 25 (61.0%) | 0 (0.0%) | A-D | <0.001*** | <0.001*** | N/A |
| | 0.1 MG | | | | | | | | |
| | E) MS 60 MG/NTX | 41 | 14 (34.1%) | 12 (29.3%) | 0 (0.0%) | A-E | 0.014* | 0.020* | N/A |
| | 1 MG | | | | | B-C | 0.824 | 0.824 | N/A |
| | | | | | | B-D | 0.504 | 0.504 | N/A |
| | | | | | | B-E | 0.180 | 0.070 | N/A |
| | | | | | | C-D | 0.822 | 0.822 | N/A |
| | | | | | | C-E | 0.075 | 0.024* | N/A |
| | | | | | | D-E | 0.026* | 0.007** | N/A |
| SOMNOLENCE | A) PLACEBO | 40 | 0 (0.0%) | 0 (0.0%) | 0 (0.0%) | TREATMENT | 0.009** | 0.009** | N/A |
| | B) MS 60 MG | 41 | 4 (9.8%) | 4 (9.8%) | 0 (0.0%) | A-B | 0.115 | 0.115 | N/A |
| | C) MS 60 MG/NTX | 41 | 1 (2.4%) | 0 (2.4%) | 0 (0.0%) | A-C | 1.000 | 1.000 | N/A |
| | 0.01 MG | | | | | | | | |
| | D) MS 60 MG/NTX | 41 | 3 (7.3%) | 3 (7.3%) | 0 (0.0%) | A-D | 0.240 | 0.240 | N/A |
| | 0.1 MG | | | | | | | | |
| | E) MS 60 MG/NTX | 41 | 8 (19.5%) | 8 (19.5%) | 0 (0.0%) | A-E | 0.005** | 0.005** | N/A |
| | 1 MG | | | | | B-C | 0.359 | 0.359 | N/A |
| | | | | | | B-D | 1.000 | 1.000 | N/A |
| | | | | | | B-E | 0.349 | 0.349 | N/A |
| | | | | | | C-D | 0.615 | 0.615 | N/A |
| | | | | | | C-E | 0.029* | 0.029* | N/A |
| | | | | | | D-E | 0.193 | 0.193 | N/A |
| VOMITING | A) PLACEBO | 40 | 3 (7.5%) | 3 (7.5%) | 0 (0.0%) | TREATMENT | <0.001*** | <0.001*** | N/A |
| | B) MS 60 MG | 41 | 18 (43.9%) | 18 (43.9%) | 0 (0.0%) | A-B | <0.001*** | <0.001*** | N/A |
| | C) MS 60 MG/NTX | 41 | 20 (48.8%) | 20 (48.8%) | 0 (0.0%) | A-C | <0.001** | <0.001*** | N/A |
| | 0.01 MG | | | | | | | | |
| | D) MS 60 MG/NTX | 41 | 19 (46.3%) | 19 (46.3%) | 0 (0.0%) | A-D | <0.001*** | <0.001** | N/A |
| | 0.1 MG | | | | | | | | |
| | E) MS 60 MG/NTX | 41 | 9 (22.0%) | 9 (22.0%) | 0 (0.0%) | A-E | 0.115 | 0.115 | N/A |
| | 1 MG | | | | | B-C | 0.824 | 0.824 | N/A |
| | | | | | | B-D | 1.000 | 1.000 | N/A |
| | | | | | | B-E | 0.059 | 0.059 | N/A |
| | | | | | | C-D | 1.000 | 1.000 | N/A |
| | | | | | | C-E | 0.020* | 0.020* | N/A |
| | | | | | | D-E | 0.035* | 0.035* | N/A |

[1] P-VALUE COMPARES THE PROPORTION OF SUBJECTS WITH EVENTS.
[2] RELATIONSHIP TO STUDY DRUG = ‘SUSPECTED’ OR ‘PROBABLE’.
N/A: NOT APPLICABLE.
*, **, ***P-VALUE <=0.05, <=0.01, OR <=0.001 RESPECTIVELY.

Although the invention herein has been described with reference to particular embodiments, it is to be understood that these embodiments are merely illustrative of various aspects of the invention. Thus, it is to be understood that numerous modifications may be made in the illustrative embodiments and other arrangements may be devised without departing from the spirit and scope of the invention.

### EXAMPLE 2

The results from the clinical study as described in Example 1 were analyzed by gender.

The results for females and males from the Example 1 clinical study are shown in the following Tables and Figures.

A total of 204 subjects were randomized; among them 201 subjects were deemed evaluable. One subject in each of the placebo, MS and MS/0.1 NTX groups was not evaluable because the subject took rescue medication less than 90 minutes after dosing. Tables 14A and 14B show the number of female and male subjects separately.

TABLE 14A
Analysis Populations, Female Patients
Treatments

| | | | MS (60 mg) | MS (60 mg) | MS (60 mg) | |
|---|---|---|---|---|---|---|
| | Placebo with | MS (60 mg) | with NTX | with NTX | with NTX | |
| | Placebo | with Placebo | (0.01 mg) | (0.1 mg) | (1.0 mg) | Total |
| Patients Enrolled [1] | 22 | 23 | 20 | 20 | 20 | 105 |
| Safety | 22 (100.0%) | 23 (100.0%) | 20 (100.0%) | 20 (100.0%) | 20 (100.0%) | 105 (100.0%) |
| Intent-To-Treat | 22 (100.0%) | 23 (100.0%) | 20 (100.0%) | 20 (100.0%) | 20 (100.0%) | 105 (100.0%) |
| Evaluable | 22 (100.0%) | 23 (100.0%) | 20 (100.0%) | 19 (95.0%) | 20 (100.0%) | 104 (99.0%) |

[1] PATIENTS WITH DEMOGRAPHIC INFORMATION.

TABLE 14B
Analysis Populations, Male Patients
Treatments

| | | | MS (60 mg) | MS (60 mg) | MS (60 mg) | |
|---|---|---|---|---|---|---|
| | Placebo with | MS (60 mg) | with NTX | with NTX | with NTX | |
| | Placebo | with Placebo | (0.01 mg) | (0.1 mg) | (1.0 mg) | Total |
| Patients Enrolled [1] | 18 | 18 | 21 | 21 | 21 | 99 |
| Safety | 18 (100.0%) | 18 (100.0%) | 21 (100.0%) | 21 (100.0%) | 21 (100.0%) | 99 (100.0%) |
| Intent-To-Treat | 18 (100.0%) | 18 (100.0%) | 21 (100.0%) | 21 (100.0%) | 21 (100.0%) | 99 (100.0%) |
| Evaluable | 17 (94.4%) | 17 (94.4%) | 21 (100.0%) | 21 (100.0%) | 21 (100.0%) | 97 (98.0%) |

[1] PATIENTS WITH DEMOGRAPHIC INFORMATION.

The demographic and baseline characteristics were summarized by treatment groups for the ITT population (all randomized patients) and the evaluable population (all randomized patients with at least one efficacy evaluation at 90 minutes or more after dosing) (Table 15A for females and Table 15B for males). Demographic characteristics included age, race/ethnicity, sex, weight, height, medical history, teeth extracted (impacted and non-impacted), baseline pain intensity, and baseline visual analog scale.

The demographics for the total ITT population were comparable across all 5 treatment groups. Female subjects (51%) ranged in age from 16 to 35 years; male subjects ranged in age from 16 to 39 years. There were some differences among treatment groups in the maximum degree of impaction of third molar extracted. No adjustments in the analyses were made to take into account these differences among treatment groups. Generally, no differences among overall treatment groups were noted in the number of patients with either a significant medical history or disease of any body system. The baseline pain intensity scores and visual analog scale scores, respectively, are shown in Tables 16A and 16B for females and Tables 16C and 16D for males.

TABLE 15A
Baseline Characteristics
Intent-To-Treat Population, Female Patients

| | | | | MS (60 mg) | | | |
|---|---|---|---|---|---|---|---|
| | | | | with NTX | MS (60 mg) with | MS (60 mg) with | |
| | | Placebo | MS (60 mg) | (0.01 mg) | NTX (0.1 mg) | NTX 1.0 mg) | P-Value |
| Age (yrs) | N | 22 | 23 | 20 | 20 | 20 | 0.294 [1] |
| | Mean | 21.6 | 22.6 | 21.4 | 23.5 | 22.9 | |
| | SD | 2.63 | 3.92 | 2.56 | 5.03 | 3.18 | |
| | Median | 21.0 | 22.0 | 21.0 | 22.0 | 23.0 | |
| | Range | 19-27 | 19-32 | 18-28 | 16-35 | 19-29 | |
| Race/Ethnic | Caucasian | 13 (59.1%) | 12 (52.2%) | 15 (75.0%) | 12 (60.0%) | 14 (70.0%) | 0.566 [2] |
| Origin (N, %) [3] | Black | 4 (18.2%) | 2 (8.7%) | 1 (5.0%) | 1 (5.0%) | 1 (5.0%) | |
| | Asian | 2 (9.1%) | 1 (4.3%) | 0 (0.0%) | 2 (10.0%) | 1 (5.0%) | |
| | Hispanic | 3 (13.6%) | 8 (34.8%) | 4 (20.0%) | 5 (25.0%) | 4 (20.0%) | |
| | Total | 22 | 23 | 20 | 20 | 20 | |
| Height (cm) | N | 22 | 23 | 20 | 20 | 20 | 0.323 [1] |
| | Mean | 165.0 | 163.1 | 167.2 | 163.5 | 163.6 | |
| | SD | 7.48 | 6.96 | 5.42 | 8.52 | 6.48 | |
| | Median | 165.1 | 162.6 | 167.6 | 163.2 | 162.6 | |
| | Range | 152.4-179.1 | 149.9-177.8 | 157.5-176.5 | 139.7-177.8 | 154.9-180.3 | |
| Weight (kg) | N | 22 | 23 | 20 | 20 | 20 | 0.535 [1] |
| | Mean | 64.5 | 68.1 | 67.5 | 61.4 | 67.3 | |
| | SD | 14.00 | 15.87 | 13.55 | 9.37 | 17.98 | |
| | Median | 60.5 | 65.0 | 66.2 | 61.8 | 62.1 | |
| | Range | 47.3-106.4 | 42.7-117.3 | 50.9-105.5 | 46.4-79.1 | 47.3-105.9 | |
| Number of | 2 | 22 (100.0%) | 23 (100.0%) | 20 (100.0%) | 19 (95.0%) | 19 (95.0%) | 0.324 [2] |
| Third Molars | 3 | 0 (0.0%) | 0 (0.0%) | 0 (0.0%) | 0 (0.0%) | 1 (5.0%) | |
| Extracted | 4 | 0 (0.0%) | 0 (0.0%) | 0 (0.0%) | 1 (5.0%) | 0 (0.0%) | |
| (N, %) [4] | TOTAL | 22 | 23 | 20 | 20 | 20 | |
| Time | N | 22 | 23 | 20 | 20 | 20 | 0.741 [2] |
| Between End | Mean | 137.8 | 144.9 | 145.6 | 156.3 | 141.5 | |
| of Surgery | SD | 36.86 | 47.22 | 54.74 | 47.28 | 33.94 | |
| and Study | Median | 130.0 | 138.0 | 134.5 | 156.5 | 146.0 | |
| Medication | Range | 79.0-222.0 | 71.0-259.0 | 88.0-299.0 | 78.0-255.0 | 88.0-224.0 | |
| (Minutes) | | | | | | | |

[1] One-Way Analysis of Variance with Treatment as the Factor.
[2] Fisher's Exact Test.
[3] Black, Asian, Hispanic, and Other are Combined into One Category to Derive P-Value.
[4] 3 or More Third Molars Extracted as One Category to Derive P-Value.

TABLE 15B
Baseline Characteristics
Intent-To-Treat Population, Male Patients

| | | | | MS (60 mg) | | | |
|---|---|---|---|---|---|---|---|
| | | | | with NTX | MS (60 mg) with | MS (60 mg) with | |
| | | Placebo | MS (60 mg) | (0.01 mg) | NTX (0.1 mg) | NTX 1.0 mg) | P-Value |
| Age (yrs) | N | 18 | 18 | 21 | 21 | 21 | 0.980 [1] |
| | Mean | 22.6 | 23.1 | 22.7 | 22.7 | 22.1 | |
| | SD | 3.24 | 3.90 | 4.24 | 5.25 | 5.17 | |
| | Median | 22.0 | 22.5 | 21.0 | 21.0 | 20.0 | |
| | Range | 18-28 | 19-31 | 18-35 | 16-39 | 18-39 | |
| Race | Caucasian | 13 (72.2%) | 13 (72.2%) | 16 (76.2%) | 16 (76.2%) | 12 (57.1%) | 0.688 [2] |
| | Black | 0 (0.0%) | 2 (11.1%) | 0 (0.0%) | 0 (0.0%) | 2 (9.5%) | |
| | Asian | 1 (5.6%) | 0 (0.0%) | 1 (4.8%) | 0 (0.0%) | 4 (19.0%) | |
| | Hispanic | 4 (22.2%) | 3 (16.7%) | 3 (14.3%) | 4 (19.0%) | 2 (9.5%) | |
| | Other | 0 (0.0%) | 0 (0.0%) | 1 (4.8%) | 1 (4.8%) | 1 (4.8%) | |
| | Total | 18 | 18 | 21 | 21 | 21 | |
| Height (cm) | N | 18 | 18 | 21 | 21 | 21 | 0.666 [1] |
| | Mean | 176.8 | 180.4 | 180.2 | 179.0 | 178.8 | |
| | SD | 8.13 | 10.47 | 7.87 | 6.62 | 6.68 | |
| | Median | 177.8 | 180.3 | 182.9 | 180.3 | 177.8 | |
| | Range | 160.0-188.0 | 152.9-198.1 | 162.6-193.0 | 167.6-194.3 | 165.1-188.0 | |
| Weight (kg) | N | 18 | 18 | 21 | 21 | 21 | 0.145 [1] |
| | Mean | 74.1 | 85.0 | 76.5 | 79.8 | 77.6 | |
| | SD | 12.24 | 14.70 | 11.03 | 12.72 | 15.47 | |
| | Median | 72.5 | 81.8 | 77.7 | 75.5 | 73.6 | |
| | Range | 56.8-103.6 | 64.1-114.5 | 55.9-95.5 | 56.8-104.5 | 56.8-122.3 | |
| Number of | 2 | 18 (100.0%) | 17 (94.4%) | 21 (100.0%) | 18 (85.7%) | 21 (100.0%) | 0.275 [2] |
| Third Molars | 3 | 0 (0.0%) | 1 (5.6%) | 0 (0.0%) | 2 (9.5%) | 0 (0.0%) | |
| Extracted | 4 | 0 (0.0%) | 0 (0.0%) | 0 (0.0%) | 1 (4.8%) | 0 (0.0%) | |
| (N, %) [4] | TOTAL | 18 | 18 | 21 | 21 | 21 | |
| Time | N | 18 | 18 | 21 | 21 | 21 | 0.797 [2] |
| Between End | Mean | 169.8 | 150.4 | 156.4 | 156.6 | 152.1 | |
| of Surgery | SD | 55.51 | 34.90 | 40.98 | 64.90 | 50.28 | |
| and Study | Median | 159.0 | 151.0 | 155.0 | 152.0 | 149.0 | |
| Medication | Range | 92.0-307.0 | 88.0-216.0 | 82.0-226.0 | 62.0-303.0 | 76.0-277.0 | |
| (Minutes) | | | | | | | |

[1] One-Way Analysis of Variance with Treatment as the Factor.
[2] Fisher's Exact Test.
[3] Black, Asian, Hispanic, and Other are Combined into One Category to Derive P-Value.
[4] 3 or More Third Molars Extracted as One Category to Derive P-Value.

TABLE 16A
Baseline Pain Intensity Scores
Intent-To-Treat Population, Female Patients

| | | | P-VALUE FOR PAIRWISE COMPARISONS | | | | |
|---|---|---|---|---|---|---|---|
| | | | | MS 60 mg | MS 60 mg | MS 60 mg | P-VALUE FOR |
| | PAIN INTENSITY | | MS | NTX | NTX | NTX | OVERALL |
| TREATMENT | MODERATE | SEVERE | 60 mg | 0.01 mg | 0.1 mg | 1 mg | TREATMENT |
| Placebo | 6 (27.3%) | 16 (72.7%) | 0.749 | 0.515 | 0.335 | 0.335 | 0.722 |
| MS 60 mg | 8 (34.8%) | 15 (65.2%) | | 0.761 | 0.545 | 0.545 | |
| MS 60 mg/NTX 0.01 mg | 8 (40.0%) | 12 (60.0%) | | | 1.000 | 1.000 | |
| MS 60 mg/NTX 0.1 mg | 9 (45.0%) | 11 (55.0%) | | | | 1.000 | |
| MS 60 mg/NTX 1 mg | 9 (45.0%) | 11 (55.0%) | | | | | |

NOTE:
P-VALUES ARE FROM FISHER'S EXACT TEST.

TABLE 16B
Baseline Visual Analog Scale (VAS) Scores
Intent-To-Treat Population, Female Patients

| | | | | | | | | | | P-VALUE FOR PAIRWISE COMPARISONS | | | | |
|---|---|---|---|---|---|---|---|---|---|---|---|---|---|---|
| | | | | | | | | | | | | | | P-Value |
| | BASELINE VAS SCORE | | | | | | | | | | MS 60 | MS 60 | | for |
| | Moderate [1] | | | Severe [1] | | | Total | | | MS | mg NTX | mg NTX | MS 60 mg | Overall |
| TREATMENT | N | Mean | (SD) | N | Mean | (SD) | N | Mean | (SD) | 60 mg | 0.01 mg | 0.1 mg | NTX 1 mg | Treatment |
| Placebo | 6 | 65.0 | (8.02) | 16 | 80.0 | (11.33) | 22 | 75.9 | (12.40) | 0.256 | 0.300 | 0.452 | 0.776 | 0.257 |
| MS 60 mg | 8 | 68.4 | (7.67) | 15 | 87.0 | (6.80) | 23 | 80.5 | (11.42) | | 0.032 | 0.736 | 0.410 | |
| MS 60 mg/ | 8 | 59.0 | (8.50) | 12 | 79.9 | (13.15) | 20 | 71.6 | (15.40) | | | 0.084 | 0.198 | |
| NTX 0.01 mg | | | | | | | | | | | | | | |
| MS 60 mg/ | 8 | 67.9 | (14.61) | 11 | 87.3 | (9.22) | 19 | 79.1 | (15.07) | | | | 0.644 | |
| NTX 0.1 mg | | | | | | | | | | | | | | |
| MS 60 mg/ | 9 | 69.9 | (12.19) | 11 | 83.0 | (11.93) | 20 | 77.1 | (13.50) | | | | | |
| NTX 1 mg | | | | | | | | | | | | | | |

NOTE:
P-VALUES ARE FROM ONE-WAY ANALYSIS OF VARIANCE AND ITS CONTRASTS
[1] BASELINE PAIN INTENSITY ON THE CATEGORICAL SCALE.

TABLE 16C
Baseline Pain Intensity Scores
Intent-To-Treat Population, Male Patients

| | | | P-VALUE FOR PAIRWISE COMPARISONS | | | | |
|---|---|---|---|---|---|---|---|
| | | | | MS 60 mg | MS 60 mg | MS 60 mg | P-VALUE FOR |
| | PAIN INTENSITY | | MS | NTX | NTX | NTX | OVERALL |
| TREATMENT | MODERATE | SEVERE | 60 mg | 0.01 mg | 0.1 mg | 1 mg | TREATMENT |
| Placebo | 10 (55.6%) | 8 (44.4%) | 1.000 | 0.527 | 0.527 | 0.343 | 0.749 |
| MS 60 mg | 10 (55.6%) | 8 (44.4%) | | 0.527 | 0.527 | 0.343 | |
| MS 60 mg/NTX 0.01 mg | 9 (42.9%) | 12 (57.1%) | | | 1.000 | 1.000 | |
| MS 60 mg/NTX 0.1 mg | 9 (42.9%) | 12 (57.1%) | | | | 1.000 | |
| MS 60 mg/NTX 1 mg | 8 (38.1%) | 13 (61.9%) | | | | | |

NOTE:
P-VALUES ARE FROM FISHER'S EXACT TEST.

TABLE 16D
Baseline Visual Analog Scale (VAS) Scores
Intent-To-Treat Population, Male Patients

| | | | | | | | | | | P-VALUE FOR PAIRWISE COMPARISONS | | | | |
|---|---|---|---|---|---|---|---|---|---|---|---|---|---|---|
| | | | | | | | | | | | | | | P-Value |
| | BASELINE VAS SCORE | | | | | | | | | | MS 60 | | | for |
| | Moderate [1] | | | Severe [1] | | | Total | | | MS | mg NTX | MS 60 mg | MS 60 mg | Overall |
| TREATMENT | N | Mean | (SD) | N | Mean | (SD) | N | Mean | (SD) | 60 mg | 0.01 mg | NTX 0.1 mg | NTX 1 mg | Treatment |
| Placebo | 10 | 65.8 | (8.26) | 8 | 78.3 | (6.76) | 18 | 71.3 | (9.77) | 0.719 | 0.271 | 0.346 | 0.821 | 0.586 |
| MS 60 mg | 10 | 67.8 | (6.00) | 8 | 78.8 | (8.35) | 18 | 72.7 | (8.89) | | 0.465 | 0.568 | 0.550 | |
| MS 60 mg/NTX 0.01 mg | 9 | 62.2 | (10.20) | 12 | 85.1 | (7.40) | 21 | 75.3 | (14.36) | | | 0.868 | 0.168 | |
| MS 60 mg/NTX 0.1 mg | 9 | 63.3 | (5.29) | 12 | 83.3 | (9.11) | 21 | 74.7 | (12.60) | | | | 0.225 | |
| MS 60 mg/NTX 1 mg | 8 | 65.0 | (8.32) | 13 | 73.9 | (6.40) | 21 | 70.5 | (8.27) | | | | | |

NOTE:
P-VALUES ARE FROM ONE-WAY ANALYSIS OF VARIANCE AND ITS CONTRASTS
[1] BASELINE PAIN INTENSITY ON THE CATEGORICAL SCALE.

The TOTPAR results (4 hour, 6 hour, 8 hour) are summarized in Tables 17A for females and 17B for males. The placebo treatment group had the lowest mean TOTPAR scores. All 4 of the active treatment groups exhibited mean TOTPAR scores that were numerically higher than placebo. In females, the mean TOTPAR scores for the 0.01 mg NTX and 0.1 mg NTX combination treatments were higher than that for the MS alone treatment, whereas the 1.0 mg NTX combination treatment mean was comparable to or lower than that for the MS alone. In males, the scores for the 1.0 mg NTX, 0.1 mg NTX, and 0.01 mg combination treatments were higher than that for the MS alone treatment for 4 hour and 6 hour TOTPAR scores, and the 1.0 mg and 0.01 mg NTX combinations were higher than morphine alone for the 8 hour TOTPAR scores.

TABLE 17A
Total Pain Relief Scores
Intent-To-Treat Population, Female Patients

| TOTAL PAIN RELIEF SCORE | | | | | | | | |
|---|---|---|---|---|---|---|---|---|
| TREATMENT | N | MEAN | SD | MIN | MEDIAN | MAX | SOURCE | P-VALUE [1] |

TOTAL PAIN RELIEF SCORE (0-4 HOURS)

| | | | | | | | | |
|---|---|---|---|---|---|---|---|---|
| A) Placebo | 22 | 1.86 | 2.677 | 0.0 | 0.00 | 8.0 | TRT | <0.001** |
| B) MS 60 mg | 23 | 5.07 | 4.478 | 0.0 | 5.75 | 13.2 | B-A | 0.006** |
| C) MS 60 mg/NTX 0.01 mg | 20 | 6.18 | 3.948 | 0.0 | 5.99 | 14.0 | C-A | <0.001*** |
| D) MS 60 mg/NTX 0.1 mg | 20 | 6.00 | 4.208 | 0.0 | 6.74 | 12.0 | D-A | <0.001*** |
| E) MS 60 mg/NTX 1 mg | 20 | 3.14 | 3.928 | 0.0 | 1.00 | 11.3 | E-A | 0.290 |
| | | | | | | | C-B | 0.352 |
| | | | | | | | D-B | 0.432 |
| | | | | | | | E-B | 0.109 |

TOTAL PAIN RELIEF SCORE (0-6 HOURS)

| | | | | | | | | |
|---|---|---|---|---|---|---|---|---|
| A) Placebo | 22 | 3.16 | 4.635 | 0.0 | 0.00 | 14.0 | TRT | <0.001** |
| B) MS 60 mg | 23 | 8.38 | 7.548 | 0.0 | 11.25 | 21.2 | B-A | 0.007** |
| C) MS 60 mg/NTX 0.01 mg | 20 | 9.63 | 6.172 | 0.0 | 9.60 | 20.5 | C-A | <0.001** |
| D) MS 60 mg/NTX 0.1 mg | 20 | 9.76 | 7.172 | 0.0 | 10.75 | 20.0 | D-A | <0.001** |
| E) MS 60 mg/NTX 1 mg | 20 | 4.59 | 6.202 | 0.0 | 1.00 | 16.5 | E-A | 0.473 |
| | | | | | | | C-B | 0.527 |
| | | | | | | | D-B | 0.484 |
| | | | | | | | E-B | 0.056 |

TOTAL PAIN RELIEF SCORE (0-8 HOURS)

| | | | | | | | | |
|---|---|---|---|---|---|---|---|---|
| A) Placebo | 22 | 4.45 | 6.666 | 0.0 | 0.00 | 20.5 | TRT | 0.002** |
| B) MS 60 mg | 23 | 11.68 | 10.691 | 0.0 | 16.48 | 29.2 | B-A | 0.009** |
| C) MS 60 mg/NTX 0.01 mg | 20 | 12.97 | 8.787 | 0.0 | 11.25 | 27.0 | C-A | 0.003** |
| D) MS 60 mg/NTX 0.1 mg | 20 | 13.66 | 10.500 | 0.0 | 15.75 | 28.0 | D-A | <0.001** |
| E) MS 60 mg/NTX 1 mg | 20 | 6.19 | 8.905 | 0.0 | 1.00 | 24.5 | E-A | 0.544 |
| | | | | | | | C-B | 0.650 |
| | | | | | | | D-B | 0.485 |
| | | | | | | | E-B | 0.054 |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.

TABLE 17B
Total Pain Relief Scores
Intent-To-Treat Population, Male Patients

| TOTAL PAIN RELIEF SCORE | | | | | | | | |
|---|---|---|---|---|---|---|---|---|
| TREATMENT | N | MEAN | SD | MIN | MEDIAN | MAX | SOURCE | P-VALUE [1] |

TOTAL PAIN RELIEF SCORE (0-4 HOURS)

| | | | | | | | | |
|---|---|---|---|---|---|---|---|---|
| A) Placebo | 18 | 2.61 | 3.044 | 0.0 | 1.50 | 9.5 | TRT | 0.281 |
| B) MS 60 mg | 18 | 3.49 | 3.301 | 0.0 | 3.50 | 10.1 | B-A | N/D |
| C) MS 60 mg/NTX 0.01 mg | 21 | 4.85 | 4.243 | 0.0 | 5.73 | 14.0 | C-A | N/D |
| D) MS 60 mg/NTX 0.1 mg | 21 | 4.22 | 4.261 | 0.0 | 3.00 | 12.3 | D-A | N/D |
| E) MS 60 mg/NTX 1 mg | 21 | 5.18 | 4.757 | 0.0 | 5.25 | 14.0 | E-A | N/D |
| | | | | | | | C-B | N/D |
| | | | | | | | D-B | N/D |
| | | | | | | | E-B | N/D |

TOTAL PAIN RELIEF SCORE (0-6 HOURS)

| | | | | | | | | |
|---|---|---|---|---|---|---|---|---|
| A) Placebo | 18 | 4.19 | 5.179 | 0.0 | 1.50 | 14.5 | TRT | 0.299 |
| B) MS 60 mg | 18 | 6.41 | 6.165 | 0.0 | 6.75 | 18.1 | B-A | N/D |
| C) MS 60 mg/NTX 0.01 mg | 21 | 8.11 | 6.810 | 0.0 | 9.23 | 20.0 | C-A | N/D |
| D) MS 60 mg/NTX 0.1 mg | 21 | 6.82 | 6.872 | 0.0 | 5.25 | 20.3 | D-A | N/D |
| E) MS 60 mg/NTX 1 mg | 21 | 8.51 | 7.841 | 0.0 | 8.75 | 22.0 | E-A | N/D |
| | | | | | | | C-B | N/D |
| | | | | | | | D-B | N/D |
| | | | | | | | E-B | N/D |

TOTAL PAIN RELIEF SCORE (0-8 HOURS)

| | | | | | | | | |
|---|---|---|---|---|---|---|---|---|
| A) Placebo | 18 | 5.94 | 7.553 | 0.0 | 1.50 | 20.0 | TRT | 0.334 |
| B) MS 60 mg | 18 | 9.52 | 9.168 | 0.0 | 10.38 | 26.1 | B-A | N/D |
| C) MS 60 mg/NTX 0.01 mg | 21 | 11.38 | 9.611 | 0.0 | 13.73 | 27.5 | C-A | N/D |
| D) MS 60 mg/NTX 0.1 mg | 21 | 9.48 | 9.569 | 0.0 | 7.25 | 28.3 | D-A | N/D |
| E) MS 60 mg/NTX 1 mg | 21 | 11.94 | 11.02 | 0.0 | 11.26 | 30.0 | E-A | N/D |
| | | | | | | | C-B | N/D |
| | | | | | | | D-B | N/D |
| | | | | | | | E-B | N/D |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.

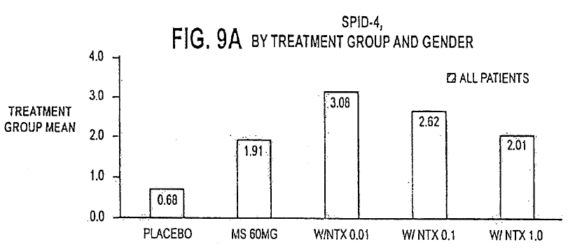

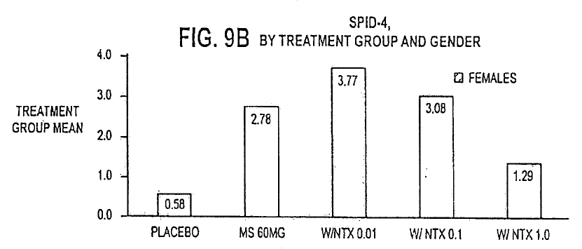

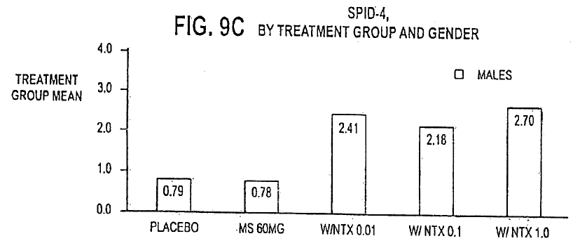

Tables 18A for females and 18B for males, summarize the results of the 4, 6, and 8 hour SPID results and the 4 hour SPID results are shown in FIGS. 9B for females and 9C for males. In females, the placebo treatment had the lowest mean 4, 6 and 8 hour SPID scores. All 4 of the active treatment groups exhibited improved profiles in mean SPID relative to placebo. The mean SPID scores for the 0.01 mg NTX and 0.1 mg NTX combination treatments were higher than that for the MS alone treatment. In males, the placebo treatment had the lowest mean 6 and 8 hour SPID scores. For the 4 hour SPID score, the placebo treatment was similar to the MS alone treatment. The mean SPID scores for the 0.01 mg NTX, 0.1 mg NTX and 1.0 mg combination treatments were higher than that for the MS alone treatment.

TABLE 18A
Sum of Pain Intensity Differences
Intent-To-Treat Population, Female Patients

| SUM OF PAIN INTENSITY DIFFERENCES [1] | | | | | | | | |
|---|---|---|---|---|---|---|---|---|
| TREATMENT | N | MEAN | SD | MIN | MEDIAN | MAX | SOURCE | P-VALUE [2] |

SUM OF PAIN INTENSITY DIFFERENCES (0-4 HOURS)

| | | | | | | | | |
|---|---|---|---|---|---|---|---|---|
| A) Placebo | 22 | 0.58 | 2.047 | −3.8 | 0.00 | 4.5 | TRT | 0.002** |
| B) MS 60 mg | 23 | 2.78 | 3.429 | −3.3 | 2.50 | 8.0 | B-A | 0.012* |
| C) MS 60 mg/NTX 0.01 mg | 20 | 3.77 | 2.727 | 0.0 | 3.12 | 10.3 | C-A | <0.001*** |
| D) MS 60 mg/NTX 0.1 mg | 20 | 3.08 | 2.663 | 0.0 | 2.36 | 7.5 | D-A | 0.006** |
| E) MS 60 mg/NTX 1 mg | 20 | 1.29 | 3.434 | −3.8 | 0.00 | 7.5 | E-A | 0.433 |
| | | | | | | | C-B | 0.268 |
| | | | | | | | D-B | 0.743 |
| | | | | | | | E-B | 0.095 |

SUM OF PAIN INTENSITY DIFFERENCES (0-6 HOURS)

| | | | | | | | | |
|---|---|---|---|---|---|---|---|---|
| A) Placebo | 22 | 1.10 | 3.350 | −5.8 | 0.00 | 8.3 | TRT | 0.002** |
| B) MS 60 mg | 23 | 4.56 | 5.676 | −5.3 | 4.50 | 12.0 | B-A | 0.015* |
| C) MS 60 mg/NTX 0.01 mg | 20 | 5.90 | 4.227 | 0.0 | 6.23 | 15.3 | C-A | <0.001** |
| D) MS 60 mg/NTX 0.1 mg | 20 | 5.22 | 4.382 | 0.0 | 5.12 | 11.5 | D-A | 0.005** |
| E) MS 60 mg/NTX 1 mg | 20 | 1.82 | 5.388 | −5.8 | 0.00 | 12.0 | E-A | 0.619 |
| | | | | | | | C-B | 0.351 |
| | | | | | | | D-B | 0.645 |
| | | | | | | | E-B | 0.059 |

SUM OF PAIN INTENSITY DIFFERENCES (0-8 HOURS)

| | | | | | | | | |
|---|---|---|---|---|---|---|---|---|
| A) Placebo | 22 | 1.58 | 4.741 | −7.8 | 0.00 | 12.8 | TRT | 0.004** |
| B) MS 60 mg | 23 | 6.34 | 8.005 | −7.3 | 6.50 | 17.3 | B-A | 0.018* |
| C) MS 60 mg/NTX 0.01 mg | 20 | 7.86 | 6.023 | 0.0 | 8.37 | 19.8 | C-A | 0.003** |
| D) MS 60 mg/NTX 0.1 mg | 20 | 7.52 | 6.389 | 0.0 | 7.63 | 16.8 | D-A | 0.004** |
| E) MS 60 mg/NTX 1 mg | 20 | 2.52 | 7.710 | −7.8 | 0.00 | 18.0 | E-A | 0.648 |
| | | | | | | | C-B | 0.458 |
| | | | | | | | D-B | 0.565 |
| | | | | | | | E-B | 0.065 |

[1] PAIN INTENSITY DIFFERENCE = PAIN INTENSITY AT BASELINE − PAIN INTENSITY AT CURRENT TIME.
[2] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.

TABLE 18B
Sum of Pain Intensity Differences
Intent-To-Treat Population, Male Patients

| SUM OF PAIN INTENSITY DIFFERENCES [1] | | | | | | | | |
|---|---|---|---|---|---|---|---|---|
| TREATMENT | N | MEAN | SD | MIN | MEDIAN | MAX | SOURCE | P-VALUE [2] |

SUM OF PAIN INTENSITY DIFFERENCES (0-4 HOURS)

| | | | | | | | | |
|---|---|---|---|---|---|---|---|---|
| A) Placebo | 18 | 0.79 | 2.356 | −3.8 | 0.25 | 5.0 | TRT | 0.200 |
| B) MS 60 mg | 18 | 0.78 | 2.823 | −3.8 | 1.88 | 4.0 | B-A | N/D |
| C) MS 60 mg/NTX 0.01 mg | 21 | 2.41 | 3.726 | −3.8 | 3.25 | 10.3 | C-A | N/D |
| D) MS 60 mg/NTX 0.1 mg | 21 | 2.18 | 2.901 | −3.8 | 2.49 | 8.5 | D-A | N/D |
| E) MS 60 mg/NTX 1 mg | 21 | 2.70 | 4.011 | −3.8 | 3.74 | 8.5 | E-A | N/D |
| | | | | | | | C-B | N/D |
| | | | | | | | D-B | N/D |
| | | | | | | | E-B | N/D |

SUM OF PAIN INTENSITY DIFFERENCES (0-6 HOURS)

| | | | | | | | | |
|---|---|---|---|---|---|---|---|---|
| A) Placebo | 18 | 1.21 | 3.633 | −5.8 | 0.25 | 7.5 | TRT | 0.245 |
| B) MS 60 mg | 18 | 1.75 | 5.008 | −5.8 | 4.13 | 8.0 | B-A | N/D |
| C) MS 60 mg/NTX 0.01 mg | 21 | 3.86 | 5.683 | −5.8 | 5.00 | 14.3 | C-A | N/D |
| D) MS 60 mg/NTX 0.1 mg | 21 | 3.54 | 4.769 | −5.8 | 3.00 | 14.5 | D-A | N/D |
| E) MS 60 mg/NTX 1 mg | 21 | 4.51 | 6.634 | −5.8 | 5.74 | 14.5 | E-A | N/D |
| | | | | | | | C-B | N/D |
| | | | | | | | D-B | N/D |
| | | | | | | | E-B | N/D |

SUM OF PAIN INTENSITY DIFFERENCES (0-8 HOURS)

| | | | | | | | | |
|---|---|---|---|---|---|---|---|---|
| A) Placebo | 18 | 1.74 | 4.966 | −7.8 | 0.50 | 10.0 | TRT | 0.274 |
| B) MS 60 mg | 18 | 2.84 | 7.329 | −7.8 | 6.13 | 12.0 | B-A | N/D |
| C) MS 60 mg/NTX 0.01 mg | 21 | 5.45 | 7.943 | −7.8 | 6.00 | 19.8 | C-A | N/D |
| D) MS 60 mg/NTX 0.1 mg | 21 | 4.92 | 6.661 | −7.8 | 3.00 | 20.5 | D-A | N/D |
| E) MS 60 mg/NTX 1 mg | 21 | 6.47 | 9.353 | −7.8 | 7.74 | 20.0 | E-A | N/D |
| | | | | | | | C-B | N/D |
| | | | | | | | D-B | N/D |
| | | | | | | | E-B | N/D |

[1] PAIN INTENSITY DIFFERENCE = PAIN INTENSITY AT BASELINE − PAIN INTENSITY AT CURRENT TIME.
[2] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.

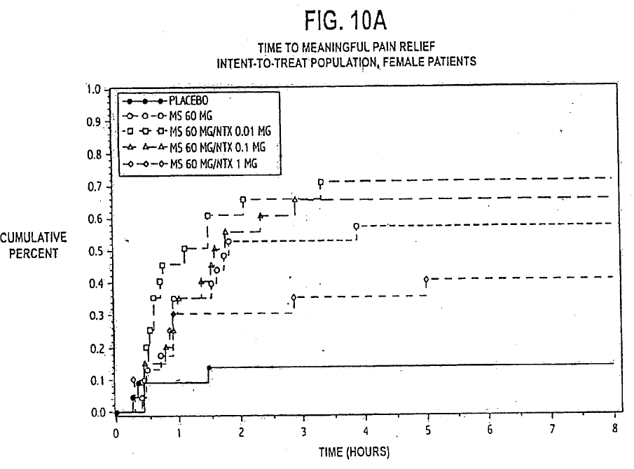

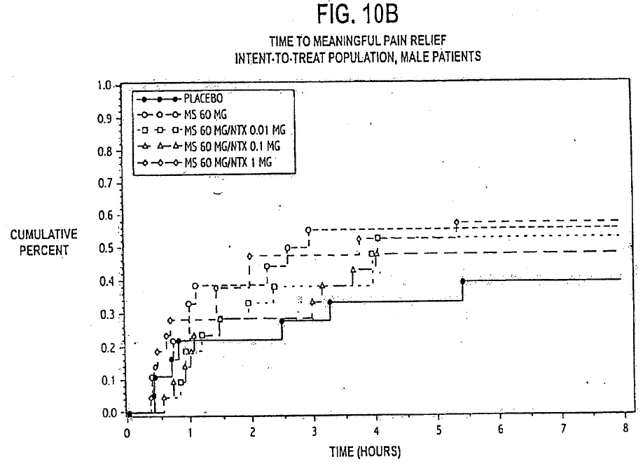

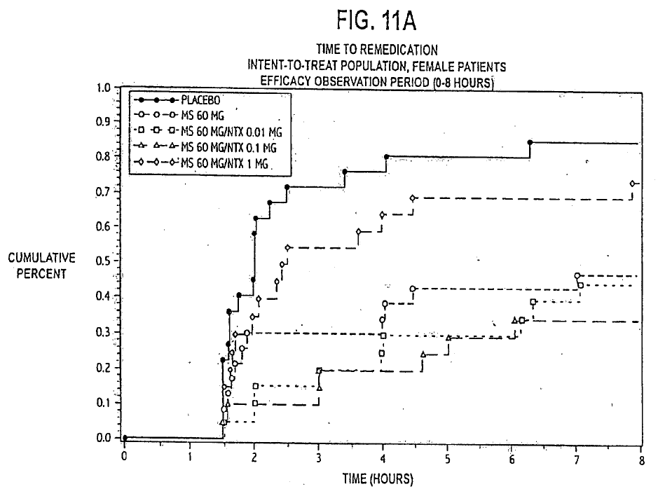

FIGS. 10A for females and 10B for males are visual presentations of the summary and analysis of time to onset of meaningful pain relief scores presented in Tables 19A for females and 19B for males. In females, the median time to onset of meaningful pain relief was shortest in the 0.01 mg NTX (low-dose) combination treatment group. In males, the median time to onset of meaningful pain relief was shortest for the MS alone treatment, followed by the 1.0 mg NTX combination and then the 0.01 mg NTX combination.

TABLE 19A
Time To Onset of Meaningful Pain Relief
Intent-To-Treat Population, Female Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 22 | >8:00 | (>8:00, >8:00) | TREATMENT | 0.004** | 0.013* |
| B) MS 60 mg | 23 | 1:50 | (0:57, >8:00) | B-A | 0.005** | 0.009** |
| C) MS 60 mg/NTX 0.01 mg | 20 | 1:18 | (0:37, >8:00) | C-A | <0.001*** | <0.001** |
| D) MS 60 mg/NTX 0.1 mg | 20 | 1:41 | (0:56, >8:00) | D-A | <0.001** | 0.003** |
| E) MS 60 mg/NTX 1 mg | 20 | >8:00 | (0:56, >8:00) | E-A | 0.064 | 0.077 |
| | | | | C-B | 0.254 | 0.212 |
| | | | | D-B | 0.591 | 0.642 |
| | | | | E-B | 0.385 | 0.538 |

*, **, ***P-VALUE <=0.05, <=0.01, or <= <0.001 RESPECTIVELY.
N/D: NOT DONE (BECAUSE OVERALL P-VALUE NOT SIGNIFICANT).

TABLE 19B
Time To Onset of Meaningful Pain Relief
Intent-To-Treat Population, Male Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 18 | >8:00 | (3:17, >8:00) | TREATMENT | 0.732 | 0.648 |
| B) MS 60 mg | 18 | 2:47 | (1:00, >8:00) | B-A | N/D | N/D |
| C) MS 60 mg/NTX 0.01 mg | 21 | 4:05 | (1:58, >8:00) | C-A | N/D | N/D |
| D) MS 60 mg/NTX 0.1 mg | 21 | >8:00 | (3:00, >8:00) | D-A | N/D | N/D |
| E) MS 60 mg/NTX 1 mg | 21 | 3:47 | (1:27, >8:00) | E-A | N/D | N/D |
| | | | | C-B | N/D | N/D |
| | | | | D-B | N/D | N/D |
| | | | | E-B | N/D | N/D |

*, **, ***P-VALUE <=0.05, <=0.01, or <= <0.001 RESPECTIVELY.
N/D: NOT DONE (BECAUSE OVERALL P-VALUE NOT SIGNIFICANT).

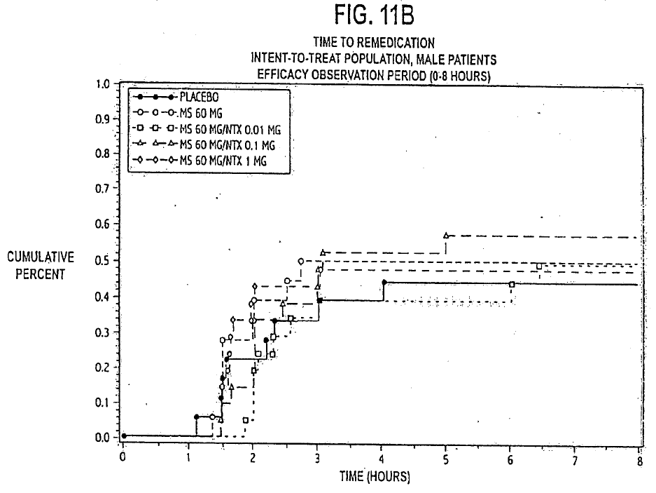

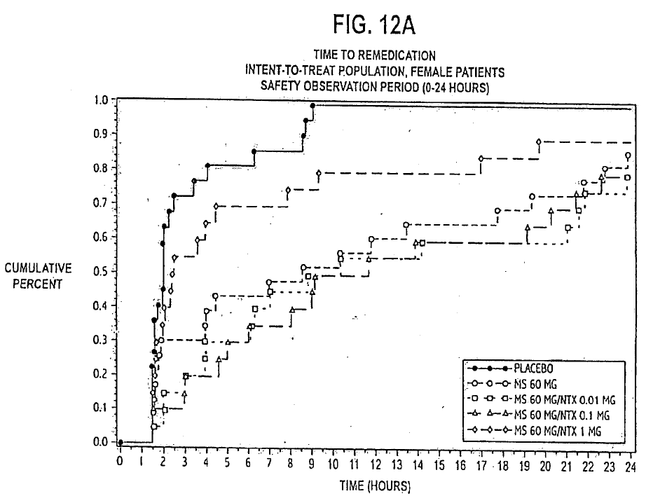

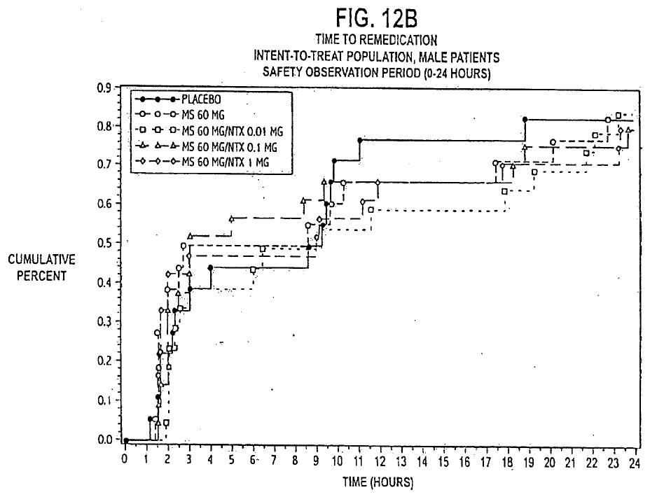

FIGS. 11A and 12A for females and 11B and 12B for males are visual presentations of the summary and analysis of time to remedication (rescue medication) up to 8 and 24 hours, respectively, presented in Tables 20A for females and 20B for males. The survival distributions (0-8 hours) were different across treatment groups (FIGS. 11A and 11B). In females, the survival distributions were different for the low-dose and mid-dose groups compared to placebo. The median times to administration of rescue medication were longer for the morphine (>8 hours), low-dose (>8 hours), and mid-dose (>8 hours) groups compared to the high-dose (2 hours, 30 minutes) and placebo (2 hours, 2 minutes) groups. In males, the median times to administration of rescue medication were longer for the placebo (>8 hours), MS alone (>8 hours), low-dose (>8 hours) and high-dose (>8 hours) compared to the mid-dose (3 hours, 6 minutes) group.

The survival distributions (0-24 hours) were also different across treatment groups (FIGS. 12A and 12B). In females, the median times to administration of rescue medication were longer for the morphine, low-dose, and mid-dose groups. In males, the median times to administration of rescue medication were longest for the low-dose and high-dose groups.

Analyses of time to remedication up to 24 hours yielded similar results, however, the data should be viewed with caution because subjects were not under close supervision after 8 hours.

TABLE 20A
Time To Rescue Medication
Intent-To-Treat Population, Female Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| EFFICACY OBSERVATION | | | | | | |
| PERIOD (0-8 HOURS) | | | | | | |
| A) Placebo | 22 | 2:02 | (1:38, 2:32) | Treatment | <0.001*** | <0.001*** |
| B) MS 60 mg | 23 | >8:00 | (4:01, >8:00) | B-A | 0.004** | 0.010* |
| C) MS 60 mg/NTX 0.01 mg | 20 | >8:00 | (4:02, >8:00) | C-A | <0.001*** | <0.001*** |
| D) MS 60 mg/NTX 0.1 mg | 20 | >8:00 | (5:03, >8:00) | D-A | <0.001*** | <0.001*** |
| E) MS 60 mg/NTX 1 mg | 20 | 2:30 | (1:44, 7:54) | E-A | 0.205 | 0.172 |
| | | | | C-B | 0.659 | 0.493 |
| | | | | D-B | 0.341 | 0.303 |
| | | | | E-B | 0.081 | 0.128 |
| SAFETY OBSERVATION | | | | | | |
| PERIOD (0-24 HOURS) | | | | | | |
| A) Placebo | 22 | 2:02 | (1:38, 2:32) | Treatment | <0.001*** | <0.001*** |
| B) MS 60 mg | 23 | 8:37 | (4:01, 17:45) | B-A | <0.001*** | 0.003** |
| C) MS 60 mg/NTX 0.01 mg | 20 | 9:37 | (4:02, 21:50) | C-A | <0.001*** | <0.001*** |
| D) MS 60 mg/NTX 0.1 mg | 20 | 10:27 | (5:03, 21:24) | D-A | <0.001*** | <0.001*** |
| E) MS 60 mg/NTX 1 mg | 20 | 2:30 | (1:44, 7:54) | E-A | 0.049* | 0.120 |
| | | | | C-B | 0.465 | 0.382 |
| | | | | D-B | 0.502 | 0.409 |
| | | | | E-B | 0.203 | 0.153 |

*, **, ***P-VALUE <=0.05, <=0.01, or <= <0.001 RESPECTIVELY.

TABLE 20B
Time to Rescue Medication
Intent-To-Treat Population, Male Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| EFFICACY OBSERVATION | | | | | | |
| PERIOD (0-8 HOURS) | | | | | | |
| A) Placebo | 18 | >8:00 | (2:21, >8:00) | Treatment | 0.961 | 0.876 |
| B) MS 60 mg | 18 | >8:00 | (2:01, >8:00) | B-A | N/D | N/D |
| C) MS 60 mg/NTX 0.01 mg | 21 | >8:00 | (2:36, >8:00) | C-A | N/D | N/D |
| D) MS 60 mg/NTX 0.1 mg | 21 | 3:06 | (2:03, >8:00) | D-A | N/D | N/D |
| E) MS 60 mg/NTX 1 mg | 21 | >8:00 | (1:43, >8:00) | E-A | N/D | N/D |
| | | | | C-B | N/D | N/D |
| | | | | D-B | N/D | N/D |
| | | | | E-B | N/D | N/D |
| SAFETY OBSERVATION | | | | | | |
| PERIOD (0-24 HOURS) | | | | | | |
| A) Placebo | 18 | 8:57 | (2:21, 9:51) | Treatment | 0.988 | 0.869 |
| B) MS 60 mg | 18 | 5:41 | (2:01, 17:28) | B-A | N/D | N/D |
| C) MS 60 mg/NTX 0.01 mg | 21 | 9:14 | (2:36, 21:44) | C-A | N/D | N/D |
| D) MS 60 mg/NTX 0.1 mg | 21 | 3:06 | (2:03, 18:17) | D-A | N/D | N/D |
| E) MS 60 mg/NTX 1 mg | 21 | 9:01 | (1:43 17:47) | E-A | N/D | N/D |
| | | | | C-B | N/D | N/D |
| | | | | D-B | N/D | N/D |
| | | | | E-B | N/D | N/D |

*, **, ***P-VALUE <=0.05, <=0.01, or <= <0.001 RESPECTIVELY.

Tables 21A for females and 21B for males present the summary and analysis of percent of subjects who took remedication up to 8 and 24 hours. For females, analysis of the percentage of subjects who remedicated within 8 hours showed the lowest percentage for the low-dose (0.01 mg NTX) and mid-dose (0.1 mg NTX) combination groups. In males, the percentage of subjects remedicating (0-8 hours) was comparable across all treatment groups. Analyses of the percentage of subjects who remedicated within 24 hours indicated that all 5 treatment groups were comparable, however, the data should be interpreted with caution because subjects were not under close supervision after 8 hours.

TABLE 21A
Percent of Subjects Rescued
Intent-To-Treat Population, Female Patients

| | RESCUED | | | |
|---|---|---|---|---|
| TREATMENT | YES | NO | SOURCE | P-VALUE [1] |

EFFICACY OBSERVATION PERIOD (0-8 HOURS)

| | | | | |
|---|---|---|---|---|
| A) Placebo | 19 (86.4%) | 3 (13.6%) | TREATMENT | <0.001** |
| B) MS 60 mg | 11 (47.8%) | 12 (52.2%) | B-A | N/D |
| C) MS 60 mg/NTX 0.01 mg | 9 (45.0%) | 11 (55.0%) | C-A | N/D |
| D) MS 60 mg/NTX 0.1 mg | 7 (35.0%) | 13 (65.0%) | D-A | N/D |
| E) MS 60 mg/NTX 1 mg | 15 (75.0%) | 5 (25.0%) | E-A | N/D |
| | | | C-B | N/D |
| | | | D-B | N/D |
| | | | E-B | N/D |

SAFETY OBSERVATION PERIOD (0-24 HOURS)

| | | | | |
|---|---|---|---|---|
| A) Placebo | 22 (100.0%) | 0 (0.0%) | TREATMENT | 0.182 |
| B) MS 60 mg | 20 (87.0%) | 3 (13.0%) | B-A | N/D |
| C) MS 60 mg/NTX 0.01 mg | 16 (80.0%) | 4 (20.0%) | C-A | N/D |
| D) MS 60 mg/NTX 0.1 mg | 16 (80.0%) | 4 (20.0%) | D-A | N/D |
| E) MS 60 mg/NTX 1 mg | 18 (90.0%) | 2 (10.0%) | E-A | N/D |
| | | | C-B | N/D |
| | | | D-B | N/D |
| | | | E-B | N/D |

N/D: NOT DONE (BECAUSE OVERALL P-VALUE NOT SIGNIFICANT).

TABLE 21B
Percent of Subjects Rescued
Intent-To-Treat Population, Male Patients

| | RESCUED | | | |
|---|---|---|---|---|
| TREATMENT | YES | NO | SOURCE | P-VALUE [1] |

EFFICACY OBSERVATION PERIOD (0-8 HOURS)

| | | | | |
|---|---|---|---|---|
| A) Placebo | 8 (44.4%) | 10 (55.6%) | TREATMENT | 0.962 |
| B) MS 60 mg | 9 (50.0%) | 9 (50.0%) | B-A | N/D |
| C) MS 60 mg/NTX 0.01 mg | 10 (47.6%) | 11 (52.4%) | C-A | N/D |
| D) MS 60 mg/NTX 0.1 mg | 12 (57.1%) | 9 (42.9%) | D-A | N/D |
| E) MS 60 mg/NTX 1 mg | 10 (47.6%) | 11 (52.4%) | E-A | N/D |
| | | | C-B | N/D |
| | | | D-B | N/D |
| | | | E-B | N/D |

SAFETY OBSERVATION PERIOD (0-24 HOURS)

| | | | | |
|---|---|---|---|---|
| A) Placebo | 15 (83.3%) | 3 (16.7%) | TREATMENT | 1.000 |
| B) MS 60 mg | 15 (83.3%) | 3 (16.7%) | B-A | N/D |
| C) MS 60 mg/NTX 0.01 mg | 17 (81.0%) | 4 (19.0%) | C-A | N/D |
| D) MS 60 mg/NTX 0.1 mg | 17 (81.0%) | 4 (19.0%) | D-A | N/D |
| E) MS 60 mg/NTX 1 mg | 17 (81.0%) | 4 (19.0%) | E-A | N/D |
| | | | C-B | N/D |
| | | | D-B | N/D |
| | | | E-B | N/D |

N/D: NOT DONE (BECAUSE OVERALL P-VALUE NOT S1GNIFICANT).

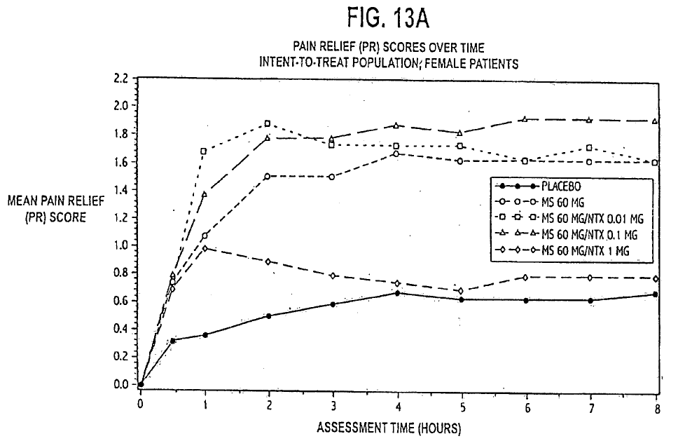

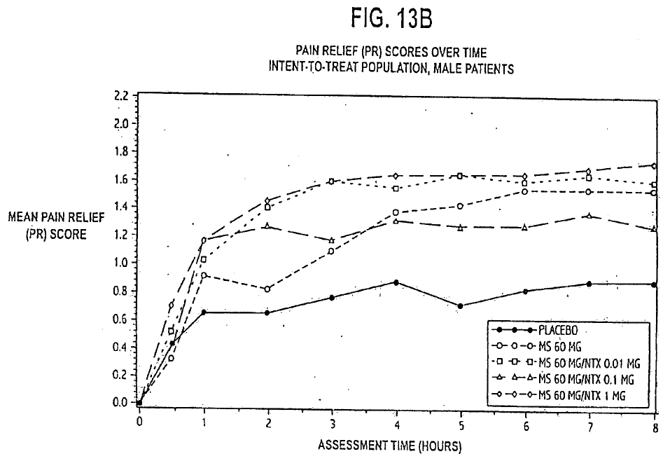

FIGS. 13A for females and 13B for males are visual presentations of the hourly pain relief scores presented in Table 22A for females and 22B for males. The hourly pain relief scores were summarized and analyzed in 2 ways: first as a categorical variable and second as a numerical variable. Because results of these two methods were similar, only the results from the numerical version are presented here. In females, the hourly pain relief scores for the placebo treatment were less than those for the active treatment groups. This was true for males from hour 1 through hour 8. For females and males, there was separation between the placebo and the active treatment groups that continued throughout the 8-hour study period. For females, highest pain relief scores were observed for the low-dose (0.01 mg NTX) and mid-dose (0.1 mg NTX) combination groups (FIG. 13A). For males, highest pain relief scores were observed for the low-dose (0.01 mg NTX) and high-dose (1.0 mg NTX) combination groups.

TABLE 22A
Pain Relief (PR) Scores [1]
Intent-To-Treat Population, Female Patients

| | PAIN RELIEF SCORE (PR) | | | | P-VALUE |
|---|---|---|---|---|---|
| TREATMENT | N | MEAN | SD | SOURCE | [1] |
| 30 MINUTES | | | | | |
| A) Placebo | 22 | 0.32 | 0.646 | TRT | 0.482 |
| B) MS 60 mg | 23 | 0.74 | 1.096 | B-A | N/D |
| C) MS 60 mg/NTX 0.01 | 20 | 0.75 | 0.967 | C-A | N/D |
| mg | | | | | |
| D) MS 60 mg/NTX 0.1 mg | 20 | 0.80 | 1.105 | D-A | N/D |
| E) MS 60 mg/NTX 1 mg | 20 | 0.70 | 0.979 | E-A | N/D |
| | | | | C-B | N/D |
| | | | | D-B | N/D |
| | | | | E-B | N/D |
| 1 HOUR | | | | | |
| A) Placebo | 22 | 0.36 | 0.790 | TRT | 0.002** |
| B) MS 60 mg | 23 | 1.09 | 1.041 | B-A | 0.029* |
| C) MS 60 mg/NTX 0.01 | 20 | 1.70 | 1.380 | C-A | <0.001*** |
| mg | | | | | |
| D) MS 60 mg/NTX 0.1 mg | 20 | 1.40 | 1.188 | D-A | 0.002** |
| E) MS 60 mg/NTX 1 mg | 20 | 1.00 | 1.026 | E-A | 0.062 |
| | | | | C-B | 0.070 |
| | | | | D-B | 0.352 |
| | | | | E-B | 0.795 |
| 2 HOURS | | | | | |
| A) Placebo | 22 | 0.50 | 0.802 | TRT | <0.001*** |
| B) MS 60 mg | 23 | 1.52 | 1.377 | B-A | 0.004** |
| C) MS 60 mg/NTX 0.01 | 20 | 1.90 | 1.252 | C-A | <0.001*** |
| mg | | | | | |
| D) MS 60 mg/NTX 0.1 mg | 20 | 1.80 | 1.281 | D-A | <0.001*** |
| E) MS 60 mg/NTX 1 mg | 20 | 0.90 | 1.165 | E-A | 0.279 |
| | | | | C-B | 0.301 |
| | | | | D-B | 0.446 |
| | | | | E-B | 0.091 |
| 3 HOURS | | | | | |
| A) Placebo | 22 | 0.59 | 0.908 | TRT | 0.004** |
| B) MS 60 mg | 23 | 1.52 | 1.442 | B-A | 0.015* |
| C) MS 60 mg/NTX 0.01 | 20 | 1.75 | 1.333 | C-A | 0.003** |
| mg | | | | | |
| D) MS 60 mg/NTX 0.1 mg | 20 | 1.80 | 1.361 | D-A | 0.002** |
| E) MS 60 mg/NTX 1 mg | 20 | 0.80 | 1.240 | E-A | 0.595 |
| | | | | C-B | 0.557 |
| | | | | D-B | 0.475 |
| | | | | E-B | 0.065 |
| 4 HOURS | | | | | |
| A) Placebo | 22 | 0.68 | 1.086 | TRT | 0.006** |
| B) MS 60 mg | 23 | 1.70 | 1.579 | B-A | 0.016* |
| C) MS 60 mg/NTX 0.01 | 20 | 1.75 | 1.410 | C-A | 0.014* |
| mg | | | | | |
| D) MS 60 mg/NTX 0.1 mg | 20 | 1.90 | 1.553 | D-A | 0.005** |
| E) MS 60 mg/NTX 1 mg | 20 | 0.75 | 1.251 | E-A | 0.874 |
| | | | | C-B | 0.898 |
| | | | | D-B | 0.631* |
| | | | | E-B | 0.028* |
| 5 HOURS | | | | | |
| A) Placebo | 22 | 0.64 | 1.002 | TRT | 0.007** |
| B) MS 60 mg | 23 | 1.65 | 1.613 | B-A | 0.018* |
| C) MS 60 mg/NTX 0.01 | 20 | 1.75 | 1.482 | C-A | 0.012* |
| mg | | | | | |
| D) MS 60 mg/NTX 0.1 mg | 20 | 1.85 | 1.663 | D-A | 0.006** |
| E) MS 60 mg/NTX 1 mg | 20 | 0.70 | 1.218 | E-A | 0.884 |
| | | | | C-B | 0.821 |
| | | | | D-B | 0.648 |
| | | | | E-B | 0.030* |
| 6 HOURS | | | | | |
| A) Placebo | 22 | 0.64 | 1.002 | TRT | 0.015* |
| B) MS 60 mg | 23 | 1.65 | 1.584 | B-A | 0.023* |
| C) MS 60 mg/NTX 0.01 | 20 | 1.65 | 1.531 | C-A | 0.028* |
| mg | | | | | |
| D) MS 60 mg/NTX 0.1 mg | 20 | 1.95 | 1.761 | D-A | 0.004** |
| E) MS 60 mg/NTX 1 mg | 20 | 0.80 | 1.436 | E-A | 0.721 |
| | | | | C-B | 0.996 |
| | | | | D-B | 0.511 |
| | | | | E-B | 0.062 |
| 7 HOURS | | | | | |
| A) Placebo | 22 | 0.64 | 1.002 | TRT | 0.014* |
| B) MS 60 mg | 23 | 1.65 | 1.668 | B-A | 0.026* |
| C) MS 60 mg/NTX 0.01 | 20 | 1.75 | 1.585 | C-A | 0.018* |
| mg | | | | | |
| D) MS 60 mg/NTX 0.1 mg | 20 | 1.95 | 1.761 | D-A | 0.005** |
| E) MS 60 mg/NTX 1 mg | 20 | 0.80 | 1.436 | E-A | 0.726 |
| | | | | C-B | 0.832 |
| | | | | D-B | 0.520 |
| | | | | E-B | 0.067 |
| 8 HOURS | | | | | |
| A) Placebo | 22 | 0.68 | 1.129 | TRT | 0.027* |
| B) MS 60 mg | 23 | 1.65 | 1.668 | B-A | 0.036* |
| C) MS 60 mg/NTX 0.01 | 20 | 1.65 | 1.631 | C-A | 0.044* |
| mg | | | | | |
| D) MS 60 mg/NTX 0.1 mg | 20 | 1.95 | 1.761 | D-A | 0.008** |
| E) MS 60 mg/NTX 1 mg | 20 | 0.80 | 1.436 | E-A | 0.804 |
| | | | | C-B | 0.996 |
| | | | | D-B | 0.528 |
| | | | | E-B | 0.073 |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.
N/D: NOT DONE (BECAUSE OVERALL P-VALUE NOT SIGNIFICANT).

TABLE 22B
Pain Relief (PR) Scores [1]
Intent-To-Treat Population, Male Patients

| | | | | | P- |
|---|---|---|---|---|---|
| PAIN RELIEF SCORE (PR) | | | | | VALUE |
| TREATMENT | N | MEAN | SD | SOURCE | [1] |
| 30 MINUTES | | | | | |
| A) Placebo | 18 | 0.44 | 0.616 | TRT | 0.612 |
| B) MS 60 mg | 18 | 0.33 | 0.594 | B-A | N/D |
| C) MS 60 mg/NTX 0.01 mg | 21 | 0.52 | 0.814 | C-A | N/D |
| D) MS 60 mg/NTX 0.1 mg | 21 | 0.43 | 0.870 | D-A | N/D |
| E) MS 60 mg/NTX 1 mg | 21 | 0.71 | 0.902 | E-A | N/D |
| | | | | C-B | N/D |
| | | | | D-B | N/D |
| | | | | E-B | N/D |
| 1 HOUR | | | | | |
| A) Placebo | 18 | 0.67 | 1.085 | TRT | 0.548 |
| B) MS 60 mg | 18 | 0.94 | 0.726 | B-A | N/D |
| C) MS 60 mg/NTX 0.01 mg | 21 | 1.05 | 1.117 | C-A | N/D |
| D) MS 60 mg/NTX 0.1 mg | 21 | 1.19 | 1.167 | D-A | N/D |
| E) MS 60 mg/NTX 1 mg | 21 | 1.19 | 1.209 | E-A | N/D |
| | | | | C-B | N/D |
| | | | | D-B | N/D |
| | | | | E-B | N/D |
| 2 HOURS | | | | | |
| A) Placebo | 18 | 0.67 | 0.840 | TRT | 0.107 |
| B) MS 60 mg | 18 | 0.83 | 0.924 | B-A | N/D |
| C) MS 60 mg/NTX 0.01 mg | 21 | 1.43 | 1.207 | C-A | N/D |
| D) MS 60 mg/NTX 0.1 mg | 21 | 1.29 | 1.231 | D-A | N/D |
| E) MS 60 mg/NTX 1 mg | 21 | 1.48 | 1.365 | E-A | N/D |
| | | | | C-B | N/D |
| | | | | D-B | N/D |
| | | | | E-B | N/D |
| 3 HOURS | | | | | |
| A) Placebo | 18 | 0.78 | 1.114 | TRT | 0.243 |
| B) MS 60 mg | 18 | 1.11 | 1.183 | B-A | N/D |
| C) MS 60 mg/NTX 0.01 mg | 21 | 1.62 | 1.499 | C-A | N/D |
| D) MS 60 mg/NTX 0.1 mg | 21 | 1.19 | 1.327 | D-A | N/D |
| E) MS 60 mg/NTX 1 mg | 21 | 1.62 | 1.499 | E-A | N/D |
| | | | | C-B | N/D |
| | | | | D-B | N/D |
| | | | | E-B | N/D |
| 4 HOURS | | | | | |
| A) Placebo | 18 | 0.89 | 1.323 | TRT | 0.497 |
| B) MS 60 mg | 18 | 1.39 | 1.420 | B-A | N/D |
| C) MS 60 mg/NTX 0.01 mg | 21 | 1.57 | 1.326 | C-A | N/D |
| D) MS 60 mg/NTX 0.1 mg | 21 | 1.33 | 1.426 | D-A | N/D |
| E) MS 60 mg/NTX 1 mg | 21 | 1.67 | 1.592 | E-A | N/D |
| | | | | C-B | N/D |
| | | | | D-B | N/D |
| | | | | E-B | N/D |
| 5 HOURS | | | | | |
| A) Placebo | 18 | 0.72 | 1.018 | TRT | 0.222 |
| B) MS 60 mg | 18 | 1.44 | 1.464 | B-A | N/D |
| C) MS 60 mg/NTX 0.01 mg | 21 | 1.67 | 1.461 | C-A | N/D |
| D) MS 60 mg/NTX 0.1 mg | 21 | 1.29 | 1.384 | D-A | N/D |
| E) MS 60 mg/NTX 1 mg | 21 | 1.67 | 1.592 | E-A | N/D |
| | | | | C-B | N/D |
| | | | | D-B | N/D |
| | | | | E-B | N/D |
| 6 HOURS | | | | | |
| A) Placebo | 18 | 0.83 | 1.200 | TRT | 0.379 |
| B) MS 60 mg | 18 | 1.56 | 1.542 | B-A | N/D |
| C) MS 60 mg/NTX 0.01 mg | 21 | 1.62 | 1.465 | C-A | N/D |
| D) MS 60 mg/NTX 0.1 mg | 21 | 1.29 | 1.419 | D-A | N/D |
| E) MS 60 mg/NTX 1 mg | 21 | 1.67 | 1.592 | E-A | N/D |
| | | | | C-B | N/D |
| | | | | D-B | N/D |
| | | | | E-B | N/D |
| 7 HOURS | | | | | |
| A) Placebo | 18 | 0.89 | 1.278 | TRT | 0.463 |
| B) MS 60 mg | 18 | 1.56 | 1.542 | B-A | N/D |
| C) MS 60 mg/NTX 0.01 mg | 21 | 1.67 | 1.592 | C-A | N/D |
| D) MS 60 mg/NTX 0.1 mg | 21 | 1.38 | 1.465 | D-A | N/D |
| E) MS 60 mg/NTX 1 mg | 21 | 1.71 | 1.678 | E-A | N/D |
| | | | | C-B | N/D |
| | | | | D-B | N/D |
| | | | | E-B | N/D |
| 8 HOURS | | | | | |
| A) Placebo | 18 | 0.89 | 1.278 | TRT | 0.417 |
| B) MS 60 mg | 18 | 1.56 | 1.542 | B-A | N/D |
| C) MS 60 mg/NTX 0.01 mg | 21 | 1.62 | 1.564 | C-A | N/D |
| D) MS 60 mg/NTX 0.1 mg | 21 | 1.29 | 1.419 | D-A | N/D |
| E) MS 60 mg/NTX 1 mg | 21 | 1.76 | 1.700 | E-A | N/D |
| | | | | C-B | N/D |
| | | | | D-B | N/D |
| | | | | E-B | N/D |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.
N/D: NOT DONE (BECAUSE OVERALL P-VALUE NOT SIGNIFICANT).

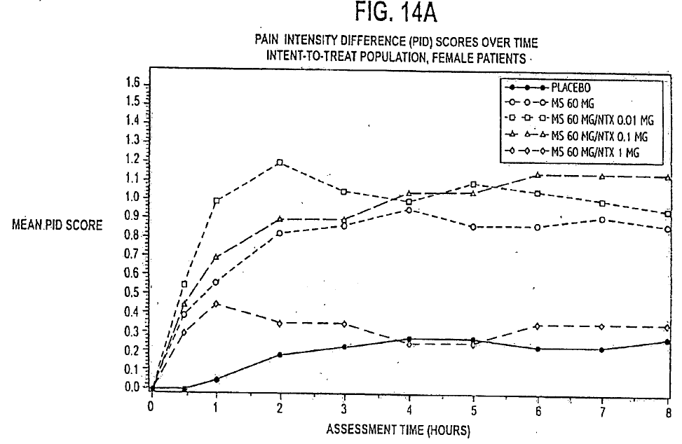

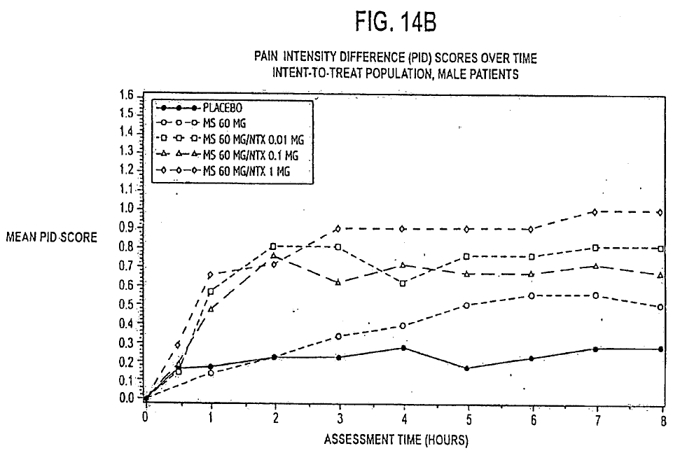

The hourly pain intensity difference (PID) data are presented in Table 23A and FIG. 14A for females and in Table 23B and FIG. 14B for males. For females, the mean scores for the morphine and morphine/naltrexone combination groups were higher than the mean PID scores for the placebo group at each assessment time. The means for the low-dose (0.01 mg NTX) and mid-dose (0.1 mg NTX) combination groups were greater than the means for high-dose (1.0 mg NTX combination) and placebo groups. Highest pain relief as measured by PID scores was observed for the low-dose (0.01 mg NTX) and mid-dose (0.1 mg NTX) combination groups. In males, the highest PID scores were most often observed for the high dose (1.0 mg NTX) combination group.

TABLE 23A
Pain Intensity Difference (PID) Scores
Intent-To-Treat Population, Female Patients

| | PAIN RELIEF SCORE (PR) | | | | P-VALUE |
|---|---|---|---|---|---|
| TREATMENT | N | MEAN | SD | SOURCE | [1] |
| 30 MINUTES | | | | | |
| A) Placebo | 22 | 0.00 | 0.535 | TRT | 0.144 |
| B) MS 60 mg | 23 | 0.39 | 0.722 | B-A | N/D |
| C) MS 60 mg/NTX 0.01 | 20 | 0.55 | 0.759 | C-A | N/D |
| mg | | | | | |
| D) MS 60 mg/NTX 0.1 mg | 20 | 0.45 | 0.759 | D-A | N/D |
| E) MS 60 mg/NTX 1 mg | 20 | 0.30 | 0.865 | E-A | N/D |
| | | | | C-B | N/D |
| | | | | D-B | N/D |
| | | | | E-B | N/D |
| 1 HOUR | | | | | |
| A) Placebo | 22 | 0.05 | 0.722 | TRT | 0.013* |
| B) MS 60 mg | 23 | 0.57 | 0.945 | B-A | 0.050 |
| C) MS 60 mg/NTX 0.01 | 20 | 1.00 | 0.973 | C-A | <0.001*** |
| mg | | | | | |
| D) MS 60 mg/NTX 0.1 mg | 20 | 0.70 | 0.865 | D-A | 0.018* |
| E) MS 60 mg/NTX 1 mg | 20 | 0.45 | 0.887 | E-A | 0.140 |
| | | | | C-B | 0.109 |
| | | | | D-B | 0.618 |
| | | | | E-B | 0.670 |
| 2 HOURS | | | | | |
| A) Placebo | 22 | 0.18 | 0.664 | TRT | <0.001** |
| B) MS 60 mg | 23 | 0.83 | 1.072 | B-A | 0.016* |
| C) MS 60 mg/NTX 0.01 | 20 | 1.20 | 0.834 | C-A | <0.001*** |
| mg | | | | | |
| D) MS 60 mg/NTX 0.1 mg | 20 | 0.90 | 0.788 | D-A | 0.009** |
| E) MS 60 mg/NTX 1 mg | 20 | 0.35 | 0.988 | E-A | 0.539 |
| | | | | C-B | 0.169 |
| | | | | D-B | 0.785 |
| | | | | E-B | 0.081 |
| 3 HOURS | | | | | |
| A) Placebo | 22 | 0.23 | 0.612 | TRT | 0.012* |
| B) MS 60 mg | 23 | 0.87 | 1.100 | B-A | 0.020* |
| C) MS 60 mg/NTX 0.01 | 20 | 1.05 | 0.887 | C-A | 0.004** |
| mg | | | | | |
| D) MS 60 mg/NTX 0.1 mg | 20 | 0.90 | 0.852 | D-A | 0.019* |
| E) MS 60 mg/NTX 1 mg | 20 | 0.35 | 1.040 | E-A | 0.665 |
| | | | | C-B | 0.520 |
| | | | | D-B | 0.913 |
| | | | | E-B | 0.066 |
| 4 HOURS | | | | | |
| A) Placebo | 22 | 0.27 | 0.703 | TRT | 0.007** |
| B) MS 60 mg | 23 | 0.96 | 1.186 | B-A | 0.019* |
| C) MS 60 mg/NTX 0.01 | 20 | 1.00 | 0.918 | C-A | 0.016* |
| mg | | | | | |
| D) MS 60 mg/NTX 0.1 mg | 20 | 1.05 | 0.945 | D-A | 0.010* |
| E) MS 60 mg/NTX 1 mg | 20 | 0.25 | 1.020 | E-A | 0.939 |
| | | | | C-B | 0.883 |
| | | | | D-B | 0.753 |
| | | | | E-B | 0.019* |
| 5 HOURS | | | | | |
| A) Placebo | 22 | 0.27 | 0.703 | TRT | 0.008** |
| B) MS 60 mg | 23 | 0.87 | 1.180 | B-A | 0.047* |
| C) MS 60 mg/NTX 0.01 | 20 | 1.10 | 1.021 | C-A | 0.008** |
| mg | | | | | |
| D) MS 60 mg/NTX 0.1 mg | 20 | 1.05 | 0.999 | D-A | 0.013* |
| E) MS 60 mg/NTX 1 mg | 20 | 0.25 | 1.020 | E-A | 0.941 |
| | | | | C-B | 0.451 |
| | | | | D-B | 0.555 |
| | | | | E-B | 0.044* |
| 6 HOURS | | | | | |
| A) Placebo | 22 | 0.23 | 0.685 | TRT | 0.015* |
| B) MS 60 mg | 23 | 0.87 | 1.140 | B-A | 0.044* |
| C) MS 60 mg/NTX 0.01 | 20 | 1.05 | 1.099 | C-A | 0.013* |
| mg | | | | | |
| D) MS 60 mg/NTX 0.1 mg | 20 | 1.15 | 1.089 | D-A | 0.005** |
| E) MS 60 mg/NTX 1 mg | 20 | 0.35 | 1.226 | E-A | 0.708 |
| | | | | C-B | 0.579 |
| | | | | D-B | 0.389 |
| | | | | E-B | 0.112 |
| 7 HOURS | | | | | |
| A) Placebo | 22 | 0.23 | 0.685 | TRT | 0.019* |
| B) MS 60 mg | 23 | 0.91 | 1.240 | B-A | 0.034* |
| C) MS 60 mg/NTX 0.01 | 20 | 1.00 | 1.026 | C-A | 0.021 |
| mg | | | | | |
| D) MS 60 mg/NTX 0.1 mg | 20 | 1.15 | 1.089 | D-A | 0.006** |
| E) MS 60 mg/NTX 1 mg | 20 | 0.35 | 1.226 | E-A | 0.711 |
| | | | | C-B | 0.791 |
| | | | | D-B | 0.471 |
| | | | | E-B | 0.088 |
| 8 HOURS | | | | | |
| A) Placebo | 22 | 0.27 | 0.827 | TRT | 0.042* |
| B) MS 60 mg | 23 | 0.87 | 1.254 | B-A | 0.071 |
| C) MS 60 mg/NTX 0.01 | 20 | 0.95 | 1.050 | C-A | 0.049* |
| mg | | | | | |
| D) MS 60 mg/NTX 0.1 mg | 20 | 1.15 | 1.089 | D-A | 0.011* |
| E) MS 60 mg/NTX 1 mg | 20 | 0.35 | 1.226 | E-A | 0.820 |
| | | | | C-B | 0.811 |
| | | | | D-B | 0.406 |
| | | | | E-B | 0.125 |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.
N/D: NOT DONE (BECAUSE OVERALL P-VALUE NOT SIGNIFICANT).

TABLE 23B
Pain Intensity Difference (PID) Scores
Intent-To-Treat Population, Male Patients

| | | | | | P- |
|---|---|---|---|---|---|
| | PAIN RELIEF SCORE (PR) | | | | VALUE |
| TREATMENT | N | MEAN | SD | SOURCE | [1] |
| 30 MINUTES | | | | | |
| A) Placebo | 18 | 0.17 | 0.618 | TRT | 0.378 |
| B) MS 60 mg | 18 | −0.11 | 0.471 | B-A | N/D |
| C) MS 60 mg/NTX 0.01 mg | 21 | 0.14 | 0.727 | C-A | N/D |
| D) MS 60 mg/NTX 0.1 mg | 21 | 0.19 | 0.512 | D-A | N/D |
| E) MS 60 mg/NTX 1 mg | 21 | 0.29 | 0.717 | E-A | N/D |
| | | | | C-B | N/D |
| | | | | D-B | N/D |
| | | | | E-B | N/D |
| 1 HOUR | | | | | |
| A) Placebo | 18 | 0.17 | 0.786 | TRT | 0.244 |
| B) MS 60 mg | 18 | 0.13 | 0.761 | B-A | N/D |
| C) MS 60 mg/NTX 0.01 mg | 21 | 0.57 | 1.028 | C-A | N/D |
| D) MS 60 mg/NTX 0.1 mg | 21 | 0.48 | 0.814 | D-A | N/D |
| E) MS 60 mg/NTX 1 mg | 21 | 0.67 | 1.107 | E-A | N/D |
| | | | | C-B | N/D |
| | | | | D-B | N/D |
| | | | | E-B | N/D |
| 2 HOURS | | | | | |
| A) Placebo | 18 | 0.22 | 0.647 | TRT | 0.124 |
| B) MS 60 mg | 18 | 0.22 | 0.808 | B-A | N/D |
| C) MS 60 mg/NTX 0.01 mg | 21 | 0.81 | 1.123 | C-A | N/D |
| D) MS 60 mg/NTX 0.1 mg | 21 | 0.76 | 0.889 | D-A | N/D |
| E) MS 60 mg/NTX 1 mg | 21 | 0.71 | 1.146 | E-A | N/D |
| | | | | C-B | N/D |
| | | | | D-B | N/D |
| | | | | E-B | N/D |
| 3 HOURS | | | | | |
| A) Placebo | 18 | 0.22 | 0.732 | TRT | 0.215 |
| B) MS 60 mg | 18 | 0.33 | 0.970 | B-A | N/D |
| C) MS 60 mg/NTX 0.01 mg | 21 | 0.81 | 1.250 | C-A | N/D |
| D) MS 60 mg/NTX 0.1 mg | 21 | 0.62 | 0.921 | D-A | N/D |
| E) MS 60 mg/NTX 1 mg | 21 | 0.90 | 1.300 | E-A | N/D |
| | | | | C-B | N/D |
| | | | | D-B | N/D |
| | | | | E-B | N/D |
| 4 HOURS | | | | | |
| A) Placebo | 18 | 0.28 | 0.826 | TRT | 0.372 |
| B) MS 60 mg | 18 | 0.39 | 1.037 | B-A | N/D |
| C) MS 60 mg/NTX 0.01 mg | 21 | 0.62 | 0.973 | C-A | N/D |
| D) MS 60 mg/NTX 0.1 mg | 21 | 0.71 | 1.007 | D-A | N/D |
| E) MS 60 mg/NTX 1 mg | 21 | 0.90 | 1.375 | E-A | N/D |
| | | | | C-B | N/D |
| | | | | D-B | N/D |
| | | | | E-B | N/D |
| 5 HOURS | | | | | |
| A) Placebo | 18 | 0.17 | 0.618 | TRT | 0.260 |
| B) MS 60 mg | 18 | 0.50 | 1.150 | B-A | N/D |
| C) MS 60 mg/NTX 0.01 mg | 21 | 0.76 | 1.091 | C-A | N/D |
| D) MS 60 mg/NTX 0.1 mg | 21 | 0.67 | 0.966 | D-A | N/D |
| E) MS 60 mg/NTX 1 mg | 21 | 0.90 | 1.338 | E-A | N/D |
| | | | | C-B | N/D |
| | | | | D-B | N/D |
| | | | | E-B | N/D |
| 6 HOURS | | | | | |
| A) Placebo | 18 | 0.22 | 0.647 | TRT | 0.378 |
| B) MS 60 mg | 18 | 0.56 | 1.199 | B-A | N/D |
| C) MS 60 mg/NTX 0.01 mg | 21 | 0.76 | 1.136 | C-A | N/D |
| D) MS 60 mg/NTX 0.1 mg | 21 | 0.67 | 0.966 | D-A | N/D |
| E) MS 60 mg/NTX 1 mg | 21 | 0.90 | 1.338 | E-A | N/D |
| | | | | C-B | N/D |
| | | | | D-B | N/D |
| | | | | E-B | N/D |
| 7 HOURS | | | | | |
| A) Placebo | 18 | 0.28 | 0.752 | TRT | 0.384 |
| B) MS 60 mg | 18 | 0.56 | 1.199 | B-A | N/D |
| C) MS 60 mg/NTX 0.01 mg | 21 | 0.81 | 1.250 | C-A | N/D |
| D) MS 60 mg/NTX 0.1 mg | 21 | 0.71 | 1.007 | D-A | N/D |
| E) MS 60 mg/NTX 1 mg | 21 | 1.00 | 1.449 | E-A | N/D |
| | | | | C-B | N/D |
| | | | | D-B | N/D |
| | | | | E-B | N/D |
| 8 HOURS | | | | | |
| A) Placebo | 18 | 0.28 | 0.752 | TRT | 0.345 |
| B) MS 60 mg | 18 | 0.50 | 1.200 | B-A | N/D |
| C) MS 60 mg/NTX 0.01 mg | 21 | 0.81 | 1.250 | C-A | N/D |
| D) MS 60 mg/NTX 0.1 mg | 21 | 0.67 | 0.966 | D-A | N/D |
| E) MS 60 mg/NTX 1 mg | 21 | 1.00 | 1.414 | E-A | N/D |
| | | | | C-B | N/D |
| | | | | D-B | N/D |
| | | | | E-B | N/D |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.
N/D: NOT DONE (BECAUSE OVERALL P-VALUE NOT SIGNIFICANT).

The mean MAXPAR scores are presented in Table 24A for females and 24C for males. In females, the mean MAXPAR scores were highest for the low-dose (0.01 mg NTX) and mid-dose (0.1 mg NTX) combination groups compared to all other groups. The mean scores for the low-dose and mid-dose groups were greater than the mean score for the morphine group, which in turn, was greater than the mean score for the placebo group. In males, the mean MAXPAR scores were highest for the high-dose (1.0 mg NTX) and low-dose (0.01 mg NTX) combination groups.

The mean PEAKPID scores presented in Table 24B for females and 24D for males were different among treatment groups, and were greater for the morphine/naltrexone groups compared to the placebo group. In females, the mean PEAKPID scores for the low-dose (0.01 mg NTX) and mid-dose (0.1 mg NTX) combination groups were highest. In males, the high-dose (1.0 mg NTX) and low-dose (0.01 mg NTX) combination groups had the highest mean PEAKPID scores.

TABLE 24A
Maximum Pain Relief Scores (MAXPAR)
Intent-To-Treat Population, Female Patients

| MAXIMUM PAIN RELIEF SCORE | | | | | | | | |
|---|---|---|---|---|---|---|---|---|
| TREATMENT | N | MEAN | SD | MIN | MEDIAN | MAX | SOURCE | P-VALUE [1] |
| A) Placebo | 22 | 0.91 | 1.342 | 0.0 | 0.0 | 4.0 | TRT | <0.001*** |
| B) MS 60 mg | 23 | 2.04 | 1.637 | 0.0 | 3.0 | 4.0 | B-A | 0.009** |
| C) MS 60 mg/NTX 0.01 mg | 20 | 2.80 | 1.281 | 0.0 | 3.0 | 4.0 | C-A | <0.001*** |
| D) MS 60 mg/NTX 0.1 mg | 20 | 2.40 | 1.501 | 0.0 | 3.0 | 4.0 | D-A | <0.001** |
| E) MS 60 mg/NTX 1 mg | 20 | 1.40 | 1.429 | 0.0 | 1.0 | 4.0 | E-A | 0.275 |
| | | | | | | | C-B | 0.090 |
| | | | | | | | D-B | 0.422 |
| | | | | | | | E-B | 0.149 |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.

TABLE 24B
Peak Pain Intensity Differences (PEAKPID)
Intent-To-Treat Population, Female Patients

| PEAK PAIN INTENSITY DIFFERENCES | | | | | | | | |
|---|---|---|---|---|---|---|---|---|
| TREATMENT | N | MEAN | SD | MIN | MEDIAN | MAX | SOURCE | P-VALUE [1] |
| A) Placebo | 22 | 0.50 | 0.913 | −1 | 0.0 | 3 | TRT | <0.001*** |
| B) MS 60 mg | 23 | 1.35 | 1.071 | 0 | 1.0 | 3 | B-A | 0.005** |
| C) MS 60 mg/NTX 0.01 mg | 20 | 1.70 | 0.923 | 0 | 2.0 | 3 | C-A | <0.001*** |
| D) MS 60 mg/NTX 0.1 mg | 20 | 1.40 | 0.940 | 0 | 1.5 | 3 | D-A | 0.004** |
| E) MS 60 mg/NTX 1 mg | 20 | 0.70 | 1.174 | −1 | 0.0 | 3 | E-A | 0.522 |
| | | | | | | | C-B | 0.256 |
| | | | | | | | D-B | 0.866 |
| | | | | | | | E-B | 0.038* |

[1] FROM ONE-WAY ANALYSIS OF VARIANCE AND FISHER'S PROTECTED LEAST SIGNIFICANT DIFFERENCE TEST.
*, **, ***P-VALUE <=0.05, <=0.01, or <= <0.001 RESPECTIVELY.

TABLE 24C
Maximum Pain Relief Scores (MAXPAR)
Intent-To-Treat Population, Male Patients

| MAXIMUM PAIN RELIEF SCORE | | | | | | | | |
|---|---|---|---|---|---|---|---|---|
| TREATMENT | N | MEAN | SD | MIN | MEDIAN | MAX | SOURCE | P-VALUE [1] |
| A) Placebo | 18 | 1.33 | 1.372 | 0.0 | 1.0 | 4.0 | TRT | 0.674 |
| B) MS 60 mg | 18 | 1.83 | 1.425 | 0.0 | 2.5 | 4.0 | B-A | N/D |
| C) MS 60 mg/NTX 0.01 mg | 21 | 2.00 | 1.673 | 0.0 | 3.0 | 4.0 | C-A | N/D |
| D) MS 60 mg/NTX 0.1 mg | 21 | 1.81 | 1.401 | 0.0 | 2.0 | 4.0 | D-A | N/D |
| E) MS 60 mg/NTX 1 mg | 21 | 2.00 | 1.789 | 0.0 | 2.0 | 4.0 | E-A | N/D |
| | | | | | | | C-B | N/D |
| | | | | | | | D-B | N/D |
| | | | | | | | E-B | N/D |

[1] FROM ONE-WAY ANALYSIS OF VARIANCE AND FISHER'S PROTECTED LEAST SIGNIFICANT DIFFERENCE TEST.
*, **, ***: P-VALUE <=0.05, <=0.01, or <=<0.001 RESPECTIVELY.

TABLE 24D
Peak Pain Intensity Differences (PEAKPID)
Intent-To-Treat Population, Male Patients

| PEAK PAIN INTENSITY DIFFERENCES | | | | | | | | |
|---|---|---|---|---|---|---|---|---|
| TREATMENT | N | MEAN | SD | MIN | MEDIAN | MAX | SOURCE | P-VALUE [1] |
| A) Placebo | 18 | 0.56 | 0.856 | −1 | 1.0 | 2 | TRT | 0.302 |
| B) MS 60 mg | 18 | 0.78 | 1.003 | −1 | 1.0 | 2 | B-A | N/D |
| C) MS 60 mg/NTX 0.01 mg | 21 | 1.14 | 1.276 | −1 | 1.0 | 3 | C-A | N/D |
| D) MS 60 mg/NTX 0.1 mg | 21 | 0.95 | 1.071 | −1 | 1.0 | 3 | D-A | N/D |
| E) MS 60 mg/NTX 1 mg | 21 | 1.29 | 1.384 | −1 | 2.0 | 3 | E-A | N/D |
| | | | | | | | C-B | N/D |
| | | | | | | | D-B | N/D |
| | | | | | | | D-B | N/D |

[1] FROM ONE-WAY ANALYSIS OF VARIANCE AND FISHER'S PROTECTED LEAST SIGNIFICANT DIFFERENCE TEST.
*, **, ***: P-VALUE <=0.05, <=0.01, or <=<0.001 RESPECTIVELY.

Tables 25A for females and 25B for males present the summary and analysis of global evaluations. For both females and males, the placebo treatment had the highest number of subjects who had poor global evaluation scores based on subject evaluation. For females, the low-dose (0.01 mg NTX) and mid-dose (0.1 mg NTX) combination groups were most often rated as “excellent.” For males, the high-dose (1.0 mg NTX) combination group was most often rated as “excellent.” The profiles of the global evaluations scores are based on subjects' evaluations.

TABLE 25A
Global Evaluation of Study Medication
Intent-To-Treat Population, Female Patients

| | | | | | VERY | | | | | |
|---|---|---|---|---|---|---|---|---|---|---|
| | | POOR | FAIR | GOOD | GOOD | EXCELLENT | | | | |
| TREATMENT | N | (0) | (1) | (2) | (3) | (4) | MEAN | (SD) | SOURCE | P-VALUE [1] |
| A) Placebo | 22 | 17 (77.3%) | 1 (4.5%) | 2 (9.1%) | 2 (9.1%) | 0 (0.0%) | 0.5 | (1.01) | TRT | <0.001** |
| B) MS 60 mg | 23 | 9 (39.1%) | 1 (4.3%) | 4 (17.4%) | 7 (30.4%) | 2 (8.7%) | 1.7 | (1.50) | B-A | 0.005** |
| C) MS 60 mg/NTX | 20 | 4 (20.0%) | 1 (5.0%) | 6 (30.0%) | 6 (30.0%) | 3 (15.0%) | 2.2 | (1.35) | C-A | <0.001*** |
| 0.01 mg | | | | | | | | | | |
| D) MS 60 mg/NTX 0.1 mg | 20 | 6 (30.0%) | 3 (15.0%) | 2 (10.0%) | 6 (30.0%) | 3 (15.0%) | 1.9 | (1.53) | D-A | 0.002** |
| E) MS 60 mg/NTX 1 mg | 20 | 12 (60.0%) | 0 (0.0%) | 4 (20.0%) | 4 (20.0%) | 0 (0.0%) | 1.0 | (1.30) | E-A | 0.166 |
| | | | | | | | | | C-B | 0.256 |
| | | | | | | | | | D-B | 0.665 |
| | | | | | | | | | E-B | 0.135 |

[1] FROM COCHRAN-MANTEL-HAENZEL TEST FOR RAW MEAN SCORES DIFFERENCE.
*, **, ***P-VALUE <=0.05, <=0.01, OR <=<0.001 RESPECTIVELY.

TABLE 25B
Global Evaluation of Study Medication
Intent-To-Treat Population, Male Patients

| | | | | | VERY | | | | | |
|---|---|---|---|---|---|---|---|---|---|---|
| | | POOR | FAIR | GOOD | GOOD | EXCELLENT | | | | |
| TREATMENT | N | (0) | (1) | (2) | (3) | (4) | MEAN | (SD) | SOURCE | P-VALUE [1] |
| A) Placebo | 18 | 11 (61.1%) | 1 (5.6%) | 2 (11.1%) | 4 (22.2%) | 0 (0.0%) | 0.9 | (1.30) | TRT | 0.488 |
| B) MS 60 mg | 18 | 8 (41.4%) | 2 (11.1%) | 4 (22.2%) | 3 (16.7%) | 1 (5.6%) | 1.3 | (1.36) | B-A | N/D |
| C) MS 60 mg/NTX | 21 | 7 (33.3%) | 2 (9.5%) | 3 (14.3%) | 8 (38.1%) | 0 (0.0%) | 1.6 | (1.35) | C-A | N/D |
| 0.01 mg | | | | | | | | | | |
| D) MS 60 mg/NTX 0.1 mg | 21 | 8 (38.1%) | 5 (23.8%) | 5 (23.8%) | 3 (14.3%) | 0 (0.0%) | 1.1 | (1.11) | D-A | N/D |
| E) MS 60 mg/NTX 1 mg | 21 | 8 (38.1%) | 2 (9.5%) | 6 (28.6%) | 1 (4.8%) | 4 (19.0%) | 1.6 | (1.54) | E-A | N/D |
| | | | | | | | | | C-B | N/D |
| | | | | | | | | | D-B | N/D |
| | | | | | | | | | E-B | N/D |

[1] FROM COCHRAN-MANTEL-HAENZEL TEST FOR RAW MEAN SCORES DIFFERENCE.
*, **, ***: P-VALUE <=0.05, <=0.01, OR <=<0.001 RESPECTIVELY.

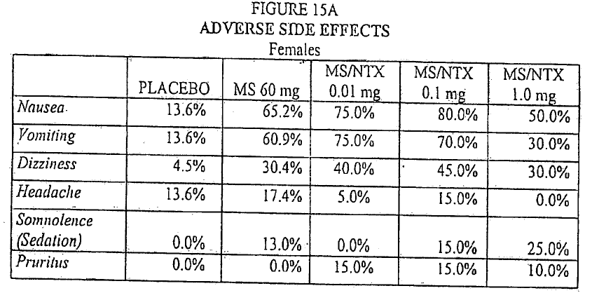

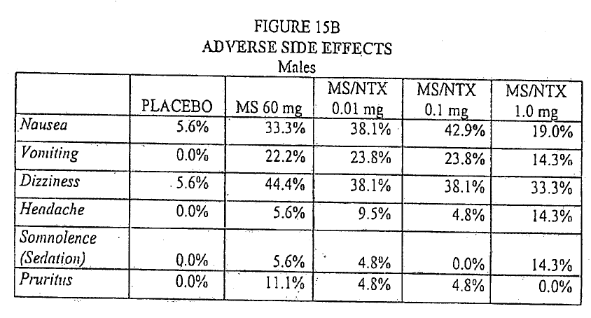

The majority of adverse events reported were categorized as digestive (nausea or vomiting) or nervous system (dizziness or somnolence) as further shown in Tables 26A or 26B for females and 26C or 26D for males. FIGS. 15A for females and 15B for males represent a summary of exemplary adverse side effects according to methods and compositions of the invention.

In females, the placebo group had the lowest incidence of nausea, vomiting, dizziness and somnolence (sedation). For nausea, vomiting and dizziness, the 1.0 mg NTX combination group had the lowest incidence of adverse events compared to the other active treatment groups. For somnolence, the 0.01 mg NTX combination group had the lowest incidence among the active treatment groups.

In males, the placebo group showed the lowest incidence of adverse events. Among the active treatment groups, the 1.0 mg NTX combination group had the lowest incidence of adverse events. Except for somnolence which was lowest in the 0.1 mg NTX combination group.

TABLE 26A
ADVERSE EVENTS BY BODY SYSTEM AND SEVERITY
SAFETY POPULATION, FEMALE PATIENTS

| BODY | | | | | | | | | |
|---|---|---|---|---|---|---|---|---|---|
| SYSTEM | | | | | | | | | |
| ADVERSE | | TOTAL | | | | | | | |
| EVENTS | | NO. OF | NO. OF | | | Number | | | |
| (COSTART | | SUB- | SUBJECTS | | P-Value | Of | Severity [2] | | |
| ENGLISH) | TREATMENT | JECTS | W/EVENT | SOURCE | [1] | Events | MILD | Moderate | SEVERE |

TOTAL NUMBER OF EVENTS
ADVERSE EVENTS
(ALL BODY SYSTEMS)

| | | | | | | | | |
|---|---|---|---|---|---|---|---|---|
| A) PLACEBO | 22 | 7 (31.8%) | Treatment | <0.001*** | 12 | 4 (33.3%) | 3 (25.0%) | 5 (41.7%) |
| B) MS 60 mg | 23 | 22 (95.7%) | A-B | <0.001*** | 55 | 18 (32.7%) | 20 (36.4%) | 17 (30.9%) |
| C) MS 60 mg/NTX 0.01 mg | 20 | 19 (95.0%) | A-C | <0.001*** | 58 | 13 (22.4%) | 24 (41.4%) | 21 (36.2%) |
| D) MS 60 mg/NTX 0.1 mg | 20 | 20 (100.0%) | A-D | <0.001*** | 68 | 17 (25.0%) | 25 (36.8%) | 26 (38.2%) |
| E) MS 60 mg/NTX 1 mg | 20 | 17 (85.0%) | A-E | <0.001*** | 34 | 16 (47.1%) | 10 (29.4%) | 8 (23.5%) |

BODY AS A WHOLE

| | | | | | | | | | |
|---|---|---|---|---|---|---|---|---|---|
| ALL EVENTS | A) PLACEBO | 22 | 3 (13.6%) | Treatment | 0.284 | 3 | 1 (33.3%) | 2 (66.7%) | 0 |
| | B) MS 60 mg | 23 | 4 (17.4%) | | | 5 | 2 (40.05) | 3 (60.05) | 0 |
| | C) MS 60 mg/NTX 0.01 mg | 20 | 3 (15.0%) | | | 3 | 1 (33.3%) | 1 (33.3%) | 1 (33.3%) |
| | D) MS 60 mg/NTX 0.1 mg | 20 | 4 (20.0%) | | | 7 | 1 (14.3%) | 4 (57.1%) | 2 (28.6%) |
| | E) MS 60 mg/NTX 1 mg | 20 | 0 | | | 0 | 0 | 0 | 0 |
| ABDOMINAL | A) PLACEBO | 22 | 0 | Treatment | 0.412 | 0 | 0 | 0 | 0 |
| PAIN | B) MS 60 mg | 23 | 0 | | | 0 | 0 | 0 | 0 |
| | C) MS 60 mg/NTX 0.01 mg | 20 | 1 (5.0%) | | | 1 | 0 | 0 | 1 (100.0%) |
| | D) MS 60 mg/NTX 0.1 mg | 20 | 1 (5.0%) | | | 1 | 0 | 0 | 1 (100.0%) |
| | E) MS 60 mg/NTX 1 mg | 20 | 0 | | | 0 | 0 | 0 | 0 |
| ASTHENIA | A) PLACEBO | 22 | 0 | Treatment | 0.571 | 0 | 0 | 0 | 0 |
| | B) MS 60 mg | 23 | 0 | | | 0 | 0 | 0 | 0 |
| | C) MS 60 mg/NTX 0.01 mg | 20 | 0 | | | 0 | 0 | 0 | 0 |
| | D) MS 60 mg/NTX 0.1 mg | 20 | 1 (5.0%) | | | 1 | 0 | 1 (100.0%) | 0 |
| | E) MS 60 mg/NTX 1 mg | 20 | 0 | | | 0 | 0 | 0 | 0 |
| HEADACHE | A) PLACEBO | 22 | 3 (13.6%) | Treatment | 0.279 | 3 | 1 (33.3%) | 2 (66.7%) | 0 |
| | B) MS 60 mg | 23 | 4 (17.4%) | | | 4 | 1 (25.0%) | 3 (75.0% a0 | 0 |
| | C) MS 60 mg/NTX 0.01 mg | 20 | 1 (5.0%) | | | 1 | 1 (100.0%) | 0 | 0 |
| | D) MS 60 mg/NTX 0.1 mg | 20 | 3 (15.0%) | | | 5 | 1 (20.0%) | 3 (60.0%) | 1 (20.0%) |
| | E) MS 60 mg/NTX 1 mg | 20 | 0 | | | 0 | 0 | 0 | 0 |
| INJECTION | A) PLACEBO | 22 | 0 | Treatment | 1.000 | 0 | 0 | 0 | 0 |
| SITE | B) MS 60 mg | 23 | 1 (4.3%) | | | 1 | 1 (100.0%) | 0 | 0 |
| HEMOR- | C) MS 60 mg/NTX 0.01 mg | 20 | 0 | | | 0 | 0 | 0 | 0 |
| RHAGE | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |
| PAIN | A) PLACEBO | 22 | 0 | Treatment | 0.571 | 0 | 0 | 0 | 0 |
| | B) MS 60 mg | 23 | 0 | | | 0 | 0 | 0 | 0 |
| | C) MS 60 mg/NTX 0.01 mg | 20 | 1 (5.0%) | | | 1 | 0 | 1 (100.0%) | 0 |
| | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |

CARDIOVASCULAR

| | | | | | | | | | |
|---|---|---|---|---|---|---|---|---|---|
| ALL EVENTS | A) PLACEBO | 22 | 0 | Treatment | 0.201 | 0 | 0 | 0 | 0 |
| | B) MS 60 mg | 23 | 2 (8.7%) | | | 2 | 1 (50.0%) | 1 (50.0%) | 0 |
| | C) MS 60 mg/NTX 0.01 mg | 20 | 3 (15.0%) | | | 3 | 1 (33.3%) | 1 (33.3%) | 1 (33.3%) |
| | D) MS 60 mg/NTX 0.1 mg | 20 | 2 (10.0%) | | | 2 | 1 (50.0%) | 1 (50.0%)a | 0 |
| | E) mg 60 mg/NTX 1 mg | 20 | 0 | | | 0 | 0 | 0 | 0 |
| VASODILA- | A) PLACEBO | 22 | 0 | Treatment | 0.201 | 0 | 0 | 0 | 0 |
| TATION | B) MS 60 mg | 23 | 2 (8.7%) | | | 2 | 1 (50.0%) | 1 (50.0%) | 0 |
| | C) MS 60 mg/NTX 0.01 mg | 20 | 3 (15.0%) | | | 3 | 1 (33.3%) | 1 (33.3%) | 1 (33.3%) |
| | D) MS 60 mg/NTX 0.1 mg | 20 | 2 (10.0%) | | | 2 | 1 (50.0%) | 1 (50.0%)a | 0 |
| | E) MS 60 mg/NTX 1 mg | 20 | 0 | | | 0 | 0 | 0 | 0 |

DIGESTIVE

| | | | | | | | | | |
|---|---|---|---|---|---|---|---|---|---|
| ALL EVENTS | A) PLACEBO | 22 | 4 (18.2%) | Treatment | <0.001*** | 7 | 1 (14.3%) | 1 (14.3%) | 5 (71.4%) |
| | B) MS 60 mg | 23 | 16 (69.6%) | A-B | <0.001*** | 30 | 4 (13.3%) | 10 (33.3%) | 16 (53.3%) |
| | C) MS 60 mg/NTX 0.01 mg | 20 | 17 (85.0%) | A-C | <0.001*** | 31 | 4 (12.9%) | 11 (35.5%) | 16 (51.6%) |
| | D) MS 60 mg/NTX 0.1 mg | 20 | 18 (90.0%) | A-D | <0.001*** | 33 | 6 (18.2%) | 7 (21.2%) | 20 (60.6%) |
| | E) MS 60 mg/NTX 1 mg | 20 | 11 (55.0%) | A-E | 0.023* | 18 | 5 (27.8%) | 5 (27.8%) | 8 (44.4%) |
| | | | | D-E | 0.030* | | | | |
| DIARRHEA | A) PLACEBO | 22 | 0 | Treatment | 0.104 | 0 | 0 | 0 | 0 |
| | B) MS 60 mg | 23 | 0 | | | 0 | 0 | 0 | 0 |
| | C) MS 60 mg/NTX 0.01 mg | 20 | 0 | | | 0 | 0 | 0 | 0 |
| | D) MS 60 mg/NTX 0.1 mg | 20 | 2 (10.0%) | | | 2 | 1 (50.0%) | 1 (50.0%) | 0 |
| | E) MS 60 mg/NTX 1 mg | 20 | 0 | | | 0 | 0 | 0 | 0 |
| DYSPEPSIA | A) PLACEBO | 22 | 1 (4.5%) | Treatment | 0.654 | 1 | 1 (100.0%) | 0 | 0 |
| | B) MS 60 mg | 23 | 0 | | | 0 | 0 | 0 | 0 |
| | C) MS 60 mg/NTX 0.01 mg | 20 | 0 | | | 0 | 0 | 0 | 0 |
| | D) MS 60 mg/NTX 0.1 mg | 20 | 0 | | | 0 | 0 | 0 | 0 |
| | E) MS 60 mg/NTX 1 mg | 20 | 1 (5.0%) | | | 1 | 1 (100.0%) | 0 | 0 |
| NAUSEA | A) PLACEBO | 22 | 3 (13.6%) | Treatment | <0.001*** | 3 | 0 | 1 (33.3%) | 2 (66.7%) |
| | B) MS 60 mg | 23 | 15 (65.2%) | A-B | <0.001*** | 16 | 4 (25.0%) | 10 (62.5%) | 2 (12.5%) |
| | C) MS 60 mg/NTX 0.01 mg | 20 | 15 (75.0%) | A-C | <0.001*** | 16 | 4 (25.0%) | 11 (68.8%) | 1 (6.3%) |
| | D) MS 60 mg/NTX 0.1 mg | 20 | 16 (80.0%) | A-D | <0.001*** | 17 | 5 (29.4%) | 6 (35.3%) | 6 (35.3%) |
| | E) MS 60 mg/NTX 1 mg | 20 | 10 (50.0%) | A-E | 0.018* | 11 | 4 (36.4%) | 5 (45.5%) | 2 (18.2%) |
| VOMITING | A) PLACEBO | 22 | 3 (13.6%) | Treatment | <0.001*** | 3 | 0 | 0 | 3 (100.0%) |
| | B) MS 60 mg | 23 | 14 (60.9%) | A-B | <0.001** | 14 | 0 | 0 | 14 (100.0%) |
| | C) MS 60 mg/NTX 0.01 mg | 20 | 15 (75.0%) | A-C | <0.001*** | 15 | 0 | 0 | 15 (100.0%) |
| | D) MS 60 mg/NTX 0.1 mg | 20 | 14 (70.0%) | A-D | <0.001*** | 14 | 0 | 0 | 14 (100.0%) |
| | E) MS 60 mg/NTX 1 mg | 20 | 6 (30.0%) | C-E | 0.010* | 6 | 0 | 0 | 6 (100.0%) |
| | | | | D-E | 0.025* | | | | |

NERVOUS SYSTEM

| | | | | | | | | | |
|---|---|---|---|---|---|---|---|---|---|
| ALL EVENTS | A) PLACEBO | 22 | 1 (4.5%) | Treatment | <0.001*** | 1 | 1 (100.0%) | 0 | 0 |
| | B) MS 60 mg | 23 | 10 (43.5%) | A-B | 0.004** | 14 | 7 (50.0%) | 6 (42.9%) | 1 (7.1%) |
| | C) MS 60 mg/NTX 0.01 mg | 20 | 12 (60.0%) | A-C | <0.001*** | 14 | 4 (28.6%) | 7 (50.0%) | 3 (21.4%) |
| | D) MS 60 mg/NTX 0.1 mg | 20 | 12 (60.0%) | A-D | <0.001*** | 19 | 6 (31.6%) | 9 (47.4%) | 4 (21.1%) |
| | E) MS 60 mg/NTX 1 mg | 20 | 10 (50.0%) | A-E | <0.001** | 12 | 8 (66.7%) | 4 (33.3%) | 0 |
| DIZZINESS | A) PLACEBO | 22 | 1 (4.5%) | Treatment | 0.022* | 1 | 1 (100.0%) | 0 | 0 |
| | B) MS 60 mg | 23 | 7 (30.4%) | A-B | 0.046* | 9 | 5 (55.6%) | 3 (33.3%) | 1 (11.1%) |
| | C) MS 60 mg/NTX 0.01 mg | 20 | 8 (40.0%) | A-C | 0.007** | 8 | 3 (37.5%) | 4 (50.0%) | 1 (12.5%) |
| | D) MS 60 mg/NTX 0.1 mg | 20 | 9 (45.0%) | A-D | 0.003** | 12 | 5 (41.7%) | 4 (33.3%) | 3 (25.0%) |
| | E) MS 60 mg/NTX 1 mg | 20 | 6 (30.0%) | A-E | 0.040* | 6 | 4 (66.7%) | 2 (33.3%) | 0 |
| EUPHORIA | A) PLACEBO | 22 | 0 | Treatment | 0.007** | 0 | 0 | 0 | 0 |
| | B) MS 60 mg | 23 | 0 | A-C | 0.043* | 0 | 0 | 0 | 0 |
| | C) MS 60 mg/NTX 0.01 mg | 20 | 4 (20.0%) | B-C | 0.039* | 4 | 0 | 3 (75.0%) | 1 (25.0%) |
| | D) MS 60 mg/NTX 0.1 mg | 20 | 1 (5.0%) | | | 1 | 0 | 1 (100.0%) | 0 |
| | E) MS 60 mg/NTX 1 mg | 20 | 0 | | | 0 | 0 | 0 | 0 |
| HALLUCI- | A) PLACEBO | 22 | 0 | Treatment | 1.000 | 0 | 0 | 0 | 0 |
| NATIONS | B) MS 60 mg | 23 | 1 (4.3%) | | | 1 | 0 | 1 (100.0%) | 0 |
| | C) MS 60 mg/NTX 0.01 mg | 20 | 0 | | | 0 | 0 | 0 | 0 |
| | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |
| HYPER- | A) PLACEBO | 22 | 0 | Treatment | 0.838 | 0 | 0 | 0 | 0 |
| TONLA | B) MS 60 mg | 23 | 1 (4.3%) | | | 1 | 0 | 1 (100.0%) | 0 |
| | C) MS 60 mg/NTX 0.01 mg | 20 | 1 (5.0%) | | | 1 | 0 | 0 | 1 (100.0%) |
| | D) MS 60 mg/NTX 0.1 mg | 20 | 1 (5.0%) | | | 1 | 0 | 1 (100.0%) | 0 |
| | E) MS 60 mg/NTX 1 mg | 20 | 0 | | | 0 | 0 | 0 | 0 |
| PARES- | A) PLACEBO | 22 | 0 | Treatment | 0.549 | 0 | 0 | 0 | 0 |
| THESIA | B) MS 60 mg | 23 | 0 | | | 0 | 0 | 0 | 0 |
| | C) MS 60 mg/NTX 0.01 mg | 20 | 1 (5.0%) | | | 1 | 1 (100.0%) | 0 | 0 |
| | D) MS 60 mg/NTX 0.1 mg | 20 | 1 (5.0%) | | | 1 | 0 | 1 (100.0%) | 0 |
| | E) MS 60 mg/NTX 1 mg | 20 | 1 (5.0%) | | | 1 | 0 | 1 (100.0%) | 0 |
| SOM- | A) PLACEBO | 22 | 0 | Treatment | 0.021* | 0 | 0 | 0 | 0 |
| NOLENCE | B) MS 60 mg | 23 | 3 (13.0%) | A-E | 0.018* | 3 | 2 (66.7%) | 1 (33.3%) | 0 |
| | C) MS 60 mg/NTX 0.01 mg | 20 | 0 | C-E | 0.047* | 0 | 0 | 0 | 0 |
| | D) MS 60 mg/NTX 0.1 mg | 20 | 3 (15.0%) | | | 3 | 0 | 2 (66.7%) | 1 (33.3%) |
| | E) MS 60 mg/NTX 1 mg | 20 | 5 (25.0%) | | | 5 | 4 (80.0%) | 1 (20.0%) | 0 |
| TREMOR | A) PLACEBO | 22 | 0 | Treatment | 0.571 | 0 | 0 | 0 | 0 |
| | B) MS 60 mg | 23 | 0 | | | 0 | 0 | 0 | 0 |
| | C) MS 60 mg/NTX 0.01 mg | 20 | 0 | | | 0 | 0 | 0 | 0 |
| | D) MS 60 mg/NTX 0.1 mg | 20 | 1 (5.0%) | | | 1 | 1 (100.0%) | 0 | 0 |
| | E) MS 60 mg/NTX 1 mg | 20 | 0 | | | 0 | 0 | 0 | 0 |

RESPIRATORY

| | | | | | | | | | |
|---|---|---|---|---|---|---|---|---|---|
| ALL EVENTS | A) PLACEBO | 22 | 1 (4.5%) | Treatment | 0.654 | 1 | 1 (100.0%) | 0 | 0 |
| | B) MS 60 mg | 23 | 0 | | | 0 | 0 | 0 | 0 |
| | C) MS 60 mg/NTX 0.01 mg | 20 | 1 (5.0%) | | | 1 | 0 | 1 (100.0%) | 0 |
| | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |
| EPISTAXIS | A) PLACEBO | 22 | 0 | Treatment | 0.571 | 0 | 0 | 0 | 0 |
| | B) MS 60 mg | 23 | 0 | | | 0 | 0 | 0 | 0 |
| | C) MS 60 mg/NTX 0.01 mg | 20 | 1 (5.0%) | | | 1 | 0 | 1 (100.0%) | 0 |
| | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |
| RHINITIS | A) PLACEBO | 22 | 1 (4.5%) | Treatment | 0.780 | 1 | 1 (100.0%) | 0 | 0 |
| | B) MS 60 mg | 23 | 0 | | | 0 | 0 | 0 | 0 |
| | C) MS 60 mg/NTX 0.01 mg | 20 | 0 | | | 0 | 0 | 0 | 0 |
| | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |

SKIN/APPENDAGES

| | | | | | | | | | |
|---|---|---|---|---|---|---|---|---|---|
| ALL EVENTS | A) PLACEBO | 22 | 0 | Treatment | 0.211 | 0 | 0 | 0 | 0 |
| | B) MS 60 mg | 23 | 1 (4.3%) | | | 1 | 1 (100.0%) | 0 | 0 |
| | C) MS 60 mg/NTX 0.01 mg | 20 | 3 (15.0%) | | | 4 | 1 (25.0%) | 3 (75.0%) | 0 |
| | D) MS 60 mg/NTX 0.1 mg | 20 | 3 (15.0%) | | | 3 | 0 | 3 (100.0%) | 0 |
| | E) MS 60 mg/NTX 1 mg | 20 | 3 (15.0%) | | | 4 | 3 (75.0%) | 1 (25.0%) | 0 |
| PURITUS | A) PLACEBO | 22 | 0 | Treatment | 0.081 | 0 | 0 | 0 | 0 |
| | B) MS 60 mg | 23 | 0 | | | 0 | 0 | 0 | 0 |
| | C) MS 60 mg/NTX 0.01 mg | 20 | 3 (15.0%) | | | 3 | 1 (33.3%) | 2 (66.7%) | 0 |
| | D) MS 60 mg/NTX 0.1 mg | 20 | 3 (15.0%) | | | 3 | 0 | 3 (100.0%) | 0 |
| | E) MS 60 mg/NTX 1 mg | 20 | 2 (10.0%) | | | 2 | 2 (100.0%) | 0 | 0 |
| RASH | A) PLACEBO | 22 | 0 | Treatment | 0.412 | 0 | 0 | 0 | 0 |
| | B) MS 60 mg | 23 | 0 | | | 0 | 0 | 0 | 0 |
| | C) MS 60 mg/NTX 0.01 mg | 20 | 1 (5.0%) | | | 1 | 0 | 1 (100.0%) | 0 |
| | D) MS 60 mg/NTX 0.1 mg | 20 | 0 | | | 0 | 0 | 0 | 0 |
| | E) MS 60 mg/NTX 1 mg | 20 | 1 (5.0%) | | | 1 | 0 | 1 (100.0%) | 0 |
| SWEATING | A) PLACEBO | 22 | 0 | Treatment | 0.907 | 0 | 0 | 0 | 0 |
| | B) MS 60 mg | 23 | 1 (4.3%) | | | 1 | 1 (100.0%) | 0 | 0 |
| | C) MS 60 mg/NTX 0.01 mg | 20 | 0 | | | 0 | 0 | 0 | 0 |
| | D) MS 60 mg/NTX 0.1 mg | 20 | 0 | | | 0 | 0 | 0 | 0 |
| | E) MS 60 mg/NTX 1 mg | 20 | 1 (5.0%) | | | 1 | 1 (100.0%) | 0 | 0 |

SPECIAL SENSES

| | | | | | | | | | |
|---|---|---|---|---|---|---|---|---|---|
| ALL EVENTS | A) PLACEBO | 22 | 0 | Treatment | 0.201 | 0 | 0 | 0 | 0 |
| | B) MS 60 mg | 23 | 2 (8.7%) | | | 2 | 2 (100.0%) | 0 | 0 |
| | C) MS 60 mg/NTX 0.01 mg | 20 | 2 (10.0%) | | | 2 | 2 (100.0%) | 0 | 0 |
| | D) MS 60 mg/NTX 0.1 mg | 20 | 3 (15.0%) | | | 3 | 2 (66.7%) | 1 (33.3%) | 0 |
| | E) MS 60 mg/NTX 1 mg | 20 | 0 | | | 0 | 0 | 0 | 0 |
| CONJUNC- | A) PLACEBO | 22 | 0 | Treatment | 0.201 | 0 | 0 | 0 | 0 |
| TIVITIS | B) MS 60 mg | 23 | 2 (8.7%) | | | 2 | 2 (100.0%) | 0 | 0 |
| | C) MS 60 mg/NTX 0.01 mg | 20 | 2 (10.0%) | | | 2 | 2 (100.0%) | 0 | 0 |
| | D) MS 60 mg/NTX 0.1 mg | 20 | 3 (15.0%) | | | 3 | 2 (66.7%) | 0 | 0 |
| | E) MS 60 mg/NTX 1 mg | 20 | 0 | | | 0 | 0 | 0 | 0 |

UROGENITAL

| | | | | | | | | | |
|---|---|---|---|---|---|---|---|---|---|
| ALL EVENTS | A) PLACEBO | 22 | 0 | Treatment | 0.907 | 0 | 0 | 0 | 0 |
| | B) MS 60 mg | 23 | 1 (4.3%) | | | 1 | 1 (100.0%) | 0 | 0 |
| | C) MS 60 mg/NTX 0.01 mg | 20 | 0 | | | 0 | 0 | 0 | 0 |
| | D) MS 60 mg/NTX 0.1 mg | 20 | 1 (5.0%) | | | 1 | 1 (100.0%) | 0 | 0 |
| | E) MS 60 mg/NTX 1 mg | 20 | 0 | | | 0 | 0 | 0 | 0 |
| METROR- | A) PLACEBO | 22 | 0 | Treatment | 1.000 | 0 | 0 | 0 | 0 |
| RHAGIA | B) MS 60 mg | 23 | 1 (4.3%) | | | 1 | 1 (100.0%) | 0 | 0 |
| | C) MS 60 mg/NTX 0.01 mg | 20 | 0 | | | 0 | 0 | 0 | 0 |
| | D) MS 60 mg/NTX 0.1 mg | 20 | 0 | | | 0 | 0 | 0 | 0 |
| | E) MS 60 mg/NTX 1 mg | 20 | 0 | | | 0 | 0 | 0 | 0 |
| URINARY | A) PLACEBO | 22 | 0 | Treatment | 0.571 | 0 | 0 | 0 | 0 |
| RETENTION | B) MS 60 mg | 23 | 0 | | | 0 | 0 | 0 | 0 |
| | C) MS 60 mg/NTX 0.01 mg | 20 | 0 | | | 0 | 0 | 0 | 0 |
| | D) MS 60 mg/NTX 0.1 mg | 20 | 1 (5.0%) | | | 1 | 1 (100.0%) | 0 | 0 |
| | E) MS 60 mg/NTX 1 mg | 20 | 0 | | | 0 | 0 | 0 | 0 |

NOTE:
ADVERSE EVENTS RELATED TO STUDY DRUG ARE DEFINED AS THOSE EVENTS WITH RELATIONSHIP TO STUDY
DRUG “SUSPECTED” OR “PROBABLE”
[1] P-VALUES ARE FROM FISCHER'S EXACT TEST AND ARE PROVIDED FOR OVERALL TREATMENT EFFECT AND SIGNIFICANT PAIRWISE COMPARISONS ONLY
[2] THE DENOMINATOR FOR THE PERCENTAGES IS THE TOTAL NUMBER OF EVENTS.
*, **, ***P-VALUE <=0.05, <=0.01, OR <=<0.001 RESPECTIVELY.

TABLE 26B
SELECTED ADVERSE EVENTS
SAFETY POPULATION, FEMALE PATIENTS

| | | Total | No. Of | | | Number | | | |
|---|---|---|---|---|---|---|---|---|---|
| Adverse Event | | No. Of | Subjects | | P-Value | Of | Severity [2] | | |
| (English) | Treatment | Subjects | W/Event | Source | [1] | Events | Mild | Moderate | Severe |
| DIZZINESS | A) PLACEBO | 22 | 1 (4.5%) | Treatment | 0.022* | 1 | 1 (100.0%) | 0 | 0 |
| | B) MS 60 mg | 23 | 7 (30.4%) | A-B | 0.046* | 9 | 5 (55.6%) | 3 (33.3%) | 1 (11.1%) |
| | C) MS 60 mg/NTX 0.01 mg | 20 | 8 (40.0%) | A-C | 0.007** | 8 | 3 (37.5%) | 4 (50.0%) | 1 (12.5%) |
| | D) MS 60 mg/NTX 0.1 mg | 20 | 9 (45.0%) | A-D | 0.003** | 12 | 5 (41.7%) | 4 (33.3%) | 3 (25.0%) |
| | E) MS 60 mg/NTX 1 mg | 20 | 6 (30.0%) | A-E | 0.040* | 6 | 4 (66.7%) | 2 (33.3%) | 0 |
| NAUSEA | A) PLACEBO | 22 | 3 (13.6%) | Treatment | <0.001*** | 3 | 0 | 1 (33.3%) | 2 (66.7%) |
| | B) MS 60 mg | 23 | 15 (65.2%) | A-B | <0.001*** | 16 | 4 (25.0%) | 10 (62.5%) | 2 (12.5%) |
| | C) MS 60 mg/NTX 0.01 mg | 20 | 15 (75.0%) | A-C | <0.001*** | 16 | 4 (25.0%) | 11 (68.8%) | 1 (6.3%) |
| | D) MS 60 mg/NTX 0.1 mg | 20 | 16 (80.0%) | A-D | <0.001*** | 17 | 5 (29.4%) | 6 (35.3%) | 6 (35.3%) |
| | E) MS 60 mg/NTX 1 mg | 20 | 10 (50.0%) | A-E | 0.018* | 11 | 4 (36.4%) | 5 (45.5%) | 2 (18.2%) |
| SOMNOLENCE | A) PLACEBO | 22 | 0 | Treatment | 0.021* | 0 | 0 | 0 | 0 |
| | B) MS 60 mg | 23 | 3 (13.0%) | A-E | 0.018* | 3 | 2 (66.7%) | 1 (33.3%) | 0 |
| | C) MS 60 mg/NTX 0.01 mg | 20 | 0 | C-E | 0.047* | 0 | 0 | 0 | 0 |
| | D) MS 60 mg/NTX 0.1 mg | 20 | 3 (15.0%) | | | 3 | 0 | 2 (66.7%) | 1 (33.3%) |
| | E) MS 60 mg/NTX 1 mg | 20 | 5 (25.0%) | | | 5 | 4 (80.0%) | 1 (20.0%) | 0 |
| VOMITING | A) PLACEBO | 22 | 3 (13.6%) | Treatment | <0.001*** | 3 | 0 | 0 | 3 (100.0%) |
| | B) MS 60 mg | 23 | 14 (60.9%) | A-B | <0.001** | 14 | 0 | 0 | 14 (100.0%) |
| | C) MS 60 mg/NTX 0.01 mg | 20 | 15 (75.0%) | A-C | <0.001*** | 15 | 0 | 0 | 15 (100.0%) |
| | D) MS 60 mg/NTX 0.1 mg | 20 | 14 (70.0%) | A-D | <0.001*** | 14 | 0 | 0 | 14 (100.0%) |
| | E) MS 60 mg/NTX 1 mg | 20 | 6 (30.0%) | C-E | 0.010* | 6 | 0 | 0 | 6 (100.0%) |
| | | | | D-E | 0.025* | | | | |

NOTE:
ADVERSE EVENTS RELATED TO STUDY DRUG ARE DEFINED AS THOSE EVENTS WITH RELATIONSHIP TO STUDY DRUG “SUSPECTED” OR “PROBABLE”
[1] P-VALUES ARE FROM FISCHER'S EXACT TEST AND ARE PROVIDED FOR OVERALL TREATMENT EFFECT AND SIGNIFICANT PAIRWISE COMPARISONS ONLY
[2] THE DENOMINATOR FOR THE PERCENTAGES IS THE TOTAL NUMBER OF EVENTS.
*, **, ***P-VALUE <=0.05, <=0.01, OR <=<0.001 RESPECTIVELY.

TABLE 26C
ADVERSE EVENTS BY BODY SYSTEM AND SEVERITY
SAFETY POPULATION, MALE PATIENTS

| BODY | | | | | | | | | |
|---|---|---|---|---|---|---|---|---|---|
| SYSTEM | | | | | | | | | |
| ADVERSE | | TOTAL | | | | Num- | | | |
| EVENTS | | NO. OF | NO. OF | | | ber | | | |
| (COSTART | | SUB- | SUBJECTS | | | Of E- | Severity [2] | | |
| ENGLISH) | TREATMENT | JECTS | W/EVENT | SOURCE | P-Value [1] | vents | MILD | Moderate | SEVERE |

TOTAL NUMBER OF EVENTS
ADVERSE EVENTS
(ALL BODY SYSTEMS)

| | | | | | | | | | |
|---|---|---|---|---|---|---|---|---|---|
| ALL EVENTS | A) PLACEBO | 18 | 4 (22.2%) | Treatment | <0.001*** | 5 | 3 (60.0%) | 2 (40.0%) | 0 |
| | B) MS 60 mg | 18 | 13 (72.2%) | A-B | 0.006** | 27 | 10 (37.0%) | 12 (44.4%) | 5 (18.5%) |
| | C) MS 60 mg/NTX 0.01 mg | 21 | 17 (81.0%) | A-C | <0.001*** | 35 | 9 (25.7%) | 16 (45.7%) | 10 (28.6% |
| | D) MS 60 mg/NTX 0.1 mg | 21 | 17 (81.0%) | A-D | <0.001*** | 34 | 11 (32.4%) | 15 (44.1%) | 8 (23.5%) |
| | E) MS 60 mg/NTX 1 mg | 21 | 14 (66.7%) | A-E | 0.009** | 30 | 15 (50.0%) | 12 (40.0%) | 3 (10.0%) |

BODY AS A WHOLE

| | | | | | | | | | |
|---|---|---|---|---|---|---|---|---|---|
| ALL EVENTS | A) PLACEBO | 18 | 1 (5.6%) | Treatment | 0.624 | 1 | 0 | 1 (100.0%) | 0 |
| | B) MS 60 mg | 18 | 2 (11.1%) | | | 2 | 2 (100.0%) | 0 | 0 |
| | C) MS 60 mg/NTX 0.01 mg | 21 | 5 (23.8%) | | | 5 | 1 (20.0%) | 3 (60.0%) | 1 (20.0%) |
| | D) MS 60 mg/NTX 0.1 mg | 21 | 3 (14.3%) | | | 3 | 2 (66.7%) | 1 (33.3%) | 0 |
| | E) MS 60 mg/NTX 1 mg | 21 | 4 (19.0%) | | | 4 | 2 (50.0%) | 2 (50.0%) | 0 |
| ABDOMINAL | A) PLACEBO | 18 | 0 | Treatment | 1.000 | 0 | 0 | 0 | 0 |
| PAIN | B) MS 60 mg | 18 | 0 | | | 0 | 0 | 0 | 0 |
| | C) MS 60 mg/NTX 0.01 mg | 21 | 1 (4.8%) | | | 1 | 0 | 0 | 1 (100.0%) |
| | D) MS 60 mg/NTX 0.1 mg | 21 | 0 | | | 0 | 0 | 0 | 0 |
| | E) MS 60 mg/NTX 1 mg | 21 | 0 | | | 0 | 0 | 0 | 0 |
| ASTHENIA | A) PLACEBO | 18 | 0 | Treatment | 0.940 | 0 | 0 | 0 | 0 |
| | B) MS 60 mg | 18 | 1 (5.6%) | | | 1 | 1 (100.0%) | 0 | 0 |
| | C) MS 60 mg/NTX 0.01 mg | 21 | 1 (4.8%) | | | 1 | 0 | 1 (100.0%) | 0 |
| | D) MS 60 mg/NTX 0.1 mg | 21 | 0 | | | 0 | 0 | 0 | 0 |
| | E) MS 60 mg/NTX 1 mg | 21 | 1 (4.8%) | | | 1 | 1 (100.0%) | 0 | 0 |
| FEVER | A) PLACEBO | 18 | 1 (5.6%) | Treatment | 0.363 | 1 | 0 | 1 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 0 | | | 0 | 0 | 0 | 0 |
| HEADACHE | A) PLACEBO | 18 | 0 | Treatment | 0.637 | 0 | 0 | 0 | 0 |
| | B) MS 60 mg | 18 | 1 (5.6%) | | | 1 | 1 (100.0%) | 0 | 0 |
| | C) MS 60 mg/NTX 0.01 mg | 21 | 2 (9.5%) | | | 2 | 1 (50.0%) | 1 (50.0%) | 0 |
| | D) MS 60 mg/NTX 0.1 mg | 21 | 1 (4.8%) | | | 1 | 1 (100.0%) | 0 | 0 |
| | E) MS 60 mg/NTX 1 mg | 21 | 3 (14..3%) | | | 3 | 1 (33.0%) | 2 (66.7%) | 0 |
| OVERDOSE | A) PLACEBO | 18 | 0 | Treatment | 1.000 | 0 | 0 | 0 | 0 |
| | B) MS 60 mg | 18 | 0 | | | 0 | 0 | 0 | 0 |
| | C) MS 60 mg/NTX 0.01 mg | 21 | 1 (4.8%) | | | 0 | 0 | 1 (100.0%) | 0 |
| | D) MS 60 mg/NTX 0.1 mg | 21 | 0 | | | 0 | 0 | 0 | 0 |
| | E) MS 60 mg/NTX 1 mg | 21 | 0 | | | 0 | 0 | 0 | 0 |
| PAIN | A) PLACEBO | 18 | 0 | Treatment | 0.192 | 0 | 0 | 0 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 2 (9.5%) | | | 2 | 1 (50.0%) | 1 (50.0%) | 0 |
| | E) MS 60 mg/NTX 1 mg | 21 | 0 | | | 0 | 0 | 0 | 0 |

CARDIOVASCULAR

| | | | | | | | | | |
|---|---|---|---|---|---|---|---|---|---|
| ALL EVENTS | A) PLACEBO | 18 | 0 | Treatment | 0.540 | 0 | 0 | 0 | 0 |
| | B) MS 60 mg | 18 | 1 (5.6%) | | | 1 | 1 (100.0%) | 0 | 0 |
| | C) MS 60 mg/NTX 0.01 mg | 21 | 1 (4.8%) | | | 1 | 1 (100.0%) | 0 | 0 |
| | D) MS 60 mg/NTX 0.1 mg | 21 | 3 (14.3%) | | | 3 | 1 (33.3%) | 2 (66.7%) | 0 |
| | E) MS 60 mg/NTX 1 mg | 21 | 1 (4.8%) | | | 1 | 1 (100.0%) | 0 | 0 |
| HEMOR- | A) PLACEBO | 18 | 0 | Treatment | 1.000 | 0 | 0 | 0 | 0 |
| RHAGE | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 1 (4.8%) | | | 1 | 0 | 1 (100.0%) | 0 |
| | E) MS 60 mg/NTX 1 mg | 21 | 0 | | | 0 | 0 | 0 | 0 |
| HYPER- | A) PLACEBO | 18 | 0 | Treatment | 1.000 | 0 | 0 | 0 | 0 |
| TENSION | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 1 (4.8%) | | | 1 | 1 (100.0%) | 0 | 0 |
| | E) MS 60 mg/NTX 1 mg | 21 | 0 | | | 0 | 0 | 0 | 0 |
| VASO- | A) PLACEBO | 18 | 0 | Treatment | 1.000 | 0 | 0 | 0 | 0 |
| DILATATION | B) MS 60 mg | 18 | 1 (5.6%) | | | 1 | 1 (100.0%) | 0 | 0 |
| | C) MS 60 mg/NTX 0.01 mg | 21 | 1 (4.8%) | | | 1 | 1 (100.0%) | 0 | 0 |
| | D) MS 60 mg/NTX 0.1 mg | 21 | 1 (4.8%) | | | 1 | 0 | 1 (100.0%) | 0 |
| | E) MS 60 mg/NTX 1 mg | 21 | 1 (4.8%) | | | 1 | 1 (100.0%) | 0 | 0 |

DIGESTIVE

| | | | | | | | | | |
|---|---|---|---|---|---|---|---|---|---|
| ALL EVENTS | A) PLACEBO | 18 | 1 (5.6%) | Treatment | 0.017* | 1 | 0 | 1 (100.0%) | 0 |
| | B) MS 60 mg | 18 | 7 (38.9%) | A-B | 0.040* | 10 | 2 (20.0%) | 4 (40.0%) | 4 (40.0%) |
| | C) MS 60 mg/NTX 0.01 mg | 21 | 8 (38.1%) | A-C | 0.023* | 15 | 3 (20.0%) | 4 9 26.7%) | 8 (53.3%) |
| | D) MS 60 mg/NTX 0.1 mg | 21 | 11 (52.4%) | A-D | <0.001** | 14 | 2 (14.3%) | 5 (35.7%) | 7 (50.0%) |
| | E) MS 60 mg/NTX 1 mg | 21 | 5 (23.8%) | | | 7 | 1 (14.3%) | 3 (42.9%) | 3 (42.9%0 |
| NAUSEA | A) PLACEBO | 18 | 1 (5.6%) | Treatment | 0.048* | 1 | 0 | 1 (100.0%) | 0 |
| | B) MS 60 mg | 18 | 6 (33.3%) | | 0.023* | 6 | 2 (33.3%) | 4 (66.7%) | 0 |
| | C) MS 60 mg/NTX 0.01 mg | 21 | 8 (38.1%) | | 0.010* | 10 | 3 (30.0%) | 4 (40.0%) | 3 (30.0%) |
| | D) MS 60 mg/NTX 0.1 mg | 21 | 9 (42.9%) | | | 9 | 2 (22.2%) | 5 (55.6%) | 2 (20.2%) |
| | E) MS 60 mg/NTX 1 mg | 21 | 4 (19.0%) | | | 4 | 1 (25.0%) | 3 (75.0%) | 0 |
| VOMITING | A) PLACEBO | 18 | 0 | Treatment | 0.166 | 0 | 0 | 0 | 0 |
| | B) MS 60 mg | 18 | 4 (22.2%) | A-C | | 4 | 0 | 0 | 4 (100.0%) |
| | C) MS 60 mg/NTX 0.01 mg | 21 | 5 (23.8%) | A-D | | 5 | 0 | 0 | 5 (100.0%) |
| | D) MS 60 mg/NTX 0.1 mg | 21 | 5 (23.8%) | | | 5 | 0 | 0 | 5 (100.0%) |
| | E) MS 60 mg/NTX 1 mg | 21 | 3 (14.3%) | | | 3 | 0 | 0 | 3 (100.0%) |

MUSCULOSKELETAL

| | | | | | | | | | |
|---|---|---|---|---|---|---|---|---|---|
| ALL EVENTS | A) PLACEBO | 18 | 0 | Treatment | 0.363 | 0 | 0 | 0 | 0 |
| | B) MS 60 mg | 18 | 1 (5.6%) | | | 1 | 0 | 1 (100.0%) | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 0 | | | 0 | 0 | 0 | 0 |
| MYALGIA | A) PLACEBO | 18 | 0 | Treatment | 0.363 | 0 | 0 | 0 | 0 |
| | B) MS 60 mg | 18 | 1 (5.6%) | | | 1 | 0 | 1 (100.0%) | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 0 | | | 0 | 0 | 0 | 0 |

NERVOUS SYSTEM

| | | | | | | | | | |
|---|---|---|---|---|---|---|---|---|---|
| ALL EVENTS | A) PLACEBO | 18 | 1 (5.6%) | Treatment | 0.016* | 1 | 1 (100.0%) | 0 | 0 |
| | B) MS 60 mg | 18 | 8 (44.4%) | A-B | 0.017* | 10 | 4 (40.0%) | 5 (50.0%) | 1 (10.0%) |
| | C) MS 60 mg/NTX 0.01 mg | 21 | 10 (47.6%) | A-C | 0.004** | 11 | 2 (18.2%) | 8 72.7%) | 1 (9.1%) |
| | D) MS 60 mg/NTX 0.1 mg | 21 | 10 (47.6%) | A-D | 0.004** | 10 | 3 (30.0%) | 6 (60.0%) | 1 (10.0%) |
| | E) MS 60 mg/NTX 1 mg | 21 | 10 (47.6%) | A-E | 0.004** | 14 | 8 (57.1%) | 6 (42.9%) | 0 |
| ANXIETY | A) PLACEBO | 18 | 0 | Treatment | 1.000 | 0 | 0 | 0 | 0 |
| | B) MS 60 mg | 18 | 0 | | | 0 | 0 | 0 | 0 |
| | C) MS 60 mg/NTX 0.01 mg | 21 | 1 (4.8%) | | | 1 | 0 | 1 (100.0%) | 0 |
| | D) MS 60 mg/NTX 0.1 mg | 21 | 1 (4.8%) | | | 1 | 0 | 1 (100.0%) | 0 |
| | E) MS 60 mg/NTX 1 mg | 21 | 0 | | | 0 | 0 | 0 | 0 |
| DIZZINESS | A) PLACEBO | 18 | 1 (5.6%) | Treatment | 0.065 | 1 | 1 (100.0%) | 0 | 0 |
| | B) MS 60 mg | 18 | 8 (44.4%) | A-B | 0.017* | 8 | 4 (50.0%) | 3 (37.5%) | 1 (12.5%) |
| | C) MS 60 mg/NTX 0.01 mg | 21 | 8 (38.1%) | A-C | 0.023* | 8 | 2 (25.0%) | 5 (62.5%) | 1 (12.5%) |
| | D) MS 60 mg/NTX 0.1 mg | 21 | 8 (38.1%) | A-D | 0.023* | 8 | 1 (12.5%) | 6 (75.0%) | 1 (12.5%) |
| | E) MS 60 mg/NTX 1 mg | 21 | 7 (33.3%) | A-E | 0.048* | 7 | 4 (57.1%) | 3 (42.9%) | 0 |
| DRY MOUTH | A) PLACEBO | 18 | 0 | Treatment | 0.192 | 0 | 0 | 0 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 2 (9.5%) | | | 2 | 1 (50.0%) | 1 (50.0%) | 0 |
| EUPHORIA | A) PLACEBO | 18 | 0 | Treatment | 1.000 | 0 | 0 | 0 | 0 |
| | B) MS 60 mg | 18 | 0 | | | 0 | 0 | 0 | 0 |
| | C) MS 60 mg/NTX 0.01 mg | 21 | 1 (4.8%) | | | 1 | 0 | 1 (100.0%) | 0 |
| | D) MS 60 mg/NTX 0.1 mg | 21 | 1 (4.8%) | | | 1 | 1 (100.0%) | 0 | 0 |
| | E) ms 60 mg/NTX 1 mg | 21 | 0 | | | 0 | 0 | 0 | 0 |
| PARES- | A) PLACEBO | 18 | 0 | Treatment | 1.000 | 0 | 0 | 0 | 0 |
| THESIA | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 1 (4.8%) | | | 1 | 1 (100.0%) | 0 | 0 |
| SOM- | A) PLACEBO | 18 | 0 | Treatment | 0.265 | 0 | 0 | 0 | 0 |
| NOLENCE | B) MS 60 mg | 18 | 1 (5.6%) | | | 1 | 0 | 1 (100.0%) | 0 |
| | C) MS 60 mg/NTX 0.01 mg | 21 | 1 (4.8%) | | | 1 | 0 | 1 (100.0%) | 0 |
| | D) MS 60 mg/NTX 0.1 mg | 21 | 0 | | | 0 | 0 | 0 | 0 |
| | E) MS 60 mg/NTX 1 mg | 21 | 3 (14.3%) | | | 3 | 1 (33.3%) | 2 (66.7%) | 0 |
| TREMOR | A) PLACEBO | 18 | 0 | Treatment | 0.727 | 0 | 0 | 0 | 0 |
| | B) MS 60 mg | 18 | 1 (5.6%) | | | 1 | 0 | 1 (100.0%) | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 1 (4.8%) | | | 1 | 1 (100.0%) | 0 | 0 |

RESPIRATORY

| | | | | | | | | | |
|---|---|---|---|---|---|---|---|---|---|
| ALL EVENTS | A) PLACEBO | 18 | 1 (5.6%) | Treatment | 0.727 | 1 | 1 (100.0) | 0 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 1 (4.8%) | | | 1 | 1 (100.0) | 0 | 0 |
| DYSPNEA | A) PLACEBO | 18 | 0 | Treatment | 1.000 | 0 | 0 | 0 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 1 (4.8%) | | | 1 | 1 (100.0%) | 0 | 0 |
| EPISTAXIS | A) PLACEBO | 18 | 1 (5.6%) | Treatment | 0.363 | 1 | 1 (100.0%) | 0 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | | | | 0 | 0 | 0 | 0 |

SKIN/APPENDAGES

| | | | | | | | | | |
|---|---|---|---|---|---|---|---|---|---|
| ALL EVENTS | A) PLACEBO | 18 | 0 | Treatment | 0.399 | 0 | 0 | 0 | 0 |
| | B) MS 60 mg | 18 | 3 (16.7%) | | | 3 | 1 (33.3%) | 2 (66.7%) | 0 |
| | C) MS 60 mg/NTX 0.01 mg | 21 | 1 (4.8%) | | | 1 | 1 (100.0%) | 0 | 0 |
| | D) MS 60 mg/NTX 0.1 mg | 21 | 1 (4.8%) | | | 1 | 0 | 1 (100.0%) | 0 |
| | E) MS 60 mg/NTX 1 mg | 21 | 1 (4.8%) | | | 1 | 0 | 1 (100.0%) | 0 |
| PURITUS | A) PLACEBO | 18 | 0 | Treatment | 0.416 | 0 | 0 | 0 | 0 |
| | B) MS 60 mg | 18 | 2 (11.1%) | | | 2 | 1 (50.0%) | 1 (50.0%) | 0 |
| | C) MS 60 mg/NTX 0.01 mg | 21 | 1 (4.8%) | | | 1 | 1 (100.0%) | 0 | 0 |
| | D) MS 60 mg/NTX 0.1 mg | 21 | 1 (4.8%) | | | 1 | 0 | 1 (100.0%) | 0 |
| | E) MS 60 mg/NTX 1 mg | 21 | 0 | | | 0 | 0 | 0 | 0 |
| SWEATING | A) PLACEBO | 18 | 0 | Treatment | 0.727 | 0 | 0 | 0 | 0 |
| | B) MS 60 mg | 18 | 1 (5.6%) | | | 1 | 0 | 1 (100.0%) | 0 |
| | C) MS 60 mg/NTX 0.01 mg | 21 | 0 | | | 0 | 0 | 0 | 0 |
| | D) MS 60 mg/NTX 0.1 mg | 21 | 0 | | | 0 | 0 | 0 | 0 |
| | E) MS 60 mg/NTX 1 mg | 21 | 1 (4.8%) | | | 1 | 0 | 1 (100.0%) | 0 |

SPECIAL SENSES

| | | | | | | | | | |
|---|---|---|---|---|---|---|---|---|---|
| ALL EVENTS | A) PLACEBO | 18 | 1 (5.6%) | Treatment | 0.958 | 1 | 1 (100.0%) | 0 | 0 |
| | B) MS 60 mg | 18 | 0 | | | 0 | 0 | 0 | 0 |
| | C) MS 60 mg/NTX 0.01 mg | 21 | 1 (4.8%) | | | 1 | 1 (100.0%) | 0 | 0 |
| | D) MS 60 mg/NTX 0.1 mg | 21 | 1 (4.8%) | | | 1 | 1 (100.0%) | 0 | 0 |
| | E) MS 60 mg/NTX 1 mg | 21 | 2 (9.5%) | | | 2 | 2 (100.0%) | 0 | 0 |
| CONJUNC- | A) PLACEBO | 18 | 1 (5.6%) | Treatment | 0.958 | 1 | 1 (100.0%) | 0 | 0 |
| TIVITIS | B) MS 60 mg | 18 | 0 | | | 0 | 0 | 0 | 0 |
| | C) MS 60 mg/NTX 0.01 mg | 21 | 1 (4.8%) | | | 1 | 1 (100.0%) | 0 | 0 |
| | D) MS 60 mg/NTX 0.1 mg | 21 | 1 (4.8%) | | | 1 | 1 (100.0%) | 0 | 0 |
| | E) MS 60 mg/NTX 1 mg | 21 | 2 (9.5%) | | | 2 | 2 (100.0%) | 0 | 0 |

UROGENITAL

| | | | | | | | | | |
|---|---|---|---|---|---|---|---|---|---|
| ALL EVENTS | A) PLACEBO | 18 | 0 | Treatment | 0.507 | 0 | 0 | 0 | 0 |
| | B) MS 60 mg | 18 | 0 | | | 0 | 0 | 0 | 0 |
| | C) MS 60 mg/NTX 0.01 mg | 21 | 1 (4.8%) | | | 1 | 0 | 1 (100.0%) | 0 |
| | D) MS 60 mg/NTX 0.1 mg | 21 | 2 (9.5%) | | | 2 | 2 (100.0%) | 0 | 0 |
| | E) MS 60 mg/NTX 1 mg | 21 | 0 | | | 0 | 0 | 0 | 0 |
| DYSURIA | A) PLACEBO | 18 | 0 | Treatment | 1.000 | 0 | 0 | 0 | 0 |
| | B) MS 60 mg | 18 | 0 | | | 0 | 0 | 0 | 0 |
| | C) MS 60 mg/NTX 0.01 mg | 21 | 0 | | | 0 | 0 | 0 | 0 |
| | D) MS 60 mg/NTX 0.1 mg | 21 | 1 (4.8%) | | | 1 | 1 (100.0%) | 0 | 0 |
| | E) MS 60 mg/NTX 1 mg | 21 | 0 | | | 0 | 0 | 0 | 0 |
| URINARY | A) PLACEBO | 18 | 0 | Treatment | 1.000 | 0 | 0 | 0 | 0 |
| RETENTION | B) MS 60 mg | 18 | 0 | | | 0 | 0 | 0 | 0 |
| | C) MS 60 mg/NTX 0.01 mg | 21 | 1 (4.8%) | | | 1 | 0 | 1 (100.0%) | 0 |
| | D) MS 60 mg/NTX 0.1 mg | 21 | 1 (4.8%) | | | 1 | 1 (100.0%) | 0 | 0 |
| | E) MS 60 mg/NTX 1 mg | 21 | 0 | | | 0 | 0 | 0 | 0 |

NOTE:
ADVERSE EVENTS RELATED TO STUDY DRUG ARE DEFINED AS THOSE EVENTS WITH RELATIONSHIP TO STUDY DRUG “SUSPECTED” OR “PROBABLE.”
[1] P-VALUES ARE FROM FISHER'S EXACT TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
*, **, ***P-VALUE <=0.05, <=0.01, OR <=<0.001 RESPECTIVELY.

TABLE 26D
SELECTED ADVERSE EVENTS
SAFETY POPULATION, MALE PATIENTS

| ADVERSE | | TOTAL | NO. OF | | | NUMBER | | | |
|---|---|---|---|---|---|---|---|---|---|
| EVENT | | NO. OF | SUBJECTS | | P-VALUE | OF | SEVERITY [2] | | |
| (ENGLISH) | TREATMENT | SUBJECTS | W/EVENT | SOURCE | [1] | EVENTS | MILD | MODERATE | SEVERE |
| DIZZINESS | A) PLACEBO | 18 | 1 (5.6%) | Treatment | 0.065 | 1 | 1 (100.0%) | 0 | 0 |
| | B) MS 60 mg | 18 | 8 (44.4%) | A-B | 0.017* | 8 | 4 (50.0%) | 3 (37.5%) | 1 (12.5%) |
| | C) MS 60 mg/NTX | 21 | 8 (38.1%) | A-C | 0.023* | 8 | 2 (25.0%) | 5 (62.5%) | 1 (12.5%) |
| | 0.01 mg | | | | | | | | |
| | D) MS 60 mg/NTX | 21 | 8 (38.1%) | A-D | 0.023* | 8 | 1 (12.5%) | 6 (75.0%) | 1 (12.5%) |
| | 0.1 mg | | | | | | | | |
| | E) MS 60 mg/NTX | 21 | 7 (33.3%) | A-E | 0.048* | 7 | 4 (57.1%) | 3 (42.9%) | 0 |
| | 1 mg | | | | | | | | |
| NAUSEA | A) PLACEBO | 18 | 1 (5.6%) | Treatment | 0.048* | 1 | 0 | 1 (100.0%) | 0 |
| | B) MS 60 mg | 18 | 6 (33.3%) | | 0.023* | 6 | 2 (33.3%) | 4 (66.7%) | 0 |
| | C) MS 60 mg/NTX | 21 | 8 (38.1%) | | 0.010* | 10 | 3 (30.0%) | 4 (40.0%) | 3 (30.0%) |
| | 0.01 mg | | | | | | | | |
| | D) MS 60 mg/NTX | 21 | 9 (42.9%) | | | 9 | 2 (22.2%) | 5 (55.6%) | 2 (20.2%) |
| | 0.1 mg | | | | | | | | |
| | E) MS 60 mg/NTX | 21 | 4 (19.0%) | | | 4 | 1 (25.0%) | 3 (75.0%) | 0 |
| | 1 mg | | | | | | | | |
| SOMNOLENCE | A) PLACEBO | 18 | 0 | Treatment | 0.265 | 0 | 0 | 0 | 0 |
| | B) MS 60 mg | 18 | 1 (5.6%) | | | 1 | 0 | 1 (100.0%) | 0 |
| | C) MS 60 mg/NTX | 21 | 1 (4.8%) | | | 1 | 0 | 1 (100.0%) | 0 |
| | 0.01 mg | | | | | | | | |
| | D) MS 60 mg/NTX | 21 | 0 | | | 0 | 0 | 0 | 0 |
| | 0.1 mg | | | | | | | | |
| | E) MS 60 mg/NTX | 21 | 3 (14.3%) | | | 3 | 1 (33.3%) | 2 (66.7%) | 0 |
| | 1 mg | | | | | | | | |
| VOMITING | A) PLACEBO | 18 | 0 | Treatment | 0.166 | 0 | 0 | 0 | 0 |
| | B) MS 60 mg | 18 | 4 (22.2%) | A-C | | 4 | 0 | 0 | 4 (100.0%) |
| | C) MS 60 mg/NTX | 21 | 5 (23.8%) | A-D | | 5 | 0 | 0 | 5 (100.0%) |
| | 0.01 mg | | | | | | | | |
| | D) MS 60 mg/NTX | 21 | 5 (23.8%) | | | 5 | 0 | 0 | 5 (100.0%) |
| | 0.1 mg | | | | | | | | |
| | E) MS 60 mg/NTX | 21 | 3 (14.3%) | | | 3 | 0 | 0 | 3 (100.0%) |
| | 1 mg | | | | | | | | |

NOTE:
ADVERSE EVENTS RELATED TO STUDY DRUG ARE DEFINED AS THOSE EVENTS WITH RELATIONSHIP TO STUDY DRUG “SUSPECTED” OR “PROBABLE.”
[1] P-VALUES ARE FROM FISHER'S EXACT TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
*, **, ***P-VALUE <=0.05, <=0.01, OR <=<0.001 RESPECTIVELY.

### EXAMPLE 3

An additional clinical study using morphine alone and in combination with low doses of naltrexone was designed substantially the same as that described in Example 1, with the following differences: (1) six treatment groups (not 5) with three different doses of NTX (0.1 mg, 0.01 mg and 0.001 mg) in combination with MS 60 mg versus MS 60 mg alone, versus NTX 0.01 mg alone, and versus placebo alone, in subjects with moderate to severe pain in a postsurgical dental pain clinical study; (2) each group was 50 patients (not 40) for a total of 300 (not 200); (3) subjects had three or four full or partial bony impacted third molars (not 2 or more impacted third molars); (4) meaningful pain relief only (not meaningful and perceptible pain relief with two stopwatches) was measured using one stopwatch; (5) the primary efficacy variables included TOTPAR-4 and SPID-4 measured through 4 hours (not TOTPAR-8 and SPID-8 measured through 8 hours); (6) the secondary efficacy variables included 6 and 8 hour Total Pain Relief Scores (TOTPAR-6 AND TOTPAR-8), 6 and 8 hour Sum of Pain Intensity Difference Scores (SPID-6 and SPID-8), and Time to Onset of Analgesia, time to an hourly PID Score of 1, instead of Time to Onset of First Perceptible Pain Relief; (7) additional exclusion criteria were patients with known history of severe hepatic or renal impairment, and midazolam (Versed) was not permissible medication during surgery; and (8) for adverse events, body systems and preferred terms were from the MedDRA (not the COSTART) dictionary.

A total of 304 subjects were randomized; among them 302 subjects were deemed evaluable (Table 27).

TABLE 27
Analysis Populations, All Patients
Treatments

| | | | | MS (60 mg) | MS (60 mg) | MS (60 mg) | |
|---|---|---|---|---|---|---|---|
| | | | NTX | NTX | NTX | NTX | |
| | Placebo | MS (60 mg) | 0.01 MG | (0.001 mg) | (0.01 mg) | (0.1 mg) | Total |
| Patients Enrolled [1] | 51 | 53 | 51 | 50 | 51 | 48 | 304 |
| Safety | 51 (100.0%) | 53 (100.0%) | 51 (100.0%) | 50 (100.0%) | 51 (100.0%) | 48 (100.0%) | 304 (100.0%) |
| Intent-To-Treat | 51 (100.0%) | 53 (100.0%) | 51 (100.0%) | 50 (100.0%) | 51 (100.0%) | 48 (100.0%) | 304 (100.0%) |
| Evaluable | 51 (100.0%) | 53 (100.0%) | 51 (100.0%) | 49 (98.0%) | 51 (100.0%) | 47 (97.9%) | 302 (99.3%) |

[1] PATIENTS WITH DEMOGRAPHIC INFORMATION.

The demographic and baseline characteristics were summarized by treatment groups for the ITT population (all randomized patients) and the evaluable population (all randomized patients with at least one efficacy evaluation at 90 minutes or more after dosing) (Table 28). Demographic characteristics included age, race/ethnicity, sex, weight, height, medical history, teeth extracted (impacted and non-impacted), baseline pain intensity, and baseline visual analog scale.

The demographics for the total ITT population were generally comparable across all 5 treatment groups. Subjects ranged in age from 16 to 49 years; 66.8% were Caucasian and 53.3% were female. There were some differences among treatment groups in the number of third molars extracted and the degree of impaction of third molar extracted. No adjustments in the analyses were made to take into account differences among treatment groups. These differences had little or no influence on pain assessments at baseline. The baseline pain intensity scores (Table 29A) and visual analog scale scores (Table 29B) were generally comparable across treatment groups.

TABLE 28
Baseline Characteristics
Intent-To-Treat Population, All Patients

| | | | | | | | | MS (60 mg) | | MS (60 mg) | | | | | | |
|---|---|---|---|---|---|---|---|---|---|---|---|---|---|---|---|---|
| | | | | MS | | | | NTX | | NTX | | MS (60 mg) | | | | P-Value |
| | | Placebo | | (60 mg) | | NTX 0.01 mg | | (0.001 mg) | | (0.01 mg) | | NTX (0.1 mg) | | TOTAL | | [1] |
| Age (yrs) | N | 51 | | 53 | | 51 | | 50 | | 51 | | 48 | | 304 | | 0.434 |
| | Mean | 22.5 | | 23.4 | | 24.0 | | 22.5 | | 24.1 | | 24.0 | | 23.4 | | |
| | SD | 3.84 | | 5.85 | | 5.41 | | 4.37 | | 5.97 | | 6.17 | | 5.34 | | |
| | Median | 22.0 | | 22.0 | | 23.0 | | 22.0 | | 22.0 | | 21.5 | | 22.0 | | |
| | Range | 16-31 | | 16-49 | | 16-41 | | 16-38 | | 16-41 | | 17-40 | | 16-49 | | |
| Gender | Male | 19 | (37.3%) | 25 | (47.2%) | 21 | (41.2%) | 32 | (64.0%) | 23 | (45.1%) | 22 | (45.8%) | 142 | (46.7%) | 0.126 |
| (n, %) | Female | 32 | (62.7%) | 28 | (52.8%) | 30 | (58.8%) | 18 | (36.0%) | 28 | (54.9%) | 26 | (54.2%) | 162 | (53.3%) | |
| | Total | 51 | | 53 | | 51 | | 50 | | 51 | | 48 | | 304 | | |
| Race/Ethnic | Caucasian | 31 | (60.8%) | 35 | (66.0%) | 34 | (66.7%) | 31 | (62.0%) | 37 | (72.5%) | 35 | (72.9%) | 203 | (66.8%) | 0.694 |
| Origin (n, %) | Black | 8 | (15.7%) | 8 | (15.1%) | 7 | (13.7%) | 7 | (14.0%) | 8 | (15.7%) | 5 | (10.4%) | 43 | (14.1%) | |
| [2] | Asian | 2 | (3.9%) | 2 | (3.8%) | 0 | (0.0%) | 0 | (0.0%) | 0 | (0.0%) | 2 | (4.2%) | 6 | (2.0%) | |
| | Hispanic | 9 | (17.6%) | 8 | (15.1%) | 9 | (17.6%) | 11 | (22.0%) | 5 | (9.8%) | 5 | (10.4%) | 47 | (15.5%) | |
| | Other | 1 | (2.0%) | 0 | (0.0%) | 1 | (2.0%) | 1 | (2.0%) | 1 | (2.0%) | 1 | (2.1%) | 5 | (1.6%) | |
| | Total | 51 | | 53 | | 51 | | 50 | | 51 | | 48 | | 304 | | |
| Height (cm) | N | 51 | | 53 | | 51 | | 50 | | 51 | | 48 | | 304 | | 0.888 |
| | Mean | 170.0 | | 171.7 | | 169.6 | | 170.2 | | 170.0 | | 170.9 | | 170.4 | | |
| | SD | 8.99 | | 9.91 | | 8.84 | | 9.90 | | 8.99 | | 9.11 | | 9.25 | | |
| | Median | 170.2 | | 170.2 | | 167.6 | | 170.2 | | 170.2 | | 170.6 | | 170.2 | | |
| | Range | 152.4-190.5 | | 152.0-195.6 | | 154.9-190.5 | | 149.9-198.1 | | 151.0-191.0 | | 157.5-190.5 | | 149.9-198.1 | | |
| Weight (kg) | N | 51 | | 53 | | 51 | | 50 | | 51 | | 48 | | 304 | | 0.528 |
| | Mean | 73.3 | | 75.3 | | 79.4 | | 73.4 | | 77.3 | | 76.7 | | 75.9 | | |
| | SD | 19.71 | | 14.32 | | 19.72 | | 21.59 | | 15.21 | | 19.94 | | 18.53 | | |
| | Median | 67.7 | | 74.5 | | 80.0 | | 66.5 | | 76.2 | | 72.5 | | 74.0 | | |
| | Range | 44.5-129.1 | | 45.4-112.7 | | 45.9-120.7 | | 44.9-147.7 | | 52.7-111.6 | | 48.6-157.8 | | 44.5-157.8 | | |
| Number of | 3 | 13 | (25.5%) | 18 | (34.0%) | 9 | (17.6%) | 10 | (20.0%) | 13 | (25.5%) | 16 | (33.3%) | 79 | (26.0%) | 0.297 |
| Third Molars | 4 | 36 | (70.6%) | 35 | (66.0%) | 39 | (76.5%) | 39 | (78.0%) | 38 | (74.5%) | 31 | (64.6%) | 218 | (71.7%) | |
| Extracted | 5 | 1 | (2.0%) | 0 | (0.0%) | 3 | (5.9%) | 1 | (2.0%) | 0 | (0.0%) | 0 | (0.0%) | 5 | (1.6%) | |
| (N, %) [3] | 6 | 1 | (2.0%) | 0 | (0.0%) | 0 | (0.0%) | 0 | (0.0%) | 0 | (0.0%) | 0 | (0.0%) | 1 | (0.3%) | |
| | 7 | 0 | (0.0%) | 0 | (0.0%) | 0 | (0.0%) | 0 | (0.0%) | 0 | (0.0%) | 1 | (2.1%) | 1 | (0.3%) | |
| | TOTAL | 51 | | 53 | | 51 | | 50 | | 51 | | 48 | | 304 | | |

| | | | | | | | | | |
|---|---|---|---|---|---|---|---|---|---|
| Time | N | 51 | 53 | 51 | 50 | 51 | 48 | 304 | 0.224 |
| Between End | Mean | 152.9 | 141.1 | 154.8 | 161.3 | 152.9 | 159.9 | 153.7 | |
| of Surgery | SD | 39.71 | 38.31 | 44.15 | 46.10 | 33.65 | 58.37 | 44.01 | |
| and Study | Median | 150.0 | 137.0 | 154.0 | 160.5 | 149.0 | 149.5 | 150.0 | |
| Medication | Range | 58.0-263.0 | 74.0-277.0 | 80.0-294.0 | 89.0-275.0 | 85.0-244.0 | 81.0-348.0 | 58.0-348.0 | |
| (Minutes) | | | | | | | | | |

[1] FOR AGE, HEIGH, WEIGHT, AND TIME BETWEEN END OF SURGERY AND STUDY MEDICATION, P-VALUES ARE FROM TWO-WAY ANALYSIS OF VARIANCE WITH TREATMENT AND SITE AS FACTORS; FOR GENDER, RACE/ETHNIC ORIGIN, AND NUMBER OF THIRD MOLARS EXTRACTED, P-VALUES ARE FROM COCHRAN-MANTEL-HAENZEL TEST ADJUSTING FOR SITE.
[2] BLACK, ASIAN, HISPANIC, AND OTHER ARE COMBINED INTO ONE CATEGORY TO DERIVE P-VALUE.
[3] 4 OR MORE THIRD MOLARS EXTRACTED AS ONE CATEGORY TO DERIVE P-VALUE.

TABLE 29A
Baseline Pain Intensity Scores
Intent-To-Treat Population, All Patients

| | | | P-VALUE FOR PAIRWISE COMPARISONS | | | | | P-Value |
|---|---|---|---|---|---|---|---|---|
| | | | | | MS 60 mg | MS 60 mg | MS 60 mg | for |
| PAIN INTENSITY | | | | NTX | NTX | NTX | NTX | Overall |
| TREATMENT | MODERATE | SEVERE | MS 60 mg | 0.01 mg | 0.001 mg | 0.01 mg | 0.1 mg | Treatment |
| Placebo | 25 (49.0%) | 26 (51.0%) | 0.989 | 0.994 | 0.935 | 1.000 | 0.916 | 0.949 |
| MS 60 mg | 26 (49.1%) | 27 (50.9%) | | 0.998 | 0.923 | 0.989 | 0.925 | |
| NTX 0.01 MG | 25 (49.0%) | 26 (51.0%) | | | 0.923 | 0.994 | 0.923 | |
| MS 60 mg/NTX 0.001 mg | 24 (48.0%) | 26 (52.0%) | | | | 0.935 | 0.851 | |
| MS 60 mg/NTX 0.01 mg | 25 (49.0%) | 26 (51.0%) | | | | | 0.916 | |
| MS 60 mg/NTX 0.1 mg | 24 (50.0%) | 24 (50.0%) | | | | | | |

NOTE:
P-VALUES ARE FROM COCHRAN-MANTEL-HAENZEL TEST ADJUSTING FOR SITE.

TABLE 29B
Baseline Visual Analog Scale (VAS) Scores
Intent-To-Treat Population, All Patients

| | | | | | | | | | | P-VALUE FOR PAIRWISE COMPARISONS | | | | | |
|---|---|---|---|---|---|---|---|---|---|---|---|---|---|---|---|
| | | | | | | | | | | | | MS | | MS | P-Value |
| BASELINE VAS SCORE | | | | | | | | | | | | 60 mg | MS 60 mg | 60 mg | for |
| | Moderate [1] | | | Severe [1] | | | Total | | | | NTX | NTX | NTX | NTX | Overall |
| TREATMENT | N | Mean | (SD) | N | Mean | (SD) | N | Mean | (SD) | MS 60 mg | 0.01 mg | 0.001 mg | 0.01 mg | 0.1 mg | Treatment |
| Placebo | 25 | 69.0 | (12.72) | 26 | 82.5 | (9.04) | 51 | 75.9 | (12.86) | 0.464 | 0.922 | 0.378 | 0.127 | 0.173 | 0.552 |
| MS 60 mg | 26 | 69.9 | (8.26) | 27 | 78.5 | (8.46) | 53 | 74.3 | (9.35) | | 0.527 | 0.871 | 0.418 | 0.511 | |
| NTX 0.01 mg | 25 | 69.8 | (10.08) | 26 | 81.3 | (7.29) | 51 | 75.7 | (10.45) | | | 0.433 | 0.153 | 0.205 | |
| MS 60 mg/ | 24 | 65.3 | (7.55) | 26 | 81.9 | (9.02) | 50 | 73.9 | (11.79) | | | | 0.524 | 0.624 | |
| NTX 0.001 mg | | | | | | | | | | | | | | | |
| MS 60 mg/ | 25 | 63.2 | (8.74) | 26 | 81.3 | (8.77) | 51 | 72.4 | (12.57) | | | | | 0.889 | |
| NTX 0.01 mg | | | | | | | | | | | | | | | |
| MS 60 mg/ | 24 | 64.8 | (7.85) | 24 | 80.7 | (7.64) | 48 | 72.8 | (11.09) | | | | | | |
| NTX 0.1 mg | | | | | | | | | | | | | | | |

NOTE:
CP-VALUES ARE FROM TWO-WAY ANALYSIS OF VARIANCE WITH TREATMENT AND SITE AS FACTORS.
[1] BASELINE PAIN INTENSITY ON THE CATEGORICAL SCALE.

The TOTPAR results (e.g., 4 hour, 6 hour, 8 hour) are summarized in Table 30. The 0.01 mg NTX only group and the placebo treatment group had the lowest mean TOTPAR scores. All 4 of the active treatment groups exhibited mean TOTPAR scores that were numerically higher than NTX alone or placebo. The combination treatments had a dose-response relation in the mean TOTPAR scores, i.e., the highest dose of NTX (0.1 mg) had the highest mean TOTPAR scores and the lowest dose of NTX (0.001 mg) had the lowest mean TOTPAR scores. This pattern (high-dose (0.1 mg NTX)>mid-dose (0.01 mg NTX)>low dose (0.001 mg NTX) was generally observed for pain relief variables throughout the study. The mean TOTPAR score for the 0.01 mg NTX combination treatment was higher than that for the MS alone treatment, whereas the 0.001 mg NTX combination treatment mean was comparable to or lower than that for the MS alone treatment.

TABLE 30
Total Pain Relief Scores
Intent-to-Treat Population, All Patients

| TOTAL PAIN RELIEF SCORE | | | | | | | | |
|---|---|---|---|---|---|---|---|---|
| TREATMENT | N | MEAN | SD | MIN | MEDIAN | MAX | SOURCE | P-VALUE [1] |

TOTAL PAIN RELIEF SCORE (0-4 HOURS)

| | | | | | | | | |
|---|---|---|---|---|---|---|---|---|
| A) Placebo | 51 | 1.55 | 2.469 | 0.0 | 0.00 | 11.3 | TREATMENT | <.001*** |
| B) MS 60 mg | 53 | 3.88 | 3.557 | 0.0 | 2.88 | 11.0 | SITE | 0.924 |
| C) NTX 0.01 mg | 51 | 1.40 | 2.461 | 0.0 | 0.00 | 10.4 | TREATMENT BY SITE | 0.518 |
| D) MS 60 mg/NTX 0.001 mg | 50 | 3.46 | 3.912 | 0.0 | 2.56 | 12.5 | A-B | 0.001** |
| E) MS 60 mg/NTX 0.01 mg | 51 | 4.22 | 4.023 | 0.0 | 3.88 | 14.5 | A-C | 0.786 |
| F) MS 60 mg/NTX 0.1 mg | 48 | 4.71 | 3.858 | 0.0 | 3.56 | 14.5 | A-D | 0.009** |
| | | | | | | | A-E | <.001*** |
| | | | | | | | A-F | <.001*** |
| | | | | | | | B-C | <.001*** |
| | | | | | | | B-D | 0.563 |
| | | | | | | | B-E | 0.601 |
| | | | | | | | B-F | 0.352 |
| | | | | | | | C-D | 0.004** |
| | | | | | | | C-E | <.001*** |
| | | | | | | | C-F | <.001*** |
| | | | | | | | D-E | 0.277 |
| | | | | | | | D-F | 0.140 |
| | | | | | | | E-F | 0.678 |

TOTAL PAIN RELIEF SCORE (0-6 HOURS)

| | | | | | | | | |
|---|---|---|---|---|---|---|---|---|
| A) Placebo | 51 | 2.78 | 4.608 | 0.0 | 0.00 | 19.3 | TREATMENT | <.001*** |
| B) MS 60 mg | 53 | 6.32 | 5.895 | 0.0 | 4.75 | 18.4 | SITE | 0.797 |
| C) NTX 0.01 mg | 51 | 2.14 | 3.897 | 0.0 | 0.00 | 16.4 | TREATMENT BY SITE | 0.370 |
| D) MS 60 mg/NTX 0.001 mg | 50 | 5.86 | 6.647 | 0.0 | 3.81 | 20.5 | A-B | 0.003** |
| E) MS 60 mg/NTX 0.01 mg | 51 | 6.92 | 6.468 | 0.0 | 5.88 | 22.5 | A-C | 0.560 |
| F) MS 60 mg/NTX 0.1 mg | 48 | 7.92 | 6.565 | 0.0 | 5.63 | 22.5 | A-D | 0.012* |
| | | | | | | | A-E | <.001*** |
| | | | | | | | A-F | <.001*** |
| | | | | | | | B-C | <.001*** |
| | | | | | | | B-D | 0.698 |
| | | | | | | | B-E | 0.585 |
| | | | | | | | B-F | 0.294 |
| | | | | | | | C-D | 0.002** |
| | | | | | | | C-E | <.001*** |
| | | | | | | | C-F | <.001*** |
| | | | | | | | D-E | 0.357 |
| | | | | | | | D-F | 0.159 |
| | | | | | | | E-F | 0.609 |

TOTAL PAIN RELIEF SCORE (0-8 HOURS)

| | | | | | | | | |
|---|---|---|---|---|---|---|---|---|
| A) Placebo | 51 | 4.00 | 6.759 | 0.0 | 0.00 | 26.3 | TREATMENT | <.001*** |
| B) MS 60 mg | 53 | 8.56 | 8.155 | 0.0 | 6.75 | 26.4 | SITE | 0.656 |
| C) NTX 0.01 mg | 51 | 2.86 | 5.339 | 0.0 | 0.00 | 22.4 | TREATMENT BY SITE | 0.312 |
| D) MS 60 mg/NTX 0.001 mg | 50 | 8.19 | 9.450 | 0.0 | 4.38 | 28.5 | A-B | 0.007** |
| E) MS 60 mg/NTX 0.01 mg | 51 | 9.58 | 9.049 | 0.0 | 7.88 | 30.5 | A-C | 0.485 |
| F) MS 60 mg/NTX 0.1 mg | 48 | 11.19 | 9.407 | 0.0 | 8.06 | 30.5 | A-D | 0.016* |
| | | | | | | | A-E | <.001*** |
| | | | | | | | A-F | <.001*** |
| | | | | | | | B-C | <.001*** |
| | | | | | | | B-D | 0.796 |
| | | | | | | | B-E | 0.514 |
| | | | | | | | B-F | 0.215 |
| | | | | | | | C-D | 0.002** |
| | | | | | | | C-E | <.001*** |
| | | | | | | | C-F | <.001*** |
| | | | | | | | D-E | 0.370 |
| | | | | | | | D-F | 0.142 |
| | | | | | | | E-F | 0.550 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, or <=0.001 RESPECTIVELY.

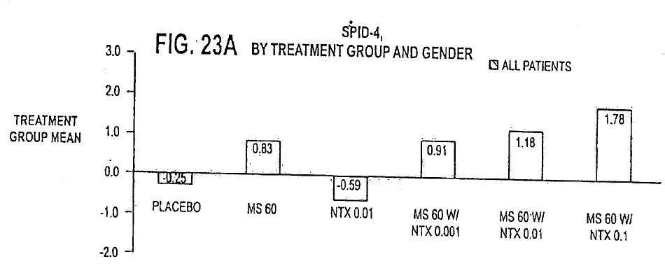

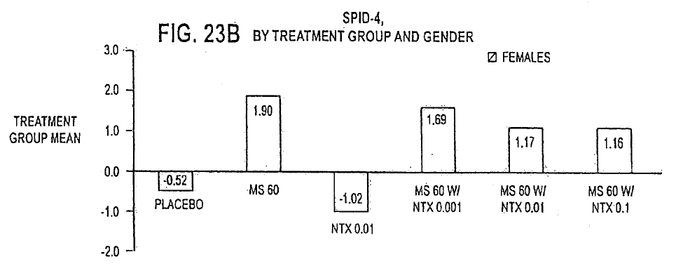

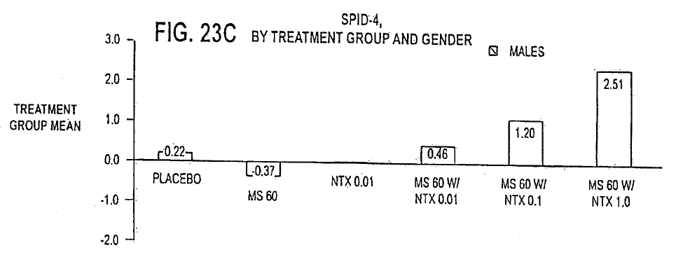

Table 31 summarizes the results of the 4, 6, and 8 hour SPID results. The 4 hour SPID results are also represented in FIG. 23A. The 0.01 mg NTX alone and placebo treatment groups had the lowest mean 4 hour SPID scores. All 4 of the active treatment groups with MS alone or in combination with NTX exhibited improved profiles in mean SPID relative to NTX alone or placebo. The mean 4 hour SPID scores for the 0.01 mg NTX and 0.1 mg NTX combination treatments were higher than that for the MS alone treatment, whereas the 0.001 mg NTX combination treatment was comparable to that for the MS alone treatment (FIG. 23A).

The patterns of the 6 hour and 8 hour SPID scores were similar to those at 4 hours.

TABLE 31
Sum of Pain Intensity Differences
Intent-To-Treat Population, All Patients

| SUM OF PAIN INTENSITY DIFFERENCES [1] | | | | | | | | |
|---|---|---|---|---|---|---|---|---|
| | N | MEAN | SD | MIN | MEDIAN | MAX | SOURCE | P-VALUE [2] |

SUM OF PAIN INTENSITY DIFFERENCES (0-4 HOURS)

| | | | | | | | | |
|---|---|---|---|---|---|---|---|---|
| A) Placebo | 51 | −0.25 | 2.293 | −4 | 0.00 | 6 | TREATMENT | 0.001** |
| B) MS 60 mg | 53 | 0.83 | 2.659 | −4 | 0.00 | 6 | SITE | 0.285 |
| C) NTX 0.01 mg | 51 | −0.59 | 2.370 | −4 | 0.00 | 7 | TREATMENT BY SITE | 0.559 |
| D) MS 60 mg/NTX 0.001 mg | 50 | 0.91 | 3.261 | −4 | 0.00 | 10 | A-B | 0.076 |
| E) MS 60 mg/NTX 0.01 mg | 51 | 1.18 | 3.157 | −4 | 0.00 | 11 | A-C | 0.522 |
| F) MS 60 mg/NTX 0.1 mg | 48 | 1.78 | 3.077 | −4 | 1.63 | 11 | A-D | 0.065 |
| | | | | | | | A-E | 0.021* |
| | | | | | | | A-F | 0.001** |
| | | | | | | | B-C | 0.016* |
| | | | | | | | B-D | 0.919 |
| | | | | | | | B-E | 0.585 |
| | | | | | | | B-F | 0.158 |
| | | | | | | | C-D | 0.013* |
| | | | | | | | C-E | 0.003** |
| | | | | | | | C-F | <.001*** |
| | | | | | | | D-E | 0.663 |
| | | | | | | | D-F | 0.197 |
| | | | | | | | E-F | 0.382 |

SUM OF PAIN INTENSITY DIFFERENCES (0-6 HOURS)

| | | | | | | | | |
|---|---|---|---|---|---|---|---|---|
| A) Placebo | 51 | −0.21 | 3.973 | −6 | 0.00 | 10 | TREATMENT | 0.001** |
| B) MS 60 mg | 53 | 1.44 | 4.385 | −6 | 0.00 | 11 | SITE | 0.153 |
| C) NTX 0.01 mg | 51 | −0.91 | 3.705 | −6 | 0.00 | 11 | TREATMENT BY SITE | 0.405 |
| D) MS 60 mg/NTX 0.001 mg | 50 | 1.77 | 5.375 | −6 | 0.00 | 16 | A-B | 0.096 |
| E) MS 60 mg/NTX 0.01 mg | 51 | 2.03 | 5.056 | −6 | 0.00 | 17 | A-C | 0.433 |
| F) MS 60 mg/NTX 0.1 mg | 48 | 3.06 | 5.146 | −6 | 1.81 | 17 | A-D | 0.050 |
| | | | | | | | A-E | 0.026* |
| | | | | | | | A-F | 0.002** |
| | | | | | | | B-C | 0.014* |
| | | | | | | | B-D | 0.742 |
| | | | | | | | B-E | 0.567 |
| | | | | | | | B-F | 0.157 |
| | | | | | | | C-D | 0.006** |
| | | | | | | | C-E | 0.002** |
| | | | | | | | C-F | <.001*** |
| | | | | | | | D-E | 0.814 |
| | | | | | | | D-F | 0.285 |
| | | | | | | | E-F | 0.397 |

SUM OF PAIN INTENSITY DIFFERENCES (0-8 HOURS)

| | | | | | | | | |
|---|---|---|---|---|---|---|---|---|
| A) Placebo | 51 | −0.20 | 5.641 | −8 | 0.00 | 13 | TREATMENT | 0.001** |
| B) MS 60 mg | 53 | 1.87 | 6.021 | −8 | 0.00 | 15 | SITE | 0.092 |
| C) NTX 0.01 mg | 51 | −1.23 | 5.046 | −8 | 0.00 | 15 | TREATMENT BY SITE | 0.368 |
| D) MS 60 mg/NTX 0.001 mg | 50 | 2.50 | 7.411 | −8 | 0.00 | 22 | A-B | 0.132 |
| E) MS 60 mg/NTX 0.01 mg | 51 | 2.92 | 7.111 | −8 | 0.00 | 23 | A-C | 0.421 |
| F) MS 60 mg/NTX 0.1 mg | 48 | 4.32 | 7.247 | −8 | 3.00 | 23 | A-D | 0.054 |
| | | | | | | | A-E | 0.025* |
| | | | | | | | A-F | 0.002** |
| | | | | | | | B-C | 0.021* |
| | | | | | | | B-D | 0.654 |
| | | | | | | | B-E | 0.455 |
| | | | | | | | B-F | 0.123 |
| | | | | | | | C-D | 0.007** |
| | | | | | | | C-E | 0.002** |
| | | | | | | | C-F | <.001*** |
| | | | | | | | D-E | 0.773 |
| | | | | | | | D-F | 0.281 |
| | | | | | | | E-F | 0.421 |

[1] PAIN INTENSITY DIFFERENCE = PAIN INTENSITY AT BASELINE − PAIN INTENSITY AT CURRENT TIME.
[2] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, or <=0.001 RESPECTIVELY.

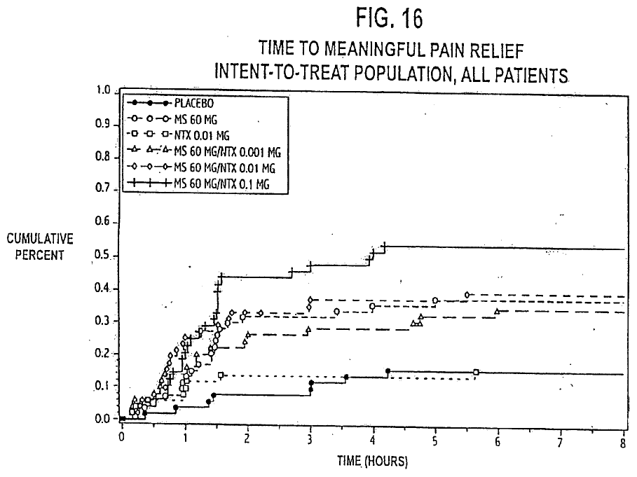

FIG. 16 is a visual presentation of the summary and analysis of time to onset of meaningful pain relief presented in Table 32A. The median time to onset of meaningful pain relief was shortest in the 0.1 mg NTX combination treatment group.

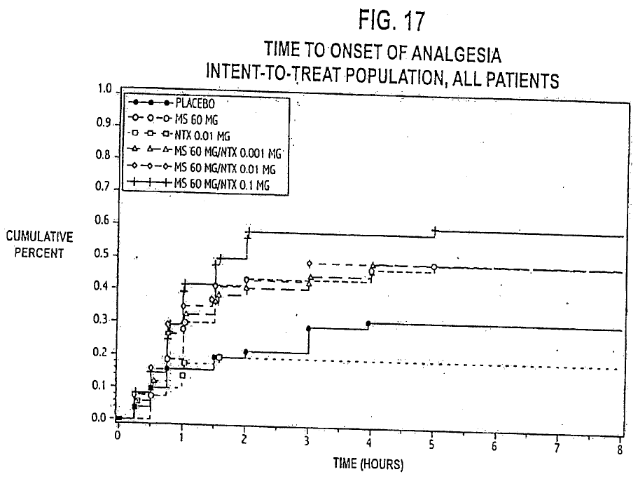

FIG. 17 is a visual presentation of the summary and analysis of time to onset of analgesia presented in Table 32B. The median time to onset of analgesia was shortest in the 0.1 mg NTX combination treatment group.

TABLE 32A
Time To Onset of Meaningful Pain Relief
Intent-To-Treat Population, All Patients

| | | | 95% | | | |
|---|---|---|---|---|---|---|
| | | MEDIAN | CONFIDENCE | | | |
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 51 | >8:00 | (>8:00, >8:00) | TREATMENT | <.001*** | <.001*** |
| B) MS 60 mg | 53 | >8:00 | (5:00, >8:00) | A-B | 0.024* | 0.016* |
| C) NTX 0.01 mg | 51 | >8:00 | (>8:00, >8:00) | A-C | 0.965 | 0.899 |
| D) MS 60 mg/NTX 0.001 mg | 50 | >8:00 | (>8:00, >8:00) | A-D | 0.054 | 0.031* |
| E) MS 60 mg/NTX 0.01 mg | 51 | >8:00 | (3:00, >8:00) | A-E | 0.008** | 0.004** |
| F) MS 60 mg/NTX 0.1 mg | 48 | 3:58 | (1:31, >8:00) | A-F | <.001*** | <.001*** |
| | | | | B-C | 0.028* | 0.025* |
| | | | | B-D | 0.783 | 0.859 |
| | | | | B-E | 0.664 | 0.574 |
| | | | | B-F | 0.046* | 0.094 |
| | | | | C-D | 0.062 | 0.046* |
| | | | | C-E | 0.010* | 0.006** |
| | | | | C-F | <.001*** | <.001*** |
| | | | | D-E | 0.488 | 0.474 |
| | | | | D-F | 0.026* | 0.073 |
| | | | | E-F | 0.127 | 0.286 |

*, **, ***P-VALUE <=0.05, <=0.001, OR <=0.001 RESPECTIVELY.

TABLE 32B
Time to Onset of Analgesia
Intent-To-Treat Population, All Patients

| | | | 95% | | | |
|---|---|---|---|---|---|---|
| | | MEDIAN | CONFIDENCE | | | |
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 51 | >8:00 | (>8:00, >8:00) | TREATMENT | 0.001** | <.001*** |
| B) MS 60 mg | 53 | >8:00 | (1:30, >8:00) | A-B | 0.099 | 0.094 |
| C) NTX 0.01 mg | 51 | >8:00 | (>8:00, >8:00) | A-C | 0.373 | 0.325 |
| D) MS 60 mg/NTX 0.001 mg | 50 | >8:00 | (1:30, >8:00) | A-D | 0.077 | 0.060 |
| E) MS 60 mg/NTX 0.01 mg | 51 | >8:00 | (1:27, >8:00) | A-E | 0.054 | 0.027* |
| F) MS 60 mg/NTX 0.1 mg | 48 | 1:47 | (1:00, >8:00) | A-F | 0.002** | 0.003** |
| | | | | B-C | 0.011* | 0.008** |
| | | | | B-D | 0.866 | 0.787 |
| | | | | B-E | 0.744 | 0.541 |
| | | | | B-F | 0.143 | 0.179 |
| | | | | C-D | 0.008** | 0.004** |
| | | | | C-E | 0.005** | 0.001** |
| | | | | C-F | <.001*** | <.001*** |
| | | | | D-E | 0.878 | 0.740 |
| | | | | D-F | 0.207 | 0.302 |
| | | | | E-F | 0.265 | 0.486 |

*, **, ***P-VALUE <=0.05, <=0.001, OR <=0.001 RESPECTIVELY.

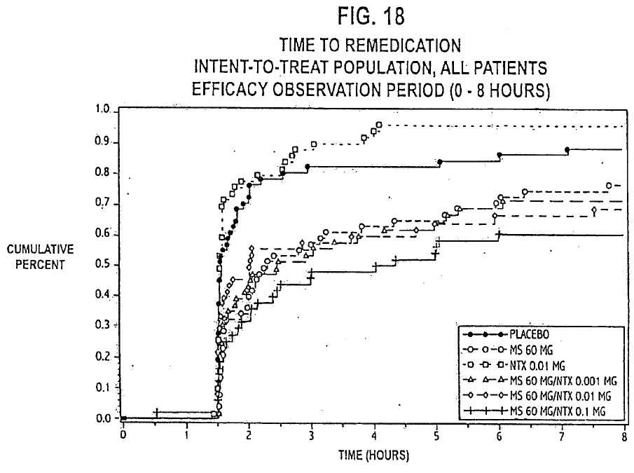

FIGS. 18 and 19 are a visual presentation of the summary and analysis of time to remedication (rescue medication) up to 8 and 24 hours presented in Table 33. The survival distributions (0-8 hours) were different across treatment groups. The cumulative percent distributions were different for the MS alone or in combination with NTX compared to 0.01 mg NTX alone or placebo (FIG. 18). The median times to administration of rescue medication were longer for the MS alone or in combination with NTX treatment groups compared to the 0.01 mg NTX alone and placebo groups. The longest duration of action was observed in the 0.1 mg NTX combination treatment group, followed by the 0.001 mg NTX combination treatment group.

The cumulative percent distributions (0-24 hours) were also different across treatment groups, and were also different for the MS alone or in combination with NTX groups compared to the 0.01 mg NTX alone or placebo group (FIG. 19). Again, the median times to administration of rescue medication were longer for the morphine and combination treatment groups.

Analyses of time to remedication up to 24 hours yielded generally similar results, however, the data should be viewed with caution because subjects were not under close supervision after 8 hours.

TABLE 33
Time To Rescue Medication
Intent-To-Treat Population, All Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |

EFFICACY OBSERVATION PERIOD (0-8 HOURS)

| | | | | | | |
|---|---|---|---|---|---|---|
| A) Placebo | 51 | 1:34 | (1:32, 1:48) | TREATMENT | <.001*** | <.001*** |
| B) MS 60 mg | 53 | 2:19 | (2:01, 4:21) | A-B | 0.001** | <.001*** |
| C) NTX 0.01 mg | 51 | 1:34 | (1:32, 1:36) | A-C | 0.140 | 0.872 |
| D) MS 60 mg/NTX 0.001 mg | 50 | 2:29 | (1:47, 5:01) | A-D | 0.001** | <.001*** |
| E) MS 60 mg/NTX 0.01 mg | 51 | 2:03 | (1:35, 5:00) | A-E | 0.002** | 0.003** |
| F) MS 60 mg/NTX 0.1 mg | 48 | 4:12 | (2:09, >8:00) | A-F | <.001*** | <.001*** |
| | | | | B-C | <.001*** | <.001*** |
| | | | | B-D | 0.871 | 0.907 |
| | | | | B-E | 0.960 | 0.412 |
| | | | | B-F | 0.309 | 0.303 |
| | | | | C-D | <.001*** | <.001*** |
| | | | | C-E | <.001*** | 0.001** |
| | | | | C-F | <.001*** | <.001*** |
| | | | | D-E | 0.838 | 0.495 |
| | | | | D-F | 0.407 | 0.270 |
| | | | | E-F | 0.305 | 0.079 |

EFFICACY OBSERVATION PERIOD (0-24 HOURS)

| | | | | | | |
|---|---|---|---|---|---|---|
| A) Placebo | 51 | 1:34 | (1:32, 1:48) | TREATMENT | <.001*** | <.001*** |
| B) MS 60 mg | 53 | 2:19 | (2:01, 4:21) | A-B | 0.002** | <.001*** |
| C) NTX 0.01 mg | 51 | 1:34 | (1:32, 1:36) | A-C | 0.056 | 0.866 |
| D) MS 60 mg/NTX 0.001 mg | 50 | 2:29 | (1:47, 5:01) | A-D | <.001*** | <.001*** |
| E) MS 60 mg/NTX 0.01 mg | 51 | 2:03 | (1:35, 5:00) | A-E | 0.002** | 0.002** |
| F) MS 60 mg/NTX 0.1 mg | 48 | 4:12 | (2:09, 8:48) | A-F | <.001*** | <.001*** |
| | | | | B-C | <.001*** | <.001*** |
| | | | | B-D | 0.660 | 0.973 |
| | | | | B-E | 0.913 | 0.459 |
| | | | | B-F | 0.154 | 0.219 |
| | | | | C-D | <.001*** | <.001*** |
| | | | | C-E | <.001*** | 0.001** |
| | | | | C-F | <.001*** | <.001*** |
| | | | | D-E | 0.748 | 0.458 |
| | | | | D-F | 0.332 | 0.251 |
| | | | | E-F | 0.199 | 0.062 |

*, **, ***P-VALUE <=0.05, <=0.01, or <=0.001 RESPECTIVELY.

Table 34 presents the summary and analysis of percent of subjects who took rescue medication up to 8 and 24 hours. Approximately 40% of subjects in the high-dose NTX (0.1 mg) combination group and more than 30% of subjects in the mid-dose NTX (0.01 mg) and low-dose NTX (0.001 mg) combination groups did not require rescue medication during 8 hours. Thus, the longest duration of action was observed in the 0.1 mg NTX combination treatment group. Analyses of the percentage of subjects who remedicated within 24 hours indicated that the NTX (0.001 mg, 0.01 mg, 0.1 mg) combination treatment groups were comparable and different from the placebo, 0.01 mg NTX and MS alone treatment groups, however, the data should be interpreted with caution because subjects were not under close supervision after 8 hours.

TABLE 34
Percent of Patients Rescued
Intent-To-Treat Population, All Patients

| | RESCUED | | | |
|---|---|---|---|---|
| TREATMENT | YES | NO | SOURCE | P-VALUE [1] |

EFFICACY OBSERVATION PERIOD (0-8 HOURS)

| | | | | |
|---|---|---|---|---|
| A) Placebo | 45 (88.2%) | 6 (11.8%) | TREATMENT | <.001*** |
| B) MS 60 mg | 40 (75.5%) | 13 (24.5%) | A-B | 0.092 |
| C) NTX 0.01 mg | 48 (94.1%) | 3 (5.9%) | A-C | 0.302 |
| D) MS 60 mg/NTX 0.001 mg | 34 (68.0%) | 16 (32.0%) | A-D | 0.015* |
| E) MS 60 mg/NTX 0.01 mg | 34 (66.7%) | 17 (33.3%) | A-E | 0.008** |
| F) MS 60 mg/NTX 0.1 mg | 29 (60.4%) | 19 (39.6%) | A-F | 0.001** |
| | | | B-C | 0.008** |
| | | | B-D | 0.400 |
| | | | B-E | 0.322 |
| | | | B-F | 0.103 |
| | | | C-D | <.001*** |
| | | | C-E | <.001*** |
| | | | C-F | <.001*** |
| | | | D-E | 0.840 |
| | | | D-F | 0.391 |
| | | | E-F | 0.532 |

EFFICACY OBSERVATION PERIOD (0-24 HOURS)

| | | | | |
|---|---|---|---|---|
| A) Placebo | 49 (96.1%) | 2 (3.9%) | TREATMENT | 0.005** |
| B) MS 60 mg | 49 (92.5%) | 4 (7.5%) | A-B | 0.427 |
| C) NTX 0.01 mg | 50 (98.0%) | 1 (2.0%) | A-C | 0.558 |
| D) MS 60 mg/NTX 0.001 mg | 42 (84.0%) | 8 (16.0%) | A-D | 0.045* |
| E) MS 60 mg/NTX 0.01 mg | 43 (84.3%) | 8 (15.7%) | A-E | 0.042* |
| F) MS 60 mg/NTX 0.1 mg | 37 (77.1%) | 11 (22.9%) | A-F | 0.004** |
| | | | B-C | 0.182 |
| | | | B-D | 0.179 |
| | | | B-E | 0.194 |
| | | | B-F | 0.030* |
| | | | C-D | 0.013* |
| | | | C-E | 0.013* |
| | | | C-F | 0.001** |
| | | | D-E | 0.999 |
| | | | D-F | 0.367 |
| | | | E-F | 0.369 |

P-VALUES ARE FROM COCHRAN-MANTEL-HAENZEL TEST ADJUSTING FOR SITE.

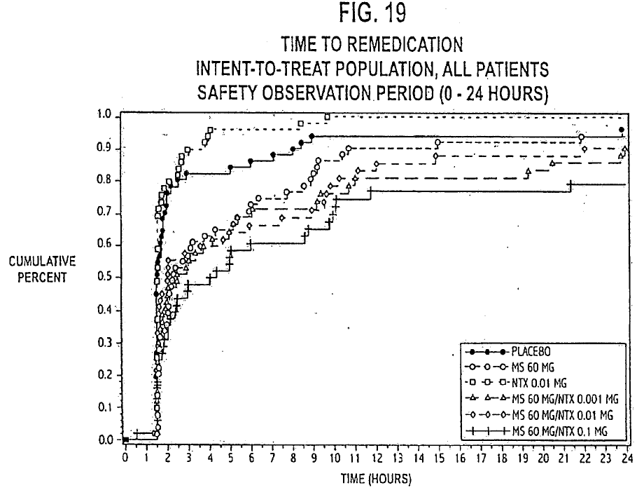

FIG. 20 is a visual presentation of the hourly pain relief scores presented in Table 35. The hourly pain relief scores for the 0.01 mg NTX alone or placebo treatment were less than those for the active treatment groups (MS alone or in combination with NTX) which improved over time. There was separation between the 0.01 mg NTX alone or placebo and the active treatment groups that continued throughout the 8 hour study period. Highest pain relief scores were observed for the 0.1 mg NTX combination group followed by the 0.01 mg NTX combination group (FIG. 20).

TABLE 35
Pain Relief (PR) Scores
Intent-To-Treat Population, All Patients

| | PAIN RELIEF | | | | | | |
|---|---|---|---|---|---|---|---|
| | SCORE (PR) | | | | | | |
| TREATMENT | N | MEAN | SD | MIN | MAX | SOURCE | P-VALUE [1] |
| 15 MINUTES | | | | | | | |
| A) Placebo | 51 | 0.12 | 0.382 | 0 | 2 | Treatment | 0.716 |
| B) MS 60 mg | 53 | 0.11 | 0.375 | 0 | 2 | Site | 0.031* |
| C) NTX 0.01 mg | 51 | 0.20 | 0.530 | 0 | 2 | Treatment by Site | 0.886 |
| D) MS 60 mg/NTX 0.001 mg | 50 | 0.24 | 0.517 | 0 | 2 | A-B | N/D |
| E) MS 60 mg/NTX 0.01 mg | 51 | 0.24 | 0.619 | 0 | 3 | A-C | N/D |
| F) MS 60 mg/NTX 0.1 mg | 48 | 0.19 | 0.532 | 0 | 2 | A-D | N/D |
| | | | | | | A-E | N/D |
| | | | | | | A-F | N/D |
| | | | | | | B-C | N/D |
| | | | | | | B-D | N/D |
| | | | | | | B-E | N/D |
| | | | | | | B-F | N/D |
| | | | | | | C-D | N/D |
| | | | | | | C-E | N/D |
| | | | | | | C-F | N/D |
| | | | | | | D-E | N/D |
| | | | | | | D-F | N/D |
| | | | | | | E-F | N/D |
| 30 MINUTES | | | | | | | |
| A) Placebo | 51 | 0.29 | 0.540 | 0 | 2 | Treatment | 0.459 |
| B) MS 60 mg | 53 | 0.32 | 0.581 | 0 | 2 | Site | 0.107 |
| C) NTX 0.01 mg | 51 | 0.29 | 0.610 | 0 | 3 | Treatment by Site | 0.378 |
| D) MS 60 mg/NTX 0.001 mg | 50 | 0.26 | 0.487 | 0 | 2 | A-B | N/D |
| E) MS 60 mg/NTX 0.01 mg | 51 | 0.47 | 0.857 | 0 | 4 | A-C | N/D |
| F) MS 60 mg/NTX 0.1 mg | 48 | 0.44 | 0.741 | 0 | 3 | A-D | N/D |
| | | | | | | A-E | N/D |
| | | | | | | A-F | N/D |
| | | | | | | B-C | N/D |
| | | | | | | B-D | N/D |
| | | | | | | B-E | N/D |
| | | | | | | B-F | N/D |
| | | | | | | C-D | N/D |
| | | | | | | C-E | N/D |
| | | | | | | C-F | N/D |
| | | | | | | D-E | N/D |
| | | | | | | D-F | N/D |
| | | | | | | E-F | N/D |
| 45 MINUTES | | | | | | | |
| A) Placebo | 51 | 0.29 | 0.540 | 0 | 2 | Treatment | 0.017* |
| B) MS 60 mg | 53 | 0.64 | 0.762 | 0 | 3 | Site | 0.464 |
| C) NTX 0.01 mg | 51 | 0.35 | 0.658 | 0 | 3 | Treatment by Site | 0.481 |
| D) MS 60 mg/NTX 0.001 mg | 50 | 0.58 | 0.835 | 0 | 3 | A-B | 0.054 |
| E) MS 60 mg/NTX 0.01 mg | 51 | 0.84 | 1.065 | 0 | 4 | A-C | 0.875 |
| F) MS 60 mg/NTX 0.1 mg | 48 | 0.65 | 0.863 | 0 | 4 | A-D | 0.137 |
| | | | | | | A-E | 0.001** |
| | | | | | | A-F | 0.080 |
| | | | | | | B-C | 0.079 |
| | | | | | | B-D | 0.685 |
| | | | | | | B-E | 0.216 |
| | | | | | | B-F | 0.900 |
| | | | | | | C-D | 0.185 |
| | | | | | | C-E | 0.003** |
| | | | | | | C-F | 0.111 |
| | | | | | | D-E | 0.106 |
| | | | | | | D-F | 0.785 |
| | | | | | | E-F | 0.183 |
| 1 HOUR | | | | | | | |
| A) Placebo | 51 | 0.31 | 0.616 | 0 | 3 | Treatment | 0.002** |
| B) MS 60 mg | 53 | 0.87 | 0.962 | 0 | 4 | Site | 0.478 |
| C) NTX 0.01 mg | 51 | 0.47 | 0.809 | 0 | 3 | Treatment by Site | 0.687 |
| D) MS 60 mg/NTX 0.001 mg | 50 | 0.76 | 1.041 | 0 | 4 | A-B | 0.004** |
| E) MS 60 mg/NTX 0.01 mg | 51 | 0.96 | 1.038 | 0 | 4 | A-C | 0.510 |
| F) MS 60 mg/NTX 0.1 mg | 48 | 0.96 | 1.010 | 0 | 4 | A-D | 0.033* |
| | | | | | | A-E | 0.001** |
| | | | | | | A-F | 0.002** |
| | | | | | | B-C | 0.029* |
| | | | | | | B-D | 0.499 |
| | | | | | | B-E | 0.650 |
| | | | | | | B-F | 0.767 |
| | | | | | | C-D | 0.141 |
| | | | | | | C-E | 0.009** |
| | | | | | | C-F | 0.016* |
| | | | | | | D-E | 0.264 |
| | | | | | | D-F | 0.343 |
| | | | | | | E-F | 0.881 |
| 1.5 HOURS | | | | | | | |
| A) Placebo | 51 | 0.35 | 0.658 | 0 | 3 | Treatment | <.001*** |
| B) MS 60 mg | 53 | 1.13 | 1.038 | 0 | 3 | Site | 0.863 |
| C) NTX 0.01 mg | 51 | 0.39 | 0.723 | 0 | 3 | Treatment by Site | 0.479 |
| D) MS 60 mg/NTX 0.001 mg | 50 | 0.98 | 1.169 | 0 | 4 | A-B | <.001*** |
| E) MS 60 mg/NTX 0.01 mg | 51 | 1.22 | 1.154 | 0 | 4 | A-C | 0.905 |
| F) MS 60 mg/NTX 0.1 mg | 48 | 1.31 | 1.095 | 0 | 4 | A-D | 0.002** |
| | | | | | | A-E | <.001*** |
| | | | | | | A-F | <.001*** |
| | | | | | | B-C | <.001*** |
| | | | | | | B-D | 0.462 |
| | | | | | | B-E | 0.699 |
| | | | | | | B-F | 0.565 |
| | | | | | | C-D | 0.003** |
| | | | | | | C-E | <.001*** |
| | | | | | | C-F | <.001*** |
| | | | | | | D-E | 0.267 |
| | | | | | | D-F | 0.200 |
| | | | | | | E-F | 0.846 |
| 2 HOURS | | | | | | | |
| A) Placebo | 51 | 0.35 | 0.658 | 0 | 3 | Treatment | <.001*** |
| B) MS 60 mg | 53 | 1.21 | 1.150 | 0 | 3 | Site | 0.926 |
| C) NTX 0.01 mg | 51 | 0.37 | 0.692 | 0 | 3 | Treatment by Site | 0.519 |
| D) MS 60 mg/NTX 0.001 mg | 50 | 1.02 | 1.237 | 0 | 4 | A-B | <.001*** |
| E) MS 60 mg/NTX 0.01 mg | 51 | 1.16 | 1.173 | 0 | 4 | A-C | 0.944 |
| F) MS 60 mg/NTX 0.1 mg | 48 | 1.40 | 1.250 | 0 | 4 | A-D | 0.002** |
| | | | | | | A-E | <.001*** |
| | | | | | | A-F | <.001*** |
| | | | | | | B-C | <.001*** |
| | | | | | | B-D | 0.405 |
| | | | | | | B-E | 0.866 |
| | | | | | | B-F | 0.540 |
| | | | | | | C-D | 0.003** |
| | | | | | | C-E | <.001*** |
| | | | | | | C-F | <.001*** |
| | | | | | | D-E | 0.508 |
| | | | | | | D-F | 0.158 |
| | | | | | | E-F | 0.440 |
| 3 HOURS | | | | | | | |
| A) Placebo | 51 | 0.51 | 0.925 | 0 | 4 | Treatment | <.001*** |
| B) MS 60 mg | 53 | 1.18 | 1.180 | 0 | 3 | Site | 0.830 |
| C) NTX 0.01 mg | 51 | 0.35 | 0.716 | 0 | 3 | Treatment by Site | 0.641 |
| D) MS 60 mg/NTX 0.001 mg | 50 | 1.06 | 1.331 | 0 | 4 | A-B | 0.005** |
| E) MS 60 mg/NTX 0.01 mg | 51 | 1.31 | 1.288 | 0 | 4 | A-C | 0.503 |
| F) MS 60 mg/NTX 0.1 mg | 48 | 1.50 | 1.321 | 0 | 4 | A-D | 0.021* |
| | | | | | | A-E | <.001*** |
| | | | | | | A-F | <.001*** |
| | | | | | | B-C | <.001*** |
| | | | | | | B-D | 0.657 |
| | | | | | | B-E | 0.531 |
| | | | | | | B-F | 0.253 |
| | | | | | | C-D | 0.003** |
| | | | | | | C-E | <.001*** |
| | | | | | | C-F | <.001*** |
| | | | | | | D-E | 0.291 |
| | | | | | | D-F | 0.120 |
| | | | | | | E-F | 0.599 |
| 4 HOURS | | | | | | | |
| A) Placebo | 51 | 0.61 | 1.078 | 0 | 4 | Treatment | <.001*** |
| B) MS 60 mg | 53 | 1.26 | 1.273 | 0 | 3 | Site | 0.558 |
| C) NTX 0.01 mg | 51 | 0.37 | 0.747 | 0 | 3 | Treatment by Site | 0.460 |
| D) MS 60 mg/NTX 0.001 mg | 50 | 1.18 | 1.410 | 0 | 4 | A-B | 0.010* |
| E) MS 60 mg/NTX 0.01 mg | 51 | 1.37 | 1.326 | 0 | 4 | A-C | 0.360 |
| F) MS 60 mg/NTX 0.1 mg | 48 | 1.67 | 1.449 | 0 | 4 | A-D | 0.029* |
| | | | | | | A-E | 0.003** |
| | | | | | | A-F | <.001*** |
| | | | | | | B-C | <.001*** |
| | | | | | | B-D | 0.737 |
| | | | | | | B-E | 0.665 |
| | | | | | | B-F | 0.193 |
| | | | | | | C-D | 0.002** |
| | | | | | | C-E | <.001*** |
| | | | | | | C-F | <.001*** |
| | | | | | | D-E | 0.448 |
| | | | | | | D-F | 0.109 |
| | | | | | | E-F | 0.383 |
| 5 HOURS | | | | | | | |
| A) Placebo | 51 | 0.61 | 1.097 | 0 | 4 | Treatment | <.001*** |
| B) MS 60 mg | 53 | 1.25 | 1.285 | 0 | 4 | Site | 0.467 |
| C) NTX 0.01 mg | 51 | 0.37 | 0.747 | 0 | 3 | Treatment by Site | 0.161 |
| D) MS 60 mg/NTX 0.001 mg | 50 | 1.22 | 1.461 | 0 | 4 | A-B | 0.014* |
| E) MS 60 mg/NTX 0.01 mg | 51 | 1.37 | 1.341 | 0 | 4 | A-C | 0.364 |
| F) MS 60 mg/NTX 0.1 mg | 48 | 1.56 | 1.443 | 0 | 4 | A-D | 0.019* |
| | | | | | | A-E | 0.002** |
| | | | | | | A-F | 0.001** |
| | | | | | | B-C | <.001*** |
| | | | | | | B-D | 0.944 |
| | | | | | | B-E | 0.560 |
| | | | | | | B-F | 0.385 |
| | | | | | | C-D | 0.001** |
| | | | | | | C-E | <.001*** |
| | | | | | | C-F | <.001*** |
| | | | | | | D-E | 0.521 |
| | | | | | | D-F | 0.357 |
| | | | | | | E-F | 0.767 |
| 6 HOURS | | | | | | | |
| A) Placebo | 51 | 0.65 | 1.180 | 0 | 4 | Treatment | <.001*** |
| B) MS 60 mg | 53 | 1.13 | 1.194 | 0 | 4 | Site | 0.385 |
| C) NTX 0.01 mg | 51 | 0.35 | 0.716 | 0 | 3 | Treatment by Site | 0.236 |
| D) MS 60 mg/NTX 0.001 mg | 50 | 1.18 | 1.466 | 0 | 4 | A-B | 0.060 |
| E) MS 60 mg/NTX 0.01 mg | 51 | 1.27 | 1.313 | 0 | 4 | A-C | 0.243 |
| F) MS 60 mg/NTX 0.1 mg | 48 | 1.63 | 1.482 | 0 | 4 | A-D | 0.053 |
| | | | | | | A-E | 0.015* |
| | | | | | | A-F | <.001*** |
| | | | | | | B-C | 0.002** |
| | | | | | | B-D | 0.932 |
| | | | | | | B-E | 0.567 |
| | | | | | | B-F | 0.122 |
| | | | | | | C-D | 0.002** |
| | | | | | | C-E | <.001*** |
| | | | | | | C-F | <.001*** |
| | | | | | | D-E | 0.633 |
| | | | | | | D-F | 0.151 |
| | | | | | | E-F | 0.327 |
| 7 HOURS | | | | | | | |
| A) Placebo | 51 | 0.59 | 1.080 | 0 | 4 | Treatment | <.001*** |
| B) MS 60 mg | 53 | 1.11 | 1.204 | 0 | 4 | Site | 0.362 |
| C) NTX 0.01 mg | 51 | 0.37 | 0.747 | 0 | 3 | Treatment by Site | 0.194 |
| D) MS 60 mg/NTX 0.001 mg | 50 | 1.16 | 1.448 | 0 | 4 | A-B | 0.035* |
| E) MS 60 mg/NTX 0.01 mg | 51 | 1.35 | 1.397 | 0 | 4 | A-C | 0.433 |
| F) MS 60 mg/NTX 0.1 mg | 48 | 1.65 | 1.495 | 0 | 4 | A-D | 0.035* |
| | | | | | | A-E | 0.002** |
| | | | | | | A-F | <.001*** |
| | | | | | | B-C | 0.004** |
| | | | | | | B-D | 0.966 |
| | | | | | | B-E | 0.324 |
| | | | | | | B-F | 0.095 |
| | | | | | | C-D | 0.004** |
| | | | | | | C-E | <.001*** |
| | | | | | | C-F | <.001*** |
| | | | | | | D-E | 0.355 |
| | | | | | | D-F | 0.110 |
| | | | | | | E-F | 0.483 |
| 8 HOURS | | | | | | | |
| A) Placebo | 51 | 0.61 | 1.115 | 0 | 4 | Treatment | <.001*** |
| B) MS 60 mg | 53 | 1.11 | 1.204 | 0 | 4 | Site | 0.458 |
| C) NTX 0.01 mg | 51 | 0.35 | 0.716 | 0 | 3 | Treatment by Site | 0.202 |
| D) MS 60 mg/NTX 0.001 mg | 50 | 1.16 | 1.476 | 0 | 4 | A-B | 0.049* |
| E) MS 60 mg/NTX 0.01 mg | 51 | 1.33 | 1.409 | 0 | 4 | A-C | 0.317 |
| F) MS 60 mg/NTX 0.1 mg | 48 | 1.63 | 1.468 | 0 | 4 | A-D | 0.048* |
| | | | | | | A-E | 0.004** |
| | | | | | | A-F | <.001*** |
| | | | | | | B-C | 0.003** |
| | | | | | | B-D | 0.966 |
| | | | | | | B-E | 0.360 |
| | | | | | | B-F | 0.110 |
| | | | | | | C-D | 0.003** |
| | | | | | | C-E | <.001*** |
| | | | | | | C-F | <.001*** |
| | | | | | | D-E | 0.392 |
| | | | | | | D-F | 0.127 |
| | | | | | | E-F | 0.487 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, or <=0.001 RESPECTIVELY.
N/D: NOTE DONE (BECAUSE OVERALL P-VALUE NOT SIGNIFICANT).

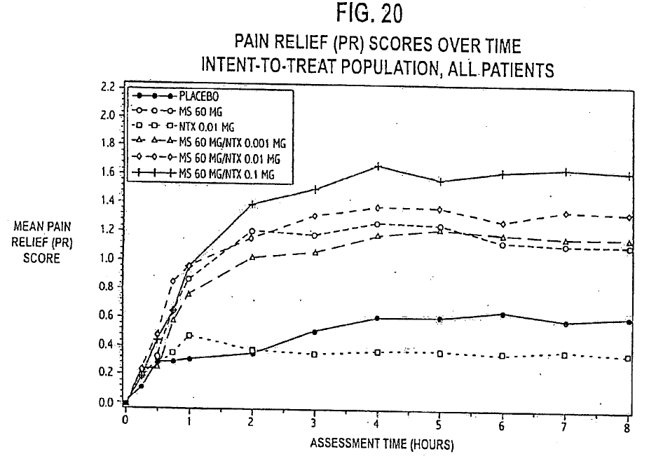

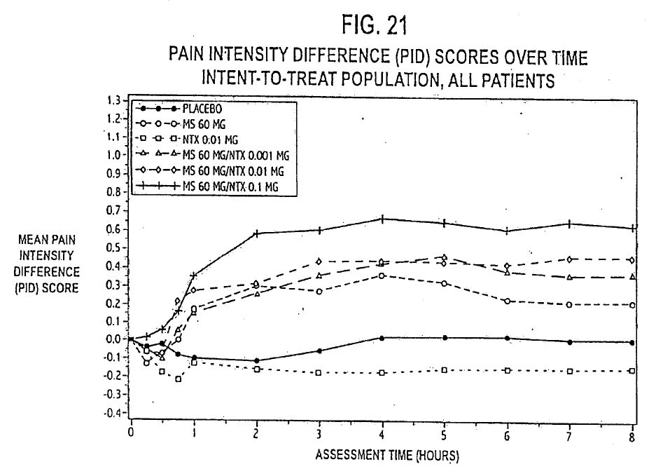

The hourly pain intensity difference (PID) scores are presented in Table 36 and FIG. 21. The hourly PID scores for the 0.01 mg NTX alone and placebo treatment groups were generally flat while the hourly PID scores generally improved over time for the active treatment groups (MS alone or in combination with NTX). The mean scores for the morphine and morphine/naltrexone groups were higher than the mean PD scores for the 0.01 mg NTX alone or placebo group at each assessment time from 1-8 hours. Highest pain relief as measured by mean PID scores was observed for the high-dose (0.1 mg NTX) combination group.

TABLE 36
Pain Intensity Difference (PID) Scores
Intent-To-Treat Population, All Patients

| | Pain Intensity | | | | | | |
|---|---|---|---|---|---|---|---|
| | Difference | | | | | | |
| | Score (PID) | | | | | | |
| Treatment | N | Mean | SD | Min | Max | Source | P-Value [1] |
| 15 MINUTES | | | | | | | |
| A) Placebo | 51 | −0.04 | 0.344 | −1 | 1 | Treatment | 0.650 |
| B) MS 60 mg | 53 | −0.13 | 0.342 | −1 | 0 | Site | 0.710 |
| C) NTX 0.01 mg | 51 | −0.06 | 0.420 | −1 | 1 | Treatment by Site | 0.676 |
| D) MS 60 mg/NTX 0.001 mg | 50 | −0.04 | 0.402 | −1 | 1 | A-B | N/D |
| E) MS 60 mg/NTX 0.01 mg | 51 | −0.06 | 0.544 | −1 | 2 | A-C | N/D |
| F) MS 60 mg/NTX 0.1 mg | 48 | 0.02 | 0.483 | −1 | 2 | A-D | N/D |
| | | | | | | A-E | N/D |
| | | | | | | A-F | N/D |
| | | | | | | B-C | N/D |
| | | | | | | B-D | N/D |
| | | | | | | B-E | N/D |
| | | | | | | B-F | N/D |
| | | | | | | C-D | N/D |
| | | | | | | C-E | N/D |
| | | | | | | C-F | N/D |
| | | | | | | D-E | N/D |
| | | | | | | D-F | N/D |
| | | | | | | E-F | N/D |
| 30 MINUTES | | | | | | | |
| A) Placebo | 51 | −0.02 | 0.424 | −1 | 1 | Treatment | 0.350 |
| B) MS 60 mg | 53 | −0.08 | 0.474 | −1 | 1 | Site | 0.710 |
| C) NTX 0.01 mg | 51 | −0.18 | 0.590 | −1 | 1 | Treatment by Site | 0.566 |
| D) MS 60 mg/NTX 0.001 mg | 50 | −0.10 | 0.544 | −1 | 1 | A-B | N/D |
| E) MS 60 mg/NTX 0.01 mg | 51 | −0.08 | 0.744 | −1 | 3 | A-C | N/D |
| F) MS 60 mg/NTX 0.1 mg | 48 | 0.06 | 0.522 | −1 | 2 | A-D | N/D |
| | | | | | | A-E | N/D |
| | | | | | | A-F | N/D |
| | | | | | | B-C | N/D |
| | | | | | | B-D | N/D |
| | | | | | | B-E | N/D |
| | | | | | | B-F | N/D |
| | | | | | | C-D | N/D |
| | | | | | | C-E | N/D |
| | | | | | | C-F | N/D |
| | | | | | | D-E | N/D |
| | | | | | | D-F | N/D |
| | | | | | | E-F | N/D |
| 45 MINUTES | | | | | | | |
| A) Placebo | 51 | −0.08 | 0.523 | −1 | 1 | Treatment | 0.067 |
| B) MS 60 mg | 53 | 0.00 | 0.650 | −1 | 2 | Site | 0.632 |
| C) NTX 0.01 mg | 51 | −0.22 | 0.610 | −1 | 2 | Treatment by Site | 0.896 |
| D) MS 60 mg/NTX 0.001 mg | 50 | 0.06 | 0.793 | −1 | 2 | A-B | N/D |
| E) MS 60 mg/NTX 0.01 mg | 51 | 0.22 | 0.945 | −1 | 3 | A-C | N/D |
| F) MS 60 mg/NTX 0.1 mg | 48 | 0.17 | 0.724 | −1 | 3 | A-D | N/D |
| | | | | | | A-E | N/D |
| | | | | | | A-F | N/D |
| | | | | | | B-C | N/D |
| | | | | | | B-D | N/D |
| | | | | | | B-E | N/D |
| | | | | | | B-F | N/D |
| | | | | | | C-D | N/D |
| | | | | | | C-E | N/D |
| | | | | | | C-F | N/D |
| | | | | | | D-E | N/D |
| | | | | | | D-F | N/D |
| | | | | | | E-F | N/D |
| 1 HOUR | | | | | | | |
| A) Placebo | 51 | −0.10 | 0.539 | −1 | 1 | Treatment | 0.023* |
| B) MS 60 mg | 53 | 0.17 | 0.727 | −1 | 2 | Site | 0.560 |
| C) NTX 0.01 mg | 51 | −0.12 | 0.739 | −1 | 2 | Treatment by Site | 0.798 |
| D) MS 60 mg/NTX 0.001 mg | 50 | 0.16 | 0.866 | −1 | 3 | A-B | 0.098 |
| E) MS 60 mg/NTX 0.01 mg | 51 | 0.27 | 0.896 | −1 | 3 | A-C | 0.842 |
| F) MS 60 mg/NTX 0.1 mg | 48 | 0.35 | 0.812 | −1 | 3 | A-D | 0.159 |
| | | | | | | A-E | 0.031* |
| | | | | | | A-F | 0.008** |
| | | | | | | B-C | 0.065 |
| | | | | | | B-D | 0.827 |
| | | | | | | B-E | 0.599 |
| | | | | | | B-F | 0.296 |
| | | | | | | C-D | 0.110 |
| | | | | | | C-E | 0.019* |
| | | | | | | C-F | 0.004** |
| | | | | | | D-E | 0.464 |
| | | | | | | D-F | 0.216 |
| | | | | | | E-F | 0.598 |
| 1.5 HOURS | | | | | | | |
| A) Placebo | 51 | −0.08 | 0.627 | −1 | 2 | Treatment | 0.010* |
| B) MS 60 mg | 53 | 0.28 | 0.744 | −1 | 2 | Site | 0.497 |
| C) NTX 0.01 mg | 51 | −0.10 | 0.700 | −1 | 2 | Treatment by Site | 0.617 |
| D) MS 60 mg/NTX 0.001 mg | 50 | 0.20 | 0.948 | −1 | 3 | A-B | 0.038* |
| E) MS 60 mg/NTX 0.01 mg | 51 | 0.35 | 0.890 | −1 | 3 | A-C | 0.853 |
| F) MS 60 mg/NTX 0.1 mg | 48 | 0.42 | 0.871 | −1 | 3 | A-D | 0.126 |
| | | | | | | A-E | 0.015* |
| | | | | | | A-F | 0.008** |
| | | | | | | B-C | 0.024* |
| | | | | | | B-D | 0.609 |
| | | | | | | B-E | 0.707 |
| | | | | | | B-F | 0.519 |
| | | | | | | C-D | 0.088 |
| | | | | | | C-E | 0.009** |
| | | | | | | C-F | 0.004** |
| | | | | | | D-E | 0.381 |
| | | | | | | D-F | 0.258 |
| | | | | | | E-F | 0.783 |
| 2 HOURS | | | | | | | |
| A) Placebo | 51 | −0.12 | 0.683 | −1 | 2 | Treatment | <.001*** |
| B) MS 60 mg | 53 | 0.30 | 0.868 | −1 | 2 | Site | 0.290 |
| C) NTX 0.01 mg | 51 | −0.16 | 0.674 | −1 | 2 | Treatment by Site | 0.489 |
| D) MS 60 mg/NTX 0.001 mg | 50 | 0.26 | 0.965 | −1 | 3 | A-B | 0.019* |
| E) MS 60 mg/NTX 0.01 mg | 51 | 0.31 | 0.883 | −1 | 3 | A-C | 0.817 |
| F) MS 60 mg/NTX 0.1 mg | 48 | 0.58 | 0.964 | −1 | 3 | A-D | 0.039* |
| | | | | | | A-E | 0.016* |
| | | | | | | A-F | <.001*** |
| | | | | | | B-C | 0.010* |
| | | | | | | B-D | 0.813 |
| | | | | | | B-E | 0.946 |
| | | | | | | B-F | 0.170 |
| | | | | | | C-D | 0.022* |
| | | | | | | C-E | 0.009** |
| | | | | | | C-F | <.001*** |
| | | | | | | D-E | 0.763 |
| | | | | | | D-F | 0.114 |
| | | | | | | E-F | 0.194 |
| 3 HOURS | | | | | | | |
| A) Placebo | 51 | −0.06 | 0.785 | −1 | 2 | Treatment | <.001*** |
| B) MS 60 mg | 53 | 0.27 | 0.858 | −1 | 2 | Site | 0.168 |
| C) NTX 0.01 mg | 51 | −0.18 | 0.684 | −1 | 2 | Treatment by Site | 0.526 |
| D) MS 60 mg/NTX 0.001 mg | 50 | 0.36 | 1.064 | −1 | 3 | A-B | 0.087 |
| E) MS 60 mg/NTX 0.01 mg | 51 | 0.43 | 0.964 | −1 | 3 | A-C | 0.504 |
| F) MS 60 mg/NTX 0.1 mg | 48 | 0.60 | 1.005 | −1 | 3 | A-D | 0.029* |
| | | | | | | A-E | 0.011* |
| | | | | | | A-F | 0.001** |
| | | | | | | B-C | 0.017* |
| | | | | | | B-D | 0.610 |
| | | | | | | B-E | 0.402 |
| | | | | | | B-F | 0.119 |
| | | | | | | C-D | 0.004** |
| | | | | | | C-E | 0.001** |
| | | | | | | C-F | <.001*** |
| | | | | | | D-E | 0.751 |
| | | | | | | D-F | 0.300 |
| | | | | | | E-F | 0.462 |
| 4 HOURS | | | | | | | |
| A) Placebo | 51 | 0.02 | 0.883 | −1 | 2 | Treatment | 0.001** |
| B) MS 60 mg | 53 | 0.36 | 0.963 | −1 | 3 | Site | 0.163 |
| C) NTX 0.01 mg | 51 | −0.18 | 0.684 | −1 | 2 | Treatment by Site | 0.414 |
| D) MS 60 mg/NTX 0.001 mg | 50 | 0.42 | 1.108 | −1 | 3 | A-B | 0.103 |
| E) MS 60 mg/NTX 0.01 mg | 51 | 0.43 | 0.964 | −1 | 3 | A-C | 0.298 |
| F) MS 60 mg/NTX 0.1 mg | 48 | 0.67 | 1.136 | −1 | 3 | A-D | 0.054 |
| | | | | | | A-E | 0.051 |
| | | | | | | A-F | 0.004** |
| | | | | | | B-C | 0.007** |
| | | | | | | B-D | 0.743 |
| | | | | | | B-E | 0.741 |
| | | | | | | B-F | 0.202 |
| | | | | | | C-D | 0.003** |
| | | | | | | C-E | 0.003** |
| | | | | | | C-F | <.001*** |
| | | | | | | D-E | 0.997 |
| | | | | | | D-F | 0.350 |
| | | | | | | E-F | 0.343 |
| 5 HOURS | | | | | | | |
| A) Placebo | 51 | 0.02 | 0.883 | −1 | 2 | Treatment | 0.001** |
| B) MS 60 mg | 53 | 0.32 | 0.936 | −1 | 2 | Site | 0.058 |
| C) NTX 0.01 mg | 51 | −0.16 | 0.674 | −1 | 2 | Treatment by Site | 0.174 |
| D) MS 60 mg/NTX 0.001 mg | 50 | 0.46 | 1.129 | −1 | 3 | A-B | 0.141 |
| E) MS 60 mg/NTX 0.01 mg | 51 | 0.43 | 1.005 | −1 | 3 | A-C | 0.355 |
| F) MS 60 mg/NTX 0.1 mg | 48 | 0.65 | 1.120 | −1 | 3 | A-D | 0.029* |
| | | | | | | A-E | 0.046* |
| | | | | | | A-F | 0.007** |
| | | | | | | B-C | 0.017* |
| | | | | | | B-D | 0.452 |
| | | | | | | B-E | 0.591 |
| | | | | | | B-F | 0.209 |
| | | | | | | C-D | 0.002** |
| | | | | | | C-E | 0.003** |
| | | | | | | C-F | <.001*** |
| | | | | | | D-E | 0.826 |
| | | | | | | D-F | 0.615 |
| | | | | | | E-F | 0.467 |
| 6 HOURS | | | | | | | |
| A) Placebo | 51 | 0.02 | 0.905 | −1 | 3 | Treatment | 0.005** |
| B) MS 60 mg | 53 | 0.23 | 0.869 | −1 | 2 | Site | 0.019* |
| C) NTX 0.01 mg | 51 | −0.16 | 0.674 | −1 | 2 | Treatment by Site | 0.191 |
| D) MS 60 mg/NTX 0.001 mg | 50 | 0.38 | 1.086 | −1 | 3 | A-B | 0.302 |
| E) MS 60 mg/NTX 0.01 mg | 51 | 0.41 | 1.062 | −1 | 3 | A-C | 0.367 |
| F) MS 60 mg/NTX 0.1 mg | 48 | 0.60 | 1.086 | −1 | 3 | A-D | 0.077 |
| | | | | | | A-E | 0.053 |
| | | | | | | A-F | 0.011* |
| | | | | | | B-C | 0.053 |
| | | | | | | B-D | 0.448 |
| | | | | | | B-E | 0.359 |
| | | | | | | B-F | 0.124 |
| | | | | | | C-D | 0.008** |
| | | | | | | C-E | 0.004** |
| | | | | | | C-F | <.001*** |
| | | | | | | D-E | 0.883 |
| | | | | | | D-F | 0.439 |
| | | | | | | E-F | 0.525 |
| 7 HOURS | | | | | | | |
| A) Placebo | 51 | 0.00 | 0.872 | −1 | 3 | Treatment | 0.002** |
| B) MS 60 mg | 53 | 0.21 | 0.885 | −1 | 2 | Site | 0.025* |
| C) NTX 0.01 mg | 51 | −0.16 | 0.674 | −1 | 2 | Treatment by Site | 0.361 |
| D) MS 60 mg/NTX 0.001 mg | 50 | 0.36 | 1.064 | −1 | 3 | A-B | 0.287 |
| E) MS 60 mg/NTX 0.01 mg | 51 | 0.45 | 1.101 | −1 | 3 | A-C | 0.442 |
| F) MS 60 mg/NTX 0.1 mg | 48 | 0.65 | 1.120 | −1 | 3 | A-D | 0.083 |
| | | | | | | A-E | 0.025* |
| | | | | | | A-F | 0.004** |
| | | | | | | B-C | 0.067 |
| | | | | | | B-D | 0.487 |
| | | | | | | B-E | 0.230 |
| | | | | | | B-F | 0.061 |
| | | | | | | C-D | 0.013* |
| | | | | | | C-E | 0.002** |
| | | | | | | C-F | <.001*** |
| | | | | | | D-E | 0.625 |
| | | | | | | D-F | 0.245 |
| | | | | | | E-F | 0.490 |
| 8 HOURS | | | | | | | |
| A) Placebo | 51 | 0.00 | 0.872 | −1 | 3 | Treatment | 0.002** |
| B) MS 60 mg | 53 | 0.21 | 0.906 | −1 | 2 | Site | 0.039* |
| C) NTX 0.01 mg | 51 | −0.16 | 0.674 | −1 | 2 | Treatment by Site | 0.365 |
| D) MS 60 mg/NTX 0.001 mg | 50 | 0.36 | 1.064 | −1 | 3 | A-B | 0.304 |
| E) MS 60 mg/NTX 0.01 mg | 51 | 0.45 | 1.101 | −1 | 3 | A-C | 0.420 |
| F) MS 60 mg/NTX 0.1 mg | 48 | 0.63 | 1.084 | −1 | 3 | A-D | 0.089 |
| | | | | | | A-E | 0.027* |
| | | | | | | A-F | 0.005** |
| | | | | | | B-C | 0.067 |
| | | | | | | B-D | 0.486 |
| | | | | | | B-E | 0.229 |
| | | | | | | B-F | 0.074 |
| | | | | | | C-D | 0.013* |
| | | | | | | C-E | 0.002** |
| | | | | | | C-F | <.001*** |
| | | | | | | D-E | 0.625 |
| | | | | | | D-F | 0.282 |
| | | | | | | E-F | 0.546 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, or <=0.001 RESPECTIVELY.
N/D: NOTE DONE (BECAUSE OVERALL P-VALUE NOT SIGNIFICANT).

Tables 37A and 37B present the mean MAXPAR and PEAKPID scores. The mean MAXPAR scores presented in Table 37A varied among treatment groups. The mean MAXPAR score was highest for the 0.1 mg NTX combination treatment group compared to all other groups. The mean scores for the 0.01 mg NTX and 0.001 mg NTX combination treatment groups were comparable to the mean score for the MS alone treatment group, which in turn, was greater than the mean score for the placebo and the 0.01 mg NTX alone treatment groups. The mean PEAKPID scores presented in Table 37B varied among treatment groups, and were greater for the MS alone or NTX combination treatment groups compared to the placebo and the 0.01 mg NTX alone treatment groups. Compared to all other groups, the mean PEAKPID scores were highest for the 0.1 mg NTX combination treatment group.

TABLE 37A
Maximum Pain Relief Scores (MAXPAR)
Intent-To-Treat Population, All Patients

| | MAXIMUM PAIN RELIEF SCORE [1] | | | | | | | |
|---|---|---|---|---|---|---|---|---|
| TREATMENT | N | MEAN | SD | MIN | MEDIAN | MAX | SOURCE | P-VALUE [2] |
| A) Placebo | 51 | 0.86 | 1.167 | 0 | 0.00 | 4 | TREATMENT | <.001*** |
| B) MS 60 mg | 53 | 1.64 | 1.257 | 0 | 1.00 | 4 | SITE | 0.663 |
| C) NTX 0.01 mg | 51 | 0.63 | 0.894 | 0 | 0.00 | 3 | TREATMENT BY SITE | 0.321 |
| D) MS 60 mg/NTX 0.001 mg | 50 | 1.54 | 1.460 | 0 | 1.00 | 4 | A-B | 0.004** |
| E) MS 60 mg/NTX 0.01 mg | 51 | 1.61 | 1.471 | 0 | 2.00 | 4 | A-C | 0.337 |
| F) MS 60 mg/NTX 0.1 mg | 48 | 2.06 | 1.405 | 0 | 2.00 | 4 | A-D | 0.010* |
| | | | | | | | A-E | 0.007** |
| | | | | | | | A-F | <.001*** |
| | | | | | | | B-C | <.001*** |
| | | | | | | | B-D | 0.789 |
| | | | | | | | B-E | 0.847 |
| | | | | | | | B-F | 0.194 |
| | | | | | | | C-D | <.001*** |
| | | | | | | | C-E | <.001*** |
| | | | | | | | C-F | <.001*** |
| | | | | | | | D-E | 0.938 |
| | | | | | | | D-F | 0.125 |
| | | | | | | | E-F | 0.140 |

[1] PAIN RELIEF (PR) SCORES: 0 = NONE, 1 = A LITTLE, 2 = SOME, 3 = A LOT, 4 = COMPLETE.
[2] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, OR <=0.001 RESPECTIVELY.

TABLE 37B
Peak Pain Intensity Differences (PEAKPID)
Intent-To-Treat Population, All Patients

| PEAK PAIN INTENSITY DIFFERENCES (PEAKPID) | | | | | | | | |
|---|---|---|---|---|---|---|---|---|
| TREATMENT | N | MEAN | SD | MIN | MEDIAN | MAX | SOURCE | P-VALUE [1] |
| A) Placebo | 51 | 0.35 | 0.820 | −1 | 0.00 | 3 | TREATMENT | 0.001** |
| B) MS 60 mg | 53 | 0.64 | 0.901 | −1 | 0.00 | 3 | SITE | 0.187 |
| C) NTX 0.01 mg | 51 | 0.16 | 0.612 | −1 | 0.00 | 2 | TREATMENT BY SITE | 0.307 |
| D) MS 60 mg/NTX 0.001 mg | 50 | 0.72 | 0.927 | −1 | 0.00 | 3 | A-B | 0.137 |
| E) MS 60 mg/NTX 0.01 mg | 51 | 0.71 | 1.064 | −1 | 0.00 | 3 | A-C | 0.252 |
| F) MS 60 mg/NTX 0.1 mg | 48 | 0.96 | 0.988 | −1 | 1.00 | 3 | A-D | 0.069 |
| | | | | | | | A-E | 0.096 |
| | | | | | | | A-F | 0.004** |
| | | | | | | | B-C | 0.008** |
| | | | | | | | B-D | 0.718 |
| | | | | | | | B-E | 0.850 |
| | | | | | | | B-F | 0.147 |
| | | | | | | | C-D | 0.003** |
| | | | | | | | C-E | 0.005** |
| | | | | | | | C-F | <.001*** |
| | | | | | | | D-E | 0.862 |
| | | | | | | | D-F | 0.283 |
| | | | | | | | E-F | 0.209 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, OR <=0.001 RESPECTIVELY.

Table 38 presents the summary and analysis of global evaluations. The NTX alone and placebo treatment groups had the highest number of subjects who had “poor” global evaluation scores. The profiles of the global evaluations scores are based on subjects' evaluations.

TABLE 38
Global Evaluation of Study Medication
Intent-To-Treat Population, All Patients

| | | | | | VERY | | | | | |
|---|---|---|---|---|---|---|---|---|---|---|
| | | POOR | FAIR | GOOD | GOOD | EXCELLENT | | | | P-VALUE |
| TREATMENT | N | (0) | (1) | (2) | (3) | (4) | MEAN | (SD) | SOURCE | [1] |
| A) Placebo | 51 | 40 (78.4%) | 4 (7.8%) | 5 (9.8%) | 2 (3.9%) | 0 (0.0%) | 0.4 | 0.83 | Treatment | <.001*** |
| B) MS 60 mg | 52 | 25 (48.1%) | 7 (13.5%) | 11 (21.2%) | 7 (13.5%) | 2 (3.8%) | 1.1 | 1.26 | A-B | 0.001** |
| C) NTX 0.01 mg | 50 | 45 (90.0%) | 3 (6.0%) | 0 (0.0%) | 1 (2.0%) | 1 (2.0%) | 0.2 | 0.73 | A-C | 0.222 |
| D) MS 60 mg/NTX 0.001 mg | 47 | 26 (55.3%) | 6 (12.8%) | 5 (10.6%) | 7 (14.9%) | 3 (6.4%) | 1.0 | 1.37 | A-D | 0.006** |
| E) MS 60 mg/NTX 0.01 mg | 50 | 21 (42.0%) | 9 (18.0%) | 4 (8.0%) | 11 (22.0%) | 5 (10.0%) | 1.4 | 1.47 | A-E | <.001*** |
| F) MS 60 mg/NTX 0.1 mg | 48 | 17 (35.4%) | 10 (20.8%) | 5 (10.4%) | 10 (20.8%) | 6 (12.5%) | 1.5 | 1.47 | A-F | <.001*** |
| | | | | | | | | | B-C | <.001*** |
| | | | | | | | | | B-D | 0.770 |
| | | | | | | | | | B-E | 0.287 |
| | | | | | | | | | B-F | 0.114 |
| | | | | | | | | | C-D | <.001*** |
| | | | | | | | | | C-E | <.001*** |
| | | | | | | | | | C-F | <.001*** |
| | | | | | | | | | D-E | 0.195 |
| | | | | | | | | | D-F | 0.072 |
| | | | | | | | | | E-F | 0.661 |

[1] FROM COCHRAN-MANTEL-HAENZEL TEST FOR RAW MEAN SCORES DIFFERENCE, ADJUSTING FOR SITE.
*, **, ***P-VALUE <=0.05, <=0.01, OR <=0.001 RESPECTIVELY.

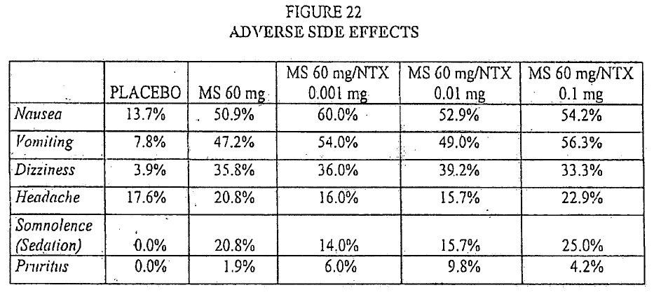

The majority of adverse events reported were categorized as digestive (nausea or vomiting) or nervous system (dizziness or somnolence) as further shown in Table 39A and 39B. FIG. 22 represents a summary of exemplary adverse side effects that may be attenuated according to methods and compositions of the invention.

TABLE 39A
ADVERSE EVENTS BY BODY SYSTEM AND SEVERITY
INTENT-TO-TREAT POPULATION, ALL PATIENTS

| | | Total | No. of | | | | | | |
|---|---|---|---|---|---|---|---|---|---|
| Body System | | No. of | Patients | | P-Value | No. of | SEVERITY [2] | | |
| Adverse Events | Treatment | Patients | w/Event | Source | [1] | Events | Mild | Moderate | Severe |

ALL BODY SYSTEMS

| | | | | | | | | | |
|---|---|---|---|---|---|---|---|---|---|
| All EVENTS | A) PLACEBO | 51 | 29 (56.9%) | Treatment | <.001*** | 53 | 18 (34.0%) | 19 (35.8%) | 16 (30.2%) |
| | B) MS 60 mg | 53 | 46 (86.8%) | A-B | <.001*** | 175 | 62 (35.4%) | 77 (44.0%) | 36 (20.6%) |
| | C) NTX 0.01 mg | 51 | 28 (54.9%) | A-D | <.001*** | 61 | 17 (27.9%) | 27 (44.3%) | 17 (27.9%) |
| | D) MS 60 mg/NTX 0.001 mg | 50 | 46 (92.0%) | A-E | <.001*** | 141 | 47 (33.3%) | 58 (41.1%) | 36 (25.5%) |
| | E) MS 60 mg/NTX 0.01 mg | 51 | 48 (94.1%) | A-F | <.001*** | 161 | 53 (32.9%) | 58 (36.0%) | 50 (31.1%) |
| | F) MS 60 mg/NTX 0.1 mg | 48 | 44 (91.7%) | B-C | <.001*** | 143 | 43 (30.1%) | 61 (42.7%) | 39 (27.3%) |
| | | | | C-D | <.001*** | | | | |
| | | | | C-E | <.001*** | | | | |
| | | | | C-F | <.001*** | | | | |

CARDIAC DISORDERS

| | | | | | | | | | |
|---|---|---|---|---|---|---|---|---|---|
| ALL EVENTS | A) PLACEBO | 51 | 1 (2.0%) | Treatment | 0.785 | 1 | 1 (100.0%) | 0 | 0 |
| | B) MS 60 mg | 53 | 2 (3.8%) | | | 2 | 2 (100.0%) | 0 | 0 |
| | C) NTX 0.01 mg | 51 | 2 (3.9%) | | | 2 | 1 (50.0%) | 1 (50.0%) | 0 |
| | D) MS 60 mg/NTX 0.001 mg | 50 | 2 (4.0%) | | | 2 | 1 (50.0%) | 1 (50.0%) | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| BRADY- | A) PLACEBO | 51 | 1 (2.0%) | Treatment | 0.418 | 1 | 1 (100.0%) | 0 | 0 |
| CARDIA NOS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| PALPITATIONS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| TACHYCARDIA | A) PLACEBO | 51 | 0 | Treatment | 0.309 | 0 | 0 | 0 | 0 |
| NOS | B) MS 60 mg | 53 | 2 (3.8%) | | | 2 | 2 (100.0%) | 0 | 0 |
| | C) NTX 0.01 mg | 51 | 2 (3.9%) | | | 2 | 1 (50.0%) | 1 (50.0%) | 0 |
| | D) MS 60 mg/NTX 0.001 mg | 50 | 2 (4.0%) | | | 2 | 1 (50.0%) | 1 (50.0%) | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

EAR AND LABYRINTH DISORDERS

| | | | | | | | | | |
|---|---|---|---|---|---|---|---|---|---|
| ALL EVENTS | A) PLACEBO | 51 | 3 (5.9%) | Treatment | 0.305 | 4 | 2 (50.0%) | 2 (50.0%) | 0 |
| | B) MS 60 mg | 53 | 1 (1.9%) | E-F | 0.047* | 1 | 1 (100.0%) | 0 | 0 |
| | C) NTX 0.01 mg | 51 | 2 (3.9%) | | | 2 | 0 | 2 (100.0%) | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 4 (7.8%) | | | 4 | 0 | 4 (100.0%) | 0 |
| | F) MS 60 mg/NTX 0.1 mg | 48 | 0 | | | 0 | 0 | 0 | 0 |
| EARACHE | A) PLACEBO | 51 | 3 (5.9%) | Treatment | 0.265 | 4 | 2 (50.0%) | 2 (50.0%) | 0 |
| | B) MS 60 mg | 53 | 0 | | | 0 | 0 | 0 | 0 |
| | C) NTX 0.01 mg | 51 | 2 (3.9%) | | | 2 | 0 | 2 (100.0%) | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 3 (5.9%) | | | 3 | 0 | 3 (100.0%) | 0 |
| | F) MS 60 mg/NTX 0.1 mg | 48 | 0 | | | 0 | 0 | 0 | 0 |
| HEARING | A) PLACEBO | 51 | 0 | Treatment | 0.418 | 0 | 0 | 0 | 0 |
| IMPAIRED | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 1 (100.0%) | 0 |
| | F) MS 60 mg/NTX 0.1 mg | 48 | 0 | | | 0 | 0 | 0 | 0 |
| HYPERACUSIS | A) PLACEBO | 51 | 0 | Treatment | 0.446 | 0 | 0 | 0 | 0 |
| | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

EYE DISORDERS

| | | | | | | | | | |
|---|---|---|---|---|---|---|---|---|---|
| ALL EVENTS | A) PLACEBO | 51 | 1 (2.0%) | Treatment | 0.017* | 1 | 0 | 1 (100.0%) | 0 |
| | B) MS 60 mg | 53 | 10 (18.9%) | A-B | 0.005** | 10 | 7 (70.0%) | 2 (20.0%) | 1 (10.0%) |
| | C) NTX 0.01 mg | 51 | 1 (2.0%) | A-D | 0.047* | 1 | 0 | 1 (100.0%) | 0 |
| | D) MS 60 mg/NTX 0.001 mg | 50 | 6 (12.0%) | B-C | 0.005** | 6 | 5 (83.3%) | 0 | 1 (16.7%) |
| | E) MS 60 mg/NTX 0.01 mg | 51 | 4 (7.8%) | C-D | 0.047 | 4 | 3 (75.0%) | 0 | 1 (25.0%) |
| | F) MS 60 mg/NTX 0.1 mg | 48 | 4 (8.3%) | | | 4 | 4 (100.0%) | 0 | 0 |
| AMBLYOPIA | A) PLACEBO | 51 | 0 | Treatment | 0.374 | 0 | 0 | 0 | 0 |
| NOS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 1 (100.0%) | 0 | 0 |
| CONJUNC- | A) PLACEBO | 51 | 0 | Treatment | 0.068 | 0 | 0 | 0 | 0 |
| TIVITIS | B) MS 60 mg | 53 | 7 (13.2%) | A-B | 0.007** | 7 | 6 (85.7%) | 1 (14.3%) | 0 |
| NEC | C) NTX 0.01 mg | 51 | 1 (2.0%) | A-D | 0.020* | 1 | 0 | 1 (100.0%) | 0 |
| | D) MS 60 mg/NTX 0.001 mg | 50 | 5 (10.0%) | A-E | 0.041* | 5 | 5 (100.0%) | 0 | 0 |
| | E) MS 60 mg/NTX 0.01 mg | 51 | 4 (7.8%) | B-C | 0.031* | 4 | 3 (75.0%) | 0 | 1 (25.0%) |
| | F) MS 60 mg/NTX 0.1 mg | 48 | 3 (6.3%) | | | 3 | 3 (100.0%) | 0 | 0 |
| PHOTOPHOBIA | A) PLACEBO | 51 | 1 (2.0%) | Treatment | 0.418 | 1 | 0 | 1 (100.0%) | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| RED EYE | A) PLACEBO | 51 | 0 | Treatment | 0.446 | 0 | 0 | 0 | 0 |
| | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| TIRED EYES | A) PLACEBO | 51 | 0 | Treatment | 0.404 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 0 | 0 | 1 (100.0%) |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| VISION | A) PLACEBO | 51 | 0 | Treatment | 0.089 | 0 | 0 | 0 | 0 |
| BLURRED | B) MS 60 mg | 53 | 2 (3.8%) | | | 2 | 1 (50.0%) | 1 (50.0%) | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

GASTROINTESTINAL DISORDERS

| | | | | | | | | | |
|---|---|---|---|---|---|---|---|---|---|
| ALL EVENTS | A) PLACEBO | 51 | 12 (23.5%) | Treatment | <.001*** | 16 | 4 (25.0%) | 4 (25.0%) | 8 (50.0%) |
| | B) MS 60 mg | 53 | 33 (62.3%) | A-B | <.001*** | 61 | 17 (27.9%) | 23 (37.7%) | 21 (34.4%) |
| | C) NTX 0.01 mg | 51 | 13 (25.5%) | A-D | <.001*** | 19 | 6 (31.6%) | 6 (31.6%) | 7 (36.8%) |
| | D) MS 60 mg/NTX 0.001 mg | 50 | 35 (70.0%) | A-E | <.001*** | 66 | 14 (21.2%) | 26 (39.4%) | 26 (39.4%) |
| | E) MS 60 mg/NTX 0.01 mg | 51 | 34 (66.7%) | A-F | <.001*** | 62 | 13 (21.0%) | 18 (29.0%) | 31 (50.0%) |
| | F) MS 60 mg/NTX 0.1 mg | 48 | 33 (68.8%) | B-C | <.001*** | 63 | 10 (15.9%) | 26 (41.3%) | 27 (42.9%) |
| | | | | C-D | <.001*** | | | | |
| | | | | C-E | <.001*** | | | | |
| | | | | C-F | <.001*** | | | | |
| ABDOMINAL | A) PLACEBO | 51 | 1 (2.0%) | Treatment | 0.439 | 1 | 0 | 0 | 1 (100.0%) |
| PAIN NOS | B) MS 60 mg | 53 | 2 (3.8%) | | | 2 | 1 (50.0%) | 1 (50.0%) | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| ABDOMINAL | A) PLACEBO | 51 | 0 | Treatment | 0.540 | 0 | 0 | 0 | 0 |
| PAIN UPPER | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 1 (100.0%) | 0 |
| DYSPEPSIA | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 1 (100.0%) | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| DYSPHAGIA | A) PLACEBO | 51 | 1 (2.0%) | Treatment | 0.208 | 1 | 0 | 0 | 1 (100.0%) |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 2 (4.0%) | | | 2 | 0 | 1 (50.0%) | 1 (50.0%) |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| HICCUPS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 1 (100.0%) | 0 |
| | F) MS 60 mg/NTX 0.1 mg | 48 | 0 | | | 0 | 0 | 0 | 0 |
| MELAENA | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 1 (100.0%) | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| NAUSEA | A) PLACEBO | 51 | 7 (13.7%) | Treatment | <.001*** | 8 | 3 (37.5%) | 2 (25.0%) | 3 (37.5%) |
| | B) MS 60 mg | 53 | 27 (50.9%) | A-B | <.001*** | 31 | 12 (38.7%) | 15 (48.4%) | 4 (12.9%) |
| | C) NTX 0.01 mg | 51 | 9 (17.6%) | A-D | <.001*** | 10 | 3 (30.0%) | 5 (50.0%) | 2 (20.0%) |
| | D) MS 60 mg/NTX 0.001 mg | 50 | 30 (60.0%) | A-E | <.001*** | 31 | 9 (29.0%) | 16 (51.6%) | 6 (19.4%) |
| | E) MS 60 mg/NTX 0.01 mg | 51 | 27 (52.9%) | A-F | <.001*** | 31 | 9 (29.0%) | 12 (38.7%) | 10 (32.3%) |
| | F) MS 60 mg/NTX 0.1 mg | 48 | 26 (54.2%) | B-C | <.001*** | 28 | 7 (25.0%) | 19 (67.9%) | 2 (7.1%) |
| | | | | C-D | <.001*** | | | | |
| | | | | C-E | <.001*** | | | | |
| | | | | C-F | <.001*** | | | | |
| ORAL PAIN | A) PLACEBO | 51 | 0 | Treatment | 0.214 | 0 | 0 | 0 | 0 |
| | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 2 (4.0%) | | | 2 | 0 | 0 | 2 (100.0%) |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| SORE THROAT | A) PLACEBO | 51 | 2 (3.9%) | Treatment | 0.217 | 2 | 0 | 2 (100.0%) | 0 |
| NOS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| STOMATITIS | A) PLACEBO | 51 | 0 | Treatment | 0.524 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 1 (2.1%) | | | 1 | 0 | 0 | 1 (100.0%) |
| VOMITING | A) PLACEBO | 51 | 4 (7.8%) | Treatment | <.001*** | 4 | 1 (25.0%) | 0 | 3 (75.0%) |
| NOS | B) MS 60 mg | 53 | 25 (47.2%) | A-B | <.001*** | 26 | 4 (15.4%) | 7 (26.9%) | 15 (57.7%) |
| | C) NTX 0.01 mg | 51 | 7 (13.7%) | A-D | <.001*** | 7 | 1 (14.3%) | 1 (14.3%) | 5 (71.4%) |
| | D) MS 60 mg/NTX 0.001 mg | 50 | 27 (54.0%) | A-E | <.001*** | 29 | 3 (10.3%) | 9 (31.0%) | 17 (58.6%) |
| | E) MS 60 mg/NTX 0.01 mg | 51 | 25 (49.0%) | A-F | <.001*** | 29 | 4 (13.8%) | 5 (17.2%) | 20 (69.0%) |
| | F) MS 60 mg/NTX 0.1 mg | 48 | 27 (56.3%) | B-C | <.001*** | 33 | 3 (9.1%) | 6 (18.2%) | 24 (72.7%) |
| | | | | C-D | <.001*** | | | | |
| | | | | C-E | <.001*** | | | | |
| | | | | C-F | <.001*** | | | | |

GENERAL DISORDERS AND ADMINISTRATION SITE CONDITIONS

| | | | | | | | | | |
|---|---|---|---|---|---|---|---|---|---|
| ALL EVENTS | A) PLACEBO | 51 | 5 (9.8%) | Treatment | 0.139 | 5 | 2 (40.0%) | 2 (40.0%) | 1 (20.0%) |
| | B) MS 60 mg | 53 | 13 (24.5%) | A-B | 0.047* | 13 | 5 (38.5%) | 7 (53.8%) | 1 (7.7%) |
| | C) NTX 0.01 mg | 51 | 4 (7.8%) | B-C | 0.021* | 5 | 1 (20.0%) | 2 (40.0%) | 2 (40.0%) |
| | D) MS 60 mg/NTX 0.001 mg | 50 | 7 (14.0%) | B-E | 0.047* | 7 | 4 (57.1%) | 3 (42.9%) | 0 |
| | E) MS 60 mg/NTX 0.01 mg | 51 | 5 (9.8%) | | | 8 | 4 (50.0%) | 2 (25.0%) | 2 (25.0%) |
| | F) MS 60 mg/NTX 0.1 mg | 48 | 6 (12.5%) | | | 6 | 4 (66.7%) | 2 (33.3%) | 0 |
| ASTHENIA | A) PLACEBO | 51 | 0 | Treatment | 0.001** | 0 | 0 | 0 | 0 |
| | B) MS 60 mg | 53 | 6 (11.3%) | A-B | 0.013* | 6 | 3 (50.0%) | 3 (50.0%) | 0 |
| | C) NTX 0.01 mg | 51 | 0 | B-C | 0.013* | 0 | 0 | 0 | 0 |
| | D) MS 60 mg/NTX 0.001 mg | 50 | 1 (2.0%) | B-F | 0.016* | 1 | 0 | 1 (100.0%) | 0 |
| | E) MS 60 mg/NTX 0.01 mg | 51 | 1 (2.0%) | | | 2 | 1 (50.0%) | 0 | 1 (50.0%) |
| | F) MS 60 mg/NTX 0.1 mg | 48 | 0 | | | 0 | 0 | 0 | 0 |
| FATIGUE | A) PLACEBO | 51 | 0 | Treatment | 0.446 | 0 | 0 | 0 | 0 |
| | B) MS 60 mg | 53 | 1 (1.9%) | | | 1 | 0 | 1 (100.0%) | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| FEELING | A) PLACEBO | 51 | 0 | Treatment | 0.446 | 0 | 0 | 0 | 0 |
| ABNORMAL | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| FEELING | A) PLACEBO | 51 | 0 | Treatment | 0.542 | 0 | 0 | 0 | 0 |
| HOT | B) MS 60 mg | 53 | 0 | | | 0 | 0 | 0 | 0 |
| | C) NTX 0.01 mg | 51 | 1 (2.0%) | | | 1 | 0 | 0 | 1 (100.0%) |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| FEELING | A) PLACEBO | 51 | 0 | Treatment | 0.548 | 0 | 0 | 0 | 0 |
| JITTERY | B) MS 60 mg | 53 | 2 (3.8%) | | | 2 | 1 (50.0%) | 1 (50.0%) | 0 |
| | C) NTX 0.01 mg | 51 | 0 | | | 0 | 0 | 0 | 0 |
| | D) MS 60 mg/NTX 0.001 mg | 50 | 2 (4.0%) | | | 2 | 1 (50.0%) | 1 (50.0%) | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 1 (2.1%) | | | 1 | 0 | 1 (100.0%) | 0 |
| PAIN IN FACE | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |
| PAIN NOS | A) PLACEBO | 51 | 1 (2.0%) | Treatment | 0.960 | 1 | 0 | 0 | 1 (100.0%) |
| | B) MS 60 mg | 53 | 1 (1.9%) | | | 1 | 0 | 1 (100.0%) | 0 |
| | C) NTX 0.01 mg | 51 | 1 (2.0%) | | | 1 | 0 | 0 | 1 (100.0%) |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 1 (100.0%) | 0 |
| PYREXIA | A) PLACEBO | 51 | 2 (3.9%) | Treatment | 0.975 | 2 | 2 (100.0%) | 0 | 0 |
| | B) MS 60 mg | 53 | 2 (3.8%) | | | 2 | 1 (50.0%) | 1 (50.0%) | 0 |
| | C) NTX 0.01 mg | 51 | 1 (2.0%) | | | 1 | 1 (100.0%) | 0 | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 2 (3.9%) | | | 2 | 1 (50.0%) | 1 (50.0%) | 0 |
| | F) MS 60 mg/NTX 0.1 mg | 48 | 2 (4.2%) | | | 2 | 2 (100.0%) | 0 | 0 |
| RIGORS | A) PLACEBO | 51 | 2 (3.9%) | Treatment | 0.623 | 2 | 0 | 2 (100.0%) | 0 |
| | B) MS 60 mg | 53 | 0 | | | 0 | 0 | 0 | 0 |
| | C) NTX 0.01 mg | 51 | 1 (2.0%) | | | 1 | 0 | 1 (100.0%) | 0 |
| | D) MS 60 mg/NTX 0.001 mg | 50 | 1 (2.0%) | | | 1 | 0 | 1 (100.0%) | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 1 (100.0%) | 0 | 0 |
| SHIVERING | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 0 | 1 (100.0%) | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| WEAKNESS | A) PLACEBO | 51 | 0 | Treatment | 0.211 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 2 (3.9%) | | | 2 | 1 (50.0%) | 1 (50.0%) | 0 |
| | F) MS 60 mg/NTX 0.1 mg | 48 | 1 (2.1%) | | | 1 | 1 (100.0%) | 0 | 0 |

HEPATO-BILIARY DISORDERS

| | | | | | | | | | |
|---|---|---|---|---|---|---|---|---|---|
| ALL EVENTS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |
| CHOLE- | A) PLACEBO | 51 | 0 | Treatment | 0.418 | 0 | 0 | 0 | 0 |
| LITHIASIS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |

INFECTIONS AND INFESTATIONS

| | | | | | | | | | |
|---|---|---|---|---|---|---|---|---|---|
| ALL EVENTS | A) PLACEBO | 51 | 8 (15.7%) | Treatment | 0.606 | 10 | 4 (40.0%) | 1 (10.0%) | 5 (50.0%) |
| | B) MS 60 mg | 53 | 6 (11.3%) | | | 7 | 1 (14.3%) | 3 (42.9%) | 3 (42.9%) |
| | C) NTX 0.01 mg | 51 | 9 (17.6%) | | | 10 | 1 (10.0%) | 5 (50.0%) | 4 (40.0%) |
| | D) MS 60 mg/NTX 0.001 mg | 50 | 6 (12.0%) | | | 6 | 0 | 1 (16.7%) | 5 (83.3%) |
| | E) MS 60 mg/NTX 0.01 mg | 51 | 4 (7.8%) | | | 5 | 0 | 0 | 5 (100.0%) |
| | F) MS 60 mg/NTX 0.1 mg | 48 | 4 (8.3%) | | | 5 | 0 | 2 (40.0%) | 3 (60.0%) |
| CELLULITIS | A) PLACEBO | 51 | 0 | Treatment | 0.211 | 0 | 0 | 0 | 0 |
| | B) MS 60 mg | 53 | 0 | | | 0 | 0 | 0 | 0 |
| | C) NTX 0.01 mg | 51 | 2 (3.9%) | | | 2 | 0 | 0 | 2 (100.0%) |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 0 | 1 (100.0%) |
| DRY SOCKET | A) PLACEBO | 51 | 3 (5.9%) | Treatment | 0.848 | 3 | 0 | 1 (33.3%) | 2 (66.7%) |
| NOS | B) MS 60 mg | 53 | 3 (5.7%) | | | 3 | 0 | 1 (33.3%) | 2 (66.7%) |
| | C) NTX 0.01 mg | 51 | 4 (7.8%) | | | 4 | 0 | 3 (75.0%) | 1 (25.0%) |
| | D) MS 60 mg/NTX 0.001 mg | 50 | 4 (8.0%) | | | 4 | 0 | 0 | 4 (100.0%) |
| | E) MS 60 mg/NTX 0.01 mg | 51 | 3 (5.9%) | | | 3 | 0 | 0 | 3 (100.0%) |
| | F) MS 60 mg/NTX 0.1 mg | 48 | 1 (2.1%) | | | 2 | 0 | 0 | 2 (100.0%) |
| NASO- | A) PLACEBO | 51 | 0 | Treatment | 0.446 | 0 | 0 | 0 | 0 |
| PHARYNGITIS | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| ORAL | A) PLACEBO | 51 | 0 | Treatment | 0.542 | 0 | 0 | 0 | 0 |
| INFECTION | B) MS 60 mg | 53 | 0 | | | 0 | 0 | 0 | 0 |
| NEC | C) NTX 0.01 mg | 51 | 0 | | | 0 | 0 | 0 | 0 |
| | D) MS 60 mg/NTX 0.001 mg | 50 | 1 (2.0%) | | | 1 | 0 | 1 (100.0%) | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |
| PHARYNGITIS | A) PLACEBO | 51 | 4 (7.8%) | Treatment | 0.546 | 6 | 3 (50.0%) | 0 | 3 (50.0%) |
| NOS | B) MS 60 mg | 53 | 2 (3.8%) | | | 3 | 1 (33.3%) | 2 (66.7%) | 0 |
| | C) NTX 0.01 mg | 51 | 3 (5.9%) | | | 4 | 1 (25.0%) | 2 (50.0%) | 1 (25.0%) |
| | D) MS 60 mg/NTX 0.001 mg | 50 | 1 (2.0%) | | | 1 | 0 | 0 | 1 (100.0%) |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 1 (2.1%) | | | 1 | 0 | 1 (100.0%) | 0 |
| TOOTH | A) PLACEBO | 51 | 0 | Treatment | 0.374 | 0 | 0 | 0 | 0 |
| INFECTION | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 1 (100.0%) | 0 |
| UPPER | A) PLACEBO | 51 | 1 (2.0%) | Treatment | 0.418 | 1 | 1 (100.0%) | 0 | 0 |
| RESPIRATORY | B) MS 60 mg | 53 | 0 | | | 0 | 0 | 0 | 0 |
| TRACT | C) NTX 0.01 mg | 51 | 0 | | | 0 | 0 | 0 | 0 |
| INFECTION | D) MS 60 mg/NTX 0.001 mg | 50 | 0 | | | 0 | 0 | 0 | 0 |
| NOS | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

INJURY AND POISONING

| | | | | | | | | | |
|---|---|---|---|---|---|---|---|---|---|
| ALL EVENTS | A) PLACEBO | 51 | 1 (2.0%) | Treatment | 0.418 | 1 | 0 | 1 (100.0%) | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| HYPOTHERMIA | A) PLACEBO | 51 | 1 (2.0%) | Treatment | 0.418 | 1 | 0 | 1 (100.0%) | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

INVESTIGATIONS

| | | | | | | | | | |
|---|---|---|---|---|---|---|---|---|---|
| ALL EVENTS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| HAEMATURIA | A) PLACEBO | 51 | 0 | Treatment | 0.418 | 0 | 0 | 0 | 0 |
| PRESENT | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |

MUSCULOSKELETAL, CONNECTIVE TISSUE AND BONE DISORDERS

| | | | | | | | | | |
|---|---|---|---|---|---|---|---|---|---|
| ALL EVENTS | A) PLACEBO | 51 | 0 | Treatment | 0.068 | 0 | 0 | 0 | 0 |
| | B) MS 60 mg | 53 | 3 (5.7%) | | | 5 | 0 | 4 (80.0%) | 1 (20.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 2 (3.9%0 | | | 2 | 1 (50.0%) | 1 (50.0%) | 0 |
| | F) MS 60 mg/NTX 0.1 mg | 48 | 0 | | | 0 | 0 | 0 | 0 |
| JOINT | A) PLACEBO | 51 | 0 | Treatment | 0.418 | 0 | 0 | 0 | 0 |
| DISORDER NOS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 1 (100.0%) | 0 |
| | F) MS 60 mg/NTX 0.1 mg | 48 | 0 | | | 0 | 0 | 0 | 0 |
| MUSCLE | A) PLACEBO | 51 | 0 | Treatment | 0.418 | 0 | 0 | 0 | 0 |
| TWITCHING | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| MYALGIA | A) PLACEBO | 51 | 0 | Treatment | 0.446 | 0 | 0 | 0 | 0 |
| | B) MS 60 mg | 53 | 1 (1.9%) | | | 1 | 0 | 1 (100.0%) | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| NECK | A) PLACEBO | 51 | 0 | Treatment | 0.446 | 0 | 0 | 0 | 0 |
| STIFFNESS | B) MS 60 mg | 53 | 1 (1.9%) | | | 1 | 0 | 1 (100.0%) | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| SENSATION OF | A) PLACEBO | 51 | 0 | Treatment | 0.089 | 0 | 0 | 0 | 0 |
| HEAVINESS | B) MS 60 mg | 53 | 2 (3.8%) | | | 3 | 0 | 2 (66.7%) | 1 (33.3%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

NEOPLASMS BENIGN AND MALIGNANT (INCLUDING CYSTS AND POLYPS)

| | | | | | | | | | |
|---|---|---|---|---|---|---|---|---|---|
| ALL EVENTS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 0 | 0 | 1 (100.0%) |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| ADENOMA | A) PLACEBO | 51 | 0 | Treatment | 0.418 | 0 | 0 | 0 | 0 |
| BENIGN NOS | B) MS 60 mg | 53 | 0 | | | 0 | 0 | 0 | 0 |
| | C) NTX 0.01 mg | 51 | 1 (2.0%) | | | 1 | 0 | 0 | 1 (100.0%) |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

NERVOUS SYSTEM DISORDERS

| | | | | | | | | | |
|---|---|---|---|---|---|---|---|---|---|
| ALL EVENTS | A) PLACEBO | 51 | 13 (25.5%) | Treatment | <.001*** | 13 | 5 (38.5%) | 6 (46.2%) | 2 (15.4%) |
| | B) MS 60 mg | 53 | 33 (62.3%) | A-B | <.001*** | 52 | 12 (23.1%) | 34 (65.4%) | 6 (11.5%) |
| | C) NTX 0.01 mg | 51 | 14 (27.5%) | A-D | <.001*** | 15 | 5 (33.3%) | 8 (53.3%) | 2 (13.3%) |
| | D) MS 60 mg/NTX 0.001 mg | 50 | 31 (62.0%) | A-E | <.001*** | 40 | 16 (40.0%) | 21 (52.5%) | 3 (7.5%) |
| | E) MS 60 mg/NTX 0.01 mg | 51 | 33 (64.7%) | A-F | <.001*** | 50 | 21 (42.0%) | 23 (46.0%) | 6 (12.0%) |
| | F) MS 60 mg/NTX 0.1 mg | 48 | 30 (62.5%) | B-C | <.001*** | 45 | 19 (42.2%) | 20 (44.4%) | 6 (13.3%) |
| | | | | C-D | <.001*** | | | | |
| | | | | C-E | <.001*** | | | | |
| | | | | C-F | <.001*** | | | | |
| DIZZINESS | A) PLACEBO | 51 | 2 (3.9%) | Treatment | <.001*** | 2 | 0 | 2 (100.0%) | 0 |
| (EXC VERTIGO) | B) MS 60 mg | 53 | 19 (35.8%) | A-B | <.001*** | 21 | 4 (19.0%) | 14 (66.7%) | 3 (14.3%) |
| | C) NTX 0.01 mg | 51 | 2 (3.9%) | A-D | <.001*** | 2 | 2 (100.0%) | 0 | 0 |
| | D) MS 60 mg/NTX 0.001 mg | 50 | 18 (36.0%) | A-E | <.001*** | 19 | 7 (36.8%) | 11 (57.9%) | 1 (5.3%) |
| | E) MS 60 mg/NTX 0.01 mg | 51 | 20 (39.2%) | A-F | <.001*** | 23 | 10 (43.5%) | 12 (52.2%) | 1 (4.3%) |
| | F) MS 60 mg/NTX 0.1 mg | 48 | 16 (33.3%) | B-C | <.001*** | 19 | 7 (36.8%) | 9 (47.4%) | 3 (15.8%) |
| | | | | C-D | <.001*** | | | | |
| | | | | C-E | <.001*** | | | | |
| | | | | C-F | <.001*** | | | | |
| HEADACHE | A) PLACEBO | 51 | 9 (17.6%) | Treatment | 0.905 | 9 | 4 (44.4%) | 3 (33.3%) | 2 (22.2%) |
| NOS | B) MS 60 mg | 53 | 11 (20.8%) | | | 12 | 3 (25.0%) | 9 (75.0%) | 0 |
| | C) NTX 0.01 mg | 51 | 8 (15.7%) | | | 8 | 2 (25.0%) | 4 (50.0%) | 2 (25.0%) |
| | D) MS 60 mg/NTX 0.001 mg | 50 | 8 (16.0%) | | | 9 | 1 (11.1%) | 6 (66.7%) | 2 (22.2%) |
| | E) MS 60 mg/NTX 0.01 mg | 51 | 8 (15.7%) | | | 8 | 2 (25.0%) | 4 (50.0%) | 2 (25.0%) |
| | F) MS 60 mg/NTX 0.1 mg | 48 | 11 (22.9%) | | | 11 | 5 (45.5%) | 5 (45.5%) | 1 (9.1%) |
| HYPERTONIA | A) PLACEBO | 51 | 0 | Treatment | 0.551 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 0 | 1 (100.0%) | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| HYPO- | A) PLACEBO | 51 | 0 | Treatment | 0.418 | 0 | 0 | 0 | 0 |
| AESTHESIA | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| HYPOTONIA | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 1 (100.0%) | 0 |
| | F) MS 60 mg/NTX 0.1 mg | 48 | 0 | | | 0 | 0 | 0 | 0 |
| MIGRAINE NOS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 MG | 51 | 1 (2.0%) | | | 1 | 0 | 0 | 1 (100.0%) |
| | F) MS 60 mg/NTX 0.1 mg | 48 | 0 | | | 0 | 0 | 0 | 0 |
| MUSCLE | A) PLACEBO | 51 | 0 | Treatment | 0.446 | 0 | 0 | 0 | 0 |
| SPASTICITY | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| PARAESTHESIA | A) PLACEBO | 51 | 0 | Treatment | 0.404 | 0 | 0 | 0 | 0 |
| CIRCUMORAL | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| PARAESTHESIA | A) PLACEBO | 51 | 2 (3.9%) | Treatment | 0.993 | 2 | 1 (50.0%) | 1 (50.0%) | 0 |
| NEC | B) MS 60 mg | 53 | 3 (5.7%) | | | 5 | 2 (40.0%) | 2 (40.0%) | 1 (20.0%) |
| | C) NTX 0.01 mg | 51 | 3 (5.9%) | | | 3 | 1 (33.3%) | 2 (66.7%) | 0 |
| | D) MS 60 mg/NTX 0.001 mg | 50 | 3 (6.0%) | | | 3 | 3 (100.0%) | 0 | 0 |
| | E) MS 60 mg/NTX 0.01 mg | 51 | 3 (5.9%) | | | 3 | 2 (66.7%) | 1 (33.3%) | 0 |
| | F) MS 60 mg/NTX 0.1 mg | 48 | 2 (4.2%) | | | 2 | 1 (50.0%) | 1 (50.0%) | 0 |
| SOMNOLENCE | A) PLACEBO | 51 | 0 | Treatment | <.001*** | 0 | 0 | 0 | 0 |
| | B) MS 60 mg | 53 | 11 (20.8%) | A-B | <.001*** | 13 | 2 (15.4%) | 9 (69.2%) | 2 (15.4%) |
| | C) NTX 0.01 mg | 51 | 0 | A-D | 0.005** | 0 | 0 | 0 | 0 |
| | D) MS 60 mg/NTX 0.001 mg | 50 | 7 (14.0%) | A-E | 0.003** | 8 | 4 (50.0%) | 4 (50.0%) | 0 |
| | E) MS 60 mg/NTX 0.01 mg | 51 | 8 (15.7%) | A-F | <.001*** | 8 | 4 (50.0%) | 4 (50.0%) | 0 |
| | F) MS 60 mg/NTX 0.1 mg | 48 | 12 (25.0%) | B-C | <.001*** | 12 | 6 (50.0%) | 5 (41.7%) | 1 (8.3%) |
| | | | | C-D | 0.005** | | | | |
| | | | | C-E | 0.003** | | | | |
| | | | | C-F | <.001*** | | | | |
| SYNCOPE | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |
| TASTE LOSS | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 0 | 1 (100.0%) | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| TENSION | A) PLACEBO | 51 | 0 | Treatment | 0.374 | 0 | 0 | 0 | 0 |
| HEADACHES | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 0 | 1 (100.0%) |
| TREMOR NEC | A) PLACEBO | 51 | 0 | Treatment | 0.010* | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 3 (5.9%) | | | 3 | 1 (33.3%) | 1 (33.3%) | 1 (33.3%) |
| | F) MS 60 mg/NTX 0.1 mg | 48 | 0 | | | 0 | 0 | 0 | 0 |

PREGNANCY, PUERPERIUM AND PERINATAL CONDITIONS

| | | | | | | | | | |
|---|---|---|---|---|---|---|---|---|---|
| ALL EVENTS | A) PLACEBO | 51 | 0 | Treatment | 0.446 | 0 | 0 | 0 | 0 |
| | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| PREGNANCY | A) PLACEBO | 51 | 0 | Treatment | 0.446 | 0 | 0 | 0 | 0 |
| NOS | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |

PSYCHIATRIC DISORDERS

| | | | | | | | | | |
|---|---|---|---|---|---|---|---|---|---|
| ALL EVENTS | A) PLACEBO | 51 | 1 (2.0%) | Treatment | 0.179 | 1 | 0 | 1 (100.0%) | 0 |
| | B) MS 60 mg | 53 | 6 (11.3%) | | | 7 | 2 (28.6%) | 2 (28.6%) | 3 (42.9%) |
| | C) NTX 0.01 mg | 51 | 1 (2.0%) | | | 1 | 0 | 1 (100.0%) | 0 |
| | D) MS 60 mg/NTX 0.001 mg | 50 | 2 (4.0%) | | | 2 | 0 | 2 (100.0%) | 0 |
| | E) MS 60 mg/NTX 0.01 mg | 51 | 4 (7.8%) | | | 4 | 4 (100.0%) | 0 | 0 |
| | F) MS 60 mg/NTX 0.1 mg | 48 | 5 (10.4%) | | | 7 | 2 (28.6%) | 4 (57.1%) | 1 (14.3%) |
| ANXIETY NEC | A) PLACEBO | 51 | 0 | Treatment | 0.446 | 0 | 0 | 0 | 0 |
| | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| CONFUSION | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| DEPERSONALI- | A) PLACEBO | 51 | 0 | Treatment | 0.540 | 0 | 0 | 0 | 0 |
| SATION | B) MS 60 mg | 53 | 1 (1.9%) | | | 1 | 0 | 0 | 1 (100.0%) |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 1 (100.0%) | 0 | 0 |
| DIS- | A) PLACEBO | 51 | 0 | Treatment | 0.418 | 0 | 0 | 0 | 0 |
| ORIENTATION | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| DISSOCIATION | A) PLACEBO | 51 | 0 | Treatment | 0.056 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 2 (4.2%) | | | 2 | 0 | 1 (50.0%) | 1 (50.0%) |
| EUPHORIC | A) PLACEBO | 51 | 0 | Treatment | 0.130 | 0 | 0 | 0 | 0 |
| MOOD | B) MS 60 mg | 53 | 2 (3.8%) | | | 2 | 1 (50.0%) | 0 | 1 (50.0%) |
| | C) NTX 0.01 mg | 51 | 0 | | | 0 | 0 | 0 | 0 |
| | D) MS 60 mg/NTX 0.001 mg | 50 | 1 (2.0%) | | | 1 | 0 | 1 (100.0%) | 0 |
| | E) MS 60 mg/NTX 0.01 mg | 51 | 0 | | | 0 | 0 | 0 | 0 |
| | F) MS 60 mg/NTX 0.1 mg | 48 | 3 (6.3%) | | | 3 | 1 (33.3%) | 2 (66.7%) | 0 |
| NERVOUSNESS | A) PLACEBO | 51 | 1 (2.0%) | Treatment | 0.827 | 1 | 0 | 1 (100.0%) | 0 |
| | B) MS 60 mg | 53 | 3 (5.7%) | | | 3 | 1 (33.3%) | 2 (66.7%) | 0 |
| | C) NTX 0.01 mg | 51 | 1 (2.0%) | | | 1 | 0 | 1 (100.0%) | 0 |
| | D) MS 60 mg/NTX 0.001 mg | 50 | 1 (2.0%) | | | 1 | 0 | 1 (100.0%) | 0 |
| | E) MS 60 mg/NTX 0.01 mg | 51 | 2 (3.9%) | | | 2 | 2 (100.0%) | 0 | 0 |
| | F) MS 60 mg/NTX 0.1 mg | 48 | 1 (2.1%) | | | 1 | 0 | 1 (100.0%) | 0 |

RENAL AND URINARY DISORDERS

| | | | | | | | | | |
|---|---|---|---|---|---|---|---|---|---|
| ALL EVENTS | A) PLACEBO | 51 | 0 | Treatment | 0.226 | 0 | 0 | 0 | 0 |
| | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 2 (3.9%) | | | 2 | 0 | 2 (100.0%) | 0 |
| | F) MS 60 mg/NTX 0.1 mg | 48 | 0 | | | 0 | 0 | 0 | 0 |
| URINARY | A) PLACEBO | 51 | 0 | Treatment | 0.226 | 0 | 0 | 0 | 0 |
| RETENTION | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 2 (3.9%) | | | 2 | 0 | 2 (100.0%) | 0 |
| | F) MS 60 mg/NTX 0.1 mg | 48 | 0 | | | 0 | 0 | 0 | 0 |

REPRODUCTIVE SYSTEM AND BREAST DISORDERS

| | | | | | | | | | |
|---|---|---|---|---|---|---|---|---|---|
| ALL EVENTS | A) PLACEBO | 51 | 0 | Treatment | 0.542 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 0 | 0 | 1 (100.0%) |
| | E) MS 60 mg/NTX 0.01 mg | 51 | 1 (2.0%) | | | 2 | 0 | 1 (50.0%) | 1 (50.0%) |
| | F) MS 60 mg/NTX 0.1 mg | 48 | 0 | | | 0 | 0 | 0 | 0 |
| DYS- | A) PLACEBO | 51 | 0 | Treatment | 0.404 | 0 | 0 | 0 | 0 |
| MENORRHOEA | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 0 | 0 | 1 (100.0%) |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| PROSTATIC | A) PLACEBO | 51 | 0 | Treatment | 0.418 | 0 | 0 | 0 | 0 |
| DISORDER NOS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 1 (100.0%) | 0 |
| | F) MS 60 mg/NTX 0.1 mg | 48 | 0 | | | 0 | 0 | 0 | 0 |
| TESTICULAR | A) PLACEBO | 51 | 0 | Treatment | 0.418 | 0 | 0 | 0 | 0 |
| DISORDER NOS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |

RESPIRATORY, THORACIC AND MEDIASTINAL DISORDERS

| | | | | | | | | | |
|---|---|---|---|---|---|---|---|---|---|
| ALL EVENTS | A) PLACEBO | 51 | 0 | Treatment | 0.796 | 0 | 0 | 0 | 0 |
| | B) MS 60 mg | 53 | 2 (3.8%) | | | 2 | 2 (100.0%) | 0 | 0 |
| | C) NTX 0.01 mg | 51 | 2 (3.9%) | | | 2 | 1 (50.0%) | 0 | 1 (50.0%) |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 1 (2.0%) | | | 2 | 0 | 0 | 2 (100.0%) |
| | F) MS 60 mg/NTX 0.1 mg | 48 | 1 (2.1%) | | | 1 | 1 (100.0%) | 0 | 0 |
| COUGH | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 0 | 0 | 1 (100.0%) |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| EPISTAXIS | A) PLACEBO | 51 | 0 | Treatment | 0.542 | 0 | 0 | 0 | 0 |
| | B) MS 60 mg | 53 | 0 | | | 0 | 0 | 0 | 0 |
| | C) NTX 0.01 mg | 51 | 1 (2.0%) | | | 1 | 1 (100.0%) | 0 | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| NECK | A) PLACEBO | 51 | 0 | Treatment | 0.374 | 0 | 0 | 0 | 0 |
| TIGHTNESS | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 1 (100.0%) | 0 | 0 |
| RHINITIS NOS | A) PLACEBO | 51 | 0 | Treatment | 0.243 | 0 | 0 | 0 | 0 |
| | B) MS 60 mg | 53 | 2 (3.8%) | | | 2 | 2 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |
| SINUS | A) PLACEBO | 51 | 0 | Treatment | 0.418 | 0 | 0 | 0 | 0 |
| CONGESTION | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 0 | 0 | 1 (100.0%) |
| | F) MS 60 mg/NTX 0.1 mg | 48 | 0 | | | 0 | 0 | 0 | 0 |

SKIN & SUBCUTANEOUS TISSUE DISORDERS

| | | | | | | | | | |
|---|---|---|---|---|---|---|---|---|---|
| ALL EVENTS | A) PLACEBO | 51 | 0 | Treatment | 0.062 | 0 | 0 | 0 | 0 |
| | B) MS 60 mg | 53 | 4 (7.5%) | A-B | 0.045* | 6 | 5 (83.3%) | 1 (16.7%) | 0 |
| | C) NTX 0.01 mg | 51 | 1 (2.0%) | A-E | 0.006** | 1 | 0 | 1 (100.0%) | 0 |
| | D) MS 60 mg/NTX 0.001 mg | 50 | 3 (6.0%) | C-E | 0.027* | 5 | 2 (40.0%) | 3 (60.0%) | 0 |
| | E) MS 60 mg/NTX 0.01 mg | 51 | 7 (13.7%) | | | 8 | 4 (50.0%) | 3 (37.5%) | 1 (12.5%) |
| | F) MS 60 mg/NTX 0.1 mg | 48 | 3 (6.3%) | | | 4 | 0 | 2 (50.0%) | 2 (50.0%) |
| DERMATITIS | A) PLACEBO | 51 | 0 | Treatment | 0.567 | 0 | 0 | 0 | 0 |
| NOS | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| ECCHYMOSIS | A) PLACEBO | 51 | 0 | Treatment | 0.404 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 1 (2.0%) | | | 1 | 1 (100.0%) | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| ERYTHEMA | A) PLACEBO | 51 | 0 | Treatment | 0.446 | 0 | 0 | 0 | 0 |
| NEC | B) MS 60 mg | 53 | 1 (1.9%) | | | 1 | 1 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 0 | | | 0 | 0 | 0 | 0 |
| PHOTO- | A) PLACEBO | 51 | 0 | Treatment | 0.418 | 0 | 0 | 0 | 0 |
| SENSITIVITY | B) MS 60 mg | 53 | 0 | | | 0 | 0 | 0 | 0 |
| REACTION NOS | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| PRURITUS NOS | A) PLACEBO | 51 | 0 | Treatment | 0.056 | 0 | 0 | 0 | 0 |
| | B) MS 60 mg | 53 | 1 (1.9%) | A-E | 0.021* | 1 | 0 | 1 (100.0%) | 0 |
| | C) NTX 0.01 mg | 51 | 0 | C-E | 0.021* | 0 | 0 | 0 | 0 |
| | D) MS 60 mg/NTX 0.001 mg | 50 | 3 (6.0%) | | | 4 | 1 (25.0%) | 3 (75.0%) | 0 |
| | E) MS 60 mg/NTX 0.01 mg | 51 | 5 (9.8%) | | | 5 | 1 (20.0%) | 3 (60.0%) | 1 (20.0%) |
| | F) MS 60 mg/NTX 0.1 mg | 48 | 2 (4.2%) | | | 2 | 0 | 0 | 2 (100.0%) |
| SWEATING | A) PLACEBO | 51 | 0 | Treatment | 0.845 | 0 | 0 | 0 | 0 |
| INCREASED | B) MS 60 mg | 53 | 1 (1.9%) | | | 1 | 1 (100.0%) | 0 | 0 |
| | C) NTX 0.01 mg | 51 | 1 (2.0%) | | | 1 | 0 | 1 (100.0%) | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 1 (2.1%) | | | 1 | 0 | 1 (100.0%) | 0 |
| URTICARIA | A) PLACEBO | 51 | 0 | Treatment | 0.540 | 0 | 0 | 0 | 0 |
| NOS | B) MS 60 mg | 53 | 1 (1.9%) | | | 2 | 2 (100.0%) | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 1 (100.0%) | 0 |

VASCULAR DISORDERS

| | | | | | | | | | |
|---|---|---|---|---|---|---|---|---|---|
| ALL EVENTS | A) PLACEBO | 51 | 1 (2.0%) | Treatment | 0.153 | 1 | 0 | 1 (100.0%) | 0 |
| | B) MS 60 mg | 53 | 7 (13.2%) | A-B | 0.031* | 7 | 6 (85.7%) | 1 (14.3%) | 0 |
| | C) NTX 0.01 mg | 51 | 2 (3.9%) | A-F | 0.021* | 2 | 2 (100.0%) | 0 | 0 |
| | D) MS 60 mg/NTX 0.001 mg | 50 | 4 (8.0%) | | | 4 | 3 (75.0%) | 1 (25.0%) | 0 |
| | E) MS 60 mg/NTX 0.01 mg | 51 | 5 (9.8%) | | | 5 | 1 (20.0%) | 4 (80.0%) | 0 |
| | F) MS 60 mg/NTX 0.1 mg | 48 | 7 (14.6%) | | | 8 | 3 (37.5%) | 5 (62.5%) | 0 |
| FLUSHING | A) PLACEBO | 51 | 0 | Treatment | 0.418 | 0 | 0 | 0 | 0 |
| | B) MS 60 mg | 53 | 0 | | | 0 | 0 | 0 | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 1 (2.0%) | | | 1 | 1 (100.0%) | 0 | 0 |
| | F) MS 60 mg/NTX 0.1 mg | 48 | 0 | | | 0 | 0 | 0 | 0 |
| HOT FLUSHES | A) PLACEBO | 51 | 0 | Treatment | 0.540 | 0 | 0 | 0 | 0 |
| NOS | B) MS 60 mg | 53 | 1 (1.9%) | | | 1 | 0 | 1 (100.0%) | 0 |
| | C) NTX 0.01 mg | 51 | 0 | | | 0 | 0 | 0 | 0 |
| | D) MS 60 mg/NTX 0.001 mg | 50 | 0 | | | 0 | 0 | 0 | 0 |
| | E) MS 60 mg/NTX 0.01 mg | 51 | 0 | | | 0 | 0 | 0 | 0 |
| | F) MS 60 mg/NTX 0.1 mg | 48 | 1 (2.1%) | | | 1 | 0 | 1 (100.0%) | 0 |
| HYPER- | A) PLACEBO | 51 | 0 | Treatment | 0.500 | 0 | 0 | 0 | 0 |
| TENSION NOS | B) MS 60 mg | 53 | 1 (1.9%) | | | 1 | 1 (100.0%) | 0 | 0 |
| | C) NTX 0.01 mg | 51 | 1 (2.0%) | | | 1 | 1 (100.0%) | 0 | 0 |
| | D) MS 60 mg/NTX 0.001 mg | 50 | 3 (6.0%) | | | 3 | 2 (66.7%) | 1 (33.3%) | 0 |
| | E) MS 60 mg/NTX 0.01 mg | 51 | 1 (2.0%) | | | 1 | 0 | 1 (100.0%) | 0 |
| | F) MS 60 mg/NTX 0.1 mg | 48 | 1 (2.1%) | | | 1 | 1 (100.0%) | 0 | 0 |
| VASO- | A) PLACEBO | 51 | 1 (2.0%) | Treatment | 0.087 | 1 | 0 | 1 (100.0%) | 0 |
| DILATATION | B) MS 60 mg | 53 | 5 (9.4%) | A-F | 0.040* | 5 | 5 (100.0%) | 0 | 0 |
| | C) NTX 0.01 mg | 51 | 1 (2.0%) | C-F | 0.040* | 1 | 1 (100.0%) | 0 | 0 |
| | D) MS 60 mg/NTX 0.001 mg | 50 | 1 (2.0%) | D-F | 0.043* | 1 | 1 (100.0%) | 0 | 0 |
| | E) MS 60 mg/NTX 0.01 mg | 51 | 3 (5.9%) | | | 3 | 0 | 3 (100.0%) | 0 |
| | F) MS 60 mg/NTX 0.1 mg | 48 | 6 (12.5%) | | | 6 | 2 (33.3%) | 4 (66.7%) | 0 |

[1] P-VALUES ARE FROM CHISQ TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
*, **, ***P-VALUE <=0.05, <=0.01, or <=0.001 RESPECTIVELY.

TABLE 39B
SELECTED ADVERSE EVENTS
INTENT-TO-TREAT POPULATION, ALL PATIENTS

| | | Total | No. of | | | | | | |
|---|---|---|---|---|---|---|---|---|---|
| Body System | | No. of | Patients | | P-Value | No. of | | | |
| Adverse Events | Treatment | Patients | w/Event | Source | [1] | Events | Mild | Moderate | Severe |
| DIZZINESS | A) PLACEBO | 51 | 2 (3.9%) | Treatment | <.001*** | 2 | 0 | 2 (100.0%) | 0 |
| (EXC | B) MS 60 mg | 53 | 19 (35.8%) | A-B | <.001*** | 21 | 4 (19.0%) | 14 (66.7%) | 3 (14.3%) |
| VERTIGO) | C) NTX 0.01 mg | 51 | 2 (3.9%) | A-D | <.001*** | 2 | 2 (100.0%) | 0 | 0 |
| | D) MS 60 mg/NTX 0.001 mg | 50 | 18 (36.0%) | A-E | <.001*** | 19 | 7 (36.8%) | 11 (57.9%) | 1 (5.3%) |
| | E) MS 60 mg/NTX 0.01 mg | 51 | 20 (39.2%) | A-F | <.001*** | 23 | 10 (43.5%) | 12 (52.2%) | 1 (4.3%) |
| | F) MS 60 mg/NTX 0.1 mg | 48 | 16 (33.3%) | B-C | <.001*** | 19 | 7 (36.8%) | 9 (47.4%) | 3 (15.8) |
| | | | | C-D | <.001*** | | | | |
| | | | | C-E | <.001*** | | | | |
| | | | | C-F | <.001*** | | | | |
| NAUSEA | A) PLACEBO | 51 | 7 (13.7%) | Treatment | <.001*** | 8 | 3 (37.5%) | 2 (25.0%) | 3 (37.5%) |
| | B) MS 60 mg | 53 | 27 (50.9%) | A-B | <.001*** | 31 | 12 (38.7%) | 15 (48.4%) | 4 (12.9%) |
| | C) NTX 0.01 mg | 51 | 9 (17.6%) | A-D | <.001*** | 10 | 3 (30.0%) | 5 (50.0%) | 2 (20.0%) |
| | D) MS 60 mg/NTX 0.001 mg | 50 | 30 (60.0%) | A-E | <.001*** | 31 | 9 (29.0%) | 16 (51.6%) | 6 (19.4%) |
| | E) MS 60 mg/NTX 0.01 mg | 51 | 27 (52.9%) | A-F | <.001*** | 31 | 9 (29.0%) | 12 (38.7%) | 10 (32.3%) |
| | F) MS 60 mg/NTX 0.1 mg | 48 | 26 (54.2%) | B-C | <.001*** | 28 | 7 (25.0%) | 19 (67.9%) | 2 (7.1%) |
| | | | | C-D | <.001*** | | | | |
| | | | | C-E | <.001*** | | | | |
| | | | | C-F | <.001*** | | | | |
| SOMNOLENCE | A) PLACEBO | 51 | 0 | Treatment | <.001*** | 0 | 0 | 0 | 0 |
| | B) MS 60 mg | 53 | 11 (20.8%) | A-B | <.001*** | 13 | 2 (15.4%) | 9 (69.2%) | 2 (15.4%) |
| | C) NTX 0.01 mg | 51 | 0 | A-D | 0.005** | 0 | 0 | 0 | 0 |
| | D) MS 60 mg/NTX 0.001 mg | 50 | 7 (14.0%) | A-E | 0.003** | 8 | 4 (50.0%) | 4 (50.0%) | 0 |
| | E) MS 60 mg/NTX 0.01 mg | 51 | 8 (15.7%) | A-F | <.001*** | 8 | 4 (50.0%) | 4 (50.0%) | 0 |
| | F) MS 60 mg/NTX 0.1 mg | 48 | 12 (25.0%) | B-C | <.001*** | 12 | 6 (50.0%) | 5 (41.7%) | 1 (8.3%) |
| | | | | C-D | 0.005** | | | | |
| | | | | C-E | 0.003** | | | | |
| | | | | C-F | <.001*** | | | | |
| VOMITING NOS | A) PLACEBO | 51 | 4 (7.8%) | Treatment | <.001*** | 4 | 1 (25.0%) | 0 | 3 (75.0%) |
| | B) MS 60 mg | 53 | 25 (47.2%) | A-B | <.001*** | 26 | 4 (15.4%) | 7 (26.9%) | 15 (57.7%) |
| | C) NTX 0.01 mg | 51 | 7 (13.7%) | A-D | <.001*** | 7 | 1 (14.3%) | 1 (14.3%) | 5 (71.4%) |
| | D) MS 60 mg/NTX 0.001 mg | 50 | 27 (54.0%) | A-E | <.001*** | 29 | 3 (10.3%) | 9 (31.0%) | 17 (58.6%) |
| | E) MS 60 mg/NTX 0.01 mg | 51 | 25 (49.0%) | A-F | <.001*** | 29 | 4 (13.8%) | 5 (17.2%) | 20 (69.0%) |
| | F) MS 60 mg/NTX 0.1 mg | 48 | 27 (56.3%) | B-C | <.001*** | 33 | 3 (9.1%) | 6 (18.2%) | 24 (72.7%) |
| | | | | C-D | <.001*** | | | | |
| | | | | C-E | <.001*** | | | | |
| | | | | C-F | <.001*** | | | | |

[1] P-VALUES ARE FROM CHISQ TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
*, **, ***P-VALUE <=0.05, <=0.01, or <=0.001 RESPECTIVELY.

### EXAMPLE 4

The results from the clinical study using morphine alone and in combination with low doses of naltrexone as described in Example 3 were analyzed by gender.

The results for females and males from the Example 3 clinical study are shown in the following Tables and Figures.

A total of 304 subjects were randomized; among them 302 subjects were deemed evaluable. Tables 40A and 40B show the number of female and male subjects separately.

TABLE 40A
Analysis Populations, Female Patients
Treatments

| | | | | MS (60 mg) | MS (60 mg) | MS (60 mg) | |
|---|---|---|---|---|---|---|---|
| | | | NTX | NTX | NTX | NTX | |
| | Placebo | MS (60 mg) | 0.01 MG | (0.001 mg) | (0.01 mg) | (0.1 mg) | Total |
| Patients Enrolled [1] | 32 | 28 | 30 | 18 | 28 | 26 | 162 |
| Safety | 32 (100.0%) | 28 (100.0%) | 30 (100.0%) | 18 (100.0%) | 28 (100.0%) | 26 (100.0%) | 162 (100.0%) |
| Intent-To-Treat | 32 (100.0%) | 28 (100.0%) | 30 (100.0%) | 18 (100.0%) | 28 (100.0%) | 26 (100.0%) | 162 (100.0%) |
| Evaluable | 32 (100.0%) | 28 (100.0%) | 30 (100.0%) | 17 (94.4%) | 28 (100.0%) | 26 (100.0%) | 161 (99.4%) |

[1] PATIENTS WITH DEMOGRAPHIC INFORMATION.

TABLE 40B
Analysis Populations, Male Patients
Treatments

| | | | | MS (60 mg) | MS (60 mg) | MS (60 mg) | |
|---|---|---|---|---|---|---|---|
| | | | NTX | NTX | NTX | NTX | |
| | Placebo | MS (60 mg) | 0.01 MG | (0.001 mg) | (0.01 mg) | (0.1 mg) | Total |
| Patients Enrolled [1] | 19 | 25 | 21 | 32 | 23 | 22 | 142 |
| Safety | 19 (100.0%) | 25 (100.0%) | 21 (100.0%) | 32 (100.0%) | 23 (100.0%) | 22 (100.0%) | 142 (100.0%) |
| Intent-To-Treat | 19 (100.0%) | 25 (100.0%) | 21 (100.0%) | 32 (100.0%) | 23 (100.0%) | 22 (100.0%) | 142 (100.0%) |
| Evaluable | 19 (100.0%) | 25 (100.0%) | 21 (100.0%) | 32 (100.0%) | 23 (100.0%) | 21 (95.5%) | 141 (99.3%) |

[1] PATIENTS WITH DEMOGRAPHIC INFORMATION.

The demographic and baseline characteristics were summarized by treatment groups as shown in Table 41A for females and Table 41B for males.

The baseline pain intensity scores and visual analog scores are shown in Tables 42A and 42C for females and Tables 42B and 42D for males.

TABLE 41A
Baseline Characteristics
Intent-To-Treat Population, Female Patients

| | | | | | MS (60 mg) | MS (60 mg) | MS (60 mg) | | |
|---|---|---|---|---|---|---|---|---|---|
| | | | MS | NTX 0.01 | with NTX | with NTX | with NTX | | P-Value |
| | | Placebo | (60 mg) | mg | (0.001 mg) | (0.01 mg) | (0.1 mg) | TOTAL | [1] |
| Age (yrs) | N | 32 | 28 | 30 | 18 | 28 | 26 | 162 | 0.315 |
| | Mean | 23.2 | 23.8 | 22.1 | 21.4 | 22.2 | 24.2 | 22.9 | |
| | SD | 3.82 | 6.46 | 3.99 | 3.26 | 3.27 | 6.51 | 4.80 | |
| | Median | 23.0 | 23.0 | 21.0 | 21.0 | 22.0 | 22.0 | 22.0 | |
| | Range | 16-31 | 17-49 | 16-34 | 16-28 | 16-28 | 17-40 | 16-49 | |
| Race/Ethnic | Caucasian | 17 (53.1%) | 18 (64.3%) | 20 (66.7%) | 11 (61.1%) | 21 (75.0%) | 19 (73.1%) | 106 (65.4%) | 0.518 |
| Origin | Black | 6 (18.8%) | 4 (14.3%) | 5 (16.7%) | 3 (16.7%) | 3 (10.7%) | 3 (11.5%) | 24 (14.8%) | |
| (N, %) [2] | Asian | 2 (6.3%) | 1 (3.6%) | 0 (0.0%) | 0 (0.0%) | 0 (0.0%) | 2 (7.7%) | 5 (3.1%) | |
| | Hispanic | 7 (21.9%) | 5 (17.9%) | 5 (16.7%) | 4 (22.2%) | 4 (14.3%) | 2 (7.7%) | 27 (16.7%) | |
| | Total | 32 | 28 | 30 | 18 | 28 | 26 | 162 | |
| Height (cm) | N | 32 | 28 | 30 | 18 | 28 | 26 | 162 | 0.148 |
| | Mean | 164.7 | 165.7 | 164.3 | 161.0 | 164.7 | 165.8 | 164.6 | |
| | SD | 5.81 | 7.40 | 5.22 | 5.44 | 6.98 | 6.55 | 6.36 | |
| | Median | 164.0 | 165.1 | 163.5 | 162.6 | 165.6 | 165.1 | 165.1 | |
| | Range | 152.4-175.3 | 152.0-190.5 | 154.9-176.0 | 149.9-170.2 | 151.0-177.8 | 157.5-184.0 | 149.9-190.5 | |
| Weight (kg) | N | 32 | 28 | 30 | 18 | 28 | 26 | 162 | 0.115 |
| | Mean | 66.7 | 70.4 | 72.2 | 60.3 | 72.7 | 70.9 | 69.4 | |
| | SD | 17.92 | 15.06 | 19.47 | 11.98 | 13.58 | 16.16 | 16.42 | |
| | Median | 61.2 | 67.3 | 62.9 | 58.0 | 73.4 | 71.4 | 65.6 | |
| | Range | 44.5-115.7 | 45.4-112.7 | 45.9-115.5 | 44.9-97.1 | 52.7-98.4 | 48.6-117.0 | 44.5-117.0 | |
| Number of | 3 | 9 (28.1%) | 11 (39.3%) | 6 (20.0%) | 5 (27.8%) | 8 (28.6%) | 8 (30.8%) | 47 (29.0%) | 0.738 |
| Third Molars | 4 | 22 (68.8%) | 17 (60.7%) | 23 (76.7%) | 13 (72.2%) | 20 (71.4%) | 17 (65.4%) | 112 (69.1%) | |
| Extracted | 5 | 0 (0.0%) | 0 (0.0%) | 1 (3.3%) | 0 (0.0%) | 0 (0.0%) | 0 (0.0%) | 1 (0.6%) | |
| (N, %) [3] | 6 | 1 (3.1%) | 0 (0.0%) | 0 (0.0%) | 0 (0.0%) | 0 (0.0%) | 0 (0.0%) | 1 (0.6%) | |
| | 7 | 0 (0.0%) | 0 (0.0%) | 0 (0.0%) | 0 (0.0%) | 0 (0.0%) | 1 (3.8%) | 1 (0.6%) | |
| | TOTAL | 32 | 28 | 30 | 18 | 28 | 26 | 162 | |
| Time | N | 32 | 28 | 30 | 18 | 28 | 26 | 162 | 0.680 |
| Between End | Mean | 154.7 | 139.5 | 146.5 | 143.9 | 152.7 | 142.3 | 147.0 | |
| of Surgery | SD | 36.57 | 37.97 | 35.85 | 41.45 | 35.59 | 52.82 | 39.87 | |
| and Study | Median | 149.0 | 136.5 | 148.0 | 129.5 | 146.5 | 136.0 | 145.0 | |
| Medication | Range | 92.0-241.0 | 81.0-221.0 | 80.0-210.0 | 89.0-230.0 | 98.0-244.0 | 81.0-333.0 | 80.0-333.0 | |
| (Minutes) | | | | | | | | | |

[1] FOR AGE, HEIGHT, WEIGHT, AND TIME BETWEEN END OF SURGERY AND STUDY MEDICATION, P-VALUES ARE FROM TWO-WAY ANALYSIS OF VARIANCE WITH TREATMENT AND SITE AS FACTORS; FOR RACE/ETHNIC ORIGIN, AND NUMBER OF THIRD MOLARS EXTRACTED, P-VALUES ARE FROM COCHRAN-MANTEL-HAENZEL TEST ADJUSTING FOR SITE.
[2] BLACK, ASIAN, HISPANIC, AND OTHER ARE COMBINED INTO ONE CATEGORY TO DERIVE P-VALUE.
[3] 4 OR MORE THIRD MOLARS EXTRACTED AS ONE CATEGORY TO DERIVE P-VALUE.

TABLE 41B
Baseline Characteristics
Intent-To-Treat Population, Male Patients

| | | | | | | | | MS (60 mg) | | MS (60 mg) | | MS (60 mg) | | | | |
|---|---|---|---|---|---|---|---|---|---|---|---|---|---|---|---|---|
| | | | | MS | | NTX | | with NTX | | with NTX | | with NTX | | | | P-Value |
| | | Placebo | | (60 mg) | | 0.01 mg | | (0.001 mg) | | (0.01 mg) | | (0.1 mg) | | TOTAL | | [1] |
| Age (yrs) | N | 19 | | 25 | | 21 | | 32 | | 23 | | 22 | | 142 | | 0.019* |
| | Mean | 21.4 | | 23.1 | | 26.6 | | 23.1 | | 26.5 | | 23.9 | | 24.1 | | |
| | SD | 3.72 | | 5.20 | | 6.15 | | 4.82 | | 7.57 | | 5.89 | | 5.85 | | |
| | Median | 21.0 | | 22.0 | | 26.0 | | 22.0 | | 23.0 | | 21.5 | | 22.0 | | |
| | Range | 16-31 | | 16-36 | | 18-41 | | 16-38 | | 18-41 | | 18-39 | | 16-41 | | |
| Race/Ethnic | Caucasian | 14 | (73.7%) | 17 | (68.0%) | 14 | (66.7%) | 20 | (62.5%) | 16 | (69.6%) | 16 | (72.7%) | 97 | (68.3%) | 0.961 |
| Origin | Black | 2 | (10.5%) | 4 | (16.0%) | 2 | (9.5%) | 4 | (12.5%) | 5 | (21.7%) | 2 | (9.1%) | 19 | (13.4%) | |
| (N, %) [2] | Asian | 0 | (0.0%) | 1 | (4.0%) | 0 | (0.0%) | 0 | (0.0%) | 0 | (0.0%) | 0 | (0.0%) | 1 | (0.7%) | |
| | Hispanic | 2 | (10.5%) | 3 | (12.0%) | 4 | (19.0%) | 7 | (21.9%) | 1 | (4.3%) | 3 | (13.6%) | 20 | (14.1%) | |
| | Other | 1 | (5.3%) | 0 | (0.0%) | 1 | (4.8%) | 1 | (3.1%) | 1 | (4.3%) | 1 | (4.5%) | 5 | (3.5%) | |
| | Total | 19 | | 25 | | 21 | | 32 | | 23 | | 22 | | 142 | | |
| Height | N | 19 | | 25 | | 21 | | 32 | | 23 | | 22 | | 142 | | 0.486 |
| (cm) | Mean | 178.9 | | 178.4 | | 177.2 | | 175.3 | | 176.4 | | 176.8 | | 177.0 | | |
| | SD | 5.68 | | 7.88 | | 7.23 | | 7.92 | | 6.74 | | 8.17 | | 7.38 | | |
| | Median | 177.8 | | 177.8 | | 177.8 | | 175.2 | | 177.0 | | 176.5 | | 177.0 | | |
| | Range | 170.2-190.5 | | 162.6-195.6 | | 160.0-190.5 | | 162.6-198.1 | | 162.6-191.0 | | 160.0-190.5 | | 160.0-198.1 | | |
| Weight | N | 19 | | 25 | | 21 | | 32 | | 23 | | 22 | | 142 | | 0.581 |
| (kg) | Mean | 84.4 | | 80.8 | | 89.6 | | 80.7 | | 82.8 | | 83.6 | | 83.3 | | |
| | SD | 17.84 | | 11.42 | | 15.39 | | 22.42 | | 15.52 | | 22.09 | | 18.05 | | |
| | Median | 81.2 | | 77.6 | | 86.4 | | 77.0 | | 78.2 | | 82.1 | | 78.5 | | |
| | Range | 57.1-129.1 | | 61.4-111.8 | | 69.4-120.7 | | 56.7-147.7 | | 61.7-111.6 | | 56.2-157.8 | | 56.2-157.8 | | |
| Number of | 3 | 4 | (21.1%) | 7 | (28.0%) | 3 | (14.3%) | 5 | (15.6%) | 5 | (21.7%) | 8 | (36.4%) | 32 | (22.5%) | 0.415 |
| Third | 4 | 14 | (73.7%) | 18 | (72.0%) | 16 | (76.2%) | 26 | (81.3%) | 18 | (78.3%) | 14 | (63.6%) | 106 | (74.6%) | |
| Molars | 5 | 1 | (5.3%) | 0 | (0.0%) | 2 | (9.5%) | 1 | (3.1%) | 0 | (0.0%) | 0 | (0.0%) | 4 | (2.8%) | |
| Extracted | TOTAL | 19 | | 25 | | 21 | | 32 | | 23 | | 22 | | 142 | | |
| (N, %) [3] | | | | | | | | | | | | | | | | |

| | | | | | | | | | |
|---|---|---|---|---|---|---|---|---|---|
| Time | N | 19 | 25 | 21 | 32 | 23 | 22 | 142 | 0.045* |
| Between | Mean | 149.8 | 142.9 | 166.8 | 171.2 | 153.1 | 180.7 | 161.2 | |
| End of | SD | 45.40 | 39.40 | 52.50 | 46.26 | 31.93 | 58.88 | 47.31 | |
| Surgery | Median | 152.0 | 137.0 | 160.0 | 169.5 | 149.0 | 186.0 | 155.5 | |
| and Study | Range | 58.0-263.0 | 74.0-277.0 | 93.0-294.0 | 92.0-275.0 | 85.0-218.0 | 93.0-348.0 | 58.0-348.0 | |
| Medication | | | | | | | | | |
| (Minutes) | | | | | | | | | |

[1] FOR AGE, HEIGHT, WEIGHT, AND TIME BETWEEN END OF SURGERY AND STUDY MEDICATION, P-VALUES ARE FROM TWO-WAY ANALYSIS OF VARIANCE WITH TREATMENT AND SITE AS FACTORS; FOR RACE/ETHNIC ORIGIN, AND NUMBER OF THIRD MOLARS EXTRACTED, P-VALUES ARE FROM COCHRAN-MANTEL-HAENZEL TEST ADJUSTING FOR SITE.
[2] BLACK, ASIAN, HISPANIC, AND OTHER ARE COMBINED INTO ONE CATEGORY TO DERIVE P-VALUE.
[3] 4 OR MORE THIRD MOLARS EXTRACTED AS ONE CATEGORY TO DERIVE P-VALUE.

TABLE 42A
Baseline Pain Intensity Scores
Intent-To-Treat Population, Female Patients

| | | | P-VALUE FOR PAIRWISE COMPARISONS | | | | | P-Value |
|---|---|---|---|---|---|---|---|---|
| | | | | | MS 60 mg | MS 60 mg | MS 60 mg | for |
| PAIN INTENSITY | | | | NTX | NTX | NTX | NTX | Overall |
| TREATMENT | MODERATE | SEVERE | MS 60 mg | 0.01 mg | 0.001 mg | 0.01 mg | 0.1 mg | Treatment |
| Placebo | 15 (46.9%) | 17 (53.1%) | 0.834 | 0.311 | 0.846 | 0.811 | 0.816 | 0.950 |
| MS 60 mg | 14 (50.0%) | 14 (50.0%) | | 0.459 | 0.697 | 0.968 | 0.987 | |
| NTX 0.01 MG | 18 (60.0%) | 12 (40.0%) | | | 0.304 | 0.454 | 0.461 | |
| MS 60 mg/NTX 0.001 mg | 8 (44.4%) | 10 (55.6%) | | | | 0.691 | 0.706 | |
| MS 60 mg/NTX 0.01 mg | 14 (50.0%) | 14 (50.0%) | | | | | 1.000 | |
| MS 60 mg/NTX 0.1 mg | 13 (50.0%) | 13 (50.0%) | | | | | | |

NOTE:
P-VALUES ARE FROM COCHRAN-MANTEL-HAENZEL TEST ADJUSTING FOR SITE.

TABLE 42B
Baseline Pain Intensity Scores
Intent-To-Treat Population, Male Patients

| | | | P-VALUE FOR PAIRWISE COMPARISONS | | | | | P-Value |
|---|---|---|---|---|---|---|---|---|
| | | | | | MS 60 mg | MS 60 mg | MS 60 mg | for |
| PAIN INTENSITY | | | | NTX | NTX | NTX | NTX | Overall |
| TREATMENT | MODERATE | SEVERE | MS 60 mg | 0.01 mg | 0.001 mg | 0.01 mg | 0.1 mg | Treatment |
| Placebo | 10 (52.6%) | 9 (47.4%) | 0.737 | 0.206 | 0.871 | 0.781 | 0.876 | 0.891 |
| MS 60 mg | 12 (48.0%) | 13 (52.0%) | | 0.290 | 0.833 | 0.953 | 0.859 | |
| NTX 0.01 MG | 7 (33.3%) | 14 (66.7%) | | | 0.204 | 0.303 | 0.257 | |
| MS 60 mg/NTX 0.001 mg | 16 (50.0%) | 16 (50.0%) | | | | 0.888 | 0.997 | |
| MS 60 mg/NTX 0.01 mg | 11 (47.8%) | 12 (52.2%) | | | | | 0.896 | |
| MS 60 mg/NTX 0.1 mg | 11 (50.0%) | 11 (50.0%) | | | | | | |

NOTE:
P-VALUES ARE FROM COCHRAN-MANTEL-HAENZEL TEST ADJUSTING FOR SITE.

TABLE 42C
Baseline Visual Analog Scale (VAS) Scores
Intent-To-Treat Population, Female Patients

| | | | | | | | | | | P-VALUE FOR PAIRWISE COMPARISONS | | | | | |
|---|---|---|---|---|---|---|---|---|---|---|---|---|---|---|---|
| BASELINE VAS SCORE | | | | | | | | | | | | MS | | | P-Value |
| | Moderate | | | Severe | | | | | | | | 60 mg | MS 60 mg | MS 60 mg | for |
| | [1] | | | [1] | | | Total | | | | NTX | NTX | NTX | NTX | Overall |
| TREATMENT | N | Mean | (SD) | N | Mean | (SD) | N | Mean | (SD) | MS 60 mg | 0.01 mg | 0.001 mg | 0.01 mg | 0.1 mg | Treatment |
| Placebo | 15 | 66.8 | (13.33) | 17 | 82.1 | (10.40) | 32 | 74.9 | (13.99) | 0.847 | 0.744 | 0.948 | 0.170 | 0.332 | 0.471 |
| MS 60 mg | 14 | 73.1 | (7.03) | 14 | 77.7 | (10.26) | 28 | 75.4 | (8.95) | | 0.899 | 0.919 | 0.131 | 0.262 | |
| NTX 0.01 mg | 18 | 70.8 | (10.71) | 12 | 83.1 | (7.46) | 30 | 75.7 | (11.21) | | | 0.830 | 0.097 | 0.206 | |
| MS 60 mg/ | 8 | 67.8 | (8.65) | 10 | 80.8 | (7.50) | 18 | 75.0 | (10.25) | | | | 0.216 | 0.369 | |
| NTX 0.001 mg | | | | | | | | | | | | | | | |
| MS 60 mg/ | 14 | 63.6 | (8.74) | 14 | 78.1 | (7.07) | 28 | 70.9 | (10.77) | | | | | 0.715 | |
| NTX 0.01 mg | | | | | | | | | | | | | | | |
| MS 60 mg/ | 13 | 63.6 | (8.48) | 13 | 80.2 | (9.37) | 26 | 71.9 | (12.18) | | | | | | |
| NTX 0.1 mg | | | | | | | | | | | | | | | |

NOTE:
P-VALUES ARE FROM TWO-WAY ANALYSIS OF VARIANCE WITH TREATMENT AND SITE AS FACTORS.
[1] BASELINE PAIN INTENSITY ON THE CATEGORICAL SCALE.

TABLE 42D
Baseline Visual Analog Scale (VAS) Scores
Intent-To-Treat Population, Male Patients

| | | | | | | | | | | P-VALUE FOR PAIRWISE COMPARISONS | | | | | |
|---|---|---|---|---|---|---|---|---|---|---|---|---|---|---|---|
| BASELINE VAS SCORE | | | | | | | | | | | | MS | | | P-Value |
| | Moderate | | | Severe | | | | | | | | 60 mg | MS 60 mg | MS 60 mg | for |
| | [1] | | | [1] | | | Total | | | | NTX | NTX | NTX | NTX | Overall |
| TREATMENT | N | Mean | (SD) | N | Mean | (SD) | N | Mean | (SD) | MS 60 mg | 0.01 mg | 0.001 mg | 0.01 mg | 0.1 mg | Treatment |
| Placebo | 10 | 72.2 | (11.64) | 9 | 83.4 | (6.17) | 19 | 77.5 | (10.86) | 0.198 | 0.642 | 0.192 | 0.345 | 0.283 | 0.765 |
| MS 60 mg | 12 | 66.2 | (8.28) | 13 | 79.3 | (6.29) | 25 | 73.0 | (9.80) | | 0.407 | 0.957 | 0.729 | 0.847 | |
| NTX 0.01 mg | 7 | 67.1 | (8.38) | 14 | 79.9 | (7.06) | 21 | 75.6 | (9.55) | | | 0.410 | 0.629 | 0.534 | |
| MS 60 mg/ | 16 | 64.0 | (6.90) | 16 | 82.6 | (10.03) | 32 | 73.3 | (12.70) | | | | 0.754 | 0.880 | |
| NTX 0.001 mg | | | | | | | | | | | | | | | |
| MS 60 mg/ | 11 | 62.8 | (9.14) | 12 | 84.9 | (9.41) | 23 | 74.3 | (14.48) | | | | | 0.883 | |
| NTX 0.01 mg | | | | | | | | | | | | | | | |
| MS 60 mg/ | 11 | 66.3 | (7.16) | 11 | 81.3 | (5.29) | 22 | 73.8 | (9.83) | | | | | | |
| NTX 0.1 mg | | | | | | | | | | | | | | | |

NOTE:
P-VALUES ARE FROM TWO-WAY ANALYSIS OF VARIANCE WITH TREATMENT AND SITE AS FACTORS.
[1] BASELINE PAIN INTENSITY ON THE CATEGORICAL SCALE.

The TOTPAR results (4 hour, 6 hour, 8 hour) are summarized in Tables 43A for females and 43B for males. In females, all of the active treatment groups exhibited mean TOTPAR scores that were higher than the placebo group score, except for the 8 hour TOTPAR for NTX 0.01 mg alone which was comparable to placebo. The morphine alone group had the highest mean TOTPAR scores, followed by the 0.1 mg NTX and the 0.01 mg NTX combination groups. In males, the mean TOTPAR scores for the 0.001 mg NTX, 0.01 mg NTX, and 0.1 mg NTX combination groups were higher than the mean TOTPAR score for MS alone.

TABLE 43A
Total Pain Relief Scores
Intent-to-Treat Population, Female Patients

| TOTAL PAIN RELIEF SCORE | | | | | | | | P-VALUE |
|---|---|---|---|---|---|---|---|---|
| TREATMENT | N | MEAN | SD | MIN | MEDIAN | MAX | SOURCE | [1] |

TOTAL PAIN RELIEF SCORE (0-4 HOURS)

| | | | | | | | | |
|---|---|---|---|---|---|---|---|---|
| A) Placebo | 32 | 1.10 | 2.069 | 0.0 | 0.00 | 7.4 | TREATMENT | <0.001*** |
| B) MS 60 mg | 28 | 5.40 | 3.696 | 0.0 | 6.38 | 11.0 | SITE | 0.061 |
| C) NTX 0.01 mg | 30 | 1.43 | 2.439 | 0.0 | 0.00 | 10.4 | TREATMENT BY SITE | 0.390 |
| D) MS 60 mg/NTX 0.001 mg | 18 | 4.07 | 4.370 | 0.0 | 2.88 | 12.3 | A-B | <0.001*** |
| E) MS 60 mg/NTX 0.01 mg | 28 | 4.28 | 3.642 | 0.0 | 4.06 | 12.1 | A-C | 0.581 |
| F) MS 60 mg/NTX 0.1 mg | 26 | 4.12 | 2.901 | 0.0 | 3.38 | 9.5 | A-D | <0.001*** |
| | | | | | | | A-E | <0.001*** |
| | | | | | | | A-F | <0.001*** |
| | | | | | | | B-C | <0.001*** |
| | | | | | | | B-D | 0.454 |
| | | | | | | | B-E | 0.167 |
| | | | | | | | B-F | 0.120 |
| | | | | | | | C-D | 0.002** |
| | | | | | | | C-E | 0.002** |
| | | | | | | | C-F | 0.005** |
| | | | | | | | D-E | 0.652 |
| | | | | | | | D-F | 0.530 |
| | | | | | | | E-F | 0.830 |

TOTAL PAIN RELIEF SCORE (0-6 HOURS)

| | | | | | | | | |
|---|---|---|---|---|---|---|---|---|
| A) Placebo | 32 | 2.04 | 4.118 | 0.0 | 0.00 | 13.4 | TREATMENT | <0.001*** |
| B) MS 60 mg | 28 | 8.64 | 6.015 | 0.0 | 10.06 | 18.4 | SITE | 0.147 |
| C) NTX 0.01 mg | 30 | 2.17 | 3.836 | 0.0 | 0.00 | 16.4 | TREATMENT BY SITE | 0.407 |
| D) MS 60 mg/NTX 0.001 mg | 18 | 6.57 | 7.369 | 0.0 | 3.88 | 20.3 | A-B | <0.001*** |
| E) MS 60 mg/NTX 0.01 mg | 28 | 6.94 | 5.805 | 0.0 | 6.06 | 17.1 | A-C | 0.793 |
| F) MS 60 mg/NTX 0.1 mg | 26 | 6.79 | 5.144 | 0.0 | 5.38 | 15.9 | A-D | 0.001** |
| | | | | | | | A-E | <0.001*** |
| | | | | | | | A-F | 0.001** |
| | | | | | | | B-C | <0.001*** |
| | | | | | | | B-D | 0.513 |
| | | | | | | | B-E | 0.247 |
| | | | | | | | B-F | 0.175 |
| | | | | | | | C-D | 0.002** |
| | | | | | | | D-E | 0.727 |
| | | | | | | | D-F | 0.586 |
| | | | | | | | E-F | 0.813 |

TOTAL PAIN RELIEF SCORE (0-8 HOURS)

| | | | | | | | | |
|---|---|---|---|---|---|---|---|---|
| A) Placebo | 32 | 2.94 | 6.136 | 0.0 | 0.00 | 19.4 | TREATMENT | <0.001*** |
| B) MS 60 mg | 28 | 11.46 | 8.279 | 0.0 | 12.56 | 26.4 | SITE | 0.215 |
| C) NTX 0.01 mg | 30 | 2.90 | 5.255 | 0.0 | 0.00 | 22.4 | TREATMENT BY SITE | 0.427 |
| D) MS 60 mg/NTX 0.001 mg | 18 | 8.93 | 10.292 | 0.0 | 4.88 | 28.3 | A-B | <0.001*** |
| E) MS 60 mg/NTX 0.01 mg | 28 | 9.57 | 8.088 | 0.0 | 8.06 | 22.9 | A-C | 0.873 |
| F) MS 60 mg/NTX 0.1 mg | 26 | 9.41 | 7.295 | 0.0 | 7.38 | 23.9 | A-D | 0.002** |
| | | | | | | | A-E | <0.001*** |
| | | | | | | | A-F | 0.002** |
| | | | | | | | B-C | <0.001*** |
| | | | | | | | B-D | 0.585 |
| | | | | | | | B-E | 0.371 |
| | | | | | | | B-F | 0.257 |
| | | | | | | | C-D | 0.004** |
| | | | | | | | C-E | 0.002** |
| | | | | | | | C-F | 0.006** |
| | | | | | | | D-E | 0.819 |
| | | | | | | | D-F | 0.649 |
| | | | | | | | E-F | 0.788 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, or <=0.001 RESPECTIVELY

TABLE 43B
Total Pain Relief Scores
Intent-to-Treat Population, Male Patients

| TOTAL PAIN RELIEF SCORE | | | | | | | | P-VALUE |
|---|---|---|---|---|---|---|---|---|
| TREATMENT | N | MEAN | SD | MIN | MEDIAN | MAX | SOURCE | [1] |

TOTAL PAIN RELIEF SCORE (0-4 HOURS)

| | | | | | | | | |
|---|---|---|---|---|---|---|---|---|
| A) Placebo | 19 | 2.31 | 2.931 | 0.0 | 1.38 | 11.3 | TREATMENT | 0.009** |
| B) MS 60 mg | 25 | 2.17 | 2.505 | 0.0 | 0.88 | 7.5 | SITE | 0.408 |
| C) NTX 0.01 mg | 21 | 1.36 | 2.551 | 0.0 | 0.00 | 7.8 | TREATMENT BY SITE | 0.226 |
| D) MS 60 mg/NTX 0.001 mg | 32 | 3.12 | 3.658 | 0.0 | 2.56 | 12.5 | A-B | 0.800 |
| E) MS 60 mg/NTX 0.01 mg | 23 | 4.15 | 4.528 | 0.0 | 3.63 | 14.5 | A-C | 0.337 |
| F) MS 60 mg/NTX 0.1 mg | 22 | 5.41 | 4.727 | 0.0 | 5.88 | 14.5 | A-D | 0.631 |
| | | | | | | | A-E | 0.123 |
| | | | | | | | A-F | 0.021* |
| | | | | | | | B-C | 0.442 |
| | | | | | | | B-D | 0.418 |
| | | | | | | | B-E | 0.055 |
| | | | | | | | B-F | 0.006** |
| | | | | | | | C-D | 0.115 |
| | | | | | | | C-E | 0.010* |
| | | | | | | | C-F | <0.001*** |
| | | | | | | | D-E | 0.214 |
| | | | | | | | D-F | 0.035* |
| | | | | | | | E-F | 0.413 |

TOTAL PAIN RELIEF SCORE (0-6 HOURS)

| | | | | | | | | |
|---|---|---|---|---|---|---|---|---|
| A) Placebo | 19 | 4.05 | 5.205 | 0.0 | 1.38 | 19.3 | TREATMENT | 0.008** |
| B) MS 60 mg | 25 | 3.73 | 4.616 | 0.0 | 0.88 | 13.5 | SITE | 0.319 |
| C) NTX 0.01 mg | 21 | 2.10 | 4.078 | 0.0 | 0.00 | 11.8 | TREATMENT BY SITE | 0.223 |
| D) MS 60 mg/NTX 0.001 mg | 32 | 5.46 | 6.292 | 0.0 | 3.81 | 20.5 | A-B | 0.786 |
| E) MS 60 mg/NTX 0.01 mg | 23 | 6.89 | 7.329 | 0.0 | 5.88 | 22.5 | A-C | 0.261 |
| F) MS 60 mg/NTX 0.1 mg | 22 | 9.26 | 7.843 | 0.0 | 10.69 | 22.5 | A-D | 0.601 |
| | | | | | | | A-E | 0.168 |
| | | | | | | | A-F | 0.022* |
| | | | | | | | B-C | 0.354 |
| | | | | | | | B-D | 0.381 |
| | | | | | | | B-E | 0.078 |
| | | | | | | | B-F | 0.006** |
| | | | | | | | C-D | 0.072 |
| | | | | | | | C-E | 0.010* |
| | | | | | | | C-F | <0.001*** |
| | | | | | | | D-E | 0.312 |
| | | | | | | | D-F | 0.041* |
| | | | | | | | E-F | 0.328 |

TOTAL PAIN RELIEF SCORE (0-8 HOURS)

| | | | | | | | | |
|---|---|---|---|---|---|---|---|---|
| A) Placebo | 19 | 5.78 | 7.531 | 0.0 | 1.38 | 26.3 | TREATMENT | 0.007** |
| B) MS 60 mg | 25 | 5.31 | 6.793 | 0.0 | 0.88 | 19.5 | SITE | 0.275 |
| C) NTX 0.01 mg | 21 | 2.81 | 5.587 | 0.0 | 0.00 | 15.8 | TREATMENT BY SITE | 0.229 |
| D) MS 60 mg/NTX 0.001 mg | 32 | 7.77 | 9.088 | 0.0 | 4.38 | 28.5 | A-B | 0.795 |
| E) MS 60 mg/NTX 0.01 mg | 23 | 9.59 | 10.287 | 0.0 | 7.88 | 30.5 | A-C | 0.240 |
| F) MS 60 mg/NTX 0.1 mg | 22 | 13.30 | 11.230 | 0.0 | 14.69 | 30.5 | A-D | 0.607 |
| | | | | | | | A-E | 0.199 |
| | | | | | | | A-F | 0.020* |
| | | | | | | | B-C | 0.319 |
| | | | | | | | B-D | 0.393 |
| | | | | | | | B-E | 0.099 |
| | | | | | | | B-F | 0.005** |
| | | | | | | | C-D | 0.064 |
| | | | | | | | C-E | 0.011* |
| | | | | | | | C-F | <0.001*** |
| | | | | | | | D-E | 0.362 |
| | | | | | | | D-F | 0.036* |
| | | | | | | | E-F | 0.264 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***P-VALUE <=0.05, <=0.01, or <=0.001 RESPECTIVELY.

Tables 44A for females and 44B for males summarize the results of the 4, 6, and 8 hour SPID results and the 4 hour SPID results are shown in FIGS. 23B for females and 23C for males. In females, the NTX 0.01 mg alone and the placebo groups had the lowest mean SPID scores for 4, 6, and 8 hours. The MS alone and the 0.001 mg NTX combination groups had the highest mean SPID scores.

In males, the MS alone group had the lowest mean SPID scores. All of the combination groups had higher mean SPID scores than the MS alone, placebo, or NTX alone groups, and the 0.1 mg NTX combination group had the highest mean scores.

TABLE 44A
Sum of Pain Intensity Differences
Intent-To-Treat Population, Female Patients

| SUM OF PAIN INTENSITY DIFFERENCES [1] | | | | | | | | P-VALUE |
|---|---|---|---|---|---|---|---|---|
| | N | MEAN | SD | MIN | MEDIAN | MAX | SOURCE | [2] |

SUM OF PAIN INTENSITY DIFFERENCES (0-4 HOURS)

| | | | | | | | | |
|---|---|---|---|---|---|---|---|---|
| A) Placebo | 32 | −0.52 | 2.030 | −4 | 0.00 | 6 | TREATMENT | <0.001*** |
| B) MS 60 mg | 28 | 1.90 | 2.639 | −4 | 2.19 | 6 | SITE | 0.107 |
| C) NTX 0.01 mg | 30 | −1.02 | 2.275 | −4 | 0.00 | 4 | TREATMENT BY SITE | 0.308 |
| D) MS 60 mg/NTX 0.001 mg | 18 | 1.69 | 3.354 | −3 | 0.44 | 10 | A-B | <0.001*** |
| E) MS 60 mg/NTX 0.01 mg | 28 | 1.17 | 3.057 | −4 | 0.31 | 7 | A-C | 0.532 |
| F) MS 60 mg/NTX 0.1 mg | 26 | 1.16 | 2.331 | −3 | 0.13 | 6 | A-D | <0.001*** |
| | | | | | | | A-E | 0.020* |
| | | | | | | | A-F | 0.020* |
| | | | | | | | B-C | <0.001*** |
| | | | | | | | B-D | 0.820 |
| | | | | | | | B-E | 0.203 |
| | | | | | | | B-F | 0.238 |
| | | | | | | | C-D | <0.001*** |
| | | | | | | | C-E | 0.004** |
| | | | | | | | C-F | 0.004** |
| | | | | | | | D-E | 0.181 |
| | | | | | | | D-F | 0.208 |
| | | | | | | | E-F | 0.952 |

SUM OF PAIN INTENSITY DIFFERENCES (0-6 HOURS)

| | | | | | | | | |
|---|---|---|---|---|---|---|---|---|
| A) Placebo | 32 | −0.74 | 3.517 | −6 | 0.00 | 10 | TREATMENT | <0.001*** |
| B) MS 60 mg | 28 | 3.08 | 4.471 | −6 | 3.56 | 11 | SITE | 0.286 |
| C) NTX 0.01 mg | 30 | −1.57 | 3.534 | −6 | 0.00 | 6 | TREATMENT BY SITE | 0.355 |
| D) MS 60 mg/NTX 0.001 mg | 18 | 2.85 | 5.629 | −5 | 0.44 | 16 | A-B | <0.001*** |
| E) MS 60 mg/NTX 0.01 mg | 28 | 1.95 | 4.804 | −6 | 0.56 | 11 | A-C | 0.520 |
| F) MS 60 mg/NTX 0.1 | 26 | 2.02 | 3.882 | −5 | 0.31 | 9 | A-D | 0.001** |
| | | | | | | | A-E | 0.023* |
| | | | | | | | A-F | 0.024* |
| | | | | | | | B-C | <0.001*** |
| | | | | | | | B-D | 0.751 |
| | | | | | | | B-E | 0.260 |
| | | | | | | | B-F | 0.290 |
| | | | | | | | C-D | 0.001*** |
| | | | | | | | C-E | 0.005** |
| | | | | | | | C-F | 0.005** |
| | | | | | | | D-E | 0.192 |
| | | | | | | | D-F | 0.214 |
| | | | | | | | E-F | 0.968 |

SUM OF PAIN INTENSITY DIFFERENCES (0-8 HOURS)

| | | | | | | | | |
|---|---|---|---|---|---|---|---|---|
| A) Placebo | 32 | −1.01 | 4.916 | −8 | 0.00 | 12 | TREATMENT | <0.001*** |
| B) MS 60 mg | 28 | 3.92 | 6.218 | −8 | 3.94 | 15 | SITE | 0.489 |
| C) NTX 0.01 mg | 30 | −2.10 | 4.803 | −8 | 0.00 | 8 | TREATMENT BY SITE | 0.410 |
| D) MS 60 mg/NTX 0.001 mg | 18 | 3.85 | 7.787 | −7 | 0.44 | 22 | A-B | 0.001** |
| E) MS 60 mg/NTX 0.01 mg | 28 | 2.81 | 6.743 | −8 | 0.56 | 15 | A-C | 0.544 |
| F) MS 60 mg/NTX 0.1 mg | 26 | 2.81 | 5.399 | −7 | 0.38 | 11 | A-D | 0.001** |
| | | | | | | | A-E | 0.020* |
| | | | | | | | A-F | 0.027* |
| | | | | | | | B-C | <0.001*** |
| | | | | | | | B-D | 0.689 |
| | | | | | | | B-E | 0.408 |
| | | | | | | | B-F | 0.391 |
| | | | | | | | C-D | <0.001*** |
| | | | | | | | C-E | 0.004** |
| | | | | | | | C-F | 0.007** |
| | | | | | | | D-E | 0.260 |
| | | | | | | | D-F | 0.251 |
| | | | | | | | E-F | 0.957 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
[2] P-VALUES ARE FROM TWO-WAY ANALYSIS OF VARIANCE AND ITS CONTRASTS WITH TREATMENT, SITE, AND TREATMENT BY SITE INTERACTION AS FACTORS
*, **, ***P-VALUE <=0.05, <=0.01, or <=0.001 RESPECTIVELY

TABLE 44B
Sum of Pain Intensity Differences
Intent-To-Treat Population, Male Patients

| SUM OF PAIN INTENSITY DIFFERENCES [1] | | | | | | | | P-VALUE |
|---|---|---|---|---|---|---|---|---|
| | N | MEAN | SD | MIN | MEDIAN | MAX | SOURCE | [2] |

SUM OF PAIN INTENSITY DIFFERENCES (0-4 HOURS)

| | | | | | | | | |
|---|---|---|---|---|---|---|---|---|
| A) Placebo | 19 | 0.22 | 2.672 | −4 | 0.00 | 5 | TREATMENT | 0.045* |
| B) MS 60 mg | 25 | −0.37 | 2.153 | −4 | 0.00 | 4 | SITE | 0.020* |
| C) NTX 0.01 mg | 21 | 0.02 | 2.423 | −4 | 0.00 | 7 | TREATMENT BY SITE | 0.378 |
| D) MS 60 mg/NTX 0.001 mg | 32 | 0.46 | 3.176 | −4 | 0.00 | 9 | A-B | 0.443 |
| E) MS 60 mg/NTX 0.01 mg | 23 | 1.20 | 3.343 | −4 | 0.00 | 11 | A-C | 0.781 |
| F) MS 60 mg/NTX 0.1 mg | 22 | 2.51 | 3.700 | −4 | 2.56 | 11 | A-D | 0.986 |
| | | | | | | | A-E | 0.353 |
| | | | | | | | A-F | 0.037* |
| | | | | | | | B-C | 0.619 |
| | | | | | | | B-D | 0.373 |
| | | | | | | | B-E | 0.073 |
| | | | | | | | B-F | 0.002** |
| | | | | | | | C-D | 0.741 |
| | | | | | | | C-E | 0.212 |
| | | | | | | | C-F | 0.015 |
| | | | | | | | D-E | 0.302 |
| | | | | | | | D-F | 0.019* |
| | | | | | | | E-F | 0.220 |

SUM OF PAIN INTENSITY DIFFERENCES (0-6 HOURS)

| | | | | | | | | |
|---|---|---|---|---|---|---|---|---|
| A) Placebo | 19 | 0.69 | 4.602 | −6 | 0.00 | 9 | TREATMENT | 0.056 |
| B) MS 60 mg | 25 | −0.39 | 3.540 | −6 | 0.00 | 7 | SITE | 0.018* |
| C) NTX 0.01 mg | 21 | 0.02 | 3.827 | −6 | 0.00 | 11 | TREATMENT BY SITE | 0.329 |
| D) MS 60 mg/NTX 0.001 mg | 32 | 1.15 | 5.216 | −6 | 0.00 | 15 | A-B | N/D |
| E) MS 60 mg/NTX 0.01 mg | 23 | 2.14 | 5.455 | −6 | 0.00 | 17 | A-C | N/D |
| F) MS 60 mg/NTX 0.1 mg | 22 | 4.28 | 6.198 | −6 | 4.56 | 17 | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |

SUM OF PAIN INTENSITY DIFFERENCES (0-8 HOURS)

| | | | | | | | | |
|---|---|---|---|---|---|---|---|---|
| A) Placebo | 19 | 1.16 | 6.607 | −8 | 0.00 | 13 | TREATMENT | 0.056 |
| B) MS 60 mg | 25 | −0.43 | 4.963 | −8 | 0.00 | 11 | SITE | 0.016* |
| C) NTX 0.01 mg | 21 | 0.02 | 5.237 | −8 | 0.00 | 15 | TREATMENT BY SITE | 0.341 |
| D) MS 60 mg/NTX 0.001 mg | 32 | 1.73 | 7.203 | −8 | 0.00 | 21 | A-B | N/D |
| E) MS 60 mg/NTX 0.01 mg | 23 | 3.05 | 7.687 | −8 | 0.00 | 23 | A-C | N/D |
| F) MS 60 mg/NTX 0.1 mg | 22 | 6.10 | 8.757 | −8 | 6.56 | 23 | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
[2] P-VALUES ARE FROM TWO-WAY ANALYSIS OF VARIANCE AND ITS CONTRASTS WITH TREATMENT, SITE, AND TREATMENT BY SITE INTERACTION AS FACTORS
*, **, ***P-VALUE <=0.05, <=0.01, or <=0.001 RESPECTIVELY

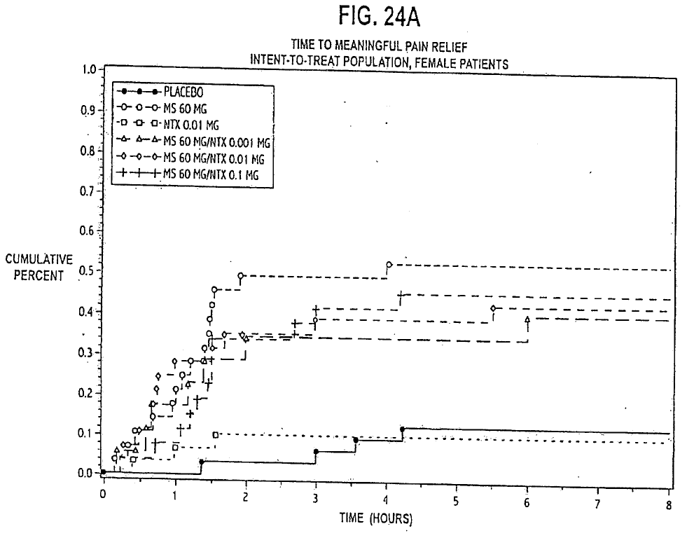

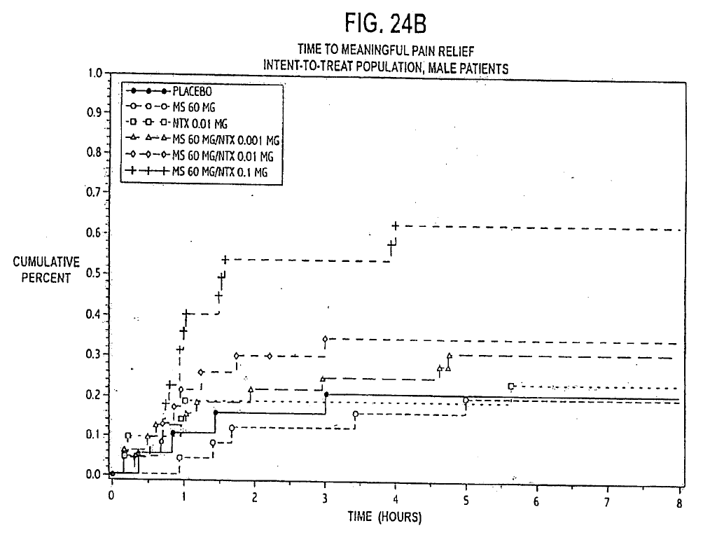

FIGS. 24A for females and 24B for males are visual presentations of the summary and analysis of time to onset of meaningful pain relief scores presented in Tables 45A for females and 45B for males. In females, the median time to onset of meaningful pain relief was shortest for the MS alone group and comparable for all other groups. In males, the 0.1 mg NTX combination group had the shortest median time to onset of meaningful pain relief while all other groups were comparable.

TABLE 45A
Time To Onset of Meaningful Pain Relief
Intent-To-Treat Population, Female Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 32 | >8:00 | (>8:00, >8:00) | TREATMENT | <0.001*** | <0.001*** |
| B) MS 60 mg | 28 | 2.57 | (1.28, >8:00) | A-B | <0.001*** | <0.001*** |
| C) NTX 0.01 mg | 30 | >8:00 | (>8:00, >8:00) | A-C | 0.883 | 0.901 |
| D) MS 60 mg/NTX 0.001 mg | 18 | >8:00 | (1:24, >8:00) | A-D | 0.057 | 0.031* |
| E) MS 60 mg/NTX 0.01 mg | 28 | >8:00 | (1:42, >8:00) | A-E | 0.009** | 0.003** |
| F) MS 60 mg/NTX 0.1 mg | 26 | >8:00 | (1:31, >8:00) | A-F | 0.012* | 0.008** |
| | | | | B-C | <0.001*** | <0.001*** |
| | | | | B-D | 0.276 | 0.369 |
| | | | | B-E | 0.412 | 0.590 |
| | | | | B-F | 0.345 | 0.356 |
| | | | | C-D | 0.046* | 0.027* |
| | | | | C-E | 0.007** | 0.003** |
| | | | | C-F | 0.009** | 0.007** |
| | | | | D-E | 0.725 | 0.681 |
| | | | | D-F | 0.800 | 0.920 |
| | | | | E-F | 0.909 | 0.719 |

*, **, ***P-VALUE <=0.05, <=0.01, or <=0.001 RESPECTIVELY.

TABLE 45B
Time To Onset of Meaningful Pain Relief
Intent-To-Treat Population, Male Patients

| | | MEDIAN | 95% CONFIDENCE | | | |
|---|---|---|---|---|---|---|
| | | TIME | INTERVAL | TEST OF SURVIVAL CURVES | | |
| TREATMENT | N | (hh:mm) | (hh:mm) | SOURCE | LOG-RANK | WILCOXON |
| A) Placebo | 19 | >8:00 | (>8:00, >8:00) | TREATMENT | 0.007** | 0.026* |
| B) MS 60 mg | 25 | >8:00 | (>8:00, >8:00) | A-B | 0.918 | 0.868 |
| C) NTX 0.01 mg | 21 | >8:00 | (>8:00, >8:00) | A-C | 0.826 | 0.776 |
| D) MS 60 mg/NTX 0.001 mg | 32 | >8:00 | (>8:00, >8:00) | A-D | 0.469 | 0.454 |
| E) MS 60 mg/NTX 0.01 mg | 23 | >8:00 | (3:00, >8:00) | A-E | 0.343 | 0.313 |
| F) MS 60 mg/NTX 0.1 mg | 22 | 1:33 | (0:57, >8:00) | A-F | 0.001** | 0.005** |
| | | | | B-C | 0.733 | 0.633 |
| | | | | B-D | 0.363 | 0.309 |
| | | | | B-E | 0.260 | 0.204 |
| | | | | B-F | <0.001*** | 0.001** |
| | | | | C-D | 0.623 | 0.662 |
| | | | | C-E | 0.463 | 0.473 |
| | | | | C-F | 0.001** | 0.012* |
| | | | | D-E | 0.757 | 0.724 |
| | | | | D-F | 0.003** | 0.018* |
| | | | | E-F | 0.014* | 0.064 |

*, **, ***P-VALUE <=0.05, <=0.01, or <=0.001 RESPECTIVELY.

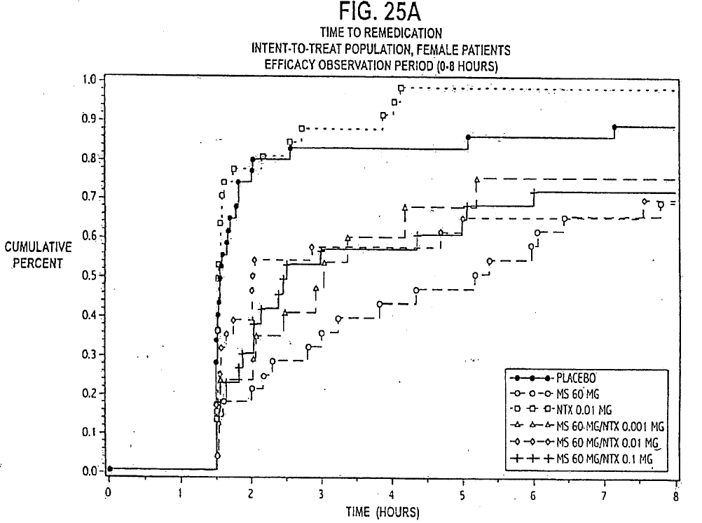

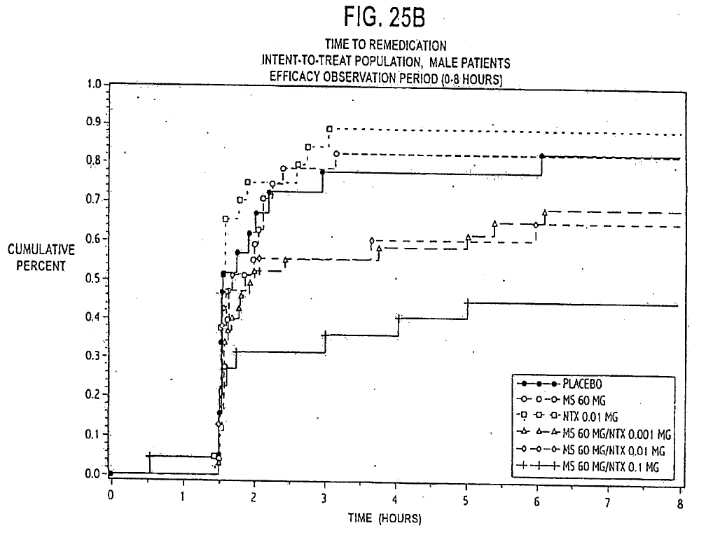

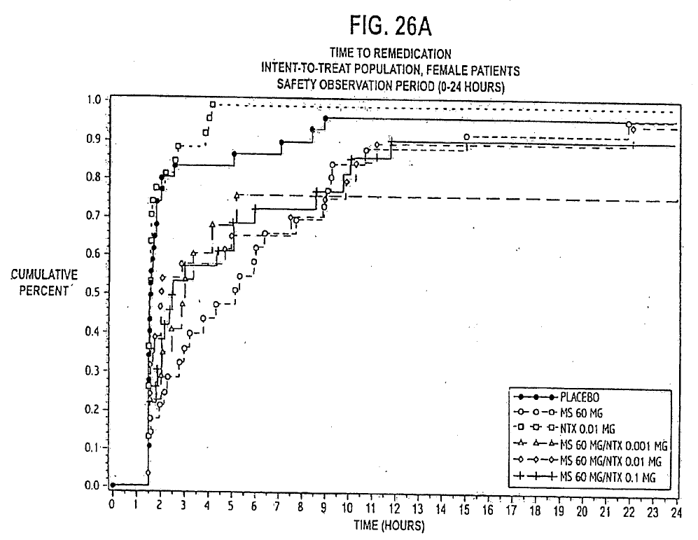

FIGS. 25A and 26A for females and 25B and 26B for males are visual presentations of the summary and analysis of time to remedication (rescue medication) up to 8 and 24 hours presented in Tables 46A for females and 46B for males. In females, the median time to remedication was longer for the NTX combination groups and the morphine alone group than the placebo and NTX alone groups. This was true at both 8 and 24 hours. In males, the median time to rescue medication was longest in the 0.1 mg NTX combination group and was similar for all other groups. This was true at both 8 and 24 hours.

TABLE 46A
Time To Rescue Medication
Intent-To-Treat Population, Female Patients

| | | | 95% | | | |
|---|---|---|---|---|---|---|
| | | | CONFIDENCE | | | |
| | | | INTERVAL | | | |
| | | MEDIAN | TIME | TEST OF SURVIVAL CURVES | | |
| TREATMENT | N | (hh:mm) | (hh:mm) | SOURCE | LOG-RANK | WILCOXON |

EFFICACY OBSERVATION PERIOD (0-8 HOURS)

| | | | | | | |
|---|---|---|---|---|---|---|
| A) Placebo | 32 | 1:34 | (1:31, 1:48) | TREATMENT | <0.001*** | <0.001*** |
| B) MS 60 mg | 28 | 5:11 | (3:01, 7:47) | A-B | <0.001*** | <0.001*** |
| C) NTX 0.01 mg | 30 | 1:33 | (1:32, 1:36) | A-C | 0.071 | 0.714 |
| D) MS 60 mg/NTX 0.001 mg | 18 | 3:03 | (2:03, 5:12) | A-D | 0.005** | 0.002** |
| E) MS 60 mg/NTX 0.01 mg | 28 | 2:03 | (1:40, 7:33) | A-E | 0.002** | 0.001** |
| F) MS 60 mg/NTX 0.1 mg | 26 | 2:29 | (2:03, 5:04) | A-F | 0.002** | <0.001*** |
| | | | | B-C | <0.001*** | <0.001*** |
| | | | | B-D | 0.566 | 0.339 |
| | | | | B-E | 0.459 | 0.136 |
| | | | | B-F | 0.495 | 0.309 |
| | | | | C-D | <0.001*** | <0.001*** |
| | | | | C-E | <0.001*** | <0.001*** |
| | | | | C-F | <0.001*** | <0.001*** |
| | | | | D-E | 0.943 | 0.728 |
| | | | | D-F | 0.984 | 0.938 |
| | | | | E-F | 0.953 | 0.623 |

EFFICACY OBSERVATION PERIOD (0-24HOURS)

| | | | | | | |
|---|---|---|---|---|---|---|
| A) Placebo | 32 | 1:34 | (1:31, 1:48) | TREATMENT | <0.001*** | <0.001*** |
| B) MS 60 mg | 28 | 5:11 | (3:01, 7:47) | A-B | <0.001*** | <0.001*** |
| C) NTX 0.01 mg | 30 | 1:33 | (1:32, 1:36) | A-C | 0.054 | 0.705 |
| D) MS 60 mg/NTX 0.001 mg | 18 | 3:03 | (2:03, 5:12) | A-D | <0.001*** | 0.001** |
| E) MS 60 mg/NTX 0.01 mg | 28 | 2:03 | (1:40, 7:33) | A-E | 0.002** | 0.001** |
| F) MS 60 mg/NTX 0.1 mg | 26 | 2:29 | (2:03, 5:04) | A-F | 0.002** | <0.001*** |
| | | | | B-C | <0.001*** | <0.001*** |
| | | | | B-D | 0.785 | 0.502 |
| | | | | B-E | 0.611 | 0.163 |
| | | | | B-F | 0.665 | 0.348 |
| | | | | C-D | <0.001*** | <0.001*** |
| | | | | C-E | <0.001*** | <0.001*** |
| | | | | C-F | <0.001*** | <0.001*** |
| | | | | D-E | 0.488 | 0.602 |
| | | | | D-F | 0.531 | 0.903 |
| | | | | E-F | 0.944 | 0.634 |

*, **, ***P-VALUE <=0.05, <=0.01, OR <=0.001 RESPECTIVELY

TABLE 46B
Time To Rescue Medication
Intent-To-Treat Population, Male Patients

| | | | 95% | | | |
|---|---|---|---|---|---|---|
| | | MEDIAN | CONFIDENCE | | | |
| | | TIME | INTERVAL | TEST OF SURVIVAL CURVES | | |
| TREATMENT | N | (hh:mm) | (hh:mm) | SOURCE | LOG-RANK | WILCOXON |

EFFICACY OBSERVATION PERIOD (0-8 HOURS)

| | | | | | | |
|---|---|---|---|---|---|---|
| A) Placebo | 19 | 1:34 | (1:32, 2:13) | TREATMENT | 0.027* | 0.029* |
| B) MS 60 mg | 25 | 1:53 | (1:36, 2:08) | A-B | 0.552 | 0.288 |
| C) NTX 0.01 mg | 21 | 1:34 | (1:32, 1:48) | A-C | 0.612 | 0.982 |
| D) MS 60 mg/NTX 0.001 mg | 32 | 1:59 | (1:35, 6:06) | A-D | 0.120 | 0.074 |
| E) MS 60 mg/NTX 0.01 mg | 23 | 1:42 | (1:31, >8:00) | A-E | 0.256 | 0.514 |
| F) MS 60 mg/NTX 0.1 mg | 22 | >8:00 | (1:45, >8:00) | A-F | 0.012* | 0.005** |
| | | | | B-C | 0.246 | 0.261 |
| | | | | B-D | 0.288 | 0.415 |
| | | | | B-E | 0.528 | 0.729 |
| | | | | B-F | 0.032* | 0.039* |
| | | | | C-D | 0.030* | 0.055 |
| | | | | C-E | 0.091 | 0.500 |
| | | | | C-F | 0.002** | 0.003** |
| | | | | D-E | 0.739 | 0.285 |
| | | | | D-F | 0.207 | 0.156 |
| | | | | E-F | 0.154 | 0.028* |

EFFICACY OBSERVATION PERIOD (0-24HOURS)

| | | | | | | |
|---|---|---|---|---|---|---|
| A) Placebo | 19 | 1:34 | (1:32, 2:13) | TREATMENT | 0.007** | 0.014* |
| B) MS 60 mg | 25 | 1:53 | (1:36, 2:08) | A-B | 0.517 | 0.272 |
| C) NTX 0.01 mg | 21 | 1:34 | (1:32, 1:48) | A-C | 0.298 | 0.984 |
| D) MS 60 mg/NTX 0.001 mg | 32 | 1:59 | (1:35, 6:06) | A-D | 0.253 | 0.086 |
| E) MS 60 mg/NTX 0.01 mg | 23 | 1:42 | (1:31, 9:35) | A-E | 0.255 | 0.491 |
| F) MS 60 mg/NTX 0.1 mg | 22 | 8:48 | (1:45, >24:00) | A-F | 0.008** | 0.002** |
| | | | | B-C | 0.078 | 0.223 |
| | | | | B-D | 0.603 | 0.502 |
| | | | | B-E | 0.575 | 0.727 |
| | | | | B-F | 0.027* | 0.021* |
| | | | | C-D | 0.021* | 0.056 |
| | | | | C-E | 0.027* | 0.448 |
| | | | | C-F | <0.001*** | <0.001*** |
| | | | | D-E | 0.919 | 0.338 |
| | | | | D-F | 0.055 | 0.067 |
| | | | | E-F | 0.106 | 0.014* |

*, **, ***P-VALUE <=0.05, <=0.01, or <=0.001 RESPECTIVELY.

Tables 47A for females and 47B for males present the summary and analysis of percent of subjects who took remedication (rescued) up to 8 and 24 hours. In females, the 0.001 mg NTX combination group had the lowest percentage of patients remedicating both at 8 and 24 hours. In males, at 8 hours, all three NTX combination groups had lower percentages of patients remedicating than the MS alone, NTX alone, or placebo groups. The 0.1 mg NTX combination group had the lowest percentage remedicating. At 24 hours, all groups were comparable except the MS and NTX 0.01 mg NTX and 0.1 mg NTX combination groups which had fewer patients remedicating.

TABLE 47A
Percent of Patients Rescued
Intent-To-Treat Population, Female Patients
RESCUED

| | | | | |
|---|---|---|---|---|
| TREATMENT | YES | NO | SOURCE | P-VALUE[1] |

EFFICACY OBSERVATION PERIOD (0-8 HOURS)

| | | | | |
|---|---|---|---|---|
| A) Placebo | 29 (90.6%) | 3 (9.4%) | TREATMENT | 0.013* |
| B) MS 60 mg | 19 (67.9%) | 9 (32.1%) | A-B | 0.029* |
| C) NTX 0.01 mg | 29 (96.7%) | 1 (3.3%) | A-C | 0.359 |
| D) MS 60 mg/NTX 0.001 mg | 12 (66.7%) | 6 (33.3%) | A-D | 0.039* |
| E) MS 60 mg/NTX 0.01 mg | 19 (67.9%) | 9 (32.1%) | A-E | 0.025* |
| F) MS 60 mg/NTX 0.1 mg | 19 (73.1%) | 7 (26.9%) | A-F | 0.079 |
| | | | B-C | 0.004** |
| | | | B-D | 0.924 |
| | | | B-E | 0.963 |
| | | | B-F | 0.700 |
| | | | C-D | 0.005** |
| | | | C-E | 0.003** |
| | | | C-F | 0.008** |
| | | | D-E | 0.975 |
| | | | D-F | 0.713 |
| | | | E-F | 0.565 |

EFFICACY OBSERVATION PERIOD (0-24 HOURS)

| | | | | |
|---|---|---|---|---|
| A) Placebo | 31 (96.9%) | 1 (3.1%) | TREATMENT | 0.015* |
| B) MS 60 mg | 26 (92.9%) | 2 (7.1%) | A-B | 0.447 |
| C) NTX 0.01 mg | 29 (96.7%) | 1 (3.3%) | A-C | 0.940 |
| D) MS 60 mg/NTX 0.001 mg | 12 (66.7%) | 6 (33.3%) | A-D | 0.004** |
| E) MS 60 mg/NTX 0.01 mg | 24 (85.7%) | 4 (14.3%) | A-E | 0.101 |
| F) MS 60 mg/NTX 0.1 mg | 23 (88.5%) | 3 (11.5%) | A-F | 0.218 |
| | | | B-C | 0.541 |
| | | | B-D | 0.022* |
| | | | B-E | 0.381 |
| | | | B-F | 0.587 |
| | | | C-D | 0.005** |
| | | | C-E | 0.118 |
| | | | C-F | 0.230 |
| | | | D-E | 0.163 |
| | | | D-F | 0.090 |
| | | | E-F | 0.673 |

[1] P-VALUES ARE FROM COCHRAN-MANTEL-HAENZEL TEST ADJUSTING FOR SITE.

TABLE 47B
Percent of Patients Rescued
Intent-To-Treat Population, Male Patients
RESCUED

| | | | | |
|---|---|---|---|---|
| TREATMENT | YES | NO | SOURCE | P-VALUE [1] |

EFFICACY OBSERVATION PERIOD (0-8 HOURS)

| | | | | |
|---|---|---|---|---|
| A) Placebo | 16 (84.2%) | 3 (15.8%) | TREATMENT | 0.010* |
| B) MS 60 mg | 21 (84.0%) | 4 (16.0%) | A-B | 0.997 |
| C) NTX 0.01 mg | 19 (90.5%) | 2 (9.5%) | A-C | 0.567 |
| D) MS 60 mg/NTX 0.001 mg | 22 (68.8%) | 10 (31.3%) | A-D | 0.230 |
| E) MS 60 mg/NTX 0.01 mg | 15 (65.2%) | 8 (34.8%) | A-E | 0.177 |
| F) MS 60 mg/NTX 0.1 mg | 10 (45.5%) | 12 (54.5%) | A-F | 0.008** |
| | | | B-C | 0.494 |
| | | | B-D | 0.191 |
| | | | B-E | 0.141 |
| | | | B-F | 0.006** |
| | | | C-D | 0.075 |
| | | | C-E | 0.057 |
| | | | C-F | 0.001** |
| | | | D-E | 0.798 |
| | | | D-F | 0.076 |
| | | | E-F | 0.147 |

EFFICACY OBSERVATION PERIOD (0-24 HOURS)

| | | | | |
|---|---|---|---|---|
| A) Placebo | 18 (94.7%) | 1 (5.3%) | TREATMENT | 0.003** |
| B) MS 60 mg | 23 (92.0%) | 2 (8.0%) | A-B | 0.722 |
| C) NTX 0.01 mg | 21 (100.0%) | 0 (0.0%) | A-C | 0.317 |
| D) MS 60 mg/NTX 0.001 mg | 30 (93.8%) | 2 (6.3%) | A-D | 0.890 |
| E) MS 60 mg/NTX 0.01 mg | 19 (82.6%) | 4 (17.4%) | A-E | 0.243 |
| F) MS 60 mg/NTX 0.1 mg | 14 (63.6%) | (8 (36.4%) | A-F | 0.014* |
| | | | B-C | 0.193 |
| | | | B-D | 0.809 |
| | | | B-E | 0.345 |
| | | | B-F | 0.019* |
| | | | C-D | 0.246 |
| | | | C-E | 0.055 |
| | | | C-F | 0.002** |
| | | | D-E | 0.200** |
| | | | D-F | 0.004** |
| | | | E-F | 0.131 |

[1] P-VALUES ARE FROM COCHRAN-MANTEL-HAENZEL TEST ADJUSTING FOR SITE.

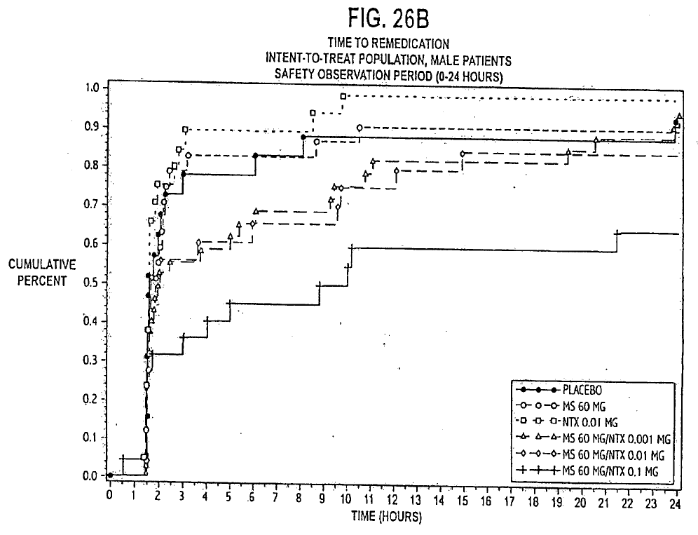

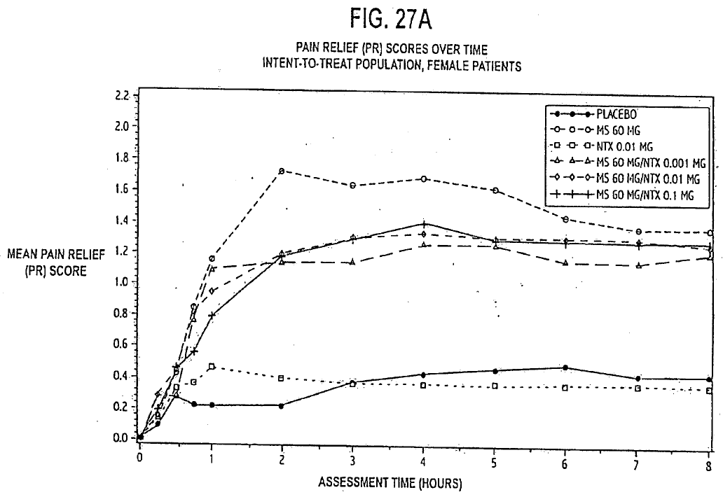

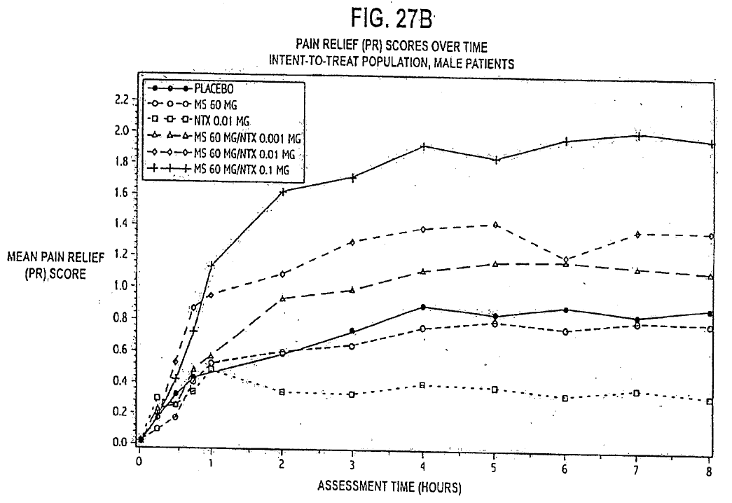

FIGS. 27A for females and 27B for males are visual presentations of the mean pain relief scores presented in Tables 48A for females and 48B for males. In females, from 45 minutes to 8 hours all three NTX combination groups, as well as the MS alone group, have higher mean pain relief scores than the placebo group. In males, the pain relief score of the MS alone group is not statistically different from the placebo group. All three NTX combination groups have higher mean pain relief scores than the placebo or morphine groups from 15 minutes to 8 hours. The 0.01 mg NTX and the 0.1 mg NTX combination groups have the highest pain relief scores.

TABLE 48A
Pain Relief (PR) Scores
Intent-To-Treat Population, Female Patients

| | PAIN RELIEF SCORE (PR) | | | | |
|---|---|---|---|---|---|
| TREATMENT | N | MEAN | SD | SOURCE | P-VALUE [1] |
| 15 MINUTES | | | | | |
| A) Placebo | 32 | 0.09 | 0.390 | Treatment | 0.778 |
| B) MS 60 mg | 28 | 0.14 | 0.448 | Site | 0.127 |
| C) NTX 0.01 mg | 30 | 0.13 | 0.434 | Treatment by Site | 0.275 |
| D) MS 60 mg/NTX 0.001 mg | 18 | 0.28 | 0.575 | A-B | N/D |
| E) MS 60 mg/NTX 0.01 mg | 28 | 0.29 | 0.713 | A-C | N/D |
| F) MS 60 mg/NTX 0.1 mg | 26 | 0.19 | 0.567 | A-D | N/D |
| | | | | A-E | N/D |
| | | | | A-F | N/D |
| | | | | B-C | N/D |
| | | | | B-D | N/D |
| | | | | B-E | N/D |
| | | | | B-F | N/D |
| | | | | C-D | N/D |
| | | | | C-E | N/D |
| | | | | C-F | N/D |
| | | | | D-E | N/D |
| | | | | D-F | N/D |
| | | | | E-F | N/D |
| 30 MINUTES | | | | | |
| A) Placebo | 32 | 0.28 | 0.581 | Treatment | 0.883 |
| B) MS 60 mg | 28 | 0.46 | 0.693 | Site | 0.205 |
| C) NTX 0.01 mg | 30 | 0.33 | 0.661 | Treatment by Site | 0.621 |
| D) MS 60 mg/NTX 0.001 mg | 18 | 0.28 | 0.461 | A-B | N/D |
| E) MS 60 mg/NTX 0.01 mg | 28 | 0.43 | 0.879 | A-C | N/D |
| F) MS 60 mg/NTX 0.1 mg | 26 | 0.46 | 0.811 | A-D | N/D |
| | | | | A-E | N/D |
| | | | | A-F | N/D |
| | | | | B-C | N/D |
| | | | | B-D | N/D |
| | | | | B-E | N/D |
| | | | | B-F | N/D |
| | | | | C-D | N/D |
| | | | | C-E | N/D |
| | | | | C-F | N/D |
| | | | | D-E | N/D |
| | | | | D-F | N/D |
| | | | | E-F | N/D |
| 45 MINUTES | | | | | |
| A) Placebo | 32 | 0.22 | 0.491 | Treatment | 0.015* |
| B) MS 60 mg | 28 | 0.86 | 0.848 | Site | 0.087 |
| C) NTX 0.01 mg | 30 | 0.37 | 0.669 | Treatment by Site | 0.390 |
| D) MS 60 mg/NTX 0.001 mg | 18 | 0.78 | 0.878 | A-B | 0.004** |
| E) MS 60 mg/NTX 0.01 mg | 28 | 0.82 | 1.020 | A-C | 0.521 |
| F) MS 60 mg/NTX 0.1 mg | 26 | 0.58 | 0.703 | A-D | 0.011* |
| | | | | A-E | 0.009** |
| | | | | A-F | 0.113 |
| | | | | B-C | 0.029* |
| | | | | B-D | 0.972 |
| | | | | B-E | 0.760 |
| | | | | B-F | 0.220 |
| | | | | C-D | 0.052 |
| | | | | C-E | 0.056 |
| | | | | C-F | 0.353 |
| | | | | D-E | 0.763 |
| | | | | D-F | 0.267 |
| | | | | E-F | 0.345 |
| 1 HOUR | | | | | |
| A) Placebo | 32 | 0.22 | 0.608 | Treatment | <0.001*** |
| B) MS 60 mg | 28 | 1.18 | 1.056 | Site | 0.019* |
| C) NTX 0.01 mg | 30 | 0.47 | 0.776 | Treatment by Site | 0.675 |
| D) MS 60 mg/NTX 0.001 mg | 18 | 1.11 | 1.132 | A-B | <0.001*** |
| E) MS 60 mg/NTX 0.01 mg | 28 | 0.96 | 0.962 | A-C | 0.285 |
| F) MS 60 mg/NTX 0.1 mg | 26 | 0.81 | 0.634 | A-D | <0.001*** |
| | | | | A-E | 0.002** |
| | | | | A-F | 0.012* |
| | | | | B-C | 0.002** |
| | | | | B-D | 0.935 |
| | | | | B-E | 0.253 |
| | | | | B-F | 0.113 |
| | | | | C-D | 0.006** |
| | | | | C-E | 0.050 |
| | | | | C-F | 0.153 |
| | | | | D-E | 0.280 |
| | | | | D-F | 0.141 |
| | | | | E-F | 0.630 |
| 1.5 HOURS | | | | | |
| A) Placebo | 32 | 0.22 | 0.491 | Treatment | <0.001*** |
| B) MS 60 mg | 28 | 1.54 | 1.036 | Site | 0.134 |
| C) NTX 0.01 mg | 30 | 0.40 | 0.724 | Treatment by Site | 0.217 |
| D) MS 60 mg/NTX 0.001 mg | 18 | 1.28 | 1.274 | A-B | <0.001*** |
| E) MS 60 mg/NTX 0.01 mg | 28 | 1.25 | 1.041 | A-C | 0.355 |
| F) MS 60 mg/NTX 0.1 mg | 26 | 1.19 | 0.801 | A-D | <0.001*** |
| | | | | A-E | <0.001*** |
| | | | | A-F | <0.001*** |
| | | | | B-C | <0.001*** |
| | | | | B-D | 0.687 |
| | | | | B-E | 0.173 |
| | | | | B-F | 0.098 |
| | | | | C-D | <0.001*** |
| | | | | C-E | 0.001** |
| | | | | C-F | 0.004** |
| | | | | D-E | 0.434 |
| | | | | D-F | 0.290 |
| | | | | E-F | 0.735 |
| 2 HOURS | | | | | |
| A) Placebo | 32 | 0.22 | 0.491 | Treatment | <0.001*** |
| B) MS 60 mg | 28 | 1.75 | 1.175 | Site | 0.042* |
| C) NTX 0.01 mg | 30 | 0.40 | 0.724 | Treatment by Site | 0.136 |
| D) MS 60 mg/NTX 0.001 mg | 18 | 1.17 | 1.425 | A-B | <0.001*** |
| E) MS 60 mg/NTX 0.01 mg | 28 | 1.21 | 1.067 | A-C | 0.368 |
| F) MS 60 mg/NTX 0.1 mg | 26 | 1.19 | 0.981 | A-D | <0.001*** |
| | | | | A-E | <0.001*** |
| | | | | A-F | <0.001*** |
| | | | | B-C | <0.001*** |
| | | | | B-D | 0.233 |
| | | | | B-E | 0.034* |
| | | | | B-F | 0.026* |
| | | | | C-D | 0.001** |
| | | | | C-E | 0.003** |
| | | | | C-F | 0.007** |
| | | | | D-E | 0.514 |
| | | | | D-F | 0.435 |
| | | | | E-F | 0.870 |
| 3 HOURS | | | | | |
| A) Placebo | 32 | 0.38 | 0.833 | Treatment | <0.001*** |
| B) MS 60 mg | 28 | 1.66 | 1.261 | Site | 0.125 |
| C) NTX 0.01 mg | 30 | 0.37 | 0.718 | Treatment by Site | 0.432 |
| D) MS 60 mg/NTX 0.001 mg | 18 | 1.17 | 1.425 | A-B | <0.001*** |
| E) MS 60 mg/NTX 0.01 mg | 28 | 1.32 | 1.188 | A-C | 0.866 |
| F) MS 60 mg/NTX 0.1 mg | 26 | 1.31 | 1.158 | A-D | 0.003** |
| | | | | A-E | 0.001** |
| | | | | A-F | 0.002** |
| | | | | B-C | <0.001*** |
| | | | | B-D | 0.399 |
| | | | | B-E | 0.264 |
| | | | | B-F | 0.217 |
| | | | | C-D | 0.006** |
| | | | | C-E | 0.002** |
| | | | | C-F | 0.005** |
| | | | | D-E | 0.903 |
| | | | | D-F | 0.802 |
| | | | | E-F | 0.879 |
| 4 HOURS | | | | | |
| A) Placebo | 32 | 0.44 | 0.982 | Treatment | <0.001*** |
| B) MS 60 mg | 28 | 1.71 | 1.301 | Site | 0.306 |
| C) NTX 0.01 mg | 30 | 0.37 | 0.718 | Treatment by Site | 0.529 |
| D) MS 60 mg/NTX 0.001 mg | 18 | 1.28 | 1.565 | A-B | <0.001*** |
| E) MS 60 mg/NTX 0.01 mg | 28 | 1.36 | 1.224 | A-C | 0.957 |
| F) MS 60 mg/NTX 0.1 mg | 26 | 1.42 | 1.238 | A-D | 0.005** |
| | | | | A-E | 0.003** |
| | | | | A-F | 0.003** |
| | | | | B-C | <0.001*** |
| | | | | B-D | 0.497 |
| | | | | B-E | 0.281 |
| | | | | B-F | 0.318 |
| | | | | C-D | 0.005** |
| | | | | C-E | 0.003** |
| | | | | C-F | 0.003** |
| | | | | D-E | 0.798 |
| | | | | D-F | 0.837 |
| | | | | E-F | 0.959 |
| 5 HOURS | | | | | |
| A) Placebo | 32 | 0.47 | 1.047 | Treatment | <0.001*** |
| B) MS 60 mg | 28 | 1.64 | 1.311 | Site | 0.463 |
| C) NTX 0.01 mg | 30 | 0.37 | 0.718 | Treatment by Site | 0.254 |
| D) MS 60 mg/NTX 0.001 mg | 18 | 1.28 | 1.565 | A-B | <0.001*** |
| E) MS 60 mg/NTX 0.01 mg | 28 | 1.32 | 1.188 | A-C | 0.889 |
| F) MS 60 mg/NTX 0.1 mg | 26 | 1.31 | 1.192 | A-D | 0.006** |
| | | | | A-E | 0.004** |
| | | | | A-F | 0.015* |
| | | | | B-C | <0.001*** |
| | | | | B-D | 0.679 |
| | | | | B-E | 0.401 |
| | | | | B-F | 0.246 |
| | | | | C-D | 0.005** |
| | | | | C-E | 0.004** |
| | | | | C-F | 0.013* |
| | | | | D-E | 0.753 |
| | | | | D-F | 0.542 |
| | | | | E-F | 0.727 |
| 6 HOURS | | | | | |
| A) Placebo | 32 | 0.50 | 1.107 | Treatment | 0.001** |
| B) MS 60 mg | 28 | 1.46 | 1.232 | Site | 0.535 |
| C) NTX 0.01 mg | 30 | 0.37 | 0.718 | Treatment by Site | 0.456 |
| D) MS 60 mg/NTX 0.001 mg | 18 | 1.17 | 1.505 | A-B | 0.002** |
| E) MS 60 mg/NTX 0.01 mg | 28 | 1.32 | 1.219 | A-C | 0.790 |
| F) MS 60 mg/NTX 0.1 mg | 26 | 1.31 | 1.158 | A-D | 0.028* |
| | | | | A-E | 0.006** |
| | | | | A-F | 0.021* |
| | | | | B-C | 0.001** |
| | | | | B-D | 0.666 |
| | | | | B-E | 0.737 |
| | | | | B-F | 0.502 |
| | | | | C-D | 0.018* |
| | | | | C-E | 0.003** |
| | | | | C-F | 0.013* |
| | | | | D-E | 0.886 |
| | | | | D-F | 0.870 |
| | | | | E-F | 0.725 |
| 7 HOURS | | | | | |
| A) Placebo | 32 | 0.44 | 1.014 | Treatment | <0.001*** |
| B) MS 60 mg | 28 | 1.39 | 1.227 | Site | 0.551 |
| C) NTX 0.01 mg | 30 | 0.37 | 0.718 | Treatment by Site | 0.427 |
| D) MS 60 mg/NTX 0.001 mg | 18 | 1.17 | 1.505 | A-B | 0.001** |
| E) MS 60 mg/NTX 0.01 mg | 28 | 1.32 | 1.219 | A-C | 0.988 |
| F) MS 60 mg/NTX 0.1 mg | 26 | 1.31 | 1.123 | A-D | 0.014* |
| | | | | A-E | 0.002** |
| | | | | A-F | 0.009** |
| | | | | B-C | 0.002** |
| | | | | B-D | 0.775 |
| | | | | B-E | 0.870 |
| | | | | B-F | 0.608 |
| | | | | C-D | 0.016* |
| | | | | C-E | 0.003** |
| | | | | C-F | 0.011* |
| | | | | D-E | 0.883 |
| | | | | D-F | 0.867 |
| | | | | E-F | 0.720 |
| 8 HOURS | | | | | |
| A) Placebo | 32 | 0.44 | 0.982 | Treatment | <0.001*** |
| B) MS 60 mg | 28 | 1.39 | 1.227 | Site | 0.364 |
| C) NTX 0.01 mg | 30 | 0.37 | 0.718 | Treatment by Site | 0.353 |
| D) MS 60 mg/NTX 0.001 mg | 18 | 1.22 | 1.592 | A-B | 0.002** |
| E) MS 60 mg/NTX 0.01 mg | 28 | 1.29 | 1.243 | A-C | 0.956 |
| F) MS 60 mg/NTX 0.1 mg | 26 | 1.31 | 1.123 | A-D | 0.008** |
| | | | | A-E | 0.004** |
| | | | | A-F | 0.011* |
| | | | | B-C | 0.002** |
| | | | | B-D | 0.957 |
| | | | | B-E | 0.793 |
| | | | | B-F | 0.611 |
| | | | | C-D | 0.009** |
| | | | | C-E | 0.004** |
| | | | | C-F | 0.012* |
| | | | | D-E | 0.861 |
| | | | | D-F | 0.694 |
| | | | | E-F | 0.797 |

[1] P-Values are from two-way analysis of variance and its contrasts with treatment, site, and treatment by site interaction as factors.
*, **, ***P-Value <=0.05, <=0.01, or <=0.001 respectively.
N/D: Not done (because overall P-Value not significant).

TABLE 48B
Pain Relief (PR) Scores
Intent-To-Treat Population, Male Patients

| | PAIN RELIEF SCORE (PR) | | | | | | | |
|---|---|---|---|---|---|---|---|---|
| TREATMENT | N | MEAN | SD | MIN | MEDIAN | MAX | SOURCE | P-VALUE [1] |
| 15 MINUTES | | | | | | | | |
| A) Placebo | 19 | 0.16 | 0.375 | | | | Treatment | 0.742 |
| B) MS 60 mg | 25 | 0.08 | 0.277 | | | | Site | 0.144 |
| C) NTX 0.01 mg | 21 | 0.29 | 0.644 | | | | Treatment by Site | 0.116 |
| D) MS 60 mg/NTX 0.001 mg | 32 | 0.22 | 0.491 | | | | A-B | N/D |
| E) MS 60 mg/NTX 0.01 mg | 23 | 0.17 | 0.491 | | | | A-C | N/D |
| F) MS 60 mg/NTX 0.1 mg | 22 | 0.18 | 0.501 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 30 MINUTES | | | | | | | | |
| A) Placebo | 19 | 0.32 | 0.478 | | | | Treatment | 0.165 |
| B) MS 60 mg | 25 | 0.16 | 0.374 | | | | Site | 0.182 |
| C) NTX 0.01 mg | 21 | 0.24 | 0.539 | | | | Treatment by Site | 0.038* |
| D) MS 60 mg/NTX 0.001 mg | 32 | 0.25 | 0.508 | | | | A-B | N/D |
| E) MS 60 mg/NTX 0.01 mg | 23 | 0.52 | 0.846 | | | | A-C | N/D |
| F) MS 60 mg/NTX 0.1 mg | 22 | 0.41 | 0.666 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 45 MINUTES | | | | | | | | |
| A) Placebo | 19 | 0.42 | 0.607 | | | | Treatment | 0.195 |
| B) MS 60 mg | 25 | 0.40 | 0.577 | | | | Site | 0.857 |
| C) NTX 0.01 mg | 21 | 0.33 | 0.658 | | | | Treatment by Site | 0.281 |
| D) MS 60 mg/NTX 0.001 mg | 32 | 0.47 | 0.803 | | | | A-B | N/D |
| E) MS 60 mg/NTX 0.01 mg | 23 | 0.87 | 1.140 | | | | A-C | N/D |
| F) MS 60 mg/NTX 0.1 mg | 22 | 0.73 | 1.032 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 1 HOUR | | | | | | | | |
| A) Placebo | 19 | 0.47 | 0.612 | | | | Treatment | 0.137 |
| B) MS 60 mg | 25 | 0.52 | 0.714 | | | | Site | 0.553 |
| C) NTX 0.01 mg | 21 | 0.48 | 0.873 | | | | Treatment by Site | 0.297 |
| D) MS 60 mg/NTX 0.001 mg | 32 | 0.56 | 0.948 | | | | A-B | N/D |
| E) MS 60 mg/NTX 0.01 mg | 23 | 0.96 | 1.147 | | | | A-C | N/D |
| F) MS 60 mg/NTX 0.1 mg | 22 | 1.14 | 1.320 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 1.5 HOURS | | | | | | | | |
| A) Placebo | 19 | 0.58 | 0.838 | | | | Treatment | 0.024* |
| B) MS 60 mg | 25 | 0.68 | 0.852 | | | | Site | 0.719 |
| C) NTX 0.01 mg | 21 | 0.38 | 0.740 | | | | Treatment by Site | 0.448 |
| D) MS 60 mg/NTX 0.001 mg | 32 | 0.81 | 1.091 | | | | A-B | 0.841 |
| E) MS 60 mg/NTX 0.01 mg | 23 | 1.17 | 1.302 | | | | A-C | 0.479 |
| F) MS 60 mg/NTX 0.1 mg | 22 | 1.45 | 1.371 | | | | A-D | 0.607 |
| | | | | | | | A-E | 0.086 |
| | | | | | | | A-F | 0.026* |
| | | | | | | | B-C | 0.334 |
| | | | | | | | B-D | 0.739 |
| | | | | | | | B-E | 0.102 |
| | | | | | | | B-F | 0.028* |
| | | | | | | | C-D | 0.184 |
| | | | | | | | C-E | 0.012* |
| | | | | | | | C-F | 0.002** |
| | | | | | | | D-E | 0.161 |
| | | | | | | | D-F | 0.047* |
| | | | | | | | E-F | 0.576 |
| 2 HOURS | | | | | | | | |
| A) Placebo | 19 | 0.58 | 0.838 | | | | Treatment | 0.005** |
| B) MS 60 mg | 25 | 0.60 | 0.764 | | | | Site | 0.289 |
| C) NTX 0.01 mg | 21 | 0.33 | 0.658 | | | | Treatment by Site | 0.160 |
| D) MS 60 mg/NTX 0.001 mg | 32 | 0.94 | 1.134 | | | | A-B | 0.939 |
| E) MS 60 mg/NTX 0.01 mg | 23 | 1.09 | 1.311 | | | | A-C | 0.401 |
| F) MS 60 mg/NTX 0.1 mg | 22 | 1.64 | 1.497 | | | | A-D | 0.418 |
| | | | | | | | A-E | 0.147 |
| | | | | | | | A-F | 0.007** |
| | | | | | | | B-C | 0.410 |
| | | | | | | | B-D | 0.333 |
| | | | | | | | B-E | 0.102 |
| | | | | | | | B-F | 0.003** |
| | | | | | | | C-D | 0.075 |
| | | | | | | | C-E | 0.018* |
| | | | | | | | C-F | <0.001*** |
| | | | | | | | D-E | 0.430 |
| | | | | | | | D-F | 0.029* |
| | | | | | | | E-F | 0.191 |
| 3 HOURS | | | | | | | | |
| A) Placebo | 19 | 0.74 | 1.046 | | | | Treatment | 0.006** |
| B) MS 60 mg | 25 | 0.64 | 0.810 | | | | Site | 0.283 |
| C) NTX 0.01 mg | 21 | 0.33 | 0.730 | | | | Treatment by Site | 0.431 |
| D) MS 60 mg/NTX 0.001 mg | 32 | 1.00 | 1.295 | | | | A-B | 0.713 |
| E) MS 60 mg/NTX 0.01 mg | 23 | 1.30 | 1.428 | | | | A-C | 0.242 |
| F) MS 60 mg/NTX 0.1 mg | 22 | 1.73 | 1.486 | | | | A-D | 0.606 |
| | | | | | | | A-E | 0.166 |
| | | | | | | | A-F | 0.023* |
| | | | | | | | B-C | 0.380 |
| | | | | | | | B-D | 0.328 |
| | | | | | | | B-E | 0.062 |
| | | | | | | | B-F | 0.005** |
| | | | | | | | C-D | 0.065 |
| | | | | | | | C-E | 0.008** |
| | | | | | | | C-F | <0.001*** |
| | | | | | | | D-E | 0.305 |
| | | | | | | | D-F | 0.042* |
| | | | | | | | E-F | 0.340 |
| 4 HOURS | | | | | | | | |
| A) Placebo | 19 | 0.89 | 1.197 | | | | Treatment | 0.007** |
| B) MS 60 mg | 25 | 0.76 | 1.052 | | | | Site | 0.235 |
| C) NTX 0.01 mg | 21 | 0.38 | 1.805 | | | | Treatment by Site | 0.349 |
| D) MS 60 mg/NTX 0.001 mg | 32 | 1.13 | 1.338 | | | | A-B | 0.685 |
| E) MS 60 mg/NTX 0.01 mg | 23 | 1.39 | 1.469 | | | | A-C | 0.184 |
| F) MS 60 mg/NTX 0.1 mg | 22 | 1.95 | 1.647 | | | | A-D | 0.705 |
| | | | | | | | A-E | 0.283 |
| | | | | | | | A-F | 0.026* |
| | | | | | | | B-C | 0.314 |
| | | | | | | | B-D | 0.383 |
| | | | | | | | B-E | 0.115 |
| | | | | | | | B-F | 0.005** |
| | | | | | | | C-D | 0.060 |
| | | | | | | | C-E | 0.013* |
| | | | | | | | C-F | <0.001*** |
| | | | | | | | D-E | 0.415 |
| | | | | | | | D-F | 0.033* |
| | | | | | | | E-F | 0.219 |
| 5 HOURS | | | | | | | | |
| A) Placebo | 19 | 0.84 | 1.167 | | | | Treatment | 0.019* |
| B) MS 60 mg | 25 | 0.80 | 1.118 | | | | Site | 0.277 |
| C) NTX 0.01 mg | 21 | 0.38 | 0.805 | | | | Treatment by Site | 0.200 |
| D) MS 60 mg/NTX 0.001 mg | 32 | 1.19 | 1.424 | | | | A-B | 0.864 |
| E) MS 60 mg/NTX 0.01 mg | 23 | 1.43 | 1.532 | | | | A-C | 0.236 |
| F) MS 60 mg/NTX 0.1 mg | 22 | 1.86 | 1.670 | | | | A-D | 0.514 |
| | | | | | | | A-E | 0.199 |
| | | | | | | | A-F | 0.044* |
| | | | | | | | B-C | 0.273 |
| | | | | | | | B-D | 0.366 |
| | | | | | | | B-E | 0.119 |
| | | | | | | | B-F | 0.019* |
| | | | | | | | C-D | 0.045* |
| | | | | | | | C-E | 0.011* |
| | | | | | | | C-F | 0.001** |
| | | | | | | | D-E | 0.442 |
| | | | | | | | D-F | 0.109 |
| | | | | | | | E-F | 0.434 |
| 6 HOURS | | | | | | | | |
| A) Placebo | 19 | 0.89 | 1.286 | | | | Treatment | 0.009** |
| B) MS 60 mg | 25 | 0.76 | 1.052 | | | | Site | 0.197 |
| C) NTX 0.01 mg | 21 | 0.33 | 0.730 | | | | Treatment by Site | 0.276 |
| D) MS 60 mg/NTX 0.001 mg | 32 | 1.19 | 1.469 | | | | A-B | 0.713 |
| E) MS 60 mg/NTX 0.01 mg | 23 | 1.22 | 1.445 | | | | A-C | 0.162 |
| F) MS 60 mg/NTX 0.1 mg | 22 | 2.00 | 1.746 | | | | A-D | 0.617 |
| | | | | | | | A-E | 0.547 |
| | | | | | | | A-F | 0.025* |
| | | | | | | | B-C | 0.262 |
| | | | | | | | B-D | 0.336 |
| | | | | | | | B-E | 0.303 |
| | | | | | | | B-F | 0.005** |
| | | | | | | | C-D | 0.037* |
| | | | | | | | C-E | 0.038* |
| | | | | | | | C-F | <0.001*** |
| | | | | | | | D-E | 0.877 |
| | | | | | | | D-F | 0.044* |
| | | | | | | | E-F | 0.084 |
| 7 HOURS | | | | | | | | |
| A) Placebo | 19 | 0.84 | 1.167 | | | | Treatment | 0.008** |
| B) MS 60 mg | 25 | 0.80 | 1.118 | | | | Site | 0.211 |
| C) NTX 0.01 mg | 21 | 0.38 | 0.805 | | | | Treatment by Site | 0.270 |
| D) MS 60 mg/NTX 0.001 mg | 32 | 1.16 | 1.439 | | | | A-B | 0.901 |
| E) MS 60 mg/NTX 0.01 mg | 23 | 1.39 | 1.616 | | | | A-C | 0.268 |
| F) MS 60 mg/NTX 0.1 mg | 22 | 2.05 | 1.786 | | | | A-D | 0.584 |
| | | | | | | | A-E | 0.230 |
| | | | | | | | A-F | 0.015* |
| | | | | | | | B-C | 0.289 |
| | | | | | | | B-D | 0.461 |
| | | | | | | | B-E | 0.156 |
| | | | | | | | B-F | 0.006** |
| | | | | | | | C-D | 0.070 |
| | | | | | | | C-E | 0.017* |
| | | | | | | | C-F | <0.001*** |
| | | | | | | | D-E | 0.434 |
| | | | | | | | D-F | 0.030* |
| | | | | | | | E-F | 0.196 |
| 8 HOURS | | | | | | | | |
| A) Placebo | 19 | 0.89 | 1.286 | | | | Treatment | 0.009** |
| B) MS 60 mg | 25 | 0.80 | 1.118 | | | | Site | 0.217 |
| C) NTX 0.01 mg | 21 | 0.33 | 0.730 | | | | Treatment by Site | 0.259 |
| D) MS 60 mg/NTX 0.001 mg | 32 | 1.13 | 1.431 | | | | A-B | 0.784 |
| E) MS 60 mg/NTX 0.01 mg | 23 | 1.39 | 1.616 | | | | A-C | 0.172 |
| F) MS 60 mg/NTX 0.1 mg | 22 | 2.00 | 1.746 | | | | A-D | 0.767 |
| | | | | | | | A-E | 0.290 |
| | | | | | | | A-F | 0.028* |
| | | | | | | | B-C | 0.236 |
| | | | | | | | B-D | 0.526 |
| | | | | | | | B-E | 0.155 |
| | | | | | | | B-F | 0.008** |
| | | | | | | | C-D | 0.065 |
| | | | | | | | C-E | 0.012* |
| | | | | | | | C-F | <0.001*** |
| | | | | | | | D-E | 0.376 |
| | | | | | | | D-F | 0.030* |
| | | | | | | | E-F | 0.228 |

[1] P-Values are from two-way analysis of variance and its contrasts with treatment, site, and treatment by site interaction as factors.
*, **, ***P-Value <=0.05, <=0.01, or <=0.001 respectively.
N/D: Not done (because overall P-Value not significant).

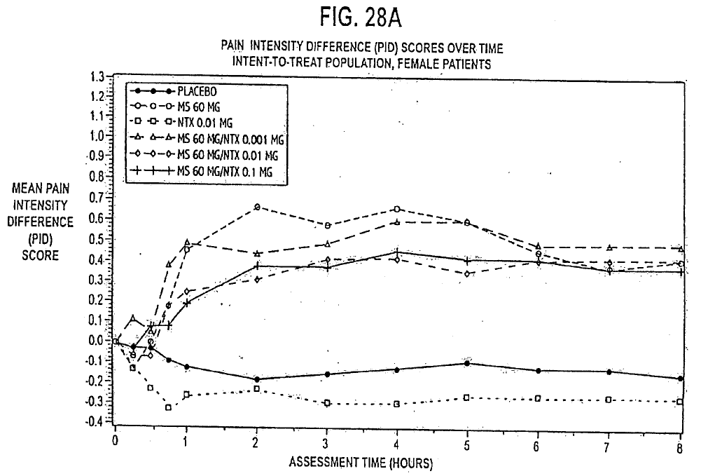

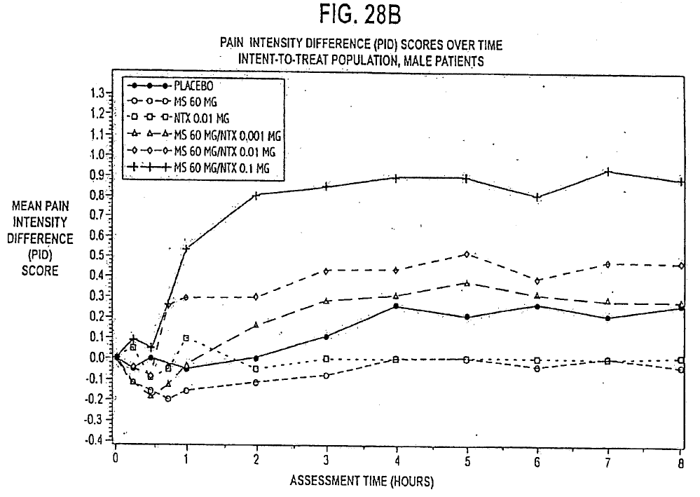

The hourly pain intensity difference (PID) data presented in Table 49A and FIG. 28A for females and Table 49B and FIG. 28B for males. In females, the mean PD scores for 45 minutes to 8 hours are higher for all three NTX combination groups and the MS group than for the placebo group. In males, all three NTX combination groups have higher mean PID scores than the placebo and MS alone groups for 45 minutes to 8 hours. The 0.1 mg NTX combination group has the highest mean PID scores.

TABLE 49A
Pain Intensity Difference (PID) Scores
Intent-To-Treat Population, Female Patients

| | PAIN INTENSITY | | | | | | | |
|---|---|---|---|---|---|---|---|---|
| TIME | DIFFERENCE SCORE (PID) | | | | | | | |
| TREATMENT | N | MEAN | SD | MIN | MEDIAN | MAX | SOURCE | P-VALUE [1] |
| 15 MINUTES | | | | | | | | |
| A) Placebo | 32 | −0.03 | 0.309 | | | | Treatment | 0.444 |
| B) MS 60 mg | 28 | −0.14 | 0.356 | | | | Site | 0.158 |
| C) NTX 0.01 mg | 30 | −0.13 | 0.434 | | | | Treatment By Site | 0.088 |
| D) MS 60 mg/NTX 0.001 mg | 18 | 0.11 | 0.323 | | | | A-B | N/D |
| E) MS 60 mg/NTX 0.01 mg | 28 | −0.07 | 0.663 | | | | A-C | N/D |
| F) MS 60 mg/NTX 0.1 mg | 26 | −0.04 | 0.445 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 30 MINUTES | | | | | | | | |
| A) Placebo | 32 | −0.03 | 0.400 | | | | Treatment | 0.388 |
| B) MS 60 mg | 28 | 0.00 | 0.544 | | | | Site | 0.116 |
| C) NTX 0.01 mg | 30 | −0.23 | 0.626 | | | | Treatment By Site | 0.333 |
| D) MS 60 mg/NTX 0.001 mg | 18 | 0.06 | 0.236 | | | | A-B | N/D |
| E) MS 60 mg/NTX 0.01 mg | 28 | −0.07 | 0.858 | | | | A-C | N/D |
| F) MS 60 mg/NTX 0.1 mg | 26 | 0.08 | 0.560 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 45 MINUTES | | | | | | | | |
| A) Placebo | 32 | −0.09 | 0.390 | | | | Treatment | 0.004** |
| B) MS 60 mg | 28 | 0.18 | 0.670 | | | | Site | 0.061 |
| C) NTX 0.01 mg | 30 | −0.33 | 0.606 | | | | Treatment By Site | 0.289 |
| D) MS 60 mg/NTX 0.001 mg | 18 | 0.39 | 0.778 | | | | A-B | 0.115 |
| E) MS 60 mg/NTX 0.01 mg | 28 | 0.18 | 0.945 | | | | A-C | 0.215 |
| F) MS 60 mg/NTX 0.1 mg | 26 | 0.08 | 0.628 | | | | A-D | 0.005** |
| | | | | | | | A-E | 0.184 |
| | | | | | | | A-F | 0.278 |
| | | | | | | | B-C | 0.007** |
| | | | | | | | B-D | 0.170 |
| | | | | | | | B-E | 0.789 |
| | | | | | | | B-F | 0.647 |
| | | | | | | | C-D | <0.001*** |
| | | | | | | | C-E | 0.013* |
| | | | | | | | C-F | 0.027* |
| | | | | | | | D-E | 0.106 |
| | | | | | | | D-F | 0.079 |
| | | | | | | | E-F | 0.841 |
| 1 HOUR | | | | | | | | |
| A) Placebo | 32 | −0.13 | 0.421 | | | | Treatment | <0.001*** |
| B) MS 60 mg | 28 | 0.46 | 0.744 | | | | Site | 0.045* |
| C) NTX 0.01 mg | 30 | −0.27 | 0.691 | | | | Treatment By Site | 0.422 |
| D) MS 60 mg/NTX 0.001 mg | 18 | 0.50 | 0.786 | | | | A-B | <0.001*** |
| E) MS 60 mg/NTX 0.01 mg | 28 | 0.25 | 0.844 | | | | A-C | 0.508 |
| F) MS 60 mg/NTX 0.1 mg | 26 | 0.19 | 0.634 | | | | A-D | 0.001** |
| | | | | | | | A-E | 0.064 |
| | | | | | | | A-F | 0.070 |
| | | | | | | | B-C | <0.001*** |
| | | | | | | | B-D | 0.760 |
| | | | | | | | B-E | 0.127 |
| | | | | | | | B-F | 0.141 |
| | | | | | | | C-D | <0.001*** |
| | | | | | | | C-E | 0.015* |
| | | | | | | | C-F | 0.018* |
| | | | | | | | D-E | 0.101 |
| | | | | | | | D-F | 0.111 |
| | | | | | | | E-F | 0.991 |
| 1.5 HOURS | | | | | | | | |
| A) Placebo | 32 | −0.16 | 0.574 | | | | Treatment | <0.001*** |
| B) MS 60 mg | 28 | 0.57 | 0.690 | | | | Site | 0.172 |
| C) NTX 0.01 mg | 30 | −0.23 | 0.679 | | | | Treatment By Site | 0.300 |
| D) MS 60 mg/NTX 0.001 mg | 18 | 0.44 | 0.922 | | | | A-B | <0.001*** |
| E) MS 60 mg/NTX 0.01 mg | 28 | 0.36 | 0.870 | | | | A-C | 0.772 |
| F) MS 60 mg/NTX 0.1 mg | 26 | 0.31 | 0.736 | | | | A-D | 0.001** |
| | | | | | | | A-E | 0.012* |
| | | | | | | | A-F | 0.031* |
| | | | | | | | B-C | <0.001*** |
| | | | | | | | B-D | 0.943 |
| | | | | | | | B-E | 0.205 |
| | | | | | | | B-F | 0.133 |
| | | | | | | | C-D | <0.001*** |
| | | | | | | | C-E | 0.007** |
| | | | | | | | C-F | 0.018* |
| | | | | | | | D-E | 0.301 |
| | | | | | | | D-F | 0.211 |
| | | | | | | | E-F | 0.783 |
| 2 HOURS | | | | | | | | |
| A) Placebo | 32 | −0.19 | 0.644 | | | | Treatment | <0.001*** |
| B) MS 60 mg | 28 | 0.68 | 0.905 | | | | Site | 0.121 |
| C) NTX 0.01 mg | 30 | −0.23 | 0.679 | | | | Treatment By Site | 0.232 |
| D) MS 60 mg/NTX 0.001 mg | 18 | 0.44 | 1.097 | | | | A-B | <0.001*** |
| E) MS 60 mg/NTX 0.01 mg | 28 | 0.32 | 0.863 | | | | A-C | 0.934 |
| F) MS 60 mg/NTX 0.1 mg | 26 | 0.38 | 0.804 | | | | A-D | 0.001** |
| | | | | | | | A-E | 0.022* |
| | | | | | | | A-F | 0.013* |
| | | | | | | | B-C | <0.001*** |
| | | | | | | | B-D | 0.756 |
| | | | | | | | B-E | 0.080 |
| | | | | | | | B-F | 0.144 |
| | | | | | | | C-D | 0.001** |
| | | | | | | | C-E | 0.022* |
| | | | | | | | C-F | 0.013* |
| | | | | | | | D-E | 0.224 |
| | | | | | | | D-F | 0.329 |
| | | | | | | | E-F | 0.803 |
| 3 HOURS | | | | | | | | |
| A) Placebo | 32 | −0.16 | 0.723 | | | | Treatment | <0.001*** |
| B) MS 60 mg | 28 | 0.59 | 0.872 | | | | Site | 0.165 |
| C) NTX 0.01 mg | 30 | −0.30 | 0.651 | | | | Treatment By Site | 0.321 |
| D) MS 60 mg/NTX 0.001 mg | 18 | 0.50 | 1.098 | | | | A-B | <0.001*** |
| E) MS 60 mg/NTX 0.01 mg | 28 | 0.43 | 0.920 | | | | A-C | 0.551 |
| F) MS 60 mg/NTX 0.1 mg | 26 | 0.38 | 0.804 | | | | A-D | 0.001** |
| | | | | | | | A-E | 0.011* |
| | | | | | | | A-F | 0.024* |
| | | | | | | | B-C | <0.001*** |
| | | | | | | | B-D | 0.838 |
| | | | | | | | B-E | 0.392 |
| | | | | | | | B-F | 0.300 |
| | | | | | | | C-D | <0.001*** |
| | | | | | | | C-E | 0.002** |
| | | | | | | | C-F | 0.006** |
| | | | | | | | D-E | 0.340 |
| | | | | | | | D-F | 0.266 |
| | | | | | | | E-F | 0.835 |
| 4 HOURS | | | | | | | | |
| A) Placebo | 32 | −0.13 | 0.751 | | | | Treatment | <0.001*** |
| B) MS 60 mg | 28 | 0.68 | 1.020 | | | | Site | 0.458 |
| C) NTX 0.01 mg | 30 | −0.30 | 0.651 | | | | Treatment By Site | 0.517 |
| D) MS 60 mg/NTX 0.001 mg | 18 | 0.61 | 1.195 | | | | A-B | 0.001** |
| E) MS 60 mg/NTX 0.01 mg | 28 | 0.43 | 0.920 | | | | A-C | 0.509 |
| F) MS 60 mg/NTX 0.1 mg | 26 | 0.46 | 0.905 | | | | A-D | 0.002** |
| | | | | | | | A-E | 0.025* |
| | | | | | | | A-F | 0.025* |
| | | | | | | | B-C | <0.001*** |
| | | | | | | | B-D | 0.816 |
| | | | | | | | B-E | 0.282 |
| | | | | | | | B-F | 0.322 |
| | | | | | | | C-D | <0.001*** |
| | | | | | | | C-E | 0.005** |
| | | | | | | | C-F | 0.005** |
| | | | | | | | D-E | 0.241 |
| | | | | | | | D-F | 0.272 |
| | | | | | | | E-F | 0.953 |
| 5 HOURS | | | | | | | | |
| A) Placebo | 32 | −0.09 | 0.818 | | | | Treatment | <0.001*** |
| B) MS 60 mg | 28 | 0.61 | 0.994 | | | | Site | 0.789 |
| C) NTX 0.01 mg | 30 | −0.27 | 0.640 | | | | Treatment By Site | 0.311 |
| D) MS 60 mg/NTX 0.001 mg | 18 | 0.61 | 1.195 | | | | A-B | 0.004** |
| E) MS 60 mg/NTX 0.01 mg | 28 | 0.36 | 0.911 | | | | A-C | 0.501 |
| F) MS 60 mg/NTX 0.1 mg | 26 | 0.42 | 0.857 | | | | A-D | 0.002** |
| | | | | | | | A-E | 0.065 |
| | | | | | | | A-F | 0.061 |
| | | | | | | | B-C | <0.001*** |
| | | | | | | | B-D | 0.612 |
| | | | | | | | B-E | 0.287 |
| | | | | | | | B-F | 0.335 |
| | | | | | | | C-D | <0.001*** |
| | | | | | | | C-E | 0.015* |
| | | | | | | | C-F | 0.015* |
| | | | | | | | D-E | 0.150 |
| | | | | | | | D-F | 0.178 |
| | | | | | | | E-F | 0.939 |
| 6 HOURS | | | | | | | | |
| A) Placebo | 32 | −0.13 | 0.751 | | | | Treatment | 0.004** |
| B) MS 60 mg | 28 | 0.46 | 0.962 | | | | Site | 0.666 |
| C) NTX 0.01 mg | 30 | −0.27 | 0.640 | | | | Treatment By Site | 0.562 |
| D) MS 60 mg/NTX 0.001 mg | 18 | 0.50 | 1.150 | | | | A-B | 0.016* |
| E) MS 60 mg/NTX 0.01 mg | 28 | 0.43 | 1.034 | | | | A-C | 0.612 |
| F) MS 60 mg/NTX 0.1 mg | 26 | 0.42 | 0.857 | | | | A-D | 0.010* |
| | | | | | | | A-E | 0.024* |
| | | | | | | | A-F | 0.043* |
| | | | | | | | B-C | 0.005** |
| | | | | | | | B-D | 0.641 |
| | | | | | | | B-E | 0.859 |
| | | | | | | | B-F | 0.729 |
| | | | | | | | C-D | 0.003** |
| | | | | | | | C-E | 0.007** |
| | | | | | | | C-F | 0.015* |
| | | | | | | | D-E | 0.530 |
| | | | | | | | D-F | 0.444 |
| | | | | | | | E-F | 0.860 |
| 7 HOURS | | | | | | | | |
| A) Placebo | 32 | −0.13 | 0.751 | | | | Treatment | 0.005** |
| B) MS 60 mg | 28 | 0.39 | 0.956 | | | | Site | 0.810 |
| C) NTX 0.01 mg | 30 | −0.27 | 0.640 | | | | Treatment By Site | 0.600 |
| D) MS 60 mg/NTX 0.001 mg | 18 | 0.50 | 1.150 | | | | A-B | 0.028* |
| E) MS 60 mg/NTX 0.01 mg | 28 | 0.43 | 1.034 | | | | A-C | 0.608 |
| F) MS 60 mg/NTX 0.1 mg | 26 | 0.38 | 0.804 | | | | A-D | 0.010* |
| | | | | | | | A-E | 0.022* |
| | | | | | | | A-F | 0.056 |
| | | | | | | | B-C | 0.009** |
| | | | | | | | B-D | 0.505 |
| | | | | | | | B-E | 0.961 |
| | | | | | | | B-F | 0.801 |
| | | | | | | | C-D | 0.003** |
| | | | | | | | C-E | 0.007** |
| | | | | | | | C-F | 0.020* |
| | | | | | | | D-E | 0.527 |
| | | | | | | | D-F | 0.378 |
| | | | | | | | E-F | 0.761 |
| 8 HOURS | | | | | | | | |
| A) Placebo | 32 | −0.16 | 0.677 | | | | Treatment | 0.002** |
| B) MS 60 mg | 28 | 0.43 | 0.997 | | | | Site | 0.945 |
| C) NTX 0.01 mg | 30 | −0.27 | 0.640 | | | | Treatment By Site | 0.562 |
| D) MS 60 mg/NTX 0.001 mg | 18 | 0.50 | 1.150 | | | | A-B | 0.012* |
| E) MS 60 mg/NTX 0.01 mg | 28 | 0.43 | 1.034 | | | | A-C | 0.687 |
| F) MS 60 mg/NTX 0.1 mg | 26 | 0.38 | 0.804 | | | | A-D | 0.007** |
| | | | | | | | A-E | 0.016* |
| | | | | | | | A-F | 0.043* |
| | | | | | | | B-C | 0.005** |
| | | | | | | | B-D | 0.622 |
| | | | | | | | B-E | 0.875 |
| | | | | | | | B-F | 0.650 |
| | | | | | | | C-D | 0.003** |
| | | | | | | | C-E | 0.007** |
| | | | | | | | C-F | 0.020* |
| | | | | | | | D-E | 0.525 |
| | | | | | | | D-F | 0.376 |
| | | | | | | | E-F | 0.760 |

[1] P-Values are from two-way analysis of variance and its contrasts with treatment, site, and treatment by site interaction as factors.
*, **, ***P-Value <=0.05, <=0.01, or <=0.001 respectively.
N/D: Not done (because overall p-value not significant).

TABLE 49B
Pain Intensity Difference (PID) Scores
Intent-To-Treat Population, Male Patients

| | PAIN INTENSITY | | | | | | | |
|---|---|---|---|---|---|---|---|---|
| TIME | DIFFERENCE SCORE (PR) | | | | | | | |
| TREATMENT | N | MEAN | SD | MIN | MEDIAN | MAX | SOURCE | P-VALUE [1] |
| 15 MINUTES | | | | | | | | |
| A) Placebo | 19 | −0.05 | 0.405 | | | | Treatment | 0.460 |
| B) MS 60 mg | 25 | −0.12 | 0.332 | | | | Site | 0.314 |
| C) NTX 0.01 mg | 21 | 0.05 | 0.384 | | | | Treatment By Site | 0.584 |
| D) MS 60 mg/NTX 0.001 mg | 32 | −0.13 | 0.421 | | | | A-B | N/D |
| E) MS 60 mg/NTX 0.01 mg | 23 | −0.04 | 0.367 | | | | A-C | N/D |
| F) MS 60 mg/NTX 0.1 mg | 22 | 0.09 | 0.526 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 30 MINUTES | | | | | | | | |
| A) Placebo | 19 | 0.00 | 0.471 | | | | Treatment | 0.564 |
| B) MS 60 mg | 25 | −0.16 | 0.374 | | | | Site | 0.389 |
| C) NTX 0.01 mg | 21 | −0.10 | 0.539 | | | | Treatment By Site | 0.422 |
| D) MS 60 mg/NTX 0.001 mg | 32 | −0.19 | 0.644 | | | | A-B | N/D |
| E) MS 60 mg/NTX 0.01 mg | 23 | −0.09 | 0.596 | | | | A-C | N/D |
| F) MS 60 mg/NTX 0.1 mg | 22 | 0.05 | 0.486 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 45 MINUTES | | | | | | | | |
| A) Placebo | 19 | −0.05 | 0.705 | | | | Treatment | 0.170 |
| B) MS 60 mg | 25 | −0.20 | 0.577 | | | | Site | 0.056 |
| C) NTX 0.01 mg | 21 | −0.05 | 0.590 | | | | Treatment By Site | 0.622 |
| D) MS 60 mg/NTX 0.001 mg | 32 | −0.13 | 0.751 | | | | A-B | N/D |
| E) MS 60 mg/NTX 0.01 mg | 23 | 0.26 | 0.964 | | | | A-C | N/D |
| F) MS 60 mg/NTX 0.1 mg | 22 | 0.27 | 0.827 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 1 HOUR | | | | | | | | |
| A) Placebo | 19 | −0.05 | 0.705 | | | | Treatment | 0.068 |
| B) MS 60 mg | 25 | −0.16 | 0.554 | | | | Site | 0.032* |
| C) NTX 0.01 mg | 21 | 0.10 | 0.768 | | | | Treatment By Site | 0.660 |
| D) MS 60 mg/NTX 0.001 mg | 32 | −0.03 | 0.861 | | | | A-B | N/D |
| E) MS 60 mg/NTX 0.01 mg | 23 | 0.30 | 0.974 | | | | A-C | N/D |
| F) MS 60 mg/NTX 0.1 mg | 22 | 0.55 | 0.963 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 1.5 HOURS | | | | | | | | |
| A) Placebo | 19 | 0.05 | 0.705 | | | | Treatment | 0.234 |
| B) MS 60 mg | 25 | −0.04 | 0.676 | | | | Site | 0.128 |
| C) NTX 0.01 mg | 21 | 0.10 | 0.700 | | | | Treatment By Site | 0.611 |
| D) MS 60 mg/NTX 0.001 mg | 32 | 0.06 | 0.948 | | | | A-B | N/D |
| E) MS 60 mg/NTX 0.01 mg | 23 | 0.35 | 0.935 | | | | A-C | N/D |
| F) MS 60 mg/NTX 0.1 mg | 22 | 0.55 | 1.011 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 2 HOURS | | | | | | | | |
| A) Placebo | 19 | 0.00 | 0.745 | | | | Treatment | 0.008** |
| B) MS 60 mg | 25 | −0.12 | 0.600 | | | | Site | 0.022* |
| C) NTX 0.01 mg | 21 | −0.05 | 0.669 | | | | Treatment By Site | 0.182 |
| D) MS 60 mg/NTX 0.001 mg | 32 | 0.16 | 0.884 | | | | A-B | 0.541 |
| E) MS 60 mg/NTX 0.01 mg | 23 | 0.30 | 0.926 | | | | A-C | 0.796 |
| F) MS 60 mg/NTX 0.1 mg | 22 | 0.82 | 1.097 | | | | A-D | 0.745 |
| | | | | | | | A-E | 0.291 |
| | | | | | | | A-F | 0.007** |
| | | | | | | | B-C | 0.722 |
| | | | | | | | B-D | 0.295 |
| | | | | | | | B-E | 0.077 |
| | | | | | | | B-F | <0.001*** |
| | | | | | | | C-D | 0.530 |
| | | | | | | | C-E | 0.175 |
| | | | | | | | C-F | 0.002** |
| | | | | | | | D-E | 0.394 |
| | | | | | | | D-F | 0.006** |
| | | | | | | | E-F | 0.080 |
| 3 HOURS | | | | | | | | |
| A) Placebo | 19 | 0.11 | 0.875 | | | | Treatment | 0.032* |
| B) MS 60 mg | 25 | −0.08 | 0.702 | | | | Site | 0.009** |
| C) NTX 0.01 mg | 21 | 0.00 | 0.707 | | | | Treatment By Site | 0.479 |
| D) MS 60 mg/NTX 0.001 mg | 32 | 0.28 | 1.054 | | | | A-B | 0.465 |
| B) MS 60 mg/NTX 0.01 mg | 23 | 0.43 | 1.037 | | | | A-C | 0.704 |
| F) MS 60 mg/NTX 0.1 mg | 22 | 0.86 | 1.167 | | | | A-D | 0.668 |
| | | | | | | | A-E | 0.325 |
| | | | | | | | A-F | 0.027* |
| | | | | | | | B-C | 0.727 |
| | | | | | | | B-D | 0.196 |
| | | | | | | | B-E | 0.069 |
| | | | | | | | B-F | 0.001** |
| | | | | | | | C-D | 0.383 |
| | | | | | | | C-E | 0.158 |
| | | | | | | | C-F | 0.007** |
| | | | | | | | D-E | 0.507 |
| | | | | | | | D-F | 0.040* |
| | | | | | | | E-F | 0.194 |
| 4 HOURS | | | | | | | | |
| A) Placebo | 19 | 0.26 | 1.046 | | | | Treatment | 0.084 |
| B) MS 60 mg | 25 | 0.00 | 0.764 | | | | Site | 0.035* |
| C) NTX 0.01 mg | 21 | 0.00 | 0.707 | | | | Treatment By Site | 0.369 |
| D) MS 60 mg/NTX 0.001 mg | 32 | 0.31 | 1.061 | | | | A-B | N/D |
| E) MS 60 mg/NTX 0.01 mg | 23 | 0.43 | 1.037 | | | | A-C | N/D |
| F) MS 60 mg/NTX 0.1 mg | 22 | 0.91 | 1.342 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 5 HOURS | | | | | | | | |
| A) Placebo | 19 | 0.21 | 0.976 | | | | Treatment | 0.078 |
| B) MS 60 mg | 25 | 0.00 | 0.764 | | | | Site | 0.020* |
| C) NTX 0.01 mg | 21 | 0.00 | 0.707 | | | | Treatment By Site | 0.274 |
| D) MS 60 mg/NTX 0.001 mg | 32 | 0.38 | 1.100 | | | | A-B | N/D |
| E) MS 60 mg/NTX 0.01 mg | 23 | 0.52 | 1.123 | | | | A-C | N/D |
| F) MS 60 mg/NTX 0.1 mg | 22 | 0.91 | 1.342 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 6 HOURS | | | | | | | | |
| A) Placebo | 19 | 0.26 | 1.098 | | | | Treatment | 0.158 |
| B) MS 60 mg | 25 | −0.04 | 0.676 | | | | Site | 0.016* |
| C) NTX 0.01 mg | 21 | 0.00 | 0.707 | | | | Treatment By Site | 0.231 |
| D) MS 60 mg/NTX 0.001 mg | 32 | 0.31 | 1.061 | | | | A-B | N/D |
| E) MS 60 mg/NTX 0.01 mg | 23 | 0.39 | 1.118 | | | | A-C | N/D |
| F) MS 60 mg/NTX 0.1 mg | 22 | 0.82 | 1.296 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 7 HOURS | | | | | | | | |
| A) Placebo | 19 | 0.21 | 1.032 | | | | Treatment | 0.058 |
| B) MS 60 mg | 25 | 0.00 | 0.764 | | | | Site | 0.015* |
| C) NTX 0.01 mg | 21 | 0.00 | 0.707 | | | | Treatment By Site | 0.438 |
| D) MS 60 mg/NTX 0.001 mg | 32 | 0.28 | 1.023 | | | | A-B | N/D |
| E) MS 60 mg/NTX 0.01 mg | 23 | 0.48 | 1.201 | | | | A-C | N/D |
| F) MS 60 mg/NTX 0.1 mg | 22 | 0.95 | 1.362 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |
| 8 HOURS | | | | | | | | |
| A) Placebo | 19 | 0.26 | 1.098 | | | | Treatment | 0.064 |
| B) MS 60 mg | 25 | −0.04 | 0.735 | | | | Site | 0.020* |
| C) NTX 0.01 mg | 21 | 0.00 | 0.707 | | | | Treatment By Site | 0.494 |
| D) MS 60 mg/NTX 0.001 mg | 32 | 0.28 | 1.023 | | | | A-B | N/D |
| E) MS 60 mg/NTX 0.01 mg | 23 | 0.48 | 1.201 | | | | A-C | N/D |
| F) MS 60 mg/NTX 0.1 mg | 22 | 0.91 | 1.306 | | | | A-D | N/D |
| | | | | | | | A-E | N/D |
| | | | | | | | A-F | N/D |
| | | | | | | | B-C | N/D |
| | | | | | | | B-D | N/D |
| | | | | | | | B-E | N/D |
| | | | | | | | B-F | N/D |
| | | | | | | | C-D | N/D |
| | | | | | | | C-E | N/D |
| | | | | | | | C-F | N/D |
| | | | | | | | D-E | N/D |
| | | | | | | | D-F | N/D |
| | | | | | | | E-F | N/D |

[1] P-Values are from two-way analysis of variance and its contrasts with treatment, site, and treatment by site interaction as factors.
*, **, ***P-Value <=0.05, <=0.01, or <=0.001 respectively.
N/D: Not done (because overall p-value not significant).

Tables 50A and 50B for females and Tables 50C and 50D for males present the mean MAXPAR and PEAKPID scores. In females, the mean MAXPAR and PEAKPID scores were higher for the MS alone and the NTX combination groups than for the placebo group. In males, the three NTX combination groups had higher mean MAXPAR and PEAKPID scores than the placebo or MS alone groups. The 0.1 mg NTX combination group had the highest mean score for MAXPAR and PEAKPID.

Tables 51A for females and 51B for males present the summary and analysis of global evaluations. For both females and males, the placebo treatment had the highest number of subjects who had poor global evaluation scores based on subject evaluation. For females, the morphine and high-dose (0.1 mg NTX) combination groups were most often rated as “excellent.” For males, the mid-dose (0.01 mg NTX) and high-dose (0.1 mg NTX) combination groups were most often rated as “excellent.”

TABLE 50A
Maximum Pain Relief Scores (MAXPAR)
Intent-To-Treat Population, Female Patients

| MAXIMUM PAIN RELIEF SCORE [1] | | | | | | | | |
|---|---|---|---|---|---|---|---|---|
| TREATMENT | N | MEAN | SD | MIN | MEDIAN | MAX | SOURCE | P-VALUE [2] |
| A) Placebo | 32 | 0.75 | 1.107 | 0 | 0.00 | 3 | TREATMENT | <0.001*** |
| B) MS 60 mg | 28 | 2.14 | 1.177 | 0 | 2.50 | 4 | SITE | 0.484 |
| C) NTX 0.01 mg | 30 | 0.63 | 0.850 | 0 | 0.00 | 3 | TREATMENT BY SITE | 0.271 |
| D) MS 60 mg/NTX 0.001 mg | 18 | 1.67 | 1.572 | 0 | 2.00 | 4 | A-B | <0.001*** |
| E) MS 60 mg/NTX 0.01 mg | 28 | 1.61 | 1.370 | 0 | 1.50 | 4 | A-C | 0.684 |
| F) MS 60 mg/NTX 0.1 mg | 26 | 1.85 | 1.084 | 0 | 2.00 | 4 | A-D | 0.003** |
| | | | | | | | A-E | 0.009** |
| | | | | | | | A-F | 0.001** |
| | | | | | | | B-C | <0.001*** |
| | | | | | | | B-D | 0.493 |
| | | | | | | | B-E | 0.098 |
| | | | | | | | B-F | 0.292 |
| | | | | | | | C-D | 0.001** |
| | | | | | | | C-E | 0.003** |
| | | | | | | | C-F | <0.001*** |
| | | | | | | | D-E | 0.450 |
| | | | | | | | D-F | 0.805 |
| | | | | | | | E-F | 0.568 |

[1] Pain Relief (PR) Scores: 0 = None, 1 = A Little, 2 = Some, 3 = A Lot, 4 = Complete.
[2] P-Values are from Two-Way Analysis of Variance and its Contrasts with Treatment, Site, and Treatment by Site Interaction as Factors.
*, **, ***P-VALUE <=0.05, <=0.01, or <=0.001 RESPECTIVELY.

TABLE 50B
Peak Pain Intensity Differences (PEAKPID)
Intent-To-Treat Population, Female Patients

| PEAK PAIN INTENSITY DIFFERENCES (PEAKPID) | | | | | | | | |
|---|---|---|---|---|---|---|---|---|
| TREATMENT | N | MEAN | SD | MIN | MEDIAN | MAX | SOURCE | P-VALUE [1] |
| A) Placebo | 32 | 0.25 | 0.672 | −1 | 0.00 | 2 | TREATMENT | <0.001*** |
| B) MS 60 mg | 28 | 1.04 | 0.881 | −1 | 1.00 | 3 | SITE | 0.707 |
| C) NTX 0.01 mg | 30 | 0.10 | 0.548 | −1 | 0.00 | 1 | TREATMENT BY SITE | 0.384 |
| D) MS 60 mg/NTX 0.001 mg | 18 | 0.89 | 0.963 | 0 | 1.00 | 3 | A-B | <0.001*** |
| E) MS 60 mg/NTX 0.01 mg | 28 | 0.68 | 1.090 | −1 | 0.50 | 3 | A-C | 0.579 |
| F) MS 60 mg/NTX 0.1 mg | 26 | 0.77 | 0.765 | 0 | 1.00 | 2 | A-D | 0.007** |
| | | | | | | | A-E | 0.086 |
| | | | | | | | A-F | 0.038* |
| | | | | | | | B-C | <0.001*** |
| | | | | | | | B-D | 0.728 |
| | | | | | | | B-E | 0.076 |
| | | | | | | | B-F | 0.182 |
| | | | | | | | C-D | 0.002** |
| | | | | | | | C-E | 0.028* |
| | | | | | | | C-F | 0.012* |
| | | | | | | | D-E | 0.231 |
| | | | | | | | D-F | 0.406 |
| | | | | | | | E-F | 0.690 |

[1] P-Values are from Two-Way Analysis of Variance and its Contrasts with Treatment, Site, and Treatment by Site Interaction as Factors.
*, **, ***P-VALUE <=0.05, <=0.01, or <=0.001 RESPECTIVELY.

TABLE 50C
Maximum Pain Relief Scores (MAXPAR)
Intent-To-Treat Population, Male Patients

| MAXIMUM PAIN RELIEF SCORE [1] | | | | | | | | |
|---|---|---|---|---|---|---|---|---|
| TREATMENT | N | MEAN | SD | MIN | MEDIAN | MAX | SOURCE | P-VALUE [2] |
| A) Placebo | 19 | 1.05 | 1.268 | 0 | 1.00 | 4 | TREATMENT | 0.007** |
| B) MS 60 mg | 25 | 1.08 | 1.115 | 0 | 1.00 | 3 | SITE | 0.501 |
| C) NTX 0.01 mg | 21 | 0.62 | 0.973 | 0 | 0.00 | 3 | TREATMENT BY SITE | 0.581 |
| D) MS 60 mg/NTX 0.001 mg | 32 | 1.47 | 1.414 | 0 | 1.00 | 4 | A-B | 0.978 |
| E) MS 60 mg/NTX 0.01 mg | 23 | 1.61 | 1.616 | 0 | 2.00 | 4 | A-C | 0.303 |
| F) MS 60 mg/NTX 0.1 mg | 22 | 2.32 | 1.701 | 0 | 3.00 | 4 | A-D | 0.373 |
| | | | | | | | A-E | 0.255 |
| | | | | | | | A-F | 0.010* |
| | | | | | | | B-C | 0.257 |
| | | | | | | | B-D | 0.348 |
| | | | | | | | B-E | 0.232 |
| | | | | | | | B-F | 0.006** |
| | | | | | | | C-D | 0.038* |
| | | | | | | | C-E | 0.025* |
| | | | | | | | C-F | <0.001*** |
| | | | | | | | D-E | 0.725 |
| | | | | | | | D-F | 0.049* |
| | | | | | | | E-F | 0.132 |

[1] Pain Relief (PR) Scores: 0 = None, 1 = A Little, 2 = Some, 3 = A Lot, 4 = Complete.
[2] P-Values are from Two-Way Analysis of Variance and its Contrasts with Treatment, Site, and Treatment by Site Interaction as Factors.
*, **, ***P-VALUE <=0.05, <=0.01, or <=0.001 RESPECTIVELY.

TABLE 50D
Peak Pain Intensity Differences (PEAKPID)
Intent-To-Treat Population, Male Patients

| PEAK PAIN INTENSITY DIFFERENCES (PEAKPID) | | | | | | | | |
|---|---|---|---|---|---|---|---|---|
| TREATMENT | N | MEAN | SD | MIN | MEDIAN | MAX | SOURCE | P-VALUE [1] |
| A) Placebo | 19 | 0.53 | 1.020 | −1 | 0.00 | 3 | TREATMENT | 0.019* |
| B) MS 60 mg | 25 | 0.20 | 0.707 | −1 | 0.00 | 2 | SITE | 0.080 |
| C) NTX 0.01 mg | 21 | 0.24 | 0.700 | −1 | 0.00 | 2 | TREATMENT BY SITE | 0.583 |
| D) MS 60 mg/NTX 0.001 mg | 32 | 0.63 | 0.907 | −1 | 0.00 | 3 | A-B | 0.236 |
| E) MS 60 mg/NTX 0.01 mg | 23 | 0.74 | 1.054 | −1 | 0.00 | 3 | A-C | 0.303 |
| F) MS 60 mg/NTX 0.1 mg | 22 | 1.18 | 1.181 | −1 | 1.00 | 3 | A-D | 0.863 |
| | | | | | | | A-E | 0.573 |
| | | | | | | | A-F | 0.060 |
| | | | | | | | B-C | 0.903 |
| | | | | | | | B-D | 0.125 |
| | | | | | | | B-E | 0.066 |
| | | | | | | | B-F | 0.001** |
| | | | | | | | C-D | 0.181 |
| | | | | | | | C-E | 0.098 |
| | | | | | | | C-F | 0.002** |
| | | | | | | | D-E | 0.648 |
| | | | | | | | D-F | 0.052 |
| | | | | | | | E-F | 0.165 |

[1] P-Values are from Two-Way Analysis of Variance and its Contrasts with Treatment, Site, and Treatment by Site Interaction as Factors.
*, **, ***P-VALUE <=0.05, <=0.01, or <=0.001 RESPECTIVELY.

TABLE 51A
Global Evaluation of Study Medication
Intent-To-Treat Population, Female Patients

| | | Poor | Fair | Good | Very Good | Excellent | | | | P-Value |
|---|---|---|---|---|---|---|---|---|---|---|
| TREATMENT | N | (0) | (1) | (2) | (3) | (4) | Mean | (SD) | Source | [1] |
| A) Placebo | 32 | 26 (81.3%) | 2 (6.3%) | 3 (9.4%) | 1 (3.1%) | 0 (0.0%) | 0.3 | 0.79 | Treatment | <0.001*** |
| B) MS 60 mg | 27 | 7 (25.9%) | 4 (14.8%) | 7 (25.9%) | 7 (25.9%) | 2 (7.4%) | 1.7 | 1.32 | A-B | <0.001*** |
| C) NTX 0.01 mg | 29 | 26 (89.7%) | 2 (6.9%) | 0 (0.0%) | 1 (3.4%) | 0 (0.0%) | 0.2 | 0.60 | A-C | 0.403 |
| D) MS 60 mg/NTX 0.001 mg | 16 | 8 (50.0%) | 2 (12.5%) | 3 (18.8%) | 2 (12.5%) | 1 (6.3%) | 1.1 | 1.36 | A-D | 0.015* |
| E) MS 60 mg/NTX 0.01 mg | 27 | 9 (33.3%) | 8 (29.6%) | 2 (7.4%) | 7 (25.9%) | 1 (3.7%) | 1.4 | 1.31 | A-E | <0.001*** |
| F) MS 60 mg/NTX 0.1 mg | 26 | 9 (34.6%) | 7 (26.9%) | 3 (11.5%) | 5 (19.2%) | 2 (7.7%) | 1.4 | 1.36 | A-F | 0.001** |
| | | | | | | | | | B-C | <0.001*** |
| | | | | | | | | | B-D | 0.155 |
| | | | | | | | | | B-E | 0.319 |
| | | | | | | | | | B-F | 0.345 |
| | | | | | | | | | C-D | 0.003** |
| | | | | | | | | | C-E | <0.001*** |
| | | | | | | | | | C-F | <0.001*** |
| | | | | | | | | | D-E | 0.564 |
| | | | | | | | | | D-F | 0.546 |
| | | | | | | | | | E-F | 0.997 |

[1] FROM COCHRAN-MANTEL-HAENZEL TEST FOR RAW MEAN SCORES DIFFERENCE, ADJUSTING FOR SITE..
*, **, ***P-VALUE <=0.05, <=0.01, OR <=0.001 RESPECTIVELY.

TABLE 51B
Global Evaluation of Study Medication
Intent-To-Treat Population, Male Patients

| | | Poor | Fair | Good | Very Good | Excellent | | | | P-Value |
|---|---|---|---|---|---|---|---|---|---|---|
| TREATMENT | N | (0) | (1) | (2) | (3) | (4) | Mean | (SD) | Source | [1] |
| A) Placebo | 19 | 14 (73.7%) | 2 (10.5%) | 2 (10.5%) | 1 (5.3%) | 0 (0.0%) | 0.5 | 0.90 | Treatment | <0.001*** |
| B) MS 60 mg | 25 | 18 (72.0%) | 3 (12.0%) | 4 (16.0%) | 0 (0.0%) | 0 (0.0%) | 0.4 | 0.77 | A-B | 0.891 |
| C) NTX 0.01 mg | 21 | 19 (90.5%) | 1 (4.8%) | 0 (0.0%) | 0 (0.0%) | 1 (4.8%) | 0.2 | 0.89 | A-C | 0.432 |
| D) MS 60 mg/NTX 0.001 mg | 31 | 18 (58.1%) | 4 (12.9%) | 2 (6.5%) | 5 (16.1%) | 2 (6.5%) | 1.0 | 1.39 | A-D | 0.154 |
| E) MS 60 mg/NTX 0.01 mg | 23 | 12 (52.2%) | 1 (4.3%) | 2 (8.7%) | 4 (17.4%) | 4 (17.4%) | 1.4 | 1.67 | A-E | 0.035* |
| F) MS 60 mg/NTX 0.1 mg | 22 | 8(36.4%) | 3 (13.6%) | 2 (9.1%) | 5 (22.7%) | 4 (18.2%) | 1.7 | 1.61 | A-F | 0.004** |
| | | | | | | | | | B-C | 0.413 |
| | | | | | | | | | B-D | 0.085 |
| | | | | | | | | | B-E | 0.012* |
| | | | | | | | | | B-F | 0.001** |
| | | | | | | | | | C-D | 0.040* |
| | | | | | | | | | C-E | 0.008** |
| | | | | | | | | | C-F | <0.001*** |
| | | | | | | | | | D-E | 0.292 |
| | | | | | | | | | D-F | 0.060 |
| | | | | | | | | | E-F | 0.510 |

[1] FROM COCHRAN-MANTEL-HAENZEL TEST FOR RAW MEAN SCORES DIFFERENCE, ADJUSTING FOR SITE.
*, **, ***P-VALUE <=0.05, <=0.01, OR <=0.001 RESPECTIVELY.

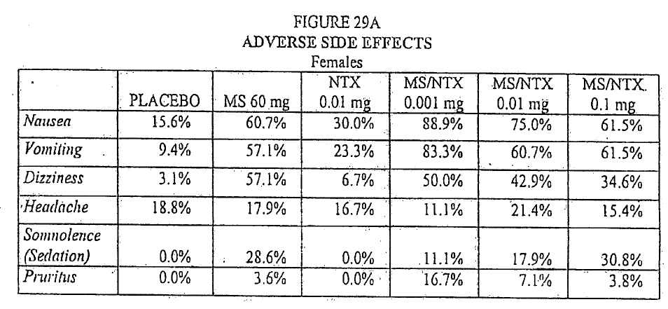

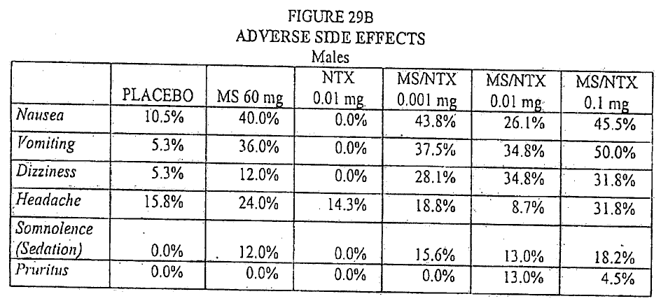

The majority of adverse events reported were categorized as digestive (nausea or vomiting) or nervous system (dizziness or somnolence) as further shown in Tables 52A and 52B for females and Tables 52C and 52D for males. FIGS. 29A for females and 29B for males represent a summary of exemplary adverse side effects according to methods and compositions of the invention. In females, the placebo group has the lowest incidence of adverse events for nausea, vomiting, and dizziness. For somnolence (sedation), both the placebo group and the NTX alone group have the lowest incidence. In males, the NTX alone group has the lowest incidence of nausea, vomiting and dizziness. For somnolence (sedation), the placebo group and the NTX alone group have the lowest incidence.

TABLE 52A
Adverse Events By Body System And Severity
Intent-To-Treat Population, Female Patients

| | | Total | | | | | | | |
|---|---|---|---|---|---|---|---|---|---|
| | | No. | | | | No. | | | |
| Body System | | Of | No. Of | | | Of | | | |
| Adverse Events | | Pa- | Patients | | | E- | Severity [2] | | |
| (Costart English) | Treatment | tients | W/Event | Source | P-Value [1] | vents | Mild | Moderate | Severe |

TOTAL NUMBER OF EVENTS
ADVERSE EVENTS
(ALL BODY SYSTEMS)

| | | | | | | | | | |
|---|---|---|---|---|---|---|---|---|---|
| All EVENTS | A) PLACEBO | 32 | 16 (50.0%) | Treatment | <0.001*** | 27 | 8 (29.6%) | 7 (25.9%) | 12 (44.4%) |
| | B) MS 60 mg | 28 | 26 (92.9%) | A-B | <0.001*** | 116 | 32 (27.6%) | 55 (47.4%) | 29 (25.0%) |
| | C) NTX 0.01 mg | 30 | 21 (70.0%) | A-D | <0.001*** | 48 | 12 (25.0%) | 21 (43.8%) | 15 (31.3%) |
| | D) MS 60 mg/NTX 0.001 mg | 18 | 18 (100.0%) | A-E | <0.001*** | 66 | 15 (22.7%) | 29 (43.9%) | 22 (33.3%) |
| | E) MS 60 mg/NTX 0.01 mg | 28 | 28 (100.0%) | A-F | <0.001*** | 103 | 33 (32.0%) | 38 (36.9%) | 32 (31.1%) |
| | F) MS 60 mg/NTX 0.1 mg | 26 | 24 (92.3%) | B-C | 0.026* | 86 | 22 (25.6%) | 40 (46.5%) | 24 (27.9%) |
| | | | | C-D | 0.009** | | | | |
| | | | | C-E | 0.001** | | | | |
| | | | | C-F | 0.036* | | | | |

CARDIAC DISORDERS

| | | | | | | | | | |
|---|---|---|---|---|---|---|---|---|---|
| ALL EVENTS | A) PLACEBO | 32 | 0 | Treatment | 0.328 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 1 (3.3%) | | | 1 | 0 | 1 (100.0%) | 0 |
| | D) MS 60 mg/NTX 0.001 mg | 18 | 2 (11.1%) | | | 2 | 1 (50.0%) | 1 (50.0%) | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 0 | | | 0 | 0 | 0 | 0 |
| PALPITATIONS | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 0 | | | 0 | 0 | 0 | 0 |
| TACHYCARDIA | A) PLACEBO | 32 | 0 | Treatment | 0.156 | 0 | 0 | 0 | 0 |
| NOS | B) MS 60 mg | 28 | 1 (3.6%) | | | 1 | 1 (100.0%) | 0 | 0 |
| | C) NTX 0.01 mg | 30 | 1 (3.3%) | | | 1 | 0 | 1 (100.0%) | 0 |
| | D) MS 60 mg/NTX 0.001 mg | 18 | 2 (11.1%) | | | 2 | 1 (50.0%) | 1 (50.0%) | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

EAR AND LABYRINTH DISORDERS

| | | | | | | | | | |
|---|---|---|---|---|---|---|---|---|---|
| ALL EVENTS | A) PLACEBO | 32 | 2 (6.3%) | Treatment | 0.454 | 3 | 2 (66.7%) | 1 (33.3%) | 0 |
| | B) MS 60 mg | 28 | 1 (3.6%) | | | 1 | 1 (100.0%) | 0 | 0 |
| | C) NTX 0.01 mg | 30 | 2 (6.7%) | | | 2 | 0 | 2 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 3 (10.7%) | | | 3 | 0 | 3 (100.0%) | 0 |
| | F) MS 60 mg/NTX 0.1 mg | 26 | 0 | | | 0 | 0 | 0 | 0 |
| EARACHE | A) PLACEBO | 32 | 2 (6.3%) | Treatment | 0.413 | 3 | 2 (66.7%) | 1 (33.3%) | 0 |
| | B) MS 60 mg | 28 | 0 | | | 0 | 0 | 0 | 0 |
| | C) NTX 0.01 mg | 30 | 2 (6.7%) | | | 2 | 0 | 2 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 2 (7.1%) | | | 2 | 0 | 2 (100.0%) | 0 |
| | F) MS 60 mg/NTX 0.1 mg | 26 | 0 | | | 0 | 0 | 0 | 0 |
| HEARING | A) PLACEBO | 32 | 0 | Treatment | 0.438 | 0 | 0 | 0 | 0 |
| IMPAIRED | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 1 (100.0%) | 0 |
| | F) MS 60 mg/NTX 0.1 mg | 26 | 0 | | | 0 | 0 | 0 | 0 |
| HYPERACUSIS | A) PLACEBO | 32 | 0 | Treatment | 0.438 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

EYE DISORDERS

| | | | | | | | | | |
|---|---|---|---|---|---|---|---|---|---|
| ALL EVENTS | A) PLACEBO | 32 | 0 | Treatment | 0.008** | 0 | 0 | 0 | 0 |
| | B) MS 60 mg | 28 | 6 (21.4%) | A-B | 0.005** | 6 | 3 (50.0%) | 2 (33.3%) | 1 (16.7%) |
| | C) NTX 0.01 mg | 30 | 0 | A-F | 0.048* | 0 | 0 | 0 | 0 |
| | D) MS 60 mg/NTX 0.001 mg | 18 | 1 (5.6%) | B-C | 0.007** | 1 | 1 (100.0%) | 0 | 0 |
| | E) MS 60 mg/NTX 0.01 mg | 28 | 1 (3.6%) | B-E | 0.043* | 1 | 1 (100.0%) | 0 | 0 |
| | F) MS 60 mg/NTX 0.1 mg | 26 | 3 (11.5%) | | | 3 | 3 (100.0%) | 0 | 0 |
| AMBLYOPIA NOS | A) PLACEBO | 32 | 0 | Treatment | 0.384 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 1 (100.0%) | 0 | 0 |
| CONJUNCTIVITIS | A) PLACEBO | 32 | 0 | Treatment | 0.109 | 0 | 0 | 0 | 0 |
| NEC | B) MS 60 mg | 28 | 4 (14.3%) | A-B | 0.026* | 4 | 3 (75.0%) | 1 (25.0%) | 0 |
| | C) NTX 0.01 mg | 30 | 0 | B-C | 0.031* | 0 | 0 | 0 | 0 |
| | D) MS 60 mg/NTX 0.001 mg | 18 | 1 (5.6%) | | | 1 | 1 (100.0%) | 0 | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 2 (7.7%) | | | 2 | 2 (100.0%) | 0 | 0 |
| RED EYE | A) PLACEBO | 32 | 0 | Treatment | 0.438 | 0 | 0 | 0 | 0 |
| | B) MS 60 mg | 28 | 1 (3.6%) | | | 1 | 0 | 0 | 1 (100.0%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| VISION BLURRED | A) PLACEBO | 32 | 0 | Treatment | 0.438 | 0 | 0 | 0 | 0 |
| | B) MS 60 mg | 28 | 1 (3.6%) | | | 1 | 0 | 1 (100.0%) | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

GASTROINTESTINAL DISORDERS

| | | | | | | | | | |
|---|---|---|---|---|---|---|---|---|---|
| ALL EVENTS | A) PLACEBO | 32 | 9 (28.1%) | Treatment | <0.001*** | 11 | 3 (27.3%) | 3 (27.3%) | 5 (45.5%) |
| | B) MS 60 mg | 28 | 22 (78.6%) | A-B | <0.001*** | 40 | 6 (15.0%) | 17 (42.5%) | 17 (42.5%) |
| | C) NTX 0.01 mg | 30 | 13 (43.3%) | A-D | <0.001*** | 19 | 6 (31.6%) | 6 (31.6%) | 7 (36.8%) |
| | D) MS 60 mg/NTX 0.001 mg | 18 | 17 (94.4%) | A-E | <0.001*** | 35 | 5 (14.3%) | 13 (37.1%) | 17 (48.6%) |
| | E) MS 60 mg/NTX 0.01 mg | 28 | 24 (85.7%) | A-F | <0.001*** | 44 | 10 (22.7%) | 13 (29.5%) | 21 (47.7%) |
| | F) MS 60 mg/NTX 0.1 mg | 26 | 20 (76.9%) | B-C | 0.006** | 40 | 3 (7.5%) | 20 (50.0%) | 17 (42.5%) |
| | | | | C-D | <0.001*** | | | | |
| | | | | C-E | <0.001*** | | | | |
| | | | | C-F | 0.010* | | | | |
| ABDOMINAL | A) PLACEBO | 32 | 0 | Treatment | 0.438 | 0 | 0 | 0 | 0 |
| PAIN UPPER | B) MS 60 mg | 28 | 1 (3.6%) | | | 1 | 0 | 0 | 1 (100.0%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| DYSPEPSIA | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 1 (100.0%) | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| DYSPHAGIA | A) PLACEBO | 32 | 0 | Treatment | 0.153 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 0 | 1 (100.0%) | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| MELAENA | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 1 (100.0%) | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| NAUSEA | A) PLACEBO | 32 | 5 (15.6%) | Treatment | <0.001*** | 6 | 2 (33.3%) | 1 (16.7%) | 3 (50.0%) |
| | B) MS 60 mg | 28 | 17 (60.7%) | A-B | <0.001*** | 21 | 5 (23.8%) | 12 (57.1%) | 4 (19.0%) |
| | C) NTX 0.01 mg | 30 | 9 (30.0%) | A-D | <0.001*** | 10 | 3 (30.0%) | 5 (50.0%) | 2 (20.0%) |
| | D) MS 60 mg/NTX 0.001 mg | 18 | 16 (88.9%) | A-E | <0.001*** | 16 | 4 (25.0%) | 9 (56.3%) | 3 (18.8%) |
| | E) MS 60 mg/NTX 0.01 mg | 28 | 21 (75.0%) | A-F | <0.001*** | 25 | 7 (28.0%) | 10 (40.0%) | 8 (32.0%) |
| | F) MS 60 mg/NTX 0.1 mg | 26 | 16 (61.5%) | B-C | 0.018* | 18 | 1 (5.6%) | 15 (83.3%) | 2 (11.1%) |
| | | | | B-D | 0.038* | | | | |
| | | | | C-D | <0.001*** | | | | |
| | | | | C-E | <0.001*** | | | | |
| | | | | C-F | 0.017* | | | | |
| | | | | D-F | 0.045* | | | | |
| ORAL PAIN | A) PLACEBO | 32 | 0 | Treatment | 0.048* | 0 | 0 | 0 | 0 |
| | B) MS 60 mg | 28 | 1 (3.6%) | | | 1 | 0 | 0 | 1 (100.0%) |
| | C) NTX 0.01 mg | 30 | 0 | | | 0 | 0 | 0 | 0 |
| | D) MS 60 mg/NTX 0.001 mg | 18 | 2 (11.1%) | | | 2 | 0 | 0 | 2 (100.0%) |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| SORE THROAT | A) PLACEBO | 32 | 2 (6.3%) | Treatment | 0.144 | 2 | 0 | 2 (100.0%) | 0 |
| NOS | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| STOMATITIS | A) PLACEBO | 32 | 0 | Treatment | 0.541 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 1 (3.8%) | | | 1 | 0 | 0 | 1 (100.0%) |
| VOMITING NOS | A) PLACEBO | 32 | 3 (9.4%) | Treatment | <0.001*** | 3 | 1 (33.3%) | 0 | 2 (66.7%) |
| | B) MS 60 mg | 28 | 16 (57.1%) | A-B | <0.001*** | 17 | 1 (5.9%) | 5 (29.4%) | 11 (64.7%) |
| | C) NTX 0.01 mg | 30 | 7 (23.3%) | A-D | <0.001*** | 7 | 1 (14.3%) | 1 (14.3%) | 5 (71.4%) |
| | D) MS 60 mg/NTX 0.001 mg | 18 | 15 (83.3%) | A-E | <0.001*** | 16 | 1 (6.3%) | 3 (18.8%) | 12 (75.0%) |
| | E) MS 60 mg/NTX 0.01 mg | 28 | 17 (60.7%) | A-F | <0.001*** | 18 | 3 (16.7%) | 3 (16.7%) | 12 (66.7%) |
| | F) MS 60 mg/NTX 0.1 mg | 26 | 16 (61.5%) | B-C | 0.008** | 21 | 2 (9.5%) | 5 (23.8%) | 14 (66.7%) |
| | | | | C-D | <0.001*** | | | | |
| | | | | C-E | 0.003** | | | | |
| | | | | C-F | 0.003** | | | | |

GENERAL DISORDERS AND ADMINISTRATION SITE CONDITIONS

| | | | | | | | | | |
|---|---|---|---|---|---|---|---|---|---|
| ALL EVENTS | A) PLACEBO | 32 | 2 (6.3%) | Treatment | 0.214 | 2 | 1 (50.0%) | 0 | 1 (50.0%) |
| | B) MS 60 mg | 28 | 8 (28.6%) | A-B | 0.020* | 8 | 3 (37.5%) | 5 (62.5%) | 0 |
| | C) NTX 0.01 mg | 30 | 3 (10.0%) | | | 3 | 1 (33.3%) | 1 (33.3%) | 1 (33.3%) |
| | D) MS 60 mg/NTX 0.001 mg | 18 | 3 (16.7%) | | | 3 | 1 (33.3%) | 2 (66.7%) | 0 |
| | E) MS 60 mg/NTX 0.01 mg | 28 | 5 (17.9%) | | | 8 | 4 (50.0%) | 2 (25.0%) | 2 (25.0%) |
| | F) MS 60 mg/NTX 0.1 mg | 26 | 3 (11.5%) | | | 3 | 2 (66.7%) | 1 (33.3%) | 0 |
| ASTHENIA | A) PLACEBO | 32 | 0 | Treatment | 0.124 | 0 | 0 | 0 | 0 |
| | B) MS 60 mg | 28 | 3 (10.7%) | | | 3 | 2 (66.7%) | 1 (33.3%) | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 0 | 1 (100.0%) | 0 |
| | E) MS 60 mg/NTX 0.01 mg | 28 | 1 (3.6%) | | | 2 | 1 (50.0%) | 0 | 1 (50.0%) |
| | F) MS 60 mg/NTX 0.1 mg | 26 | 0 | | | 0 | 0 | 0 | 0 |
| FATIGUE | A) PLACEBO | 32 | 0 | Treatment | 0.438 | 0 | 0 | 0 | 0 |
| | B) MS 60 mg | 28 | 1 (3.6%) | | | 1 | 0 | 1 (100.0%) | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| FEELING JITTERY | A) PLACEBO | 32 | 0 | Treatment | 0.298 | 0 | 0 | 0 | 0 |
| | B) MS 60 mg | 28 | 2 (7.1%) | | | 2 | 1 (50.0%) | 1 (50.0%) | 0 |
| | C) NTX 0.01 mg | 30 | 0 | | | 0 | 0 | 0 | 0 |
| | D) MS 60 mg/NTX 0.001 mg | 18 | 2 (11.1%) | | | 2 | 1 (50.0%) | 1 (50.0%) | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 1 (3.8%) | | | 1 | 0 | 1 (100.0%) | 0 |
| PAIN IN FACE | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 0 | | | 0 | 0 | 0 | 0 |
| PAIN NOS | A) PLACEBO | 32 | 1 (3.1%) | Treatment | 0.782 | 1 | 0 | 0 | 1 (100.0%) |
| | B) MS 60 mg | 28 | 1 (3.6%) | | | 1 | 0 | 1 (100.0%) | 0 |
| | C) NTX 0.01 mg | 30 | 1 (3.3%) | | | 1 | 0 | 0 | 1 (1.00.0%) |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| PYREXIA | A) PLACEBO | 32 | 1 (3.1%) | Treatment | 0.893 | 1 | 1 (100.0%) | 0 | 0 |
| | B) MS 60 mg | 28 | 1 (3.6%) | | | 1 | 0 | 1 (100.0%) | 0 |
| | C) NTX 0.01 mg | 30 | 1 (3.3%) | | | 1 | 1 (100.0%) | 0 | 0 |
| | D) MS 60 mg/NTX 0.001 mg | 18 | 0 | | | 0 | 0 | 0 | 0 |
| | E) MS 60 mg/NTX 0.01 mg | 28 | 2 (7.1%) | | | 2 | 1 (50.0%) | 1 (50.0%) | 0 |
| | F) MS 60 mg/NTX 0.1 mg | 26 | 1 (3.8%) | | | 1 | 1 (100.0%) | 0 | 0 |
| RIGORS | A) PLACEBO | 32 | 0 | Treatment | 0.384 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 1 (100.0%) | 0 | 0 |
| SHIVERING | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 0 | 1 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| WEAKNESS | A) PLACEBO | 32 | 0 | Treatment | 0.084 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 2 (7.1%) | | | 2 | 1 (50.0%) | 1 (50.0%) | 0 |
| | F) MS 60 mg/NTX 0.1 mg | 26 | 0 | | | 0 | 0 | 0 | 0 |

HEPATO-BILIARY DISORDERS

| | | | | | | | | | |
|---|---|---|---|---|---|---|---|---|---|
| ALL EVENTS | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0) |
| | F) MS 60 mg/NTX 0.1 mg | 26 | 0 | | | 0 | 0 | 0 | 0 |
| CHOLELITHIASIS | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 0 | | | 0 | 0 | 0 | 0 |

INFECTIONS AND INFESTATIONS

| | | | | | | | | | |
|---|---|---|---|---|---|---|---|---|---|
| ALL EVENTS | A) PLACEBO | 32 | 4 (12.5%) | Treatment | 0.400 | 4 | 0 | 0 | 4 (100.0%) |
| | B) MS 60 mg | 28 | 4 (14.3%) | | | 5 | 1 (20.0%) | 3 (60.0%) | 1 (20.0%) |
| | C) NTX 0.01 mg | 30 | 7 (23.3%) | | | 8 | 1 (12.5%) | 3 (37.5%) | 4 (50.0%) |
| | D) MS 60 mg/NTX 0.001 mg | 18 | 4 (22.2%) | | | 4 | 0 | 1 (25.0%) | 3 (75.0%) |
| | E) MS 60 mg/NTX 0.01 mg | 28 | 2 (7.1%) | | | 2 | 0 | 0 | 2 (100.0%) |
| | F) MS 60 mg/NTX 0.1 mg | 26 | 2 (7.7%) | | | 3 | 0 | 1 (33.3%) | 2 (66.7%) |
| CELLULITIS | A) PLACEBO | 32 | 0 | Treatment | 0.112 | 0 | 0 | 0 | 0 |
| | B) MS 60 mg | 28 | 0 | | | 0 | 0 | 0 | 0 |
| | C) NTX 0.01 mg | 30 | 2 (6.7%) | | | 2 | 0 | 0 | 2 (100.0%) |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| DRY SOCKET | A) PLACEBO | 32 | 2 (6.3%) | Treatment | 0.868 | 2 | 0 | 0 | 2 (100.0%) |
| NOS | B) MS 60 mg | 28 | 2 (7.1%) | | | 2 | 0 | 1 (50.0%) | 1 (50.0%) |
| | C) NTX 0.01 mg | 30 | 3 (10.0%) | | | 3 | 0 | 2 (66.7%) | 1 (33.3%) |
| | D) MS 60 mg/NTX 0.001 mg | 18 | 2 (11.1%) | | | 2 | 0 | 0 | 2 (100.0%) |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 1 (3.8%) | | | 2 | 0 | 0 | 2 (100.0%) |
| ORAL INFECTION | A) PLACEBO | 32 | 0 | Treatment | 0.153 | 0 | 0 | 0 | 0 |
| NEC | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 0 | 1 (100.0%) | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| PHARYNGITIS | A) PLACEBO | 32 | 2 (6.3%) | Treatment | 0.988 | 2 | 0 | 0 | 2 (100.0%) |
| NOS | B) MS 60 mg | 28 | 2 (7.1%) | | | 3 | 1 (33.3%) | 2 (66.7%) | 0 |
| | C) NTX 0.01 mg | 30 | 2 (6.7%) | | | 3 | 1 (33.3%) | 1 (33.3%) | 1 (33.3%) |
| | D) MS 60 mg/NTX 0.001 mg | 18 | 1 (5.6%) | | | 1 | 0 | 0 | 1 (100.0%) |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 1 (3.8%) | | | 1 | 0 | 1 (100.0%) | 0 |

MUSCULOSKELETAL, CONNECTIVE TISSUE AND BONE DISORDERS

| | | | | | | | | | |
|---|---|---|---|---|---|---|---|---|---|
| ALL EVENTS | A) PLACEBO | 32 | 0 | Treatment | 0.238 | 0 | 0 | 0 | 0 |
| | B) MS 60 mg | 28 | 1 (3.6%) | | | 3 | 0 | 2 (66.7%) | 1 (33.3%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 2 (7.1%) | | | 2 | 1 (50.0%) | 1 (50.0%) | 0 |
| | F) MS 60 mg/NTX 0.1 mg | 26 | 0 | | | 0 | 0 | 0 | 0 |
| JOINT DISORDER | A) PLACEBO | 32 | 0 | Treatment | 0.438 | 0 | 0 | 0 | 0 |
| NOS | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 1 (100.0%) | 0 |
| | F) MS 60 mg/NTX 0.1 mg | 26 | 0 | | | 0 | 0 | 0 | 0 |
| MUSCLE | A) PLACEBO | 32 | 0 | Treatment | 0.438 | 0 | 0 | 0 | 0 |
| TWITCHING | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 0 | | | 0 | 0 | 0 | 0 |
| MYALGIA | A) PLACEBO | 32 | 0 | Treatment | 0.438 | 0 | 0 | 0 | 0 |
| | B) MS 60 mg | 28 | 1 (3.6%) | | | 1 | 0 | 1 (100.0%) | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| SENSATION OF | A) PLACEBO | 32 | 0 | Treatment | 0.438 | 0 | 0 | 0 | 0 |
| HEAVINESS | B) MS 60 mg | 28 | 1 (3.6%) | | | 2 | 0 | 1 (50.0%) | 1 (50.0%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

NEOPLASMS BENIGN AND MALIGNANT (INCLUDING CYSTS AND POLYPS)

| | | | | | | | | | |
|---|---|---|---|---|---|---|---|---|---|
| ALL EVENTS | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 0 | 0 | 1 (100.0%) |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| ADENOMA | A) PLACEBO | 32 | 0 | Treatment | 0.489 | 0 | 0 | 0 | 0 |
| BENIGN NOS | B) MS 60 mg | 28 | 0 | | | 0 | 0 | 0 | 0 |
| | C) NTX 0.01 mg | 30 | 1 (3.3%) | | | 1 | 0 | 0 | 1 (100.0%) |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

NERVOUS SYSTEM DISORDERS

| | | | | | | | | | |
|---|---|---|---|---|---|---|---|---|---|
| ALL EVENTS | A) PLACEBO | 32 | 7 (21.9%) | Treatment | <0.001*** | 7 | 2 (28.6%) | 3 (42.9%) | 2 (28.6%) |
| | B) MS 60 mg | 28 | 20 (71.4%) | A-B | <0.001*** | 37 | 7 (18.9%) | 24 (64.9%) | 6 (16.2%) |
| | C) NTX 0.01 mg | 30 | 10 (33.3%) | A-D | 0.005** | 11 | 3 (27.3%) | 7 (63.6%) | 1 (9.1%) |
| | D) MS 60 mg/NTX 0.001 mg | 18 | 11 (61.1%) | A-E | <0.001*** | 14 | 4 (28.6%) | 9 (64.3%) | 1 (7.1%) |
| | E) MS 60 mg/NTX 0.01 mg | 28 | 19 (67.9%) | A-F | 0.005** | 29 | 10 (34.5%) | 16 (55.2%) | 3 (10.3%) |
| | F) MS 60 mg/NTX 0.1 mg | 26 | 15 (57.7%) | B-C | 0.003** | 24 | 10 (41.7%) | 10 (41.7%) | 4 (16.7%) |
| | | | | C-E | 0.008** | | | | |
| DIZZINESS | A) PLACEBO | 32 | 1 (3.1%) | Treatment | <0.001*** | 1 | 0 | 1 (100.0%) | 0 |
| (EXC VERTIGO) | B) MS 60 mg | 28 | 16 (57.1%) | A-B | <0.001*** | 18 | 3 (16.7%) | 12 (66.7%) | 3 (16.7%) |
| | C) NTX 0.01 mg | 30 | 2 (6.7%) | A-D | <0.001*** | 2 | 2 (100.0%) | 0 | 0 |
| | D) MS 60 mg/NTX 0.001 mg | 18 | 9 (50.0%) | A-E | <0.001*** | 9 | 3 (33.3%) | 6 (66.7%) | 0 |
| | E) MS 60 mg/NTX 0.01 mg | 28 | 12 (42.9%) | A-F | 0.001** | 14 | 5 (35.7%) | 8 (57.1%) | 1 (7.1%) |
| | F) MS 60 mg/NTX 0.1 mg | 26 | 9 (34.6%) | B-C | <0.001*** | 10 | 3 (30.0%) | 5 (50.0%) | 2 (20.0%) |
| | | | | C-D | <0.001*** | | | | |
| | | | | C-E | 0.001** | | | | |
| | | | | C-F | 0.008** | | | | |
| HEADACHE NOS | A) PLACEBO | 32 | 6 (18.8%) | Treatment | 0.966 | 6 | 2 (33.3%) | 2 (33.3%) | 2 (33.3%) |
| | B) MS 60 mg | 28 | 5 (17.9%) | | | 5 | 1 (20.0%) | 4 (80.0%) | 0 |
| | C) NTX 0.01 mg | 30 | 5 (16.7%) | | | 5 | 1 (20.0%) | 3 (60.0%) | 1 (20.0%) |
| | D) MS 60 mg/NTX 0.001 mg | 18 | 2 (11.1%) | | | 2 | 0 | 1 (50.0%) | 1 (50.0%) |
| | E) MS 60 mg/NTX 0.01 mg | 28 | 6 (21.4%) | | | 6 | 1 (16.7%) | 4 (66.7%) | 1 (16.7%) |
| | F) MS 60 mg/NTX 0.1 mg | 26 | 4 (15.4%) | | | 4 | 1 (25.0%) | 2 (50.0%) | 1 (25.0%) |
| HYPERTONIA | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 0 | 1 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| HYPOTONIA | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 1 (100.0%) | 0 |
| | F) MS 60 mg/NTX 0.1 mg | 26 | 0 | | | 0 | 0 | 0 | 0 |
| PARAESTHESIA | A) PLACEBO | 32 | 0 | Treatment | 0.657 | 0 | 0 | 0 | 0 |
| NEC | B) MS 60 mg | 28 | 3 (10.7%) | | | 5 | 2 (40.0%) | 2 (40.0%) | 1 (20.0%) |
| | C) NTX 0.01 mg | 30 | 2 (6.7%) | | | 2 | 0 | 2 (100.0%) | 0 |
| | D) MS 60 mg/NTX 0.001 mg | 18 | 1 (5.6%) | | | 1 | 1 (100.0%) | 0 | 0 |
| | E) MS 60 mg/NTX 0.01 mg | 28 | 2 (7.1%) | | | 2 | 1 (50.0%) | 1 (50.0%) | 0 |
| | F) MS 60 mg/NTX 0.1 mg | 26 | 2 (7.7%) | | | 2 | 1 (50.0%) | 1 (50.0%) | 0 |
| SOMNOLENCE | A) PLACEBO | 32 | 0 | Treatment | <0.001*** | 0 | 0 | 0 | 0 |
| | B) MS 60 mg | 28 | 8 (28.6%) | A-B | 0.001** | 9 | 1 (11.1%) | 6 (66.7%) | 2 (22.2%) |
| | C) NTX 0.01 mg | 30 | 0 | A-E | 0.012* | 0 | 0 | 0 | 0 |
| | D) MS 60 mg/NTX 0.001 mg | 18 | 2 (11.1%) | A-F | <0.001*** | 2 | 0 | 2 (100.0%) | 0 |
| | E) MS 60 mg/NTX 0.01 mg | 28 | 5 (17.9%) | B-C | 0.001** | 5 | 3 (60.0%) | 2 (40.0%) | 0 |
| | F) MS 60 mg/NTX 0.1 mg | 26 | 8 (30.8%) | C-E | 0.015* | 8 | 5 (62.5%) | 2 (25.0%) | 1 (12.5%) |
| | | | | C-F | 0.001** | | | | |
| TASTE LOSS | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 0 | 1 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| TREMOR NEC | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 0 | | | 0 | 0 | 0 | 0 |

PREGNANCY, PUERPERIUM AND PERINATAL CONDITIONS

| | | | | | | | | | |
|---|---|---|---|---|---|---|---|---|---|
| ALL EVENTS | A) PLACEBO | 32 | 0 | Treatment | 0.438 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| PREGNANCY NOS | A) PLACEBO | 32 | 0 | Treatment | 0.438 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

PSYCHIATRIC DISORDERS

| | | | | | | | | | |
|---|---|---|---|---|---|---|---|---|---|
| ALL EVENTS | A) PLACEBO | 32 | 0 | Treatment | 0.156 | 0 | 0 | 0 | 0 |
| | B) MS 60 mg | 28 | 4 (14.3%) | A-B | 0.026* | 5 | 1 (20.0%) | 1 (20.0%) | 3 (60.0%) |
| | C) NTX 0.01 mg | 30 | 1 (3.3%) | | | 1 | 0 | 1 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 3 (10.7%) | | | 3 | 3 (100.0%) | 0 | 0 |
| | F) MS 60 mg/NTX 0.1 mg | 26 | 2 (7.7%) | | | 4 | 1 (25.0%) | 3 (75.0%) | 0 |
| ANXIETY NEC | A) PLACEBO | 32 | 0 | Treatment | 0.438 | 0 | 0 | 0 | 0 |
| | B) MS 60 mg | 28 | 1 (3.6%) | | | 1 | 0 | 0 | 1 (100.0%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| CONFUSION | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 0 | | | 0 | 0 | 0 | 0 |
| DEPERSONALI- | A) PLACEBO | 32 | 0 | Treatment | 0.541 | 0 | 0 | 0 | 0 |
| SATION | B) MS 60 mg | 28 | 1 (3.6%) | | | 1 | 0 | 0 | 1 (100.0%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 1 (100.0%) | 0 | 0 |
| DISSOCIATION | A) PLACEBO | 32 | 0 | Treatment | 0.384 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 0 | 1 (100.0%) | 0 |
| EUPHORIC MOOD | A) PLACEBO | 32 | 0 | Treatment | 0.541 | 0 | 0 | 0 | 0 |
| | B) MS 60 mg | 28 | 1 (3.6%) | | | 1 | 0 | 0 | 1 (100.0%) |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 0 | 1 (100.0%) | 0 |
| NERVOUSNESS | A) PLACEBO | 32 | 0 | Treatment | 0.579 | 0 | 0 | 0 | 0 |
| | B) MS 60 mg | 28 | 2 (7.1%) | | | 2 | 1 (50.0%) | 1 (50.0%) | 0 |
| | C) NTX 0.01 mg | 30 | 1 (3.3%) | | | 1 | 0 | 1 (100.0%) | 0 |
| | D) MS 60 mg/NTX 0.001 mg | 18 | 0 | | | 0 | 0 | 0 | 0 |
| | E) MS 60 mg/NTX 0.01 mg | 28 | 2 (7.1%) | | | 2 | 2 (100.0%) | 0 | 0 |
| | F) MS 60 mg/NTX 0.1 mg | 26 | 1 (3.8%) | | | 1 | 0 | 1 (100.0%) | 0 |

RENAL AND URINARY DISORDERS

| | | | | | | | | | |
|---|---|---|---|---|---|---|---|---|---|
| ALL EVENTS | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 1 (100.0%) | 0 |
| | F) MS 60 mg/NTX 0.1 mg | 26 | 0 | | | 0 | 0 | 0 | 0 |
| URINARY | A) PLACEBO | 32 | 0 | Treatment | 0.438 | 0 | 0 | 0 | 0 |
| RETENTION | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 1 (100.0%) | 0 |
| | F) MS 60 mg/NTX 0.1 mg | 26 | 0 | | | 0 | 0 | 0 | 0 |

REPRODUCTIVE SYSTEM AND BREAST DISORDERS

| | | | | | | | | | |
|---|---|---|---|---|---|---|---|---|---|
| ALL EVENTS | A) PLACEBO | 32 | 0 | Treatment | 0.153 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 0 | 0 | 1 (100.0%) |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| DYSMEN- | A) PLACEBO | 32 | 0 | Treatment | 0.153 | 0 | 0 | 0 | 0 |
| ORRHOEA | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 0 | 0 | 1 (100.0%) |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |

RESPIRATORY, THORACIC AND MEDIASTINAL DISORDERS

| | | | | | | | | | |
|---|---|---|---|---|---|---|---|---|---|
| ALL EVENTS | A) PLACEBO | 32 | 0 | Treatment | 0.768 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 1 (3.3%) | | | 1 | 0 | 0 | 1 (100.0%) |
| | D) MS 60 mg/NTX 0.001 mg | 18 | 1 (5.6%) | | | 1 | 1 (100.0%) | 0 | 0 |
| | E) MS 60 mg/NTX 0.01 mg | 28 | 1 (3.6%) | | | 2 | 0 | 0 | 2 (100.0%) |
| | F) MS 60 mg/NTX 0.1 mg | 26 | 0 | | | 0 | 0 | 0 | 0 |
| COUGH | A) PLACEBO | 32 | 0 | Treatment | 0.489 | 0 | 0 | 0 | 0 |
| | B) MS 60 mg | 28 | 0 | | | 0 | 0 | 0 | 0 |
| | C) NTX 0.01 mg | 30 | 1 (3.3%) | | | 1 | 0 | 0 | 1 (100.0%) |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| EPISTAXIS | A) PLACEBO | 32 | 0 | Treatment | 0.153 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 1 (100.0%) | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| RHINITIS NOS | A) PLACEBO | 32 | 0 | Treatment | 0.573 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 0 | | | 0 | 0 | 0 | 0 |
| SINUS | A) PLACEBO | 32 | 0 | Treatment | 0.438 | 0 | 0 | 0 | 0 |
| CONGESTION | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 0 | 0 | 1 (100.0%) |
| | F) MS 60 mg/NTX 0.1 mg | 26 | 0 | | | 0 | 0 | 0 | 0 |

SKIN & SUBCUTANEOUS TISSUE DISORDERS

| | | | | | | | | | |
|---|---|---|---|---|---|---|---|---|---|
| ALL EVENTS | A) PLACEBO | 32 | 0 | Treatment | 0.087 | 0 | 0 | 0 | 0 |
| | B) MS 60 mg | 28 | 2 (7.1%) | A-D | 0.017* | 4 | 3 (75.0%) | 1 (25.0%) | 0 |
| | C) NTX 0.01 mg | 30 | 0 | C-D | 0.020* | 0 | 0 | 0 | 0 |
| | D) MS 60 mg/NTX 0.001 mg | 18 | 3 (16.7%) | | | 5 | 2 (40.0%) | 3 (60.0%) | 0 |
| | E) MS 60 mg/NTX 0.01 mg | 28 | 3 (10.7%) | | | 3 | 2 (66.7%) | 0 | 1 (33.3%) |
| | F) MS 60 mg/NTX 0.1 mg | 26 | 1 (3.8%) | | | 2 | 0 | 1 (50.0%) | 1 (50.0%) |
| DERMATITIS NOS | A) PLACEBO | 32 | 0 | Treatment | 0.573 | 0 | 0 | 0 | 0 |
| | B) MS 60 mg | 28 | 1 (3.6%) | | | 1 | 1 (100.0%) | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 0 | | | 0 | 0 | 0 | 0 |
| ECCHYMOSIS | A) PLACEBO | 32 | 0 | Treatment | 0.153 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 1 (5.6%) | | | 1 | 1 (100.0%) | 0 | 0 |
| | E) MS 60 mg/NTX 0.01 mg | 28 | 0 | | | 0 | 0 | 0 | 0 |
| | F) MS 60 mg/NTX 0.1 mg | 26 | 0 | | | 0 | 0 | 0 | 0 |
| PRURITUS NOS | A) PLACEBO | 32 | 0 | Treatment | 0.074 | 0 | 0 | 0 | 0 |
| | B) MS 60 mg | 28 | 1 (3.6%) | A-D | 0.017* | 1 | 0 | 1 (100.0%) | 0 |
| | C) NTX 0.01 mg | 30 | 0 | C-D | 0.020* | 0 | 0 | 0 | 0 |
| | D) MS 60 mg/NTX 0.001 mg | 18 | 3 (16.7%) | | | 4 | 1 (25.0%) | 3 (75.0%) | 0 |
| | E) MS 60 mg/NTX 0.01 mg | 28 | 2 (7.1%) | | | 2 | 1 (50.0%) | 0 | 1 (50.0%) |
| | F) MS 60 mg/NTX 0.1 mg | 26 | 1 (3.8%) | | | 1 | 0 | 0 | 1 (100.0%) |
| URTICARIA NOS | A) PLACEBO | 32 | 0 | Treatment | 0.541 | 0 | 0 | 0 | 0 |
| | B) MS 60 mg | 28 | 1 (3.6%) | | | 2 | 2 (100.0%) | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 0 | 1 (100.0%) | 0 |

VASCULAR DISORDERS

| | | | | | | | | | |
|---|---|---|---|---|---|---|---|---|---|
| ALL EVENTS | A) PLACEBO | 32 | 0 | Treatment | 0.015* | 0 | 0 | 0 | 0 |
| | B) MS 60 mg | 28 | 4 (14.3%) | A-B | 0.026* | 4 | 4 (100.0%) | 0 | 0 |
| | C) NTX 0.01 mg | 30 | 1 (3.3%) | A-F | 0.004** | 1 | 1 (100.0%) | 0 | 0 |
| | D) MS 60 mg/NTX 0.001 mg | 18 | 0 | C-F | 0.025* | 0 | 0 | 0 | 0 |
| | E) MS 60 mg/NTX 0.01 mg | 28 | 3 (10.7%) | D-F | 0.028* | 3 | 1 (33.3%) | 2 (66.7%) | 0 |
| | F) MS 60 mg/NTX 0.1 mg | 26 | 6 (23.1%) | | | 7 | 3 (42.9%) | 4 (57.1%) | 0 |
| FLUSHING | A) PLACEBO | 32 | 0 | Treatment | 0.438 | 0 | 0 | 0 | 0 |
| | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 1 (3.6%) | | | 1 | 1 (100.0%) | 0 | 0 |
| | F) MS 60 mg/NTX 0.1 mg | 26 | 0 | | | 0 | 0 | 0 | 0 |
| HOT FLUSHES | A) PLACEBO | 32 | 0 | Treatment | 0.384 | 0 | 0 | 0 | 0 |
| NOS | B) MS 60 mg | 28 | 0 | | | 0 | 0 | 0 | 0 |
| | C) NTX 0.01 mg | 30 | 0 | | | 0 | 0 | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 0 | 1 (100.0%) | 0 |
| HYPERTENSION | A) PLACEBO | 32 | 0 | Treatment | 0.721 | 0 | 0 | 0 | 0 |
| NOS | B) MS 60 mg | 28 | 1 (3.6%) | | | 1 | 1 (100.0%) | 0 | 0 |
| | C) NTX 0.01 mg | 30 | 1 (3.3%) | | | 1 | 1 (100.0%) | 0 | 0 |
| | D) MS 60 mg/NTX 0.001 mg | 18 | 0 | | | 0 | 0 | 0 | 0 |
| | E) MS 60 mg/NTX 0.01 mg | 28 | 0 | | | 0 | 0 | 0 | 0 |
| | F) MS 60 mg/NTX 0.1 mg | 26 | 1 (3.8%) | | | 1 | 1 (100.0%) | 0 | 0 |
| VASODILATA- | A) PLACEBO | 32 | 0 | Treatment | 0.015* | 0 | 0 | 0 | 0 |
| TION | B) MS 60 mg | 28 | 3 (10.7%) | A-F | 0.009** | 3 | 3 (100.0%) | 0 | 0 |
| | C) NTX 0.01 mg | 30 | 0 | C-F | 0.011* | 0 | 0 | 0 | 0 |
| | D) MS 60 mg/NTX 0.001 mg | 18 | 0 | D-F | 0.048* | 0 | 0 | 0 | 0 |
| | E) MS 60 mg/NTX 0.01 mg | 28 | 2 (7.1%) | | | 2 | 0 | 2 (100.0%) | 0 |
| | F) MS 60 mg/NTX 0.1 mg | 26 | 5 (19.2%) | | | 5 | 2 (40.0%) | 3 (60.0%) | 0 |

[1] P-VALUES ARE FROM CHISQ TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
*, **, ***P-VALUE <=0.05, <=0.01, or <=0.001 RESPECTIVELY.

TABLE 52B
Selected Adverse Events
Intent-To-Treat Population, Female Patients

| BODY | | | | | | | | | |
|---|---|---|---|---|---|---|---|---|---|
| SYSTEM | | TOTAL | NO. OF | | | NO. | | | |
| ADVERSE | | NO. OF | SUBJECTS | | P-VALUE | OF | SEVERITY [2] | | |
| EVENTS | TREATMENT | SUBJECTS | W/EVENT | SOURCE | [1] | EVENTS | Mild | Moderate | Severe |
| DIZZINESS | A) PLACEBO | 32 | 1 (3.1%) | Treatment | <0.001*** | 1 | 0 | 1 (100.0%) | 0 |
| (EXC VERTIGO) | B) MS 60 mg | 28 | 16 (57.1%) | A-B | <0.001*** | 18 | 3 (16.7%) | 12 (66.7%) | 3 (16.7%) |
| | C) NTX 0.01 mg | 30 | 2 (6.7%) | A-D | <0.001*** | 2 | 2 (100.0%) | 0 | 0 |
| | D) MS 60 mg/ | 18 | 9 (50.0%) | A-E | <0.001*** | 9 | 3 (33.3%) | 6 (66.7%) | 0 |
| | NTX 0.001 mg | | | | | | | | |
| | E) MS 60 mg/ | 28 | 12 (42.9%) | A-F | 0.001** | 14 | 5 (35.7%) | 8 (57.1%) | 1 (7.1%) |
| | NTX 0.01 mg | | | | | | | | |
| | F) MS 60 mg/ | 26 | 9 (34.6%) | B-C | <0.001*** | 10 | 3 (30.0%) | 5 (50.0%) | 2 (20.0%) |
| | NTX 0.1 mg | | | | | | | | |
| | | | | C-D | <0.001*** | | | | |
| | | | | C-E | 0.001** | | | | |
| | | | | C-F | 0.008** | | | | |
| NAUSEA | A) PLACEBO | 32 | 5 (15.6%) | Treatment | <0.001*** | 6 | 2 (33.3%) | 1 (16.7%) | 3 (50.0%) |
| | B) MS 60 mg | 28 | 17 (60.7%) | A-B | <0.001*** | 21 | 5 (23.8%) | 12 (57.1%) | 4 (19.0%) |
| | C) NTX 0.01 mg | 30 | 9 (30.0%) | A-D | <0.001*** | 10 | 3 (30.0%) | 5 (50.0%) | 2 (20.0%) |
| | D) MS 60 mg/ | 18 | 16 (88.9%) | A-E | <0.001*** | 16 | 4 (25.0%) | 9 (56.3%) | 3 (18.8%) |
| | NTX 0.001 mg | | | | | | | | |
| | E) MS 60 mg/ | 28 | 21 (75.0%) | A-F | <0.001*** | 25 | 7 (28.0%) | 10 (40.0%) | 8 (32.0%) |
| | NTX 0.01 mg | | | | | | | | |
| | F) MS 60 mg/ | 26 | 16 (61.5%) | B-C | 0.018* | 18 | 1 (5.6%) | 15 (83.3%) | 2 (11.1%) |
| | NTX 0.1 mg | | | | | | | | |
| | | | | B-D | 0.038* | | | | |
| | | | | C-D | <0.001*** | | | | |
| | | | | C-E | <0.001*** | | | | |
| | | | | C-F | 0.017* | | | | |
| | | | | D-F | 0.045* | | | | |
| SOMNOLENCE | A) PLACEBO | 32 | 0 | Treatment | <0.001*** | 0 | 0 | 0 | 0 |
| | B) MS 60 mg | 28 | 8 (28.6%) | A-B | 0.001** | 9 | 1 (11.1%) | 6 (66.7%) | 2 (22.2%) |
| | C) NTX 0.01 mg | 30 | 0 | A-E | 0.012* | 0 | 0 | 0 | 0 |
| | D) MS 60 mg/ | 18 | 2 (11.1%) | A-F | <0.001*** | 2 | 0 | 2 (100.0%) | 0 |
| | NTX 0.001 mg | | | | | | | | |
| | E) MS 60 mg/ | 28 | 5 (17.9%) | B-C | 0.001** | 5 | 3 (60.0%) | 2 (40.0%) | 0 |
| | NTX 0.01 mg | | | | | | | | |
| | F) MS 60 mg/ | 26 | 8 (30.8%) | C-E | 0.015* | 8 | 5 (62.5%) | 2 (25.0%) | 1 (12.5%) |
| | NTX 0.1 mg | | | | | | | | |
| | | | | C-F | 0.001** | | | | |
| VOMITING | A) PLACEBO | 32 | 3 (9.4%) | Treatment | <0.001*** | 3 | 1 (33.3%) | 0 | 2 (66.7%) |
| NOS | B) MS 60 mg | 28 | 16 (57.1%) | A-B | <0.001*** | 17 | 1 (5.9%) | 5 (29.4%) | 11 (64.7%) |
| | C) NTX 0.01 mg | 30 | 7 (23.3%) | A-D | <0.001*** | 7 | 1 (14.3%) | 1 (14.3%) | 5 (71.4%) |
| | D) MS 60 mg/ | 18 | 15 (83.3%) | A-E | <0.001*** | 16 | 1 (6.3%) | 3 (18.8%) | 12 (75.0%) |
| | NTX 0.001 mg | | | | | | | | |
| | E) MS 60 mg/ | 28 | 17 (60.7%) | A-F | <0.001*** | 18 | 3 (16.7%) | 3 (16.7%) | 12 (66.7%) |
| | NTX 0.01 mg | | | | | | | | |
| | F) MS 60 mg/ | 26 | 16 (61.5%) | B-C | 0.008** | 21 | 2 (9.5%) | 5 (23.8%) | 14 (66.7%) |
| | NTX 0.1 mg | | | | | | | | |
| | | | | C-D | <0.001*** | | | | |
| | | | | C-E | 0.003** | | | | |
| | | | | C-F | 0.003** | | | | |

[1] P-VALUES ARE FROM CHISQ TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
NOTE:
ADVERSE EVENTS RELATED TO STUDY DRUG: RELATIONSHIP TO STUDY DRUG = ‘SUSPECT’ OR ‘PROBABLE’.
*, **, ***P-VALUE <=0.05, <=0.01, or <=0.001 RESPECTIVELY.

TABLE 52C
Adverse Events By Body System And
Intent-To-Treat Population, Male Patients

| BODY SYSTEM | | | | | | | | | |
|---|---|---|---|---|---|---|---|---|---|
| ADVERSE | | TOTAL | | | | No. | | | |
| EVENTS | | NO. OF | NO. OF | | | of | | | |
| (COSTART | | PA- | PATIENTS | | P-Value | E- | SEVERITY [2] | | |
| ENGLISH) | TREATMENT | TIENTS | W/EVENT | SOURCE | [1] | vents | Mild | Moderate | Severe |

TOTAL NUMBER OF EVENTS
ADVERSE EVENTS
(ALL BODY SYSTEMS)

| | | | | | | | | | |
|---|---|---|---|---|---|---|---|---|---|
| All EVENTS | A) PLACEBO | 19 | 13 (68.4%) | Treatment | <0.001*** | 26 | 10 (38.5%) | 12 (46.2%) | 4 (15.4%) |
| | B) MS 60 mg | 25 | 20 (80.0% | A-C | 0.026* | 59 | 30 (50.8%) | 22 (37.3%) | 7 (11.9%) |
| | C) NTX 0.01 mg | 21 | 7 (33.3%) | B-C | 0.001** | 13 | 5 (38.5%) | 6 (46.2%) | 2 (15.4%) |
| | D) MS 60 mg/NTX 0.001 mg | 32 | 28 (87.5%) | C-D | <0.001*** | 75 | 32 (42.7%) | 29 (38.7%) | 14 (18.7%) |
| | E) MS 60 mg/NTX 0.001 mg | 23 | 20 (87.0%) | C-E | <0.001*** | 58 | 20 (34.5%) | 20 (34.5%) | 18 (31.0%) |
| | F) MS 60 mg/NTX 0.1 mg | 22 | 20 (90.9%) | C-F | <0.001*** | 57 | 21 (36.8%) | 21 (36.8%) | 15 (26.3%) |

CARDIAC DISORDERS

| | | | | | | | | | |
|---|---|---|---|---|---|---|---|---|---|
| ALL EVENTS | A) PLACEBO | 19 | 1 (5.3%) | Treatment | 0.590 | 1 | 1 (100.0%) | 0 | 0 |
| | B) MS 60 mg | 25 | 1 (4.0%) | | | 1 | 1 (100.0%) | 0 | 0 |
| | C) NTX 0.01 mg | 21 | 1 (4.8%) | | | 1 | 1 (100.0%) | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| BRADYCARDIA | A) PLACEBO | 19 | 1 (5.3%) | Treatment | 0.258 | 1 | 1 (100.0%) | 0 | 0 |
| NOS | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| TACHYCARDIA | A) PLACEBO | 19 | 0 | Treatment | 0.509 | 0 | 0 | 0 | 0 |
| NOS | B) MS 60 mg | 25 | 1 (4.0%) | | | 1 | 1 (100.0%) | 0 | 0 |
| | C) NTX 0.01 mg | 21 | 1 (4.8%) | | | 1 | 1 (100.0%) | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

EAR AND LABYRINTH DISORDERS

| | | | | | | | | | |
|---|---|---|---|---|---|---|---|---|---|
| ALL EVENTS | A) PLACEBO | 19 | 1 (5.3%) | Treatment | 0.685 | 1 | 0 | 1 (100.0%) | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |
| EARACHE | A) PLACEBO | 19 | 1 (5.3%) | Treatment | 0.685 | 1 | 0 | 1 (100.0%) | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |

EYE DISORDERS

| | | | | | | | | | |
|---|---|---|---|---|---|---|---|---|---|
| ALL EVENTS | A) PLACEBO | 19 | 1 (5.3%) | Treatment | 0.555 | 1 | 0 | 1 (100.0%) | 0 |
| | B) MS 60 mg | 25 | 4 (16.0%) | | | 4 | 4 (100.0%) | 0 | 0 |
| | C) NTX 0.01 mg | 21 | 1 (4.8%) | | | 1 | 0 | 1 (100.0%) | 0 |
| | D) MS 60 mg/NTX 0.001 mg | 32 | 5 (15.6%) | | | 5 | 4 (80.0%) | 0 | 1 (20.0%) |
| | E) MS 60 mg/NTX 0.01 mg | 23 | 3 (13.0%) | | | 3 | 2 (66.7%) | 0 | 1 (33.3%) |
| | F) MS 60 mg/NTX 0.1 mg | 22 | 1 (4.5%) | | | 1 | 1 (100.0%) | 0 | 0 |
| CONJUNCTIVI- | A) PLACEBO | 19 | 0 | Treatment | 0.511 | 0 | 0 | 0 | 0 |
| TIS NEC | B) MS 60 mg | 25 | 3 (12.0%) | | | 3 | 3 (100.0%) | 0 | 0 |
| | C) NTX 0.01 mg | 21 | 1 (4.8%) | | | 1 | 0 | 1 (100.0%) | 0 |
| | D) MS 60 mg/NTX 0.001 mg | 32 | 4 (12.5%) | | | 4 | 4 (100.0%) | 0 | 0 |
| | E) MS 60 mg/NTX 0.01 mg | 23 | 3 (13.0%) | | | 3 | 2 (66.7%) | 0 | 1 (33.3%) |
| | F) MS 60 mg/NTX 0.1 mg | 22 | 1 (4.5%) | | | 1 | 1 (100.0%) | 0 | 0 |
| PHOTOPHOBIA | A) PLACEBO | 19 | 1 (5.3%) | Treatment | 0.258 | 1 | 0 | 1 (100.0%) | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| TIRED EYES | A) PLACEBO | 19 | 0 | Treatment | 0.629 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 0 | 0 | 1 (100.0%) |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| VISION | A) PLACEBO | 19 | 0 | Treatment | 0.451 | 0 | 0 | 0 | 0 |
| BLURRED | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

GASTROINTESTINAL DISORDERS

| | | | | | | | | | |
|---|---|---|---|---|---|---|---|---|---|
| ALL EVENTS | A) PLACEBO | 19 | 3 (15.8%) | Treatment | <0.001*** | 5 | 1 (20.0%) | 1 (20.0%) | 3 (60.0%) |
| | B) MS 60 mg | 25 | 11 (44.0%) | A-B | 0.046* | 21 | 11 (52.4%) | 6 (28.6%) | 4 (19.0%) |
| | C) NTX 0.01 mg | 21 | 0 | A-D | 0.004** | 0 | 0 | 0 | 0 |
| | D) MS 60 mg/NTX 0.001 mg | 32 | 18 (56.3%) | A-F | 0.004** | 31 | 9 29.0%) | 13 (41.9%) | 9 (29.0%) |
| | E) MS 60 mg/NTX 0.01 mg | 23 | 10 (43.5%) | B-C | <0.001*** | 18 | 3 (16.7%) | 5 (27.8%) | 10 (55.6%) |
| | F) MS 60 mg/NTX 0.1 mg | 22 | 13 (59.1%) | C-D | <0.001*** | 23 | 7 (30.4%) | 6 (26.1%) | 10 (43.5%) |
| | | | | C-E | <0.001*** | | | | |
| | | | | C-F | <0.001*** | | | | |
| ABDOMINAL | A) PLACEBO | 19 | 1 (5.3%) | Treatment | 0.441 | 1 | 0 | 0 | 1 (100.0%) |
| PAIN NOS | B) MS 60 mg | 25 | 2 (8.0%) | | | 2 | 1 (50.0%) | 1 (50.0%) | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| ABDOMINAL | A) PLACEBO | 19 | 0 | Treatment | 0.358 | 0 | 0 | 0 | 0 |
| PAIN UPPER | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 0 | 1 (100.0%) | 0 |
| DYSPHAGIA | A) PLACEBO | 19 | 1 (5.3%) | Treatment | 0.547 | 1 | 0 | 0 | 1 (100.0%) |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 0 | 0 | 1 (100.0%) |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| HICCUPS | A) PLACEBO | 19 | 0 | Treatment | 0.390 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |
| NAUSEA | A) PLACEBO | 19 | 2 (10.5%) | Treatment | 0.001** | 2 | 1 (50.0%) | 1 (50.0%) | 0 |
| | B) MS 60 mg | 25 | 10 (40.0%) | A-B | 0.029* | 10 | 7 (70.0%) | 3 (30.0%) | 0 |
| | C) NTX 0.01 mg | 21 | 0 | A-D | 0.013* | 0 | 0 | 0 | 0 |
| | D) MS 60 mg/NTX 0.001 mg | 32 | 14 (43.8%) | A-F | 0.014* | 15 | 5 (33.3%) | 7 (46.7%) | 3 (20.0%) |
| | E) MS 60 mg/NTX 0.01 mg | 23 | 6 (26.1%) | B-C | 0.001** | 6 | 2 (33.3%) | 2 (33.3%) | 2 (33.3%) |
| | F) MS 60 mg/NTX 0.1 mg | 22 | 10 (45.5%) | C-D | <0.001*** | 10 | 6 (60.0%) | 4 40.0%) | 0 |
| | | | | C-E | 0.011* | | | | |
| | | | | C-F | <0.001*** | | | | |
| SORE THROAT | A) PLACEBO | 19 | 0 | Treatment | 0.629 | 0 | 0 | 0 | 0 |
| NOS | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| VOMITING NOS | A) PLACEBO | 19 | 1 (5.3%) | Treatment | <0.001*** | 1 | 0 | 0 | 1 (100.0%) |
| | B) MS 60 mg | 25 | 9 (36.0%) | A-B | 0.015* | 9 | 3 (33.3%) | 2 (22.2%) | 4 (44.4%) |
| | C) NTX 0.01 mg | 21 | 0 | A-D | 0.010* | 0 | 0 | 0 | 0 |
| | D) MS 60 mg/NTX 0.001 mg | 32 | 12 (37.5%) | A-E | 0.020* | 13 | 2 (15.4%) | 6 (46.2%) | 5 (38.5%) |
| | E) MS 60 mg/NTX 0.01 mg | 23 | 8 (34.8%) | A-F | 0.001** | 11 | 1 (9.1%) | 2 (18.2%) | 8 (72.7%) |
| | F) MS 60 mg/NTX 0.1 mg | 22 | 11 (50.0%) | B-C | 0.002** | 12 | 1 (8.3%) | 1 (8.3%) | 10 (83.3%) |
| | | | | C-D | 0.001** | | | | |
| | | | | C-E | 0.002** | | | | |
| | | | | C-F | <0.001*** | | | | |

GENERAL DISORDERS AND ADMINISTRATION SITE CONDITIONS

| | | | | | | | | | |
|---|---|---|---|---|---|---|---|---|---|
| ALL EVENTS | A) PLACEBO | 19 | 3 (15.8%) | Treatment | 0.280 | 3 | 1 (33.3%) | 2 (66.7%) | 0 |
| | B) MS 60 mg | 25 | 5 (20.0%) | A-E | 0.047* | 5 | 2 (40.0%) | 2 (40.0%) | 1 (20.0%) |
| | C) NTX 0.01 mg | 21 | 1 (4.8%) | B-E | 0.023* | 2 | 0 | 1 (50.0%) | 1 (50.0%) |
| | D) MS 60 mg/NTX 0.001 mg | 32 | 4 (12.5%) | | | 4 | 3 (75.0%) | 1 (25.0%) | 0 |
| | E) MS 60 mg/NTX 0.01 mg | 23 | 0 | | | 0 | 0 | 0 | 0 |
| | F) MS 60 mg/NTX 0.1 mg | 22 | 3 (13.6%) | | | 3 | 2 (66.6%) | 1 (33.3%) | 0 |
| ASTHENIA | A) PLACEBO | 19 | 0 | Treatment | 0.013* | 0 | 0 | 0 | 0 |
| | B) MS 60 mg | 25 | 3 (12.0%) | B-D | 0.044* | 3 | 1 (33.3%) | 2 (66.7%) | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| FEELING | A) PLACEBO | 19 | 0 | Treatment | 0.451 | 0 | 0 | 0 | 0 |
| ABNORMAL | B) MS 60 mg | 25 | 1 (4.0%) | | | 1 | 0 | 0 | 1 (100.0%) |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| FEELING HOT | A) PLACEBO | 19 | 0 | Treatment | 0.600 | 0 | 0 | 0 | 0 |
| | B) MS 60 mg | 25 | 0 | | | 0 | 0 | 0 | 0 |
| | C) NTX 0.01 mg | 21 | 1 (4.8%) | | | 1 | 0 | 0 | 1 (100.0%) |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| PAIN NOS | A) PLACEBO | 19 | 0 | Treatment | 0.624 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 0 | 1 (100.0%) | 0 |
| PYREXIA | A) PLACEBO | 19 | 1 (5.3%) | Treatment | 0.839 | 1 | 1 (100.0%) | 0 | 0 |
| | B) MS 60 mg | 25 | 1 (4.0) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 1 (100.0%) | 0 | 0 |
| RIGORS | A) PLACEBO | 19 | 2 (10.5%) | Treatment | 0.264 | 2 | 0 | 2 (100.0%) | 0 |
| | B) MS 60 mg | 25 | 0 | | | 0 | 0 | 0 | 0 |
| | C) NTX 0.01 mg | 21 | 1 (4.58%) | | | 1 | 0 | 1 (100.0%) | 0 |
| | D) MS 60 mg/NTX 0.001 mg | 32 | 1 (3.1%) | | | 1 | 0 | 1 (100.0%) | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| WEAKNESS | A) PLACEBO | 19 | 0 | Treatment | 0.358 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 1 (100.0%) | 0 | 0 |

INFECTIONS AND INFESTATIONS

| | | | | | | | | | |
|---|---|---|---|---|---|---|---|---|---|
| ALL EVENTS | A) PLACEBO | 19 | 4 (21.1%) | Treatment | 0.654 | 6 | 4 (66.7%) | 1 (16.7%) | 1 (16.7%) |
| | B) MS 60 mg | 25 | 2 (8.0%) | | | 2 | 0 | 0 | 2 (100.0%) |
| | C) NTX 0.01 mg | 21 | 2 (9.5%) | | | 2 | 0 | 2 (100.0%) | 0 |
| | D) MS 60 mg/NTX 0.001 mg | 32 | 2 (6.3%) | | | 2 | 0 | 0 | 2 (100.0%) |
| | E) MS 60 mg/NTX 0.01 mg | 23 | 2 (8.7%) | | | 3 | 0 | 0 | 3 (100.0%) |
| | F) MS 60 mg/NTX 0.1 mg | 22 | 2 (9.1%) | | | 2 | 0 | 1 (50.0%) | 1 (50.0%) |
| CELLULITIS | A) PLACEBO | 19 | 0 | Treatment | 0.358 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 0 | 0 | 1 (100.0%) |
| DRY SOCKET | A) PLACEBO | 19 | 1 (5.3%) | Treatment | 0.848 | 1 | 0 | 1 (100.0%) | 0 |
| NOS | B) MS 60 mg | 25 | 1 (4.0%) | | | 1 | 0 | 0 | 1 (100.0%) |
| | C) NTX 0.01 mg | 21 | 1 (4.8%) | | | 1 | 0 | 1 (100.0%) | 0 |
| | D) MS 60 mg/NTX 0.001 mg | 32 | 2 (6.3%) | | | 2 | 0 | 0 | 2 (100.0%) |
| | E) MS 60 mg/NTX 0.01 mg | 23 | 2 (8.7%) | | | 2 | 0 | 0 | 2 (100.0%) |
| | F) MS 60 mg/NTX 0.1 mg | 22 | 0 | | | 0 | 0 | 0 | 0 |
| NASO- | A) PLACEBO | 19 | 0 | Treatment | 0.451 | 0 | 0 | 0 | 0 |
| PHARYNGITIS | B) MS 60 mg | 25 | 1 (4.0%) | | | 1 | 0 | 0 | 1 (100.0%) |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| ORAL | A) PLACEBO | 19 | 0 | Treatment | 0.390 | 0 | 0 | 0 | 0 |
| INFECTION | B) MS 60 mg | 25 | 0 | | | 0 | 0 | 0 | 0 |
| NEC | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 0 | 1 (100.0%) |
| | F) MS 60 mg/NTX 0.1 mg | 22 | 0 | | | 0 | 0 | 0 | 0 |
| PHARYNGITIS | A) PLACEBO | 19 | 2 (10.5%) | Treatment | 0.093 | 4 | 3 (75.0%) | 0 | 1 (25.0%) |
| NOS | B) MS 60 mg | 25 | 0 | | | 0 | 0 | 0 | 0 |
| | C) NTX 0.01 mg | 21 | 1 (4.8%) | | | 1 | 0 | 1 (100.0%) | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| TOOTH | A) PLACEBO | 19 | 0 | Treatment | 0.358 | 0 | 0 | 0 | 0 |
| INFECTION | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 0 | 1 (100.0%) | 0 |
| UPPER | A) PLACEBO | 19 | 1 (5.3%) | Treatment | 0.258 | 1 | 1 (100.0%) | 0 | 0 |
| RESPIRATORY | B) MS 60 mg | 25 | 0 | | | 0 | 0 | 0 | 0 |
| TRACT | C) NTX 0.01 mg | 21 | 0 | | | 0 | 0 | 0 | 0 |
| INFECTION | D) MS 60 mg/NTX 0.001 mg | 32 | 0 | | | 0 | 0 | 0 | 0 |
| NOS | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

INJURY AND
POISONING

| | | | | | | | | | |
|---|---|---|---|---|---|---|---|---|---|
| ALL EVENTS | A) PLACEBO | 19 | 1 (5.3%) | Treatment | 0.258 | 1 | 0 | 1 (100.0%) | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| HYPOTHERMIA | A) PLACEBO | 19 | 1 (5.3%) | Treatment | 0.258 | 1 | 0 | 1 (100.0%) | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

INVESTIGATIONS

| | | | | | | | | | |
|---|---|---|---|---|---|---|---|---|---|
| ALL EVENTS | A) PLACEBO | 19 | 0 | Treatment | 0.390 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |
| HAEMATURIA | A) PLACEBO | 19 | 0 | Treatment | 0.390 | 0 | 0 | 0 | 0 |
| PRESENT | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |

MUSCULOSKELETAL, CONNECTIVE TISSUE AND BONE DISORDERS

| | | | | | | | | | |
|---|---|---|---|---|---|---|---|---|---|
| ALL EVENTS | A) PLACEBO | 19 | 0 | Treatment | 0.090 | 0 | 0 | 0 | 0 |
| | B) MS 60 mg | 25 | 2 (8.0%) | | | 2 | 0 | 2 (100.0%) | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| NECK | A) PLACEBO | 19 | 0 | Treatment | 0.451 | 0 | 0 | 0 | 0 |
| STIFFNESS | B) MS 60 mg | 25 | 1 (4.0%) | | | 1 | 0 | 1 (100.0%) | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| SENSATION OF | A) PLACEBO | 19 | 0 | Treatment | 0.451 | 0 | 0 | 0 | 0 |
| HEAVINESS | B) MS 60 mg | 25 | 1 (4.0) | | | 1 | 0 | 1 (100.0%) | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

NERVOUS SYSTEM
DISORDERS

| | | | | | | | | | |
|---|---|---|---|---|---|---|---|---|---|
| ALL EVENTS | A) PLACEBO | 19 | 6 (31.6%) | Treatment | 0.005** | 6 | 3 (50.0%) | 3 (50.0%) | 0 |
| | B) MS 60 mg | 25 | 13 (52.0%) | A-D | 0.032* | 15 | 5 (33.3%) | 10 (66.7%) | 0 |
| | C) NTX 0.01 mg | 21 | 4 (19.0%) | A-F | 0.019* | 4 | 2 (50.0%) | 1 (25.0%)) | 1 (25.0%) |
| | D) MS 60 mg/NTX 0.001 mg | 32 | 20 (62.5%) | B-C | 0.021* | 26 | 12 (46.2%) | 12 (46.2%) | 2 (7.7%) |
| | E) MS 60 mg/NTX 0.01 mg | 23 | 14 (60.9%) | C-D | 0.001** | 21 | 11 (52.4%) | 7 (33.3%) | 3 (14.3%) |
| | F) MS 60 mg/NTX 0.1 mg | 22 | 15 (68.2%) | C-E | 0.004** | 21 | 9 (42.9%) | 10 (47.6%) | 2 (9.5%) |
| | | | | C-F | 0.001** | | | | |
| DIZZINESS | A) PLACEBO | 19 | 1 (5.3%) | Treatment | 0.008** | 1 | 0 | 1 (100.0%) | 0 |
| (EXC VERTIGO) | B) MS 60 mg | 25 | 3 (12.0%) | A-D | 0.046* | 3 | 1 (33.3%) | 2 (66.7%) | 0 |
| | C) NTX 0.01 mg | 21 | 0 | A-E | 0.020* | 0 | 0 | 0 | 0 |
| | D) MS 60 mg/NTX 0.001 mg | 32 | 9 (28.1%) | A-F | 0.032* | 10 | 4 (40.0%) | 5 (50.0%) | 1 (10.0%) |
| | E) MS 60 mg/NTX 0.01 mg | 23 | 8 (34.8%) | C-D | 0.007** | 9 | 5 (55.6%) | 4 (44.4%) | 0 |
| | F) MS 60 mg/NTX 0.1 mg | 22 | 7 (31.8%) | C-E | 0.002** | 9 | 4 (44.4%) | 4 (44.4%) | 1 (11.1%) |
| | | | | C-F | 0.004** | | | | |
| HEADACHE | A) PLACEBO | 19 | 3 (15.8%) | Treatment | 0.444 | 3 | 2 (66.7%) | 1 (33.3%) | 0 |
| NOS | B) MS 60 mg | 25 | 6 (24.0%) | | | 7 | 2 (28.6%) | 5 (71.4%) | 0 |
| | C) NTX 0.01 mg | 21 | 3 (14.3%) | | | 3 | 1 (33.3%) | 1 (33.3%) | 1 (33.3%) |
| | D) MS 60 mg/NTX 0.001 mg | 32 | 6 (18.8%) | | | 7 | 1 (14.3%) | 5 (71.4%) | 1 (14.3%) |
| | E) MS 60 mg/NTX 0.01 mg | 23 | 2 (8.7%) | | | 2 | 1 (50.0%) | 0 | 1 (50.0%) |
| | F) MS 60 mg/NTX 0.1 mg | 22 | 7 (31.8%) | | | 7 | 4 (57.1%) | 3 (42.9%) | 0 |
| HYPERTONIA | A) PLACEBO | 19 | 0 | Treatment | 0.390 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |
| HYPO- | A) PLACEBO | 19 | 0 | Treatment | 0.390 | 0 | 0 | 0 | 0 |
| AESTHESIA | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |
| MIGRAINE NOS | A) PLACEBO | 19 | 0 | Treatment | 0.390 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 0 | 1 (100.0%) |
| | F) MS 60 mg/NTX 0.1 mg | 22 | 0 | | | 0 | 0 | 0 | 0 |
| MUSCLE | A) PLACEBO | 19 | 0 | Treatment | 0.451 | 0 | 0 | 0 | 0 |
| SPASTICITY | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| PARAESTHESIA | A) PLACEBO | 19 | 0 | Treatment | 0.629 | 0 | 0 | 0 | 0 |
| CIRCUMORAL | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| PARAESTHESIA | A) PLACEBO | 19 | 2 (10.5%) | Treatment | 0.510 | 2 | 1 (50.0%) | 1 (50.0%) | 0 |
| NEC | B) MS 60 mg | 25 | 0 | | | 0 | 0 | 0 | 0 |
| | C) NTX 0.01 mg | 21 | 1 (4.8%) | | | 1 | 1 (100.0%) | 0 | 0 |
| | D) MS 60 mg/NTX 0.001 mg | 32 | 2 (6.3%) | | | 2 | 2 (100.0%) | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |
| SOMMOLENCE | A) PLACEBO | 19 | 0 | Treatment | 0.209 | 0 | 0 | 0 | 0 |
| | B) MS 60 mg | 25 | 3 (12.0%) | C-F | 0.040* | 4 | 1 (25.0%) | 3 (75.0%) | 0 |
| | C) NTX 0.01 mg | 21 | 0 | | | 0 | 0 | 0 | 0 |
| | D) MS 60 mg/NTX 0.001 mg | 32 | 5 (15.6%) | | | 6 | 4 (66.7%) | 2 (33.3%) | 0 |
| | E) MS 60 mg/NTX 0.01 mg | 23 | 3 (13.0%) | | | 3 | 1 (33.3%) | 2 (66.7%) | 0 |
| | F) MS 60 mg/NTX 0.1 mg | 22 | 4 (18.2%) | | | 4 | 1 (25.0%) | 3 (75.0%) | 0 |
| SYNCOPE | A) PLACEBO | 19 | 0 | Treatment | 0.390 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 0 | 1 (100.0%) |
| | F) MS 60 mg/NTX 0.1 mg | 22 | 0 | | | 0 | 0 | 0 | 0 |
| TENSION | A) PLACEBO | 19 | 0 | Treatment | 0.358 | 0 | 0 | 0 | 0 |
| HEADACHES | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 0 | 0 | 1 (100.0%) |
| TREMOR NEC | A) PLACEBO | 19 | 0 | Treatment | 0.062 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 2 (8.7%) | | | 2 | 1 (50.0%) | 1 (50.0%) | 0 |
| | F) MS 60 mg/NTX 0.1 mg | 22 | 0 | | | 0 | 0 | 0 | 0 |

PSYCHIATRIC
DISORDERS

| | | | | | | | | | |
|---|---|---|---|---|---|---|---|---|---|
| ALL EVENTS | A) PLACEBO | 19 | 1 (5.3%) | Treatment | 0.593 | 1 | 0 | 1 (100.0%) | 0 |
| | B) MS 60 mg | 25 | 2 (8.0%) | | | 2 | 1 (50.0%) | 1 (50.0%) | 0 |
| | C) NTX 0.01 mg | 21 | 0 | | | 0 | 0 | 0 | 0 |
| | D) MS 60 mg/NTX 0.001 mg | 32 | 2 (6.3%) | | | 2 | 0 | 2 (100.0%) | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 3 (13.6%) | | | 3 | 1 (33.3%) | 1 (33.3%) | 1 (33.3%) |
| DISORIENTA- | A) PLACEBO | 19 | 0 | Treatment | 0.390 | 0 | 0 | 0 | 0 |
| TION | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |
| DISSOCIATION | A) PLACEBO | 19 | 0 | Treatment | 0.358 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 0 | 0 | 1 (100.0%) |
| EUPHORIC | A) PLACEBO | 19 | 0 | Treatment | 0.400 | 0 | 0 | 0 | 0 |
| MOOD | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 0 | 1 (100.0%) | 0 |
| | E) MS 60 mg/NTX 0.01 mg | 23 | 0 | | | 0 | 0 | 0 | 0 |
| | F) MS 60 mg/NTX 0.1 mg | 22 | 2 (9.1%) | | | 2 | 1 (50.0%) | 1 (50.0%) | 0 |
| NERVOUSNESS | A) PLACEBO | 19 | 1 (5.3%) | Treatment | 0.711 | 1 | 0 | 1 (100.0%) | 0 |
| | B) MS 60 mg | 25 | 1 (4.0%) | | | 1 | 0 | 1 (100.0%) | 0 |
| | C) NTX 0.01 mg | 21 | 0 | | | 0 | 0 | 0 | 0 |
| | D) MS 60 mg/NTX 0.001 mg | 32 | 1 (3.1%) | | | 1 | 0 | 1 (100.0%) | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

RENAL AND URINARY DISORDERS

| | | | | | | | | | |
|---|---|---|---|---|---|---|---|---|---|
| ALL EVENTS | A) PLACEBO | 19 | 0 | Treatment | 0.551 | 0 | 0 | 0 | 0 |
| | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |
| URINARY | A) PLACEBO | 19 | 0 | Treatment | 0.551 | 0 | 0 | 0 | 0 |
| RETENTION | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |

REPRODUCTIVE SYSTEM AND BREAST DISORDERS

| | | | | | | | | | |
|---|---|---|---|---|---|---|---|---|---|
| ALL EVENTS | A) PLACEBO | 19 | 0 | Treatment | 0.390 | 0 | 0 | 0 | 0 |
| | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 2 | 0 | 1 (50.0%) | 1 (50.0%) |
| | F) MS 60 mg/NTX 0.1 mg | 22 | 0 | | | 0 | 0 | 0 | 0 |
| PROSTATIC | A) PLACEBO | 19 | 0 | Treatment | 0.390 | 0 | 0 | 0 | 0 |
| DISORDER NOS | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |
| TESTICULAR | A) PLACEBO | 19 | 0 | Treatment | 0.390 | 0 | 0 | 0 | 0 |
| DISORDER NOS | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 0 | 0 | 1 (100.0%) |
| | F) MS 60 mg/NTX 0.1 mg | 22 | 0 | | | 0 | 0 | 0 | 0 |

RESPIRATORY, THORACIC AND MEDIASTINAL DISORDERS

| | | | | | | | | | |
|---|---|---|---|---|---|---|---|---|---|
| ALL EVENTS | A) PLACEBO | 19 | 0 | Treatment | 0.643 | 0 | 0 | 0 | 0 |
| | B) MS 60 mg | 25 | 1 (4.0%) | | | 1 | 1 (100.0%) | 0 | 0 |
| | C) NTX 0.01 mg | 21 | 1 (4.8%) | | | 1 | 1 (100.0%) | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 1 (100.0%) | 0 | 0 |
| EPISTAXIS | A) PLACEBO | 19 | 0 | Treatment | 0.325 | 0 | 0 | 0 | 0 |
| | B) MS 60 mg | 25 | 0 | | | 0 | 0 | 0 | 0 |
| | C) NTX 0.01 mg | 21 | 1 (4.8%) | | | 1 | 1 (100.0%) | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| NECK | A) PLACEBO | 19 | 0 | Treatment | 0.358 | 0 | 0 | 0 | 0 |
| TIGHTNESS | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 1 (4.5%) | | | 1 | 1 (100.0%) | 0 | 0 |
| RHINITIS NOS | A) PLACEBO | 19 | 0 | Treatment | 0.451 | 0 | 0 | 0 | 0 |
| | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |

SKIN & SUBCUTANEOUS TISSUE DISORDERS

| | | | | | | | | | |
|---|---|---|---|---|---|---|---|---|---|
| ALL EVENTS | A) PLACEBO | 19 | 0 | Treatment | 0.122 | 0 | 0 | 0 | 0 |
| | B) MS 60 mg | 25 | 2 (8.0%) | D-E | 0.014* | 2 | 2 (100.0%) | 0 | 0 |
| | C) NTX 0.01 mg | 21 | 1 (4.8%) | | | 1 | 0 | 1 (100.0%) | 0 |
| | D) MS 60 mg/NTX 0.001 mg | 32 | 0 | | | 0 | 0 | 0 | 0 |
| | E) MS 60 mg/NTX 0.01 mg | 23 | 4 (17.4%) | | | 5 | 2 (40.0%) | 3 (60.0%) | 0 |
| | F) MS 60 mg/NTX 0.1 mg | 22 | 2 (9.1%) | | | 2 | 0 | 1 (50.0%) | 1 (50.0%) |
| ERYTHEMA | A) PLACEBO | 19 | 0 | Treatment | 0.451 | 0 | 0 | 0 | 0 |
| NEC | B) MS 60 mg | 25 | 1 (4.0%) | | | 1 | 1 (100.0%) | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| PHOTO- | A) PLACEBO | 19 | 0 | Treatment | 0.390 | 0 | 0 | 0 | 0 |
| SENSITIVITY | B) MS 60 mg | 25 | 0 | | | 0 | 0 | 0 | 0 |
| REACTION NOS | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 0 | | | 0 | 0 | 0 | 0 |
| PRURITUS NOS | A) PLACEBO | 19 | 0 | Treatment | 0.037* | 0 | 0 | 0 | 0 |
| | B) MS 60 mg | 25 | 0 | D-E | 0.035* | 0 | 0 | 0 | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 3 (13.0%) | | | 3 | 0 | 3 (100.0%) | 0 |
| | F) MS 60 mg/NTX 0.1 mg | 22 | 1 (4.5%) | | | 1 | 0 | 0 | 1 (100.0%) |
| SWEATING | A) PLACEBO | 19 | 0 | Treatment | 0.801 | 0 | 0 | 0 | 0 |
| INCREASED | B) MS 60 mg | 25 | 1 (4.0%) | | | 1 | 1 (100.0%) | 0 | 0 |
| | C) NTX 0.01 mg | 21 | 1 (4.8%) | | | 1 | 0 | 1 (100.0%) | 0 |
| | D) MS 60 mg/NTX 0.001 mg | 32 | 0 | | | 0 | 0 | 0 | 0 |
| | E) MS 60 mg/NTX 0.01 mg | 23 | 1 (4.3%) | | | 1 | 1 (100.0%) | 0 | 0 |
| | F) MS 60 mg/NTX 0.1 mg | 22 | 1 (4.5%) | | | 1 | 0 | 1 (100.0%) | 0 |

VASCULAR DISORDERS

| | | | | | | | | | |
|---|---|---|---|---|---|---|---|---|---|
| ALL EVENTS | A) PLACEBO | 19 | 1 (5.3%) | Treatment | 0.829 | 1 | 0 | 1 (100.0%) | 0 |
| | B) MS 60 mg | 25 | 3 (12.0%) | | | 3 | 2 (66.7%) | 1 (33.3%) | 0 |
| | C) NTX 0.01 mg | 21 | 1 (4.8%) | | | 1 | 1 (100.0%) | 0 | 0 |
| | D) MS 60 mg/NTX 0.001 mg | 32 | 4 (12.5%) | | | 4 | 3 (75.0%) | 1 (25.0%) | 0 |
| HOT FLUSHES | A) PLACEBO | 19 | 0 | Treatment | 0.451 | 0 | 0 | 0 | 0 |
| NOS | B) MS 60 mg | 25 | 1 (4.0%) | | | 1 | 0 | 1 (100.0%) | 0 |
| | C) NTX 0.01 mg | 21 | 0 | | | 0 | 0 | 0 | 0 |
| | D) MS 60 mg/NTX 0.001 mg | 32 | 0 | | | 0 | 0 | 0 | 0 |
| | E) MS 60 mg/NTX 0.01 mg | 23 | 0 | | | 0 | 0 | 0 | 0 |
| | F) MS 60 mg/NTX 0.1 mg | 22 | 0 | | | 0 | 0 | 0 | 0 |
| HYPER- | A) PLACEBO | 19 | 0 | Treatment | 0.170 | 0 | 0 | 0 | 0 |
| TENSION NOS | B) MS 60 mg | 25 | 0 | | | 0 | 0 | 0 | 0 |
| | C) NTX 0.01 mg | 21 | 0 | | | 0 | 0 | 0 | 0 |
| | D) MS 60 mg/NTX 0.001 mg | 32 | 3 (9.4%) | | | 3 | 2 (66.7%) | 1 (33.3%) | 0 |
| | E) MS 60 mg/NTX 0.01 mg | 23 | 1 (4.3%) | | | 1 | 0 | 1 (100.0%) | 0 |
| | F) MS 60 mg/NTX 0.1 mg | 22 | 0 | | | 0 | 0 | 0 | 0 |
| VASODILATA- | A) PLACEBO | 19 | 1 (5.3%) | Treatment | 0.979 | 1 | 0 | 1 (100.0%) | 0 |
| TION | B) MS 60 mg | 25 | 2 (8.0%) | | | 2 | 2 (100.0%) | 0 | 0 |
| | C) NTX 0.01 mg | 21 | 1 (4.8%) | | | 1 | 1 (100.0%) | 0 | 0 |
| | D) MS 60 mg/NTX 0.001 mg | 32 | 1 (3.1%) | | | 1 | 1 (100.0%) | 0 | 0 |
| | E) MS 60 mg/NTX 0.01 mg | 23 | 1 4.3%) | | | 1 | 0 | 1 (100.0%) | 0 |
| | F) MS 60 mg/NTX 0.1 mg | 22 | (4.5%) | | | 1 | 0 | 1 (100.0%) | 0 |

[1] P-VALUES ARE FROM CHISQ TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
NOTE:
ADVERSE EVENTS RELATED TO STUDY DRUG: RELATIONSHIP TO STUDY DRUG = ‘SUSPECT’ OR ‘PROBABLE’.
*, **, ***P-VALUE <=0.05, <=0.01, or <=0.001 RESPECTIVELY.

TABLE 52D
SELECTED ADVERSE EVENTS
SAFETY POPULATION, MALE PATIENTS

| ADVERSE | | TOTAL | NO. OF | | | NUMBER | | | |
|---|---|---|---|---|---|---|---|---|---|
| EVENT | | NO. OF | SUBJECTS | | | OF | SEVERITY [2] | | |
| (ENGLISH) | TREATMENT | SUBJECTS | W/EVENT | SOURCE | P-VALUE [1] | EVENTS | Mild | Moderate | Severe |
| DIZZINESS | A) PLACEBO | 19 | 1 (5.3%) | Treatment | 0.008** | 1 | 0 | 1 (100.0%) | 0 |
| (Exc. Vertigo) | B) MS 60 mg | 25 | 3 (12.0%) | A-D | 0.046* | 3 | 1 (33.3%) | 2 (66.7%) | 0 |
| | C) NTX 0.01 mg | 21 | 0 | A-E | 0.020* | 0 | 0 | 0 | 0 |
| | D) MS 60 mg/NTX | 32 | 9 (28.1%) | A-F | 0.032* | 10 | 4 (40.0%) | 5 (50.0%) | 1 (10.0%) |
| | 0.001 mg | | | | | | | | |
| | E) MS 60 mg/NTX | 23 | 8 (34.8%) | C-D | 0.007** | 9 | 5 (55.6%) | 4 (44.4%) | 0 |
| | 0.01 mg | | | | | | | | |
| | F) MS 60 mg/NTX | 22 | 7 (31.8%) | C-E | 0.002** | 9 | 4 (44.4%) | 4 (44.4%) | 1 (11.1%) |
| | 0.1 mg | | | | | | | | |
| | | | | C-F | 0.004** | | | | |
| NAUSEA | A) PLACEBO | 19 | 2 (10.5%) | Treatment | 0.001** | 2 | 1 (50.0%) | 1 (50.0%) | 0 |
| | B) MS 60 mg | 25 | 10 (40.0%) | A-B | 0.029* | 10 | 7 (70.0%) | 3 (30.0%) | 0 |
| | C) NTX 0.01 mg | 21 | 0 | A-D | 0.013* | 0 | 0 | 0 | 0 |
| | D) MS 60 mg/NTX | 32 | 14 (43.8%) | A-F | 0.014* | 15 | 5 (33.3%) | 7 (46.7%) | 3 (20.0%) |
| | 0.001 mg | | | | | | | | |
| | E) MS 60 mg/NTX | 23 | 6 (26.1%) | B-C | 0.001** | 6 | 2 (33.3%) | 2 (33.3%) | 2 (33.3%) |
| | 0.01 mg | | | | | | | | |
| | F) MS 60 mg/NTX | 22 | 10 (45.5%) | C-D | <0.001*** | 10 | 6 (60.0%) | 4 (40.0%) | 0 |
| | 0.1 mg | | | | | | | | |
| | | | | C-E | 0.011* | | | | |
| SOMNOLENCE | A) PLACEBO | 19 | 0 | Treatment | 0.209 | 0 | 0 | 0 | 0 |
| | B) MS 60 mg | 25 | 3 (12.0%) | C-F | 0.040* | 4 | 1 (25.0%) | 3 (75.0%) | 0 |
| | C) NTX 0.01 mg | 21 | 0 | | | 0 | 0 | 0 | 0 |
| | D) MS 60 mg/NTX | 32 | 5 (15.6%) | | | 6 | 4 (66.7%) | 2 (33.3%) | 0 |
| | 0.001 mg | | | | | | | | |
| | E) MS 60 mg/NTX | 23 | 3 (13.0%) | | | 3 | 1 (33.3%) | 2 (66.7%) | 0 |
| | 0.01 mg | | | | | | | | |
| | F) MS 60 mg/NTX | 22 | 4 (18.2%) | | | 4 | 1 (25.0%) | 3 (75.0%) | 0 |
| | 0.1 mg | | | | | | | | |
| VOMITING | A) PLACEBO | 19 | 1 (5.3%) | Treatment | <0.001*** | 1 | 0 | 0 | 1 (100.0%) |
| NOS | B) MS 60 mg | 25 | 9 (36.0%) | A-B | 0.015* | 9 | 3 (33.3%) | 2 (22.2%) | 4 (44.4%) |
| | C) NTX 0.01 mg | 21 | 0 | A-D | 0.010* | 0 | 0 | 0 | 0 |
| | D) MS 60 mg/NTX | 32 | 12 (37.5%) | A-E | 0.020* | 13 | 2 (15.4%) | 6 (46.2%) | 5 (38.5%) |
| | 0.001 mg | | | | | | | | |
| | E) MS 60 mg/NTX | 23 | 8 (34.8%) | A-F | 0.001** | 11 | 1 (9.1%) | 2 (18.2%) | 8 (72.7%) |
| | 0.01 mg | | | | | | | | |
| | F) MS 60 mg/NTX | 22 | 11 (50.0%) | B-C | 0.002** | 12 | 1 (8.3%) | 1 (8.3%) | 10 (83.3%) |
| | 0.1 mg | | | | | | | | |

[1] P-VALUES ARE FROM CHISQ TEST AND ARE PROVIDED FOR OVERALL TREATMENT EFFECT AND SIGNIFICANT PAIRWISE COMPARISONS ONLY.
[2] THE DENOMINATOR FOR THE PERCENTAGES IS THE TOTAL NUMBER OF EVENTS.
NOTE:
ADVERSE EVENTS RELATED TO STUDY DRUG: RELATIONSHIP TO STUDY DRUG = ‘SUSPECT’ OR ‘PROBABLE’.
*, **, ***P-VALUE <=0.05, <=0.01, or <=0.001 RESPECTIVELY.

### EXAMPLE 5

An additional clinical study, this one using hydrocodone with acetaminophen (instead of morphine) alone and in combination with naltrexone, was designed substantially the same as that described in Example 3, with the following, differences: (1) six treatment groups with four different doses of NTX (1.0 mg, 0.1 mg, 0.01 mg and 0.001 mg) in combination with hydrocodone 5 mg/acetaminophen 500 mg versus hydrocodone 5 mg/acetaminophen 500 mg (HC/APAP) alone, and versus placebo alone in subjects with moderate to severe pain in a postsurgical dental pain clinical study; (2) the primary efficacy variable was the categorical sum of pain intensity difference scores through 4 hours (SPID-4); and (3) the secondary efficacy variables were: 4, 6 and 8 hour total pain relief scores (TOTPAR-4, TOTPAR-6 and TOTPAR-8); categorical 6 and 8 hour sum of pain intensity difference scores (SPID-6 and SPID-8); categorical pain intensity difference (PD) scores through 8 hours; pain relief (PR) scores through 8 hours; peak categorical PD scores through 8 hours (PEAKPID); peak pain relief score through 8 hours (TOTPAR); time to onset of analgesia (i.e., at least a one category improvement in the pain intensity score); time to onset of meaningful pain relief; time to taking backup medication; percent of patients taking backup medication; and patient overall evaluation of study drug.

A total of 300 subjects were randomized; all 300 subjects were deemed evaluable (Table 53).

TABLE 53
Patients Enrollment and Evaluability

| | TREATMENTS | | | | | | |
|---|---|---|---|---|---|---|---|
| | | | W/NTX | W/NTX | W/NTX | W/NTX | |
| | Placebo | HC/APAP | 1 mg | 0.1 mg | 0.01 mg | 0.001 mg | TOTAL |
| Number of Patients | 50 | 50 | 50 | 50 | 50 | 50 | 300 |
| Patients Included in the | 50 (100%) | 50 (100%) | 50 (100%) | 50 (100%) | 50 (100%) | 50 (100%) | 300 (100%) |
| Safety Analyses | | | | | | | |
| Patients Excluded from | 0 (0%) | 0 (0%) | 0 (0%) | 0 (0%) | 0 (0%) | 0 (0%) | 0 (0%) |
| the Safety Analyses | | | | | | | |
| Patients Included in the | 50 (100%) | 50 (100%) | 50 (100%) | 50 (100%) | 50 (100%) | 50 (100%) | 300 (100%) |
| Efficacy Analyses | | | | | | | |
| Patients Excluded from | 0 (0%) | 0 (0%) | 0 (0%) | 0 (0%) | 0 (0%) | 0 (0%) | 0 (0%) |
| the Efficacy Analyses | | | | | | | |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***: P-VALUE <=0.05, <=0.01, or <=0.001 RESPECTIVELY.

The demographic and baseline characteristics were summarized by treatment groups for all 300 randomized patients which were all evaluable (Table 54). Demographic characteristics included age, race/ethnicity, sex, weight, height, medical history, teeth extracted (impacted and non-impacted), baseline pain intensity, and baseline visual analog scale.

Subjects ranged in age from 16 to 53 years; 79.0% were Caucasian and 63.0% were female. No adjustments in the analyses were made to take into account differences among treatment groups. These differences had little or no influence on pain assessments at baseline. The baseline pain intensity scores and visual analog scale scores were generally comparable across treatment groups (Tables 55A and 55B).

TABLE 54
Baseline Characteristics
Safety Patients

| | | | | W/NTX | W/NTX | | W/NTX | | P- |
|---|---|---|---|---|---|---|---|---|---|
| | | Placebo | HC/APAP | 1 mg | 0.1 mg | W/NTX 0.01 mg | 0.001 mg | TOTAL | Value |
| Number of Patients | | 50 | 50 | 50 | 50 | 50 | 50 | 300 | |
| Gender | Female | 28 (56%) | 34 (68%) | 31 (62%) | 35 (70%) | 31 (62%) | 30 (60%) | 189 (63%) | 0.716^{b} |
| (n, %) | Male | 22 (44%) | 16 (32%) | 19 (38%) | 15 (30%) | 19 (38%) | 20 (40%) | 111 (37%) | |
| Age | N | 50 | 50 | 50 | 50 | 50 | 50 | 300 | 0.199^{a} |
| (yrs) | Mean | 23.9 | 21.6 | 22.5 | 23.1 | 21.1 | 21.5 | 22.3 | |
| | SD | 7.8 | 4.5 | 6.0 | 7.2 | 4.4 | 6.8 | 6.3 | |
| | Median | 22.0 | 20.0 | 20.5 | 21.5 | 20.0 | 19.0 | 20.0 | |
| | Range | 16 to 46 | 16 to 35 | 16 to 41 | 16 to 53 | 16 to 35 | 16 to 48 | 16 to 53 | |
| Height | N | 50 | 50 | 50 | 50 | 50 | 50 | 300 | 0.823^{a} |
| (in) | Mean | 67.2 | 66.9 | 67.0 | 66.4 | 66.9 | 67.6 | 67.0 | |
| | SD | 4.4 | 3.7 | 3.9 | 4.2 | 4.3 | 4.2 | 4.1 | |
| | Median | 66.5 | 66.0 | 66.0 | 66.0 | 66.3 | 67.0 | 66.0 | |
| | Range | 60 to 76 | 61 to 75 | 61 to 78 | 61 to 79 | 61 to 77 | 61 to 79 | 60 to 79 | |
| Weight | N | 50 | 50 | 50 | 50 | 50 | 50 | 300 | 0.955^{a} |
| (lbs) | Mean | 159.4 | 152.5 | 156.4 | 154.9 | 155.3 | 156.3 | 155.8 | |
| | SD | 40.5 | 32.9 | 29.5 | 36.4 | 24.9 | 37.3 | 33.8 | |
| | Median | 155.5 | 149.5 | 154.5 | 144.5 | 155.5 | 150.0 | 150.5 | |
| | Range | 61 to 256 | 104 to 271 | 101 to 239 | 105 to 284 | 98 to 218 | 105 to 244 | 61 to 284 | |
| Ethnic | Caucasian | 34 (68%) | 40 (80%) | 42 (84%) | 42 (84%) | 38 (76%) | 41 (82%) | 237 (79%) | 0.362^{b} |
| Origin | Hispanic | 14 (28%) | 4 (8%) | 5 (10%) | 7 (14%) | 10 (20%) | 5 (10%) | 45 (15%) | |
| (n, %) | Black | 1 (2%) | 3 (6%) | 2 (4%) | 0 (0%) | 0 (0%) | 3 (6%) | 9 (3%) | |
| | Asian | 0 (0%) | 2 (4%) | 1 (2%) | 0 (0%) | 0 (0%) | 0 (0%) | 3 (1%) | |
| | Caucasian/Hispanic | 0 (0%) | 0 (0%) | 0 (0%) | 1 (2%) | 0 (0%) | 0 (0%) | 1 (<1%) | |
| | German/Arabic | 0 (0%) | 0 (0%) | 0 (0%) | 0 (0%) | 1 (2%) | 0 (0%) | 1 (<1%) | |
| | Lebanese | 0 (0%) | 1 (2%) | 0 (0%) | 0 (0%) | 0 (0%) | 0 (0%) | 1 (<1%) | |
| | Mexican/Korean | 0 (0%) | 0 (0%) | 0 (0%) | 0 (0%) | 1 (2%) | 0 (0%) | 1 (<1%) | |
| | Moroccan | 1 (2%) | 0 (0%) | 0 (0%) | 0 (0%) | 0 (0%) | 0 (0%) | 1 (<1%) | |
| | Mullato | 0 (0%) | 0 (0%) | 0 (0%) | 0 (0%) | 0 (0%) | 1 (2%) | 1 (<1%) | |

[1] P-VALUES ARE FROM TWO-WAY ANALYSIS OF VARIANCE AND ITS CONTRASTS WITH TREATMENT, SITE, AND TREATMENT BY SITE INTERACTIONS AS FACTORS.
*, **, ***: P-VALUE <=0.05, <=0.01, OR <=0.001 RESPECTIVELY

TABLE 55A
Summary of Baseline Pain Intensity Scores
(Safety Patients)

PAIN INTENSITY

| TREATMENT | MODERATE | SEVERE | P-Value |
|---|---|---|---|
| A) Placebo | 34 (68%) | 16 (32%) | 1.000^{b} |
| B) HC/APAP | 34 (68%) | 16 (32%) | |
| C) W/NTX 1 mg | 34 (68%) | 16 (32%) | |
| D) W/NTX 0.1 mg | 35 (70%) | 15 (30%) | |
| E) W/NTX 0.01 mg | 34 (68%) | 16 (32%) | |
| F) W/NTX 0.001 mg | 34 (68%) | 16 (32%) | |
| TOTAL | 205 (68%) | 95 (32%) | |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***: P-VALUE <=0.05, <=0.01, or <=0.001 RESPECTIVELY.

TABLE 55B
Summary of Baseline Visual Analog Scale (VAS) Scores
(Safety Patients)

| BASELINE VAS SCORE (0-100 mm Scale) | | | | | | P- |
|---|---|---|---|---|---|---|
| TREATMENT | N | MEAN | SD | MEDIAN | RANGE | Value |
| A) Placebo | 50 | 61.0 | 9.9 | 59.0 | 47 to 94 | 0.866^{a} |
| B) HC/APAP | 50 | 62.2 | 11.6 | 60.0 | 47 to 92 | |
| C) W/NTX | 50 | 61.0 | 8.5 | 60.0 | 47 to 83 | |
| 1 mg | | | | | | |
| D) W/NTX | 50 | 62.3 | 11.6 | 60.0 | 47 to 100 | |
| 0.1 mg | | | | | | |
| E) W/NTX | 50 | 63.3 | 9.4 | 60.0 | 48 to 89 | |
| 0.01 mg | | | | | | |
| F) W/NTX | 50 | 62.6 | 10.4 | 60.0 | 47 to 87 | |
| 0.001 mg | | | | | | |
| TOTAL | 300 | 62.1 | 10.2 | 60.0 | 47 to 100 | |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***: P-VALUE <=0.05, <=0.01, or <=0.001 RESPECTIVELY.

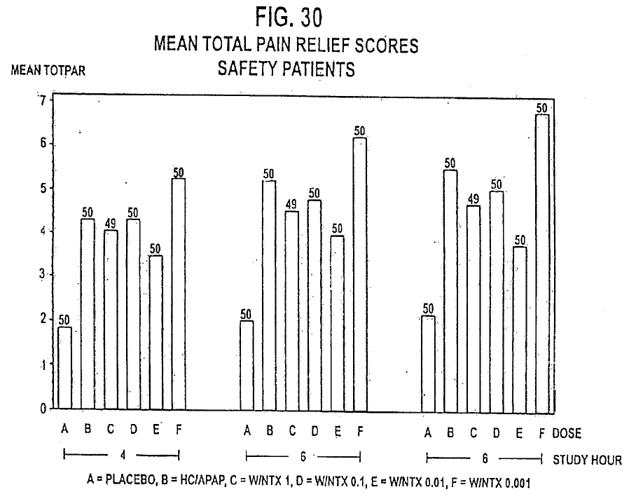

The TOTPAR results (4 hour, 6 hour, 8 hour) are summarized in Table 56 and the 4 hour TOTPAR scores are shown in FIG. 30. The placebo treatment group had the lowest mean TOTPAR scores. All 5 of the active treatment groups with HC/APAP alone or in combination with NTX exhibited mean TOTPAR scores that were numerically higher than placebo. The mean TOTPAR score for the 0.001 mg NTX combination treatment was higher than that for the HC/APAP alone treatment, whereas the other NTX combination treatment means were comparable to or lower than that for the HC/APAP alone treatment (FIG. 30).

TABLE 56
Efficacy Results - Means and Standard Deviations for TOTPARs
(Trapezoidal Method) (Safety Patients)

| TOTAL PAIN RELIEF SCORES | | | | | P-Value |
|---|---|---|---|---|---|
| TREATMENT | N | MEAN | SD | SOURCE | [1] |

TOTAL PAIN RELIEF SCORES (4 HOURS)

| | | | | | |
|---|---|---|---|---|---|
| A) Placebo | 50 | 1.83 | 2.54 | TRT | <0.001 |
| B) HC/APAP | 50 | 4.29 | 3.99 | A-B | <0.001 |
| C) W/NTX 1 mg | 49 | 4.04 | 3.82 | A-C | 0.003 |
| D) W/NTX 0.1 mg | 50 | 4.29 | 3.47 | A-D | <0.001 |
| E) W/NTX 0.01 mg | 50 | 3.47 | 3.64 | A-E | 0.025 |
| F) W/NTX 0.001 mg | 50 | 5.25 | 4.15 | A-F | <0.001 |
| | | | | B-C | 0.736 |
| | | | | B-D | 0.994 |
| | | | | B-E | 0.259 |
| | | | | B-F | 0.188 |

TOTAL PAIN RELIEF SCORES (6 HOURS)

| | | | | | |
|---|---|---|---|---|---|
| A) Placebo | 50 | 2.02 | 3.32 | TRT | 0.001 |
| B) HC/APAP | 50 | 5.21 | 5.70 | A-B | 0.001 |
| C) W/NTX 1 mg | 49 | 4.51 | 4.79 | A-C | 0.012 |
| D) W/NTX 0.1 mg | 50 | 4.77 | 4.47 | A-D | 0.005 |
| E) W/NTX 0.01 mg | 50 | 3.96 | 4.76 | A-E | 0.050 |
| F) W/NTX 0.001 mg | 50 | 6.19 | 6.01 | A-F | <0.001 |
| | | | | B-C | 0.480 |
| | | | | B-D | 0.659 |
| | | | | B-E | 0.204 |
| | | | | B-F | 0.320 |

TOTAL PAIN RELIEF SCORES (8 HOURS)

| | | | | | |
|---|---|---|---|---|---|
| A) Placebo | 50 | 2.17 | 4.14 | TRT | 0.002 |
| B) HC/APAP | 50 | 5.48 | 6.25 | A-B | 0.004 |
| C) W/NTX 1 mg | 49 | 4.68 | 5.38 | A-C | 0.027 |
| D) W/NTX 0.1 mg | 50 | 5.01 | 5.20 | A-D | 0.012 |
| E) W/NTX 0.01 mg | 49 | 3.74 | 4.58 | A-E | 0.164 |
| F) W/NTX 0.001 mg | 50 | 6.77 | 7.53 | A-F | <0.001 |
| | | | | B-C | 0.482 |
| | | | | B-D | 0.680 |
| | | | | B-E | 0.126 |
| | | | | B-F | 0.253 |

[1] P-VALUES ARE FROM TWO-WAY ANALYSIS OF VARIANCE AND ITS CONTRASTS WITH TREATMENT, SITE, AND TREATMENT BY SITE INTERACTION AS FACTORS.
*, **, ***: P-VALUE <=0.05, <=0.01, or <=0.001 RESPECTIVELY.

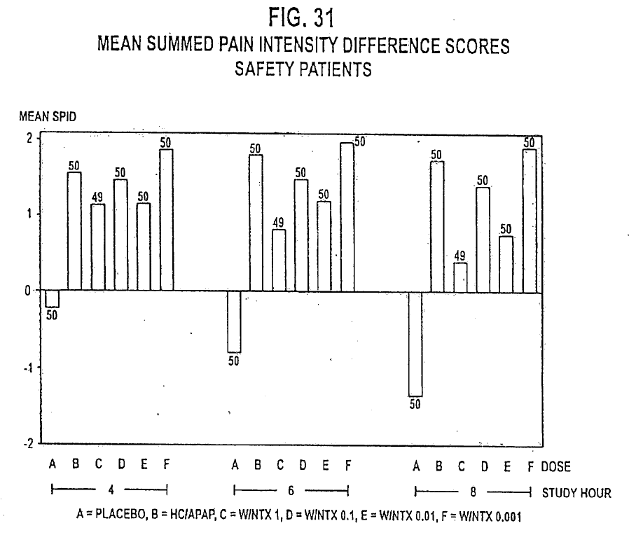

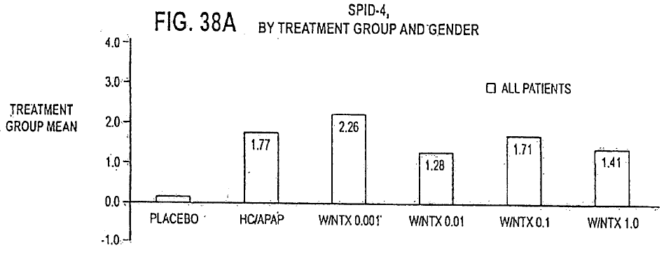

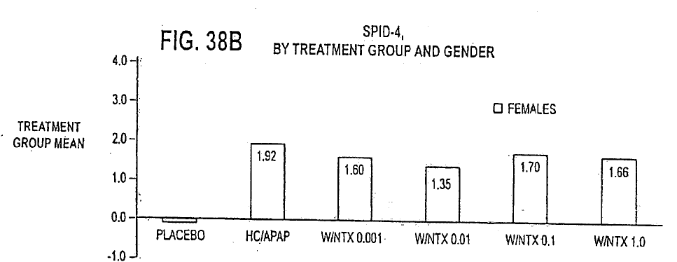

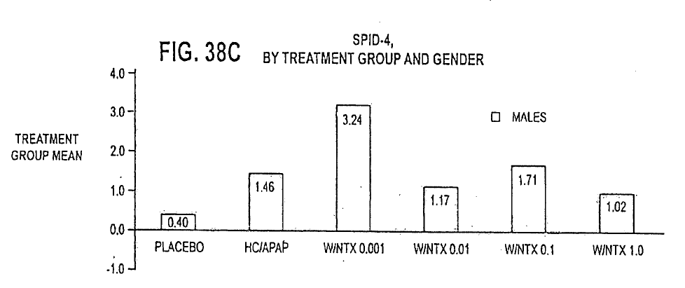

Table 57 summarizes the results of the 4, 6, and 8 hour SPID results (FIG. 31). The 4 hour results are also represented in FIG. 38A. The placebo treatment group had the lowest mean 4 hour SPID scores. All 5 of the active treatment groups with HC/APAP alone or in combination with NTX exhibited improved profiles in mean SPID relative to placebo. The mean 4 hour SPID score for the 0.001 mg NTX combination treatment was higher than that for the HC/APAP alone treatment, whereas the other NTX combination treatments were comparable to or lower than that for the HC/APAP alone treatment (FIG. 31 or 38A).

The patterns of the 6 hour and 8 hour SPID scores were similar to those at 4 hours.

TABLE 57
Efficacy Results - Means and Standard Deviations
for the SPIDS (Safety Patients)
Summary of Pin Intensity Differences (SPIDS)

| CATEGORICAL SPID SCORES | | | | | P-Value |
|---|---|---|---|---|---|
| TREATMENT | N | MEAN | SD | SOURCE | [1] |

CATEGORICAL SPID SCORES (4 HOURS)

| | | | | | |
|---|---|---|---|---|---|
| A) Placebo | 50 | −0.22 | 2.51 | TRT | 0.001 |
| B) HC/APAP | 50 | 1.55 | 2.42 | A-B | <0.001 |
| C) W/NTX 1 mg | 49 | 1.13 | 2.69 | A-C | 0.008 |
| D) W/NTX 0.1 mg | 50 | 1.46 | 2.07 | A-D | <0.001 |
| E) W/NTX 0.01 mg | 50 | 1.15 | 2.33 | A-E | 0.007 |
| F) W/NTX 0.001 mg | 50 | 1.87 | 2.89 | A-F | <0.001 |
| | | | | B-C | 0.406 |
| | | | | B-D | 0.852 |
| | | | | B-E | 0.422 |
| | | | | B-F | 0.529 |

CATEGORICAL SPID SCORES (6 HOURS)

| | | | | | |
|---|---|---|---|---|---|
| A) Placebo | 50 | −0.79 | 3.68 | TRT | 0.001 |
| B) HC/APAP | 50 | 1.80 | 3.43 | A-B | <0.001 |
| C) W/NTX 1 mg | 49 | 0.81 | 3.53 | A-C | 0.025 |
| D) W/NTX 0.1 mg | 50 | 1.47 | 2.84 | A-D | 0.001 |
| E) W/NTX 0.01 mg | 50 | 1.19 | 3.34 | A-E | 0.005 |
| F) W/NTX 0.001 mg | 50 | 1.98 | 4.17 | A-F | <0.001 |
| | | | | B-C | 0.164 |
| | | | | B-D | 0.643 |
| | | | | B-E | 0.386 |
| | | | | B-F | 0.804 |

CATEGORICAL SPID SCORES (8 HOURS)

| | | | | | |
|---|---|---|---|---|---|
| A) Placebo | 50 | −1.36 | 4.92 | TRT | 0.002 |
| B) HC/APAP | 50 | 1.73 | 3.92 | A-B | <0.001 |
| C) W/NTX 1 mg | 49 | 0.38 | 4.34 | A-C | 0.045 |
| D) W/NTX 0.1 mg | 50 | 1.38 | 3.55 | A-D | 0.002 |
| E) W/NTX 0.01 mg | 49 | 0.74 | 3.40 | A-E | 0.016 |
| F) W/NTX 0.001 mg | 50 | 1.91 | 5.27 | A-F | <0.001 |
| | | | | B-C | 0.119 |
| | | | | B-D | 0.683 |
| | | | | B-E | 0.250 |
| | | | | B-F | 0.839 |

MEANS GIVEN ARE LEAST SQUARE MEANS.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNIFICANT).

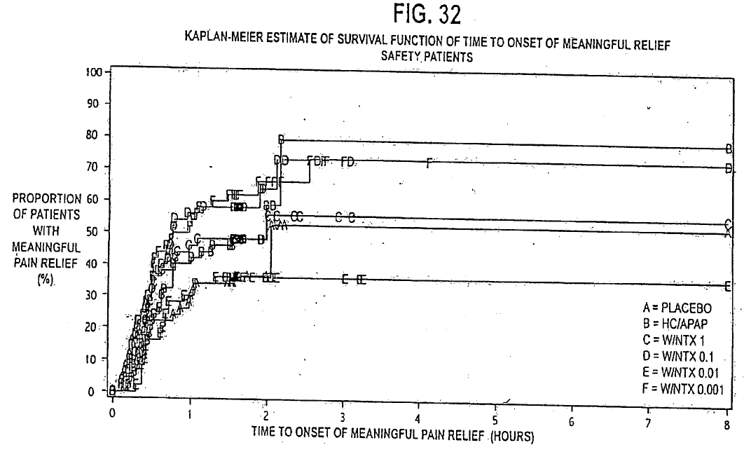

FIG. 32 is a visual presentation of the summary and analysis of time to onset of meaningful pain relief-presented in Table 58A. The median time to onset of meaningful pain relief was shortest in the 0.001 mg NTX (lowest-dose) combination treatment group. The placebo and the 0.01 mg NTX combination treatment groups had the lowest number of subjects who reached meaningful pain relief.

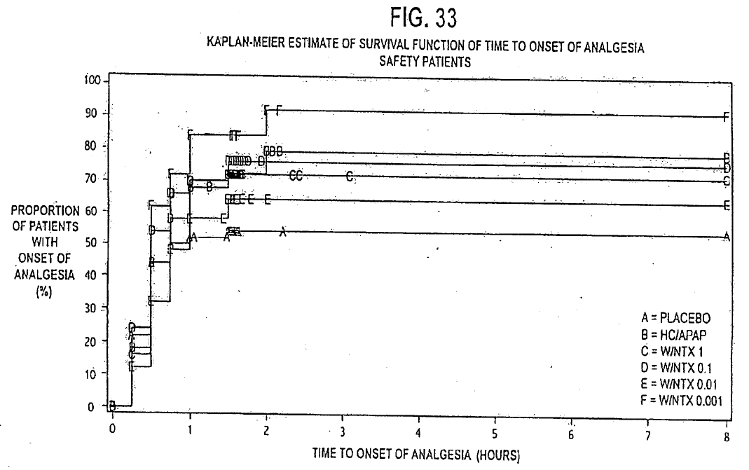

FIG. 33 is a visual presentation of the summary and analysis of time to onset of analgesia presented in Table 58B. The median time to onset of analgesia was shortest in the 0.001 mg NTX and 0.1 mg NTX combination treatment groups. The placebo treatment group had the lower number of subjects who reached analgesia.

TABLE 58A
Efficacy Results - Results of Time to Onset of Relief
(Safety Patients)
TIME TO ONSET OF RELIEF (hours)

| | | | 95% INTERVAL | | | | |
|---|---|---|---|---|---|---|---|
| | | MEDIAN | (hh:mm) | | | P-Value | P-Value |
| | NUMBER OF | TIME | LOWER | UPPER | | vs. | vs. |
| TREATMENT | PATIENTS | (hh:mm) | LIMIT | LIMIT | P-Value | Placebo | HC/APAP |
| A) Placebo | 50 | >8.0 | 2.1 | >8.0 | 0.008 | | |
| B) HC/APAP | 50 | 2.0 | 0.8 | >8.0 | | 0.230 | |
| C) W/NTX 1 mg | 50 | >8.0 | 0.8 | >8.0 | | 0.347 | 0.891 |
| D) W/NTX 0.1 mg | 50 | 0.8 | 0.6 | >8.0 | | 0.019 | 0.199 |
| E) W/NTX 0.01 mg | 50 | >8.0 | 8.0 | >8.0 | | 0.619 | 0.087 |
| F) W/NTX 0.001 mg | 50 | 0.8 | 0.5 | 1.9 | | 0.010 | 0.122 |
| TOTAL | 300 | >8.0 | 1.1 | >8.0 | | | |

P-VALUES FOR TIME TO EVENT ARE FROM THE LOG RANK TEST.
P-VALUES FOR PERCENT OF PATIENTS WITH EVENT ARE FROM THE LIKELIHOOD - RATIO CHI-SQUARE TEST.

TABLE 58B
Efficacy Results - Results of Analgesia
(Safety Patients)
TIME TO ONSET OF ANALGESIA (hours)

| | | | 95% INTERVAL | | | | |
|---|---|---|---|---|---|---|---|
| | | MEDIAN | (hh:mm) | | | P-Value | P-Value |
| | NUMBER OF | TIME | LOWER | UPPER | | vs. | vs. |
| TREATMENT | PATIENTS | (hh:mm) | LIMIT | LIMIT | P-Value | Placebo | HC/APAP |
| A) Placebo | 50 | 0.8 | 0.5 | >8.0 | 0.058 | | |
| B) HC/APAP | 50 | 0.8 | 0.5 | 1.0 | | 0.178 | |
| C) W/NTX 1 mg | 50 | 0.8 | 0.5 | 0.8 | | 0.311 | 0.830 |
| D) W/NTX 0.1 mg | 50 | 0.5 | 0.5 | 0.8 | | 0.088 | 0.618 |
| E) W/NTX 0.01 mg | 50 | 1.0 | 0.8 | >8.0 | | 0.818 | 0.216 |
| F) W/NTX 0.001 mg | 50 | 0.5 | 0.5 | 0.8 | | 0.012 | 0.145 |
| TOTAL | 300 | 0.8 | 0.5 | 0.8 | | | |

P-VALUES FOR TIME TO EVENT ARE FROM THE LOG RANK TEST.
P-VALUES FOR PERCENT OF PATIENTS WITH EVENT ARE FROM THE LIKELIHOOD - RATIO CHI-SQUARE TEST.

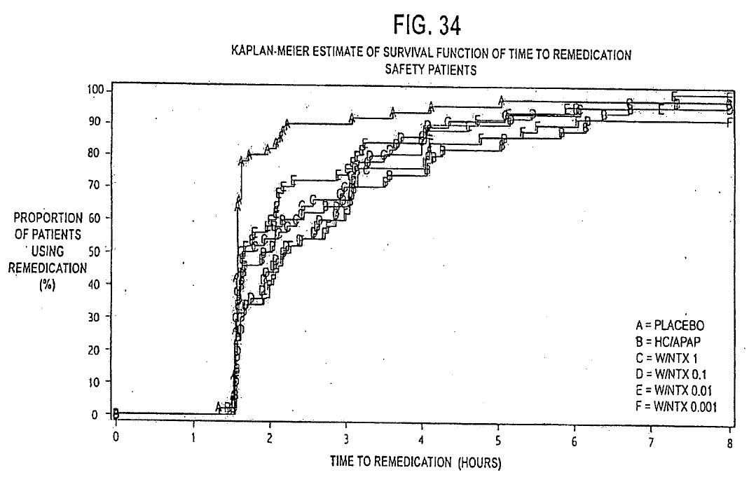

Table 59 summarizes the results of the time to remedication (see also FIG. 34). The placebo and the 1.0 mg NTX combination treatment groups had the shortest median time to remedication and the 0.1 mg NTX and the 0.001 NTX combination treatment groups had the longest median time to remedication.

Table 60 summarizes the results of the percent of patients remedicating. The percentage of patients remedicating was comparable across all treatment groups, except that the 0.001 mg NTX combination group had a lower percentage of patients remedicating.

TABLE 59
Efficacy Results - Time to Rescue Medication
(Safety Patients)
TIME TO REMEDICATION (hours)

| | | | 95% INTERVAL | | | | |
|---|---|---|---|---|---|---|---|
| | NUMBER | MEDIAN | (hh:mm) | | P-Value | P-Value | |
| | OF | TIME | LOWER | UPPER | vs. | vs. | |
| TREATMENT | PATIENTS | (hh:mm) | LIMIT | LIMIT | Placebo | HC/APAP | P-Value |
| A) Placebo | 50 | 1.6 | 1.6 | 1.6 | | | <0.001 |
| B) HC/APAP | 50 | 1.9 | 1.6 | 2.7 | <0.001 | | |
| C) W/NTX 1 mg | 50 | 1.6 | 1.6 | 2.4 | 0.008 | 0.346 | |
| D) W/NTX 0.1 mg | 50 | 2.2 | 1.9 | 2.9 | <0.001 | 0.749 | |
| E) W/NTX 0.01 mg | 50 | 1.7 | 1.6 | 2.1 | 0.017 | 0.208 | |
| F) W/NTX 0.001 mg | 50 | 2.2 | 2.0 | 3.1 | <0.001 | 0.587 | |
| TOTAL | 300 | 1.8 | 1.6 | 2.1 | | | |

NOTE:
MEDIAN TIME AND ITS CONFIDENCE INTERVAL ARE ESTIMATED USING KAPLAN-MEIER METHOD. LOG-RANK AND WILCOXON TESTS ARE USED TO TEST THE EQUALITY OF KAPLAN-MEIER SURVIVAL FUNCTIONS OVER DIFFERENT TREATMENT GROUPS.

TABLE 60
Efficacy Results Percent of Patients Remedicating
(Safety Patients)
PATIENTS REMEDICATING

| | | | P-Value | P-Value | |
|---|---|---|---|---|---|
| | | | vs. | vs. HC/ | P- |
| TREATMENT | YES | NO | Placebo | APAP | VALUE |
| A) Placebo | 49 (98%) | 1 (2%) | | | 0.699 |
| B) HC/APAP | 49 (98%) | 1 (2%) | 1.000 | | |
| C) W/NTX 1 mg | 48 (96%) | 2 (4%) | 1.000 | 1.000 | |
| D) W/NTX | 48 (96%) | 2 (4%) | 1.000 | 1.000 | |
| 0.1 mg | | | | | |
| E) W/NTX | 49 (98%) | 1 (2%) | 1.000 | 1.000 | |
| 0.01 mg | | | | | |
| F) W/NTX | 46 (92%) | 4 (8%) | 0.362 | 0.362 | |
| 0.001 mg | | | | | |
| TOTAL | 289 (96%) | 11 (4%) | | | |

P-VALUES FOR TIME TO EVENT ARE FROM THE LOG RANK TEST.
P-VALUES FOR PERCENT OF PATIENTS WITH EVENT ARE FROM THE LIKELIHOOD - RATIO CHI-SQUARE TEST.

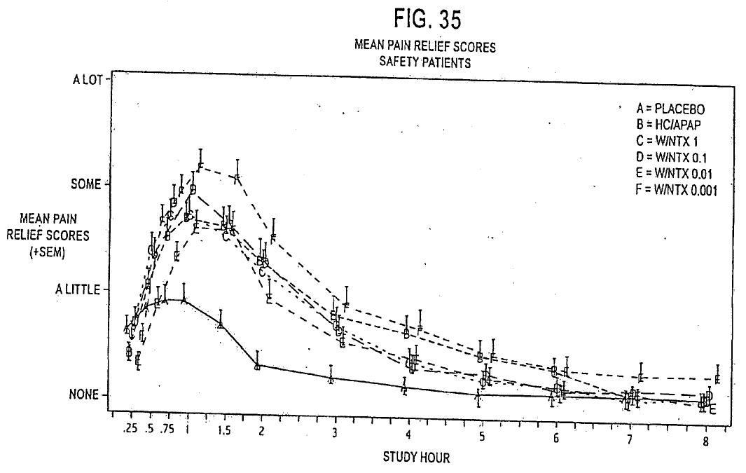

FIG. 35 is a visual presentation of the mean pain relief scores presented in Table 61. The mean pain relief score for the placebo treatment group was less than those for the active treatment groups (HC/APAP alone or in combination with NTX). There was separation between placebo and the active treatment groups from 1 hour to hours of the 8 hour study period. Highest pain relief scores were observed for the 0.001 mg NTX combination group (FIG. 35).

TABLE 61
Efficacy Results - Means and Standard Deviations for
the Pain Relief Scores (Safety Patients)

| PAIN RELIEF SCORE (PR) | | | | | |
|---|---|---|---|---|---|
| TREATMENT | N | MEAN | SD | SOURCE | P-VALUE |
| 15 MINUTES | | | | | |
| A) Placebo | 50 | 0.64 | 0.88 | TRT | 0.214 |
| B) HC/APAP | 50 | 0.42 | 0.64 | A-B | 0.174 |
| C) W/NTX 1 | 50 | 0.58 | 0.88 | A-C | 0.711 |
| D) W/NTX 0.1 | 50 | 0.70 | 1.04 | A-D | 0.711 |
| E) W/NTX 0.01 | 50 | 0.34 | 0.59 | A-E | 0.064 |
| F) W/NTX 0.001 | 50 | 0.58 | 0.73 | A-F | 0.711 |
| | | | | B-C | 0.323 |
| | | | | B-D | 0.084 |
| | | | | B-E | 0.621 |
| | | | | B-F | 0.323 |
| 30 MINUTES | | | | | |
| A) Placebo | 50 | 0.84 | 1.04 | TRT | 0.001 |
| B) HC/APAP | 50 | 1.05 | 1.07 | A-B | 0.337 |
| C) W/NTX 1 | 50 | 1.38 | 1.19 | A-C | 0.016 |
| D) W/NTX 0.1 | 50 | 1.34 | 1.12 | A-D | 0.024 |
| E) W/NTX 0.01 | 50 | 0.88 | 1.10 | A-E | 0.857 |
| F) W/NTX 0.001 | 50 | 1.66 | 1.14 | A-F | <0.001 |
| | | | | B-C | 0.143 |
| | | | | B-D | 0.194 |
| | | | | B-E | 0.435 |
| | | | | B-F | 0.007 |
| 45 MINUTES | | | | | |
| A) Placebo | 50 | 0.92 | 1.01 | TRT | <0.001 |
| B) HC/APAP | 50 | 1.52 | 1.11 | A-B | 0.007 |
| C) W/NTX 1 | 50 | 1.71 | 1.14 | A-C | <0.001 |
| D) W/NTX 0.1 | 50 | 1.84 | 1.18 | A-D | <0.001 |
| E) W/NTX 0.01 | 50 | 1.32 | 1.00 | A-E | 0.069 |
| F) W/NTX 0.001 | 50 | 1.95 | 1.13 | A-F | <0.001 |
| | | | | B-C | 0.381 |
| | | | | B-D | 0.148 |
| | | | | B-E | 0.363 |
| | | | | B-F | 0.053 |
| 1 HOUR | | | | | |
| A) Placebo | 50 | 0.92 | 1.14 | TRT | <0.001 |
| B) HC/APAP | 50 | 1.69 | 1.06 | A-B | 0.002 |
| C) W/NTX 1 | 50 | 1.72 | 1.29 | A-C | 0.001 |
| D) W/NTX 0.1 | 50 | 1.96 | 1.27 | A-D | <0.001 |
| E) W/NTX 0.01 | 50 | 1.59 | 1.29 | A-E | 0.006 |
| F) W/NTX 0.001 | 50 | 2.18 | 1.22 | A-F | <0.001 |
| | | | | B-C | 0.913 |
| | | | | B-D | 0.276 |
| | | | | B-E | 0.671 |
| | | | | B-F | 0.046 |
| 1.5 HOURS | | | | | |
| A) Placebo | 50 | 0.70 | 0.95 | TRT | <0.001 |
| B) HC/APAP | 50 | 1.62 | 1.29 | A-B | <0.001 |
| C) W/NTX 1 | 50 | 1.52 | 1.40 | A-C | 0.001 |
| D) W/NTX 0.1 | 50 | 1.64 | 1.27 | A-D | <0.001 |
| E) W/NTX 0.01 | 50 | 1.58 | 1.31 | A-E | <0.001 |
| F) W/NTX 0.001 | 50 | 2.08 | 1.29 | A-F | <0.001 |
| | | | | B-C | 0.692 |
| | | | | B-D | 0.937 |
| | | | | B-E | 0.874 |
| | | | | B-F | 0.069 |
| 2 HOURS | | | | | |
| A) Placebo | 50 | 0.32 | 0.91 | TRT | <0.001 |
| B) HC/APAP | 50 | 1.30 | 1.50 | A-B | <0.001 |
| C) W/NTX 1 | 50 | 1.19 | 1.52 | A-C | 0.002 |
| D) W/NTX 0.1 | 50 | 1.28 | 1.37 | A-D | <0.001 |
| E) W/NTX 0.01 | 50 | 0.94 | 1.35 | A-E | 0.024 |
| F) W/NTX 0.001 | 50 | 1.50 | 1.45 | A-F | <0.001 |
| | | | | B-C | 0.699 |
| | | | | B-D | 0.942 |
| | | | | B-E | 0.188 |
| | | | | B-F | 0.464 |
| 3 HOURS | | | | | |
| A) Placebo | 50 | 0.22 | 0.79 | TRT | 0.076 |
| B) HC/APAP | 50 | 0.80 | 1.28 | A-B | 0.013 |
| C) W/NTX 1 | 50 | 0.70 | 1.23 | A-C | 0.039 |
| D) W/NTX 0.1 | 50 | 0.65 | 1.08 | A-D | 0.066 |
| E) W/NTX 0.01 | 50 | 0.54 | 1.13 | A-E | 0.170 |
| F) W/NTX 0.001 | 50 | 0.88 | 1.38 | A-F | 0.005 |
| | | | | B-C | 0.678 |
| | | | | B-D | 0.517 |
| | | | | B-E | 0.265 |
| | | | | B-F | 0.731 |
| 4 HOURS | | | | | |
| A) Placebo | 50 | 0.14 | 0.70 | TRT | 0.098 |
| B) HC/APAP | 50 | 0.64 | 1.24 | A-B | 0.018 |
| C) W/NTX 1 | 49 | 0.36 | 0.97 | A-C | 0.291 |
| D) W/NTX 0.1 | 50 | 0.32 | 0.91 | A-D | 0.393 |
| E) W/NTX 0.01 | 50 | 0.40 | 0.99 | A-E | 0.217 |
| F) W/NTX 0.001 | 50 | 0.68 | 1.36 | A-F | 0.011 |
| | | | | B-C | 0.193 |
| | | | | B-D | 0.129 |
| | | | | B-E | 0.255 |
| | | | | B-F | 0.849 |
| 5 HOURS | | | | | |
| A) Placebo | 50 | 0.08 | 0.44 | TRT | 0.253 |
| B) HC/APAP | 50 | 0.44 | 1.07 | A-B | 0.040 |
| C) W/NTX 1 | 49 | 0.20 | 0.76 | A-C | 0.479 |
| D) W/NTX 0.1 | 50 | 0.26 | 0.80 | A-D | 0.303 |
| E) W/NTX 0.01 | 50 | 0.22 | 0.82 | A-E | 0.422 |
| F) W/NTX 0.001 | 50 | 0.44 | 1.15 | A-F | 0.040 |
| | | | | B-C | 0.179 |
| | | | | B-D | 0.303 |
| | | | | B-E | 0.208 |
| | | | | B-F | 1.000 |
| 6 HOURS | | | | | |
| A) Placebo | 50 | 0.08 | 0.57 | TRT | 0.445 |
| B) HC/APAP | 50 | 0.32 | 0.89 | A-B | 0.111 |
| C) W/NTX 1 | 49 | 0.16 | 0.72 | A-C | 0.582 |
| D) W/NTX 0.1 | 50 | 0.12 | 0.59 | A-D | 0.790 |
| E) W/NTX 0.01 | 50 | 0.14 | 0.64 | A-E | 0.690 |
| F) W/NTX 0.001 | 50 | 0.32 | 1.00 | A-F | 0.111 |
| | | | | B-C | 0.300 |
| | | | | B-D | 0.184 |
| | | | | B-E | 0.232 |
| | | | | B-F | 1.000 |
| 7 HOURS | | | | | |
| A) Placebo | 50 | 0.08 | 0.57 | TRT | 0.492 |
| B) HC/APAP | 50 | 0.08 | 0.40 | A-B | 1.000 |
| C) W/NTX 1 | 49 | 0.06 | 0.43 | A-C | 0.878 |
| D) W/NTX 0.1 | 50 | 0.12 | 0.59 | A-D | 0.742 |
| E) W/NTX 0.01 | 50 | 0.10 | 0.51 | A-E | 0.869 |
| F) W/NTX 0.001 | 50 | 0.28 | 0.97 | A-F | 0.101 |
| | | | | B-C | 0.878 |
| | | | | B-D | 0.742 |
| | | | | B-E | 0.869 |
| | | | | B-F | 0.101 |
| 8 HOURS | | | | | |
| A) Placebo | 50 | 0.06 | 0.42 | TRT | 0.179 |
| B) HC/APAP | 50 | 0.06 | 0.42 | A-B | 1.000 |
| C) W/NTX 1 | 49 | 0.06 | 0.43 | A-C | 0.991 |
| D) W/NTX 0.1 | 50 | 0.12 | 0.59 | A-D | 0.589 |
| E) W/NTX 0.01 | 49 | 0.00 | 0.00 | A-E | 0.591 |
| F) W/NTX 0.001 | 50 | 0.28 | 0.97 | A-F | 0.048 |
| | | | | B-C | 0.991 |
| | | | | B-D | 0.589 |
| | | | | B-E | 0.591 |
| | | | | B-F | 0.048 |

MEANS GIVEN ARE LEAST SQUARE MEANS.
THE PAIN RELIEF SCALE WAS: 0 = NONE, 1 = A LITTLE, 2 = SOME, 3 = A LOT, AND 4 = COMPLETE.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNIFICANT).

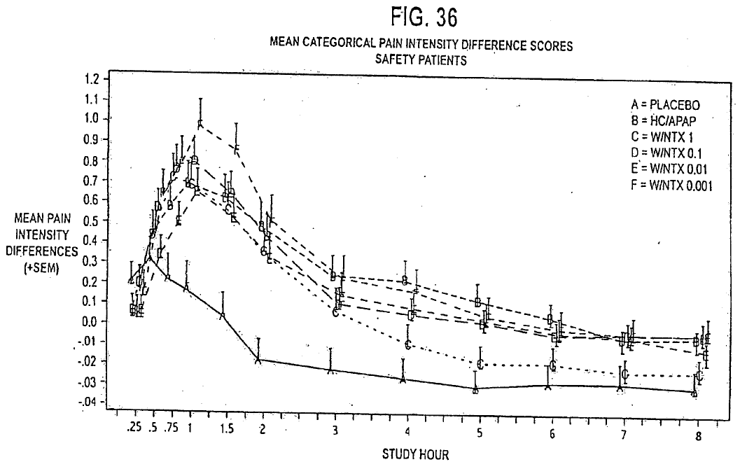

The mean categorical pain intensity difference (PID) scores are presented in Table 62 and FIG. 36. The mean PID scores for placebo treatment groups decreased over the first 2 hours and then were generally flat, while the mean PID scores first increase, then decreased over time for the active treatment groups (HC/APAP alone or in combination with NTX). The hourly mean scores for the HC/APAP alone and the HC/APAP NTX combination treatment groups were higher than the mean PID scores for the placebo group at each hourly assessment time from 1-8 hours. Highest pain relief as measured by mean PID scores was observed for the 0.001 NTX combination treatment group.

TABLE 62
Efficacy Results - Means and Standard Deviations
or the Categorical PID Scores
(Safety Patients)

| CATEGORICAL PID SCORES | | | | | |
|---|---|---|---|---|---|
| TREATMENT | N | MEAN | SD | SOURCE | P-Value |
| 15 MINUTES | | | | | |
| A) Placebo | 50 | 0.21 | 0.61 | TRT | 0.542 |
| B) HC/APAP | 50 | 0.06 | 0.55 | A-B | 0.187 |
| C) W/NTX 1 mg | 50 | 0.06 | 0.51 | A-C | 0.187 |
| D) W/NTX 0.1 mg | 50 | 0.20 | 0.57 | A-D | 0.930 |
| E) W/NTX 0.01 mg | 50 | 0.06 | 0.51 | A-E | 0.187 |
| F) W/NTX 0.001 mg | 50 | 0.15 | 0.64 | A-F | 0.597 |
| | | | | B-C | 1.000 |
| | | | | B-D | 0.218 |
| | | | | B-E | 1.000 |
| | | | | B-F | 0.428 |
| 30 MINUTES | | | | | |
| A) Placebo | 50 | 0.32 | 0.74 | TRT | 0.208 |
| B) HC/APAP | 50 | 0.44 | 0.79 | A-B | 0.420 |
| C) W/NTX 1 mg | 50 | 0.48 | 0.81 | A-C | 0.283 |
| D) W/NTX 0.1 mg | 50 | 0.57 | 0.64 | A-D | 0.089 |
| E) W/NTX 0.01 mg | 50 | 0.34 | 0.63 | A-E | 0.893 |
| F) W/NTX 0.001 mg | 50 | 0.64 | 0.83 | A-F | 0.032 |
| | | | | B-C | 0.788 |
| | | | | B-D | 0.370 |
| | | | | B-E | 0.502 |
| | | | | B-F | 0.180 |
| 45 MINUTES | | | | | |
| A) Placebo | 50 | 0.22 | 0.86 | TRT | 0.003 |
| B) HC/APAP | 50 | 0.58 | 0.76 | A-B | 0.023 |
| C) W/NTX 1 mg | 50 | 0.72 | 0.81 | A-C | 0.002 |
| D) W/NTX 0.1 mg | 50 | 0.76 | 0.77 | A-D | <0.001 |
| E) W/NTX 0.01 mg | 50 | 0.50 | 0.68 | A-E | 0.077 |
| F) W/NTX 0.001 mg | 50 | 0.78 | 0.84 | A-F | <0.001 |
| | | | | B-C | 0.376 |
| | | | | B-D | 0.255 |
| | | | | B-E | 0.613 |
| | | | | B-F | 0.206 |
| 1 HOUR | | | | | |
| A) Placebo | 50 | 0.17 | 0.99 | TRT | <0.001 |
| B) HC/APAP | 50 | 0.69 | 0.76 | A-B | 0.003 |
| C) W/NTX 1 mg | 50 | 0.69 | 0.90 | A-C | 0.003 |
| D) W/NTX 0.1 mg | 50 | 0.80 | 0.78 | A-D | <0.001 |
| E) W/NTX 0.01 mg | 50 | 0.65 | 0.80 | A-E | 0.006 |
| F) W/NTX 0.001 mg | 50 | 0.98 | 0.94 | A-F | <0.001 |
| | | | | B-C | 0.966 |
| | | | | B-D | 0.538 |
| | | | | B-E | 0.803 |
| | | | | B-F | 0.099 |
| 1.5 HOURS | | | | | |
| A) Placebo | 50 | 0.04 | 0.81 | TRT | <0.001 |
| B) HC/APAP | 50 | 0.62 | 0.83 | A-B | <0.001 |
| C) W/NTX 1 mg | 50 | 0.56 | 0.97 | A-C | 0.003 |
| D) W/NTX 0.1 mg | 50 | 0.64 | 0.78 | A-D | <0.001 |
| E) W/NTX 0.01 mg | 50 | 0.52 | 0.81 | A-E | 0.005 |
| F) W/NTX 0.001 mg | 50 | 0.86 | 0.93 | A-F | <0.001 |
| | | | | B-C | 0.727 |
| | | | | B-D | 0.907 |
| | | | | B-E | 0.560 |
| | | | | B-F | 0.163 |
| 2 HOURS | | | | | |
| A) Placebo | 50 | −0.18 | 0.77 | TRT | <0.001 |
| B) HC/APAP | 50 | 0.48 | 0.86 | A-B | <0.001 |
| C) W/NTX 1 mg | 50 | 0.35 | 1.01 | A-C | 0.002 |
| D) W/NTX 0.1 mg | 50 | 0.43 | 0.79 | A-D | <0.001 |
| E) W/NTX 0.01 mg | 50 | 0.32 | 0.77 | A-E | 0.004 |
| F) W/NTX 0.001 mg | 50 | 0.50 | 0.95 | A-F | <0.001 |
| | | | | B-C | 0.468 |
| | | | | B-D | 0.787 |
| | | | | B-E | 0.356 |
| | | | | B-F | 0.908 |
| 3 HOURS | | | | | |
| A) Placebo | 50 | −0.22 | 0.74 | TRT | 0.035 |
| B) HC/APAP | 50 | 0.24 | 0.72 | A-B | 0.003 |
| C) W/NTX 1 mg | 50 | 0.06 | 0.86 | A-C | 0.062 |
| D) W/NTX 0.1 mg | 50 | 0.10 | 0.59 | A-D | 0.034 |
| E) W/NTX 0.01 mg | 50 | 0.14 | 0.73 | A-E | 0.018 |
| F) W/NTX 0.001 mg | 50 | 0.22 | 0.86 | A-F | 0.004 |
| | | | | B-C | 0.242 |
| | | | | B-D | 0.363 |
| | | | | B-E | 0.508 |
| | | | | B-F | 0.895 |
| 4 HOURS | | | | | |
| A) Placebo | 50 | −0.26 | 0.69 | TRT | 0.008 |
| B) HC/APAP | 50 | 0.22 | 0.71 | A-B | <0.001 |
| C) W/NTX 1 mg | 49 | −0.09 | 0.68 | A-C | 0.227 |
| D) W/NTX 0.1 mg | 50 | 0.05 | 0.55 | A-D | 0.025 |
| E) W/NTX 0.01 mg | 50 | 0.08 | 0.67 | A-E | 0.015 |
| F) W/NTX 0.001 mg | 50 | 0.16 | 0.84 | A-F | 0.003 |
| | | | | B-C | 0.027 |
| | | | | B-D | 0.231 |
| | | | | B-E | 0.315 |
| | | | | B-F | 0.666 |
| 5 HOURS | | | | | |
| A) Placebo | 50 | −0.30 | 0.58 | TRT | 0.006 |
| B) HC/APAP | 50 | 0.12 | 0.63 | A-B | <0.001 |
| C) W/NTX 1 mg | 49 | −0.18 | 0.57 | A-C | 0.344 |
| D) W/NTX 0.1 mg | 50 | 0.01 | 0.48 | A-D | 0.011 |
| E) W/NTX 0.01 mg | 50 | 0.02 | 0.65 | A-E | 0.009 |
| F) W/NTX 0.001 mg | 50 | 0.04 | 0.73 | A-F | 0.006 |
| | | | | B-C | 0.014 |
| | | | | B-D | 0.382 |
| | | | | B-E | 0.413 |
| | | | | B-F | 0.513 |
| 6 HOURS | | | | | |
| A) Placebo | 50 | −0.28 | 0.67 | TRT | 0.064 |
| B) HC/APAP | 50 | 0.04 | 0.49 | A-B | 0.006 |
| C) W/NTX 1 mg | 49 | −0.18 | 0.57 | A-C | 0.409 |
| D) W/NTX 0.1 mg | 50 | −0.05 | 0.45 | A-D | 0.045 |
| E) W/NTX 0.01 mg | 50 | −0.04 | 0.57 | A-E | 0.039 |
| F) W/NTX 0.001 mg | 50 | −0.02 | 0.68 | A-F | 0.026 |
| | | | | B-C | 0.056 |
| | | | | B-D | 0.454 |
| | | | | B-E | 0.490 |
| | | | | B-F | 0.605 |
| 7 HOURS | | | | | |
| A) Placebo | 50 | −0.28 | 0.67 | TRT | 0.063 |
| B) HC/APAP | 50 | −0.06 | 0.31 | A-B | 0.032 |
| C) W/NTX 1 mg | 49 | −0.22 | 0.47 | A-C | 0.589 |
| D) W/NTX 0.1 mg | 50 | −0.05 | 0.45 | A-D | 0.023 |
| E) W/NTX 0.01 mg | 50 | −0.06 | 0.47 | A-E | 0.032 |
| F) W/NTX 0.001 mg | 50 | −0.04 | 0.60 | A-F | 0.019 |
| | | | | B-C | 0.110 |
| | | | | B-D | 0.898 |
| | | | | B-E | 1.000 |
| | | | | B-F | 0.845 |
| 8 HOURS | | | | | |
| A) Placebo | 50 | −0.30 | 0.58 | TRT | 0.026 |
| B) HC/APAP | 50 | −0.06 | 0.31 | A-B | 0.012 |
| C) W/NTX 1 mg | 49 | −0.22 | 0.47 | A-C | 0.427 |
| D) W/NTX 0.1 mg | 50 | −0.05 | 0.45 | A-D | 0.008 |
| E) W/NTX 0.01 mg | 49 | −0.12 | 0.33 | A-E | 0.062 |
| F) W/NTX 0.001 mg | 50 | −0.04 | 0.60 | A-F | 0.006 |
| | | | | B-C | 0.084 |
| | | | | B-D | 0.890 |
| | | | | B-E | 0.511 |
| | | | | B-F | 0.832 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNIFICANT).

Tables 63A and 63B present the mean peak (maximum) pain relief (MAXPAR) and mean peak pain intensity difference (PEAKPID) scores, respectively. The mean MAXPAR scores presented in Table 63A varied among treatment groups. The mean MAXPAR score was highest for the 0.001 mg NTX combination treatment group compared to all other groups. The mean scores for the other NTX combination treatment groups were generally comparable to the mean score for the HC/APAP alone treatment group, which in turn, was greater than the mean score for the placebo group. The mean PEAKPID scores presented in Table 63B varied among treatment groups, and were greater for the HC/APAP alone or HC/APAP-NTX combination treatment groups compared to the placebo group. Compared to all other groups, the mean PEAKPID scores were highest for the 0.001 mg NTX combination treatment group.

TABLE 63A
Efficacy Results - Means and Standard Deviations for the MAXPAR
(Safety Patients)

| MAXIMUM PAIN RELIEF (MAXPAR) | | | | | |
|---|---|---|---|---|---|
| TREATMENT | N | MEAN | SD | SOURCE | P-Value |
| A) Placebo | 50 | 1.46 | 1.30 | TRT | <0.001 |
| B) HC/APAP | 50 | 2.12 | 1.14 | A-B | 0.007 |
| C) W/NTX 1 mg | 50 | 2.21 | 1.18 | A-C | 0.002 |
| D) W/NTX 0.1 mg | 50 | 2.19 | 1.21 | A-D | 0.003 |
| E) W/NTX 0.01 mg | 50 | 1.90 | 1.27 | A-E | 0.069 |
| F) W/NTX 0.001 mg | 50 | 2.52 | 1.13 | A-F | <0.001 |
| | | | | B-C | 0.706 |
| | | | | B-D | 0.787 |
| | | | | B-E | 0.362 |
| | | | | B-F | 0.098 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNIFICANT).

TABLE 63B
Efficacy Results - Means and Standard Deviation
for the Categorical PEAKPID
(Safety Patients)

| CATEGORICAL PEAK PAIN INTENSITY DIFFERENCE | | | | | |
|---|---|---|---|---|---|
| TREATMENT | N | MEAN | SD | SOURCE | P-Value |
| A) Placebo | 50 | 0.70 | 0.93 | TRT | 0.058 |
| B) HC/APAP | 50 | 0.92 | 0.75 | A-B | 0.170 |
| C) W/NTX 1 mg | 50 | 0.96 | 0.80 | A-C | 0.107 |
| D) W/NTX 0.1 mg | 50 | 0.94 | 0.68 | A-D | 0.135 |
| E) W/NTX 0.01 mg | 50 | 0.82 | 0.83 | A-E | 0.454 |
| F) W/NTX 0.001 mg | 50 | 1.20 | 0.78 | A-F | 0.002 |
| | | | | B-C | 0.810 |
| | | | | B-D | 0.901 |
| | | | | B-E | 0.532 |
| | | | | B-F | 0.081 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNIFICANT).

Table 64 presents the summary and analysis of global evaluations. The placebo treatment group had the highest number of subjects who had “poor” global evaluation scores. The 0.001 mg NTX combination treatment group had the highest number of subjects with a total of “excellent”, “very good” and “good” global evaluation scores. The profiles of the global evaluation scores are based on subjects' evaluations.

TABLE 64
Efficacy Results -Patient Global Assessments
(Safety Patients)

| | | | | | VERY | | P-Value | P-Value | |
|---|---|---|---|---|---|---|---|---|---|
| | | POOR | FAIR | GOOD | GOOD | EXCELLENT | vs. | vs. | |
| TREATMENT | N | (0) | (1) | (2) | (3) | (4) | Placebo | HC/APAP | P-Value |
| A) Placebo | 50 | 26 (52%) | 11 (22%) | 8 (16%) | 5 (10%) | 0 (0%) | | | 0.017 |
| B) HC/APAP | 50 | 13 (26%) | 15 (30%) | 12 (24%) | 6 (12%) | 4 (8%) | 0.045 | | |
| C) W/NTX 1 mg | 50 | 12 (24%) | 12 (24%) | 15 (30%) | 7 (14%) | 4 (8%) | 0.021 | 0.942 | |
| D) W/NTX 0.1 mg | 50 | 15 (30%) | 8 (16%) | 15 (30%) | 9 (18%) | 3 (6%) | 0.048 | 0.506 | |
| E) W/NTX 0.01 mg | 50 | 13 (26%) | 19 (38%) | 8 (16%) | 10 (20%) | 0 (0%) | 0.045 | 0.184 | |
| F) W/NTX 0.001 mg | 50 | 9 (18%) | 11 (22%) | 14 (28%) | 13 (26%) | 3 (6%) | 0.003 | 0.383 | |
| TOTAL | 300 | 88 (29%) | 76 (25%) | 72 (24%) | 50 (17%) | 14 (5%) | | | |

OVERALL P-VALUE (AND ANY PAIRWISE RESULTS) FROM THE COCHRAN-MANTEL-HAENSZEL TEST FOR ROW MEAN SCORES.

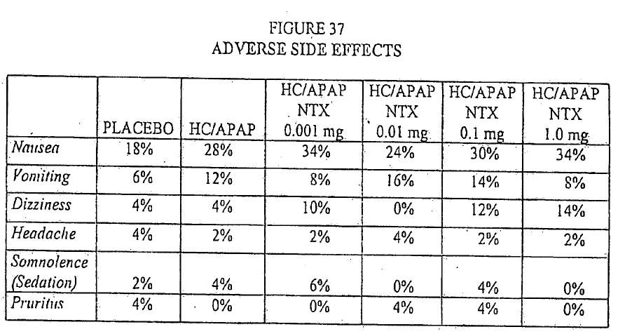

The majority of adverse events reported were categorized as digestive (nausea or vomiting) or nervous system (dizziness or sedation) as further shown in Tables 65A and 65B. FIG. 37 represents a summary of exemplary adverse side effects that may be attenuated according to methods and compositions of the invention.

TABLE 65A
Summary of Adverse Events by Body System and Preferred Term
(Safety Patients)

| | | Total | No. Of | Total | | | |
|---|---|---|---|---|---|---|---|
| Body System | | No. Of | Subjects | No. Of | Severity | | |
| Adverse Events | Treatment | Subjects | W/Event | Events | Mild | Moderate | Severe |
| ALL BODY SYSTEMS | A) PLACEBO | 50 | 14 (28%) | 14 (28%) | 4 (8%) | 8 (16%) | 2 (4%) |
| | B) HC/APAP | 50 | 15 (30%) | 15 (30%) | 3 (6%) | 12 (24%) | 0 (0%) |
| | C) W/NTX 1 mg | 50 | 23 (46%) | 23 (46%) | 5 (10%) | 13 (26%) | 5 (10%) |
| | D) W/NTX 0.1 mg | 50 | 21 (42%) | 21 (42%) | 6 (12%) | 13 (26%) | 2 (4%) |
| | E) W/NTX 0.01 mg | 50 | 21 (42%) | 21 (42%) | 7 (14%) | 12 (24%) | 2 (4%) |
| | F) W/NTX 0.001 mg | 50 | 20 (40%) | 20 (40%) | 3 (6%) | 16 (32%) | 1 (2%) |
| | TOTAL | 300 | 114 (38%) | 114 (38%) | | | |
| EAR AND LABRYRINTH | A) PLACEBO | 50 | 0 (0%) | | | | |
| DISORDERS | B) HC/APAP | 50 | 0 (0%) | | | | |
| | C) W/NTX 1 mg | 50 | 1 (2%) | 1 (2%) | 0 (0%) | 1 (2%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| TINNITUS | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 0 (0%) | 1 (2%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| EYE DISORDERS | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 0 (0%) | 0 (0%) | 1 (2%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| VISION BLURRED | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 0 (0%) | 0 (0%) | 1 (2%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| GASTROINTESTINAL | A) PLACEBO | 50 | 10 (20%) | 10 (20%) | 3 (6%) | 7 (14%) | 0 (0%) |
| DISORDERS | B) HC/APAP | 50 | 14 (28%) | 14 (28%) | 3 (6%) | 11 (22%) | 0 (0%) |
| | C) W/NTX 1 mg | 50 | 17 (34%) | 17 (34%) | 4 (8%) | 10 (20%) | 3 (6%) |
| | D) W/NTX 0.1 mg | 50 | 16 (32%) | 16 (32%) | 3 (6%) | 11 (22%) | 2 (4%) |
| | E) W/NTX 0.01 mg | 50 | 17 (34%) | 17 (34%) | 6 (12%) | 9 (18%) | 2 (4%) |
| | F) W/NTX 0.001 mg | 50 | 18 (36%) | 18 (36%) | 5 (10%) | 12 (24%) | 1 (2%) |
| | TOTAL | 300 | 92 (31%) | | | | |
| ABDOMINAL | A) PLACEBO | 50 | 0 (0%) | | | | |
| DISTENSION | B) HC/APAP | 50 | 1 (2%) | 1 (2%) | 0 (0%) | 1 (2%) | 0 (0%) |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| ABDOMINAL PAIN NOS | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 1 (2%) | 1 (2%) | 0 (0%) | 1 (2%) | 0 (0%) |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| ABDOMINAL PAIN | A) PLACEBO | 50 | 0 (0%) | | | | |
| UPPER | B) HC/APAP | 50 | 0 (0%) | | | | |
| | C) W/NTX 1 mg | 50 | 1 (2%) | 1 (2%) | 0 (0%) | 1 (2%) | 0 (0%) |
| | D) W/NTX 0.1 mg | 50 | 0 (0%) | | | | |
| | E) W/NTX 0.01 mg | 50 | 1 (2%) | 1 (2%) | 0 (0%) | 0 (0%) | 1 (2%) |
| | F) W/NTX 0.001 mg | 50 | 0 (0%) | | | | |
| | TOTAL | 300 | 2 (1%) | | | | |
| CONSTIPATION | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 1 (2%) | 1 (2%) | 0 (0%) | 1 (2%) | 0 (0%) |
| | E) W/NTX 0.01 mg | 50 | 0 (0%) | | | | |
| | F) W/NTX 0.001 mg | 50 | 1 (2%) | 1 (2%) | 1 (2%) | 0 (0%) | 0 (0%) |
| | TOTAL | 300 | 2 (1%) | | | | |
| DIARRHEA NOS | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 1 (2%) | 0 (0%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 1 (2%) | 1 (2%) | 0 (0%) | 0 (0%) | 1 (2%) |
| | TOTAL | 300 | 2 (1%) | | | | |
| DYSPEPSIA | A) PLACEBO | 50 | 1 (2%) | 1 (2%) | 0 (0%) | 1 (2%) | 0 (0%) |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| FLATULENCE | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 0 (0%) | 1 (2%) | 0 (0%) |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 0 (0%) | | | | |
| | TOTAL | 300 | 2 (1%) | | | | |
| NAUSEA | A) PLACEBO | 50 | 9 (18%) | 9 (18%) | 3 (6%) | 6 (12%) | 0 (0%) |
| | B) HC/APAP | 50 | 14 (28%) | 14 (28%) | 3 (6%) | 11 (22%) | 0 (0%) |
| | C) W/NTX 1 mg | 50 | 17 (34%) | 17 (34%) | 5 (10%) | 9 (18%) | 3 (6%) |
| | D) W/NTX 0.1 mg | 50 | 15 (30%) | 15 (30%) | 6 (12%) | 9 (18%) | 0 (0%) |
| | E) W/NTX 0.01 mg | 50 | 12 (24%) | 12 (24%) | 5 (10%) | 6 (12%) | 1 (2%) |
| | F) W/NTX 0.001 mg | 50 | 17 (34%) | 17 (34%) | 4 (8%) | 13 (26%) | 0 (0%) |
| | TOTAL | 300 | 84 (28%) | | | | |
| SORE THROAT NOS | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 0 (0%) | | | | |
| | TOTAL | 300 | 1 (<1%) | | | | |
| VOMITING NOS | A) PLACEBO | 50 | 3 (6%) | 3 (6%) | 1 (2%) | 2 (4%) | 0 (0%) |
| | B) HC/APAP | 50 | 6 (12%) | 6 (12%) | 1 (2%) | 5 (10%) | 0 (0%) |
| | C) W/NTX 1 mg | 50 | 4 (8%) | 4 (8%) | 0 (0%) | 4 (8%) | 0 (0%) |
| | D) W/NTX 0.1 mg | 50 | 7 (14%) | 7 (14%) | 2 (4%) | 3 (6%) | 2 (4%) |
| | E) W/NTX 0.01 mg | 50 | 8 (16%) | 8 (16%) | 2 (4%) | 5 (10%) | 1 (2%) |
| | F) W/NTX 0.001 mg | 50 | 4 (8%) | 4 (8%) | 0 (0%) | 4 (8%) | 0 (0%) |
| | TOTAL | 300 | 32 (11%) | | | | |
| GENERAL DISORDERS | A) PLACEBO | 50 | 0 (0%) | | | | |
| AND ADMIN. SITE | B) HC/APAP | 50 | 1 (2%) | 1 (2%) | 1 (2%) | 0 (0%) | 0 (0%) |
| CONDITIONS | C) W/NTX 1 mg | 50 | 1 (2%) | 1 (2%) | 0 (0%) | 0 (0%) | 1 (2%) |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 1 (2%) | 1 (2%) | 0 (0%) | 1 (2%) | 0 (0%) |
| | TOTAL | 300 | 4 (1%) | | | | |
| APPLICATION SITE | A) PLACEBO | 50 | 0 (0%) | | | | |
| BLEEDING | B) HC/APAP | 50 | 0 (0%) | | | | |
| | C) W/NTX 1 mg | 50 | 1 (2%) | 1 (2%) | 0 (0%) | 0 (0%) | 1 (2%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| FATIGUE | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 1 (2%) | 1 (2%) | 0 (0%) | 1 (2%) | 0 (0%) |
| | TOTAL | 300 | 1 (<1%) | | | | |
| PYREXIA | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 0 (0%) | | | | |
| | TOTAL | 300 | 1 (<1%) | | | | |
| RIGORS | A) PLACEBO | 50 | 0 (0%) | | | | |
| | B) HC/APAP | 50 | 1 (2%) | 1 (2%) | 1 (2%) | 0 (0%) | 0 (0%) |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| INJURY AND POISONING | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 1 (2%) | 0 (0%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| ABRASION NOS | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 1 (2%) | 0 (0%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| INVESTIGATIONS | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 1 (2%) | 0 (0%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| BLOOD PRESSURE | A) PLACEBO | 50 | 0 (0%) | | | | |
| INCREASED | B) HC/APAP | 50 | 0 (0%) | | | | |
| | C) W/NTX 1 mg | 50 | 1 (2%) | 1 (2%) | 1 (2%) | 0 (0%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| MUSCULOSKELETAL, | A) PLACEBO | 50 | 0 (0%) | | | | |
| CONNECT. TISSUE AND | B) HC/APAP | 50 | 0 (0%) | | | | |
| BONE DISORDERS | C) W/NTX 1 mg | 50 | 0 (0%) | | | | |
| | D) W/NTX 0.1 mg | 50 | 1 (2%) | 1 (2%) | 1 (2%) | 0 (0%) | 0 (0%) |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| NECK PAIN | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 1 (2%) | 1 (2%) | 1 (2%) | 0 (0%) | 0 (0%) |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| NERVOUS SYSTEM | A) PLACEBO | 50 | 6 (12%) | 6 (12%) | 2 (4%) | 2 (4%) | 2 (4%) |
| DISORDERS | B) HC/APAP | 50 | 6 (12%) | 6 (12%) | 2 (4%) | 4 (8%) | 0 (0%) |
| | C) W/NTX 1 mg | 50 | 8 (16%) | 8 (16%) | 2 (4%) | 5 (10%) | 1 (2%) |
| | D) W/NTX 0.1 mg | 50 | 11 (22%) | 11 (22%) | 6 (12%) | 5 (10%) | 0 (0%) |
| | E) W/NTX 0.01 mg | 50 | 4 (8%) | 4 (8%) | 1 (2%) | 2 (4%) | 1 (2%) |
| | F) W/NTX 0.001 mg | 50 | 10 (20%) | 10 (20%) | 2 (4%) | 8 (16%) | 0 (0%) |
| | TOTAL | 300 | 45 (15%) | | | | |
| DIZZINESS EXC. | A) PLACEBO | 50 | 2 (4%) | 2 (4%) | 1 (2%) | 1 (2%) | 0 (0%) |
| VERTIGO | B) HC/APAP | 50 | 2 (4%) | 2 (4%) | 1 (2%) | 1 (2%) | 0 (0%) |
| | C) W/NTX 1 mg | 50 | 7 (14%) | 7 (14%) | 3 (6%) | 3 (6%) | 1 (2%) |
| | D) W/NTX 0.1 mg | 50 | 6 (12%) | 6 (12%) | 4 (8%) | 2 (4%) | 0 (0%) |
| | E) W/NTX 0.01 mg | 50 | 0 (0%) | | | | |
| | F) W/NTX 0.001 mg | 50 | 5 (10%) | 5 (10%) | 2 (4%) | 3 (6%) | 0 (0%) |
| | TOTAL | 300 | 22 (7%) | | | | |
| HEADACHE NOS | A) PLACEBO | 50 | 2 (4%) | 2 (4%) | 0 (0%) | 1 (2%) | 1 (2%) |
| | B) HC/APAP | 50 | 1 (2%) | 1 (2%) | 1 (2%) | 0 (0%) | 0 (0%) |
| | C) W/NTX 1 mg | 50 | 1 (2%) | 1 (2%) | 0 (0%) | 1 (2%) | 0 (0%) |
| | D) W/NTX 0.1 mg | 50 | 1 (2%) | 1 (2%) | 1 (2%) | 0 (0%) | 0 (0%) |
| | E) W/NTX 0.01 mg | 50 | 2 (4%) | 2 (4%) | 1 (2%) | 1 (2%) | 0 (0%) |
| | F) W/NTX 0.001 mg | 50 | 1 (2%) | 1 (2%) | 0 (0%) | 1 (2%) | 0 (0%) |
| | TOTAL | 300 | 8 (3%) | | | | |
| MIGRAINE NOS | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 1 (2%) | 1 (2%) | 0 (0%) | 0 (0%) | 1 (2%) |
| | F) W/NTX 0.001 mg | 50 | 0 (0%) | | | | |
| | TOTAL | 300 | 1 (<1%) | | | | |
| SEDATION | A) PLACEBO | 50 | 1 (2%) | 1 (2%) | 1 (2%) | 0 (0%) | 0 (0%) |
| | B) HC/APAP | 50 | 2 (4%) | 2 (4%) | 1 (2%) | 1 (2%) | 0 (0%) |
| | C) W/NTX 1 mg | 50 | 0 (0%) | | | | |
| | D) W/NTX 0.1 mg | 50 | 2 (4%) | 2 (4%) | 0 (0%) | 2 (4%) | 0 (0%) |
| | E) W/NTX 0.01 mg | 50 | 0 (0%) | | | | |
| | F) W/NTX 0.001 mg | 50 | 3 (6%) | 3 (6%) | 0 (0%) | 3 (6%) | 0 (0%) |
| | TOTAL | 300 | 8 (3%) | | | | |
| SYNCOPE | A) PLACEBO | 50 | 1 (2%) | 1 (2%) | 0 (0%) | 0 (0%) | 1 (2%) |
| | B) HC/APAP | 50 | 1 (2%) | 1 (2%) | 0 (0%) | 1 (2%) | 0 (0%) |
| | C) W/NTX 1 mg | 50 | 2 (4%) | 2 (4%) | 0 (0%) | 2 (4%) | 0 (0%) |
| | D) W/NTX 0.1 mg | 50 | 2 (4%) | 2 (4%) | 1 (2%) | 1 (2%) | 0 (0%) |
| | E) W/NTX 0.01 mg | 50 | 1 (2%) | 1 (2%) | 0 (0%) | 1 (2%) | 0 (0%) |
| | F) W/NTX 0.001 mg | 50 | 1 (2%) | 1 (2%) | 0 (0%) | 1 (2%) | 0 (0%) |
| | TOTAL | 300 | 8 (3%) | | | | |
| TREMOR NEC | A) PLACEBO | 50 | 0 (0%) | | | | |
| | B) HC/APAP | 50 | 1 (2%) | 1 (2%) | 0 (0%) | 1 (2%) | 0 (0%) |
| | C) W/NTX 1 mg | 50 | 0 (0%) | | | | |
| | D) W/NTX 0.1 mg | 50 | 1 (2%) | 1 (2%) | 1 (2%) | 0 (0%) | 0 (0%) |
| | E) W/NTX 0.01 mg | 50 | 0 (0%) | | | | |
| | F) W/NTX 0.001 mg | 50 | 0 (0%) | | | | |
| | TOTAL | 300 | 2 (1%) | | | | |
| PHYCHIATRIC | A) PLACEBO | 50 | 0 (0%) | | | | |
| DISORDERS | B) HC/APAP | 50 | 1 (2%) | 1 (2%) | 0 (0%) | 1 (2%) | 0 (0%) |
| | C) W/NTX 1 mg | 50 | 2 (4%) | 2 (4%) | 0 (0%) | 2 (4%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 3 (1%) | | | | |
| ANXIETY NEC | A) PLACEBO | 50 | 0 (0%) | | | | |
| | B) HC/APAP | 50 | 1 (2%) | 1 (2%) | 0 (0%) | 1 (2%) | 0 (0%) |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| CRYING | A) PLACEBO | 50 | 0 (0%) | | | | |
| | B) HC/APAP | 50 | 1 (2%) | 1 (2%) | 0 (0%) | 1 (2%) | 0 (0%) |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| NERVOUSNESS | A) PLACEBO | 50 | 0 (0%) | | | | |
| | B) HC/APAP | 50 | 0 (0%) | | | | |
| | C) W/NTX 1 mg | 50 | 2 (4%) | 2 (4%) | 0 (0%) | 2 (4%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 2 (1%) | | | | |
| RENAL AND URINARY | A) PLACEBO | 50 | 0 (0%) | | | | |
| DISORDERS | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 1 (2%) | 1 (2%) | 1 (2%) | 0 (0%) | 0 (0%) |
| | F) W/NTX 0.001 mg | 50 | 0 (0%) | | | | |
| | TOTAL | 300 | 1 (<1%) | | | | |
| DIFFICULTY IN | A) PLACEBO | 50 | 0 (0%) | | | | |
| MICTURITION | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 1 (2%) | 1 (2%) | 1 (2%) | 0 (0%) | 0 (0%) |
| | F) W/NTX 0.001 mg | 50 | 0 (0%) | | | | |
| | TOTAL | 300 | 1 (<1%) | | | | |
| RESPIRATORY, | A) PLACEBO | 50 | 0 (0%) | | | | |
| THORACIC | B) HC/APAP | 50 | 1 (2%) | 1 (2%) | 0 (0%) | 1 (2%) | 0 (0%) |
| AND MEDIASTINAL | C) W/NTX 1 mg | 50 | 0 (0%) | | | | |
| DISORDERS | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| RESPIRATORY | A) PLACEBO | 50 | 0 (0%) | | | | |
| DISORDER NOS | B) HC/APAP | 50 | 1 (2%) | 1 (2%) | 0 (0%) | 1 (2%) | 0 (0%) |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| SKIN AND | A) PLACEBO | 50 | 2 (4%) | 2 (4%) | 1 (2%) | 1 (2%) | 0 (0%) |
| SUBCUTANEOUS TISSUE | B) HC/APAP | 50 | 1 (2%) | 1 (2%) | 0 (0%) | 1 (2%) | 0 (0%) |
| DISORDERS | C) W/NTX 1 mg | 50 | 4 (8%) | 4 (8%) | 0 (0%) | 4 (8%) | 0 (0%) |
| | D) W/NTX 0.1 mg | 50 | 4 (8%) | 4 (8%) | 1 (2%) | 3 (6%) | 0 (0%) |
| | E) W/NTX 0.01 mg | 50 | 4 (8%) | 4 (8%) | 1 (2%) | 3 (6%) | 0 (0%) |
| | F) W/NTX 0.001 mg | 50 | 2 (4%) | 2 (4%) | 0 (0%) | 2 (4%) | 0 (0%) |
| | TOTAL | 300 | 17 (6%) | | | | |
| FACE OEDMA | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 0 (0%) | | | | |
| | TOTAL | 300 | 1 (<1%) | | | | |
| PRURITUS NOS | A) PLACEBO | 50 | 2 (4%) | 2 (4%) | 1 (2%) | 1 (2%) | 0 (0%) |
| | B) HC/APAP | 50 | 0 (0%) | | | | |
| | C) W/NTX 1 mg | 50 | 0 (0%) | | | | |
| | D) W/NTX 0.1 mg | 50 | 2 (4%) | 2 (4%) | 1 (2%) | 1 (2%) | 0 (0%) |
| | E) W/NTX 0.01 mg | 50 | 2 (4%) | 2 (4%) | 1 (2%) | 1 (2%) | 0 (0%) |
| | F) W/NTX 0.001 mg | 50 | 0 (0%) | | | | |
| | TOTAL | 300 | 6 (2%) | | | | |
| SWEATING | A) PLACEBO | 50 | 0 (0%) | | | | |
| INCREASED | B) HC/APAP | 50 | 1 (2%) | 1 (2%) | 0 (0%) | 1 (2%) | 0 (0%) |
| | C) W/NTX 1 mg | 50 | 4 (8%) | 4 (8%) | 0 (0%) | 4 (8%) | 0 (0%) |
| | D) W/NTX 0.1 mg | 50 | 1 (2%) | 1 (2%) | 0 (0%) | 1 (2%) | 0 (0%) |
| | E) W/NTX 0.01 mg | 50 | 1 (2%) | 1 (2%) | 0 (0%) | 1 (2%) | 0 (0%) |
| | F) W/NTX 0.001 mg | 50 | 2 (4%) | 2 (4%) | 0 (0%) | 2 (4%) | 0 (0%) |
| | TOTAL | 300 | 9 (3%) | | | | |
| URTICARIA NOS | A) PLACEBO | 50 | 0 (0%) | | | | |
| | B) HC/APAP | 50 | 0 (0%) | | | | |
| | C) W/NTX 1 mg | 50 | 0 (0%) | | | | |
| | D) W/NTX 0.1 mg | 50 | 1 (2%) | 1 (2%) | 0 (0%) | 1 (2%) | 0 (0%) |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| VASCULAR | A) PLACEBO | 50 | 1 (2%) | 1 (2%) | 1 (2%) | 0 (0%) | 0 (0%) |
| DISORDERS | B) HC/APAP | 50 | 0 (0%) | | | | |
| | C) W/NTX 1 mg | 50 | 4 (8%) | 4 (8%) | 0 (0%) | 3 (6%) | 1 (2%) |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 1 (2%) | 1 (2%) | 0 (0%) | 1 (2%) | 0 (0%) |
| | TOTAL | 300 | 7 (2%) | | | | |
| FLUSHING | A) PLACEBO | 50 | 1 (2%) | 1 (2%) | 1 (2%) | 0 (0%) | 0 (0%) |
| | B) HC/APAP | 50 | 0 (0%) | | | | |
| | C) W/NTX 1 mg | 50 | 0 (0%) | | | | |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 1 (<1%) | | | | |
| HOT FLUSHES NOS | A) PLACEBO | 50 | 0 (0%) | | | | |
| | B) HC/APAP | 50 | 0 (0%) | | | | |
| | C) W/NTX 1 mg | 50 | 2 (4%) | 2 (4%) | 0 (0%) | 1 (2%) | 1 (2%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 0 (0%) | | | | |
| | TOTAL | 300 | 2 (1%) | | | | |
| HYPERTENSION NOS | A) PLACEBO | 50 | 0 (0%) | | | | |
| | B) HC/APAP | 50 | 0 (0%) | | | | |
| | C) W/NTX 1 mg | 50 | 1 (2%) | 1 (2%) | 0 (0%) | 1 (2%) | 0 (0%) |
| | D) W/NTX 0.1 mg | 50 | 0 (0%) | | | | |
| | E) W/NTX 0.01 mg | 50 | 1 (2%) | 1 (2%) | 0 (0%) | 1 (2%) | 0 (0%) |
| | F) W/NTX 0.001 mg | 50 | 0 (0%) | | | | |
| | TOTAL | 300 | 2 (1%) | | | | |
| PALLOR | A) PLACEBO | 50 | 0 (0%) | | | | |
| | B) HC/APAP | 50 | 0 (0%) | | | | |
| | C) W/NTX 1 mg | 50 | 2 (4%) | 2 (4%) | 0 (0%) | 2 (4%) | 0 (0%) |
| | D) W/NTX 0.1 mg | 50 | 0 (0%) | | | | |
| | E) W/NTX 0.01 mg | 50 | 0 (0%) | | | | |
| | F) W/NTX 0.001 mg | 50 | 1 (2%) | 1 (2%) | 0 (0%) | 1 (2%) | 0 (0%) |
| | TOTAL | 300 | 3 (1%) | | | | |

NOTE:
AT EACH LEVEL OF SUMMATION (BODY SYSTEM AND PREFERRED TERMS), PATIENTS REPORTING MORE THAN ONE EVENT ARE COUNTED ONLY ONCE. PERCENTAGES OF PATIENTS FOR EACH TREATMENT GROUP ARE ALSO GIVEN.

TABLE 65B
Summary of Adverse Events by Body System and Preferred Term
(Safety Patients)

| | | Total | No. Of | Total | | | |
|---|---|---|---|---|---|---|---|
| Body System | | No. Of | Subjects | No. Of | Severity | | |
| Adverse Events | Treatment | Subjects | W/Event | Events | Mild | Moderate | Severe |
| NAUSEA | A) PLACEBO | 50 | 9 (18%) | 9 (18%) | 3 (6%) | 6 (12%) | 0 (0%) |
| | B) HC/APAP | 50 | 14 (28%) | 14 (28%) | 3 (6%) | 11 (22%) | 0 (0%) |
| | C) W/NTX 1 mg | 50 | 17 (34%) | 17 (34%) | 5 (10%) | 9 (18%) | 3 (6%) |
| | D) W/NTX 0.1 mg | 50 | 15 (30%) | 15 (30%) | 6 (12%) | 9 (18%) | 0 (0%) |
| | E) W/NTX 0.01 mg | 50 | 12 (24%) | 12 (24%) | 5 (10%) | 6 (12%) | 1 (2%) |
| | F) W/NTX 0.001 mg | 50 | 17 (34%) | 17 (34%) | 4 (8%) | 13 (26%) | 0 (0%) |
| | TOTAL | 300 | 84 (28%) | | | | |
| VOMITING NOS | A) PLACEBO | 50 | 3 (6%) | 3 (6%) | 1 (2%) | 2 (4%) | 0 (0%) |
| | B) HC/APAP | 50 | 6 (12%) | 6 (12%) | 1 (2%) | 5 (10%) | 0 (0%) |
| | C) W/NTX 1 mg | 50 | 4 (8%) | 4 (8%) | 0 (0%) | 4 (8%) | 0 (0%) |
| | D) W/NTX 0.1 mg | 50 | 7 (14%) | 7 (14%) | 2 (4%) | 3 (6%) | 2 (4%) |
| | E) W/NTX 0.01 mg | 50 | 8 (16%) | 8 (16%) | 2 (4%) | 5 (10%) | 1 (2%) |
| | F) W/NTX 0.001 mg | 50 | 4 (8%) | 4 (8%) | 0 (0%) | 4 (8%) | 0 (0%) |
| | TOTAL | 300 | 32 (11%) | | | | |
| DIZZINESS EXC. | A) PLACEBO | 50 | 2 (4%) | 2 (4%) | 1 (2%) | 1 (2%) | 0 (0%) |
| VERTIGO | B) HC/APAP | 50 | 2 (4%) | 2 (4%) | 1 (2%) | 1 (2%) | 0 (0%) |
| | C) W/NTX 1 mg | 50 | 7 (14%) | 7 (14%) | 3 (6%) | 3 (6%) | 1 (2%) |
| | D) W/NTX 0.1 mg | 50 | 6 (12%) | 6 (12%) | 4 (8%) | 2 (4%) | 0 (0%) |
| | E) W/NTX 0.01 mg | 50 | 0 (0%) | | | | |
| | F) W/NTX 0.001 mg | 50 | 5 (10%) | 5 (10%) | 2 (4%) | 3 (6%) | 0 (0%) |
| | TOTAL | 300 | 22 (7%) | | | | |
| SEDATION | A) PLACEBO | 50 | 1 (2%) | 1 (2%) | 1 (2%) | 0 (0%) | 0 (0%) |
| | B) HC/APAP | 50 | 2 (4%) | 2 (4%) | 1 (2%) | 1 (2%) | 0 (0%) |
| | C) W/NTX 1 mg | 50 | 0 (0%) | | | | |
| | D) W/NTX 0.1 mg | 50 | 2 (4%) | 2 (4%) | 0 (0%) | 2 (4%) | 0 (0%) |
| | E) W/NTX 0.01 mg | 50 | 0 (0%) | | | | |
| | F) W/NTX 0.001 mg | 50 | 3 (6%) | 3 (6%) | 0 (0%) | 3 (6%) | 0 (0%) |
| | TOTAL | 300 | 8 (3%) | | | | |

NOTE:
AT EACH LEVEL OF SUMMATION (BODY SYSTEM AND PREFERRED TERMS), PATIENTS REPORTING MORE THAN ONE EVENT ARE COUNTED ONLY ONCE. PERCENTAGES OF PATIENTS FOR EACH TREATMENT GROUP ARE ALSO GIVEN.

### EXAMPLE 6

An additional clinical study, this one using hydrocodone with acetaminophen (instead of morphine) alone and in combination with naltrexone, was designed substantially the same as that described in Example 2, with the following differences: (1) six treatment groups with four different doses of NTX (1.0 mg, 0.1 mg, 0.01 mg and 0.001 mg) in combination with hydrocodone 5 mg/acetaminophen 500 mg versus hydrocodone 5 mg/acetaminophen 500 mg (HC/APAP) alone, and versus placebo alone in subjects with moderate to severe pain in a postsurgical dental pain clinical study; (2) the primary efficacy variable was the categorical sum of pain intensity difference scores through 4 hours (SPID-4); and (3) the secondary efficacy variables were: 4, 6 and 8 hour total pain relief scores (TOTPAR-4, TOTPAR-6 and TOTPAR-8); categorical 6 and 8 hour sum of pain intensity difference scores (SPID-6 and SPID-8); categorical pain intensity difference (PID) scores through 8 hours; pain relief (PR) scores through 8 hours; peak categorical PID scores through 8 hours (PEAKPID); peak pain relief score through 8 hours (TOTPAR); time to onset of analgesia (i.e., at least a one category improvement in the pain intensity score); time to onset of meaningful pain relief; time to taking backup medication; percent of patients taking backup medication; and patient overall evaluation of study drug.

The results for females and males separately are shown in the following tables and figures.

A total of 300 subjects were randomized; all 300 subjects were deemed evaluable as shown in Table 66. Table 67 shows the number of female and male subjects separately for each treatment group.

TABLE 66
Patient Enrollment and Evaluability

| | TREATMENTS | | | | | | |
|---|---|---|---|---|---|---|---|
| | | | | | W/NTX | W/NTX | |
| | Placebo | HC/APAP | W/NTX 1 | W/NTX 0.1 | 0.01 | 0.001 | TOTAL |
| Number of Patients | 50 | 50 | 50 | 50 | 50 | 50 | 300 |
| Patients Included in the | 50 (100%) | 50 (100%) | 50 (100%) | 50 (100%) | 50 (100%) | 50 (100%) | 300 (100%) |
| Safety Analyses | | | | | | | |
| Patients Excluded in the | 0 (0%) | 0 (0%) | 0 (0%) | 0 (0%) | 0 (0%) | 0 (0%) | 0 (0%) |
| Safety Analyses | | | | | | | |
| Patients Included in the | 50 (100%) | 50 (100%) | 50 (100%) | 50 (100%) | 50 (100%) | 50 (100%) | 300 (100%) |
| Efficacy Analyses | | | | | | | |
| Patients Excluded in the | 0 (0%) | 0 (0%) | 0 (0%) | 0 (0%) | 0 (0%) | 0 (0%) | 0 (0%) |
| Efficacy Analyses | | | | | | | |

TABLE 67
Patient Characteristics (Safety Patients)

| | | | | | | W/NTX | | |
|---|---|---|---|---|---|---|---|---|
| Sex | Placebo | NC/APAP | W/NTX 1 | W/NTX 0.1 | W/NTX 0.01 | 0.001 | TOTAL | P-Value |
| Female | 28 (56%) | 34 (68%) | 31 (62%) | 35 (70%) | 31 (62%) | 30 (60%) | 189 (63%) | 0.716^{b} |
| Male | 22 (44%) | 16 (32%) | 19 (38%) | 15 (30%) | 19 (38%) | 20 (40%) | 111 (37%) | |

^{b}P-VALUE FROM A LIKELIHOOD RATIO CHI-SQUARE TEST. FOR RACE, NON-CAUCASIANS WERE COMBINED AS ONE CATEGORY FOR THE ANALYSIS.

The total pain relief scores (TOTPAR) results for 4, 6 and 8 hours are summarized in Tables 68A for females and 68B for males.

In females, all of the active treatment groups exhibited mean TOTPAR scores that were higher than the placebo group score. The HC/APAP alone treatment group had mean TOTPAR scores that were higher than the scores for the four NTX combination groups.

In males, all of the active treatment groups exhibited mean TOTPAR scores that were higher than the placebo group score. Both the 0.1 mg NTX and 0.001 mg NTX combination treatment groups had higher mean TOTPAR scores than the HC/APAP alone group. The 0.001 mg NTX combination group had the highest mean TOTPAR scores for the 4, 6 and 8 hours.

TABLE 68A
Efficacy Results - Means and Standard Deviations for TOTPARs
(Trapezoidal Method)
Female Safety Patients

| TOTAL PAIN RELIEF SCORES | | | | | |
|---|---|---|---|---|---|
| TREATMENT | N | MEAN | SD | SOURCE | P-VALUE |

TOTAL PAIN RELIEF SCORES (4 HOURS)

| | | | | | |
|---|---|---|---|---|---|
| A) Placebo | 28 | 1.56 | 2.23 | TRT | 0.012 |
| B) HC/APAP | 34 | 4.55 | 4.15 | B-A | 0.001 |
| C) With NTX 1 | 30 | 4.42 | 3.88 | C-A | 0.002 |
| D) W/NTX 0.1 | 35 | 4.35 | 3.26 | D-A | 0.002 |
| E) W/NTX 0.01 | 31 | 3.76 | 4.07 | E-A | 0.018 |
| F) W/NTX 0.001 | 30 | 4.28 | 3.00 | F-A | 0.004 |
| | | | | C-B | 0.882 |
| | | | | D-B | 0.810 |
| | | | | E-B | 0.367 |
| | | | | F-B | 0.760 |

TOTAL PAIN RELIEF SCORES (6 HOURS)

| | | | | | |
|---|---|---|---|---|---|
| A) Placebo | 28 | 1.65 | 2.59 | TRT | 0.034 |
| B) HC/APAP | 34 | 5.56 | 6.04 | B-A | 0.001 |
| C) With NTX 1 | 30 | 4.96 | 5.01 | C-A | 0.008 |
| D) W/NTX 0.1 | 35 | 4.69 | 3.98 | D-A | 0.012 |
| E) W/NTX 0.01 | 31 | 4.53 | 5.57 | E-A | 0.020 |
| F) W/NTX 0.001 | 30 | 4.71 | 3.97 | F-A | 0.014 |
| | | | | C-B | 0.612 |
| | | | | D-B | 0.441 |
| | | | | E-B | 0.379 |
| | | | | F-B | 0.471 |

TOTAL PAIN RELIEF SCORES (8 HOURS)

| | | | | | |
|---|---|---|---|---|---|
| A) Placebo | 28 | 1.65 | 2.59 | TRT | 0.036 |
| B) HC/APAP | 34 | 5.81 | 6.56 | B-A | 0.001 |
| C) With NTX 1 | 30 | 5.23 | 5.87 | C-A | 0.008 |
| D) W/NTX 0.1 | 35 | 4.69 | 3.98 | D-A | 0.019 |
| E) W/NTX 0.01 | 30 | 4.20 | 5.37 | E-A | 0.056 |
| F) W/NTX 0.001 | 30 | 4.96 | 4.77 | F-A | 0.014 |
| | | | | C-B | 0.647 |
| | | | | D-B | 0.357 |
| | | | | E-B | 0.206 |
| | | | | F-B | 0.503 |

MEANS GIVEN ARE LEAST SQUARE MEANS.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE 68B
Efficacy Results - Means and Standard Deviations for TOTPARs
(Trapezoidal Method)
Male Safety Patients

| TOTAL PAIN RELIEF SCORES | | | | | |
|---|---|---|---|---|---|
| TREATMENT | N | MEAN | SD | SOURCE | P-VALUE |

TOTAL PAIN RELIEF SCORES (4 HOURS)

| | | | | | |
|---|---|---|---|---|---|
| A) Placebo | 22 | 2.16 | 2.90 | TRT | 0.007 |
| B) HC/APAP | 16 | 3.73 | 3.66 | B-A | 0.212 |
| C) With NTX 1 | 19 | 3.45 | 3.75 | C-A | 0.284 |
| D) W/NTX 0.1 | 15 | 4.17 | 4.05 | D-A | 0.117 |
| E) W/NTX 0.01 | 19 | 2.99 | 2.83 | E-A | 0.490 |
| F) W/NTX 0.001 | 20 | 6.70 | 5.19 | F-A | <0.001 |
| | | | | C-B | 0.824 |
| | | | | D-B | 0.748 |
| | | | | E-B | 0.565 |
| | | | | F-B | 0.022 |

TOTAL PAIN RELIEF SCORES (6 HOURS)

| | | | | | |
|---|---|---|---|---|---|
| A) Placebo | 22 | 2.48 | 4.08 | TRT | 0.008 |
| B) HC/APAP | 16 | 4.45 | 5.01 | B-A | 0.251 |
| C) With NTX 1 | 19 | 3.79 | 4.46 | C-A | 0.423 |
| D) W/NTX 0.1 | 15 | 4.97 | 5.61 | D-A | 0.155 |
| E) W/NTX 0.01 | 19 | 3.02 | 2.89 | E-A | 0.743 |
| F) W/NTX 0.001 | 20 | 8.40 | 7.79 | F-A | <0.001 |
| | | | | C-B | 0.707 |
| | | | | D-B | 0.780 |
| | | | | E-B | 0.417 |
| | | | | F-B | 0.025 |

TOTAL PAIN RELIEF SCORES (8 HOURS)

| | | | | | |
|---|---|---|---|---|---|
| A) Placebo | 22 | 2.82 | 5.52 | TRT | 0.014 |
| B) HC/APAP | 16 | 4.77 | 5.64 | B-A | 0.357 |
| C) With NTX 1 | 19 | 3.82 | 4.53 | C-A | 0.621 |
| D) W/NTX 0.1 | 15 | 5.77 | 7.45 | D-A | 0.171 |
| E) W/NTX 0.01 | 19 | 3.02 | 2.89 | E-A | 0.924 |
| F) W/NTX 0.001 | 20 | 9.48 | 9.94 | F-A | 0.001 |
| | | | | C-B | 0.662 |
| | | | | D-B | 0.661 |
| | | | | E-B | 0.422 |
| | | | | F-B | 0.030 |

MEANS GIVEN ARE LEAST SQUARE MEANS.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

The sum of pain intensity difference scores (SPID) results at 4, 6 and 8 hours are summarized in Tables 69A for females and 69B for males and the 4 hour SPID results are shown in FIGS. 38B for females and 38C for males. In females, all of the active treatment groups exhibited mean SPID scores that were higher than the placebo group score. The HC/APAP along group had the highest mean SPID scores throughout the 4, 6 and 8 hours. In males, all of the active treatment groups exhibited mean SPID scores that were higher than the placebo group score. Both the 0.1 ma NTX and the 0.001 mg NTX combination groups had higher mean SPID scores than the HC/APAP alone group. The 0.001 mg NTX combination group had the highest mean SPID scores for the 4, 6 and 8 hours.

TABLE 69A
Efficacy Results - Means and Standard Deviations for the SPIDS
(Trapezoidal Method)
Female Safety Patients

| CATEGORICAL SPID SCORES | | | | | |
|---|---|---|---|---|---|
| TREATMENT | N | MEAN | SD | SOURCE | P-VALUE |

CATEGORICAL SPID SCORES (4 HOURS)

| | | | | | |
|---|---|---|---|---|---|
| A) Placebo | 28 | −0.41 | 2.21 | TRT | 0.027 |
| B) HC/APAP | 34 | 1.66 | 2.69 | B-A | 0.001 |
| C) With NTX 1 | 30 | 1.34 | 2.74 | C-A | 0.008 |
| D) W/NTX 0.1 | 35 | 1.43 | 1.75 | D-A | 0.004 |
| E) W/NTX 0.01 | 31 | 1.27 | 2.79 | E-A | 0.011 |
| F) W/NTX 0.001 | 30 | 1.22 | 2.69 | F-A | 0.014 |
| | | | | C-B | 0.617 |
| | | | | D-B | 0.708 |
| | | | | E-B | 0.537 |
| | | | | F-B | 0.486 |

CATEGORICAL SPID SCORES (6 HOURS)

| | | | | | |
|---|---|---|---|---|---|
| A) Placebo | 28 | −1.03 | 3.11 | TRT | 0.028 |
| B) HC/APAP | 34 | 1.97 | 3.85 | B-A | <0.001 |
| C) With NTX 1 | 30 | 1.05 | 3.74 | C-A | 0.024 |
| D) W/NTX 0.1 | 35 | 1.40 | 2.28 | D-A | 0.007 |
| E) W/NTX 0.01 | 31 | 1.40 | 4.05 | E-A | 0.008 |
| F) W/NTX 0.001 | 30 | 1.00 | 3.72 | F-A | 0.028 |
| | | | | C-B | 0.299 |
| | | | | D-B | 0.501 |
| | | | | E-B | 0.517 |
| | | | | F-B | 0.273 |

CATEGORICAL SPID SCORES (8 HOURS)

| | | | | | |
|---|---|---|---|---|---|
| A) Placebo | 28 | −1.67 | 4.01 | TRT | 0.031 |
| B) HC/APAP | 34 | 1.86 | 4.35 | B-A | <0.001 |
| C) With NTX 1 | 30 | 0.62 | 4.64 | C-A | 0.035 |
| D) W/NTX 0.1 | 35 | 1.21 | 2.58 | D-A | 0.006 |
| E) W/NTX 0.01 | 30 | 0.74 | 4.06 | E-A | 0.027 |
| F) W/NTX 0.001 | 30 | 0.75 | 4.80 | F-A | 0.026 |
| | | | | C-B | 0.229 |
| | | | | D-B | 0.508 |
| | | | | E-B | 0.275 |
| | | | | F-B | 0.282 |

MEANS GIVEN ARE LEAST SQUARE MEANS.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE 69B
Efficacy Results - Means and Standard Deviations for the SPIDS
Trapezoidal Method)
Male Safety Patients

| CATEGORICAL SPID SCORES | | | | | |
|---|---|---|---|---|---|
| TREATMENT | N | MEAN | SD | SOURCE | P-VALUE |

CATEGORICAL SPID SCORES (4 HOURS)

| | | | | | |
|---|---|---|---|---|---|
| A) Placebo | 22 | 0.03 | 2.89 | TRT | 0.018 |
| B) HC/APAP | 16 | 1.32 | 1.75 | B-A | 0.118 |
| C) With NTX 1 | 19 | 0.80 | 2.63 | C-A | 0.328 |
| D) W/NTX 0.1 | 15 | 1.51 | 2.75 | D-A | 0.078 |
| E) W/NTX 0.01 | 19 | 0.94 | 1.32 | E-A | 0.243 |
| F) W/NTX 0.001 | 20 | 2.83 | 2.99 | F-A | <0.001 |
| | | | | C-B | 0.537 |
| | | | | D-B | 0.829 |
| | | | | E-B | 0.657 |
| | | | | F-B | 0.074 |

CATEGORICAL SPID SCORES (6 HOURS)

| | | | | | |
|---|---|---|---|---|---|
| A) Placebo | 22 | −0.47 | 4.36 | TRT | 0.019 |
| B) HC/APAP | 16 | 1.45 | 2.36 | B-A | 0.103 |
| C) With NTX 1 | 19 | 0.43 | 3.24 | C-A | 0.420 |
| D) W/NTX 0.1 | 15 | 1.65 | 3.95 | D-A | 0.077 |
| E) W/NTX 0.01 | 19 | 0.84 | 1.66 | E-A | 0.241 |
| F) W/NTX 0.001 | 20 | 3.43 | 4.48 | F-A | <0.001 |
| | | | | C-B | 0.400 |
| | | | | D-B | 0.874 |
| | | | | E-B | 0.615 |
| | | | | F-B | 0.098 |

CATEGORICAL SPID SCORES (8 HOURS)

| | | | | | |
|---|---|---|---|---|---|
| A) Placebo | 22 | −0.95 | 5.96 | TRT | 0.040 |
| B) HC/APAP | 16 | 1.45 | 2.91 | B-A | 0.115 |
| C) With NTX 1 | 19 | 0.01 | 3.90 | C-A | 0.507 |
| D) W/NTX 0.1 | 15 | 1.78 | 5.26 | D-A | 0.078 |
| E) W/NTX 0.01 | 19 | 0.73 | 2.05 | E-A | 0.243 |
| F) W/NTX 0.001 | 20 | 3.63 | 5.59 | F-A | 0.002 |
| | | | | C-B | 0.357 |
| | | | | D-B | 0.839 |
| | | | | E-B | 0.648 |
| | | | | F-B | 0.158 |

MEANS GIVEN ARE LEAST SQUARE MEANS.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

Tables 70A for females and 70B for males summarize the results of the time to onset of analgesia. In females, the 0.1 mg NTX and the 0.001 mg NTX combination groups had the shortest median times to onset of analgesia. In males, the placebo, HC/APAP alone, and 0.001 mg NTX combination groups had the shortest median 5: times to onset of analgesia. In females, the 0.1 mg NTX and the 0.001 mg NTX combination groups had the highest percentage of patients with analgesia. All active treatment groups had a higher percentage of patients with analgesia than the placebo group. In males, the 0.001 mg NTX combination group had the highest percentage of patients with analgesia.

TABLE 70A
Efficacy Results - Results of Time to Analyses and Percent of Patients with Events
(Safety Patients) Female Patients
TIME TO ONSET OF ANALGESIA (hours)

| | | | 95% INTERVAL | | | |
|---|---|---|---|---|---|---|
| | NUMBER OF | MEDIAN | LOWER | UPPER | | |
| TREATMENT | PATIENTS | TIME | LIMIT | LIMIT | SOURCE | P-VALUE |
| A) Placebo | 28 | >0.8 | 0.5 | >8.0 | TRT | 0.061 |
| B) HC/APAP | 34 | 0.8 | 0.5 | 1.5 | B-A | 0.143 |
| C) W/NTX 1 | 31 | 0.8 | 0.5 | 0.8 | C-A | 0.116 |
| D) W/NTX 0.1 | 35 | 0.5 | 0.5 | 0.8 | D-A | 0.016 |
| E) W/NTX 0.01 | 31 | 1.3 | 0.8 | >8.0 | E-A | 0.744 |
| F) W/NTX 0.001 | 30 | 0.5 | 0.5 | 1.0 | F-A | 0.048 |
| TOTAL | 189 | 0.8 | 0.5 | 1.0 | C-B | 0.707 |
| | | | | | D-B | 0.211 |
| | | | | | E-B | 0.232 |
| | | | | | F-B | 0.470 |

| PATIENTS WITH ANALGESIA | NO | YES | SOURCE | P-VALUE |
|---|---|---|---|---|
| A) Placebo | 15 (54%) | 13 (46%) | TRT | 0.015 |
| B) HC/APAP | 10 (29%) | 24 (71%) | B-A | 0.053 |
| C) W/NTX 1 | 7 (23%) | 24 (77%) | C-A | 0.013 |
| D) W/NTX 0.1 | 6 (17%) | 29 (83%) | D-A | 0.002 |
| E) W/NTX 0.01 | 13 (42%) | 18 (58%) | E-A | 0.371 |
| F) W/NTX 0.001 | 6 (20%) | 24 (80%) | F-A | 0.007 |
| TOTAL | 57 (30%) | 132 (70%) | C-B | 0.530 |
| | | | D-B | 0.226 |
| | | | E-B | 0.291 |
| | | | F-B | 0.383 |

P-VALUES FOR PERCENT OF PATIENTS WITH EVENT ARE FROM THE LIKELIHOOD-RATIO CHI-SQUARE TEST.
P-VALUES FOR TIME TO EVENT ARE FROM THE LONG RANK TEST.

TABLE 70B
Efficacy Results - Results of Time to Analyses and Percent of Patients with Events
(Safety Patients) Male Patients
TIME TO ONSET OF ANALGESIA (hours)

| | | | 95% INTERVAL | | | |
|---|---|---|---|---|---|---|
| | NUMBER OF | MEDIAN | LOWER | UPPER | | |
| TREATMENT | PATIENTS | TIME | LIMIT | LIMIT | SOURCE | P-VALUE |
| A) Placebo | 22 | 0.5 | 0.5 | >8.0 | TRT | 0.237 |
| B) HC/APAP | 16 | 0.5 | 0.5 | 1.0 | B-A | 0.624 |
| C) W/NTX 1 | 19 | 0.8 | 0.5 | >8.0 | C-A | 0.832 |
| D) W/NTX 0.1 | 15 | 0.8 | 0.5 | >8.0 | D-A | 0.735 |
| E) W/NTX 0.01 | 19 | 0.8 | 0.5 | 1.5 | E-A | 0.934 |
| F) W/NTX 0.001 | 90 | 0.5 | 0.3 | 0.8 | F-A | 0.119 |
| TOTAL | 111 | 0.5 | 0.5 | 0.8 | C-B | 0.427 |
| | | | | | D-B | 0.383 |
| | | | | | E-B | 0.526 |
| | | | | | F-B | 0.210 |

| PATIENTS WITH ANALGESIA | NO | YES | SOURCE | P-VALUE |
|---|---|---|---|---|
| A) Placebo | 8 (36%) | 14 (64%) | TRT | 0.087 |
| B) HC/APAP | 3 (19%) | 13 (81%) | B-A | 0.296 |
| C) W/NTX 1 | 7 (37%) | 12 (63%) | C-A | 1.000 |
| D) W/NTX 0.1 | 6 (40%) | 9 (60%) | D-A | 1.000 |
| E) W/NTX 0.01 | 5 (26%) | 14 (74%) | E-A | 0.524 |
| F) W/NTX 0.001 | 1 (5%) | 19 (95%) | F-A | 0.022 |
| TOTAL | 30 (27%) | 81 (73%) | C-B | 0.285 |
| | | | D-B | 0.252 |
| | | | E-B | 0.700 |
| | | | F-B | 0.303 |

P-VALUES FOR PERCENT OF PATIENTS WITH EVENT ARE FROM THE LIKELIHOOD-RATIO CHI-SQUARE TEST.
P-VALUES FOR TIME TO EVENT ARE FROM THE LONG RANK TEST.

Tables 71A for females and 71B for males summarize the results of the time to onset of meaningful pain relief. In females, the time to onset of relief was shortest in the 0.1 mg NTX and the 0.001 mg NTX combination groups. In males, the time to onset of relief was shortest in the HC/APAP alone, 0.1 mg NTX and the 0.001 mg NTX combination groups. In females, the 0.001 mg NTX combination group had the highest percentage of patients reporting relief. In males, the placebo group had the lowest percentage of patients reporting relief and the 0.001 mg NTX combination group had the highest percentage reporting relief.

TABLE 71A
Efficacy Results - Results of Time Onset of Meaningful Pain Relief
(Safety Patients) Female Patients
TIME TO ONSET OF RELIEF (hours)

| | | | 95% INTERVAL | | | |
|---|---|---|---|---|---|---|
| | NUMBER OF | MEDIAN | LOWER | UPPER | | |
| TREATMENT | PATIENTS | TIME | LIMIT | LIMIT | SOURCE | P-VALUE |
| A) Placebo | 28 | >8.0 | 0.8 | >8.0 | TRT | 0.302 |
| B) HC/APAP | 34 | >8.0 | 1.0 | >8.0 | B-A | 0.806 |
| C) W/NTX 1 | 31 | >8.0 | 0.8 | >8.0 | C-A | 0.988 |
| D) W/NTX 0.1 | 35 | 0.9 | 0.5 | >8.0 | D-A | 0.391 |
| E) W/NTX 0.01 | 31 | >8.0 | 1.3 | >8.0 | E-A | 0.336 |
| F) W/NTX 0.001 | 30 | 1.0 | 0.5 | >8.0 | F-A | 0.341 |
| TOTAL | 189 | 2.0 | 1.1 | >8.0 | C-B | 0.730 |
| | | | | | D-B | 0.185 |
| | | | | | E-B | 0.473 |
| | | | | | F-B | 0.133 |

| PATIENTS WITH RELIEF | NO | YES | SOURCE | P-VALUE |
|---|---|---|---|---|
| A) Placebo | 15 (54%) | 13 (46%) | TRT | 0.378 |
| B) HC/APAP | 18 (53%) | 16 (47%) | B-A | 0.961 |
| C) W/NTX 1 | 15 (48%) | 16 (52%) | C-A | 0.691 |
| D) W/NTX 0.1 | 14 (40%) | 21 (60%) | D-A | 0.282 |
| E) W/NTX 0.01 | 19 (61%) | 12 (39%) | E-A | 0.549 |
| F) W/NTX 0.001 | 11 (37%) | 19 (63%) | F-A | 0.195 |
| TOTAL | 92 (49%) | 97 (51%) | C-B | 0.714 |
| | | | D-B | 0.281 |
| | | | E-B | 0.497 |
| | | | F-B | 0.190 |

P-VALUES FOR PERCENT OF PATIENTS WITH EVENT ARE FROM THE LIKELIHOOD-RATIO CHI-SQUARE TEST.
P-VALUES FOR TIME TO EVENT ARE FROM THE LOG RANK TEST.

TABLE 71B
Efficacy Results - Results of Time Onset of Meaningful Pain Relief
(Safety Patients) Male Patients
TIME TO ONSET OF RELIEF (hours)

| | NUMBER | | 95% INTERVAL | | | |
|---|---|---|---|---|---|---|
| | OF | MEDIAN | LOWER | UPPER | | |
| TREATMENT | PATIENTS | TIME | LIMIT | LIMIT | SOURCE | P-VALUE |
| A) Placebo | 22 | >8.0 | 0.8 | >8.0 | TRT | 0.018 |
| B) HC/APAP | 16 | 0.7 | 0.5 | >8.0 | B-A | 0.023 |
| C) W/NTX 1 | 19 | >8.0 | 0.4 | >8.0 | C-A | 0.153 |
| D) W/NTX 0.1 | 15 | 0.7 | 0.3 | >8.0 | D-A | 0.008 |
| E) W/NTX 0.01 | 19 | >8.0 | 1.1 | >8.0 | E-A | 0.781 |
| F) W/NTX 0.001 | 20 | 0.7 | 0.5 | >8.0 | F-A | 0.005 |
| TOTAL | 111 | >8.0 | 0.8 | >8.0 | C-B | 0.488 |
| | | | | | D-B | 0.756 |
| | | | | | E-B | 0.041 |
| | | | | | F-B | 0.744 |

| PATIENTS WITH RELIEF | NO | YES | SOURCE | P-VALUE |
|---|---|---|---|---|
| A) Placebo | 16 (73%) | 6 (27%) | TRT | 0.020 |
| B) HC/APAP | 6 (38%) | 10 (63%) | B-A | 0.029 |
| C) W/NTX 1 | 10 (53%) | 9 (47%) | C-A | 0.182 |
| D) W/NTX 0.1 | 5 (33%) | 10 (67%) | D-A | 0.017 |
| E) W/NTX 0.01 | 13 (68%) | 6 (32%) | E-A | 0.763 |
| F) W/NTX 0.001 | 6 (30%) | 14 (70%) | F-A | 0.005 |
| TOTAL | 56 (50%) | 55 (50%) | C-B | 0.369 |
| | | | D-B | 0.808 |
| | | | E-B | 0.065 |
| | | | F-B | 0.636 |

P-VALUES FOR PERCENT OF PATIENTS WITH EVENT ARE FROM THE LIKELIHOOD-RATIO CHI-SQUARE TEST.
P-VALUES FOR TIME TO EVENT ARE FROM THE LOG RANK TEST.

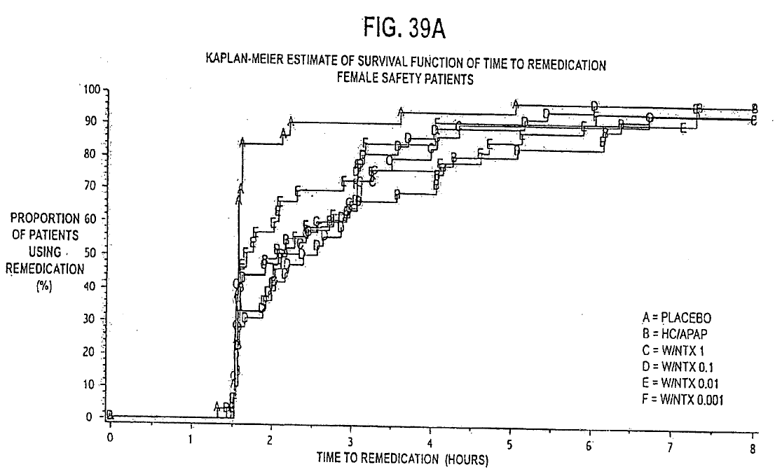

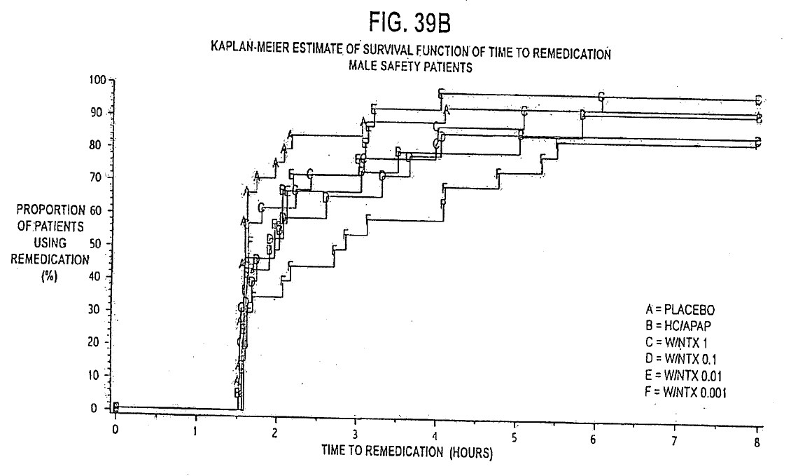

Tables 72A for females and 72B for males summarize the results of the time to remedication (see also FIGS. 39A for females and 39B for males). In females, the placebo group had the shortest median time to remedication and the 0.1 mg NTX treatment group had the longest median time to remedication. In males, the placebo and 1.0 mg NTX combination groups had the shortest median times to remedication and the 0.001 mg NTX combination group had the longest median time to remedication.

Tables 73A for females and 73B for males summarize the results of the percent of patients remedicating. In females, the percentage of patients remedicating was comparable across all treatment groups. In males, the 0.1 mg NTX and the 0.001 mg NTX combination groups had the lowest percentages of patients remedicating.

TABLE 72A
Efficacy Results - Time to Rescue Medication
(Safety Patients) Female Patients
TIME TO REMEDICATION (hours)

| | NUMBER | | 95% INTERVAL | | | |
|---|---|---|---|---|---|---|
| | OF | MEDIAN | LOWER | UPPER | | |
| TREATMENT | PATIENTS | TIME | LIMIT | LIMIT | SOURCE | P-VALUE |
| A) Placebo | 28 | 1.6 | 1.6 | 1.6 | TRT | 0.002 |
| B) HC/APAP | 34 | 1.9 | 1.6 | 3.1 | B-A | <0.001 |
| C) W/NTX 1 | 31 | 2.0 | 1.6 | 3.0 | C-A | 0.011 |
| D) W/NTX 0.1 | 35 | 2.3 | 1.9 | 3.1 | D-A | <0.001 |
| E) W/NTX 0.01 | 31 | 1.7 | 1.6 | 2.1 | E-A | 0.011 |
| F) W/NTX 0.001 | 30 | 2.1 | 1.6 | 3.1 | F-A | 0.002 |
| TOTAL | 189 | 1.9 | 1.6 | 2.1 | C-B | 0.664 |
| | | | | | D-B | 0.218 |
| | | | | | E-B | 0.525 |
| | | | | | F-B | 0.523 |

P-VALUES FOR PERCENT OF PATIENTS WITH EVENT ARE FROM FISHER'S EXACT TEST.

TABLE 72B
Efficacy Results - Time to Rescue Medication
(Safety Patients) Male Patients
TIME TO REMEDICATION (hours)

| | NUMBER | | 95% INTERVAL | | | |
|---|---|---|---|---|---|---|
| | OF | MEDIAN | LOWER | UPPER | | |
| TREATMENT | PATIENTS | TIME | LIMIT | LIMIT | SOURCE | P-VALUE |
| A) Placebo | 22 | 1.6 | 1.6 | 1.7 | TRT | 0.040 |
| B) HC/APAP | 16 | 1.9 | 1.6 | 3.1 | B-A | 0.121 |
| C) W/NTX 1 | 19 | 1.6 | 1.6 | 2.4 | C-A | 0.338 |
| D) W/NTX 0.1 | 15 | 1.8 | 1.6 | 3.7 | D-A | 0.066 |
| E) W/NTX 0.01 | 19 | 1.7 | 1.6 | 2.2 | E-A | 0.385 |
| F) W/NTX 0.001 | 20 | 2.7 | 1.7 | 4.8 | F-A | 0.007 |
| TOTAL | 111 | 1.7 | 1.6 | 2.1 | C-B | 0.508 |
| | | | | | D-B | 0.659 |
| | | | | | E-B | 0.288 |
| | | | | | F-B | 0.283 |

P-VALUES FOR TIME TO EVENT ARE FROM THE LOG RANK TEST.

TABLE 73A
Efficacy Results Percent of Patients Remedicating
Intent-To-Treat Population, Female Patients
PATIENTS REMEDICATING

| TREATMENT | NO | YES | SOURCE | P-VALUE |
|---|---|---|---|---|
| A) Placebo | 0 (0%) | 28 (100%) | TRT | 0.314 |
| B) HC/APAP | 0 (0%) | 34 (100%) | B-A | 0.314 |
| C) W/NTX1 | 2 (6%) | 29 (94%) | C-A | 0.493 |
| D) W/NTX 0.1 | 0 (0%) | 35 (100%) | D-A | 0.493 |
| E) W/NTX 0.01 | 1 (3%) | 30 (97%) | E-A | 1.000 |
| F) W/NTX 0.001 | 1 (3%) | 29 (97%) | F-A | 1.000 |
| TOTAL | 4 (2%) | 185 (98%) | C-B | 0.224 |
| | | | D-B | 0.224 |
| | | | E-B | 0.477 |
| | | | F-B | 0.469 |

P-VALUES FOR PERCENT OF PATIENTS WITH EVENT ARE FROM FISHER'S EXACT TEST.

TABLE 73B
Efficacy Results Percent of Patients Remedicating
Intent-To-Treat Population, Male Patients
PATIENTS REMEDICATING

| TREATMENT | NO | YES | SOURCE | P-VALUE |
|---|---|---|---|---|
| A) Placebo | 1 (5%) | 21 (95%) | TRT | 0.222 |
| B) HC/APAP | 1 (6%) | 15 (94%) | B-A | 1.000 |
| C) W/NTX 1 | 0 (0%) | 19 (100%) | C-A | 1.000 |
| D) W/NTX 0.1 | 2 (13%) | 13 (87%) | D-A | 0.554 |
| E) W/NTX 0.01 | 0 (0%) | 19 (100%) | E-A | 1.000 |
| F) W/NTX 0.001 | 3 (15%) | 17 (85%) | F-A | 0.333 |
| TOTAL | 7 (6%) | 104 (94%) | C-B | 0.457 |
| | | | D-B | 0.600 |
| | | | E-B | 0.457 |
| | | | F-B | 0.613 |

P-VALUES FOR PERCENT OF PATIENTS WITH EVENT ARE FROM FISHER'S EXACT TEST.

Tables 74A for females and 74B for males summarize the results of the pain relief (PR) scores, and Tables 74C for females and 74D for males summarize the MAXPAR scores. In females, the placebo group had the lowest mean pain relief scores from 30 minutes to 5 hours. In males, the 0.001 mg NTX combination group had the highest mean pain relief scores from 15 minutes to 8 hours. In females, the 1.0 mg NTX and the 0.001 mg NTX combination groups had the highest mean peak relief scores. In males, the 0.001 mg NTX combination group had the highest mean peak relief scores.

TABLE 74A
Efficacy Results - Means and Standard Deviations
for the Pain Relief Scores (Safety Patients)
Female Patients

| PAIN RELIEF SCORES | | | | | |
|---|---|---|---|---|---|
| TREATMENT | N | MEAN | SD | SOURCE | P-VALUE |
| 15 MINUTES | | | | | |
| A) Placebo | 28 | 0.61 | 0.96 | TRT | 0.440 |
| B) HC/APAP | 34 | 0.44 | 0.66 | B-A | 0.447 |
| C) W/NTX 1 | 31 | 0.65 | 0.91 | C-A | 0.864 |
| D) W/NTX 0.1 | 35 | 0.77 | 1.14 | D-A | 0.448 |
| E) W/NTX 0.01 | 31 | 0.39 | 0.62 | E-A | 0.324 |
| F) W/NTX 0.001 | 30 | 0.47 | 0.68 | F-A | 0.532 |
| | | | | C-B | 0.337 |
| | | | | D-B | 0.110 |
| | | | | E-B | 0.799 |
| | | | | F-B | 0.905 |
| 30 MINUTES | | | | | |
| A) Placebo | 28 | 0.79 | 1.03 | TRT | 0.054 |
| B) HC/APAP | 34 | 1.02 | 1.08 | B-A | 0.423 |
| C) W/NTX 1 | 31 | 1.42 | 1.18 | C-A | 0.035 |
| D) W/NTX 0.1 | 35 | 1.50 | 1.22 | D-A | 0.015 |
| E) W/NTX 0.01 | 31 | 1.03 | 1.20 | E-A | 0.410 |
| F) W/NTX 0.001 | 30 | 1.53 | 1.14 | F-A | 0.014 |
| | | | | C-B | 0.162 |
| | | | | D-B | 0.086 |
| | | | | E-B | 0.966 |
| | | | | F-B | 0.075 |
| 45 MINUTES | | | | | |
| A) Placebo | 28 | 0.89 | 0.99 | TRT | 0.008 |
| B) HC/APAP | 34 | 1.56 | 1.19 | B-A | 0.021 |
| C) W/NTX 1 | 31 | 1.76 | 1.12 | C-A | 0.003 |
| D) W/NTX 0.1 | 35 | 1.91 | 1.20 | D-A | <0.001 |
| E) W/NTX 0.01 | 31 | 1.35 | 1.02 | E-A | 0.116 |
| F) W/NTX 0.001 | 30 | 1.73 | 1.17 | F-A | 0.005 |
| | | | | C-B | 0.466 |
| | | | | D-B | 0.190 |
| | | | | E-B | 0.465 |
| | | | | F-B | 0.535 |
| 1 HOUR | | | | | |
| A) Placebo | 28 | 0.82 | 1.12 | TRT | <0.001 |
| B) HC/APAP | 34 | 1.73 | 1.17 | B-A | 0.004 |
| C) W/NTX 1 | 31 | 1.94 | 1.34 | C-A | <0.001 |
| D) W/NTX 0.1 | 35 | 2.00 | 1.21 | D-A | <0.001 |
| E) W/NTX 0.01 | 31 | 1.48 | 1.31 | E-A | 0.040 |
| F) W/NTX 0.001 | 30 | 2.10 | 1.18 | F-A | <0.001 |
| | | | | C-B | 0.492 |
| | | | | D-B | 0.354 |
| | | | | E-B | 0.429 |
| | | | | F-B | 0.225 |
| 1.5 HOURS | | | | | |
| A) Placebo | 28 | 0.57 | 0.96 | TRT | 0.001 |
| B) HC/APAP | 34 | 1.65 | 1.35 | B-A | 0.001 |
| C) W/NTX 1 | 31 | 1.81 | 1.47 | C-A | <0.001 |
| D) W/NTX 0.1 | 35 | 1.69 | 1.21 | D-A | <0.001 |
| E) W/NTX 0.01 | 31 | 1.55 | 1.34 | E-A | 0.003 |
| F) W/NTX 0.001 | 30 | 1.93 | 1.17 | F-A | <0.001 |
| | | | | C-B | 0.612 |
| | | | | D-B | 0.899 |
| | | | | E-B | 0.754 |
| | | | | F-B | 0.367 |
| 2 HOURS | | | | | |
| A) Placebo | 28 | 0.21 | 0.79 | TRT | 0.009 |
| B) HC/APAP | 34 | 1.41 | 1.50 | B-A | <0.001 |
| C) W/NTX 1 | 31 | 1.35 | 1.59 | C-A | 0.002 |
| D) W/NTX 0.1 | 35 | 1.29 | 1.36 | D-A | 0.002 |
| E) W/NTX 0.01 | 31 | 1.00 | 1.41 | E-A | 0.027 |
| F) W/NTX 0.001 | 30 | 1.23 | 1.25 | F-A | 0.005 |
| | | | | C-B | 0.844 |
| | | | | D-B | 0.699 |
| | | | | E-B | 0.222 |
| | | | | F-B | 0.599 |
| 3 HOURS | | | | | |
| A) Placebo | 28 | 0.18 | 0.67 | TRT | 0.211 |
| B) HC/APAP | 34 | 0.91 | 1.33 | B-A | 0.012 |
| C) W/NTX 1 | 31 | 0.71 | 1.25 | C-A | 0.069 |
| D) W/NTX 0.1 | 35 | 0.60 | 1.03 | D-A | 0.142 |
| E) W/NTX 0.01 | 31 | 0.68 | 1.30 | E-A | 0.091 |
| F) W/NTX 0.001 | 30 | 0.50 | 0.97 | F-A | 0.279 |
| | | | | C-B | 0.482 |
| | | | | D-B | 0.252 |
| | | | | E-B | 0.403 |
| | | | | F-B | 0.146 |
| 4 HOURS | | | | | |
| A) Placebo | 28 | 0.11 | 0.57 | TRT | 0.199 |
| B) HC/APAP | 34 | 0.71 | 1.31 | B-A | 0.021 |
| C) W/NTX 1 | 30 | 0.39 | 0.99 | C-A | 0.281 |
| D) W/NTX 0.1 | 35 | 0.29 | 0.86 | D-A | 0.486 |
| E) W/NTX 0.01 | 31 | 0.61 | 1.20 | E-A | 0.056 |
| F) W/NTX 0.001 | 30 | 0.33 | 0.88 | F-A | 0.395 |
| | | | | C-B | 0.220 |
| | | | | D-B | 0.086 |
| | | | | E-B | 0.711 |
| | | | | F-B | 0.143 |
| 5 HOURS | | | | | |
| A) Placebo | 28 | 0.04 | 0.19 | TRT | 0.406 |
| B) HC/APAP | 34 | 0.47 | 1.16 | B-A | 0.043 |
| C) W/NTX 1 | 30 | 0.23 | 0.90 | C-A | 0.370 |
| D) W/NTX 0.1 | 35 | 0.20 | 0.68 | D-A | 0.440 |
| E) W/NTX 0.01 | 31 | 0.35 | 1.02 | E-A | 0.146 |
| F) W/NTX 0.001 | 30 | 0.17 | 0.65 | F-A | 0.553 |
| | | | | C-B | 0.260 |
| | | | | D-B | 0.181 |
| | | | | E-B | 0.579 |
| | | | | F-B | 0.149 |
| 6 HOURS | | | | | |
| A) Placebo | 28 | 0.00 | 0.00 | TRT | 0.239 |
| B) HC/APAP | 34 | 0.38 | 1.02 | B-A | 0.040 |
| C) W/NTX 1 | 30 | 0.23 | 0.90 | C-A | 0.222 |
| D) W/NTX 0.1 | 35 | 0.00 | 0.00 | D-A | 1.000 |
| E) W/NTX 0.01 | 31 | 0.23 | 0.80 | E-A | 0.234 |
| F) W/NTX 0.001 | 30 | 0.20 | 0.81 | F-A | 0.295 |
| | | | | C-B | 0.413 |
| | | | | D-B | 0.030 |
| | | | | E-B | 0.386 |
| | | | | F-B | 0.317 |
| 7 HOURS | | | | | |
| A) Placebo | 28 | 0.00 | 0.00 | TRT | 0.639 |
| B) HC/APAP | 34 | 0.06 | 0.34 | B-A | 0.592 |
| C) W/NTX 1 | 30 | 0.10 | 0.55 | C-A | 0.376 |
| D) W/NTX 0.1 | 35 | 0.00 | 0.00 | D-A | 1.000 |
| E) W/NTX 0.01 | 31 | 0.16 | 0.64 | E-A | 0.151 |
| F) W/NTX 0.001 | 30 | 0.10 | 0.55 | F-A | 0.376 |
| | | | | C-B | 0.702 |
| | | | | D-B | 0.570 |
| | | | | E-B | 0.337 |
| | | | | F-B | 0.702 |
| 8 HOURS | | | | | |
| A) Placebo | 28 | 0.00 | 0.00 | TRT | 0.518 |
| B) HC/APAP | 34 | 0.00 | 0.00 | B-A | 1.000 |
| C) W/NTX 1 | 30 | 0.10 | 0.55 | C-A | 0.221 |
| D) W/NTX 0.1 | 35 | 0.00 | 0.00 | D-A | 1.000 |
| E) W/NTX 0.01 | 30 | 0.00 | 0.00 | E-A | 1.000 |
| F) W/NTX 0.001 | 30 | 0.10 | 0.55 | F-A | 0.221 |
| | | | | C-B | 0.200 |
| | | | | D-B | 1.000 |
| | | | | E-B | 1.000 |
| | | | | F-B | 0.200 |

MEANS GIVEN ARE LEAST SQUARE MEANS.
THE PAIN RELIEF SCALE WAS: 0 = NONE, 1 = A LITTLE, 2 = SOME, 3 = A LOT, AND 4 = COMPLETE.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE 74B
Efficacy Results - Means and Standard Deviations
for the Pain Relief Scores (Safety Patients)
Male Patients

| PAIN RELIEF SCORES | | | | | |
|---|---|---|---|---|---|
| TREATMENT | N | MEAN | SD | SOURCE | P-VALUE |
| 15 MINUTES | | | | | |
| A) Placebo | 22 | 0.68 | 0.78 | TRT | 0.307 |
| B) HC/APAP | 16 | 0.38 | 0.62 | B-A | 0.206 |
| C) W/NTX 1 | 19 | 0.47 | 0.84 | C-A | 0.367 |
| D) W/NTX 0.1 | 15 | 0.53 | 0.74 | D-A | 0.547 |
| E) W/NTX 0.01 | 19 | 0.26 | 0.56 | E-A | 0.071 |
| F) W/NTX 0.001 | 20 | 0.75 | 0.79 | F-A | 0.764 |
| | | | | C-B | 0.692 |
| | | | | D-B | 0.549 |
| | | | | E-B | 0.654 |
| | | | | F-B | 0.130 |
| 30 MINUTES | | | | | |
| A) Placebo | 22 | 0.91 | 1.06 | TRT | 0.013 |
| B) HC/APAP | 16 | 1.13 | 1.09 | B-A | 0.535 |
| C) W/NTX 1 | 19 | 1.32 | 1.25 | C-A | 0.222 |
| D) W/NTX 0.1 | 15 | 0.99 | 0.78 | D-A | 0.825 |
| E) W/NTX 0.01 | 19 | 0.63 | 0.90 | E-A | 0.403 |
| F) W/NTX 0.001 | 20 | 1.85 | 1.14 | F-A | 0.005 |
| | | | | C-B | 0.596 |
| | | | | D-B | 0.718 |
| | | | | E-B | 0.171 |
| | | | | F-B | 0.043 |
| 45 MINUTES | | | | | |
| A) Placebo | 22 | 0.95 | 1.05 | TRT | 0.005 |
| B) HC/APAP | 16 | 1.44 | 0.96 | B-A | 0.171 |
| C) W/NTX 1 | 19 | 1.63 | 1.21 | C-A | 0.045 |
| D) W/NTX 0.1 | 15 | 1.66 | 1.15 | D-A | 0.051 |
| E) W/NTX 0.01 | 19 | 1.26 | 0.99 | E-A | 0.357 |
| F) W/NTX 0.001 | 20 | 2.27 | 1.02 | F-A | <0.001 |
| | | | | C-B | 0.593 |
| | | | | D-B | 0.562 |
| | | | | E-B | 0.631 |
| | | | | F-B | 0.022 |
| 1 HOUR | | | | | |
| A) Placebo | 22 | 1.05 | 1.17 | TRT | 0.030 |
| B) HC/APAP | 16 | 1.63 | 0.81 | B-A | 0.148 |
| C) W/NTX 1 | 19 | 1.37 | 1.16 | C-A | 0.396 |
| D) W/NTX 0.1 | 15 | 1.86 | 1.45 | D-A | 0.046 |
| E) W/NTX 0.01 | 19 | 1.76 | 1.27 | E-A | 0.061 |
| F) W/NTX 0.001 | 20 | 2.30 | 1.30 | F-A | 0.001 |
| | | | | C-B | 0.533 |
| | | | | D-B | 0.585 |
| | | | | E-B | 0.737 |
| | | | | F-B | 0.099 |
| 1.5 HOURS | | | | | |
| A) Placebo | 22 | 0.86 | 0.94 | TRT | 0.009 |
| B) HC/APAP | 16 | 1.56 | 1.21 | B-A | 0.094 |
| C) W/NTX 1 | 19 | 1.05 | 1.18 | C-A | 0.632 |
| D) W/NTX 0.1 | 15 | 1.53 | 1.46 | D-A | 0.115 |
| E) W/NTX 0.01 | 19 | 1.63 | 1.30 | E-A | 0.054 |
| F) W/NTX 0.001 | 20 | 2.30 | 1.45 | F-A | <0.001 |
| | | | | C-B | 0.235 |
| | | | | D-B | 0.949 |
| | | | | E-B | 0.872 |
| | | | | F-B | 0.083 |
| 2 HOURS | | | | | |
| A) Placebo | 22 | 0.45 | 1.06 | TRT | 0.036 |
| B) HC/APAP | 16 | 1.06 | 1.53 | B-A | 0.186 |
| C) W/NTX 1 | 19 | 0.95 | 1.39 | C-A | 0.260 |
| D) W/NTX 0.1 | 15 | 1.27 | 1.44 | D-A | 0.084 |
| E) W/NTX 0.01 | 19 | 0.84 | 1.26 | E-A | 0.375 |
| F) W/NTX 0.001 | 20 | 1.90 | 1.65 | F-A | 0.001 |
| | | | | C-B | 0.807 |
| | | | | D-B | 0.683 |
| | | | | E-B | 0.641 |
| | | | | F-B | 0.075 |
| 3 HOURS | | | | | |
| A) Placebo | 22 | 0.27 | 0.94 | TRT | 0.033 |
| B) HC/APAP | 16 | 0.56 | 1.15 | B-A | 0.465 |
| C) W/NTX 1 | 19 | 0.68 | 1.25 | C-A | 0.277 |
| D) W/NTX 0.1 | 15 | 0.76 | 1.20 | D-A | 0.225 |
| E) W/NTX 0.01 | 19 | 0.32 | 0.75 | E-A | 0.909 |
| F) W/NTX 0.001 | 20 | 1.45 | 1.70 | F-A | 0.002 |
| | | | | C-B | 0.766 |
| | | | | D-B | 0.642 |
| | | | | E-B | 0.547 |
| | | | | F-B | 0.030 |
| 4 HOURS | | | | | |
| A) Placebo | 22 | 0.18 | 0.85 | TRT | 0.023 |
| B) HC/APAP | 16 | 0.50 | 1.10 | B-A | 0.377 |
| C) W/NTX 1 | 19 | 0.32 | 0.95 | C-A | 0.696 |
| D) W/NTX 0.1 | 15 | 0.40 | 1.06 | D-A | 0.552 |
| E) W/NTX 0.01 | 19 | 0.05 | 0.23 | E-A | 0.706 |
| F) W/NTX 0.001 | 20 | 1.20 | 1.77 | F-A | 0.003 |
| | | | | C-B | 0.620 |
| | | | | D-B | 0.799 |
| | | | | E-B | 0.230 |
| | | | | F-B | 0.059 |
| 5 HOURS | | | | | |
| A) Placebo | 22 | 0.14 | 0.64 | TRT | 0.064 |
| B) HC/APAP | 16 | 0.38 | 0.89 | B-A | 0.427 |
| C) W/NTX 1 | 19 | 0.16 | 0.50 | C-A | 0.940 |
| D) W/NTX 0.1 | 15 | 0.40 | 1.06 | D-A | 0.389 |
| E) W/NTX 0.01 | 19 | 0.00 | 0.00 | E-A | 0.633 |
| F) W/NTX 0.001 | 20 | 0.85 | 1.57 | F-A | 0.013 |
| | | | | C-B | 0.484 |
| | | | | D-B | 0.939 |
| | | | | E-B | 0.227 |
| | | | | F-B | 0.123 |
| 6 HOURS | | | | | |
| A) Placebo | 22 | 0.18 | 0.85 | TRT | 0.342 |
| B) HC/APAP | 16 | 0.19 | 0.54 | B-A | 0.983 |
| C) W/NTX 1 | 19 | 0.05 | 0.23 | C-A | 0.602 |
| D) W/NTX 0.1 | 15 | 0.40 | 1.06 | D-A | 0.410 |
| E) W/NTX 0.01 | 19 | 0.00 | 0.00 | E-A | 0.463 |
| F) W/NTX 0.001 | 20 | 0.50 | 1.24 | F-A | 0.194 |
| | | | | C-B | 0.615 |
| | | | | D-B | 0.455 |
| | | | | E-B | 0.485 |
| | | | | F-B | 0.240 |
| 7 HOURS | | | | | |
| A) Placebo | 22 | 0.18 | 0.85 | TRT | 0.228 |
| B) HC/APAP | 16 | 0.13 | 0.50 | B-A | 0.832 |
| C) W/NTX 1 | 19 | 0.00 | 0.00 | C-A | 0.477 |
| D) W/NTX 0.1 | 15 | 0.40 | 1.06 | D-A | 0.425 |
| E) W/NTX 0.01 | 19 | 0.00 | 0.00 | E-A | 0.477 |
| F) W/NTX 0.001 | 20 | 0.55 | 1.36 | F-A | 0.146 |
| | | | | C-B | 0.652 |
| | | | | D-B | 0.349 |
| | | | | E-B | 0.652 |
| | | | | F-B | 0.123 |
| 8 HOURS | | | | | |
| A) Placebo | 22 | 0.14 | 0.64 | TRT | 0.214 |
| B) HC/APAP | 16 | 0.19 | 0.75 | B-A | 0.847 |
| C) W/NTX 1 | 19 | 0.00 | 0.00 | C-A | 0.588 |
| D) W/NTX 0.1 | 15 | 0.40 | 1.06 | D-A | 0.329 |
| E) W/NTX 0.01 | 19 | 0.00 | 0.00 | E-A | 0.588 |
| F) W/NTX 0.001 | 20 | 0.55 | 1.36 | F-A | 0.098 |
| | | | | C-B | 0.492 |
| | | | | D-B | 0.463 |
| | | | | E-B | 0.492 |
| | | | | F-B | 0.181 |

MEANS GIVEN ARE LEAST SQUARE MEANS.
THE PAIN RELIEF SCALE WAS: 0 = NONE, 1 = A LITTLE, 2 = SOME, 3 = A LOT, AND 4 = COMPLETE.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE 74C
Efficacy Results - Means and Standard Deviations for MAXPAR
(Safety Patients) Female Patients

| MAXPAR SCORES | | | | | |
|---|---|---|---|---|---|
| TREATMENT | | | | | |
| PEAK RELIEF | N | MEAN | SD | SOURCE | P-VALUE |
| A) Placebo | 28 | 1.36 | 1.31 | TRT | 0.010 |
| B) HC/APAP | 34 | 2.12 | 1.23 | B-A | 0.015 |
| C) W/NTX 1 | 31 | 2.40 | 1.18 | C-A | 0.001 |
| D) W/NTX 0.1 | 35 | 2.29 | 1.15 | D-A | 0.003 |
| E) W/NTX 0.01 | 31 | 1.90 | 1.30 | E-A | 0.085 |
| F) W/NTX 0.001 | 30 | 2.37 | 1.10 | F-A | 0.002 |
| | | | | C-B | 0.341 |
| | | | | D-B | 0.565 |
| | | | | E-B | 0.477 |
| | | | | F-B | 0.413 |

MEANS GIVEN ARE LEAST SQUARE MEANS.
THE PAIN RELIEF SCALE WAS: 0 = NONE, 1 = A LITTLE, 2 = SOME, 3 = A LOT, AND 4 = COMPLETE.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE74D
Efficacy Results - Means and Standard Deviations for MAXPAR
(Safety Patients) Male Patients

| MAXPAR SCORES | | | | | |
|---|---|---|---|---|---|
| TREATMENT | | | | | |
| PEAK RELIEF | N | MEAN | SD | SOURCE | P-VALUE |
| A) Placebo | 22 | 1.59 | 1.30 | TRT | 0.065 |
| B) HC/APAP | 16 | 2.13 | 0.96 | B-A | 0.179 |
| C) W/NTX 1 | 19 | 1.89 | 1.15 | C-A | 0.422 |
| D) W/NTX 0.1 | 15 | 1.95 | 1.35 | D-A | 0.374 |
| E) W/NTX 0.01 | 19 | 1.89 | 1.24 | E-A | 0.422 |
| F) W/NTX 0.001 | 20 | 2.75 | 1.16 | F-A | 0.002 |
| | | | | C-B | 0.574 |
| | | | | D-B | 0.687 |
| | | | | E-B | 0.574 |
| | | | | F-B | 0.124 |

MEANS GIVEN ARE BEST SQUARE MEANS.
THE PAIN RELIEF SCALE WAS 0 = NONE, 1 = A LITTLE, 2 = SOME, 3 = A LOT, AND 4 = COMPLETE.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

Tables 75A for females and 75B for males summarize the results of the pain intensity difference (PD) scores. In females, the placebo group had the lowest mean PID scores from 45 minutes to 8 hours. All active treatment groups had higher mean PID scores than the placebo group. In males, the placebo group had the lowest mean PID scores from 30 minutes to 8 hours. The 0.001 mg NTX combination group had the highest mean PD scores from 15 minutes to 8 hours.

Tables 75C for females and 75D for males summarize the PEAKPID scores. In females, the placebo group had the lowest PEAKPID score and the 1.0 mg NTX and the 0.001 mg NTX combination groups had the highest PEAKPID scores. In males, the 0.001 mg NTX combination group had the highest PEAKPID score.

TABLE 75A
Efficacy Results - Means and Standard Deviations for the Categorical
PID Scores (Safety Patients)
Female Patients

| CATEGORICAL PID SCORES | | | | | |
|---|---|---|---|---|---|
| TREATMENT | N | MEAN | SD | SOURCE | P-VALUE |
| 15 MINUTES | | | | | |
| A) Placebo | 28 | 0.20 | 0.55 | TRT | 0.561 |
| B) HC/APAP | 34 | 0.06 | 0.60 | B-A | 0.360 |
| C) W/NTX 1 | 31 | 0.03 | 0.48 | C-A | 0.285 |
| D) W/NTX 0.1 | 35 | 0.23 | 0.60 | D-A | 0.829 |
| E) W/NTX 0.01 | 31 | 0.00 | 0.58 | E-A | 0.202 |
| F) W/NTX 0.001 | 30 | 0.08 | 0.70 | F-A | 0.465 |
| | | | | C-B | 0.856 |
| | | | | D-B | 0.232 |
| | | | | E-B | 0.687 |
| | | | | F-B | 0.868 |
| 30 MINUTES | | | | | |
| A) Placebo | 28 | 0.32 | 0.72 | TRT | 0.522 |
| B) HC/APAP | 34 | 0.41 | 0.89 | B-A | 0.652 |
| C) W/NTX 1 | 31 | 0.52 | 0.77 | C-A | 0.341 |
| D) W/NTX 0.1 | 35 | 0.65 | 0.68 | D-A | 0.102 |
| E) W/NTX 0.01 | 31 | 0.32 | 0.70 | E-A | 0.996 |
| F) W/NTX 0.001 | 30 | 0.50 | 0.90 | F-A | 0.386 |
| | | | | C-B | 0.592 |
| | | | | D-B | 0.212 |
| | | | | E-B | 0.647 |
| | | | | F-B | 0.653 |
| 45 MINUTES | | | | | |
| A) Placebo | 28 | 0.18 | 0.90 | TRT | 0.042 |
| B) HC/APAP | 34 | 0.56 | 0.86 | B-A | 0.074 |
| C) W/NTX 1 | 31 | 0.81 | 0.79 | C-A | 0.004 |
| D) W/NTX 0.1 | 35 | 0.80 | 0.72 | D-A | 0.004 |
| E) W/NTX 0.01 | 31 | 0.48 | 0.77 | E-A | 0.160 |
| F) W/NTX 0.001 | 30 | 0.57 | 0.94 | F-A | 0.077 |
| | | | | C-B | 0.231 |
| | | | | D-B | 0.229 |
| | | | | E-B | 0.717 |
| | | | | F-B | 0.970 |
| 1 HOUR | | | | | |
| A) Placebo | 28 | 0.05 | 0.91 | TRT | 0.003 |
| B) HC/APAP | 34 | 0.70 | 0.87 | B-A | 0.004 |
| C) W/NTX 1 | 31 | 0.88 | 0.86 | C-A | <0.001 |
| D) W/NTX 0.1 | 35 | 0.80 | 0.72 | D-A | <0.001 |
| E) W/NTX 0.01 | 31 | 0.58 | 0.85 | E-A | 0.019 |
| F) W/NTX 0.001 | 30 | 0.87 | 1.01 | F-A | <0.001 |
| | | | | C-B | 0.394 |
| | | | | D-B | 0.620 |
| | | | | E-B | 0.593 |
| | | | | F-B | 0.434 |
| 1.5 HOURS | | | | | |
| A) Placebo | 28 | −0.04 | 0.74 | TRT | 0.012 |
| B) HC/APAP | 34 | 0.65 | 0.92 | B-A | 0.003 |
| C) W/NTX 1 | 31 | 0.68 | 1.01 | C-A | 0.002 |
| D) W/NTX 0.1 | 35 | 0.60 | 0.69 | D-A | 0.005 |
| E) W/NTX 0.01 | 31 | 0.52 | 0.89 | E-A | 0.016 |
| F) W/NTX 0.001 | 30 | 0.73 | 0.94 | F-A | <0.001 |
| | | | | C-B | 0.889 |
| | | | | D-B | 0.823 |
| | | | | E-B | 0.547 |
| | | | | F-B | 0.694 |
| 2 HOURS | | | | | |
| A) Placebo | 28 | −0.25 | 0.65 | TRT | 0.010 |
| B) HC/APAP | 34 | 0.56 | 0.93 | B-A | <0.001 |
| C) W/NTX 1 | 31 | 0.41 | 1.07 | C-A | 0.004 |
| D) W/NTX 0.1 | 35 | 0.42 | 0.71 | D-A | 0.003 |
| E) W/NTX 0.01 | 31 | 0.39 | 0.88 | E-A | 0.006 |
| F) W/NTX 0.001 | 30 | 0.37 | 0.93 | F-A | 0.008 |
| | | | | C-B | 0.493 |
| | | | | D-B | 0.505 |
| | | | | E-B | 0.429 |
| | | | | F-B | 0.380 |
| 3 HOURS | | | | | |
| A) Placebo | 28 | −0.25 | 0.59 | TRT | 0.104 |
| B) HC/APAP | 34 | 0.26 | 0.75 | B-A | 0.007 |
| C) W/NTX 1 | 31 | 0.07 | 0.92 | C-A | 0.098 |
| D) W/NTX 0.1 | 35 | 0.08 | 0.51 | D-A | 0.083 |
| E) W/NTX 0.01 | 31 | 0.23 | 0.88 | E-A | 0.014 |
| F) W/NTX 0.001 | 30 | 0.00 | 0.69 | F-A | 0.199 |
| | | | | C-B | 0.289 |
| | | | | D-B | 0.289 |
| | | | | E-B | 0.832 |
| | | | | F-B | 0.154 |
| 4 HOURS | | | | | |
| A) Placebo | 28 | −0.29 | 0.53 | TRT | 0.032 |
| B) HC/APAP | 34 | 0.26 | 0.79 | B-A | 0.002 |
| C) W/NTX 1 | 30 | −0.08 | 0.75 | C-A | 0.257 |
| D) W/NTX 0.1 | 35 | 0.05 | 0.49 | D-A | 0.056 |
| E) W/NTX 0.01 | 31 | 0.16 | 0.82 | E-A | 0.013 |
| F) W/NTX 0.001 | 30 | −0.07 | 0.64 | F-A | 0.223 |
| | | | | C-B | 0.044 |
| | | | | D-B | 0.187 |
| | | | | E-B | 0.542 |
| | | | | F-B | 0.054 |
| 5 HOURS | | | | | |
| A) Placebo | 28 | −0.32 | 0.48 | TRT | 0.040 |
| B) HC/APAP | 34 | 0.15 | 0.70 | B-A | 0.003 |
| C) W/NTX 1 | 30 | −0.17 | 0.65 | C-A | 0.337 |
| D) W/NTX 0.1 | 35 | −0.01 | 0.35 | D-A | 0.046 |
| E) W/NTX 0.01 | 31 | 0.06 | 0.81 | E-A | 0.016 |
| F) W/NTX 0.001 | 30 | −0.13 | 0.57 | F-A | 0.243 |
| | | | | C-B | 0.042 |
| | | | | D-B | 0.288 |
| | | | | E-B | 0.587 |
| | | | | F-B | 0.069 |
| 6 HOURS | | | | | |
| A) Placebo | 28 | −0.32 | 0.48 | TRT | 0.191 |
| B) HC/APAP | 34 | 0.06 | 0.55 | B-A | 0.011 |
| C) W/NTX 1 | 30 | −0.17 | 0.65 | C-A | 0.309 |
| D) W/NTX 0.1 | 35 | −0.10 | 0.29 | D-A | 0.124 |
| E) W/NTX 0.01 | 31 | −0.03 | 0.71 | E-A | 0.056 |
| F) W/NTX 0.001 | 30 | −0.10 | 0.71 | F-A | 0.146 |
| | | | | C-B | 0.121 |
| | | | | D-B | 0.268 |
| | | | | E-B | 0.526 |
| | | | | F-B | 0.273 |
| 7 HOURS | | | | | |
| A) Placebo | 28 | −0.32 | 0.48 | TRT | 0.218 |
| B) HC/APAP | 34 | −0.09 | 0.29 | B-A | 0.048 |
| C) W/NTX 1 | 30 | −0.23 | 0.50 | C-A | 0.466 |
| D) W/NTX 0.1 | 35 | −0.10 | 0.29 | D-A | 0.054 |
| E) W/NTX 0.01 | 31 | −0.06 | 0.57 | E-A | 0.033 |
| F) W/NTX 0.001 | 30 | −0.13 | 0.57 | F-A | 0.121 |
| | | | | C-B | 0.209 |
| | | | | D-B | 0.947 |
| | | | | E-B | 0.835 |
| | | | | F-B | 0.695 |
| 8 HOURS | | | | | |
| A) Placebo | 28 | −0.32 | 0.48 | TRT | 0.243 |
| B) HC/APAP | 34 | −0.09 | 0.29 | B-A | 0.033 |
| C) W/NTX 1 | 30 | −0.23 | 0.50 | C-A | 0.431 |
| D) W/NTX 0.1 | 35 | −0.10 | 0.29 | D-A | 0.037 |
| E) W/NTX 0.01 | 30 | −0.17 | 0.38 | E-A | 0.167 |
| F) W/NTX 0.001 | 30 | −0.13 | 0.57 | F-A | 0.094 |
| | | | | C-B | 0.174 |
| | | | | D-B | 0.943 |
| | | | | E-B | 0.462 |
| | | | | F-B | 0.672 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE 75B
Efficacy Results - Means and Standard Deviations for the Categorical
PID Scores (Safety Patients)
Male Patients

| CATEGORICAL PID SCORES | | | | | |
|---|---|---|---|---|---|
| TREATMENT | N | MEAN | SD | SOURCE | P-VALUE |
| 15 MINUTES | | | | | |
| A) Placebo | 22 | 0.23 | 0.69 | TRT | 0.894 |
| B) HC/APAP | 16 | 0.06 | 0.44 | B-A | 0.355 |
| C) W/NTX 1 | 19 | 0.11 | 0.57 | C-A | 0.472 |
| D) W/NTX 0.1 | 15 | 0.13 | 0.52 | D-A | 0.604 |
| E) W/NTX 0.01 | 19 | 0.16 | 0.37 | E-A | 0.682 |
| F) W/NTX 0.001 | 20 | 0.25 | 0.55 | F-A | 0.892 |
| | | | | C-B | 0.816 |
| | | | | D-B | 0.716 |
| | | | | E-B | 0.604 |
| | | | | F-B | 0.303 |
| 30 MINUTES | | | | | |
| A) Placebo | 22 | 0.32 | 0.78 | TRT | 0.159 |
| B) HC/APAP | 16 | 0.50 | 0.52 | B-A | 0.415 |
| C) W/NTX 1 | 19 | 0.42 | 0.90 | C-A | 0.628 |
| D) W/NTX 0.1 | 15 | 0.40 | 0.51 | D-A | 0.718 |
| E) W/NTX 0.01 | 19 | 0.37 | 0.50 | E-A | 0.813 |
| F) W/NTX 0.001 | 20 | 0.85 | 0.67 | F-A | 0.012 |
| | | | | C-B | 0.731 |
| | | | | D-B | 0.681 |
| | | | | E-B | 0.567 |
| | | | | F-B | 0.126 |
| 45 MINUTES | | | | | |
| A) Placebo | 22 | 0.27 | 0.83 | TRT | 0.015 |
| B) HC/APAP | 16 | 0.63 | 0.50 | B-A | 0.133 |
| C) W/NTX 1 | 19 | 0.58 | 0.84 | C-A | 0.170 |
| D) W/NTX 0.1 | 15 | 0.67 | 0.90 | D-A | 0.100 |
| E) W/NTX 0.01 | 19 | 0.53 | 0.51 | E-A | 0.255 |
| F) W/NTX 0.001 | 20 | 1.10 | 0.55 | F-A | <0.001 |
| | | | | C-B | 0.848 |
| | | | | D-B | 0.870 |
| | | | | E-B | 0.682 |
| | | | | F-B | 0.048 |
| 1 HOUR | | | | | |
| A) Placebo | 22 | 0.32 | 1.09 | TRT | 0.030 |
| B) HC/APAP | 16 | 0.69 | 0.48 | B-A | 0.192 |
| C) W/NTX 1 | 19 | 0.37 | 0.90 | C-A | 0.852 |
| D) W/NTX 0.1 | 15 | 0.80 | 0.94 | D-A | 0.095 |
| E) W/NTX 0.01 | 19 | 0.76 | 0.71 | E-A | 0.100 |
| F) W/NTX 0.001 | 20 | 1.15 | 0.81 | F-A | 0.002 |
| | | | | C-B | 0.274 |
| | | | | D-B | 0.715 |
| | | | | E-B | 0.795 |
| | | | | F-B | 0.110 |
| 1.5 HOURS | | | | | |
| A) Placebo | 22 | 0.14 | 0.89 | TRT | 0.019 |
| B) HC/APAP | 16 | 0.56 | 0.63 | B-A | 0.124 |
| C) W/NTX 1 | 19 | 0.37 | 0.90 | C-A | 0.378 |
| D) W/NTX 0.1 | 15 | 0.73 | 0.96 | D-A | 0.036 |
| E) W/NTX 0.01 | 19 | 0.53 | 0.70 | E-A | 0.140 |
| F) W/NTX 0.001 | 20 | 1.05 | 0.89 | F-A | <0.001 |
| | | | | C-B | 0.496 |
| | | | | D-B | 0.571 |
| | | | | E-B | 0.899 |
| | | | | F-B | 0.085 |
| 2 HOURS | | | | | |
| A) Placebo | 22 | −0.09 | 0.92 | TRT | 0.096 |
| B) HC/APAP | 16 | 0.31 | 0.70 | B-A | 0.157 |
| C) W/NTX 1 | 19 | 0.26 | 0.93 | C-A | 0.193 |
| D) W/NTX 0.1 | 15 | 0.47 | 0.99 | D-A | 0.056 |
| E) W/NTX 0.01 | 19 | 0.21 | 0.54 | E-A | 0.267 |
| F) W/NTX 0.001 | 20 | 0.70 | 0.98 | F-A | 0.004 |
| | | | | C-B | 0.866 |
| | | | | D-B | 0.620 |
| | | | | E-B | 0.728 |
| | | | | F-B | 0.183 |
| 3 HOURS | | | | | |
| A) Placebo | 22 | −0.18 | 0.91 | TRT | 0.079 |
| B) HC/APAP | 16 | 0.19 | 0.66 | B-A | 0.151 |
| C) W/NTX 1 | 19 | 0.05 | 0.78 | C-A | 0.338 |
| D) W/NTX 0.1 | 15 | 0.16 | 0.75 | D-A | 0.187 |
| E) W/NTX 0.01 | 19 | 0.00 | 0.33 | E-A | 0.457 |
| F) W/NTX 0.001 | 20 | 0.55 | 1.00 | F-A | 0.003 |
| | | | | C-B | 0.610 |
| | | | | D-B | 0.933 |
| | | | | E-B | 0.479 |
| | | | | F-B | 0.167 |
| 4 HOURS | | | | | |
| A) Placebo | 22 | −0.23 | 0.87 | TRT | 0.029 |
| B) HC/APAP | 16 | 0.13 | 0.50 | B-A | 0.132 |
| C) W/NTX 1 | 19 | −0.11 | 0.57 | C-A | 0.582 |
| D) W/NTX 0.1 | 15 | 0.07 | 0.70 | D-A | 0.216 |
| E) W/NTX 0.01 | 19 | −0.05 | 0.23 | E-A | 0.431 |
| F) W/NTX 0.001 | 20 | 0.50 | 1.00 | F-A | 0.001 |
| | | | | C-B | 0.338 |
| | | | | D-B | 0.819 |
| | | | | E-B | 0.460 |
| | | | | F-B | 0.116 |
| 5 HOURS | | | | | |
| A) Placebo | 22 | −0.27 | 0.70 | TRT | 0.043 |
| B) HC/APAP | 16 | 0.06 | 0.44 | B-A | 0.095 |
| C) W/NTX 1 | 19 | −0.21 | 0.42 | C-A | 0.744 |
| D) W/NTX 0.1 | 15 | 0.07 | 0.70 | D-A | 0.097 |
| E) W/NTX 0.01 | 19 | −0.05 | 0.23 | E-A | 0.249 |
| F) W/NTX 0.001 | 20 | 0.30 | 0.86 | F-A | 0.003 |
| | | | | C-B | 0.187 |
| | | | | D-B | 0.985 |
| | | | | E-B | 0.577 |
| | | | | F-B | 0.245 |
| 6 HOURS | | | | | |
| A) Placebo | 22 | −0.23 | 0.87 | TRT | 0.386 |
| B) HC/APAP | 16 | 0.00 | 0.37 | B-A | 0.245 |
| C) W/NTX 1 | 19 | −0.21 | 0.42 | C-A | 0.928 |
| D) W/NTX 0.1 | 15 | 0.07 | 0.70 | D-A | 0.141 |
| E) W/NTX 0.01 | 19 | −0.05 | 0.23 | E-A | 0.348 |
| F) W/NTX 0.001 | 20 | 0.10 | 0.64 | F-A | 0.076 |
| | | | | C-B | 0.296 |
| | | | | D-B | 0.754 |
| | | | | E-B | 0.794 |
| | | | | F-B | 0.615 |
| 7 HOURS | | | | | |
| A) Placebo | 22 | −0.23 | 0.87 | TRT | 0.386 |
| B) HC/APAP | 16 | 0.00 | 0.37 | B-A | 0.245 |
| C) W/NTX 1 | 19 | −0.21 | 0.42 | C-A | 0.928 |
| D) W/NTX 0.1 | 15 | 0.07 | 0.70 | D-A | 0.141 |
| E) W/NTX 0.01 | 19 | −0.05 | 0.23 | E-A | 0.348 |
| F) W/NTX 0.001 | 20 | 0.10 | 0.64 | F-A | 0.076 |
| | | | | C-B | 0.296 |
| | | | | D-B | 0.754 |
| | | | | E-B | 0.794 |
| | | | | F-B | 0.615 |
| 8 HOURS | | | | | |
| A) Placebo | 22 | −0.27 | 0.70 | TRT | 0.198 |
| B) HC/APAP | 16 | 0.00 | 0.37 | B-A | 0.131 |
| C) W/NTX 1 | 19 | −0.21 | 0.42 | C-A | 0.716 |
| D) W/NTX 0.1 | 15 | 0.07 | 0.70 | D-A | 0.066 |
| E) W/NTX 0.01 | 19 | −0.05 | 0.23 | E-A | 0.200 |
| F) W/NTX 0.001 | 20 | 0.10 | 0.64 | F-A | 0.029 |
| | | | | C-B | 0.258 |
| | | | | D-B | 0.734 |
| | | | | E-B | 0.777 |
| | | | | F-B | 0.586 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED
LSD TEST (MEANING, PAIRWISE P-VALUES ARE ONLY OBSERVED IF THE OVERALL TREATMENT EFFECT IS SIGNIFICANT).

TABLE 75C
Efficacy Results - Means and Standard Deviations for
PEAK PID (Safety Patients) Female Patients

| PEAK PIP SCORES | | | | | |
|---|---|---|---|---|---|
| TREATMENT | | | | | |
| PEAK PID | N | MEAN | SD | SOURCE | P-VALUE |
| A) Placebo | 28 | 0.57 | 0.79 | TRT | 0.130 |
| B) HC/APAP | 34 | 0.94 | 0.85 | B-A | 0.077 |
| C) W/NTX 1 | 31 | 1.09 | 0.83 | C-A | 0.015 |
| D) W/NTX 0.1 | 35 | 0.97 | 0.62 | D-A | 0.054 |
| E) W/NTX 0.01 | 31 | 0.77 | 0.92 | E-A | 0.341 |
| F) W/NTX 0.001 | 30 | 1.07 | 0.87 | F-A | 0.022 |
| | | | | C-B | 0.450 |
| | | | | D-B | 0.878 |
| | | | | E-B | 0.410 |
| | | | | F-B | 0.539 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNTFICANT).

TABLE 75D
Efficacy Results - Means and Standard Deviations for
PEAK PID (Safety Patients) Male Patients

| PEAK PID SCORES | | | | | |
|---|---|---|---|---|---|
| TREATMENT | | | | | |
| PEAK PID | N | MEAN | SD | SOURCE | P-VALUE |
| A) Placebo | 22 | 0.86 | 1.08 | TRT | 0.120 |
| B) HC/APAP | 16 | 0.88 | 0.50 | B-A | 0.964 |
| C) W/NTX 1 | 19 | 0.74 | 0.73 | C-A | 0.600 |
| D) W/NTX 0.1 | 15 | 0.87 | 0.83 | D-A | 0.991 |
| E) W/NTX 0.01 | 19 | 0.89 | 0.66 | E-A | 0.898 |
| F) W/NTX 0.001 | 20 | 1.40 | 0.60 | F-A | 0.026 |
| | | | | C-B | 0.598 |
| | | | | D-B | 0.976 |
| | | | | E-B | 0.940 |
| | | | | F-B | 0.045 |

MEANS GIVEN ARE LEAST SQUARE MEANS.
THE CATEGORICAL SCALE FOR PAIN INTENSITY WAS: 0 = NONE, 1 = MILD, 2 = MODERATE, AND 3 = SEVERE.
OVERALL TREATMENT P-VALUE FROM A ONE-WAY ANOVA, WHILE PAIRWISE RESULTS ARE FROM FISHER'S PROTECTED LSD TEST (MEANING, PAIRWISE P-VALUES ARE ONLY OBSERVED IF THE OVERALL TREATMENT EFFECT IS SIGNIFICANT).

Tables 76A for females and 76B for males present the summary and analysis of global assessments. In females, the placebo group had the highest percentage of “poor” assessments. The 0.1 mg NTX and the 0.001 mg NTX combination groups had the highest percentage of “good” to “excellent” ratings. In males, the placebo group had the highest percentage of “poor” assessments. The 0.001 mg NTX combination group had the highest percentage of “good” to “excellent” ratings.

TABLE 76A
Efficacy Results - Patient Global Assessments (Safety Patients)
Female Patients

| | NUMBER | | | | | | | |
|---|---|---|---|---|---|---|---|---|
| | OF | | | | VERY | | | |
| TREATMENT | PATIENTS | POOR | FAIR | GOOD | GOOD | EXCELLENT | SOURCE | P-VALUE |
| A) Placebo | 28 | 15 (54%) | 7 (25%) | 5 (18%) | 1 (4%) | 0 (0%) | TRT | 0.035 |
| B) HC/APAP | 34 | 10 (29%) | 7 (21%) | 9 (26%) | 4 (12%) | 4 (12%) | B-A | 0.120 |
| C) W/NTX 1 | 31 | 7 (23%) | 7 (23%) | 8 (26%) | 5 (16%) | 4 (13%) | C-A | 0.041 |
| D) W/NTX 0.1 | 35 | 9 (26%) | 6 (17%) | 12 (34%) | 6 (17%) | 2 (6%) | D-A | 0.056 |
| E) W/NTX 0.01 | 31 | 7 (23%) | 12 (39%) | 5 (16%) | 7 (23%) | 0 (0%) | E-A | 0.038 |
| F) W/NTX 0.001 | 30 | 7 (23%) | 6 (20%) | 8 (27%) | 8 (27%) | 1 (3%) | F-A | 0.042 |
| TOTAL | 189 | 55 (29%) | 45 (24%) | 47 (25%) | 31 (16%) | 11 (6%) | C-B | 0.968 |
| | | | | | | | D-B | 0.811 |
| | | | | | | | E-B | 0.109 |
| | | | | | | | F-B | 0.477 |

OVERALL P-VALUE (AND ANY PAIRWISE RESULTS) FROM THE COCHRAN-MANTEL-HAENSZEL TEST FOR ROW MEAN SCORES.

TABLE 76B
Efficacy Results - Patient Global Assessments (Safety Patients)
Male Patients

| | NUMBER | | | | | | | |
|---|---|---|---|---|---|---|---|---|
| | OF | | | | VERY | | | |
| TREATMENT | PATIENTS | POOR | FAIR | GOOD | GOOD | EXCELLENT | SOURCE | P-VALUE |
| A) Placebo | 22 | 11 (50%) | 4 (18%) | 3 (14%) | 4 (18%) | 0 (0%) | TRT | 0.147 |
| B) HC/APAP | 16 | 3 (19%) | 8 (50%) | 3 (19%) | 2 (13%) | 0 (0%) | B-A | 0.132 |
| C) W/NTX 1 | 19 | 5 (26%) | 5 (26%) | 7 (37%) | 2 (11%) | 0 (0%) | C-A | 0.229 |
| D) W/NTX 0.1 | 15 | 6 (40%) | 2 (13%) | 3 (20%) | 3 (20%) | 1 (7%) | D-A | 0.741 |
| E) W/NTX 0.01 | 19 | 6 (32%) | 7 (37%) | 3 (16%) | 3 (16%) | 0 (0%) | E-A | 0.538 |
| F) W/NTX 0.001 | 20 | 2 (10%) | 5 (25%) | 6 (30%) | 5 (25%) | 2 (10%) | F-A | 0.057 |
| TOTAL | 111 | 33 (30%) | 31 (28%) | 25 (23%) | 19 (17%) | 3 (3%) | C-B | 0.479 |
| | | | | | | | D-B | 0.232 |
| | | | | | | | E-B | 0.804 |
| | | | | | | | F-B | 0.324 |

OVERALL P-VALUE (AND ANY PAIRWISE RESULTS) FROM THE COCHRAN-MANTEL-HAENSZEL TEST FOR ROW MEAN SCORES.

The majority of adverse side effects (adverse events) reported were categorized as digestive (nausea or vomiting) or nervous system (dizziness or somnolence) as further shown above in Tables 77A for females and 77B for males.

In females, the placebo group had the lowest incidence of nausea and vomiting. The 0.01 mg NTX combination group had the lowest incidence of dizziness. The placebo, 1.0 mg NTX and the 0.01 mg NTX combination groups had the lowest incidence of sedation.

In males, the HC/APAP alone group had the lowest incidence of nausea. The HC/APAP alone and the 1.0 mg NTX combination groups had the lowest incidence of vomiting. The placebo, HC/APAP alone, and 0.01 mg NTX combination groups had the lowest incidence of dizziness. The 1.0 mg NTX, 0.1 mg NTX and 0.01 mg NTX combination groups had the lowest incidence of sedation.

FIGS. 40A for females and 40B for males represent a summary of exemplary adverse side effects according to methods and compositions of the invention.

TABLE 77A
Summary Of Adverse Events By Body System And Preferred Term
Safety Patients, Female Patients

| BODY SYSTEM | | | |
|---|---|---|---|
| ADVERSE | | TOTAL | NO. OF |
| EVENTS (COSTART | | NO. OF | SUBJECTS |
| ENGLISH) | TREATMENT | SUBJECTS | W/EVENT |
| ALL BODY | A) PLACEBO | 28 | 11 (39%) |
| SYSTEMS | B) HC/APAP | 34 | 13 (38%) |
| | C) W/NTX 1 | 31 | 18 (58%) |
| | D) W/NTX 0.1 mg | 35 | 14 (40%) |
| | E) W/NTX 0.01 mg | 31 | 15 (48%) |
| | F) W/NTX 0.001 | 30 | 15 (50%) |
| | TOTAL | 189 | 86 (46%) |
| GASTRO- | A) PLACEBO | 28 | 8 (29%) |
| INTESTINAL | B) HC/APAP | 34 | 13 (38%) |
| DISORDERS | C) W/NTX 1 | 31 | 15 (48%) |
| | D) W/NTX 0.1 mg | 35 | 12 (34%) |
| | F) W/NTX 0.01 mg | 31 | 13 (42%) |
| | F) W/NTX 0.001 | 30 | 15 (50%) |
| | TOTAL | 189 | 76 (40%) |
| Abdominal | A) PLACEBO | 28 | 0 (0%) |
| Distension | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Abdominal | A) PLACEBO | 28 | 0 (0%) |
| Pain Nos | B) HC/APAP | 34 | 0 (0%) |
| | C) W/NTX 1 | 31 | 0 (0%) |
| | D) W/NTX 0.1 mg | 35 | 1 (3%) |
| | E) W/NTX 0.01 mg | 31 | 0 (0%) |
| | F) W/NTX 0.001 | 30 | 0 (0%) |
| | TOTAL | 189 | 1 (1%) |
| Abdominal Pain | A) PLACEBO | 28 | 0 (0%) |
| Upper | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Constipation | A) PLACEBO | 28 | 0 (0%) |
| | B) HC/APAP | 34 | 0 (0%) |
| | C) W/NTX 1 | 31 | 0 (0%) |
| | D) W/NTX 0.1 mg | 35 | 1 (3%) |
| | E) W/NTX 0.01 mg | 31 | 0 (0%) |
| | F) W/NTX 0.001 | 30 | 1 (3%) |
| | TOTAL | 189 | 2 (1%) |
| Diarrhea Nos | A) PLACEBO | 28 | 0 (0%) |
| | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 1 (3%) |
| | TOTAL | 189 | 2 (1%) |
| Dyspepsia | A) PLACEBO | 28 | 1 (4%) |
| | B) HC/APAP | 34 | 0 (0%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Flatulence | A) PLACEBO | 28 | 0 (0%) |
| | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 1 (3%) |
| | F) W/NTX 0.001 | 30 | 0 (0%) |
| | TOTAL | 189 | 2 (1%) |
| Nausea | A) PLACEBO | 28 | 7 (25%) |
| | B) HC/APAP | 34 | 13 (38%) |
| | C) W/NTX 1 | 31 | 15 (48%) |
| | D) W/NTX 0.1 mg | 35 | 12 (34%) |
| | E) W/NTX 0.01 mg | 31 | 10 (32%) |
| | F) W/NTX 0.001 | 30 | 14 (47%) |
| | TOTAL | 189 | 71 (38%) |
| Vomiting Nos | A) PLACEBO | 28 | 2 (7%) |
| | B) HC/APAP | 34 | 6 (18%) |
| | C) W/NTX 1 | 31 | 4 (13%) |
| | D) W/NTX 0.1 mg | 35 | 5 (14%) |
| | E) W/NTX 0.01 mg | 31 | 7 (23%) |
| | F) W/NTX 0.001 | 30 | 3 (10%) |
| | TOTAL | 189 | 27 (14%) |
| GENERAL | A) PLACEBO | 28 | 0 (0%) |
| DISORDERS AND | B) HC/APAP | 34 | 1 (3%) |
| ADMIN. SITE | C) W/NTX 1 | 31 | 1 (3%) |
| CONDITIONS | D) W/NTX 0.1 mg | 35 | 0 (0%) |
| | E) W/NTX 0.01 mg | 31 | 1 (3%) |
| | F) W/NTX 0.001 | 30 | 0 (0%) |
| | TOTAL | 189 | 3 (2%) |
| Application Site | A) PLACEBO | 28 | 0 (0%) |
| Bleeding | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Pyrexia | A) PLACEBO | 28 | 0 (0%) |
| | B) HC/APAP | 34 | 0 (0%) |
| | C) W/NTX 1 | 31 | 0 (0%) |
| | D) W/NTX 0.1 mg | 35 | 0 (0%) |
| | E) W/NTX 0.01 mg | 31 | 1 (3%) |
| | F) W/NTX 0.001 | 30 | 0 (0%) |
| | TOTAL | 189 | 1 (1%) |
| Rigors | A) PLACEBO | 28 | 0 (0%) |
| | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| NERVOUS | A) PLACEBO | 28 | 4 (14%) |
| SYSTEM | B) HC/APAP | 34 | 5 (15%) |
| DISORDERS | C) W/NTX 1 | 31 | 4 (13%) |
| | D) W/NTX 0.1 mg | 35 | 7 (20%) |
| | E) W/NTX 0.01 mg | 31 | 4 (13%) |
| | F) W/NTX 0.001 | 30 | 6 (20%) |
| | TOTAL | 189 | 30 (16%) |
| Dizziness exc. | A) PLACEBO | 28 | 2 (7%) |
| Vertigo | B) HC/APAP | 34 | 2 (6%) |
| | C) W/NTX 1 | 31 | 4 (13%) |
| | D) W/NTX 0.1 mg | 35 | 5 (14%) |
| | E) W/NTX 0.01 mg | 31 | 0 (0%) |
| | F) W/NTX 0.001 | 30 | 4 (13%) |
| | TOTAL | 189 | 17 (9%) |
| Headache Nos | A) PLACEBO | 28 | 1 (4%) |
| | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 2 (6%) |
| | F) W/NTX 0.001 | 30 | 0 (0%) |
| | TOTAL | 189 | 4 (2%) |
| Migraine Nos | A) PLACEBO | 28 | 0 (0%) |
| | B) HC/APAP | 34 | 0 (0%) |
| | C) W/NTX 1 | 31 | 0 (0%) |
| | D) W/NTX 0.1 mg | 35 | 0 (0%) |
| | E) W/NTX 0.01 mg | 31 | 1 (3%) |
| | F) W/NTX 0.001 | 30 | 0 (0%) |
| | TOTAL | 189 | 1 (1%) |
| Sedation | A) PLACEBO | 28 | 0 (0%) |
| | B) HC/APAP | 34 | 1 (3%) |
| | C) W/NTX 1 | 31 | 0 (0%) |
| | D) W/NTX 0.1 mg | 35 | 2 (6%) |
| | E) W/NTX 0.01 mg | 31 | 0 (0%) |
| | F) W/NTX 0.001 | 30 | 1 (3%) |
| | TOTAL | 189 | 4 (2%) |
| Syncope | A) PLACEBO | 28 | 1 (4%) |
| | B) HC/APAP | 34 | 1 (3%) |
| | C) W/NTX 1 | 31 | 1 (3%) |
| | D) W/NTX 0.1 mg | 35 | 1 (3%) |
| | E) W/NTX 0.01 mg | 31 | 1 (3%) |
| | F) W/NTX 0.001 | 30 | 1 (3%) |
| | TOTAL | 189 | 6 (3%) |
| Tremor Nec | A) PLACEBO | 28 | 0 (0%) |
| | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| PSYCHIATRIC | A) PLACEBO | 28 | 0 (0%) |
| DISORDERS | B) HC/APAP | 34 | 1 (3%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 2 (1%) |
| Anxiety Nec | A) PLACEBO | 28 | 0 (0%) |
| | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Crying | A) PLACEBO | 28 | 0 (0%) |
| | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Nervousness | A) PLACEBO | 28 | 0 (0%) |
| | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| RESPIRATORY, | A) PLACEBO | 28 | 0 (0%) |
| THORACIC | B) HC/APAP | 34 | 1 (3%) |
| AND | C) W/NTX 1 | 31 | 0 (0%) |
| MEDIASTINAL | D) W/NTX 0.1 mg | 35 | 0 (0%) |
| DISORDERS | E) W/NTX 0.01 mg | 31 | 0 (0%) |
| | F) W/NTX 0.001 | 30 | 0 (0%) |
| | TOTAL | 189 | 1 (1%) |
| Respiratory | A) PLACEBO | 28 | 0 (0%) |
| Disorder Nos | B) HC/APAP | 34 | 1 (3%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| SKIN AND | A) PLACEBO | 28 | 1 (4%) |
| SUBCUTANEOUS | B) HC/APAP | 34 | 1 (3%) |
| TISSUE | C) W/NTX 1 | 31 | 3 (10%) |
| DISORDERS | D) W/NTX 0.1 mg | 35 | 2 (6%) |
| | E) W/NTX 0.01 mg | 31 | 3 (10%) |
| | F) W/NTX 0.001 | 30 | 1 (3%) |
| | TOTAL | 189 | 11 (6%) |
| Face Oedma | A) PLACEBO | 28 | 0 (0%) |
| | B) HC/APAP | 34 | 0 (0%) |
| | C) W/NTX 1 | 31 | 0 (0%) |
| | D) W/NTX 0.1 mg | 35 | 0 (0%) |
| | E) W/NTX 0.01 mg | 31 | 1 (3%) |
| | F) W/NTX 0.001 | 30 | 0 (0%) |
| | TOTAL | 189 | 1 (1%) |
| Pruritus Nos | A) PLACEBO | 28 | 1 (4%) |
| | B) HC/APAP | 34 | 0 (0%) |
| | C) W/NTX 1 | 31 | 0 (0%) |
| | D) W/NTX 0.1 mg | 35 | 1 (3%) |
| | E) W/NTX 0.01 mg | 31 | 2 (6%) |
| | F) W/NTX 0.001 | 30 | 0 (0%) |
| | TOTAL | 189 | 4 (2%) |
| Sweating Increased | A) PLACEBO | 28 | 0 (0%) |
| | B) HC/APAP | 34 | 1 (3%) |
| | C) W/NTX 1 | 31 | 3 (10%) |
| | D) W/NTX 0.1 mg | 35 | 0 (0%) |
| | E) W/NTX 0.01 mg | 31 | 0 (0%) |
| | F) W/NTX 0.001 | 30 | 1 (3%) |
| | TOTAL | 189 | 5 (3%) |
| Urticaria Nos | A) PLACEBO | 28 | 0 (0%) |
| | B) HC/APAP | 34 | 0 (0%) |
| | C) W/NTX 1 | 31 | 0 (0%) |
| | D) W/NTX 0.1 mg | 35 | 1 (3%) |
| | E) W/NTX 0.01 mg | 31 | 0 (0%) |
| | F) W/NTX 0.001 | 30 | 0 (0%) |
| | TOTAL | 189 | 1 (1%) |
| Vascular Disorders | A) PLACEBO | 28 | 1 (4%) |
| | B) HC/APAP | 34 | 0 (0%) |
| | C) W/NTX 1 | 31 | 2 (6%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 3 (2%) |
| Flushing | A) PLACEBO | 28 | 1 (4%) |
| | B) HC/APAP | 34 | 0 (0%) |
| | C) W/NTX 1 | 31 | 0 (0%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Hot Flushes Nos | A) PLACEBO | 28 | 0 (0%) |
| | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Hypertension Nos | A) PLACEBO | 28 | 0 (0%) |
| | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |
| Pallor | A) PLACEBO | 28 | 0 (0%) |
| | B) HC/APAP | 34 | 0 (0%) |
| | C) W/NTX 1 | 31 | 1 (3%) |
| | D) W/NTX 0.1 mg | 35 | 0 (0%) |
| | E) W/NTX 0.01 mg | 31 | 0 (0%) |
| | F) W/NTX 0.001 | 30 | 0 (0%) |
| | TOTAL | 189 | 1 (1%) |

NOTE:
AT EACH LEVEL OF SUMMATION (BODY SYSTEM AND PREFERRED TERMS), PATIENTS REPORTING MORE THAN ONE EVENT ARE COUNTED ONLY ONCE. PERCENTAGES OF PATIENTS FOR EACH TREATMENT GROUP ARE ALSO GIVEN.

TABLE 77B
Summary Of Adverse Events By Body System And Preferred Term
Safety Patients, Male Patients

| BODY SYSTEM | | | NO. OF | |
|---|---|---|---|---|
| ADVERSE EVENTS | | TOTAL NO. OF | SUBJECTS | |
| (COSTART ENGLISH) | TREATMENT | SUBJECTS | W/EVENT | |
| ALL BODY SYSTEMS | A) PLACEBO | 22 | 3 | (14%) |
| | B) HC/APAP | 16 | 2 | (13%) |
| | C) W/NTX 1 | 19 | 5 | (26%) |
| | D) W/NTX 0.1 mg | 15 | 7 | (47%) |
| | E) W/NTX 0.01 mg | 19 | 6 | (32%) |
| | F) W/NTX 0.001 | 20 | 5 | (25%) |
| | TOTAL | 111 | 28 | (25%) |
| EAR AND LABRYRINTH | A) PLACEBO | 22 | 0 | (0%) |
| DISORDERS | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| Tinnitus | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| EYE DISORDERS | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| Vision Blurred | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| GASTROINTESTINAL | A) PLACEBO | 22 | 2 | (9%) |
| DISORDERS | B) HC/APAP | 16 | 1 | (6%) |
| | C) W/NTX 1 | 19 | 2 | (11%) |
| | D) W/NTX 0.1 mg | 15 | 4 | (27%) |
| | E) W/NTX 0.01 mg | 19 | 4 | (21%) |
| | F) W/NTX 0.001 | 20 | 3 | (15%) |
| | TOTAL | 111 | 16 | (14%) |
| Abdominal Pain Upper | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 1 | (5%) |
| | F) W/NTX 0.001 | 20 | 0 | (0%) |
| | TOTAL | 111 | 1 | (1%) |
| Nausea | A) PLACEBO | 22 | 2 | (9%) |
| | B) HC/APAP | 16 | 1 | (6%) |
| | C) W/NTX 1 | 19 | 2 | (11%) |
| | D) W/NTX 0.1 mg | 15 | 3 | (20%) |
| | E) W/NTX 0.01 mg | 19 | 2 | (11%) |
| | F) W/NTX 0.001 | 20 | 3 | (15%) |
| | TOTAL | 111 | 13 | (12%) |
| Sore Throat Nos. | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 1 | (5%) |
| | F) W/NTX 0.001 | 20 | 0 | (0%) |
| | TOTAL | 111 | 1 | (1%) |
| Vomiting Nos | A) PLACEBO | 22 | 1 | (5%) |
| | B) HC/APAP | 16 | 0 | (0%) |
| | C) W/NTX 1 | 19 | 0 | (0%) |
| | D) W/NTX 0.1 mg | 15 | 2 | (13%) |
| | E) W/NTX 0.01 mg | 19 | 1 | (5%) |
| | F) W/NTX 0.001 | 20 | 1 | (5%) |
| | TOTAL | 111 | 5 | (5%) |
| GENERAL DISORDERS AND | A) PLACEBO | 22 | 0 | (0%) |
| ADMIN. SITE CONDITIONS | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 0 | (0%) |
| | F) W/NTX 0.001 | 20 | 1 | (5%) |
| | TOTAL | 111 | 1 | (1%) |
| Fatigue | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 0 | (0%) |
| | F) W/NTX 0.001 | 20 | 1 | (5%) |
| | TOTAL | 111 | 1 | (1%) |
| INJURY AND POISONING | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| Abrasion Nos | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| INVESTIGATIONS | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| Blood Pressure Increased | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| MUSCULOSKELETAL | A) PLACEBO | 22 | 0 | (0%) |
| CONNECT TISSUE AND | B) HC/APAP | 16 | 0 | (0%) |
| BONE DISORDERS | C) W/NTX 1 | 19 | 0 | (0%) |
| | D) W/NTX 0.1 mg | 15 | 1 | (7%) |
| | E) W/NTX 0.01 mg | 19 | 0 | (0%) |
| | F) W/NTX 0.001 | 20 | 0 | (0%) |
| | TOTAL | 111 | 1 | (1%) |
| Neck Pain | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 1 | (7%) |
| | E) W/NTX 0.01 mg | 19 | 0 | (0%) |
| | F) W/NTX 0.001 | 20 | 0 | (0%) |
| | TOTAL | 111 | 1 | (1%) |
| NERVOUS SYSTEM | A) PLACEBO | 22 | 2 | (9%) |
| DISORDERS | B) HC/APAP | 16 | 1 | (6%) |
| | C) W/NTX 1 | 19 | 4 | (21%) |
| | D) W/NTX 0.1 mg | 15 | 4 | (27%) |
| | E) W/NTX 0.01 mg | 19 | 0 | (0%) |
| | F) W/NTX 0.001 | 20 | 4 | (20%) |
| | TOTAL | 111 | 15 | (14%) |
| Dizziness exc. Vertigo | A) PLACEBO | 22 | 0 | (0%) |
| | B) HC/APAP | 16 | 0 | (0%) |
| | C) W/NTX 1 | 19 | 3 | (16%) |
| | D) W/NTX 0.1 mg | 15 | 1 | (7%) |
| | E) W/NTX 0.01 mg | 19 | 0 | (0%) |
| | F) W/NTX 0.001 | 20 | 1 | (5%) |
| | TOTAL | 111 | 5 | (5%) |
| Headache Nos | A) PLACEBO | 22 | 1 | (5%) |
| | B) HC/APAP | 16 | 0 | (0%) |
| | C) W/NTX 1 | 19 | 1 | (5%) |
| | D) W/NTX 0.1 mg | 15 | 1 | (7%) |
| | E) W/NTX 0.01 mg | 19 | 0 | (0%) |
| | F) W/NTX 0.001 | 20 | 1 | (5%) |
| | TOTAL | 111 | 4 | (4%) |
| Sedation | A) PLACEBO | 22 | 1 | (5%) |
| | B) HC/APAP | 16 | 1 | (6%) |
| | C) W/NTX 1 | 19 | 0 | (0%) |
| | D) W/NTX 0.1 mg | 15 | 0 | (0%) |
| | E) W/NTX 0.01 mg | 19 | 0 | (0%) |
| | F) W/NTX 0.001 | 20 | 2 | (10%) |
| | TOTAL | 111 | 4 | (4%) |
| Syncope | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 1 | (7%) |
| | E) W/NTX 0.01 mg | 19 | 0 | (0%) |
| | F) W/NTX 0.001 | 20 | 0 | (0%) |
| | TOTAL | 111 | 2 | (2%) |
| Tremor Nec | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 1 | (7%) |
| | E) W/NTX 0.01 mg | 19 | 0 | (0%) |
| | F) W/NTX 0.001 | 20 | 0 | (0%) |
| | TOTAL | 111 | 1 | (1%) |
| PSYCHIATRIC | A) PLACEBO | 22 | 0 | (0%) |
| DISORDERS | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| Nervousness | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| RENAL AND URINARY | A) PLACEBO | 22 | 0 | (0%) |
| DISORDERS | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 1 | (5%) |
| | F) W/NTX 0.001 | 20 | 0 | (0%) |
| | TOTAL | 111 | 1 | (1%) |
| Difficulty in Micturition | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 1 | (5%) |
| | F) W/NTX 0.001 | 20 | 0 | (0%) |
| | TOTAL | 111 | 1 | (1%) |
| SKIN AND | A) PLACEBO | 22 | 1 | (5%) |
| SUBCUTANEOUS TISSUE | B) HC/APAP | 16 | 0 | (0%) |
| DISORDERS | C) W/NTX 1 | 19 | 1 | (5%) |
| | D) W/NTX 0.1 mg | 15 | 2 | (13%) |
| | E) W/NTX 0.01 mg | 19 | 1 | (5%) |
| | F) W/NTX 0.001 | 20 | 1 | (5%) |
| | TOTAL | 111 | 6 | (5%) |
| Pruritus Nos | A) PLACEBO | 22 | 1 | (5%) |
| | B) HC/APAP | 16 | 0 | (0%) |
| | C) W/NTX 1 | 19 | 0 | (0%) |
| | D) W/NTX 0.1 mg | 15 | 1 | (7%) |
| | E) W/NTX 0.01 mg | 19 | 0 | (0%) |
| | F) W/NTX 0.001 | 20 | 0 | (0%) |
| | TOTAL | 111 | 2 | (2%) |
| Sweating Increased | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 1 | (7%) |
| | E) W/NTX 0.01 mg | 19 | 1 | (5%) |
| | F) W/NTX 0.001 | 20 | 1 | (5%) |
| | TOTAL | 111 | 4 | (4%) |
| VASCULAR DISORDERS | A) PLACEBO | 22 | 0 | (0%) |
| | B) HC/APAP | 16 | 0 | (0%) |
| | C) W/NTX 1 | 19 | 2 | (11%) |
| | D) W/NTX 0.1 mg | 15 | 0 | (0%) |
| | E) W/NTX 0.01 mg | 19 | 1 | (5%) |
| | F) W/NTX 0.001 | 20 | 1 | (5%) |
| | TOTAL | 111 | 4 | (4%) |
| Hot Flushes Nos | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 0 | (0%) |
| | TOTAL | 111 | 1 | (1%) |
| Hypertension Nos | A) PLACEBO | 22 | 0 | (0%) |
| | B) HC/APAP | 16 | 0 | (0%) |
| | C) W/NTX 1 | 19 | 0 | (0%) |
| | D) W/NTX 0.1 mg | 15 | 0 | (0%) |
| | E) W/NTX 0.01 mg | 19 | 1 | (5%) |
| | F) W/NTX 0.001 | 20 | 0 | (0%) |
| | TOTAL | 111 | 1 | (1%) |
| Pallor | A) PLACEBO | 22 | 0 | (0%) |
| | B) HC/APAP | 16 | 0 | (0%) |
| | C) W/NTX 1 | 19 | 1 | (5%) |
| | D) W/NTX 0.1 mg | 15 | 0 | (0%) |
| | E) W/NTX 0.01 mg | 19 | 0 | (0%) |
| | F) W/NTX 0.001 | 20 | 1 | (5%) |
| | TOTAL | 111 | 2 | (2%) |

NOTE:
AT EACH LEVEL OF SUMMATION (BODY SYSTEM AND PREFERRED TERMS), PATIENTS REPORTING MORE THAN ONE EVENT ARE COUNTED ONLY ONCE. PERCENTAGES OF PATIENTS FOR EACH TREATMENT GROUP ARE ALSO GIVEN.

### EXAMPLE 7

An additional dose ranging clinical study with morphine sulfate (MS or morphine) alone or in combination with low doses of naltrexone hydrochloride (NTX or naltrexone) was designed substantially the same as that described in Example 1, with the following differences: (1) seven treatment groups (not 5) with three different doses of MS (30 mg, 60 mg and 90 mg) alone or in combination with 0.1 mg NTX versus placebo alone, in subjects with moderate to severe pain in a postsurgical dental pain clinical study; (2) each group was 30 patients (not 40) for a total of 210 males only (not 200 females and males); (3) subjects had three or four third molars, including at least one mandibular partial or complete bony impaction (not 2 or more impacted third molars); (4) time to onset of analgesia (not time to onset of meaningful and perceptible pain relief or time to onset of meaningful pain relief) was measured; (5) the primary efficacy variable was SPID measured through 4 hours (not TOTPAR and SPID measured through 8 hours); (6) the secondary efficacy variables included: 4, 6 and 8 hour Total Pain Relief Scores (TOTPAR-4, TOTPAR-6, and TOTPAR-8); MAXPAR scores; pain relief (PR) scores; 6 and 8 hour Sum of Pain Intensity Difference Scores (SPID-6 and SPID-8); categorical PID scores (pain intensity differences on the categorical scale); PEAKPID scores; VAS-PID scores (pain intensity differences on the visual analog scale); PEAK-VAS-SPID scores; VAS-SPID-4, -6 and -8 scores; (7) additional exclusion criteria were patients with known history of severe hepatic or renal impairment; and (8) for adverse events, body systems and preferred terms were from the MedDRA (not the COSTART) dictionary.

A total of 210 male subjects were randomized; among them all 210 subjects were deemed evaluable (Table 78).

TABLE 78
Analysis Populations
Treatments

| | | | | | E | F | G | |
|---|---|---|---|---|---|---|---|---|
| | | B | C | D | MS (30 mg) | MS (60 mg) | MS (90 mg) | |
| | A | MS | MS | MS | with NTX | with NTX | with NTX | |
| Population | Placebo | (30 mg) | (60 mg) | (90 mg) | (0.1 mg) | (0.1 mg) | (0.1 mg) | Total |
| Safety | 31 | 30 | 30 | 30 | 31 | 30 | 28 | 210 |
| Primary Efficacy | 31 | 30 | 30 | 30 | 31 | 30 | 28 | 210 |
| Per Protocol | 31 | 30 | 30 | 30 | 31 | 30 | 28 | 210 |

The demographic and baseline characteristics were summarized by treatment groups for all 210 randomized patients which were all evaluable (Table 79). Demographic characteristics included age, race/ethnicity, sex, weight, height, medical history, teeth extracted (impacted and non-impacted), baseline pain intensity, and baseline visual analog scale.

Subjects ranged in age from 16 to 49 years; 62.9% were Caucasian and all were male. No adjustments in the analyses were made to take into account differences among treatment groups. These differences had little or no influence on pain assessments at baseline. The baseline pain intensity scores and visual analog scale scores were generally comparable across treatment groups (Tables 80A and 80B).

TABLE 79
Baseline Demographic Characteristics
Primary Efficacy Population

| | | Treatments | | | | | | | | |
|---|---|---|---|---|---|---|---|---|---|---|
| | | | | | | E | F | G | | |
| | | | | | | MS (30 mg) | MS (60 mg) | MS (90 mg) | | |
| | | A | B | C | D | with NTX | with NTX | with NTX | | |
| | | Placebo | MS (30 mg) | MS (60 mg) | MS (90 mg) | (0.1 mg) | (0.1 mg) | (0.1 mg) | Total | P-Value |
| Age (yrs) | N | 31 | 30 | 30 | 30 | 31 | 30 | 28 | 210 | 0.363 |
| | Mean | 23.3 | 25.0 | 22.5 | 24.6 | 22.3 | 24.6 | 23.3 | 23.6 | |
| | SD | 5.49 | 5.48 | 5.14 | 6.06 | 4.56 | 6.69 | 5.52 | 5.60 | |
| | Median | 21.0 | 24.0 | 21.0 | 23.0 | 22.0 | 24.0 | 22.0 | 22.0 | |
| | Range | 17-43 | 16-34 | 16-37 | 16-40 | 16-36 | 17-49 | 16-38 | 16-49 | |
| Height (cm) | N | 31 | 30 | 30 | 30 | 31 | 30 | 28 | 210 | 0.899 |
| | Mean | 177.8 | 176.8 | 177.0 | 175.3 | 176.1 | 175.5 | 176.3 | 176.4 | |
| | SD | 7.63 | 10.18 | 7.02 | 8.07 | 9.26 | 6.82 | 6.49 | 7.97 | |
| | Median | 177.8 | 175.3 | 177.8 | 176.0 | 176.5 | 174.2 | 175.3 | 176.2 | |
| | Range | 162.6-190.5 | 152.4-208.3 | 162.6-195.6 | 150.7-191.8 | 154.9-195.6 | 165.1-185.4 | 167.6-193.0 | 150.7-208.3 | |
| Weight (kg) | N | 31 | 30 | 30 | 30 | 31 | 30 | 28 | 210 | 0.852 |
| | Mean | 80.3 | 81.9 | 83.3 | 81.7 | 82.3 | 82.5 | 77.6 | 81.4 | |
| | SD | 15.38 | 15.05 | 21.75 | 13.62 | 12.44 | 15.09 | 12.57 | 15.30 | |
| | Median | 77.3 | 80.0 | 75.8 | 78.8 | 78.0 | 81.4 | 76.4 | 78.0 | |
| | Range | 56.7-123.6 | 55.3-113.6 | 52.6-140.5 | 65.0-124.5 | 57.3-109.3 | 61.4-116.8 | 61.4-105.0 | 52.6-140.5 | |
| Race/ | Asian | 2 (6.5%) | 1 (3.3%) | 1 (3.3%) | 1 (3.3%) | 0 (0.0%) | 1 (3.3%) | 0 (0.0%) | 6 (2.9%) | 0.946 |
| Ethnic | Black | 1 (3.2%) | 2 (6.7%) | 1 (3.3%) | 1 (3.3%) | 2 (6.5%) | 0 (0.0%) | 0 (0.0%) | 7 (3.3%) | |
| Origin | Caucasian | 18 (58.1%) | 17 (56.7%) | 21 (70.0%) | 20 (66.7%) | 17 (54.8%) | 20 (66.7%) | 19 (67.9%) | 132 (62.9%) | |
| (N, %) | Hispanic | 10 (32.3%) | 9 (30.0%) | 7 (23.3%) | 7 (23.3%) | 12 (38.7%) | 8 (26.7%) | 9 (32.1%) | 62 (29.5%) | |
| | Other | 0 (0.0%) | 1 (3.3%) | 0 (0.0%) | 1 (3.3%) | 0 (0.0%) | 1 (3.3%) | 0 (0.0%) | 3 (1.4%) | |
| | Total | 31 | 30 | 30 | 30 | 31 | 30 | 28 | 210 | |

NOTE:
P-VALUES ARE FROM ONE-WAY ANALYSIS OF VARIANCE FOR AGE, HEIGHT, AND WEIGHT AND FROM CHI-SQUARE TEST FOR RACE/ETHNIC ORIGIN.

TABLE 80A
Baseline Pain Intensity Scores (Categorical)
Primary Efficacy Population

| PAIN INTENSITY | | | | P-VALUE | |
|---|---|---|---|---|---|
| TREATMENT | N | MODERATE | SEVERE | SOURCE | P-VALUE |
| A) Placebo | 31 | 18 (58.1%) | 13 (41.9%) | TREATMENT | 0.999 |
| B) MS 30 mg | 30 | 18 (60.0%) | 12 (40.0%) | | |
| C) MS 60 mg | 30 | 18 (60.0%) | 12 (40.0%) | | |
| D) MS 90 mg | 30 | 18 (60.0%) | 12 (40.0%) | | |
| E) MS 30 mg/NTX 0.1 mg | 31 | 18 (58.1%) | 13 (41.9%) | | |
| F) MS 60 mg/NTX 0.1 mg | 30 | 16 (53.3%) | 14 (46.7%) | | |
| G) MS 90 mg/NTX 0.1 mg | 28 | 16 (57.1%) | 12 (42.9%) | | |

NOTE:
P-VALUES ARE FROM ONE-WAY ANALYSIS OF VARIANCE FOR AGE, HEIGHT, AND WEIGHT AND FROM CHI-SQUARE TEST FOR RACE/ETHNIC ORIGIN.

TABLE 80B
Baseline Pain Intensity Scores (VAS)
Primary Efficacy Population

| BASELINE VAS SCORE | | | | | | | P-VALUE | |
|---|---|---|---|---|---|---|---|---|
| TREATMENT | N | MEAN | SD | MIN | MEDIAN | MAX | SOURCE | P-VALUE |
| A) Placebo | 31 | 74.5 | 12.20 | 53 | 74.0 | 99 | TREATMENT | 0.407 |
| B) MS 30 mg | 30 | 71.3 | 14.17 | 51 | 68.0 | 97 | MS90 − MS60/ | 0.031* |
| C) MS 60 mg | 30 | 72.6 | 12.13 | 55 | 72.0 | 99 | NTX.1 | |
| D) MS 90 mg | 30 | 69.6 | 12.85 | 50 | 68.0 | 97 | | |
| E) MS 30 mg/NTX 0.1 mg | 31 | 71.5 | 9.88 | 55 | 70.0 | 93 | | |
| F) MS 60 mg/NTX 0.1 mg | 30 | 76.4 | 12.31 | 55 | 76.5 | 100 | | |
| G) MS 90 mg/NTX 0.1 mg | 28 | 72.0 | 11.08 | 52 | 71.5 | 98 | | |

[1] FOR AGE, HEIGHT, WEIGHT, AND TIME BETWEEN END OF SURGERY AND STUDY MEDICATION, P-VALUES ARE FROM TWO-WAY ANALYSIS OF VARIANCE WITH TREATMENT AND SITE AS FACTORS; FOR GENDER, RACE/ETHNIC ORIGIN, AND NUMBER OF THIRD MOLARS EXTRACTED, P-VALUES ARE FROM COCHRAN-MANTEL-HAENZEL TEST ADJUSTING FOR SITE.
[2] BLACK, ASIAN, HISPANIC, AND OTHER ARE COMBINED INTO ONE CATEGORY TO DERIVE P-VALUE.
[3] 4 OR MORE THIRD MOLARS EXTRACTED AS ONE CATEGORY TO DERIVE P-VALUE.

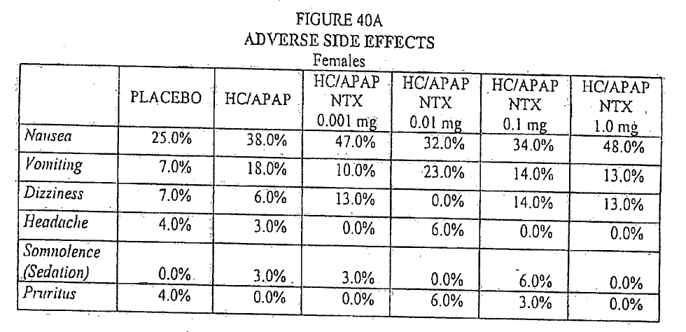

The sum of pain relief (total pain relief or TOTPAR) scores (4 hour, 6 hour, 8 hour) are summarized in Table 81 and the mean 4 hour scores are shown in FIG. 41. The placebo treatment group had the lowest mean TOTPAR scores. All 6 of the active treatment groups with 30 mg, 60 mg or 90 mg MS alone or in combination with 0.1 mg NTX exhibited mean TOTPAR scores that were numerically higher than placebo. The mean TOTPAR score for the 90 mg MS/0.1 mg NTX combination treatment was the highest among all treatment groups.

The mean TOTPAR scores for the 30 mg, 60 mg and 90 mg MS alone treatment groups were comparable. In contrast, the mean TOTPAR scores for the 30 mg MS/0; 1 mg NTX, 60 mg MS/0.1 mg NTX and 90 mg/MS 0.1 mg NTX combination treatment groups demonstrated a dose response as shown in Table 81 and FIG. 41.

TABLE 81
Sum of Pain Relief Scores (TOTPAR)
Primary Efficacy Population

| TOTAL PAIN RELIEF SCORE | | | | | | | | |
|---|---|---|---|---|---|---|---|---|
| TREATMENT | N | MEAN | SD | MIN | MEDIAN | MAX | SOURCE | P-VALUE |

TOTAL PAIN RELIEF SCORE (0-4 HOURS)

| | | | | | | | | |
|---|---|---|---|---|---|---|---|---|
| A. Placebo | 31 | 2.4 | 3.47 | 0.0 | 0.4 | 11.7 | TRT | <0.001*** |
| B. MS 30 mg | 30 | 4.1 | 3.20 | 0.0 | 4.5 | 11.2 | A-B | 0.050 |
| C. MS 60 mg | 30 | 4.7 | 3.59 | 0.0 | 4.9 | 11.9 | A-C | 0.011* |
| D. MS 90 mg | 30 | 4.5 | 3.71 | 0.0 | 4.2 | 12.6 | A-D | 0.020* |
| E. MS 30 mg/NTX 0.1 mg | 31 | 3.8 | 3.54 | 0.0 | 3.8 | 9.9 | A-E | 0.106 |
| F. MS 60 mg/NTX 0.1 mg | 30 | 4.4 | 3.73 | 0.0 | 4.3 | 13.3 | A-F | 0.025* |
| G. MS 90 mg/NTX 0.1 mg | 28 | 6.8 | 3.10 | 0.0 | 7.4 | 11.6 | A-G | <0.001*** |
| | | | | | | | B-C | 0.555 |
| | | | | | | | B-D | 0.705 |
| | | | | | | | B-E | 0.720 |
| | | | | | | | B-F | 0.775 |
| | | | | | | | B-G | 0.004** |
| | | | | | | | C-D | 0.833 |
| | | | | | | | C-E | 0.341 |
| | | | | | | | C-F | 0.761 |
| | | | | | | | C-G | 0.021* |
| | | | | | | | D-E | 0.459 |
| | | | | | | | D-F | 0.926 |
| | | | | | | | D-G | 0.012* |
| | | | | | | | E-F | 0.518 |
| | | | | | | | E-G | 0.001** |
| | | | | | | | F-G | 0.009** |

TOTAL PAIN RELIEF SCORE (0-6 HOURS)

| | | | | | | | | |
|---|---|---|---|---|---|---|---|---|
| A. Placebo | 31 | 4.1 | 5.95 | 0.0 | 0.4 | 19.7 | TRT | <0.001*** |
| B. MS 30 mg | 30 | 7.4 | 5.79 | 0.0 | 8.9 | 17.7 | A-B | 0.027* |
| C. MS 60 mg | 30 | 7.8 | 5.88 | 0.0 | 8.4 | 17.9 | A-C | 0.016* |
| D. MS 90 mg | 30 | 7.6 | 6.17 | 0.0 | 8.1 | 20.1 | A-D | 0.021* |
| E. MS 30 mg/NTX 0.1 mg | 31 | 6.7 | 6.33 | 0.0 | 6.5 | 17.9 | A-E | 0.084 |
| F. MS 60 mg/NTX 0.1 mg | 30 | 7.6 | 6.09 | 0.0 | 6.9 | 21.3 | A-F | 0.020* |
| G. MS 90 mg/NTX 0.1 mg | 28 | 11.5 | 5.32 | 0.0 | 12.9 | 19.6 | A-G | <0.001*** |
| | | | | | | | B-C | 0.830 |
| | | | | | | | B-D | 0.918 |
| | | | | | | | B-E | 0.618 |
| | | | | | | | B-F | 0.901 |
| | | | | | | | B-G | 0.010* |
| | | | | | | | C-D | 0.910 |
| | | | | | | | C-E | 0.474 |
| | | | | | | | C-F | 0.927 |
| | | | | | | | C-G | 0.019* |
| | | | | | | | D-E | 0.547 |
| | | | | | | | D-F | 0.983 |
| | | | | | | | D-G | 0.014* |
| | | | | | | | E-F | 0.532 |
| | | | | | | | E-G | 0.002** |
| | | | | | | | F-G | 0.015* |

TOTAL PAIN RELIEF SCORE (0-8 HOURS)

| | | | | | | | | |
|---|---|---|---|---|---|---|---|---|
| A. Placebo | 31 | 5.8 | 8.56 | 0.0 | 0.4 | 27.7 | TRT | 0.001** |
| B. MS 30 mg | 30 | 10.8 | 8.46 | 0.0 | 13.4 | 25.7 | A-B | 0.024* |
| C. MS 60 mg | 30 | 11.1 | 8.47 | 0.0 | 11.4 | 24.4 | A-C | 0.016* |
| D. MS 90 mg | 30 | 11.1 | 8.84 | 0.0 | 13.4 | 26.1 | A-D | 0.017* |
| E. MS 30 mg/NTX 0.1 mg | 31 | 9.6 | 9.21 | 0.0 | 8.8 | 25.9 | A-E | 0.083 |
| F. MS 60 mg/NTX 0.1 mg | 30 | 11.0 | 8.71 | 0.0 | 11.4 | 29.3 | A-F | 0.018* |
| G. MS 90 mg/NTX 0.1 mg | 28 | 16.4 | 7.73 | 0.0 | 18.4 | 27.6 | A-G | <0.001*** |
| | | | | | | | B-C | 0.887 |
| | | | | | | | B-D | 0.890 |
| | | | | | | | B-E | 0.586 |
| | | | | | | | B-F | 0.919 |
| | | | | | | | B-G | 0.013* |
| | | | | | | | C-D | 0.997 |
| | | | | | | | C-E | 0.491 |
| | | | | | | | C-F | 0.967 |
| | | | | | | | C-G | 0.019* |
| | | | | | | | D-E | 0.494 |
| | | | | | | | D-F | 0.970 |
| | | | | | | | D-G | 0.019 |
| | | | | | | | E-F | 0.518 |
| | | | | | | | E-G | 0.003** |
| | | | | | | | F-G | 0.018* |

NOTE:
P-VALUES ARE FROM ONE-WAY ANALYSIS OF VARIANCE.

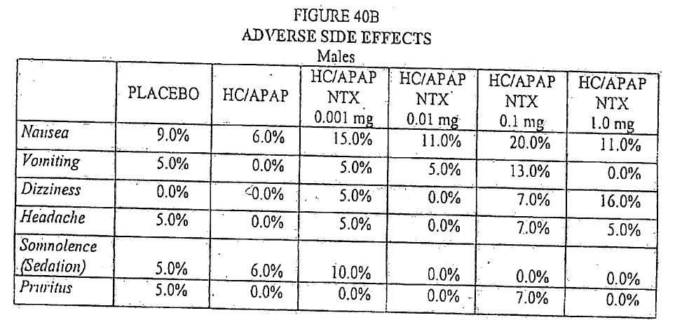

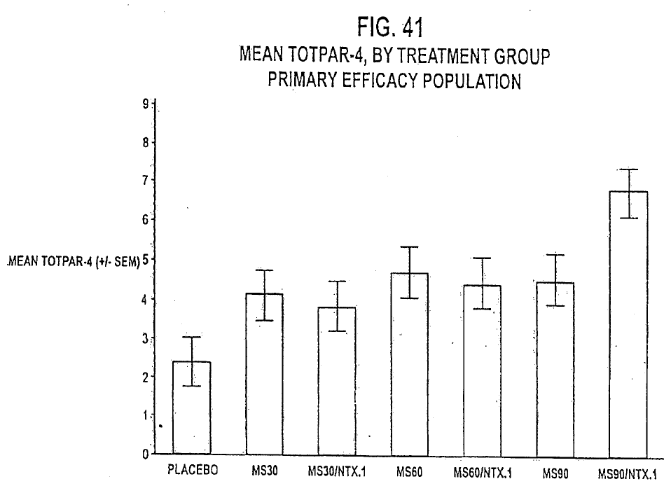

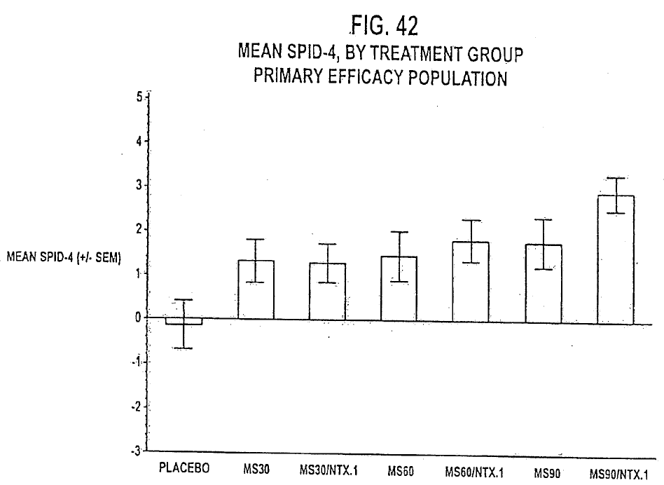

Table 82 summarizes the 4, 6, and 8 hour sum of pain intensity difference (SPID) scores. The mean 4 hour results are also represented in FIG. 42. The placebo treatment group had the lowest mean 4 hour SPID scores. All 6 of the active treatment groups with 30 mg, 60 mg or 90 mg MS alone or in combination with 0.1 mg NTX exhibited improved profiles in mean SPID relative to placebo. The mean 4 hour SPID score for the 90 mg MS/0.1 mg NTX combination treatment was the highest among all treatment groups.

The mean SPID scores for the 30 mg, 60 mg and 90 mg MS alone treatment groups were comparable. In contrast the mean SPID scores for the 30 mg MS/0.1 mg NTX, 60 mg MS/0.1 mg NTX and 9b mg MS/0.1 mg NTX combination treatment groups demonstrated a dose response as shown in Table 82 and FIG. 42.

TABLE 82
Sum of Pain Intensity Difference Scores (SPID)
Primary Efficacy Population

| SUM OF PAIN INTENSITY DIFFERENCE | | | | | | | | |
|---|---|---|---|---|---|---|---|---|
| TREATMENT | N | MEAN | SD | MIN | MEDIAN | MAX | SOURCE | P-VALUE |

SUM OF PAIN INTENSITY DIFFERENCE (0-4 HOURS)

| | | | | | | | | |
|---|---|---|---|---|---|---|---|---|
| A. Placebo | 31 | −0.1 | 3.01 | −3.8 | 0.0 | 8.1 | TRT | 0.004** |
| B. MS 30 mg | 30 | 1.3 | 2.62 | −3.8 | 1.4 | 6.1 | A-B | 0.040* |
| C. MS 60 mg | 30 | 1.5 | 3.09 | −3.8 | 2.0 | 8.4 | A-C | 0.024* |
| D. MS 90 mg | 30 | 1.8 | 3.04 | −3.8 | 2.1 | 9.1 | A-D | 0.007** |
| E. MS 30 mg/NTX 0.1 mg | 31 | 1.3 | 2.38 | −3.8 | 0.0 | 6.7 | A-E | 0.042* |
| F. MS 60 mg/NTX 0.1 mg | 30 | 1.8 | 2.62 | −3.5 | 1.7 | 7.3 | A-F | 0.006** |
| G. MS 90 mg/NTX 0.1 mg | 28 | 2.9 | 2.08 | −0.3 | 3.2 | 7.0 | A-G | <0.001*** |
| | | | | | | | B-C | 0.834 |
| | | | | | | | B-D | 0.508 |
| | | | | | | | B-E | 0.969 |
| | | | | | | | B-F | 0.475 |
| | | | | | | | B-G | 0.026* |
| | | | | | | | C-D | 0.651 |
| | | | | | | | C-E | 0.803 |
| | | | | | | | C-F | 0.613 |
| | | | | | | | C-G | 0.042* |
| | | | | | | | D-E | 0.480 |
| | | | | | | | D-F | 0.958 |
| | | | | | | | D-G | 0.111 |
| | | | | | | | E-F | 0.448 |
| | | | | | | | E-G | 0.022* |
| | | | | | | | F-G | 0.123 |

SUM OF PAIN INTENSITY DIFFERENCE (0-6 HOURS)

| | | | | | | | | |
|---|---|---|---|---|---|---|---|---|
| A. Placebo | 31 | −0.0 | 5.03 | −5.8 | 0.0 | 14.1 | TRT | 0.004** |
| B. MS 30 mg | 30 | 2.6 | 4.50 | −5.8 | 2.5 | 10.1 | A-B | 0.024* |
| C. MS 60 mg | 30 | 2.6 | 4.92 | −5.8 | 5.2 | 12.4 | A-C | 0.024* |
| D. MS 90 mg | 30 | 3.1 | 4.93 | −5.8 | 4.1 | 14.6 | A-D | 0.008** |
| E. MS 30 mg/NTX 0.1 mg | 31 | 2.4 | 4.39 | −5.8 | 0.0 | 12.7 | A-E | 0.033* |
| F. MS 60 mg/NTX 0.1 mg | 30 | 3.2 | 4.35 | −5.5 | 3.1 | 12.8 | A-F | 0.007** |
| G. MS 90 mg/NTX 0.1 mg | 28 | 5.1 | 3.48 | −0.3 | 5.5 | 11.0 | A-G | <0.001*** |
| | | | | | | | B-C | 0.997 |
| | | | | | | | B-D | 0.682 |
| | | | | | | | B-E | 0.876 |
| | | | | | | | B-F | 0.648 |
| | | | | | | | B-G | 0.039* |
| | | | | | | | C-D | 0.679 |
| | | | | | | | C-E | 0.879 |
| | | | | | | | C-F | 0.645 |
| | | | | | | | C-G | 0.038* |
| | | | | | | | D-E | 0.569 |
| | | | | | | | D-F | 0.962 |
| | | | | | | | D-G | 0.095 |
| | | | | | | | E-F | 0.537 |
| | | | | | | | E-G | 0.026* |
| | | | | | | | F-G | 0.105 |

SUM OF PAIN INTENSITY DIFFERENCE (8 HOURS)

| | | | | | | | | |
|---|---|---|---|---|---|---|---|---|
| A. Placebo | 31 | 0.0 | 7.16 | −7.8 | 0.0 | 20.1 | TRT | 0.004** |
| B. MS 30 mg | 30 | 3.9 | 6.40 | −7.8 | 4.5 | 13.6 | A-B | 0.020* |
| C. MS 60 mg | 30 | 3.9 | 6.79 | −7.8 | 7.2 | 16.9 | A-C | 0.021* |
| D. MS 90 mg | 30 | 4.6 | 6.91 | −7.8 | 6.1 | 18.6 | A-D | 0.007** |
| E. MS 30 mg/NTX 0.1 mg | 31 | 3.6 | 6.46 | −7.8 | 0.0 | 18.7 | A-E | 0.033* |
| F. MS 60 mg/NTX 0.1 mg | 30 | 4.6 | 6.33 | −7.5 | 3.6 | 18.8 | A-F | 0.006** |
| G. MS 90 mg/NTX 0.1 mg | 28 | 7.5 | 5.01 | −0.3 | 7.7 | 15.0 | A-G | <0.001*** |
| | | | | | | | B-C | 0.990 |
| | | | | | | | B-D | 0.684 |
| | | | | | | | B-E | 0.839 |
| | | | | | | | B-F | 0.682 |
| | | | | | | | B-G | 0.040* |
| | | | | | | | C-D | 0.675 |
| | | | | | | | C-E | 0.849 |
| | | | | | | | C-F | 0.673 |
| | | | | | | | C-G | 0.039* |
| | | | | | | | D-E | 0.540 |
| | | | | | | | D-F | 0.997 |
| | | | | | | | D-G | 0.097 |
| | | | | | | | E-F | 0.538 |
| | | | | | | | E-G | 0.023* |
| | | | | | | | F-G | 0.097 |

NOTE:
P-VALUES ARE FROM ONE-WAY ANALYSIS OF VARIANCE.

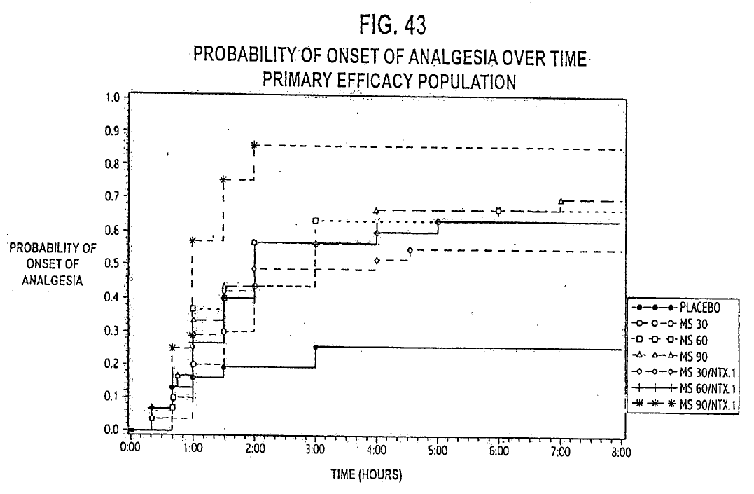

FIG. 43 is a visual presentation of the summary and analysis of time to onset of analgesia presented in Table 83. The median time to onset of analgesia was shortest in the 90 mg MS/0.1 mg NTX combination treatment group.

TABLE 83
Time to Onset of Analgesia
Primary Efficacy Population

| | | MEDIAN | 95% CONFIDENCE | | | |
|---|---|---|---|---|---|---|
| | | TIME | INTERVAL | | | |
| TREATMENT | N | (hh:mm) | (hh:mm) | SOURCE | LOG-RANK | WILCOXON |
| A) Placebo | 31 | >8:00 | (>8:00, >8:00) | TRT | <0.001*** | <0.001*** |
| B) MS 30 mg | 30 | 3:00 | (2:00, >8:00) | A-B | 0.009** | 0.023* |
| C) MS 60 mg | 30 | 2:00 | (1:00, >8:00) | A-C | 0.003** | 0.008** |
| D) MS 90 mg | 30 | 2:00 | (1:00, 7:00) | A-D | 0.001** | 0.004** |
| E) MS 30 mg/NTX 0.1 mg | 31 | 4:00 | (1:30, >8:00) | A-E | 0.029* | 0.048* |
| F) MS 60 mg/NTX 0.1 mg | 30 | 3:00 | (1:30, >8:00) | A-F | 0.006** | 0.014* |
| G) MS 90 mg/NTX 0.1 mg | 28 | 1:00 | (1:00, 1:30) | A-G | <0.001*** | <0.001*** |
| | | | | B-C | 0.537 | 0.341 |
| | | | | B-D | 0.407 | 0.289 |
| | | | | B-E | 0.826 | 0.869 |
| | | | | B-F | 0.817 | 0.659 |
| | | | | B-G | 0.002** | <0.001*** |
| | | | | C-D | 0.780 | 0.815 |
| | | | | C-E | 0.468 | 0.550 |
| | | | | C-F | 0.778 | 0.767 |
| | | | | C-G | 0.017* | 0.013* |
| | | | | D-E | 0.306 | 0.401 |
| | | | | D-F | 0.601 | 0.635 |
| | | | | D-G | 0.043* | 0.036* |
| | | | | E-F | 0.621 | 0.720 |
| | | | | E-G | 0.005** | 0.006** |
| | | | | F-G | 0.011* | 0.013* |

Note:
median time and its confidence interval are estimated using kaplan-meier method. Log-rank and wilcoxon tests are used to test the equality of Kaplan-Meier survival functions over different treatment groups.

Table 84 summarizes the results of the time to remedication (see also FIG. 44). The placebo group had the shortest median time to remedication and the 90 mg MS/0.1 mg NTX combination treatment group had the longest median time to remedication.

TABLE 84
Time to Re-Medication
Primary Efficacy Population

| | | MEDIAN | 95% CONFIDENCE | | | |
|---|---|---|---|---|---|---|
| TREATMENT | N | TIME (hh:mm) | INTERVAL (hh:mm) | SOURCE | LOG-RANK | WILCOXON |
| A) Placebo | 31 | 1:38 | (1:35, 2:07) | TRT | <0.001*** | <0.001*** |
| B) MS 30 mg | 30 | 8:33 | (2:31, 9:55) | A-B | 0.003** | <0.001*** |
| C) MS 60 mg | 30 | 7:17 | (2:08, 10:13) | A-C | 0.012* | 0.002** |
| D) MS 90 mg | 30 | 9:09 | (2:09, >24:00) | A-D | <0.001*** | <0.001*** |
| E) MS 30 mg/NTX 0.1 mg | 31 | 2:23 | (1:40, 9:53) | A-E | 0.073 | 0.043* |
| F) MS 60 mg/NTX 0.1 mg | 30 | 5:23 | (2:09, 10:17) | A-F | 0.003** | <0.001*** |
| G) MS 90 mg/NTX 0.1 mg | 28 | 9:50 | (6:06, 12:26) | A-G | <0.001*** | <0.001*** |
| | | | | B-C | 0.699 | 0.723 |
| | | | | B-D | 0.265 | 0.607 |
| | | | | B-E | 0.349 | 0.159 |
| | | | | B-F | 0.828 | 0.830 |
| | | | | B-G | 0.162 | 0.250 |
| | | | | C-D | 0.109 | 0.353 |
| | | | | C-E | 0.598 | 0.334 |
| | | | | C-F | 0.477 | 0.807 |
| | | | | C-G | 0.060 | 0.120 |
| | | | | D-E | 0.037* | 0.067 |
| | | | | D-F | 0.444 | 0.586 |
| | | | | D-G | 0.802 | 0.602 |
| | | | | E-F | 0.202 | 0.209 |
| | | | | E-G | 0.023* | 0.021* |
| | | | | F-G | 0.275 | 0.221 |

NOTE:
MEDIAN TIME AND ITS CONFIDENCE INTERVAL ARE ESTIMATED USING KAPLAN-MEIER METHOD. LOG-RANK AND WILCOXON TESTS ARE USED TO TEST THE EQUALITY OF KAPLAN-MEIER SURVIVAL FUNCTIONS OVER DIFFERENT TREATMENT GROUPS.

The summary and analysis of percent of subjects who took rescue medication up to 4, 8 and 24 hours are presented in Table 85. More than 70% of subjects at 4 hours in the 90 mg MS/0.1 mg NTX combination group and more than 60% of subjects in the same combination group at 8 hours did not require rescue medication.

TABLE 85
Time to Re-Medicated
Primary Efficacy Population

| | | RE-MEDICATED | | | |
|---|---|---|---|---|---|
| TREATMENT | N | YES | NO | SOURCE | P-VALUE |

EFFICACY OBSERVATION PERIOD (0-4 HOURS)

| | | | | | |
|---|---|---|---|---|---|
| A) Placebo | 31 | 24 (77.42%) | 7 (22.58%) | TRT | 0.007** |
| B) MS 30 mg. | 30 | 13 (43.33%) | 17 (56.67%) | A-B | 0.006** |
| C) MS 60 mg | 30 | 12 (40.00%) | 18 (60.00%) | A-C | 0.003** |
| D) MS 90 mg | 30 | 13 (43.33%) | 17 (56.67%) | A-D | 0.006** |
| E) MS 30 mg/NTX 0.1 mg | 31 | 17 (54.84%) | 14 (45.16%) | A-E | 0.060 |
| F) MS 60 mg/NTX 0.1 mg | 30 | 12 (40.00%) | 18 (60.00%) | A-F | 0.003** |
| G) MS 90 mg/NTX 0.1 mg | 28 | 8 (28.57%) | 20 (71.43%) | A-G | <0.001*** |
| | | | | B-C | 0.793 |
| | | | | B-D | 1.000 |
| | | | | B-E | 0.369 |
| | | | | B-F | 0.793 |
| | | | | B-G | 0.242 |
| | | | | C-D | 0.793 |
| | | | | C-E | 0.246 |
| | | | | C-F | 1.000 |
| | | | | C-G | 0.360 |
| | | | | D-E | 0.369 |
| | | | | D-F | 0.793 |
| | | | | D-G | 0.242 |
| | | | | E-F | 0.246 |
| | | | | E-G | 0.041* |
| | | | | F-G | 0.360 |

EFFICACY OBSERVATION PERIOD (0-8 HOURS)

| | | | | | |
|---|---|---|---|---|---|
| A) Placebo | 31 | 25 (80.65%) | 6 (19.35%) | TRT | 0.021* |
| B) MS 30 mg. | 30 | 14 (46.67%) | 16 (53.33%) | A-B | 0.006** |
| C) MS 60 mg | 30 | 15 (50.00%) | 15 (50.00%) | A-C | 0.012* |
| D) MS 90 mg | 30 | 14 (46.67%) | 16 (53.33%) | A-D | 0.006** |
| E) MS 30 mg/NTX 0.1 mg | 31 | 19 (61.29%) | 12 (38.71%) | A-E | 0.093 |
| F) MS 60 mg/NTX 0.1 mg | 30 | 17 (56.67%) | 13 (43.33%) | A-F | 0.043* |
| G) MS 90 mg/NTX 0.1 mg | 28 | 10 (35.71%) | 18 (64.29%) | A-G | <0.001*** |
| | | | | B-C | 0.796 |
| | | | | B-D | 1.000 |
| | | | | B-E | 0.252 |
| | | | | B-F | 0.438 |
| | | | | B-G | 0.397 |
| | | | | C-D | 0.796 |
| | | | | C-E | 0.375 |
| | | | | C-F | 0.605 |
| | | | | C-G | 0.272 |
| | | | | D-E | 0.252 |
| | | | | D-F | 0.438 |
| | | | | D-G | 0.397 |
| | | | | E-F | 0.714 |
| | | | | E-G | 0.050* |
| | | | | F-G | 0.110 |

EFFICACY OBSERVATION PERIOD (0-24 HOURS)

| | | | | | |
|---|---|---|---|---|---|
| A) Placebo | 31 | 29 (93.55%) | 2 (6.45%) | TRT | 0.026* |
| B) MS 30 mg. | 30 | 25 (83.33%) | 5 (16.67%) | A-B | 0.211 |
| C) MS 60 mg | 30 | 27 (90.00%) | 3 (10.00%) | A-C | 0.614 |
| D) MS 90 mg | 30 | 20 (66.67%) | 10 (33.33%) | A-D | 0.008** |
| E) MS 30 mg/NTX 0.1 mg | 31 | 28 (90.32%) | 3 (9.68%) | A-E | 0.641 |
| F) MS 60 mg/NTX 0.1 mg | 30 | 23 (76.67%) | 7 (23.33%) | A-F | 0.063 |
| G) MS 90 mg/NTX 0.1 mg | 28 | 19 (67.86%) | 9 (32.14%) | A-G | 0.011* |
| | | | | B-C | 0.448 |
| | | | | B-D | 0.136 |
| | | | | B-E | 0.419 |
| | | | | B-F | 0.519 |
| | | | | B-G | 0.169 |
| | | | | C-D | 0.028* |
| | | | | C-E | 0.966 |
| | | | | C-F | 0.166 |
| | | | | C-G | 0.038* |
| | | | | D-E | 0.024* |
| | | | | D-F | 0.390 |
| | | | | D-G | 0.923 |
| | | | | E-F | 0.150 |
| | | | | E-G | 0.032* |
| | | | | F-G | 0.453 |

NOTE:
P-VALUES ARE FROM CHI-SQUARE TEST.

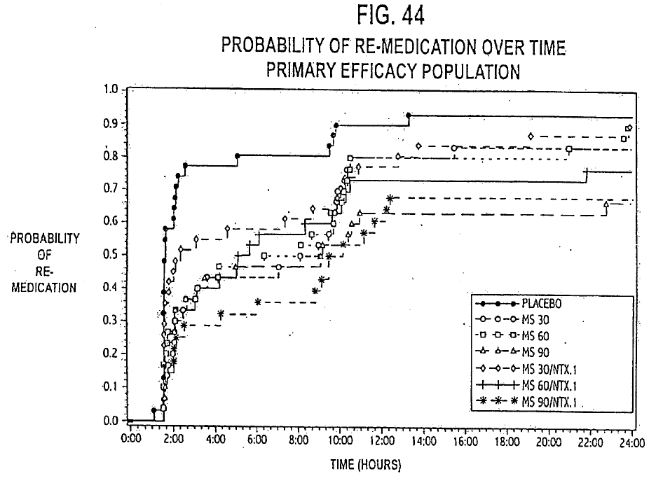

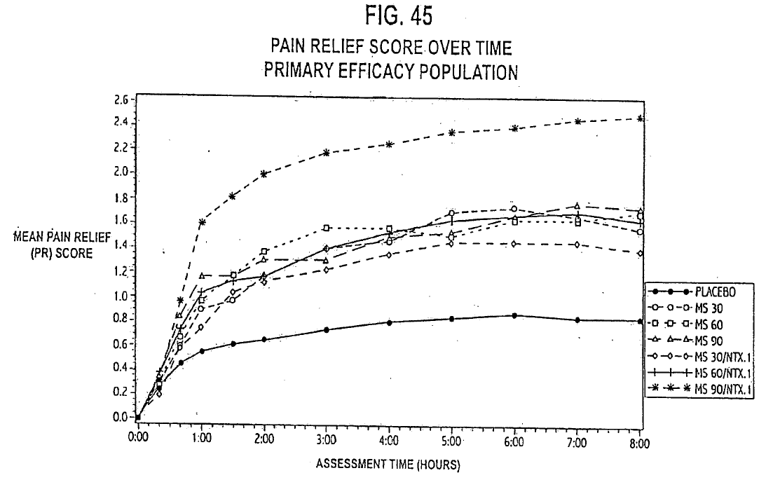

FIG. 45 is a visual presentation of the mean pain relief scores presented in Table 86. The mean pain relief score for the placebo treatment was less than those for the active treatment groups (30 mg, 60 mg, 90 mg MS alone or in combination with 0.1 mg NTX) which improved over time. There was separation between the placebo and the active treatment groups that continued throughout the 8 hour study period. Highest pain relief scores were observed for the 90 mg MS/0.1 mg NTX combination group (FIG. 45).

TABLE 86
Pain Relief (PR) Score
Primary Efficacy Population

| PAIN RELIEF SCORE (PR) | | | | | | | | |
|---|---|---|---|---|---|---|---|---|
| TREATMENT | N | MEAN | SD | MIN | MEDIAN | MAX | SOURCE | P-VALUE |

20 MINUTES

| | | | | | | |
|---|---|---|---|---|---|---|
| A) Placebo | 31 | 0.26 | 0.58 | 0.00 | TRT | 0.881 |
| B) MS 30 mg | 30 | 0.27 | 0.52 | 0.00 | A-B | 0.951 |
| C) MS 60 mg | 30 | 0.30 | 0.47 | 0.00 | A-C | 0.765 |
| D) MS 90 mg | 30 | 0.37 | 0.61 | 0.00 | A-D | 0.440 |
| E) MS 30 mg/NTX 0.1 mg | 31 | 0.19 | 0.48 | 0.00 | A-E | 0.644 |
| F) MS 60 mg/NTX 0.1 mg | 30 | 0.37 | 0.61 | 0.00 | A-F | 0.440 |
| G) MS 90 mg/NTX 0.1 mg | 28 | 0.32 | 0.55 | 0.00 | A-G | 0.658 |
| | | | | | B-C | 0.814 |
| | | | | | B-D | 0.481 |
| | | | | | B-E | 0.603 |
| | | | | | B-F | 0.481 |
| | | | | | B-G | 0.704 |
| | | | | | C-D | 0.638 |
| | | | | | C-E | 0.449 |
| | | | | | C-F | 0.638 |
| | | | | | C-G | 0.882 |
| | | | | | D-E | 0.219 |
| | | | | | D-F | 1.000 |
| | | | | | D-G | 0.754 |
| | | | | | E-F | 0.219 |
| | | | | | E-G | 0.372 |
| | | | | | F-G | 0.754 |

40 MINUTES

| | | | | | | |
|---|---|---|---|---|---|---|
| A) Placebo | 31 | 0.45 | 0.81 | 0.00 | TRT | 0.222 |
| B) MS 30 mg | 30 | 0.60 | 0.67 | 1.00 | A-B | 0.463 |
| C) MS 60 mg | 30 | 0.67 | 0.66 | 1.00 | A-C | 0.288 |
| D) MS 90 mg | 30 | 0.83 | 0.91 | 1.00 | A-D | 0.060 |
| E) MS 30 mg/NTX 0.1 mg | 31 | 0.58 | 0.67 | 0.00 | A-E | 0.520 |
| F) MS 60 mg/NTX 0.1 mg | 30 | 0.73 | 0.83 | 1.00 | A-F | 0.164 |
| G) MS 90 mg/NTX 0.1 mg | 28 | 0.96 | 0.92 | 1.00 | A-G | 0.013* |
| | | | | | B-C | 0.744 |
| | | | | | B-D | 0.253 |
| | | | | | B-E | 0.924 |
| | | | | | B-F | 0.513 |
| | | | | | B-G | 0.080 |
| | | | | | C-D | 0.414 |
| | | | | | C-E | 0.670 |
| | | | | | C-F | 0.744 |
| | | | | | C-G | 0.152 |
| | | | | | D-E | 0.212 |
| | | | | | D-F | 0.624 |
| | | | | | D-G | 0.528 |
| | | | | | E-F | 0.450 |
| | | | | | E-G | 0.063 |
| | | | | | F-G | 0.266 |

60 MINUTES

| | | | | | | |
|---|---|---|---|---|---|---|
| A) Placebo | 31 | 0.55 | 0.89 | 0.00 | TRT | 0.001** |
| B) MS 30 mg | 30 | 0.90 | 0.80 | 1.00 | A-B | 0.143 |
| C) MS 60 mg | 30 | 0.97 | 0.96 | 1.00 | A-C | 0.082 |
| D) MS 90 mg | 30 | 1.17 | 1.09 | 1.00 | A-D | 0.010* |
| E) MS 30 mg/NTX 0.1 mg | 31 | 0.74 | 0.89 | 0.00 | A-E | 0.416 |
| F) MS 60 mg/NTX 0.1 mg | 30 | 1.03 | 1.10 | 1.00 | A-F | 0.044* |
| G) MS 90 mg/NTX 0.1 mg | 28 | 1.61 | 0.74 | 2.00 | A-G | <0.001*** |
| | | | | | B-C | 0.782 |
| | | | | | B-D | 0.270 |
| | | | | | B-E | 0.509 |
| | | | | | B-F | 0.581 |
| | | | | | B-G | 0.004** |
| | | | | | C-D | 0.408 |
| | | | | | C-E | 0.349 |
| | | | | | C-F | 0.782 |
| | | | | | C-G | 0.010** |
| | | | | | D-E | 0.077 |
| | | | | | D-F | 0.581 |
| | | | | | D-G | 0.074 |
| | | | | | E-F | 0.225 |
| | | | | | E-G | <0.001*** |
| | | | | | F-G | 0.020* |

90 MINUTES

| | | | | | | |
|---|---|---|---|---|---|---|
| A) Placebo | 31 | 0.61 | 0.92 | 0.00 | TRT | 0.001** |
| B) MS 30 mg | 30 | 0.97 | 0.81 | 1.00 | A-B | 0.169 |
| C) MS 60 mg | 30 | 1.17 | 0.99 | 1.00 | A-C | 0.032* |
| D) MS 90 mg | 30 | 1.17 | 1.05 | 1.00 | A-D | 0.032* |
| E) MS 30 mg/NTX 0.1 mg | 31 | 1.03 | 1.05 | 1.00 | A-E | 0.100 |
| F) MS 60 mg/NTX 0.1 mg | 30 | 1.13 | 1.22 | 1.00 | A-F | 0.044* |
| G) MS 90 mg/NTX 0.1 mg | 28 | 1.82 | 0.90 | 2.00 | A-G | <0.001*** |
| | | | | | B-C | 0.440 |
| | | | | | B-D | 0.440 |
| | | | | | B-E | 0.798 |
| | | | | | B-F | 0.520 |
| | | | | | B-G | 0.001** |
| | | | | | C-D | 1.000 |
| | | | | | C-E | 0.600 |
| | | | | | C-F | 0.897 |
| | | | | | C-G | 0.014* |
| | | | | | D-E | 0.600 |
| | | | | | D-F | 0.897 |
| | | | | | D-G | 0.014* |
| | | | | | E-F | 0.694 |
| | | | | | E-G | 0.003** |
| | | | | | F-G | 0.010** |

2 HOURS

| | | | | | | |
|---|---|---|---|---|---|---|
| A) Placebo | 31 | 0.65 | 0.98 | 0.00 | TRT | <0.001*** |
| B) MS 30 mg | 30 | 1.17 | 0.95 | 1.00 | A-B | 0.059 |
| C) MS 60 mg | 30 | 1.37 | 1.19 | 1.00 | A-C | 0.009** |
| D) MS 90 mg | 30 | 1.30 | 1.18 | 1.00 | A-D | 0.018* |
| E) MS 30 mg/NTX 0.1 mg | 31 | 1.13 | 1.06 | 1.00 | A-E | 0.078 |
| F) MS 60 mg/NTX 0.1 mg | 30 | 1.17 | 1.12 | 1.00 | A-F | 0.059 |
| G) MS 90 mg/NTX 0.1 mg | 28 | 2.00 | 1.02 | 2.00 | A-G | <0.001*** |
| | | | | | B-C | 0.472 |
| | | | | | B-D | 0.631 |
| | | | | | B-E | 0.891 |
| | | | | | B-F | 1.000 |
| | | | | | B-G | 0.004** |
| | | | | | C-D | 0.810 |
| | | | | | C-E | 0.389 |
| | | | | | C-F | 0.472 |
| | | | | | C-G | 0.026* |
| | | | | | D-E | 0.535 |
| | | | | | D-F | 0.631 |
| | | | | | D-G | 0.014* |
| | | | | | E-F | 0.891 |
| | | | | | E-G | 0.002** |
| | | | | | F-G | 0.004** |

3 HOURS

| | | | | | | |
|---|---|---|---|---|---|---|
| A) Placebo | 31 | 0.74 | 1.12 | 0.00 | TRT | 0.001** |
| B) MS 30 mg | 30 | 1.40 | 1.13 | 2.00 | A-B | 0.031* |
| C) MS 60 mg | 30 | 1.57 | 1.30 | 2.00 | A-C | 0.007** |
| D) MS 90 mg | 30 | 1.30 | 1.15 | 1.00 | A-D | 0.068 |
| E) MS 30 mg/NTX 0.1 mg | 31 | 1.23 | 1.23 | 1.00 | A-E | 0.110 |
| F) MS 60 mg/NTX 0.1 mg | 30 | 1.40 | 1.22 | 1.00 | A-F | 0.031* |
| G) MS 90 mg/NTX 0.1 mg | 28 | 2.18 | 1.12 | 3.00 | A-G | <0.001*** |
| | | | | | B-C | 0.587 |
| | | | | | B-D | 0.744 |
| | | | | | B-E | 0.567 |
| | | | | | B-F | 1.000 |
| | | | | | B-G | 0.013* |
| | | | | | C-D | 0.385 |
| | | | | | C-E | 0.263 |
| | | | | | C-F | 0.587 |
| | | | | | C-G | 0.051 |
| | | | | | D-E | 0.807 |
| | | | | | D-F | 0.744 |
| | | | | | D-G | 0.005** |
| | | | | | E-F | 0.567 |
| | | | | | E-G | 0.002** |
| | | | | | F-G | 0.013* |

4 HOURS

| | | | | | | |
|---|---|---|---|---|---|---|
| A) Placebo | 31 | 0.81 | 1.22 | 0.00 | TRT | 0.005** |
| B) MS 30 mg | 30 | 1.47 | 1.31 | 1.50 | A-B | 0.046* |
| C) MS 60 mg | 30 | 1.57 | 1.30 | 1.50 | A-C | 0.022* |
| D) MS 90 mg | 30 | 1.50 | 1.28 | 2.00 | A-D | 0.036* |
| E) MS 30 mg/NTX 0.1 mg | 31 | 1.35 | 1.40 | 1.00 | A-E | 0.094 |
| F) MS 60 mg/NTX 0.1 mg | 30 | 1.53 | 1.28 | 1.50 | A-F | 0.028* |
| G) MS 90 mg/NTX 0.1 mg | 28 | 2.25 | 1.17 | 3.00 | A-G | <0.001*** |
| | | | | | B-C | 0.763 |
| | | | | | B-D | 0.920 |
| | | | | | B-E | 0.734 |
| | | | | | B-F | 0.841 |
| | | | | | B-G | 0.021* |
| | | | | | C-D | 0.841 |
| | | | | | C-E | 0.520 |
| | | | | | C-F | 0.920 |
| | | | | | C-G | 0.044* |
| | | | | | D-E | 0.660 |
| | | | | | D-F | 0.920 |
| | | | | | D-G | 0.027* |
| | | | | | E-F | 0.588 |
| | | | | | E-G | 0.008** |
| | | | | | F-G | 0.035* |

5 HOURS

| | | | | | | |
|---|---|---|---|---|---|---|
| A) Placebo | 31 | 0.84 | 1.29 | 0.00 | TRT | 0.004** |
| B) MS 30 mg | 30 | 1.70 | 1.39 | 2.00 | A-B | 0.013* |
| C) MS 60 mg | 30 | 1.50 | 1.31 | 1.00 | A-C | 0.055 |
| D) MS 90 mg | 30 | 1.53 | 1.33 | 1.50 | A-D | 0.044* |
| E) MS 30 mg/NTX 0.1 mg | 31 | 1.45 | 1.46 | 1.00 | A-E | 0.073 |
| F) MS 60 mg/NTX 0.1 mg | 30 | 1.63 | 1.35 | 2.00 | A-F | 0.022* |
| G) MS 90 mg/NTX 0.1 mg | 28 | 2.36 | 1.22 | 3.00 | A-G | <0.001*** |
| | | | | | B-C | 0.564 |
| | | | | | B-D | 0.631 |
| | | | | | B-E | 0.470 |
| | | | | | B-F | 0.847 |
| | | | | | B-G | 0.063 |
| | | | | | C-D | 0.923 |
| | | | | | C-E | 0.888 |
| | | | | | C-F | 0.700 |
| | | | | | C-G | 0.016* |
| | | | | | D-E | 0.812 |
| | | | | | D-F | 0.773 |
| | | | | | D-G | 0.020* |
| | | | | | E-F | 0.597 |
| | | | | | E-G | 0.010* |
| | | | | | F-G | 0.041* |

6 HOURS

| | | | | | | |
|---|---|---|---|---|---|---|
| A) Placebo | 31 | 0.87 | 1.36 | 0.00 | TRT | 0.007** |
| B) MS 30 mg | 30 | 1.73 | 1.44 | 2.00 | A-B | 0.016* |
| C) MS 60 mg | 30 | 1.63 | 1.35 | 2.00 | A-C | 0.033* |
| D) MS 90 mg | 30 | 1.67 | 1.42 | 2.00 | A-D | 0.026* |
| E) MS 30 mg/NTX 0.1 mg | 31 | 1.45 | 1.50 | 1.00 | A-E | 0.102 |
| F) MS 60 mg/NTX 0.1 mg | 30 | 1.67 | 1.40 | 2.00 | A-F | 0.026* |
| G) MS 90 mg/NTX 0.1 mg | 28 | 2.39 | 1.23 | 3.00 | A-G | <0.001*** |
| | | | | | B-C | 0.781 |
| | | | | | B-D | 0.853 |
| | | | | | B-E | 0.430 |
| | | | | | B-F | 0.853 |
| | | | | | B-G | 0.072 |
| | | | | | C-D | 0.926 |
| | | | | | C-E | 0.610 |
| | | | | | C-F | 0.926 |
| | | | | | C-G | 0.039* |
| | | | | | D-E | 0.546 |
| | | | | | D-F | 1.000 |
| | | | | | D-G | 0.048* |
| | | | | | E-F | 0.546 |
| | | | | | E-G | 0.010* |
| | | | | | F-G | 0.048* |

7 HOURS

| | | | | | | |
|---|---|---|---|---|---|---|
| A) Placebo | 31 | 0.84 | 1.32 | 0.00 | TRT | 0.003** |
| B) MS 30 mg | 30 | 1.67 | 1.42 | 2.00 | A-B | 0.022* |
| C) MS 60 mg | 30 | 1.63 | 1.38 | 1.50 | A-C | 0.028* |
| D) MS 90 mg | 30 | 1.77 | 1.45 | 2.00 | A-D | 0.011* |
| E) MS 30 mg/NTX 0.1 mg | 31 | 1.45 | 1.52 | 1.00 | A-E | 0.087 |
| F) MS 60 mg/NTX 0.1 mg | 30 | 1.70 | 1.42 | 2.00 | A-F | 0.018* |
| G) MS 90 mg/NTX 0.1 mg | 28 | 2.46 | 1.29 | 3.00 | A-G | <0.001*** |
| | | | | | B-C | 0.927 |
| | | | | | B-D | 0.783 |
| | | | | | B-E | 0.550 |
| | | | | | B-F | 0.927 |
| | | | | | B-G | 0.032* |
| | | | | | C-D | 0.713 |
| | | | | | C-E | 0.614 |
| | | | | | C-F | 0.854 |
| | | | | | C-G | 0.025* |
| | | | | | D-E | 0.382 |
| | | | | | D-F | 0.854 |
| | | | | | D-G | 0.060 |
| | | | | | E-F | 0.490 |
| | | | | | E-G | 0.006** |
| | | | | | F-G | 0.040* |

8 HOURS

| | | | | | | |
|---|---|---|---|---|---|---|
| A) Placebo | 31 | 0.84 | 1.32 | 0.00 | TRT | 0.002** |
| B) MS 30 mg | 30 | 1.57 | 1.38 | 1.50 | A-B | 0.042* |
| C) MS 60 mg | 30 | 1.70 | 1.42 | 2.00 | A-C | 0.017* |
| D) MS 90 mg | 30 | 1.73 | 1.41 | 2.00 | A-D | 0.013* |
| E) MS 30 mg/NTX 0.1 mg | 31 | 1.39 | 1.45 | 1.00 | A-E | 0.122 |
| F) MS 60 mg/NTX 0.1 mg | 30 | 1.63 | 1.40 | 1.50 | A-F | 0.027* |
| G) MS 90 mg/NTX 0.1 mg | 28 | 2.50 | 1.35 | 3.00 | A-G | <0.001*** |
| | | | | | B-C | 0.711 |
| | | | | | B-D | 0.643 |
| | | | | | B-E | 0.615 |
| | | | | | B-F | 0.853 |
| | | | | | B-G | 0.011* |
| | | | | | C-D | 0.926 |
| | | | | | C-E | 0.381 |
| | | | | | C-F | 0.853 |
| | | | | | C-G | 0.030* |
| | | | | | D-E | 0.332 |
| | | | | | D-F | 0.781 |
| | | | | | D-G | 0.037* |
| | | | | | E-F | 0.490 |
| | | | | | E-G | 0.002** |
| | | | | | F-G | 0.019* |

NOTE:
P-VALUES ARE FROM ONE-WAY ANALYSIS OF VARIANCE

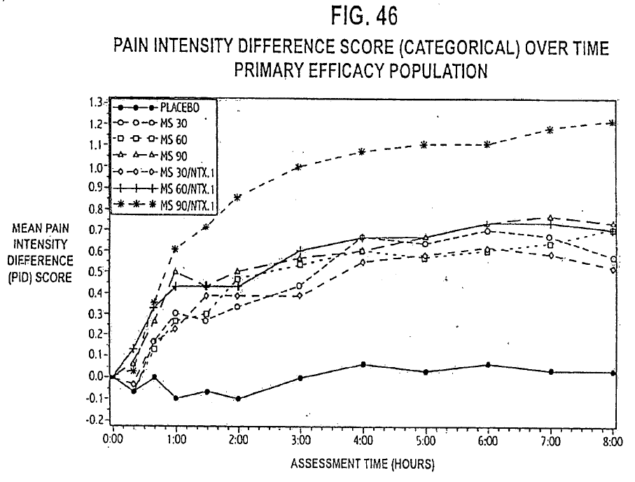

The mean categorical pain intensity difference (PID) scores are presented in Table 87 and FIG. 46. The mean PID scores for the placebo treatment group was generally flat while the mean PID scores generally improved over time for the active treatment groups (30 mg MS, 60 mg MS and 90 mg MS alone or in combination with 0.1 mg NTX). The mean scores for the morphine alone and morphine/naltrexone combination treatment groups were higher than the mean PID scores for the placebo group at each hourly assessment time from 1-8 hours. Highest pain relief as measured by PID scores was observed for the 90 mg MS/0.1 mg NTX combination treatment group.

TABLE 87
Pain Intensity Difference Score (Categorical)
Primary Efficacy Population

| PAIN INTENSITY DIFFERENCE SCORE (Categorical) | | | | | | | | |
|---|---|---|---|---|---|---|---|---|
| TREATMENT | N | MEAN | SD | MIN | MEDIAN | MAX | SOURCE | P-VALUE |
| 20 MINUTES | | | | | | | | |
| A) Placebo | 31 | −0.06 | 0.51 | | 0.00 | | TRT | 0.502 |
| B) MS 30 mg | 30 | −0.07 | 0.45 | | 0.00 | | A-B | 0.985 |
| C) MS 60 mg | 30 | −0.07 | 0.58 | | 0.00 | | A-C | 0.985 |
| D) MS 90 mg | 30 | 0.07 | 0.52 | | 0.00 | | A-D | 0.266 |
| E) MS 30 mg/NTX 0.1 mg | 31 | −0.03 | 0.31 | | 0.00 | | A-E | 0.783 |
| F) MS 60 mg/NTX 0.1 mg | 30 | 0.13 | 0.43 | | 0.00 | | A-F | 0.094 |
| G) MS 90 mg/NTX 0.1 mg | 28 | 0.04 | 0.33 | | 0.00 | | A-G | 0.404 |
| | | | | | | | B-C | 1.000 |
| | | | | | | | B-D | 0.262 |
| | | | | | | | B-E | 0.770 |
| | | | | | | | B-F | 0.093 |
| | | | | | | | B-G | 0.398 |
| | | | | | | | C-D | 0.262 |
| | | | | | | | C-E | 0.770 |
| | | | | | | | C-F | 0.093 |
| | | | | | | | C-G | 0.398 |
| | | | | | | | D-E | 0.402 |
| | | | | | | | D-F | 0.575 |
| | | | | | | | D-G | 0.798 |
| | | | | | | | E-F | 0.161 |
| | | | | | | | E-G | 0.571 |
| | | | | | | | F-G | 0.420 |
| 40 MINUTES | | | | | | | | |
| A) Placebo | 31 | 0.00 | 0.63 | | 0.00 | | TRT | 0.396 |
| B) MS 30 mg | 30 | 0.17 | 0.70 | | 0.00 | | A-B | 0.332 |
| C) MS 60 mg | 30 | 0.13 | 0.68 | | 0.00 | | A-C | 0.437 |
| D) MS 90 mg | 30 | 0.27 | 0.78 | | 0.00 | | A-D | 0.121 |
| E) MS 30 mg/NTX 0.1 mg | 31 | 0.16 | 0.45 | | 0.00 | | A-E | 0.343 |
| F) MS 60 mg/NTX 0.1 mg | 30 | 0.33 | 0.76 | | 0.00 | | A-F | 0.053 |
| G) MS 90 mg/NTX 0.1 mg | 28 | 0.36 | 0.62 | | 0.00 | | A-G | 0.042* |
| | | | | | | | B-C | 0.847 |
| | | | | | | | B-D | 0.563 |
| | | | | | | | B-E | 0.975 |
| | | | | | | | B-F | 0.336 |
| | | | | | | | B-G | 0.280 |
| | | | | | | | C-D | 0.441 |
| | | | | | | | C-E | 0.871 |
| | | | | | | | C-F | 0.248 |
| | | | | | | | C-G | 0.204 |
| | | | | | | | D-E | 0.539 |
| | | | | | | | D-F | 0.700 |
| | | | | | | | D-G | 0.607 |
| | | | | | | | E-F | 0.316 |
| | | | | | | | E-G | 0.263 |
| | | | | | | | F-G | 0.892 |
| 60 MINUTES | | | | | | | | |
| A) Placebo | 31 | −0.10 | 0.75 | | 0.00 | | TRT | 0.012* |
| B) MS 30 mg | 30 | 0.30 | 0.70 | | 0.00 | | A-B | 0.040* |
| C) MS 60 mg | 30 | 0.27 | 0.83 | | 0.00 | | A-C | 0.060 |
| D) MS 90 mg | 30 | 0.50 | 0.90 | | 0.50 | | A-D | 0.002** |
| E) MS 30 mg/NTX 0.1 mg | 31 | 0.23 | 0.62 | | 0.00 | | A-E | 0.091 |
| F) MS 60 mg/NTX 0.1 mg | 30 | 0.43 | 0.82 | | 0.00 | | A-F | 0.006** |
| G) MS 90 mg/NTX 0.1 mg | 28 | 0.61 | 0.57 | | 1.00 | | A-G | <0.001*** |
| | | | | | | | B-C | 0.863 |
| | | | | | | | B-D | 0.302 |
| | | | | | | | B-E | 0.699 |
| | | | | | | | B-F | 0.491 |
| | | | | | | | B-G | 0.120 |
| | | | | | | | C-D | 0.229 |
| | | | | | | | C-E | 0.832 |
| | | | | | | | C-F | 0.390 |
| | | | | | | | C-G | 0.085 |
| | | | | | | | D-E | 0.154 |
| | | | | | | | D-F | 0.731 |
| | | | | | | | D-G | 0.587 |
| | | | | | | | E-F | 0.281 |
| | | | | | | | E-G | 0.052 |
| | | | | | | | F-G | 0.378 |
| 90 MINUTES | | | | | | | | |
| A) Placebo | 31 | −0.06 | 0.85 | | 0.00 | | TRT | 0.012* |
| B) MS 30 mg | 30 | 0.27 | 0.69 | | 0.00 | | A-B | 0.091 |
| C) MS 60 mg | 30 | 0.30 | 0.84 | | 0.00 | | A-C | 0.063 |
| D) MS 90 mg | 30 | 0.43 | 0.86 | | 0.50 | | A-D | 0.011* |
| E) MS 30 mg/NTX 0.1 mg | 31 | 0.39 | 0.67 | | 0.00 | | A-E | 0.021* |
| F) MS 60 mg/NTX 0.1 mg | 30 | 0.43 | 0.77 | | 0.00 | | A-F | 0.011* |
| G) MS 90 mg/NTX 0.1 mg | 28 | 0.71 | 0.60 | | 1.00 | | A-G | <0.001*** |
| | | | | | | | B-C | 0.866 |
| | | | | | | | B-D | 0.398 |
| | | | | | | | B-E | 0.538 |
| | | | | | | | B-F | 0.398 |
| | | | | | | | B-G | 0.026* |
| | | | | | | | C-D | 0.499 |
| | | | | | | | C-E | 0.656 |
| | | | | | | | C-F | 0.499 |
| | | | | | | | C-G | 0.040* |
| | | | | | | | D-E | 0.813 |
| | | | | | | | D-F | 1.000 |
| | | | | | | | D-G | 0.162 |
| | | | | | | | E-F | 0.813 |
| | | | | | | | E-G | 0.101 |
| | | | | | | | F-G | 0.162 |
| 2 HOURS | | | | | | | | |
| A) Placebo | 31 | −0.10 | 0.87 | | 0.00 | | TRT | 0.003** |
| B) MS 30 mg | 30 | 0.33 | 0.76 | | 0.00 | | A-B | 0.042* |
| C) MS 60 mg | 30 | 0.47 | 0.97 | | 0.50 | | A-C | 0.008** |
| D) MS 90 mg | 30 | 0.50 | 0.94 | | 0.50 | | A-D | 0.005** |
| E) MS 30 mg/NTX 0.1 mg | 31 | 0.39 | 0.72 | | 0.00 | | A-E | 0.021* |
| F) MS 60 mg/NTX 0.1 mg | 30 | 0.43 | 0.73 | | 0.00 | | A-F | 0.012* |
| G) MS 90 mg/NTX 0.1 mg | 28 | 0.86 | 0.71 | | 1.00 | | A-G | <0.001*** |
| | | | | | | | B-C | 0.530 |
| | | | | | | | B-D | 0.432 |
| | | | | | | | B-E | 0.798 |
| | | | | | | | B-F | 0.637 |
| | | | | | | | B-G | 0.016* |
| | | | | | | | C-D | 0.875 |
| | | | | | | | C-E | 0.705 |
| | | | | | | | C-F | 0.875 |
| | | | | | | | C-G | 0.071 |
| | | | | | | | D-E | 0.591 |
| | | | | | | | D-F | 0.753 |
| | | | | | | | D-G | 0.099 |
| | | | | | | | E-F | 0.826 |
| | | | | | | | E-G | 0.029* |
| | | | | | | | F-G | 0.051 |
| 3 HOURS | | | | | | | | |
| A) Placebo | 31 | 0.00 | 0.97 | | 0.00 | | TRT | 0.003** |
| B) MS 30 mg | 30 | 0.43 | 0.86 | | 0.00 | | A-B | 0.056 |
| C) MS 60 mg | 30 | 0.53 | 0.97 | | 1.00 | | A-C | 0.019* |
| D) MS 90 mg | 30 | 0.57 | 0.90 | | 1.00 | | A-D | 0.013* |
| E) MS 30 mg/NTX 0.1 mg | 31 | 0.39 | 0.80 | | 0.00 | | A-E | 0.084 |
| F) MS 60 mg/NTX 0.1 mg | 30 | 0.60 | 0.86 | | 0.50 | | A-F | 0.008** |
| G) MS 90 mg/NTX 0.1 mg | 28 | 1.00 | 0.77 | | 1.00 | | A-G | <0.001*** |
| | | | | | | | B-C | 0.660 |
| | | | | | | | B-D | 0.557 |
| | | | | | | | B-E | 0.837 |
| | | | | | | | B-F | 0.463 |
| | | | | | | | B-G | 0.015* |
| | | | | | | | C-D | 0.883 |
| | | | | | | | C-E | 0.517 |
| | | | | | | | C-F | 0.769 |
| | | | | | | | C-G | 0.045* |
| | | | | | | | D-E | 0.426 |
| | | | | | | | D-F | 0.883 |
| | | | | | | | D-G | 0.062 |
| | | | | | | | E-F | 0.345 |
| | | | | | | | E-G | 0.008** |
| | | | | | | | F-G | 0.085 |
| 4 HOURS | | | | | | | | |
| A) Placebo | 31 | 0.06 | 1.03 | | 0.00 | | TRT | 0.012* |
| B) MS 30 mg | 30 | 0.67 | 0.99 | | 1.00 | | A-B | 0.015* |
| C) MS 60 mg | 30 | 0.60 | 1.04 | | 1.00 | | A-C | 0.031* |
| D) MS 90 mg | 30 | 0.60 | 0.97 | | 1.00 | | A-D | 0.031* |
| E) MS 30 mg/NTX 0.1 mg | 31 | 0.55 | 0.99 | | 0.00 | | A-E | 0.049* |
| F) MS 60 mg/NTX 0.1 mg | 30 | 0.67 | 0.88 | | 1.00 | | A-F | 0.015* |
| G) MS 90 mg/NTX 0.1 mg | 28 | 1.07 | 0.77 | | 1.00 | | A-G | <0.001*** |
| | | | | | | | B-C | 0.788 |
| | | | | | | | B-D | 0.788 |
| | | | | | | | B-E | 0.631 |
| | | | | | | | B-F | 1.000 |
| | | | | | | | B-G | 0.110 |
| | | | | | | | C-D | 1.000 |
| | | | | | | | C-E | 0.834 |
| | | | | | | | C-F | 0.788 |
| | | | | | | | C-G | 0.063 |
| | | | | | | | D-E | 0.834 |
| | | | | | | | D-F | 0.788 |
| | | | | | | | D-G | 0.063 |
| | | | | | | | E-F | 0.631 |
| | | | | | | | E-G | 0.038* |
| | | | | | | | F-G | 0.110 |
| 5 HOURS | | | | | | | | |
| A) Placebo | 31 | 0.03 | 1.02 | | 0.00 | | TRT | 0.007** |
| B) MS 30 mg | 30 | 0.63 | 0.96 | | 1.00 | | A-B | 0.018* |
| C) MS 60 mg | 30 | 0.57 | 1.01 | | 1.00 | | A-C | 0.034* |
| D) MS 90 mg | 30 | 0.67 | 1.03 | | 1.00 | | A-D | 0.012* |
| E) MS 30 mg/NTX 0.1 mg | 31 | 0.58 | 1.03 | | 0.00 | | A-E | 0.029* |
| F) MS 60 mg/NTX 0.1 mg | 30 | 0.67 | 0.99 | | 0.00 | | A-F | 0.012* |
| G) MS 90 mg/NTX 0.1 mg | 28 | 1.11 | 0.79 | | 1.00 | | A-G | <0.001*** |
| | | | | | | | B-C | 0.792 |
| | | | | | | | B-D | 0.895 |
| | | | | | | | B-E | 0.834 |
| | | | | | | | B-F | 0.895 |
| | | | | | | | B-G | 0.067 |
| | | | | | | | C-D | 0.693 |
| | | | | | | | C-E | 0.956 |
| | | | | | | | C-F | 0.693 |
| | | | | | | | C-G | 0.037* |
| | | | | | | | D-E | 0.732 |
| | | | | | | | D-F | 1.000 |
| | | | | | | | D-G | 0.089 |
| | | | | | | | E-F | 0.732 |
| | | | | | | | E-G | 0.041* |
| | | | | | | | F-G | 0.089 |
| 6 HOURS | | | | | | | | |
| A) Placebo | 31 | 0.06 | 1.09 | | 0.00 | | TRT | 0.014* |
| B) MS 30 mg | 30 | 0.70 | 1.02 | | 1.00 | | A-B | 0.016* |
| C) MS 60 mg | 30 | 0.60 | 1.00 | | 1.00 | | A-C | 0.042* |
| D) MS 90 mg | 30 | 0.73 | 1.05 | | 1.00 | | A-D | 0.011* |
| E) MS 30 mg/NTX 0.1 mg | 31 | 0.61 | 1.09 | | 0.00 | | A-E | 0.035* |
| F) MS 60 mg/NTX 0.1 mg | 30 | 0.73 | 1.05 | | 0.50 | | A-F | 0.011* |
| G) MS 90 mg/NTX 0.1 mg | 28 | 1.11 | 0.79 | | 1.00 | | A-G | <0.001*** |
| | | | | | | | B-C | 0.705 |
| | | | | | | | B-D | 0.899 |
| | | | | | | | B-E | 0.739 |
| | | | | | | | B-F | 0.899 |
| | | | | | | | B-G | 0.130 |
| | | | | | | | C-D | 0.613 |
| | | | | | | | C-E | 0.961 |
| | | | | | | | C-F | 0.613 |
| | | | | | | | C-G | 0.060 |
| | | | | | | | D-E | 0.645 |
| | | | | | | | D-F | 1.000 |
| | | | | | | | D-G | 0.165 |
| | | | | | | | E-F | 0.645 |
| | | | | | | | E-G | 0.065 |
| | | | | | | | F-G | 0.165 |
| 7 HOURS | | | | | | | | |
| A) Placebo | 31 | 0.03 | 1.08 | | 0.00 | | TRT | 0.005** |
| B) MS 30 mg | 30 | 0.67 | 0.99 | | 1.00 | | A-B | 0.017* |
| C) MS 60 mg | 30 | 0.63 | 1.00 | | 1.00 | | A-C | 0.023* |
| D) MS 90 mg | 30 | 0.77 | 1.07 | | 1.00 | | A-D | 0.006** |
| E) MS 30 mg/NTX 0.1 mg | 31 | 0.58 | 1.09 | | 0.00 | | A-E | 0.036* |
| F) MS 60 mg/NTX 0.1 mg | 30 | 0.73 | 1.05 | | 0.50 | | A-F | 0.008** |
| G) MS 90 mg/NTX 0.1 mg | 28 | 1.18 | 0.86 | | 1.00 | | A-G | <0.001*** |
| | | | | | | | B-C | 0.900 |
| | | | | | | | B-D | 0.706 |
| | | | | | | | B-E | 0.744 |
| | | | | | | | B-F | 0.801 |
| | | | | | | | B-G | 0.059 |
| | | | | | | | C-D | 0.615 |
| | | | | | | | C-E | 0.841 |
| | | | | | | | C-F | 0.706 |
| | | | | | | | C-G | 0.044* |
| | | | | | | | D-E | 0.480 |
| | | | | | | | D-F | 0.900 |
| | | | | | | | D-G | 0.128 |
| | | | | | | | E-F | 0.562 |
| | | | | | | | E-G | 0.026* |
| | | | | | | | F-G | 0.100 |
| 8 HOURS | | | | | | | | |
| A) Placebo | 31 | 0.03 | 1.08 | | 0.00 | | TRT | 0.002** |
| B) MS 30 mg | 30 | 0.57 | 0.94 | | 1.00 | | A-B | 0.041* |
| C) MS 60 mg | 30 | 0.70 | 1.09 | | 1.00 | | A-C | 0.011* |
| D) MS 90 mg | 30 | 0.73 | 1.05 | | 1.00 | | A-D | 0.008** |
| E) MS 30 mg/NTX 0.1 mg | 31 | 0.52 | 1.00 | | 0.00 | | A-E | 0.062 |
| F) MS 60 mg/NTX 0.1 mg | 30 | 0.70 | 1.06 | | 0.00 | | A-F | 0.011* |
| G) MS 90 mg/NTX 0.1 mg | 28 | 1.21 | 0.88 | | 1.00 | | A-G | <0.001*** |
| | | | | | | | B-C | 0.612 |
| | | | | | | | B-D | 0.526 |
| | | | | | | | B-E | 0.846 |
| | | | | | | | B-F | 0.612 |
| | | | | | | | B-G | 0.016* |
| | | | | | | | C-D | 0.899 |
| | | | | | | | C-E | 0.480 |
| | | | | | | | C-F | 1.000 |
| | | | | | | | C-G | 0.055 |
| | | | | | | | D-E | 0.405 |
| | | | | | | | D-F | 0.899 |
| | | | | | | | D-G | 0.073 |
| | | | | | | | E-F | 0.480 |
| | | | | | | | E-G | 0.009** |
| | | | | | | | F-G | 0.055 |

NOTE:
P-VALUES ARE FROM ONE-WAY ANALYSIS OF VARIANCE.

Tables 88A and 88B present the mean maximum pain relief (MAXPAR) and mean peak pain intensity difference (PEAKPID) scores. The mean MAXPAR scores presented in Table 88A varied among treatment groups. The mean MAXPAR score was highest for the 90 mg MS/0.1 mg NTX combination treatment group compared to all other groups. The mean scores for all 6 active treatment groups were greater than the mean score for the placebo group. The mean PEAKPID scores presented in Table 88B varied among treatment groups, and were greater for all 6 active treatment groups compared to the placebo group. Compared to all other groups, the mean PEAKPID scores were highest for the 90 mg MS/0.1 mg NTX combination treatment group.

TABLE 88A
Maximum Pain Relief Score (MAXPAR)
Primary Efficacy Population

| MAXIMUM PAIN RELIEF SCORE (MAXPAR) | | | | | | | | |
|---|---|---|---|---|---|---|---|---|
| TREATMENT | N | MEAN | SD | MIN | MEDIAN | MAX | SOURCE | P-VALUE |
| A) Placebo | 31 | 1.03 | 1.33 | | 1.00 | | TRT | <0.001*** |
| B) MS 30 mg | 30 | 2.00 | 1.29 | | 2.00 | | A-B | 0.005** |
| C) MS 60 mg | 30 | 2.13 | 1.31 | | 2.00 | | A-C | 0.002** |
| D) MS 90 mg | 30 | 2.10 | 1.45 | | 3.00 | | A-D | 0.002** |
| E) MS 30 mg/NTX 0.1 mg | 31 | 1.77 | 1.45 | | 2.00 | | A-E | 0.030* |
| F) MS 60 mg/NTX 0.1 mg | 30 | 1.97 | 1.43 | | 2.50 | | A-F | 0.007** |
| G) MS 90 mg/NTX 0.1 mg | 28 | 2.79 | 1.07 | | 3.00 | | A-G | <0.001*** |
| | | | | | | | B-C | 0.700 |
| | | | | | | | B-D | 0.773 |
| | | | | | | | B-E | 0.511 |
| | | | | | | | B-F | 0.923 |
| | | | | | | | B-G | 0.027* |
| | | | | | | | C-D | 0.923 |
| | | | | | | | C-E | 0.296 |
| | | | | | | | C-F | 0.630 |
| | | | | | | | C-G | 0.065 |
| | | | | | | | D-E | 0.343 |
| | | | | | | | D-F | 0.700 |
| | | | | | | | D-G | 0.053 |
| | | | | | | | E-F | 0.575 |
| | | | | | | | E-G | 0.004** |
| | | | | | | | F-G | 0.021* |

NOTE:
P-VALUES ARE FROM ONE-WAY ANALYSIS OF VARIANCE.

TABLE 88B
Pain Intensity Difference Score (Categorical)
Primary Efficacy Population

| PAIN INTENSITY DIFFERENCE SCORE (Categorical) | | | | | | | | |
|---|---|---|---|---|---|---|---|---|
| TREATMENT | N | MEAN | SD | MIN | MEDIAN | MAX | SOURCE | P-VALUE |
| A) Placebo | 31 | 0.35 | 0.98 | | 0.00 | | TRT | 0.006** |
| B) MS 30 mg | 30 | 0.87 | 0.90 | | 1.00 | | A-B | 0.039* |
| C) MS 60 mg | 30 | 0.97 | 1.03 | | 1.00 | | A-C | 0.014* |
| D) MS 90 mg | 30 | 1.00 | 1.08 | | 1.00 | | A-D | 0.010** |
| E) MS 30 mg/NTX 0.1 mg | 31 | 0.74 | 1.00 | | 0.00 | | A-E | 0.115 |
| F) MS 60 mg/NTX 0.1 mg | 30 | 1.00 | 0.87 | | 1.00 | | A-F | 0.010** |
| G) MS 90 mg/NTX 0.1 mg | 28 | 1.39 | 0.83 | | 2.00 | | A-G | <0.001*** |
| | | | | | | | B-C | 0.688 |
| | | | | | | | B-D | 0.592 |
| | | | | | | | B-E | 0.613 |
| | | | | | | | B-F | 0.592 |
| | | | | | | | B-G | 0.039* |
| | | | | | | | C-D | 0.893 |
| | | | | | | | C-E | 0.363 |
| | | | | | | | C-F | 0.893 |
| | | | | | | | C-G | 0.094 |
| | | | | | | | D-E | 0.296 |
| | | | | | | | D-F | 1.000 |
| | | | | | | | D-G | 0.122 |
| | | | | | | | E-F | 0.296 |
| | | | | | | | E-G | 0.010* |
| | | | | | | | F-G | 0.122 |

NOTE:
P-VALUES ARE FROM ONE-WAY ANALYSIS OF VARIANCE.

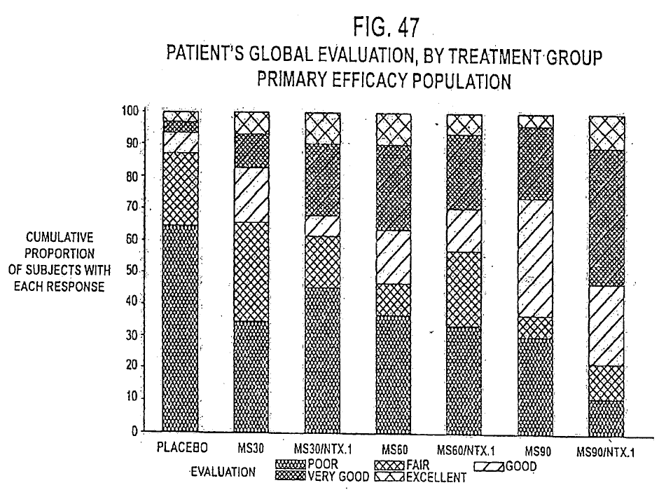

Table 89 presents the summary and analysis of global evaluations (see also FIG. 47). The placebo treatment group had the highest number of subjects who had “poor” global evaluation scores. The 90 mg MS/0.1 mg NTX combination treatment group had the highest number of subjects with a total of “excellent”, “very good” and “good” global evaluation scores. The profiles of the global evaluation scores are based on subjects' evaluations.

TABLE 89
Global Evaluation of Study Medication
Primary Efficacy Population

| | | | | | Very | | | | | | |
|---|---|---|---|---|---|---|---|---|---|---|---|
| | | Poor | Fair | Good | Good | Excellent | | | | | |
| TREATMENT | N | (0) | (1) | (2) | (3) | (4) | Mean | (SD) | Median | Source | P-Value |
| A) Placebo | 31 | 20 (64.5%) | 7 (22.6%) | 2 (6.5%) | 1 (3.2%) | 1 (3.2%) | 0.58 | 0.99 | 0.00 | TRT | <0.001*** |
| B) MS 30 mg | 29 | 10 (34.5%) | 9 (31.0%) | 5 (17.2%) | 3 (10.3%) | 2 (6.9%) | 1.24 | 1.24 | 1.00 | A-B | 0.049* |
| C) MS 60 mg | 30 | 11 (36.7%) | 3 (10.0%) | 5 (16.7%) | 8 (26.7%) | 3 (10.0%) | 1.63 | 1.47 | 2.00 | A-C | 0.002** |
| D) MS 90 mg | 30 | 9 (30.0%) | 2 (6.7%) | 11 (36.7%) | 7 (23.3%) | 1 (3.3%) | 1.63 | 1.25 | 2.00 | A-D | 0.002** |
| E) MS 30 mg/NTX 0.1 mg | 31 | 14 (45.2%) | 5 (16.1%) | 2 (6.5%) | 7 (22.6%) | 3 (9.7%) | 1.35 | 1.50 | 1.00 | A-E | 0.019* |
| F) MS 60 mg/NTX 0.1 mg | 30 | 10 (33.3%) | 7 (23.3%) | 4 (13.3%) | 7 (23.3%) | 2 (6.7%) | 1.47 | 1.36 | 1.00 | A-F | 0.008** |
| G) MS 90 mg/NTX 0.1 mg | 28 | 3 (10.7%) | 3 (10.7%) | 7 (25.0%) | 12 (42.9%) | 3 (10.7%) | 2.32 | 1.16 | 3.00 | A-G | <0.001*** |
| | | | | | | | | | | B-C | 0.246 |
| | | | | | | | | | | B-D | 0.246 |
| | | | | | | | | | | B-E | 0.734 |
| | | | | | | | | | | B-F | 0.504 |
| | | | | | | | | | | B-G | 0.002** |
| | | | | | | | | | | C-D | 1.000 |
| | | | | | | | | | | C-E | 0.401 |
| | | | | | | | | | | C-F | 0.618 |
| | | | | | | | | | | C-G | 0.044* |
| | | | | | | | | | | D-E | 0.401 |
| | | | | | | | | | | D-F | 0.618 |
| | | | | | | | | | | D-G | 0.044* |
| | | | | | | | | | | E-F | 0.736 |
| | | | | | | | | | | E-G | 0.005** |
| | | | | | | | | | | F-G | 0.013* |

NOTE:
P-VALUES ARE FROM ONE-WAY ANALYSIS OF VARIANCE

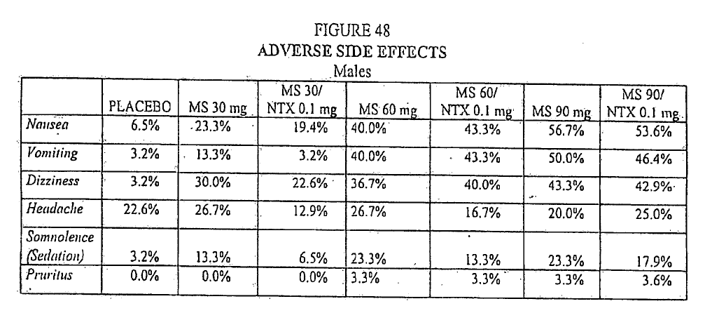

The majority of adverse events reported were categorized as digestive (nausea or vomiting) or nervous system (dizziness or somnolence) as further shown in Table 90. FIG. 48 represents a summary of exemplary adverse side effects that may be attenuated according to methods and compositions of the invention.

TABLE 90
Adverse Events Primary Efficacy Population

| | | Total | No. Of | | | Total | | | |
|---|---|---|---|---|---|---|---|---|---|
| Body System | | No. Of | Patients | | | No. Of | Severity | | |
| Adverse Events | Treatment | Patients | W/Event | Source | P-Value | Events | Mild | Moderate | Severe |
| ALL BODY SYSTEMS | PLACEBO | 31 | 9 (29.0%) | TRT | <0.001*** | 21 | 9 (42.9%) | 7 (33.3%) | 5 (23.8%) |
| ALL EVENTS | MS30 | 30 | 20 (66.7%) | A-B | 0.003** | 57 | 21 (36.8%) | 25 (43.9%) | 11 (19.3%) |
| | MS60 | 30 | 27 (90.0%) | A-C | <0.001*** | 83 | 44 (53.0%) | 27 (32.5%) | 12 (14.5%) |
| | MS90 | 30 | 28 (93.3%) | A-D | <0.001*** | 108 | 47 (43.5%) | 39 (36.1%) | 22 (20.4%) |
| | MS30/NTX.1 | 31 | 17 (54.8%) | A-E | 0.039* | 34 | 14 (41.2%) | 17 (50.0%) | 3 (8.8%) |
| | MS60/NTX.1 | 30 | 24 (80.0%) | A-F | <0.001*** | 80 | 31 (38.8%) | 35 (43.8%) | 14 (17.5%) |
| | MS90/NTX.1 | 28 | 24 (85.7%) | A-G | <0.001*** | 79 | 39 (49.4%) | 26 (32.9%) | 14 (17.7%) |
| | | 100 | | B-C | 0.028* | | | | |
| | | | | B-D | 0.010** | | | | |
| | | | | C-E | 0.002** | | | | |
| | | | | D-E | <0.001*** | | | | |
| | | | | E-F | 0.036* | | | | |
| | | | | E-G | 0.010* | | | | |
| CARDIAC DISORDERS | PLACEBO | 31 | 0 (0.0%) | TRT | 0.420 | 0 | 0 | 0 | 0 |
| ALL EVENTS | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.0%) | | | 1 | 0 (0.0%) | 1 (100.0%) | 0 (0.0%) |
| | MS90/NTX.1 | 28 | 0 (0.0%) | | | 0 | 0 | 0 | 0 |
| CHEST PRESSURE | PLACEBO | 31 | 0 (0.0%) | TRT | 0.420 | 0 | 0 | 0 | 0 |
| SENSATION | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| EAR AND LABYRINTH | PLACEBO | 31 | 0 (0.0%) | TRT | 0.552 | 0 | 0 | 0 | 0 |
| DISORDERS | MS30 | 30 | 0 (0.0%) | | | 0 | 0 | 0 | 0 |
| ALL EVENTS | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 1 (3.2%) | | | 1 | 0 (0.0%) | 1 (100.0%) | 0 (0.0%) |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| SENSATION OF | PLACEBO | 31 | 0 (0.0%) | TRT | 0.420 | 0 | 0 | 0 | 0 |
| PRESSURE IN EAR | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| TINNITUS | PLACEBO | 31 | 0 (0.0%) | TRT | 0.446 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 1 (3.2%) | | | 1 | 0 (0.0%) | 1 (100.0%) | 0 (0.0%) |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| EYE DISORDERS | PLACEBO | 31 | 2 (6.5%) | TRT | 0.175 | 2 | 1 (50.0%) | 1 (50.0%) | 0 (0.0%) |
| ALL EVENTS | MS30 | 30 | 6 (20.0%) | A-C | 0.033* | 6 | 3 (50.0%) | 3 (50.0%) | 0 (0.0%) |
| | MS60 | 30 | 8 (26.7%) | A-D | 0.017* | 8 | 5 (62.5%) | 1 (12.5%) | 2 (25.0%) |
| | MS90 | 30 | 9 (30.0%) | A-G | 0.048* | 11 | 8 (72.7%) | 2 (18.2%) | 1 (9.1%) |
| | MS30/NTX.1 | 31 | 3 (9.7%) | D-E | 0.046* | 3 | 2 (66.7%) | 1 (33.3%) | 0 (0.0%) |
| | MS60/NTX.1 | 30 | 7 (23.3%) | | | 7 | 2 (28.6%) | 5 (71.4%) | 0 (0.0%) |
| | MS90/NTX.1 | 28 | 7 (25.0%) | | | 9 | 6 (66.7%) | 3 (33.3%) | 0 (0.0%) |
| BLOODSHOT EYE | PLACEBO | 31 | 2 (6.5%) | TRT | 0.175 | 2 | 1 (50.0%) | 1 (50.0%) | 0 (0.0%) |
| | MS30 | 30 | 6 (20.0%) | A-C | 0.033* | 6 | 3 (50.0%) | 3 (50.0%) | 0 (0.0%) |
| | MS60 | 30 | 8 (26.7%) | A-D | 0.017* | 8 | 5 (62.5%) | 1 (12.5%) | 2 (25.0%) |
| | MS90 | 30 | 9 (30.0%) | A-G | 0.048* | 9 | 7 (77.8%) | 1 (11.1%) | 1 (11.1%) |
| | MS30/NTX.1 | 31 | 3 (9.7%) | D-E | 0.046* | 3 | 2 (66.7%) | 1 (33.3%) | 0 (0.0%) |
| | MS60/NTX.1 | 30 | 7 (23.3%) | | | 7 | 2 (28.6%) | 5 (71.4%) | 0 (0.0%) |
| | MS90/NTX.1 | 28 | 7 (25.0%) | | | 7 | 5 (71.4%) | 2 (28.6%) | 0 (0.0%) |
| EYE IRRITATION | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| EYE PAIN | PLACEBO | 31 | 0 (0.0%) | TRT | 0.366 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| MIOSIS | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| PHOTOPHOBIA | PLACEBO | 31 | 0 (0.0%) | TRT | 0.366 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 0 (0.0%) | 1 (100.0%) | 0 (0.0%) |

GASTROINTESTINAL DISORDERS

| | | | | | | | | | |
|---|---|---|---|---|---|---|---|---|---|
| ALL EVENTS | PLACEBO | 31 | 2 (6.5%) | TRT | <0.001*** | 3 | 2 (66.7%) | 0 (0.0%) | 1 (33.3%) |
| | MS30 | 30 | 10 (33.3%) | A-B | 0.008** | 14 | 4 (28.6%) | 5 (35.7%) | 5 (35.7%) |
| | MS60 | 30 | 15 (50.0%) | A-C | <0.001*** | 29 | 12 (41.4%) | 8 (27.6%) | 9 (31.0%) |
| | MS90 | 30 | 19 (63.3%) | A-D | <0.001*** | 42 | 11 (26.2%) | 18 (42.9%) | 13 (31.0%) |
| | MS30/NTX.1 | 31 | 7 (22.6%) | A-F | <0.001*** | 8 | 3 (37.5%) | 4 (50.0%) | 1 (12.5%) |
| | MS60/NTX.1 | 30 | 16 (53.3%) | A-G | <0.001*** | 33 | 7 (21.2.%) | 15 (45.5%) | 11 (33.3%) |
| | MS90/NTX.1 | 28 | 18 (64.3%) | B-D | 0.020* | 32 | 9 (28.1%) | 11 (34.4%) | 12 (37.5%) |
| | | | | B-G | 0.018* | | | | |
| | | | | C-E | 0.026* | | | | |
| | | | | D-E | 0.001** | | | | |
| | | | | E-F | 0.013* | | | | |
| | | | | E-G | 0.001** | | | | |
| ABDOMINAL PAIN NOS | PLACEBO | 31 | 0 (0.0%) | TRT | 0.059 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 2 (6.7%) | | | 2 | 0 (0.0%) | 1 (50.0%) | 1 (50.0%) |
| | MS90/NTX.1 | 28 | 0 (0.0%) | | | 0 | 0 | 0 | 0 |
| ABDOMINAL PAIN | PLACEBO | 31 | 0 (0.0%) | TRT | 0.420 | 0 | 0 | 0 | 0 |
| LOWER | MS30 | 30 | 1 (3.3%) | | | 1 | 0 (0.0%) | 1 (100.0%) | 0 (0.0%) |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| ABDOMINAL PAIN | PLACEBO | 31 | 0 (0.0%) | TRT | 0.366 | 0 | 0 | 0 | 0 |
| UPPER | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| DRY MOUTH | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| DRY THROAT | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| DYSPEPSIA | PLACEBO | 31 | 0 (0.0%) | TRT | 0.176 | 0 | 0 | 0 | 0 |
| | MS30 | 30 | 0 (0.0%) | | | 0 | 0 | 0 | 0 |
| | MS60 | 30 | 2 (6.7%) | | | 2 | 1 (50.0%) | 1 (50.0%) | 0 (0.0%) |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| DYSPHAGIA | PLACEBO | 31 | 0 (0.0%) | TRT | 0.669 | 0 | 0 | 0 | 0 |
| | MS30 | 30 | 1 (3.3%) | | | 1 | 0 (0.0%) | 1 (100.0%) | 0 (0.0%) |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| HICCUPS | PLACEBO | 31 | 0 (0.0%) | TRT | 0.506 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 1 (3.6%) | | | 1 | 0 (0.0%) | 0 (0.0%) | 1 (100.0%) |
| MOUTH | PLACEBO | 31 | 0 (0.0%) | TRT | 0.366 | 0 | 0 | 0 | 0 |
| HEMORRHAGE | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| NAUSEA | PLACEBO | 31 | 2 (6.5%) | TRT | <0.001*** | 2 | 1 (50.0%) | 0 (0.0%) | 1 (50.0%) |
| | MS30 | 30 | 7 (23.3%) | A-C | 0.002** | 7 | 4 (57.1%) | 1 (14.3%) | 2 (28.6%) |
| | MS60 | 30 | 12 (40.0%) | A-D | <0.001*** | 14 | 8 (57.1%) | 4 (28.6%) | 2 (14.3%) |
| | MS90 | 30 | 17 (56.7%) | A-F | <0.001*** | 21 | 6 (28.6%) | 12 (57.1%) | 3 (14.3%) |
| | MS30/NTX.1 | 31 | 6 (19.4%) | A-G | <0.001*** | 6 | 2 (33.3%) | 3 (50.0%) | 1 (16.7%) |
| | MS60/NTX.1 | 30 | 13 (43.3%) | B-D | 0.008** | 15 | 5 (33.3%) | 8 (53.3%) | 2 (13.3%) |
| | MS90/NTX.1 | 28 | 15 (53.6%) | B-G | 0.018* | 15 | 4 (26.7%) | 9 (60.0%) | 2 (13.3%) |
| | | | | D-E | 0.003** | | | | |
| | | | | E-F | 0.043* | | | | |
| | | | | E-G | 0.006** | | | | |
| PARAESTHESIA LIPS | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| RETCHING | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 0 (0.0%) | 0 (0.0%) | 1 (100.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| SORE THROAT NOS | PLACEBO | 31 | 0 (0.0%) | TRT | 0.809 | 0 | 0 | 0 | 0 |
| | MS30 | 30 | 1 (3.3%) | | | 1 | 0 (0.0%) | 0 (0.0%) | 1 (100.0%) |
| | MS60 | 30 | 1 (3.3%) | | | 1 | 1 (100.0%) | 0 (0.0%) | 0 (0.0%) |
| | MS90 | 30 | 1 (3.3%) | | | 1 | 0 (0.0%) | 1 (100.0%) | 0 (0.0%) |
| | MS30/NTX.1 | 31 | 1 (3.2%) | | | 1 | 1 (100.0%) | 0 (0.0%) | 0 (0.0%) |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| VOMITING NOS | PLACEBO | 31 | 1 (3.2%) | TRT | <0.001*** | 1 | 1 (100.0%) | 0 (0.0%) | 0 (0.0%) |
| | MS30 | 30 | 4 (13.3%) | A-C | <0.001*** | 4 | 0 (0.0%) | 2 (50.0%) | 2 (50.0%) |
| | MS60 | 30 | 12 (40.0%) | A-D | <0.001*** | 12 | 2 (16.7%) | 3 (25.0%) | 7 (58.3%) |
| | MS90 | 30 | 15 (50.0%) | A-F | <0.001*** | 16 | 2 (12.5%) | 5 (31.3%) | 9 (56.3%) |
| | MS30/NTX.1 | 31 | 1 (3.2%) | A-G | <0.001*** | 1 | 0 (0.0%) | 1 (100.0%) | 0 (0.0%) |
| | MS60/NTX.1 | 30 | 13 (43.3%) | B-C | 0.020* | 13 | 2 (15.4%) | 3 (23.1%) | 8 (61.5%) |
| | MS90/NTX.1 | 28 | 13 (46.4%) | B-D | 0.002** | 13 | 2 (15.4%) | 2 (15.4%) | 9 (69.2%) |
| | | | | B-F | 0.010** | | | | |
| | | | | B-G | 0.006** | | | | |
| | | | | C-E | <0.001*** | | | | |
| | | | | D-E | <0.001*** | | | | |
| | | | | E-F | <0.001*** | | | | |
| | | | | E-G | <0.001*** | | | | |

GENERAL DISORDERS AND ADMINISTRATION SITE CONDITIONS

| | | | | | | | | | |
|---|---|---|---|---|---|---|---|---|---|
| ALL EVENTS | PLACEBO | 31 | 1 (3.2%) | TRT | 0.739 | 1 | 1 (100.0%) | 0 (0.0%) | 0 (0.0%) |
| | MS30 | 30 | 5 (16.7%) | | | 6 | 1 (16.7%) | 4 (66.7%) | 1 (16.7%) |
| | MS60 | 30 | 4 (13.3%) | | | 4 | 1 (25.0%) | 3 (75.0%) | 0 (0.0%) |
| | MS90 | 30 | 4 (13.3%) | | | 9 | 2 (22.2%) | 5 (55.6%) | 2 (22.2%) |
| | MS30/NTX.1 | 31 | 4 (12.9%) | | | 4 | 2 (50.0%) | 2 (50.0%) | 0 (0.0%) |
| | MS60/NTX.1 | 30 | 5 (16.7%) | | | 6 | 3 (50.0%) | 3 (50.0%) | 0 (0.0%) |
| | MS90/NTX.1 | 28 | 3 (10.7%) | | | 3 | 1 (33.3%) | 2 (66.7%) | 0 (0.0%) |
| ENERGY | PLACEBO | 31 | 0 (0.0%) | TRT | 0.420 | 0 | 0 | 0 | 0 |
| INCREASED | MS30 | 30 | 0 (0.0%) | | | 0 | 0 | 0 | 0 |
| | MS60 | 30 | 1 (3.3%) | | | 1 | 0 (0.0%) | 1 (0.0%) | 0 (0.0%) |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| FATIGUE | PLACEBO | 31 | 0 (0.0%) | TRT | 0.312 | 0 | 0 | 0 | 0 |
| | MS30 | 30 | 1 (3.3%) | A-D | 0.035* | 1 | 0 (0.0%) | 1 (100.0%) | 0 (0.0%) |
| | MS60 | 30 | 1 (3.3%) | | | 1 | 0 (0.0%) | 1 (100.0%) | 0 (0.0%) |
| | MS90 | 30 | 4 (13.3%) | | | 5 | 0 (0.0%) | 4 (80.0%) | 1 (20.0%) |
| | MS30/NTX.1 | 31 | 2 (6.5%) | | | 2 | 0 (0.0%) | 2 (100.0%) | 0 (0.0%) |
| | MS60/NTX.1 | 30 | 1 (3.3%) | | | 1 | 0 (0.0%) | 1 (100.0%) | 0 (0.0%) |
| | MS90/NTX.1 | 28 | 1 (3.6%) | | | 1 | 0 (0.0%) | 1 (100.0%) | 0 (0.0%) |
| FEELING HOT | PLACEBO | 31 | 1 (3.2%) | TRT | 0.835 | 1 | 1 (100.0%) | 0 (0.0%) | 0 (0.0%) |
| | MS30 | 30 | 2 (6.7%) | | | 2 | 1 (50.0%) | 0 (0.0%) | 1 (50.0%) |
| | MS60 | 30 | 0 (0.0%) | | | 0 | 0 | 0 | 0 |
| | MS90 | 30 | 2 (6.7%) | | | 2 | 2 (100.0%) | 0 (0.0%) | 0 (0.0%) |
| | MS30/NTX.1 | 31 | 1 (3.2%) | | | 1 | 1 (100.0%) | 0 (0.0%) | 0 (0.0%) |
| | MS60/NTX.1 | 30 | 2 (6.7%) | | | 2 | 1 (50.0%) | 1 (50.0%) | 0 (0.0%) |
| | MS90/NTX.1 | 28 | 1 (3.6%) | | | 1 | 1 (100.0%) | 0 (0.0%) | 0 (0.0%) |
| FEELING JITTERY | PLACEBO | 31 | 0 (0.0%) | TRT | 0.538 | 0 | 0 | 0 | 0 |
| | MS30 | 30 | 0 (0.0%) | | | 0 | 0 | 0 | 0 |
| | MS60 | 30 | 1 (3.3%) | | | 1 | 1 (100.0%) | 0 (0.0%) | 0 (0.0%) |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |
| NECK SWELLING | PLACEBO | 31 | 0 (0.0%) | TRT | 0.366 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 0 (0.0%) | 1 (100.0%) | 0 (0.0%) |
| PYREXIA | PLACEBO | 31 | 0 (0.0%) | TRT | 0.538 | 0 | 0 | 0 | 0 |
| | MS30 | 30 | 1 (3.3%) | | | 1 | 0 (0.0%) | 1 (100.0%) | 0 (0.0%) |
| | MS60 | 30 | 0 (0.0%) | | | 0 | 0 | 0 | 0 |
| | MS90 | 30 | 1 (3.3%) | | | 1 | 0 (0.0%) | 0 (0.0%) | 1 (100.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| SHIVERING | PLACEBO | 31 | 0 (0.0%) | TRT | 0.679 | 0 | 0 | 0 | 0 |
| | MS30 | 30 | 1 (3.3%) | | | 1 | 0 (0.0%) | 1 (100.0%) | 0 (0.0%) |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 1 (3.2%) | | | 1 | 1 (100.0%) | 0 (0.0%) | 0 (0.0%) |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |
| WEAKNESS | PLACEBO | 31 | 0 (0.0%) | TRT | 0.802 | 0 | 0 | 0 | 0 |
| | MS30 | 30 | 1 (3.3%) | | | 1 | 0 (0.0%) | 1 (100.0%) | 0 (0.0%) |
| | MS60 | 30 | 1 (3.3%) | | | 1 | 0 (0.0%) | 1 (100.0%) | 0 (0.0%) |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| INVESTIGATIONS | PLACEBO | 31 | 0 (0.0%) | TRT | 0.363 | 0 | 0 | 0 | 0 |
| ALL EVENTS | MS30 | 30 | 2 (6.7%) | | | 2 | 2 (100.0%) | 0 (0.0%) | 0 (0.0%) |
| | MS60 | 30 | 1 (3.3%) | | | 1 | 1 (100.0%) | 0 (0.0%) | 0 (0.0%) |
| | MS90 | 30 | 0 (0.0%) | | | 0 | 0 | 0 | 0 |
| | MS30/NTX.1 | 31 | 1 (3.2%) | | | 1 | 0 (0.0%) | 1 (100.0%) | 0 (0.0%) |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| BLOOD PRESSURE | PLACEBO | 31 | 0 (0.0%) | TRT | 0.420 | 0 | 0 | 0 | 0 |
| INCREASED | MS30 | 30 | 0 (0.0%) | | | 0 | 0 | 0 | 0 |
| | MS60 | 30 | 1 (3.3%) | | | 1 | 1 (100.0%) | 0 (0.0%) | 0 (0.0%) |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| BODY | PLACEBO | 31 | 0 (0.0%) | TRT | .059 | 0 | 0 | 0 | 0 |
| TEMPERATURE | MS30 | 30 | 2 (6.7%) | | | 2 | 2 (100.0%) | 0 (0.0%) | 0 (0.0%) |
| INCREASED | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| HEART RATE | PLACEBO | 31 | 0 (0.0%) | TRT | 0.446 | 0 | 0 | 0 | 0 |
| INCREASED | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 1 (3.2%) | | | 1 | 0 (0.0%) | 1 (100.0%) | 0 (0.0%) |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |

MUSCULOSKELETAL CONNECTIVE TISSUE AND BONE DISORDERS

| | | | | | | | | | |
|---|---|---|---|---|---|---|---|---|---|
| ALL EVENTS | PLACEBO | 31 | 1 (3.2%) | TRT | 0.679 | 1 | 0 (0.0%) | 1 (100.0%) | 0 (0.0%) |
| | MS30 | 30 | 0 (0.0%) | | | 0 | 0 | 0 | 0 |
| | MS60 | 30 | 1 (3.3%) | | | 2 | 0 (0.0%) | 2 (100.0%) | 0 (0.0%) |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| JOINT RANGE OF | PLACEBO | 31 | 1 (3.2%) | TRT | 0.446 | 1 | 0 (0.0%) | 1 (100.0%) | 0 (0.0%) |
| MOTION | MS30 | 30 | 0 (0.0%) | | | 0 | 0 | 0 | 0 |
| DECREASED | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| MUSCLE SPASMS | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| MYALGIA | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 1 (3.3%) | | | 2 | 0 (0.0%) | 2 (100.0%) | 0 (0.0%) |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |

NERVOUS SYSTEM DISORDERS

| | | | | | | | | | |
|---|---|---|---|---|---|---|---|---|---|
| ALL EVENTS | PLACEBO | 31 | 7 (22.6%) | TRT | <0.001*** | 14 | 5 (35.7%) | 5 (35.7%) | 4 (28.6%) |
| | MS30 | 30 | 15 (50.0%) | A-B | 0.026* | 23 | 8 (34.8%) | 11 (47.8%) | 4 (17.4%) |
| | MS60 | 30 | 21 (70.0%) | A-C | <0.001*** | 29 | 16 (55.2%) | 12 (41.4%) | 1 (3.4%) |
| | MS90 | 30 | 19 (63.3%) | A-D | <0.001*** | 31 | 17 (54.8%) | 9 (29.0%) | 5 (16.1%) |
| | MS30/NTX.1 | 31 | 11 (35.5%) | A-F | 0.048* | 15 | 7 (46.7%) | 6 (40.0%) | 2 (13.3%) |
| | MS60/NTX.1 | 30 | 14 (46.7%) | A-G | <0.001*** | 25 | 13 (52.0%) | 9 (36.0%) | 3 (12.0%) |
| | MS90/NTX.1 | 28 | 19 (67.9%) | C-E | 0.007** | 28 | 18 (64.3%) | 8 (28.6%) | 2 (7.1%) |
| | | | | D-E | 0.030* | | | | |
| | | | | E-G | 0.013* | | | | |
| DIZZINESS (EXC | PLACEBO | 31 | 1 (3.2%) | TRT | 0.007** | 1 | 0 (0.0%) | 0 (0.0%) | 1 (100.0%) |
| VERTIGO) | MS30 | 30 | 9 (30.0%) | A-B | 0.005** | 10 | 5 (50.0%) | 3 (30.0%) | 2 (20.0%) |
| | MS60 | 30 | 11 (36.7%) | A-C | 0.001** | 12 | 7 (58.3%) | 5 (41.7%) | 0 (0.0%) |
| | MS90 | 30 | 13 (43.3%) | A-D | <0.001*** | 14 | 9 (64.3%) | 4 (28.6%) | 1 (7.1%) |
| | MS30/NTX.1 | 31 | 7 (22.6%) | A-E | 0.023* | 8 | 3 (37.5%) | 4 (50.0%) | 1 (12.5%) |
| | MS60/NTX.1 | 30 | 12 (40.0%) | A-F | <0.001*** | 12 | 7 (58.3%) | 4 (33.3%) | 1 (8.3%) |
| | MS90/NTX.1 | 28 | 12 (42.9%) | A-G | <0.001*** | 14 | 8 (57.1%) | 4 (28.6%) | 2 (14.3%) |
| HEADACHE NOS | PLACEBO | 31 | 7 (22.6%) | TRT | 0.810 | 9 | 4 (44.4%) | 2 (22.2%) | 3 (33.3%) |
| | MS30 | 30 | 8 (26.7%) | | | 8 | 1 (12.5%) | 5 (62.5%) | 2 (25.0%) |
| | MS60 | 30 | 8 (26.7%) | | | 10 | 6 (60.0%) | 4 (40.0%) | 0 (0.0%) |
| | MS90 | 30 | 6 (20.0%) | | | 6 | 5 (83.3%) | 1 (16.7%) | 0 (0.0%) |
| | MS30/NTX.1 | 31 | 4 (12.9%) | | | 4 | 3 (75.0%) | 1 (25.0%) | 0 (0.0%) |
| | MS60/NTX.1 | 30 | 5 (16.7%) | | | 5 | 2 (40.0%) | 2 (40.0%) | 1 (20.0%) |
| | MS90/NTX.1 | 28 | 7 (25.0%) | | | 7 | 6 (85.7%) | 1 (14.3%) | 0 (0.0%) |
| HYPERAESTHESIA | PLACEBO | 31 | 1 (3.2%) | TRT | 0.446 | 1 | 0 (0.0%) | 1 (100.0%) | 0 (0.0%) |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| HYPOAESTHESIA | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |
| PARAESTHESIA | PLACEBO | 31 | 0 (0.0%) | TRT | 0.506 | 0 | 0 | 0 | 0 |
| NEC | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 1 (3.6%) | | | 2 | 2 (100.0%) | 0 (0.0%) | 0 (0.0%) |
| SOMNOLENCE | PLACEBO | 31 | 1 (3.2%) | TRT | 0.174 | 1 | 0 (0.0%) | 1 (100.0%) | 0 (0.0%) |
| | MS30 | 30 | 4 (13.3%) | A-C | 0.020* | 5 | 2 (40.0%) | 3 (60.0%) | 0 (0.0%) |
| | MS60 | 30 | 7 (23.3%) | A-D | 0.020* | 7 | 3 (42.9%) | 3 (42.9%) | 1 (14.3%) |
| | MS90 | 30 | 7 (23.3%) | | | 7 | 2 (28.6%) | 4 (57.1%) | 1 (14.3%) |
| | MS30/NTX.1 | 31 | 2 (6.5%) | | | 2 | 0 (0.0%) | 1 (50.0%) | 1 (50.0%) |
| | MS60/NTX.1 | 30 | 4 (13.3%) | | | 5 | 1 (20.0%) | 3 (60.0%) | 1 (20.0%) |
| | MS90/NTX.1 | 28 | 5 (17.9%) | | | 5 | 2 (40.0%) | 3 (60.0%) | 0 (0.0%) |
| SYNCOPE | PLACEBO | 31 | 1 (3.2%) | TRT | 0.368 | 1 | 0 (0.0%) | 1 (100.0%) | 0 (0.0%) |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 2 (6.7%) | | | 2 | 0 (0.0%) | 0 (0.0%) | 2 (100.0%) |
| | MS30/NTX.1 | 31 | 1 (3.2%) | | | 1 | 1 (100.0%) | 0 (0.0%) | 0 (0.0%) |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| TENSION | PLACEBO | 31 | 1 (3.2%) | TRT | 0.446 | 1 | 1 (100.0%) | 0 (0.0%) | 0 (0.0%) |
| HEADACHES | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| TREMOR NEC | PLACEBO | 31 | 0 (0.0%) | TRT | 0.186 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 2 (6.7%) | | | 2 | 1 (50.0%) | 0 (0.0%) | 1 (50.0%) |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |

PSYCHIATRIC DISORDERS

| | | | | | | | | | |
|---|---|---|---|---|---|---|---|---|---|
| ALL EVENTS | PLACEBO | 31 | 0 (0.0%) | TRT | 0.554 | 0 | 0 | 0 | 0 |
| | MS30 | 30 | 1 (3.3%) | | | 1 | 0 (0.0%) | 1 (100.0%) | 0 (0.0%) |
| | MS60 | 30 | 1 (3.3%) | | | 1 | 1 (100.0%) | 0 (0.0%) | 0 (0.0%) |
| | MS90 | 30 | 2 (6.7%) | | | 2 | 2 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |
| ANXIETY NEC | PLACEBO | 31 | 0 (0.0%) | TRT | 0.538 | 0 | 0 | 0 | 0 |
| | MS30 | 30 | 1 (3.3%) | | | 1 | 0 (0.0%) | 1 (100.0%) | 0 (0.0%) |
| | MS60 | 30 | 1 (3.3%) | | | 1 | 1 (100.0%) | 0 (0.0%) | 0 (0.0%) |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| EUPHORIC MOOD | PLACEBO | 31 | 0 (0.0%) | TRT | 0.59 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 2 (6.7%) | | | 2 | 2 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| NERVOUSNESS | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |

RENAL AND URINARY DISORDERS

| | | | | | | | | | |
|---|---|---|---|---|---|---|---|---|---|
| ALL EVENTS | PLACEBO | 31 | 0 (0.0%) | TRT | 0.506 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| DIFFICULTY IN | PLACEBO | 31 | 0 (0.0%) | TRT | 0.506 | 0 | 0 | 0 | 0 |
| MICTURITION | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |

RESPIRATORY, THORACIC AND MEDIASTINAL DISORDERS

| | | | | | | | | | |
|---|---|---|---|---|---|---|---|---|---|
| ALL EVENTS | PLACEBO | 31 | 0 (0.0%) | TRT | 0.802 | 0 | 0 | 0 | 0 |
| | MS30 | 30 | 1 (3.3%) | | | 1 | 1 (100.0%) | 0 (0.0%) | 0 (0.0%) |
| | MS60 | 30 | 1 (3.3%) | | | 1 | 1 (100.0%) | 0 (0.0%) | 0 (0.0%) |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 1 (3.3%) | | | 1 | 0 (0.0%) | 1 (100.0%) | 0 (0.0%) |
| | MS90/NTX.1 | 28 | 0 (0.0%) | | | 0 | 0 | 0 | 0 |
| CHEST TIGHTNESS | PLACEBO | 31 | 0 (0.0%) | TRT | 0.420 | 0 | 0 | 0 | 0 |
| | MS30 | 30 | 1 (3.3%) | | | 1 | 1 (100.0%) | 0 (0.0%) | 0 (0.0%) |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| DYSPNOEA NOS | PLACEBO | 31 | 0 (0.0%) | TRT | 0.538 | 0 | 0 | 0 | 0 |
| | MS30 | 30 | 0 (0.0%) | | | 0 | 0 | 0 | 0 |
| | MS60 | 30 | 1 (3.3%) | | | 1 | 1 (100.0%) | 0 (0.0%) | 0 (0.0%) |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| THROAT | PLACEBO | 31 | 0 (0.0%) | TRT | 0.420 | 0 | 0 | 0 | 0 |
| TIGHTNESS | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 1 | 0 (0.0)% | 1 (100.0%) | 0 (0.0%) |
| | MS90/NTX.1 | 28 | 0 (0.0%) | | | 0 | 0 | 0 | 0 |

SKIN & SUBCUTANEOUS TISSUE DISORDERS

| | | | | | | | | | |
|---|---|---|---|---|---|---|---|---|---|
| ALL EVENTS | PLACEBO | 31 | 0 (0.0%) | TRT | 0.213 | 0 | 0 | 0 | 0 |
| | MS30 | 30 | 3 (10.0%) | A-C | 0.018* | 3 | 2 (66.7%) | 0 (0.0%) | 1 (33.3%) |
| | MS60 | 30 | 5 (16.7%) | A-D | 0.009** | 7 | 6 (85.7%) | 1 (14.3%) | 0 (0.0%) |
| | MS90 | 30 | 6 (20.0%) | A-G | 0.029* | 7 | 5 (71.4%) | 1 (14.3%) | 1 (14.3%) |
| | MS30/NTX.1 | 31 | 2 (6.5%) | | | 2 | 0 (0.0%) | 2 (100.0%) | 0 (0.0%) |
| | MS60/NTX.1 | 30 | 3 (10.0%) | | | 5 | 5 (100.0%) | 0 (0.0%) | 0 (0.0%) |
| | MS90/NTX.1 | 28 | 4 (14.3%) | | | 4 | 2 (50.0%) | 2 (50.0%) | 0 (0.0%) |
| CLAMMINESS | PLACEBO | 31 | 0 (0.0%) | TRT | 0.538 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 0 (0.0%) | 0 (0.0%) | 1 (100.0%) |
| | MS30/NTX.1 | 31 | 0 (0.0%) | | | 0 | 0 | 0 | 0 |
| | MS60/NTX.1 | 30 | 1 (3.3%) | | | 1 | 1 (100.0%) | 0 (0.0%) | 0 (0.0%) |
| | MS90/NTX.1 | 28 | 0 (0.0%) | | | 0 | 0 | 0 | 0 |
| DERMATITIS NOS | PLACEBO | 31 | 0 (0.0%) | TRT | 0.357 | 0 | 0 | 0 | 0 |
| | MS30 | 30 | 1 (3.3%) | | | 1 | 0 (0.0%) | 0 (0.0%) | 1 (100.0%) |
| | MS60 | 30 | 2 (6.7%) | | | 2 | 2 (100.0%) | 0 (0.0%) | 0 (0.0%) |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| ECCHYMOSIS | PLACEBO | 31 | 0 (0.0%) | TRT | 0.420 | 0 | 0 | 0 | 0 |
| | MS30 | 30 | 1 (3.3%) | | | 1 | 1 (100.0%) | 0 (0.0%) | 0 (0.0%) |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| PHOTOSENSITIVITY | PLACEBO | 31 | 0 (0.0%) | TRT | 0.420 | 0 | 0 | 0 | 0 |
| REACTION NOS | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |
| PRURITUS NOS | PLACEBO | 31 | 0 (0.0%) | TRT | 0.785 | 0 | 0 | 0 | 0 |
| | MS30 | 30 | 0 (0.0%) | | | 0 | 0 | 0 | 0 |
| | MS60 | 30 | 1 (3.3%) | | | 2 | 2 (100.0%) | 0 (0.0%) | 0 (0.0%) |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 1 (3.3%) | | | 2 | 2 (100.0%) | 0 (0.0%) | 0 (0.0%) |
| | MS90/NTX.1 | 28 | 1 (3.6%) | | | 1 | 0 (0.0%) | 1 (100.0%) | 0 (0.0%) |
| RASH MACULAR | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 0 (0.0%) | | | 0 | 0 | 0 | 0 |
| | MS30/NTX.1 | 31 | 0 (0.0%) | | | 0 | 0 | 0 | 0 |
| | MS60/NTX.1 | 30 | 1 (3.3%) | | | 2 | 2 (100.0%) | 0 (0.0%) | 0 (0.0%) |
| | MS90/NTX.1 | 28 | 0 (0.0%) | | | 0 | 0 | 0 | 0 |
| SWEATING | PLACEBO | 31 | 0 (0.0%) | TRT | 0.286 | 0 | 0 | 0 | 0 |
| INCREASED | MS30 | 30 | 1 (3.3%) | | | 1 | 1 (100.0%) | 0 (0.0%) | 0 (0.0%) |
| | MS60 | 30 | 3 (10.0%) | | | 3 | 2 (66.7%) | 1 (33.3%) | 0 (0.0%) |
| | MS90 | 30 | 3 (10.0%) | | | 3 | 2 (66.7%) | 1 (33.3%) | 0 (0.0%) |
| | MS30/NTX.1 | 31 | 2 (6.5%) | | | 2 | 0 (0.0%) | 2 (100.0%) | 0 (0.0%) |
| | MS60/NTX.1 | 30 | 0 (0.0%) | | | 0 | 0 | 0 | 0 |
| | MS90/NTX.1 | 28 | 3 (10.7%) | | | 3 | 2 (66.7%) | 1 (33.3%) | 0 (0.0%) |

VASCULAR DISORDERS

| | | | | | | | | | |
|---|---|---|---|---|---|---|---|---|---|
| ALL EVENTS | PLACEBO | 31 | 0 (0.0%) | TRT | 0.199 | 0 | 0 | 0 | 0 |
| | MS30 | 30 | 1 (3.3%) | | | 1 | 0 (0.0%) | 1 (100.0%) | 0 (0.0%) |
| | MS60 | 30 | 1 (3.3%) | | | 1 | 1 (100.0%) | 0 (0.0%) | 0 (0.0%) |
| | MS90 | 30 | 3 (10.0%) | | | 3 | 1 (33.3%) | 2 (66.7%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 2 (7.1%) | | | 2 | 2 (100.0%) | 0 (0.0%) | 0 (0.0%) |
| FLUSHING | PLACEBO | 31 | 0 (0.0%) | TRT | 0.785 | 0 | 0 | 0 | 0 |
| | MS30 | 30 | 1 (3.3%) | | | 1 | 0 (0.0%) | 1 (100.0%) | 0 (0.0%) |
| | MS60 | 30 | 1 (3.3%) | | | 1 | 1 (100.0%) | 0 (0.0%) | 0 (0.0%) |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| HOT FLUSHES NOS | PLACEBO | 31 | 0 (0.0%) | TRT | 0.506 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 1 (100.0%) | 0 (0.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 1 (3.6%) | | | 1 | 1 (100.0%) | 0 (0.0%) | 0 (0.0%) |
| HYPOTENSION NOS | PLACEBO | 31 | 0 (0.0%) | TRT | 0.420 | 0 | 0 | 0 | 0 |
| | MS30 | 30 | 0 (0.0%) | | | 0 | 0 | 0 | 0 |
| | MS60 | 30 | 0 (0.0%) | | | 0 | 0 | 0 | 0 |
| | MS90 | 30 | 1 (3.3%) | | | 1 | 0 (0.0%) | 1 (100.0%) | 0 (0.0%) |
| | MS30/NTX.1 | 31 | 0 (0.0%) | | | 0 | 0 | 0 | 0 |
| | MS60/NTX.1 | 30 | 0 (0.0%) | | | 0 | 0 | 0 | 0 |
| | MS90/NTX.1 | 28 | 0 (0.0%) | | | 0 | 0 | 0 | 0 |

NOTE:
P-VALUE ARE FROM CHI-SQUARE TEST. P-VALUES FOR TREATMENT MAIN EFFECT AND SIGNIFICANT (P <= 0.05) PAIRWISE COMPARISONS ARE PRESENTED.

### EXAMPLE 8

In addition to the clinical studies described in Examples 1-7, several small pilot clinical studies were done with varying results.

One pilot study involved the co-administration of oral naltrexone and intrathecal morphine in patients with refractory chronic pain. This pilot study was performed to preliminarily evaluate and compare the analgesic effectiveness of intrathecal morphine and alone and in combination with two different doses of oral naltrexone in patients with chronic refractory pain. The 15 subject study had three treatment groups: a) morphine+placebo (5 patients); b) morphine+naltrexone 0.1 mg (3 patients); c) morphine+naltrexone 0.01 mg (7 patients). In this pilot study, all 15 patients had an indwelling intrathecal catheter and were currently receiving intrathecal morphine for refractory chronic pain. Each subject took one capsule of oral study medication every 12 hours for seven days. Subjects completed pain and side effect assessments before dosing and at 30 minutes, 1, 2, 3, 4, 5, 6, 7 and 8 hour after receiving the first dose of oral study medication. Subjects then completed assessments three times each day for the remaining six days of treatment, with a follow-up visit on the eighth day.

The efficacy and safety evaluations included: pain evaluation questionnaires (VAS), side effect scoring sheets, global efficacy evaluations (VAS), and adverse event assessments.

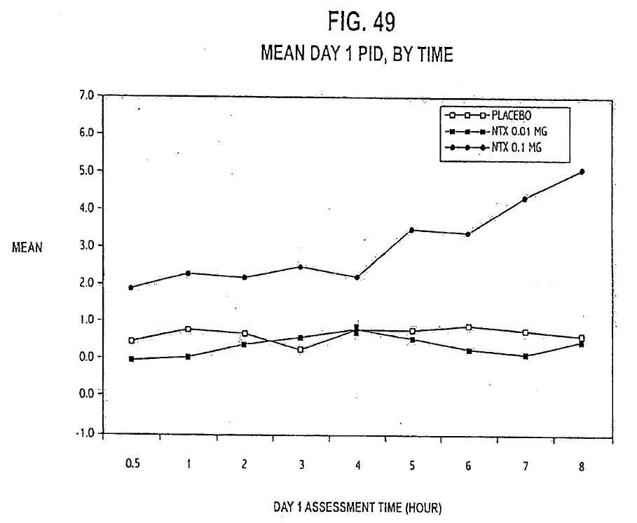

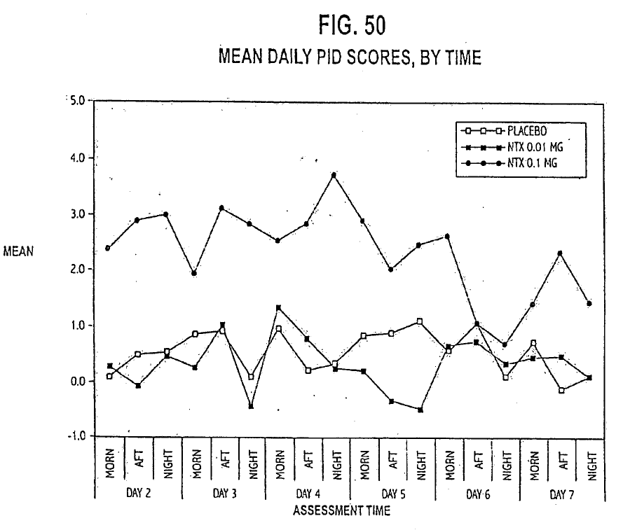

The mean pain intensity difference (PID) scores are shown by day and time in Tables 91 and 92, and FIGS. 49 and 50. Generally, the 0.1 mg NTX combination treatment group showed the highest mean PD scores.

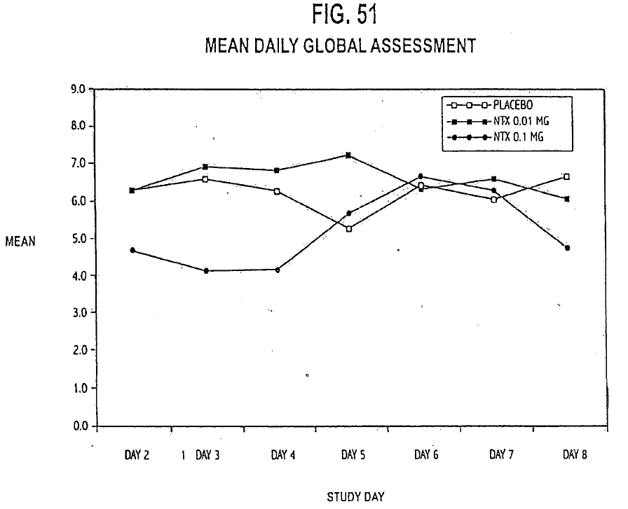

The mean daily global assessment of pain scores are shown for days 2-3 in Table 93 and FIG. 51. Particularly, for days 2-4, the 0.1 mg NTX combination treatment group showed the best (lowest mean) global assessment scores.

TABLE 91
Day 1 Mean Pain Intensity Difference (PID) Scores

| | Placebo | NTX 0.01 mg | NTX 0.1 mg |
|---|---|---|---|
| 0.5 | 0.44 | −0.04 | 1.87 |
| 1 | 0.76 | 0.03 | 2.27 |
| 2 | 0.64 | 0.34 | 2.17 |
| 3 | 0.22 | 0.56 | 2.47 |
| 4 | 0.76 | 0.71 | 2.23 |
| 5 | 0.74 | 0.49 | 3.47 |
| 6 | 0.86 | 0.24 | 3.37 |
| 7 | 0.70 | 0.10 | 4.30 |
| 8 | 0.64 | 0.39 | 5.03 |

TABLE 92
Day 1 Mean Pain Intensity Difference (PID) Scores

| | | Placebo | NTX 0.01 mg | NTX 0.1 mg |
|---|---|---|---|---|
| Day 2 | Morning | 0.10 | 0.27 | 2.37 |
| | Afternoon | 0.50 | −0.06 | 2.90 |
| | Night | 0.56 | 0.47 | 3.00 |
| Day 3 | Morning | 0.86 | 0.27 | 1.93 |
| | Afternoon | 0.96 | 1.06 | 3.13 |
| | Night | 0.10 | −0.44 | 2.83 |
| Day 4 | Morning | 0.96 | 1.33 | 2.53 |
| | Afternoon | 0.22 | 0.80 | 2.83 |
| | Night | 0.38 | 0.27 | 3.73 |
| Day 5 | Morning | 0.84 | 0.21 | 2.90 |
| | Afternoon | 0.88 | −0.33 | 2.03 |
| | Night | 1.08 | −0.50 | 2.47 |
| Day 6 | Morning | 0.56 | 0.66 | 2.60 |
| | Afternoon | 1.04 | 0.73 | 1.07 |
| | Night | 0.04 | 0.34 | 0.70 |
| Day 7 | Morning | 0.76 | 0.43 | 1.40 |
| | Afternoon | −0.14 | 0.47 | 2.30 |
| | Night | 0.12 | 0.10 | 1.43 |

Mean Daily Global Assessment Scores

| | Placebo | NTX 0.01 mg | NTX 0.1 mg |
|---|---|---|---|
| Day 2 | 6.32 | 6.27 | 4.70 |
| Day 3 | 6.58 | 6.93 | 4.13 |
| Day 4 | 6.26 | 6.81 | 4.17 |
| Day 5 | 5.24 | 7.23 | 5.67 |
| Day 6 | 6.48 | 6.30 | 6.63 |
| Day 7 | 6.06 | 6.56 | 6.23 |
| Day 8 | 6.62 | 6.06 | 4.73 |

In another pilot study, very low doses (e.g., 1 mg, 5 mg) of morphine in combination with naltrexone (0.01 mg or 0.001 mg) were administered for moderate to severe pain in patients following dental surgery. This pilot study was performed to investigate the analgesic efficacy (onset, peak, duration, and total effect) of 60 mg morphine alone, two different doses (0.01 mg or 0.001 mg) of naltrexone in combination with two different low doses (1 mg, 5 mg) of morphine, and placebo.

The 50 subject study was designed with six treatment groups: a) placebo (5 patients); b) morphine 60 mg (5 patients); c) morphine 1.0 mg and naltrexone 0.01 mg (10 patients); d) morphine 1.0 mg and naltrexone 0.001 mg (10 patients); e) morphine 5.0 mg and naltrexone 0.01 mg (10 patients); and f) morphine 5.0 mg and naltrexone 0.001 mg (10 patients). In this pilot study in the treatment of moderate to severe pain following extraction of 3 or 4 full or partial bony impacted third molars, a single oral dose of one of the treatments was administered when the patient was suffering moderate to severe postoperative pain. The observation period for efficacy was 8 hours post treatment and for safety was 24 hours post treatment.

The efficacy and safety evaluations included pain intensity, pain relief, global pain evaluation, evaluation of time to meaningful pain relief (stopwatch), visual analog scale (VAS), and adverse event assessment. This pilot study did not reveal any efficacy differences in the active treatment groups as compared with placebo.

In another pilot study of 25 subjects, the analgesic effects of morphine (5 mg, i.v.) in the presence of varying doses of an opioid antagonist (i.v. naloxone; 5 mg, 0.5 mg, 0.05 mg) as compared with morphine alone and placebo in healthy volunteers using the cold pain test.

Treatments were administered by 15 min i.v. infusion:

| | |
|---|---|
| Treatment A | 5 mg morphine sulphate + 4 × 0.9% saline solution |
| | (placebo |
| Treatment B | 5 mg morphine sulphate + 4 × 5 μg naloxone HCI |
| Treatment C | 5 mg morphine sulphate + 4 × 0.5 μg naloxone HCI |
| Treatment D | 5 mg morphine sulphate + 4 × 0.05 μg naloxone HCI |
| Treatment E | 0.9% saline solution (placebo) + 4 × 0.9% saline solution |

The cold pain test was performed pre-dose and at 20 minutes, 1 hr 20 in, 2 hr 20 in, 4 hr 20 min, and 6 hr 20 min post-dose on each of the five dosing occasions. In the test, a subject's hand is immersed in cold water usually over the range of 1 to 3° C. The initial sensation of cold is replace by a deep burning discomfort in the hand. It is thought that this is mediated by nociceptors in veins. The discomfort gradually builds to a plateau over 90 seconds or so and then either stays the same or decreases slightly.

The test statistic for each cold pain test was the cumulative area under the curve of the visual analogue scale-time profile from 0-120 seconds (AUC_{cpr}) calculated automatically by the cold pain test software. AUC_{cpr} values from the cold pain test were listed and plotted for each subject and treatment.

Minimum AUC_{cpt} and the time to achieve minimum AUC_{cpt} was determined for each subject and treatment dose level. This pilot study did not reveal any efficacy differences in the active treatment groups as compared with placebo.

### EXAMPLE 9

A study of tramadol alone and in combination with naltrexone is described in Example 10 of U.S. application Ser. No. 09/566,071, filed May 5, 2000 and 09/756,331, filed Jan. 8, 2001, as well as of PCT/US00/12493 [WO/00 67739] filed May 5, 2000, the entire disclosures of which are hereby incorporated by reference. A summary of exemplary study results follows.

In this study in human subjects with pain, tramadol hydrochloride (tramadol) was administered alone or in combination with various amounts (doses) of an opioid antagonist, naltrexone. In this study, one objective was to determine whether an opioid antagonist such as naltrexone hydrochloride (hereafter referred to in this example as naltrexone or NTX) enhanced the analgesic properties of tramadol hydrochloride (hereafter referred to in this example as tramadol or T) in human subjects/patients with pain following dental surgery. An additional objective was to evaluate whether an opioid antagonist such as NTX attenuated (e.g., reduced, blocked or prevented) tramadol's adverse side effects in humans.

Human subjects were randomized into one of the following five treatment groups:

Group 1: T (50 mg) with NTX (1 mg)

Group 2: T (50 mg) with NTX (0.1 mg)

Group 3: T (50 mg) with NTX (0.01 mg)

Group 4: T (50 mg) with Placebo

Group 5: Placebo with Placebo

All subjects with moderate to severe pain received one dose of study medication. Subjects received two capsules to take by mouth, one tramadol or placebo, the other naltrexone or placebo.

A pain assessment was performed pre-treatment. Following the dental surgery, the subject's pain level was assessed by a trained observer. The subject reported the initial pain intensity by both (1) verbalizing one pain category (0=none, 1=mild, 2=moderate or 3=severe), and (2) using a Visual Analog Scale (VAS) of 0-100 mm where 0=no pain and 100=worst pain imaginable, by placing a single slash on the scale. A pain assessment was also performed post-treatment.

The efficacy and safety evaluations included pain intensity, pain relief, global pain evaluation, evaluation of time to meaningful pain relief (stop watch), visual scale analog (VAS), and adverse event assessments. For the data analysis, certain pain parameters were computed as generally described above.

The placebo treatment group had the lowest mean 4-hour Total Pain Relief scores. All 4 of the active treatment groups exhibited mean 4-hour Total Pain Relief scores that were numerically higher than placebo. The combination treatments had a reverse dose-response relation in the mean 4-hour Total Pain Relief scores, i.e., the highest dose of NTX had the lowest mean 4-hour Total Pain Relief scores and the lowest dose of NTX had the highest mean 4-hour Total Pain Relief scores. The mean 4-hour Total Pain Relief scores for the 0.01-mg NTX and 0.1-mg NTX combination treatments were higher than that for the T alone treatment, whereas the 1.0-mg NTX combination treatment mean was lower than that for the T alone treatment.

The placebo treatment had the lowest mean 4-hour Sum of Pain Intensity Differences scores. All 4 of the active treatment groups exhibited improved profiles in mean 4-hour Sum of Pain Intensity Differences relative to placebo. The mean 4-hour Sum of Pain Intensity Differences scores for the 0.01-mg NTX and 0.1-mg NTX combination treatments were higher than that for the T alone treatment, whereas the 1.0-mg NTX combination treatment was lower than that for the T alone treatment. The patterns of the 6-hour and 8-hour Sum of Pain Intensity Differences scores were similar to those at 4 hours.

The 4, 6, and 8 hour Visual Analog Scale Sum of Pain Intensity Differences results were as follows. The placebo treatment had the lowest mean 4-hour VAS-Sum of Pain Intensity Differences. The 4 active treatment groups exhibited mean VAS-Sum of Pain Intensity Differences scores that were higher than that for the placebo. The mean 4-hour VAS-Sum of Pain Intensity Differences for the 3 NTX combination treatments was higher than that for T alone. The profiles of 6-hour and 8-hour VAS-Sum of Pain Intensity Differences scores were similar to those at 4 hours.

The placebo treatment had the lowest number of subjects who reached meaningful pain relief. In addition, all the combination treatment groups had higher numbers of subjects reaching meaningful pain relief than did the group that received T alone.

Whereas the hourly pain relief scores for the placebo treatment were generally flat, those for the active treatment groups were generally improving over time. There was separation between the placebo and the active treatment groups that continued throughout the 8-hour study period.

The majority of adverse events reported were categorized as gastrointestinal disorders (nausea or vomiting) or nervous system disorders (dizziness, headache or sedation).

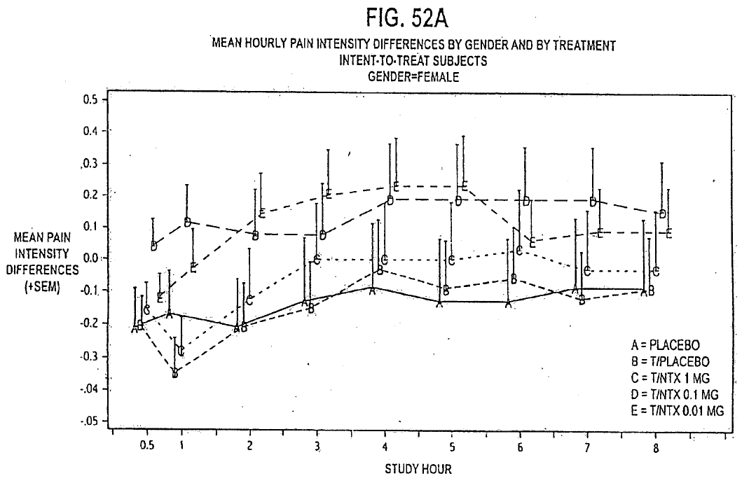

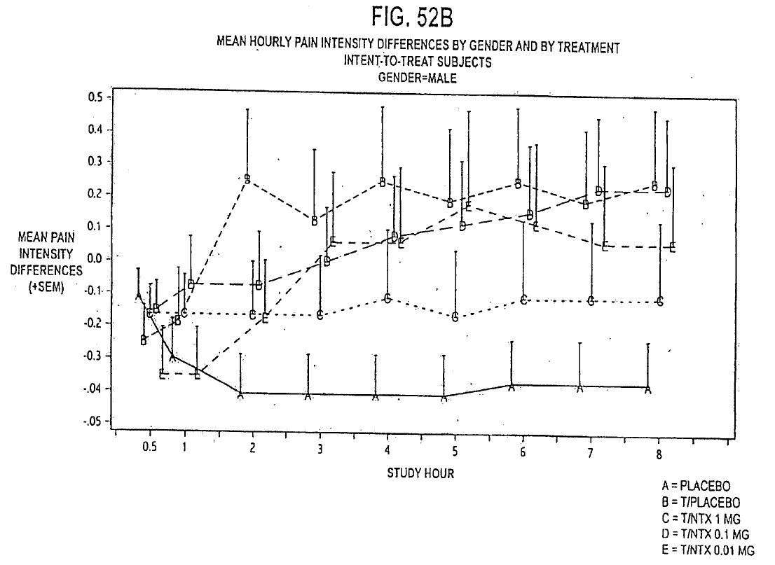

The results from this clinical study using tramadol alone and in combination with naltrexone were analyzed by gender. The results for females and males with respect to pain intensity difference (PID) scores are shown in Tables 93A and 93B and in FIGS. 52A and 52B.

TABLE 93A
Pain Intensity Difference (PID) Scores
Intent-to-Treat Population, Female Patients

| SUM OF PAIN INTENSITY DIFFERENCES | | | | | | | | | |
|---|---|---|---|---|---|---|---|---|---|
| | | | | | | | Overall | P-Value | P-Value |
| | | | | | | | P-Value | vs. | vs. |
| | N | Mean | SD | Median | Range | Source | [1] | Placebo | Tramadol |

SUM OF PAIN INTENSITY DIFFERENCES (0.5 HOURS)

| | | | | | | | | | |
|---|---|---|---|---|---|---|---|---|---|
| A) Placebo | 24 | −0.21 | 0.59 | 0.00 | −1-1 | TRT | 0.3257 | | |
| B) T (50 mg) with Placebo | 34 | −0.21 | 0.54 | 0.00 | −1-1 | A-B | | 0.9849 | |
| C) T (50 mg)/NTX 1.0 mg | 32 | −0.16 | 0.45 | 0.00 | −1-1 | B-C | | 0.6920 | 0.6789 |
| D) T (50 mg)/NTX 0.1 mg | 26 | 0.04 | 0.45 | 0.00 | −1-1 | B-D | | 0.0748 | 0.0555 |
| E) T (50 mg)/NTX 0.01 mg | 34 | −0.12 | 0.41 | 0.00 | −1-1 | B-E | | 0.4850 | 0.4552 |

SUM OF PAIN INTENSITY DIFFERENCES (1 HOUR)

| | | | | | | | | | |
|---|---|---|---|---|---|---|---|---|---|
| A) Placebo | 24 | −0.17 | 0.64 | 0.00 | −1-1 | TRT | 0.0372* | | |
| B) T (50 mg) with Placebo | 34 | −0.35 | 0.65 | 0.00 | −1-1 | A-B | | 0.2760 | |
| C) T (50 mg)/NTX 1.0 mg | 32 | −0.28 | 0.58 | 0.00 | −1-1 | B-C | | 0.5077 | 0.6494 |
| D) T (50 mg)/NTX 0.1 mg | 26 | 0.12 | 0.59 | 0.00 | −1-1 | B-D | | 0.1211 | 0.0056* |
| E) T (50 mg)/NTX 0.01 mg | 34 | −0.03 | 0.72 | 0.00 | −1-2 | B-E | | 0.4217 | 0.0386* |

SUM OF PAIN INTENSITY DIFFERENCES (2 HOURS)

| | | | | | | | | | |
|---|---|---|---|---|---|---|---|---|---|
| A) Placebo | 24 | −0.21 | 0.72 | 0.00 | −1-1 | TRT | 0.2525 | | |
| B) T (50 mg) with Placebo | 34 | −0.21 | 0.77 | 0.00 | −1-1 | A-B | | 0.9907 | |
| C) T (50 mg)/NTX 1.0 mg | 32 | −0.13 | 0.91 | 0.00 | −1-3 | B-C | | 0.6944 | 0.6759 |
| D) T (50 mg)/NTX 0.1 mg | 26 | 0.08 | 0.74 | 0.00 | −1-2 | B-D | | 0.2007 | 0.1683 |
| E) T (50 mg)/NTX 0.01 mg | 34 | 0.15 | 0.74 | 0.00 | −1-2 | B-E | | 0.0912 | 0.0655 |

SUM OF PAIN INTENSITY DIFFERENCES (3 HOURS)

| | | | | | | | | | |
|---|---|---|---|---|---|---|---|---|---|
| A) Placebo | 24 | −0.13 | 0.95 | 0.00 | −1-2 | TRT | 0.5012 | | |
| B) T (50 mg) with Placebo | 34 | −0.15 | 0.82 | 0.00 | −1-2 | A-B | | 0.9265 | |
| C) T (50 mg)/NTX 1.0 mg | 32 | 0.00 | 1.02 | 0.00 | −1-3 | B-C | | 0.6060 | 0.5060 |
| D) T (50 mg)/NTX 0.1 mg | 26 | 0.08 | 0.84 | 0.00 | −1-2 | B-D | | 0.4270 | 0.3387 |
| E) T (50 mg)/NTX 0.01 mg | 34 | 0.21 | 0.84 | 0.00 | −1-2 | B-E | | 0.1679 | 0.1064 |

SUM OF PAIN INTENSITY DIFFERENCES (4 HOURS)

| | | | | | | | | | |
|---|---|---|---|---|---|---|---|---|---|
| A) Placebo | 24 | −0.08 | 0.97 | 0.00 | −1-2 | TRT | 0.6085 | | |
| B) T (50 mg) with Placebo | 34 | −0.03 | 0.90 | 0.00 | −1-2 | A-B | | 0.8292 | |
| C) T (50 mg)/NTX 1.0 mg | 32 | 0.00 | 1.02 | 0.00 | −1-3 | B-C | | 0.7420 | 0.8986 |
| D) T (50 mg)/NTX 0.1 mg | 26 | 0.19 | 0.90 | 0.00 | −1-2 | B-D | | 0.2998 | 0.3646 |
| E) T (50 mg)/NTX 0.01 mg | 34 | 0.24 | 0.89 | 0.00 | −1-2 | B-E | | 0.2036 | 0.2454 |

SUM OF PAIN INTENSITY DIFFERENCES (5 HOURS)

| | | | | | | | | | |
|---|---|---|---|---|---|---|---|---|---|
| A) Placebo | 24 | −0.13 | 0.95 | 0.00 | −1-2 | TRT | 0.4673 | | |
| B) T (50 mg) with Placebo | 34 | −0.09 | 0.87 | 0.00 | −1-2 | A-B | | 0.8833 | |
| C) T (50 mg)/NTX 1.0 mg | 32 | 0.00 | 1.05 | 0.00 | −1-3 | B-C | | 0.6223 | 0.7030 |
| D) T (50 mg)/NTX 0.1 mg | 26 | 0.19 | 0.90 | 0.00 | −1-2 | B-D | | 0.2339 | 0.2527 |
| E) T (50 mg)/NTX 0.01 mg | 34 | 0.24 | 0.92 | 0.00 | −1-3 | B-E | | 0.1517 | 0.1570 |

SUM OF PAIN INTENSITY DIFFERENCES (6 HOURS)

| | | | | | | | | | |
|---|---|---|---|---|---|---|---|---|---|
| A) Placebo | 24 | −0.13 | 0.95 | 0.00 | −1-2 | TRT | 0.7751 | | |
| B) T (50 mg) with Placebo | 34 | −0.06 | 0.95 | 0.00 | −1-2 | A-B | | 0.7899 | |
| C) T (50 mg)/NTX 1.0 mg | 32 | −0.03 | 1.09 | 0.00 | −1-3 | B-C | | 0.5348 | 0.6947 |
| D) T (50 mg)/NTX 0.1 mg | 26 | 0.19 | 0.85 | 0.00 | −1-2 | B-D | | 0.2300 | 0.3017 |
| E) T (50 mg)/NTX 0.01 mg | 34 | 0.06 | 0.78 | 0.00 | −1-2 | B-E | | 0.4596 | 0.6027 |

SUM OF PAIN INTENSITY DIFFERENCES (7 HOURS)

| | | | | | | | | | |
|---|---|---|---|---|---|---|---|---|---|
| A) Placebo | 24 | −0.08 | 1.06 | 0.00 | −1-3 | TRT | 0.7077 | | |
| B) T (50 mg) with Placebo | 34 | −0.12 | 0.84 | 0.00 | −1-2 | A-B | | 0.8909 | |
| C) T (50 mg)/NTX 1.0 mg | 32 | −0.03 | 1.09 | 0.00 | −1-3 | B-C | | 0.8371 | 0.7085 |
| D) T (50 mg)/NTX 0.1 mg | 26 | 0.19 | 0.85 | 0.00 | −1-2 | B-D | | 0.3000 | 0.2059 |
| E) T (50 mg)/NTX 0.01 mg | 34 | 0.09 | 0.83 | 0.00 | −1-2 | B-E | | 0.4930 | 0.3661 |

SUM OF PAIN INTENSITY DIFFERENCES (8 HOURS)

| | | | | | | | | | |
|---|---|---|---|---|---|---|---|---|---|
| A) Placebo | 24 | −0.08 | 1.06 | 0.00 | −1-3 | TRT | 0.8312 | | |
| B) T (50 mg) with Placebo | 34 | −0.09 | 0.93 | 0.00 | −1-2 | A-B | | 0.9846 | |
| C) T (50 mg)/NTX 1.0 mg | 32 | −0.03 | 1.09 | 0.00 | −1-3 | B-C | | 0.8399 | 0.8085 |
| D) T (50 mg)/NTX 0.1 mg | 26 | 0.15 | 0.83 | 0.00 | −1-2 | B-D | | 0.3807 | 0.3311 |
| E) T (50 mg)/NTX 0.01 mg | 34 | 0.09 | 0.83 | 0.00 | −1-2 | B-E | | 0.5005 | 0.4464 |

PAIN INTENSITY SCORE: 0 = NONE, 1-MILD, 2 = MODERATE, 3 = SEVERE. THE PAIN INTENSITY DIFFERENCE (PID) AT EACH TIME POINT IS CALCULATED AS THE DIFFERENCE BETWEEN THE PAIN INTENSITY SCORE AT HOUR 0 AND THE SCORE AT OBSERVATION TIME.
[1] P-VALUES COMPARING ALL 5 TREATMENT GROUPS AND PAIRWISE COMPARISONS ARE DETERMINED USING ANOVA.
*SIGNIFICANCE IS AT 0.05 NOMINAL LEVEL.
LAST OBSERVATION CARRIED FORWARD METHOD IS USED TO IMPUTE MISSING VALUES.

TABLE 93B
Pain Intensity Difference (PID) Scores
Intent-to-Treat Population, Male Patients

| SUM OF PAIN INTENSITY DIFFERENCES | | | | | | | | P-Value | P-Value |
|---|---|---|---|---|---|---|---|---|---|
| | | | | | | | Overall | vs. | vs. |
| | N | Mean | SD | Median | Range | Source | P-Value | Placebo | Tramadol |

SUM OF PAIN INTENSITY DIFFERENCES (0.5 HOURS)

| | | | | | | | | | |
|---|---|---|---|---|---|---|---|---|---|
| A) Placebo | 27 | −0.11 | 0.42 | 0.00 | −1-1 | TRT | 0.5082 | | |
| B) T (50 mg) with Placebo | 16 | −0.25 | 0.45 | 0.00 | −1-0 | A-B | | 0.3464 | |
| C) T (50 mg)/NTX 1.0 mg | 18 | −0.17 | 0.38 | 0.00 | −1-0 | B-C | | 0.6956 | 0.6034 |
| D) T (50 mg)/NTX 0.1 mg | 26 | −0.15 | 0.46 | 0.00 | −1-1 | B-D | | 0.7389 | 0.5170 |
| E) T (50 mg)/NTX 0.01 mg | 17 | −0.35 | 0.61 | 0.00 | −1-1 | B-E | | 0.0964 | 0.5268 |

SUM OF PAIN INTENSITY DIFFERENCES (1 HOUR)

| | | | | | | | | | |
|---|---|---|---|---|---|---|---|---|---|
| A) Placebo | 27 | −0.30 | 0.61 | 0.00 | −1-1 | TRT | 0.6315 | | |
| B) T (50 mg) with Placebo | 16 | −0.19 | 0.66 | 0.00 | −1-1 | A-B | | 0.5901 | |
| C) T (50 mg)/NTX 1.0 mg | 18 | −0.17 | 0.51 | 0.00 | −1-1 | B-C | | 0.5059 | 0.9245 |
| D) T (50 mg)/NTX 0.1 mg | 26 | −0.08 | 0.74 | 0.00 | −1-1 | B-D | | 0.2137 | 0.5867 |
| E) T (50 mg)/NTX 0.01 mg | 17 | −0.35 | 0.61 | 0.00 | −1-1 | B-E | | 0.7749 | 0.4583 |

SUM OF PAIN INTENSITY DIFFERENCES (2 HOURS)

| | | | | | | | | | |
|---|---|---|---|---|---|---|---|---|---|
| A) Placebo | 27 | −0.41 | 0.64 | 0.00 | −1-1 | TRT | 0.1038 | | |
| B) T (50 mg) with Placebo | 16 | 0.25 | 0.86 | 0.00 | −1-2 | A-B | | 0.0068* | |
| C) T (50 mg)/NTX 1.0 mg | 18 | −0.17 | 0.71 | 0.00 | −1-1 | B-C | | 0.2968 | 0.1111 |
| D) T (50 mg)/NTX 0.1 mg | 26 | −0.08 | 0.84 | 0.00 | −1-1 | B-D | | 0.1140 | 0.1757 |
| E) T (50 mg)/NTX 0.01 mg | 17 | −0.18 | 0.73 | 0.00 | −1-1 | B-E | | 0.3252 | 0.1077 |

SUM OF PAIN INTENSITY DIFFERENCES (3 HOURS)

| | | | | | | | | | |
|---|---|---|---|---|---|---|---|---|---|
| A) Placebo | 27 | −0.41 | 0.64 | 0.00 | −1-1 | TRT | 0.1795 | | |
| B) T (50 mg) with Placebo | 16 | 0.13 | 0.89 | 0.00 | −1-2 | A-B | | 0.0379* | |
| C) T (50 mg)/NTX 1.0 mg | 18 | −0.17 | 0.79 | 0.00 | −1-1 | B-C | | 0.3264 | 0.2925 |
| D) T (50 mg)/NTX 0.1 mg | 26 | 0.00 | 0.85 | 0.00 | −1-1 | B-D | | 0.0675 | 0.6249 |
| E) T (50 mg)/NTX 0.01 mg | 17 | 0.06 | 0.90 | 0.00 | −1-2 | B-E | | 0.0634 | 0.8133 |

SUM OF PAIN INTENSITY DIFFERENCES (4 HOURS)

| | | | | | | | | | |
|---|---|---|---|---|---|---|---|---|---|
| A) Placebo | 27 | −0.41 | 0.64 | 0.00 | −1-1 | TRT | 0.1325 | | |
| B) T (50 mg) with Placebo | 16 | 0.25 | 0.93 | 0.00 | −1-2 | A-B | | 0.0194* | |
| C) T (50 mg)/NTX 1.0 mg | 18 | −0.11 | 0.90 | 0.00 | −1-2 | B-C | | 0.2694 | 0.2334 |
| D) T (50 mg)/NTX 0.1 mg | 26 | 0.08 | 0.98 | 0.00 | −1-2 | B-D | | 0.0471* | 0.5358 |
| E) T (50 mg)/NTX 0.01 mg | 17 | 0.06 | 0.97 | 0.00 | −1-2 | B-E | | 0.0890 | 0.5327 |

SUM OF PAIN INTENSITY DIFFERENCES (5 HOURS)

| | | | | | | | | | |
|---|---|---|---|---|---|---|---|---|---|
| A) Placebo | 27 | −0.41 | 0.64 | 0.00 | −1-1 | TRT | 0.1417 | | |
| B) T (50 mg) with Placebo | 16 | 0.19 | 0.91 | 0.00 | −1-2 | A-B | | 0.0465* | |
| C) T (50 mg)/NTX 1.0 mg | 18 | −0.17 | 0.86 | 0.00 | −1-2 | B-C | | 0.3996 | 0.2730 |
| D) T (50 mg)/NTX 0.1 mg | 26 | 0.12 | 1.03 | 0.00 | −1-2 | B-D | | 0.0446* | 0.8087 |
| E) T (50 mg)/NTX 0.01 mg | 17 | 0.18 | 1.24 | 0.00 | −1-3 | B-E | | 0.0465* | 0.9731 |

SUM OF PAIN INTENSITY DIFFERENCES (6 HOURS)

| | | | | | | | | | |
|---|---|---|---|---|---|---|---|---|---|
| A) Placebo | 27 | −0.37 | 0.69 | 0.00 | −1-1 | TRT | 0.1871 | | |
| B) T (50 mg) with Placebo | 16 | 0.25 | 0.93 | 0.00 | −1-2 | A-B | | 0.0420* | |
| C) T (50 mg)/NTX 1.0 mg | 18 | −0.11 | 1.02 | 0.00 | −1-3 | B-C | | 0.3743 | 0.2736 |
| D) T (50 mg)/NTX 0.1 mg | 26 | 0.15 | 1.08 | 0.00 | −1-2 | B-D | | 0.0484* | 0.7519 |
| E) T (50 mg)/NTX 0.01 mg | 17 | 0.12 | 1.05 | 0.00 | −1-2 | B-E | | 0.1019 | 0.6915 |

SUM OF PAIN INTENSITY DIFFERENCES (7 HOURS)

| | | | | | | | | | |
|---|---|---|---|---|---|---|---|---|---|
| A) Placebo | 27 | −0.37 | 0.69 | 0.00 | −1-1 | TRT | 0.1844 | | |
| B) T (50 mg) with Placebo | 16 | 0.19 | 0.91 | 0.00 | −1-2 | A-B | | 0.0697 | |
| C) T (50 mg)/NTX 1.0 mg | 18 | −0.11 | 1.02 | 0.00 | −1-3 | B-C | | 0.3791 | 0.3697 |
| D) T (50 mg)/NTX 0.1 mg | 26 | 0.23 | 1.14 | 0.00 | −1-2 | B-D | | 0.0255* | 0.8880 |
| E) T (50 mg)/NTX 0.01 mg | 17 | 0.06 | 1.03 | 0.00 | −1-2 | B-E | | 0.1537 | 0.7025 |

SUM OF PAIN INTENSITY DIFFERENCES (8 HOURS)

| | | | | | | | | | |
|---|---|---|---|---|---|---|---|---|---|
| A) Placebo | 27 | −0.37 | 0.69 | 0.00 | −1-1 | TRT | 0.1562 | | |
| B) T (50 mg) with Placebo | 16 | 0.25 | 0.93 | 0.00 | −1-2 | A-B | | 0.0447* | |
| C) T (50 mg)/NTX 1.0 mg | 18 | −0.11 | 1.02 | 0.00 | −1-3 | B-C | | 0.3805 | 0.2799 |
| D) T (50 mg)/NTX 0.1 mg | 26 | 0.23 | 1.14 | 0.00 | −1-2 | B-D | | 0.0259* | 0.9502 |
| E) T (50 mg)/NTX 0.01 mg | 17 | 0.06 | 1.03 | 0.00 | −1-2 | B-E | | 0.1550 | 0.5717 |

PAIN INTENSITY SCORE: 0 = NONE, 1-MILD, 2 = MODERATE, 3 = SEVERE. THE PAIN INTENSITY DIFFERENCE (PID) AT EACH TIME POINT IS CALCULATED AS THE DIFFERENCE BETWEEN THE PAIN INTENSITY SCORE AT HOUR 0 AND THE SCORE AT OBSERVATION TIME.
[1] P-VALUES COMPARING ALL 5 TREATMENT GROUPS AND PAIRWISE COMPARISONS ARE DETERMINED USING ANOVA.
*SIGNIFICANCE IS AT 0.05 NOMINAL LEVEL.
LAST OBSERVATION CARRIED FORWARD METHOD IS USED TO IMPUTE MISSING VALUES.

1. A method for enhancing the potency of an opioid agonist in a human subject comprising administering to the human subject an analgesic or subanalgesic amount of the agonist and an amount of an opioid antagonist effective to enhance the analgesic potency of the agonist without attenuating an adverse side effect of the agonist.

2. A method according to claim 1, wherein the opioid agonist is morphine, hydrocodone, oxycodone, or tamadol.

3. A method according to claim 1, wherein the opioid agonist is morphine.

4. A method according to claim 1, wherein the opioid antagonist is naltrexone, naloxone, or nalmefene.

5. A method according to claim 1, wherein the opioid antagonist is naltrexone.

6. A method according to claim 1, wherein the opioid antagonist is nalmefene.

7. A method according to claim 1, wherein the administration is oral, sublingual, intramuscular, subcutaneous, intravenous, transmucosal, or transdermal.

8. A method according to claim 1, wherein the administration is oral.

9. A method according to claim 1, wherein the human subject is male.

10. A method according to claim 1, wherein the human subject is female.

11. A method for attenuating an adverse side effect associated with administration of an opioid agonist to a human subject comprising administering to the human subject an analgesic or subanalgesic amount of the agonist and an amount of an opioid antagonist effective to attenuate the adverse side effect while maintaining analgesic potency of the agonist.

12. A method according to claim 11, wherein the adverse side effect is nausea, vomiting, dizziness, headache, sedation or pruritus.

13. A method according to claim 11, wherein the opioid agonist is morphine, hydrocodone, oxycodone or tramadol.

14. A method according to claim 11, wherein the opioid agonist is morphine.

15. A method according to claim 11, wherein the opioid antagonist naltrexone, naloxone, or nalmefene.

16. A method according to claim 11, wherein the opioid antagonist is naltrexone.

17. A method according to claim 11, wherein the opioid antagonist is nalmefene.

18. A method according to claim 11, wherein the administration is oral, sublingual, intramuscular, subcutaneous, intravenous, transmucosal or transdermal.

19. A method according to claim 11, wherein the administration is oral.

20. A method according to claim 11, wherein the analgesic potency of the agonist is maintained without increasing or decreasing the cumulative daily dose of the agonist relative to the antagonist.

21. A method according to claim 11, wherein the human subject is female.

22. A method according to claim 11, wherein the human subject is male.

23. A method for treating pain in a human subject comprising administering to the human subject an analgesic or subanalgesic amount of the agonist and an amount of an opioid antagonist effective to enhance the analgesic potency of the agonist without attenuating an adverse side effect of the agonist.

24. A method according to claim 23, wherein the opioid antagonist is morphine.

25. A method according to claim 23, wherein the opioid antagonist is naltrexone, naloxone, or nalmefene.

26. A method according to claim 23, wherein the opioid antagonist is naltrexone.

27. A method according to claim 23, wherein the opioid antagonist is nalmefene.

28. A method according to claim 23, wherein the administration is oral, sublingual, intramuscular, subcutaneous, intravenous, transmucosal or transdermal.

29. A method according to claim 23, wherein the administration is oral.

30. A method according to claim 23, wherein the human subject is male.

31. A method according to claim 23, wherein the human subject is female.

32. A method for treating pain with an opioid agonist and attenuating an adverse side effect of the agonist in a human subject comprising administering to the human subject an analgesic amount of the agonist and an amount of an opioid antagonist effective to attenuate the adverse side effect while maintaining analgesic potency of the agonist.

33. A method according to claim 32, wherein the opioid agonist is morphine, hydrocodone, oxycodone or tramadol.

34. A method according to claim 32, wherein the opioid agonist is morphine.

35. A method according to claim 32, wherein the opioid antagonist is naltrexone, naloxone, or nalmefene.

36. A method according to claim 32, wherein the opioid antagonist is naltrexone.

37. A method according to claim 32, wherein the opioid antagonist nalmefene.

38. A method according to claim 32, wherein the administration is oral, sublingual, intramuscular, subcutaneous, intravenous, transmucosal or transdermal.

39. A method according to claim 32, wherein the administration is oral.

40. A method according to claim 32, wherein the analgesic potency of the agonist is maintained without increasing or decreasing the cumulative daily doses of the agonist relative to the antagonist.

41. A method according to claim 32, wherein the human subject is female.

42. A method according to claim 32, wherein the human subject is male.

43-59. (canceled)

60. A method providing or enhancing pain relief in men comprising administering to a man a hypo-analgesic dose of a non-kappa opioid receptor agonist and a dose of an opioid antagonist that in combination provides or enhances pain relief.

61. A method according to claim 60, wherein the non-kappa opioid receptor agonist is a mu opioid receptor agonist.

62. A method according to claim 60, wherein the hypo-analgesic dose of the agonist is a non-analgesic dose or an anti-analgesic dose in men and an analgesic dose in women.

63. A method according to claim 60, wherein the dose of the antagonist prolongs the time to remedication.

64. A method according to claim 60, wherein the dose of the antagonist enhances the global evaluation of pain relief.

65. A method according to claim 60, wherein the agonist is morphine.

66. A method according to claim 60, wherein the agonist is naltrexone.

67. A method according to claim 60, wherein the pain relief is measured by the men using a categorical scale or a visual analog scale.

68-75. (canceled)

76. A method of enhancing pain relief in women comprising administering to a woman an analgesic dose of non-kappa opioid receptor agonist and a dose of opioid antagonist that in combination provides pain relief comparable to that of the agonist alone but with attenuation of one or more adverse side effects of the agonist.

77. A method according to claim 76, wherein the non-kappa opioid receptor agonist is a mu opioid receptor agonist.

78. A method according to claim 76, wherein the dose of the agonist is analgesic dose in women and a hypo-analgesic dose in men.

79. A method according to claim 76, wherein the dose of the antagonist prolongs the time to remedication.

80. A method according to claim 76, wherein the dose of the antagonist enhances the global evaluation of pain relief.

81. A method according to claim 76, wherein the agonist is morphine.

82. A method according to claim 76, wherein the antagonist is naltrexone.

83. A method according to claim 76, wherein the pain relief is measured by the women using a categorical scale or a visual analog scale.

84-102. (canceled)

103. A method for providing analgesia in a human subject administered a non-analgesic amount of an opioid agonist comprising concurrently administering with the agonist, an amount of opioid antagonist effective to provide analgesia.

104. A method according to claim 103, wherein the human subject is a man.

105. A method according to claim 104, wherein the opioid agonist is morphine.

106. A method according to claim 103, wherein the human subject is a woman.

107. A method according to claim 106, wherein the opioid agonist is tramadol.

108. A method of converting a hypo-analgesic dose of an opioid agonist into an analgesic dose of the agonist comprising administering to a human subject a combination of the hypo-analgesic dose of the agonist and an amount of an opioid antagonist sufficient to provide analgesia.

109. A method according to claim 108, wherein the opioid agonist is morphine, hydrocodone, oxycodone, or tramadol.

110. A method according to claim 108, wherein the opioid agonist is morphine.

111. A method according to claim 108, wherein the opioid antagonist is naltrexone, naloxone, or nalmefene.

112. A method according to claim 108, wherein the opioid antagonist is naltrexone.

113. A method according to claim 108, wherein the opioid antagonist is nalmefene.

114. A method according to claim 108, wherein the administration is oral, sublingual, intramuscular, subcutaneous, intravenous, transmucosal or transdermal.

115. A method according to claim 108, wherein the administration is oral.

116. A method according to claim 108, wherein the human subject is male.

117. A method according to claim 108, wherein the human subject is female.

118. A method according to claim 108, wherein the hypo-analgesic dose of the agonist is a non-analgesic dose or an anti-analgesic dose in men and an analgesic dose in women.

119. A method according to claim 108, wherein the dose of the antagonist prolongs the time to remedication.

120. A method according to claim 108, wherein the analgesia is measured by a pain relief score or a pain intensity difference score using a categorical scale or a visual analog scale.
